(19) United States
(12) Patent Application Publication
(10) Pub. No.: US 2025/0002541 A1
(43) Pub. Date: Jan. 2, 2025

(54) ENGINEERED CENTRAL NERVOUS SYSTEM COMPOSITIONS

(71) Applicants: The Broad Institute, Inc., Cambridge, MA (US); President and Fellows of Harvard College, Cambridge, MA (US); Massachusetts Institute of Technology, Cambridge, MA (US)

(72) Inventors: Pardis Sabeti, Cambridge, MA (US); Mohammadsharif Tabebordbar, Cambridge, MA (US); Simon Ye, Cambridge, MA (US); Alexandra Stanton, Cambridge, MA (US); Kim Lagerborg, Cambridge, MA (US)

(21) Appl. No.: 18/690,307

(22) PCT Filed: Sep. 8, 2022
(86) PCT No.: PCT/US22/76127

§ 371 Date: Mar. 8, 2024

Related U.S. Application Data
(60) Provisional application No. 63/242,014, filed on Sep. 8, 2021.
(60) Provisional application No. 63/322,191, filed on Mar. 21, 2022.

(51) Int. Cl. C07K 14/005; A61K 48/00; C12N 15/86

(52) U.S. Cl. CPC C07K 14/005; A61K 48/0041; C12N 15/86; C12N 2750/14122; C12N 2750/14143; C12N 2750/14145

(57) ABSTRACT

Described in several exemplary embodiments are compositions including a targeting moiety effective to target a central nervous system cell and formulations thereof. In certain embodiments, the targeting moiety is composed of a n-mer insert containing or being composed only of a P-motif. Also described in certain example embodiments are vector systems configured to generate polypeptides containing the one or more targeting moieties. Also described herein are methods of generating a targeting moiety effective to target a central nervous system cell and using the compositions containing the targeting moieties described herein, such as to deliver a cargo to a subject and/or treat a central nervous system disease, disorder, or system thereof.

### CROSS-REFERENCE TO RELATED APPLICATIONS

This application claims the benefit of and priority to U.S. Provisional Patent Application No. 63/242,014, filed on Sep. 8, 2021, and U.S. Provisional Patent Application No. 63/322,191, filed on Mar. 21, 2022, the contents of which are incorporated by reference herein in their entireties.

### REFERENCE TO AN ELECTRONIC SEQUENCE LISTING

This application contains a sequence listing filed in electronic form as an xml file entitled BROD-5465WP_ST26.xml, created on Sep. 8, 2022, and having a size of 10,872,431 bytes. The content of the sequence listing is incorporated herein in its entirety.

### TECHNICAL FIELD

The subject matter disclosed herein is generally directed to engineered central nervous system targeting compositions including, but not limited to, recombinant adeno-associated virus (AAV) vectors, and systems, compositions, and uses thereof.

### BACKGROUND

Recombinant AAVs (rAAVs) are the most commonly used delivery vehicles for gene therapy and gene editing. Nonetheless, rAAVs that contain natural capsid variants have limited cell tropism. Indeed, rAAVs used today mainly infect the liver after systemic delivery. Further, the transduction efficiency of conventional rAAVs in other cell-types, tissues, and organs by these conventional rAAVs with natural capsid variants is limited. Therefore, AAV-mediated polynucleotide delivery for diseased that affect cells, tissues, and organs other than the liver, such as the central nervous system) typically requires an injection of a large dose of virus (typically about 2×10¹⁴ vg/kg), which often results in liver toxicity. Furthermore, because large doses are required when using conventional rAAVs, manufacturing sufficient amounts of a therapeutic rAAV needed to dose adult patients is extremely challenging. Additionally, due to differences in gene expression and physiology, mouse and primate models respond differently to viral capsids. Transduction efficiency of different virus particles varies between different species, and as a result, preclinical studies in mice often do not accurately reflect results in primates, including humans. As such there exists a need for improved rAAVs for use in the treatment of various genetic diseases.

Citation or identification of any document in this application is not an admission that such a document is available as prior art to the present invention.

### SUMMARY

Described in certain example embodiments herein are compositions comprising a targeting moiety effective to target a central nervous system (CNS) cell, wherein the targeting moiety comprises an n-mer insert optionally comprising or consisting of a P-motif or a double valine motif, or both, wherein the P-motif comprises or consists of the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7, wherein the double valine motif comprises or consists of the amino acid sequence XₘX₁X₂VX₃X₄VX₅Xₙ, wherein X₁, X₂, X₃, Xₘ, and Xₙ, are each independently selected from any amino acid, wherein m is 0, 1, 2, or 3, and wherein n is 0, 1, 2, 3, 4, 5, 6, or 7; and optionally a cargo, wherein the cargo is coupled to or is otherwise associated with the targeting moiety.

In certain example embodiments, X₂ of the P motif is Q, P, E, or H. In certain example embodiments, X₁ of the P motif is a polar amino acid, optionally a polar uncharged amino acid. In certain example embodiments, X₃ of the P motif is a nonpolar amino acid. In certain example embodiments, X₁ of the double valine motif is R, K, V, or W. In certain example embodiments, X₂ of the double valine motif is T, S, V, Y or R.

In certain example embodiments, X₃ of the double valine motif is G, P, or S. In certain example embodiments, X₄ of the double valine motif is S, D, or T. In certain example embodiments, X₅ of the double valine motif is Y, G, S, or L.

In certain example embodiments, the targeting moiety comprises two or more n-mer inserts, optionally wherein each n-mer insert comprises or consists of a P-motif, wherein at least one of the P-motifs comprise or consists of the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7, optionally wherein X₂ of the P motif is Q, P, E, or H, optionally wherein the X₁ of the P motif is a polar amino acid, optionally a polar uncharged amino acid, and optionally wherein X₃ of the P motif is a nonpolar amino acid.

In certain example embodiments, the n-mer insert(s) and/or at least one P-motif and/or double valine motif is selected from any one n-mer insert and/or is encoded by a polynucleotide as set forth in one or more of SEQ ID NOs: 332-582 (Table 7), SEQ ID NOs: 583-8578 (Table 8), SEQ ID NOs: 3-819, 21-22, 24, 200, 202, 204, 212, 218, 224, 226, 228, 286, 234, 258, 260, 647, 649, 923, 1069, 1077, 1265, 2439, 2529, 2759, 3283, 3553, 3923, 4005, 4173, 4537, 4593, 4599, 4601, 4605, 4619, 4665, 4751, 4759, 4825, 4909, 4933, 5013, 5091, 5107, 5127, 5131, 5165, 5177, 5181, 5187, 5189, 5191, 5277, 5287, 5401, 5433, 5631, 5633, 5731, 5741, 5937, 6019, 6045, 6139, 6169, 6497, 7335, 8033, 8269, 8596-8613, (FIGS. 15A, 15B, 17A, 16A, 16B, 16C, and 19A-19C).

In certain example embodiments, the n-mer insert is 3-25 or 3-15 amino acids in length.

In certain example embodiments, X₁ of the P motif is S, T, N, Q, C, Y or A, X₂ of the P motif is Q, P, E, or H, X₃ is G, A, M, W, L, V, F, or I, or any combination thereof.

In certain example embodiments, the targeting moiety comprises a polypeptide, a polynucleotide, a lipid, a polymer, a sugar, or any combination thereof, wherein the polypeptide, the polynucleotide, the lipid, the polymer, the sugar, or any combination thereof is operably coupled to the n-mer insert(s).

In certain example embodiments, the targeting moiety comprises a viral polypeptide.

In certain example embodiments, the viral polypeptide is a capsid polypeptide.

In certain example embodiments, the n-mer insert(s) is/are incorporated into the viral polypeptide such that at least the n-mer insert is located between two amino acids of the viral polypeptide such that at least the n-mer insert is external to a viral capsid.

In certain example embodiments, the viral polypeptide is an adeno associated virus (AAV) polypeptide.

In certain example embodiments, the AAV polypeptide is an AAV capsid polypeptide.

In certain example embodiments, one or more of the n-mer insert(s) are each incorporated into the AAV polypeptide such that the n-mer insert, optionally the P motif(s) and/or double valine motif(s), is/are inserted between any two contiguous amino acids independently selected from amino acids 262-269, 327-332, 382-386, 452-460, 488-505, 527-539, 545-558, 581-593, 598-599, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, at least one n-mer insert is incorporated into the AAV polypeptide such that at least the P motif and/or double valine motif is inserted between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, the AAV capsid polypeptide is an engineered AAV capsid polypeptide having reduced or eliminated uptake in a non-CNS cell as compared to a corresponding wild-type AAV capsid polypeptide.

In certain example embodiments, the non-CNS cell is a liver cell or a dorsal root ganglion (DRG) neuron.

In certain example embodiments, the wild-type capsid polypeptide is an AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, the engineered AAV capsid polypeptide comprises one or more mutations that result in reduced or eliminated uptake in a non-CNS cell. In certain example embodiments, the one or more mutations are in position 267, in position 269, in position 272, in position 504, in position 505, in position 585, in position 590, or any combination thereof in the AAV9 capsid polypeptide (SEQ ID NO: 1) or in one or more positions corresponding thereto in a non-AAV9 capsid polypeptide.

In certain example embodiments, the non-AAV9 capsid polypeptide is an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, the mutation in position 267 in the AAV9 capsid polypeptide (SEQ ID NO: 1) or position corresponding thereto in a non-AAV9 capsid polypeptide is a G or X mutation to A, wherein X is any amino acid.

In certain example embodiments, the mutation in position 269 in the AAV9 capsid polypeptide (SEQ ID NO: 1) or position corresponding thereto in a non-AAV9 capsid polypeptide is an S or X to T mutation, wherein X is any amino acid.

In certain example embodiments, the mutation in position 272 in the AAV9 capsid polypeptide (SEQ ID NO: 1) or position corresponding thereto in a non-AAV9 capsid polypeptide is an N or to A mutation, wherein X is any amino acid.

In certain example embodiments, the mutation in position 504 in the AAV9 capsid polypeptide (SEQ ID NO: 1) or position corresponding thereto in a non-AAV9 capsid polypeptide is a G or X to A mutation, wherein X is any amino acid.

In certain example embodiments, the mutation in position 505 in the AAV9 capsid polypeptide (SEQ ID NO: 1) or position corresponding thereto in a non-AAV9 capsid polypeptide is a P or X to A mutation, wherein X is any amino acid.

In certain example embodiments, the mutation in position 585 in the AAV9 capsid polypeptide (SEQ ID NO: 1) or position corresponding thereto in a non-AAV9 capsid polypeptide is an R or X to Q mutation, wherein X is any amino acid.

In certain example embodiments, the mutation in position 590 in the AAV9 capsid polypeptide (SEQ ID NO: 1) or position corresponding thereto in a non-AAV9 capsid polypeptide is a Q or X to A mutation, wherein X is any amino acid.

In certain example embodiments, the engineered AAV capsid polypeptide is an engineered AAV9 capsid polypeptide comprising a mutation at position 267, position 269 or both of a wild-type AAV9 capsid polypeptide (SEQ ID NO: 1), wherein the mutation at position 267 is a G to A mutation and wherein the mutation at position 269 is an S to T mutation.

In certain example embodiments, the engineered AAV capsid polypeptide is an engineered AAV9 capsid polypeptide comprising a mutation at position 590 of a wild-type AAV9 capsid polypeptide (SEQ ID NO: 1), wherein the mutation at position 509 is a Q to A mutation.

In certain example embodiments, the engineered AAV capsid polypeptide is an engineered AAV9 capsid polypeptide comprising a mutation at position 504, position 505, or both of a wild-type AAV9 capsid protein (SEQ ID NO: 1), wherein the mutation at position 504 is a G to A mutation and wherein the mutation at position 505 is a P to A mutation.

In certain example embodiments, the composition is an engineered viral particle.

In certain example embodiments, the engineered viral particle is an engineered AAV viral particle. In certain example embodiments, the AAV viral particle is an engineered AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 viral particle.

In certain example embodiments, the optional cargo is capable of treating or preventing a CNS, an eye, or inner ear disease or disorder. In certain example embodiments, the optional cargo is also detargeted in a non-target cell, optionally a CNS cell.

In certain example embodiments, the optional cargo comprises one or more specific RNAi molecule binding sequences specific for an RNAi molecule endogenous to a non-target cell, wherein expression of the RNAi molecule(s) is/are enriched in the non-target cell as compared to a CNS cell and/or specific for synthetic RNAi molecule(s). In certain example embodiments, the RNAi molecule is not expressed in a CNS cell. In certain example embodiments, the non-target cell is a liver cell or a dorsal root ganglion neuron. In certain example embodiments, the RNAi molecule is miR183, miR-182, miR122, miR122a, miR99a, miR-26a, miR199a, miRNA-143, miR101a, miR-30c, or any combination thereof.

Described in certain example embodiments herein are vector systems comprising one or more polynucleotides, wherein at least one of the one or more polynucleotides encodes all or part of a targeting moiety effective to target a central nervous system (CNS) cell, wherein the targeting moiety comprises an n-mer insert optionally comprising or consisting of a P-motif or a double valine motif, or both, wherein the P-motif comprises or consists of the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7, wherein the double valine motif comprises or consists of the amino acid sequence XₘX₁X₂VX₃X₄VX₅Xₙ, wherein X₁, X₂, X₃, Xₘ, and Xₙ, are each independently selected from any amino acid, wherein m is 0, 1, 2, or 3, and wherein n is 0, 1, 2, 3, 4, 5, 6, or 7; and optionally, a regulatory element operatively coupled to one or more of the one or more polynucleotides.

In certain example embodiments, X₂ of the P motif is Q, P, E, or H. In certain example embodiments, X₁ of the P motif is a polar amino acid, optionally a polar uncharged amino acid. In certain example embodiments, X₃ of the P motif is a nonpolar amino acid.

In certain example embodiments, X₁ of the double valine motif is R, K, V, or W. In certain example embodiments, X₂ of the double valine motif is T, S, V, Y or R. In certain example embodiments, X₃ of the double valine motif is G, P, or S. In certain example embodiments, X₄ of the double valine motif is S, D, or T. In certain example embodiments, X₅ of the double valine motif is Y, G, S, or L.

In certain example embodiments, the targeting moiety comprises two or more n-mer inserts, optionally wherein each n-mer insert comprises or consists of a P-motif, wherein at least one of the P-motifs comprise or consists of the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7, optionally wherein X₂ of the P motif is Q, P, E, or H, optionally wherein the X₁ of the P motif is a polar amino acid, optionally a polar uncharged amino acid, and optionally wherein X₃ of the P motif is a nonpolar amino acid.

In certain example embodiments, the n-mer insert(s) and/or at least one P-motif and/or double valine motif is selected from any one n-mer insert and/or is encoded by a polynucleotide as set forth in one or more of SEQ ID NOs: 332-582 (Table 7), SEQ ID NOs: 583-8578 (Table 8), SEQ ID NOs: 3-819, 21-22, 24, 200, 202, 204, 212, 218, 224, 226, 228, 286, 234, 258, 260, 647, 649, 923, 1069, 1077, 1265, 2439, 2529, 2759, 3283, 3553, 3923, 4005, 4173, 4537, 4593, 4599, 4601, 4605, 4619, 4665, 4751, 4759, 4825, 4909, 4933, 5013, 5091, 5107, 5127, 5131, 5165, 5177, 5181, 5187, 5189, 5191, 5277, 5287, 5401, 5433, 5631, 5633, 5731, 5741, 5937, 6019, 6045, 6139, 6169, 6497, 7335, 8033, 8269, 8596-8613, (FIGS. 15A, 15B, 17A, 16A, 16B, 16C, and 19A-19C).

In certain example embodiments, the n-mer insert(s) are each 3-25 or 3-15 amino acids in length.

In certain example embodiments, X₁ of the P motif is S, T, N, Q, C, Y or A, X₂ of the P motif is Q, P, E, or H, X₃ is G, A, M, W, L, V, F, or I, or any combination thereof.

In certain example embodiments, the vector system further comprises a cargo.

In certain example embodiments, the cargo is a cargo polynucleotide and is optionally operatively coupled to one or more of the one or more polynucleotides encoding the targeting moiety.

In certain example embodiments, the vector system is a viral vector system and is capable of producing virus particles, virus particles that contain the cargo, or both.

In certain example embodiments, the vector system is capable of producing a polypeptide comprising one or more of the targeting moieties.

In certain example embodiments, the polypeptide is a viral polypeptide.

In certain example embodiments, the viral polypeptide is a capsid polypeptide.

In certain example embodiments, the capsid polypeptide is an adeno associated virus (AAV) capsid polypeptide. In certain example embodiments, the virus particles are AAV virus particles. In certain example embodiments, the AAV virus particles or AAV capsid polypeptide are engineered AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 viral particles or polypeptides.

In certain example embodiments, the n-mer insert(s) is/are incorporated into the viral polypeptide such that at least the n-mer insert is located between two amino acids of the viral polypeptide such that at least the n-mer insert is/are external to a viral capsid.

In certain example embodiments, the n-mer insert(s), optionally the P-motif(s) and/or double valine motif(s), are each inserted between any two contiguous amino acids independently selected from amino acids 262-269, 327-332, 382-386, 452-460, 488-505, 527-539, 545-558, 581-593, 598-599, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, the at least one polynucleotide that encodes all or part of a targeting moiety is inserted between the codons corresponding to amino acid 588 and 589 in the AAV9 capsid polynucleotide or in an analogous position in an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, the AAV capsid polypeptide is an engineered AAV capsid polypeptide having reduced or eliminated uptake in a non-CNS cell as compared to a corresponding wild-type AAV capsid polypeptide. In certain example embodiments, the non-CNS cell is a liver cell or a dorsal root ganglion (DRG) neuron. In certain example embodiments, the wild-type capsid polypeptide is an AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, the engineered AAV capsid polypeptide comprises one or more mutations that result in reduced or eliminated uptake in a non-CNS cell. In certain example embodiments, the one or more mutations are in position 267, in position 269,in position 272,in position 504, in position 505, in position 585, in position 590, or any combination thereof in the AAV9 capsid polypeptide (SEQ ID NO: 1) or in one or more positions corresponding thereto in a non-AAV9 capsid polypeptide. In certain example embodiments, the non-AAV9 capsid polypeptide is an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, the mutation in position 267 in the AAV9 capsid polypeptide (SEQ ID NO: 1) or position corresponding thereto in a non-AAV9 capsid polypeptide is a G or X mutation to A, wherein X is any amino acid.

In certain example embodiments, the mutation in position 269 in the AAV9 capsid polypeptide (SEQ ID NO: 1) or position corresponding thereto in a non-AAV9 capsid polypeptide is an S or X to T mutation, wherein X is any amino acid.

In certain example embodiments, the mutation in position 272 in the AAV9 capsid polypeptide (SEQ ID NO: 1) or position corresponding thereto in a non-AAV9 capsid polypeptide is an N or to A mutation, wherein X is any amino acid.

In certain example embodiments, the mutation in position 504 in the AAV9 capsid polypeptide (SEQ ID NO: 1) or position corresponding thereto in a non-AAV9 capsid polypeptide is a G or X to A mutation, wherein X is any amino acid.

In certain example embodiments, the mutation in position 505 in the AAV9 capsid polypeptide (SEQ ID NO: 1) or position corresponding thereto in a non-AAV9 capsid polypeptide is a P or X to A mutation, wherein X is any amino acid.

In certain example embodiments, the mutation in position 585 in the AAV9 capsid polypeptide (SEQ ID NO: 1) or position corresponding thereto in a non-AAV9 capsid polypeptide is an R or X to Q mutation, wherein X is any amino acid.

In certain example embodiments, the mutation in position 590 in the AAV9 capsid polypeptide (SEQ ID NO: 1) or position corresponding thereto in a non-AAV9 capsid polypeptide is a Q or X to A mutation, wherein X is any amino acid.

In certain example embodiments, the engineered AAV capsid polypeptide is an engineered AAV9 capsid polypeptide comprising a mutation at position 267, position 269, or both of a wild-type AAV9 capsid polypeptide (SEQ ID NO: 1), wherein the mutation at position 267 is a G to A mutation and wherein the mutation at position 269 is an S to T mutation.

In certain example embodiments, the engineered AAV capsid polypeptide is an engineered AAV9 capsid polypeptide comprising a mutation at position 590 of a wild-type AAV9 capsid polypeptide (SEQ ID NO: 1), wherein the mutation at position 509 is a Q to A mutation.

In certain example embodiments, engineered AAV capsid protein is an engineered AAV9 capsid polypeptide comprising a mutation at position 504, position 505, or both of a wild-type AAV9 capsid polypeptide (SEQ ID NO: 1), wherein the mutation at position 504 is a G to A mutation and wherein the mutation at position 505 is a P to A mutation.

In certain example embodiments, the cargo comprises one or more specific RNAi molecule binding sequences specific for an RNAi molecule endogenous to a non-target cell, wherein expression of the RNAi molecule(s) is/are enriched in the non-target cell as compared to a CNS cell and/or specific for synthetic RNAi molecule(s). In certain example embodiments, the RNAi molecule is not expressed in a CNS cell. In certain example embodiments, the non-target cell is a liver cell or a dorsal root ganglion neuron. In certain example embodiments, the RNAi molecule is miR183, miR-182, miR122, miR122a, miR99a, miR-26a, miR199a, miRNA-143, miR101a, miR-30c, or any combination thereof.

In some embodiments, the viral polypeptide is optionally a capsid polypeptide, wherein the composition is modified to include one or more azides, have a reduced number of one or more oxidation susceptible residues, wherein the oxidation susceptible residues are optionally Met, Tyr, Trp, His, Cys or any combination thereof; is PEGylated, or is otherwise functionalized for PEGylation; comprises one or more oligonucleotides tethered via click chemistry to the composition, optionally viral polypeptide; or any combination thereof.

In certain example embodiments, the viral vector and/or cargo is engineered to include one or more cis-acting elements or modifications, optionally a reduced number of CpG islands; one or more TLR9i oligonucleotides, optionally in one or both of the inverted terminal repeats of the vector system; one or more regulatory elements to modify cargo expression; a reduced number of ITR mimicking harpin or other structures; or any combination thereof.

In certain example embodiments, the vector comprising the one or more polynucleotides does not comprise splice regulatory elements.

In certain example embodiments, the vector system further comprises a polynucleotide that encodes a viral rep protein. In certain example embodiments, the viral rep polypeptide is an AAV rep protein. In certain example embodiments, the polynucleotide that encodes the viral rep polypeptide is on the same vector or a different vector as the one or more polynucleotides encoding the targeting moiety or portion thereof. In certain example embodiments, the polynucleotide that encodes the viral rep protein is operatively coupled to a regulatory element.

In certain example embodiments, the vector system is capable of producing a composition or portion thereof as described in any one of the preceding paragraphs or elsewhere herein.

Described in certain example embodiments herein are polynucleotides that encode a composition or portion thereof as described in any one of the preceding paragraphs or elsewhere herein.

Described in certain example embodiments herein are polypeptides encoded by, produced by, or both by a vector system as described in any one of the preceding paragraphs or elsewhere herein or a polynucleotide as described in any one of the preceding paragraphs or elsewhere herein.

In certain example embodiments, the polypeptide is a viral polypeptide. In certain example embodiments, the viral polypeptide is an AAV polypeptide. In certain example embodiments, the polypeptide is coupled to or otherwise associated with a cargo.

In certain example embodiments, the cargo comprises one or more specific RNAi molecule binding sequences specific for an RNAi molecule endogenous to a non-target cell, wherein expression of the RNAi molecule(s) is/are enriched in the non-target cell as compared to a CNS cell and/or specific for synthetic RNAi molecule(s). In certain example embodiments, the RNAi molecule is not expressed in a CNS cell. In certain example embodiments, the non-target cell is a liver cell or a dorsal root ganglion neuron. In certain example embodiments, the RNAi molecule is miR183, miR-182, miR122, miR122a, miR99a, miR-26a, miR199a, miRNA-143, miR101a, miR-30c, or any combination thereof.

In certain example embodiments, the polypeptide includes one or more azides; has a reduced number of one or more oxidation susceptible residues, wherein the oxidation susceptible residues are optionally Met, Tyr, Trp, His, Cys or any combination thereof; is PEGylated, or is otherwise functionalized for PEGylation; comprises one or more oligonucleotides tethered via click chemistry to the composition, optionally viral polypeptide; or any combination thereof.

Described in certain example embodiments herein are particles produced by a vector system as described in any one of the preceding paragraphs or elsewhere herein, optionally including a polypeptide s described in any one of the preceding paragraphs or elsewhere herein. In certain example embodiments, the particle is a viral particle. In certain example embodiments, the viral particle is an adeno-associated virus (AAV) particle, lentiviral particle, or a retroviral particle. In certain example embodiments, the particle comprises a cargo. In certain example embodiments, the viral particle has a central nervous system (CNS) tropism.

In certain example embodiments, the cargo comprises one or more specific RNAi molecule binding sequences specific for an RNAi molecule endogenous to a non-target cell, wherein expression of the RNAi molecule(s) is/are enriched in the non-target cell as compared to a CNS cell and/or specific for synthetic RNAi molecule(s). In certain example embodiments, the RNAi molecule is not expressed in a CNS cell. In certain example embodiments, non-target cell is a liver cell or a dorsal root ganglion neuron. In certain example embodiments, the RNAi molecule is miR183, miR-182, miR122, miR122a, miR99a, miR-26a, miR199a, miRNA-143, miR101a, miR-30c, or any combination thereof.

In certain example embodiments, the polypeptide includes one or more azides; has a reduced number of one or more oxidation susceptible residues, wherein the oxidation susceptible residues are optionally Met, Tyr, Trp, His, Cys or any combination thereof; is PEGylated, or is otherwise functionalized for PEGylation; comprises one or more oligonucleotides tethered via click chemistry to the composition, optionally viral polypeptide; or any combination thereof.

In certain example embodiments of the vector system, polynucleotide, polypeptide or any combination thereof, the cargo is capable of treating or preventing a CNS, an eye, or an inner ear disease or disorder. In certain example embodiments, the cargo is also detargeted in a non-target cell, optionally a CNS cell.

Described in certain example embodiments herein are cell(s) comprising a composition as described in any one of the preceding paragraphs or elsewhere herein; a vector system as described in any one of the preceding paragraphs or elsewhere herein; a polynucleotide as described in any one of the preceding paragraphs or elsewhere herein; a polypeptide as described in any one of the preceding paragraphs or elsewhere herein; a particle as described in any one of the preceding paragraphs or elsewhere herein; or any combination thereof. In certain example embodiments, the cell(s) is/are prokaryotic. In certain example embodiments, the cell(s) is/are eukaryotic.

Described in certain example embodiments herein are pharmaceutical formulation(s) comprising a composition as described in any one of the preceding paragraphs or elsewhere herein; a vector system as described in any one of the preceding paragraphs or elsewhere herein; a polynucleotide as described in any one of the preceding paragraphs or elsewhere herein; a polypeptide as described in any one of the preceding paragraphs or elsewhere herein; a particle as described in any one of the preceding paragraphs or elsewhere herein; a cell as described in any one of the preceding paragraphs or elsewhere herein; or any combination thereof; and a pharmaceutically acceptable carrier.

Described in certain example embodiments herein are methods of treating or preventing a central nervous system, an eye, or an inner ear disease, disorder, or a symptom thereof comprising administering, to the subject in need thereof, a composition as described in any one of the preceding paragraphs or elsewhere herein; a vector system as described in any one of the preceding paragraphs or elsewhere herein; a polynucleotide as described in any one of the preceding paragraphs or elsewhere herein; a polypeptide as described in any one of the preceding paragraphs or elsewhere herein; a particle as described in any one of the preceding paragraphs or elsewhere herein; a cell as described in any one of the preceding paragraphs or elsewhere herein; a pharmaceutical formulation as described in any one of the preceding paragraphs or elsewhere herein; or any combination thereof.

In certain example embodiments, the central nervous system disease or disorder comprises a secondary muscle disease, disorder, or symptom thereof.

In certain example embodiments, the central nervous system disease or disorder is Friedreich's Ataxia, Dravet Syndrome, Spinocerebellar Ataxia Type 3, Niemann Pick Type C, Huntington's Disease, Pompe Disease, Myotonic Dystrophy Type 1, Glut1 Deficiency Syndrome (De Vivo Syndrome), Tay-Sachs, Spinal Muscular Atrophy, Alzheimer's disease, Amyotrophic lateral sclerosis (ALS), Danon disease, Rett Syndrome, Angleman Syndrome, infantile neuronal dystorpy, Gaucher's disease, Krabbe disease, metachromatic leukodystrophy, Salla disease, Farber disease or Spinal Musular Atrophy with progressive myoclonic Epilepsy (also reffered to as Jankovic-Rivera syndrome, Unverricht-Lundborg disease, AADC deficiency, Parkinson's disease, Batten disease, a neuronal ceroid lipofuscinosis disease, giant axonal neuropathy, a mucopolysaccharidosis disease (e.g., Hurler syndrome, MPS III A-D), neurofibromatosis, a spinocerebellar ataxia disease, Sandoff disease, GM2 gangliosidosis, Canavan disease, Cockayne syndrome, or any combination thereof

In certain example embodiments, the eye disease or disorder is Stargardt disease, a Leber's congenital amaurosis (LCA) (e.g., Leber's congenital amaurosis type 2, LEBER CONGENITALAMAUROSIS (LCA) ANDEARLY-ONSET SEVERE RETINALDYSTROPHY (EOSRD)), Choroideremia, a macular degeneration, diabetic retinopathy, a retinopathy, vitelliform macular dystrophy, a macular dystrophy, Sorsby's fundus dystrophy, cataracts, glaucoma, optic neuropathies, Marfan syndrome, myopia, polypoidal choroidal vasculopathies, retinitis pigmentosa, uveal melanoma, X-linked retinoschisis, pattern dystrophy, achromatopsia, Blue cone monochromatism, Bornholm eye disease, ADGUCA1A-associated COD/CORD, autosomal dominant PRPH2 associated CORD, X-linkedRPGR-associatedCOD/CORD, fundus albipunctatus, Enhanced S-conesyndrome, Bietti crystalline comeoretinaldystorphy, or any combination thereof.

In certain example embodiments, the inner ear disease or disorder is GJB-2 deafness, Jeryell and Lange-Nielsen syndrome, Usher syndrome, Alport syndrome, Branchio-oto-renal syndrome, Waardenburg syndrome, Pendred syndrome, Stickler syndrome, Treacher Collins syndrome, CHARGE syndrome, Norrie disease, Perrault syndrome, Autosomal dominant Nonsyndromic hearing loss, utosomal Recessive Nonsyndromic Hearing Loss, X-linked nonsyndromic hearing loss, an auditory neuropathy, a congenital hearing loss, or any combination thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows the adeno-associated virus (AAV) transduction mechanism, which results in production of mRNA from the transgene.

FIG. 2 shows a graph that can demonstrate that mRNA-based selection of AAV variants can be more stringent than DNA-based selection. The virus library was expressed under the control of a CMV promoter.

FIGS. 3A-3B show graphs that can demonstrate a correlation between the virus library and vector genome DNA (FIG. 3A) and mRNA (FIG. 3B) in the liver.

FIGS. 4A-4F show graphs that can demonstrate capsid variants present at the DNA level, and expressed at the mRNA level identified in different tissues. For this experiment, the virus library was expressed under the control of a CMV promoter.

FIGS. 5A-5C show graphs that can demonstrate capsid mRNA expression in different tissues under the control of cell-type specific promoters (as noted on x-axis). CMV was included as an exemplary constitutive promoter. CK8 is a muscle-specific promoter. MHCK7 is a muscle-specific promoter. hSyn is a neuron specific promoter. Expression levels from the cell type-specific promoters have been normalized based on expression levels from the constitutive CMV promoter in each tissue.

FIGS. 6A-6B show (FIG. 6A) a schematic demonstrating embodiments of a method of producing and selecting capsid variants for tissue-specific gene delivery across species and (FIG. 6B) a schematic demonstrating benchmarking of the top selected capsids.

FIG. 7 shows a schematic demonstrating embodiments of generating an AAV capsid variant library, particularly insertion of a random n-mer (n=3-15 amino acids) into a wild-type AAV, e.g., AAV9.

FIG. 8 shows a schematic demonstrating embodiments of generating an AAV capsid variant library, particularly variant AAV particle production. Each capsid variant encapsulates its own coding sequence as the vector genome.

FIG. 9 shows schematic vector maps of representative AAV capsid plasmid library vectors (see e.g., FIG. 8) that can be used in an AAV vector system to generate an AAV capsid variant library.

FIG. 10 shows a graph that can demonstrate the viral titer (calculated as AAV9 vector genome/15 cm dish) produced by constructs containing different constitutive and cell-type specific mammalian promoters.

FIGS. 11A-11P show results from benchmarking the top selected capsids from the first and second round of selection.

FIGS. 12A-12C show a comparison of transduction between the EVGPTQGTVR (SEQ ID NO: 332) capsid insert variant with AAV9 in NHP tissues.

FIGS. 13A-13C show a comparison of the vector genome biodistribution between the EVGPTQGTVR (SEQ ID NO: 332) capsid insert variant with AAV9 in NHP tissues.

FIG. 14A-14B—The DELIVER strategy selects for AAV capsid variants with an enhanced ability to transcribe transgene mRNA in the tissue of interest. (FIG. 14A) (SEQ ID NO: 8614) Map of self-packaging capsid library construct for DELIVER. (FIG. 14B) Schematic of selection using DELIVER.

FIG. 15A-15F—Selection with DELIVER yields potent CNS-tropic capsid variants in multiple mouse strains. (FIGS. 15A and 15B) Amino acid sequence and logo of the 7-mer insert in the 10 most enriched capsid variants with the (FIG. 15A) (SEQ ID NO: 3-8) AQ or (FIG. 15B) (SEQ ID NO: 19, 21-22, 24, 647, 649) DG prefix in the brain of 8 week old C57BL6J and BALB/cJ mice injected with 1E+12 vectorgenomes (vg) virus library following two rounds of selection with DELIVER. Sequences with the same color in each table are encoded by synonymous DNA codons. (FIG. 15C) Predicted structure of the VR-VIII surface loops of AAV9, MDV1A, and MDV1B. (FIG. 15D) Fold difference in eGFP mRNA expression from MDV1A compared to AAV9 in the brain and spinal cord of male and female 8 week old C57BL/6J and BALB/cJ mice injected with 1E+12 vg of MDV1A- or AAV9-CMV-eGFP. Dashed red line represents AAV9-CMV-eGFP expression normalized to 1. Data are represented as mean±SD (n=3-4); *p<0.05, **p<0.01 (Welch's t-test between MDV1A- and AAV9-injected mice with Holm-Šidák MCT). (FIG. 15E) Quantification of transgene delivery efficiency, expressed as vector genomes per diploid genome, of MDV1A- and AAV9-CMV-eGFP in the brain and spinal cord of 8 week old C57BL/6J and BALB/cJ mice injected with 1E+12 vg of MDV1A- or AAV9-CMV-eGFP. Data are represented as mean±SD (n=3-4); *p<0.05, **p<0.01 (Welch's t-test between MDV1A- and AAV9-injected mice with Holm-Šidák MCT). (FIG. 15F) Representative images of mouse brain sagittal sections immunostained for eGFP, from 8 week old C57BL/6J and BALB/cJ mice injected with 5E+11 vg of MDV1A- or AAV9-CMV-eGFP. Blue insets show magnified features in the cortex. Scale bar: 1 mm.

FIG. 16A-16D—The Proline Arginine Loop (PAL) family of neurotropic capsid variants in cynomolgus macaques emerges after selection with DELIVER (see also FIG. 19A-19C). (FIG. 16A) (SEQ ID NO: 200, 202, 204, 212, 218, 224, 228, 234) DNA sequence and corresponding peptide sequence logo of the 7-mer insert in the 10 most enriched DNA sequences of capsid variants in the central nervous system of cynomolgus macaques injected with 3E+13 vg/kg virus library following two rounds of selection with DELIVER. Sequences with the same color are encoded by synonymous DNA codons. (FIG. 16B) (SEQ ID NO: 200, 204, 286, 4005, 4357, 4593, 4599, 4601) Amino acid sequence and logo of the 7-mer insert in the 10 most enriched amino acid-level capsid variants in the macaque CNS. The rank of each variant corresponds to the sum of the ranks of two synonymous DNA sequences. (FIG. 16C—SEQ ID NO: 200, 204, 226, 234, 258, 260, 923, 1265, 2759, 3923, 4593, 4599, 4713, 5277, 5433, 5741, 5937, 6019)

FIG. 17A-17E. Macaque-derived variants outperform AAV9 and mouse- and marmoset-derived variants in transduction of the macaque but not the mouse central nervous system (see also FIG. 20A-20B). (FIG. 17A)(SEQ ID NO: 8596-8613) Pool of capsid variants injected for characterization of the top mouse- and macaque-derived neurotropic variants. (FIG. 17B) Schematic of the barcoded human frataxin transgene and strategy for assessing the performance of top variants in cynomolgus macaques and C57BL/6J and BALB/cJ mice. (FIG. 17C-17E) Fold difference in within-individual hFXN mRNA expression from different variants normalized to AAV9 in various tissues of (FIG. 17C) C57BL/6J mice, (D) BALB/cJ mice, and (E) cynomolgus macaques. Dashed red line represents AAV9-CBh-hFXN expression normalized to 1. Data are represented as mean±SD (n=3 macaques, n=4-7 mice); *p<0.05, **p<0.01 (one-way ANOVA with Dunnett's MCT and AAV9 as the control).

FIG. 18A-18H—Second-generation capsid variant PAL2 transduces the central nervous system of one macaque in a head-to-head experiment with AAV9. (FIG. 18A) Heatmap of PAL2 transgene mRNA expression and vector genome abundance normalized to AAV9. Data are log₂-transformed. (FIG. 18B) Immunostaining a coronal section of macaque brain hemisphere for the hFXN-HA transgene delivered by PAL2 suggests widespread and uniform transduction. Scale bar: 1 cm. (FIG. 18C-18E) Localization of hFXN-HA expression with respect to NeuN+ neurons in the macaque (FIG. 18C) parietal cortex, (FIG. 18D) hippocampus, and (FIG. 18E) spinal cord. Scale bars: 100 pm. (FIG. 18F) Localization of hFXN-HA expression with respect to rhodopsin+ photoreceptors in the macaque retina. Scale bars: 100 μm. (FIG. 18G) Representative spinal cord sections with pathology WNL (within normal limits). Scale bar: 200 μm. (FIG. 18H) Representative DRG sections with pathology WNL (within normal limits). Scale bar: 100 μm.

FIG. 19A-19C—Selection for capsid variants with neurotropic properties in cynomolgus macaques yields diverse families of motifs. (FIGS. 19A and 19B) Amino acid sequence and logo of the 7-mer insert in the 10 most enriched capsid variants in the (FIG. 19A) (SEQ ID NO: 260, 1069, 4665, 4751, 4909, 5013, 5107, 5191, 5287, 5401) cerebellum and (FIG. 19B) (SEQ ID NO: 224, 4759, 4971, 5091, 5127, 5165, 5177, 5181, 5187, 5189) spinal cord of cynomolgus macaques injected with 3E+13 vg/kg virus library following two rounds of selection with DELIVER. The rank of each variant corresponds to the sum of the ranks of two synonymous DNA sequences. (FIG. 19C) (SEQ ID NO: 971, 1077, 2439, 2529, 3103, 3283, 3553, 4605, 4619, 4629, 4825, 4933, 5131, 5209, 5233, 5341, 5367, 5461, 5547, 5631, 5633, 5731, 5959, 6001, 6045, 6139, 6169, 6497, 7335, 8033, 8269) Selected clusters of enriched variants in the macaque CNS with conserved sequence properties. Individual residues are color-coded according to their functional properties to highlight conserved aspects of the sequence motif. The rank of each variant corresponds to the sum of the ranks of two synonymous DNA sequences.

FIG. 20A-20B—PAL family capsid variants and other macaque-derived variants outperform AAV9 and mouse- and marmoset-derived variants in transduction of a variety of macaque brain regions. (FIG. 20A) Fold difference in within-individual hFXN mRNA expression from different variants normalized to AAV9 in various central nervous system tissues of cynomolgus macaques. Dashed red line represents AAV9-CBh-hFXN expression normalized to 1. Data are represented as mean±SD (n=3); *p<0.05, **p<0.01 (one-way ANOVA with Dunnett's MCT and AAV9 as the control). (FIG. 20B) Quantification of transgene delivery efficiency, expressed as vector genomes per diploid genome, of different variants in the macaque liver. Data are represented as mean±SD (n=3); *p<0.05, **p<0.01 (one-way ANOVA with Dunnett's MCT and AAV9 as the control).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intra-arterial, intrathecal, lumbar, subdural, intracisternal, subpial, subretinal, subconjunctival, intravitreal, intratympanic, intracochlear, intradermal, intraventricular, intraosseous, intraocular, intracranial, intraperitoneal, intralesional, intranasal, intracardiac, intraarticular, intracavemous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavemous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide central nervous system (CNS)-specific targeting moieties that can be coupled to or otherwise associated with a cargo and/or delivery vehicle or system. Embodiments disclosed herein provide polypeptides (used interchangeably herein with the term “proteins”) and particles that can incorporate one or more of the CNS-specific targeting moieties. The polypeptides and/or particles can be coupled to, attached to, encapsulate, or otherwise incorporate a cargo, thereby associating the cargo with the targeting moiety(ies). Embodiments disclosed herein provide CNS-specific targeting moieties that contain one or more n-mer insert as further described herein. The targeting moieties may be used to provide engineered adeno-associated virus (AAV) capsids with a reprogrammed cell-specific and/or species-specific tropism, such as CNS specific tropism, to an engineered AAV particle.

In one example embodiment, the n-mer insert(s) is or contains a P-motif. In one example embodiment, the P-motif comprises the amino acid sequence XₘPX₁QGTX₂RXₙ (SEQ ID NO: 8580), wherein X₁, X₂, Xₘ, and Xₙ, are each independently selected from any amino acid, wherein m is 0, 1, 2, or 3, and wherein n is 0, 1, 2, 3, 4, 5, 6, or 7, and optionally a cargo, wherein the cargo is coupled to or is otherwise associated with the targeting moiety. In one example embodiment, the P-motif contains or is the amino acid sequence PX₁QGTX₂RXₙ (SEQ ID NO: 2), where X₁, X₂, Xₙ, are each selected from any amino acid and where n is 0, 1, 2, 3, 4, 5, 6, or 7.

In other example embodiments, the n-mer insert and/or P-motif is selected from the group consisting of SEQ ID NOs: 332-582 (Table 7).

In certain example embodiments, the targeting moiety comprises one or more n-mer inserts each comprising or consisting of a P-motif, wherein at least one of the P-motifs comprise the amino acid sequence XₘPX₁QGTX₂RXₙ (SEQ ID NO: 8580), wherein X₁, X₂, Xₘ, and Xₙ, are each independently selected from any amino acid, wherein m is 0, 1, 2, or 3, and wherein n is 0, 1, 2, 3, 4, 5, 6, or 7.

Embodiments disclosed herein also provide methods of generating recombinant AAVs (rAAVs) having engineered capsids that can involve systematically directing the generation of diverse libraries of variants of modified surface structures, such as variant capsid polypeptides. Embodiments of the method of generating rAAVs having engineered capsids can also include stringent selection of capsid variants capable of targeting CNS cells. As used in this context herein, “targeting” refers to the ability to, in a target specific manner, recognize, bind, associate with, transduce or infect, or otherwise interact with a target molecule or moiety such that recognition, binding, association, affinity, avidity, transduction or infection, and/or other interaction with the target molecule or moiety by the targeting moiety is greater, more efficient, or otherwise more selective for the target molecule or moiety as compared with its recognition, binding, association, affinity, avidity, transduction or infection, and/or other interaction with a non-target molecule or moiety. For example, a CNS-specific targeting moiety can have increased and/or more efficient or selective recognition, binding, association, affinity, avidity, transduction or infection, and/or other interaction of or with CNS cells as compared to non-CNS cells. In one example embodiment the n-mer may result in increased transduction of neurons of the CNS. Embodiments of the method of generating rAAVs having engineered capsids can include stringent selection of capsid variants capable of efficient and/or homogenous transduction in at least two or more species.

Embodiments disclosed herein provide vectors and systems thereof capable of producing an engineered AAV described herein.

Embodiments disclosed herein provide cells that can be capable of producing the engineered AAV particles described herein. In some embodiments, the cells include one or more vectors or system thereof described herein.

Embodiments disclosed herein provide engineered AAVs that can include an engineered capsid described herein. In some embodiments, the engineered AAV can include a cargo polynucleotide to be delivered to a cell. In some embodiments, the engineered AAV may be used to deliver gene therapies including encoding gene editing systems. In other embodiments, the engineered AAV may be used to deliver vaccines, such as DNA or mRNA vaccines.

Embodiments disclosed herein provide formulations that can contain an engineered AAV vector or system thereof, an engineered AAV capsid, engineered AAV particles including an engineered AAV capsid described herein, and/or an engineered cell described herein that contains an engineered AAV capsid, and/or an engineered AAV vector or system thereof. In some embodiments, the formulation can also include a pharmaceutically acceptable carrier. The formulations described herein can be delivered to a subject in need thereof or a cell.

Embodiments disclosed herein also provide kits that contain one or more of the one or more of the polypeptides, polynucleotides, vectors, engineered AAV capsids, engineered AAV particles, cells, or other components described herein and combinations thereof and pharmaceutical formulations described herein. In embodiments, one or more of the polypeptides, polynucleotides, vectors, engineered AAV capsids, engineered AAV particles cells, and combinations thereof described herein can be presented as a combination kit.

Embodiments disclosed herein provide methods of using the engineered AAVs having a cell-specific tropism described herein to deliver, for example, a therapeutic polynucleotide to a cell. In this way, the engineered AAVs described herein can be used to treat and/or prevent a disease in a subject in need thereof. Embodiments disclosed herein also provide methods of delivering the engineered AAV capsids, engineered AAV virus particles, engineered AAV vectors or systems thereof and/or formulations thereof to a cell. Also provided herein are methods of treating a subject in need thereof by delivering an engineered AAV particle, engineered AAV capsid, engineered AAV capsid vector or system thereof, an engineered cell, and/or formulation thereof to the subject.

Additional features and advantages of the embodiments engineered AAVs and methods of making and using the engineered AAVs are further described herein.

### CNS-Specific Targeting Moieties and Compositions

Generally, described herein are compositions containing one or more CNS-specific targeting moieties that can effectively target CNS cells. In some embodiments, the CNS-specific targeting moieties can be specific to one or more types of CNS cells. CNS cells include any cell within the brain, brain stem, spinal cord, inner ear, and eyes. In some embodiments, one or more CNS-specific targeting moieties can be incorporated into a delivery vehicle, agent, or system thereof so as to provide CNS specific targeting capability to the delivery vehicle, agent, or system thereof. Exemplary delivery vehicles include, without limitation, viral particles, (e.g., AAV viral particles), micelles, liposomes, exosomes, and the like. Exemplary delivery vehicles in which the CNS targeting-moieties can be incorporated are described in greater detail elsewhere herein. The CNS-targeting moieties may also be indirectly or directly coupled to a cargo and thus provide CNS specificity to the coupled cargo. In some embodiments, the composition can be specific for a CNS-cell (e.g., as conferred by the CNS-Specific targeting moieties described herein) and have reduced specificity for a non-CNS cell (including but not limited to a liver cell). In some embodiments, the CNS targeting moiety can specifically interact with or otherwise associate with one or more AAV receptors on CNS cells, thus providing CNS specificity (or tropism). Methods of generating and identifying CNS-specific targeting moieties are described in greater detail elsewhere herein.

### CNS-Specific Targeting Moieties

Described herein are targeting moieties capable of specifically targeting, binding, associating with, or otherwise interacting specifically with a CNS cell. In some embodiments, the targeting moiety effective to transduce, such as specifically transduce, a central nervous system (CNS) cell, comprises an n-mer insert optionally comprising or consisting of a P-motif, double valine motif, or both, and optionally a cargo, wherein the cargo is coupled to or is otherwise associated with the targeting moiety. Generally, n-mer inserts are short (e.g., about 3 to about 15, 20, or 25) amino acid sequences where each amino acid of the n-mer insert can be selected from any amino acid. In some embodiments, the n-mer insert is 3, 4, 5, 6, 7, 8, 9, 10, 11, 12, 13, 14, 15, 16, 17, 18, 19, 20, 21, 22, 23, 24, or 25 amino acids in length.

In certain example embodiments, where the targeting moiety comprises one or more n-mer inserts comprising or consisting of a P-motif, at least one of the P-motifs comprises or consists of the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7.

The term “P-motif” as used herein refers to an n-mer inserts that contains or is the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7. In some embodiments Xₘ is 2 and is AQ or DG. In some embodiments, the P-motif contains or is the amino acid sequence XₘPX₁QGTX₃RXₙ (SEQ ID NO: 8581), where X₁, X₃, Xₙ, are each selected from any amino acid, where m is 0, 1, 2, or 3, and where n is 0, 1, 2, 3, 4, 5, 6, or 7. In some embodiments, the P-motif contains or is the amino acid sequence PX₁QGTX₃RXₙ (SEQ ID NO: 2), where X₁, X₃, Xₙ, are each selected from any amino acid and where n is 0, 1, 2, 3, 4, 5, 6, or 7. n-mer inserts are described in greater detail elsewhere herein.

In certain example embodiments, the n-mer insert is or includes a double valine motif. As used herein the term “double valine motif” refers to an n-mer insert motif that has the amino acid sequence XₘX₁X₂VX₃X₄VX₅Xₙ, wherein X₁, X₂, X₃, Xₘ, and Xₙ, are each independently selected from any amino acid, wherein m is 0, 1, 2, or 3, and wherein n is 0, 1, 2, 3, 4, 5, 6, or 7.

In some embodiments, where an n-mer insert is or includes a P motif having the sequence amino acid sequence XₘPX₁X₂GTX₃RXₙ (SEQ ID NO: 8579) or XₘPX₁QGTX₃RXₙ (SEQ ID NO: 8581) or a double valine motif having the sequence XₘX₁X₂VX₃X₄VX₅Xₙ, and Xₘ in the P motif or double valine motif is not 0 (i.e., m=1, 2 or 3) the amino acids of Xₘ residues of the motif can replace up to 1, 2, or 3, respectively amino acids of the polypeptide into which the n-mer insert is being incorporated, such as a targeting moiety (e.g., a polypeptide, viral polypeptide, viral capsid polypeptide, and/or the like). Incorporation of an n-mer insert in this manner can position a P motif or double valine motif as an “insertion” between any two desired contiguous amino acids of the recipient polypeptide.

In some embodiments, the two amino acid residues immediately preceding the n-mer insert are AQ or DG in a targeting moiety or a composition that is a polypeptide. In some embodiments, where Xₘ is 0, the two amino acid residues in the targeting moiety immediately preceding the P-motif or double valine motif are AQ or DG.

In some embodiments, Xₙ of the P-motif or double valine motif is 0. In some embodiments, Xₙ of the P-motif or double valine motif is 1. In some embodiments, Xₙ of the P-motif or double valine motif is 2. In some embodiments, Xₙ of the P-motif or double valine motif is 3. In some embodiments, Xₙ of the P-motif or double valine motif is 4. In some embodiments, Xₙ of the P-motif or double valine motif is 5. In some embodiments, Xₙ of the P-motif or double valine motif is 6. In some embodiments, Xₙ of the P-motif or double valine motif is 7. In some embodiments, Xₘ of the P-motif or double valine motif is 0. In some embodiments, Xₘ of the P motif or double valine motif is 3. In some embodiments, Xₘ of the P motif or double valine motif is 2. In some embodiments, Xₘ of the P motif or double valine motif is 1.

In certain example embodiments, X₂ of the P motif is Q, P, E, or H. In certain example embodiments, X₁ of the P motif is a polar amino acid, optionally a polar uncharged amino acid. In certain example embodiments, X₃ of the P motif is a nonpolar amino acid. In certain example embodiments, X₁ of the P motif is S, T, N, Q, C, Y or A, X₂ of the P motif is Q, P, E, or H, X₃ is G, A, M, W, L, V, F, or I, or any combination thereof.

In certain example embodiments, X₁ of the double valine motif is R, K, V, or W. In certain example embodiments, X₂ of the double valine motif is T, S, V, Y or R. In certain example embodiments, X₃ of the double valine motif is G, P, or S. In certain example embodiments, X₄ of the double valine motif is S, D, or T. In certain example embodiments, X₅ of the double valine motif is Y, G, S, or L.

In some embodiments, Xₙ of the n-mer insert is 0. In some embodiments, the CNS-specific n-ner motif is as in any of Tables 1-3. In some embodiments, the CNS-specific n-mer insert is any one of the n-mer inserts in Table 6 (SEQ ID NOs.: 321-329). In some embodiments the CNS-specific n-mer insert is any one or more of the n-mer inserts selected from the group of SEQ ID NOs.: 322-324. In some embodiments the CNS-specific n-mer insert is any one or more of the n-mer inserts selected from the group of SEQ ID NOs.: 322-325. In some embodiments the CNS-specific n-mer insert is any one or more of the n-mer inserts selected from the group of SEQ ID NOs.: 322-327. In some embodiments the CNS-specific n-mer insert is any one or more of the n-mer inserts selected from the group of SEQ ID NOs.: 322-324 and 329. In some embodiments the CNS-specific n-mer insert and/or P-motif is any one or more of the n-mer inserts selected from the group of SEQ ID NOs.: 322-324. In some embodiments the CNS-specific n-mer insert any one or more of the n-mer inserts selected from the group of SEQ ID NOs.: 322-324 and 326-327. In some embodiments the CNS-specific n-mer insert is any one or more of the n-mer inserts selected from the group of SEQ ID NOs.: 322-324 and 326-328. In some embodiments the CNS-specific n-mer insert and is any one or more of the n-mer inserts selected from the group of SEQ ID NOs.: 322-324 and 328.

In certain example embodiments, at least one P-motif is selected from any one of SEQ ID NOs: 332-582 (Table 7).

In some embodiments, the n-mer insert(s) and/or at least one P-motif and/or double valine motif is selected from any one n-mer insert and/or is encoded by a polynucleotide as set forth in Table 8 (SEQ ID NOs: 583-8578). In some embodiments, the n-mer insert(s) and/or at least one P-motif and/or double valine motif is selected from any one n-mer insert and/or is encoded by a polynucleotide having a sequence according to any one of SEQ ID NOs: 583-2582. In some embodiments, the n-mer insert(s) and/or at least one P-motif and/or double valine motif is selected from any one n-mer insert and/or is encoded by a polynucleotide having a sequence according to any one of SEQ ID NOs: 2583-4582. In some embodiments, the n-mer insert(s) and/or at least one P-motif and/or double valine motif is selected from any one n-mer insert and/or is encoded by a polynucleotide having a sequence according to any one of SEQ ID NOs: 4583-6578. In some embodiments, the n-mer insert(s) and/or at least one P-motif and/or double valine motif is selected from any one n-mer insert and/or is encoded by a polynucleotide having a sequence according to any one of SEQ ID NOs: 6579-8578.

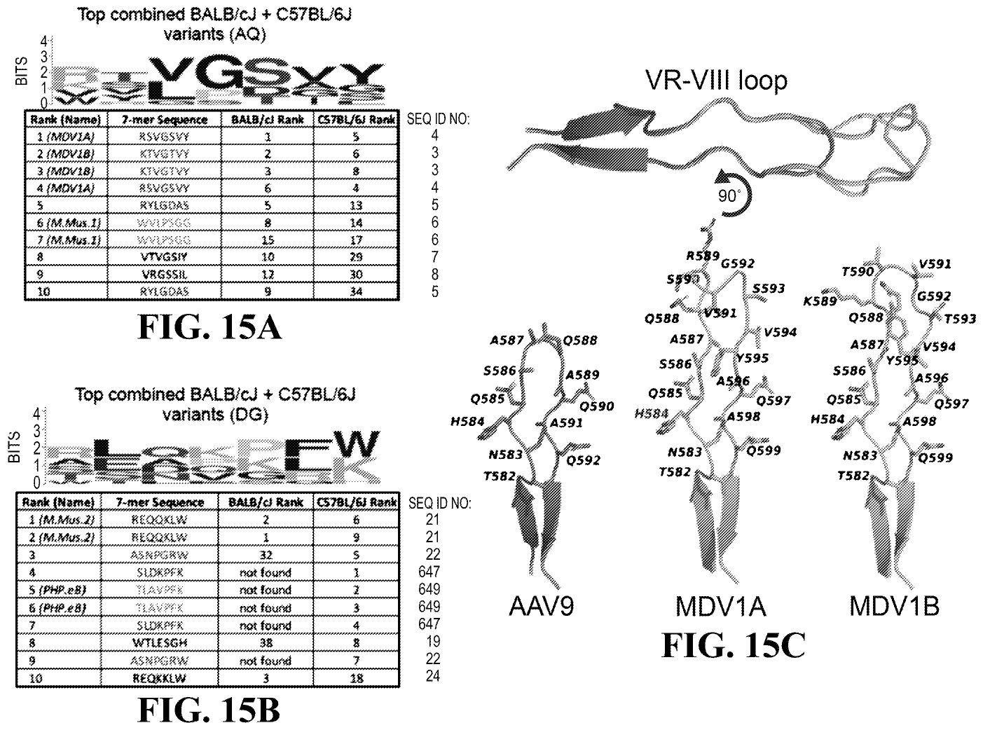

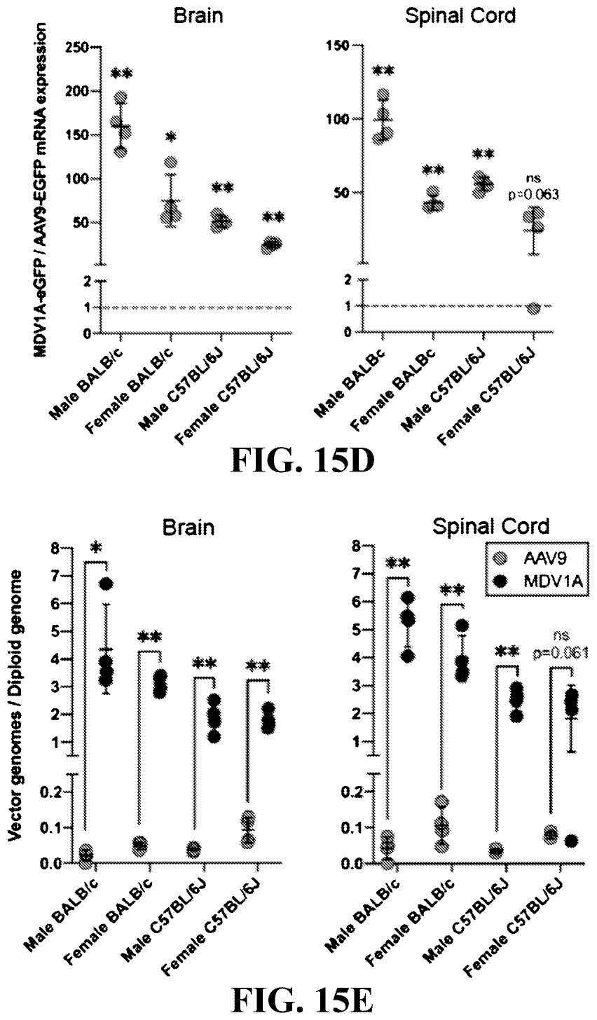

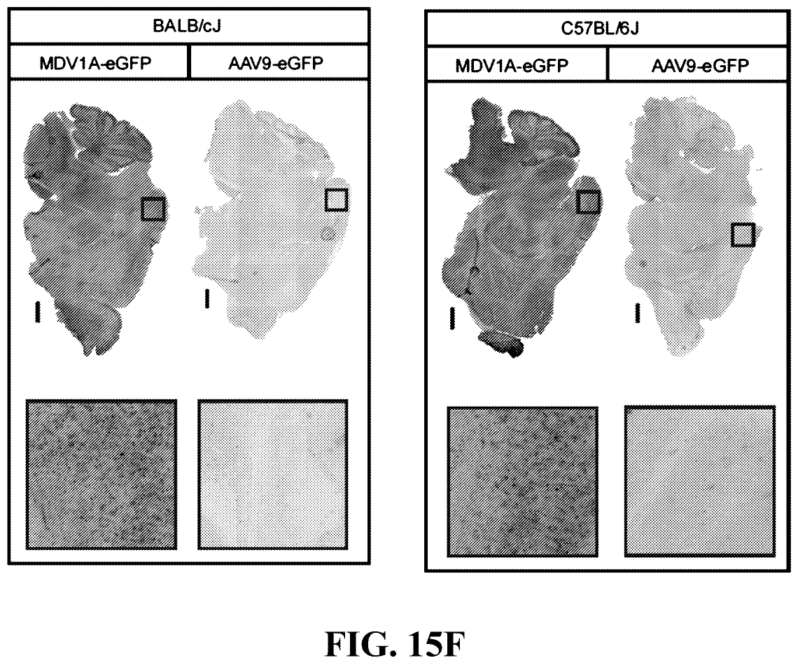

In certain example embodiments, the n-mer insert(s) and/or at least one P-motif and/or double valine motif is selected from any one n-mer insert and/or is encoded by a polynucleotide as set forth in one or more of SEQ ID NOs: 332-582 (Table 7), SEQ ID NOs: 583-8578 (Table 8), SEQ ID NOs: 3-819, 21-22, 24, 200, 202, 204, 212, 218, 224, 226, 228, 286, 234, 258, 260, 647, 649, 923, 1069, 1077, 1265, 2439, 2529, 2759, 3283, 3553, 3923, 4005, 4173, 4537, 4593, 4599, 4601, 4605, 4619, 4665, 4751, 4759, 4825, 4909, 4933, 5013, 5091, 5107, 5127, 5131, 5165, 5177, 5181, 5187, 5189, 5191, 5277, 5287, 5401, 5433, 5631, 5633, 5731, 5741, 5937, 6019, 6045, 6139, 6169, 6497, 7335, 8033, 8269, 8596-8613, (FIGS. 15A, 15B, 17A, 16A, 16B, 16C, and 19A-19C).

In some embodiments, the CNS-specific n-mer motif is and/or is encoded by a polynucleotide in FIG. 15A (SEQ ID NOs. 3-8).

In some embodiments, the CNS-specific n-mer motif is and/or is encoded by a polynucleotide in FIG. 15B (SEQ ID NOs. 19, 21-22, 24, 647, 649).

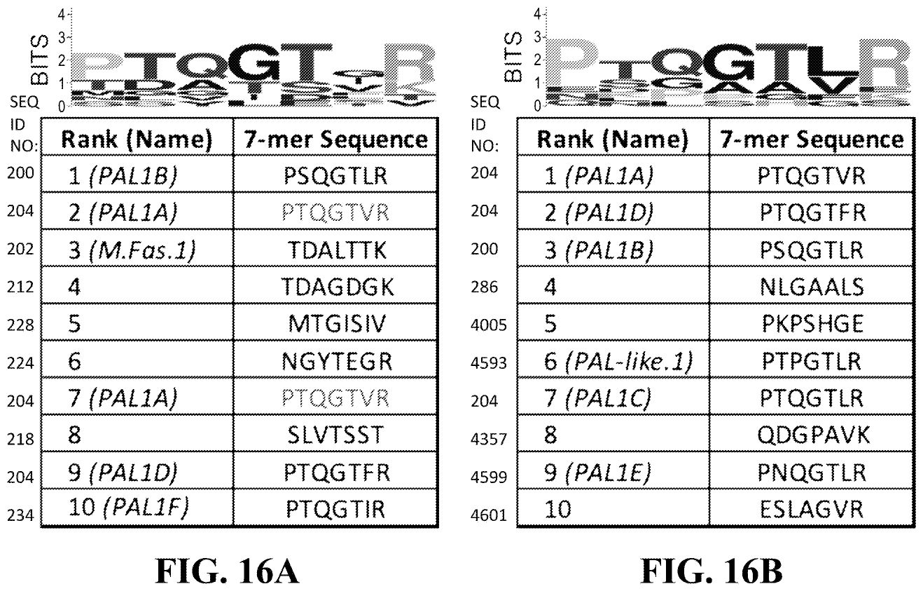

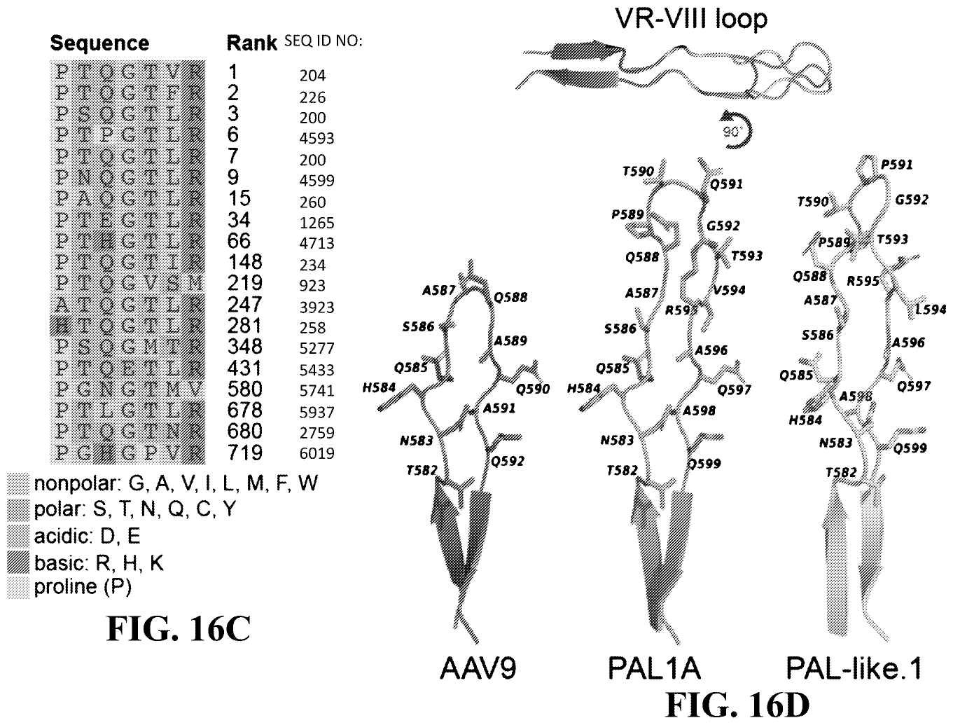

In some embodiments, the CNS-specific n-mer motif is and/or is encoded by a polynucleotide in FIG. 16A (SEQ ID NOs. 200, 202, 204, 212, 218, 224, 228, 234).

In some embodiments, the CNS-specific n-mer motif is and/or is encoded by a polynucleotide in FIG. 16B (SEQ ID NOs. 200, 204, 286, 4005, 4537, 4593, 4599, 4601).

In some embodiments, the CNS-specific n-mer motif is and/or is encoded by a polynucleotide in FIG. 16C (SEQ ID NOs. 200, 204, 226, 234, 258, 260, 923, 1265, 2759, 3923, 4173, 4593, 4599, 5277, 5433, 5741, 5937, 6019).

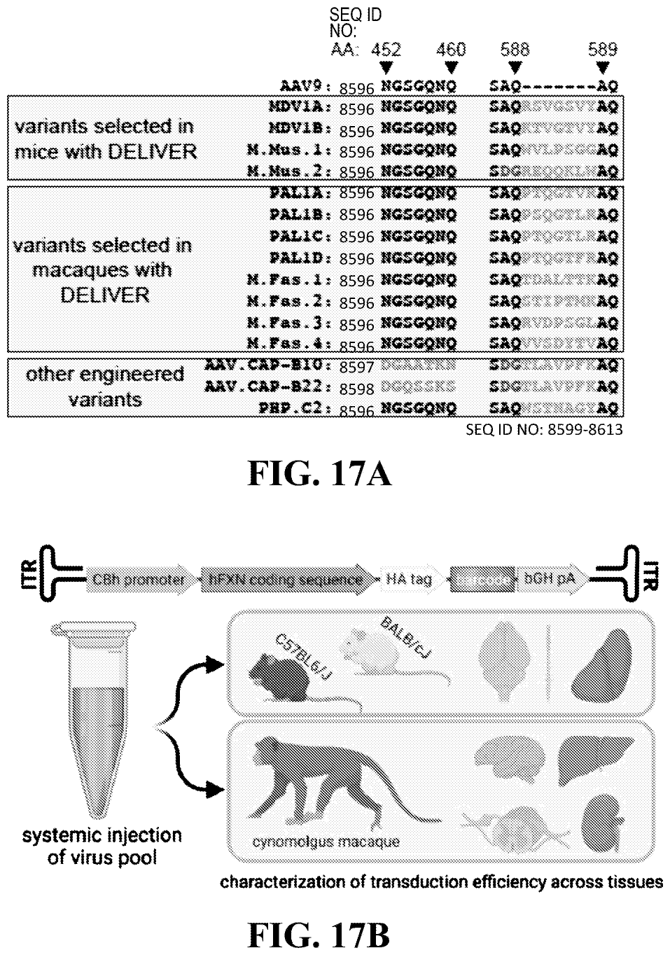

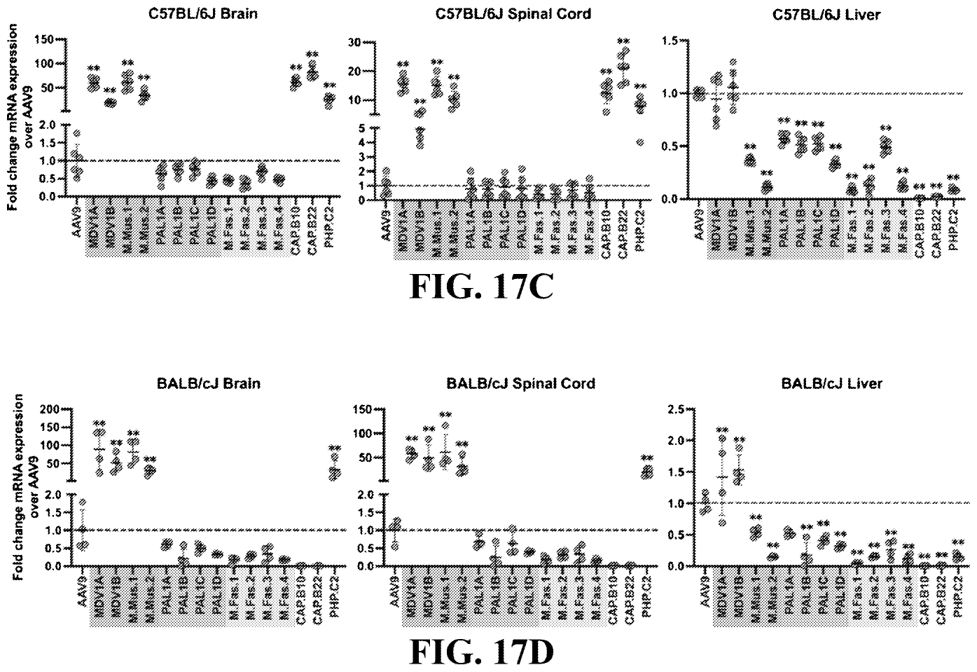

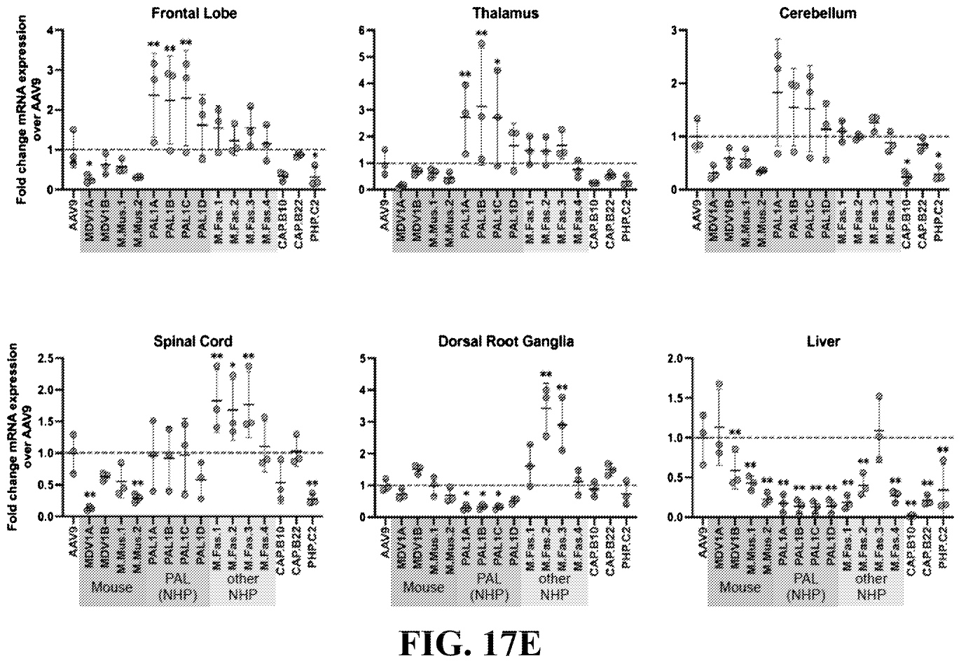

In some embodiments, the CNS-specific n-mer motif is and/or is encoded by a polynucleotide in FIG. 17A (SEQ ID NOs. 8596-8613).

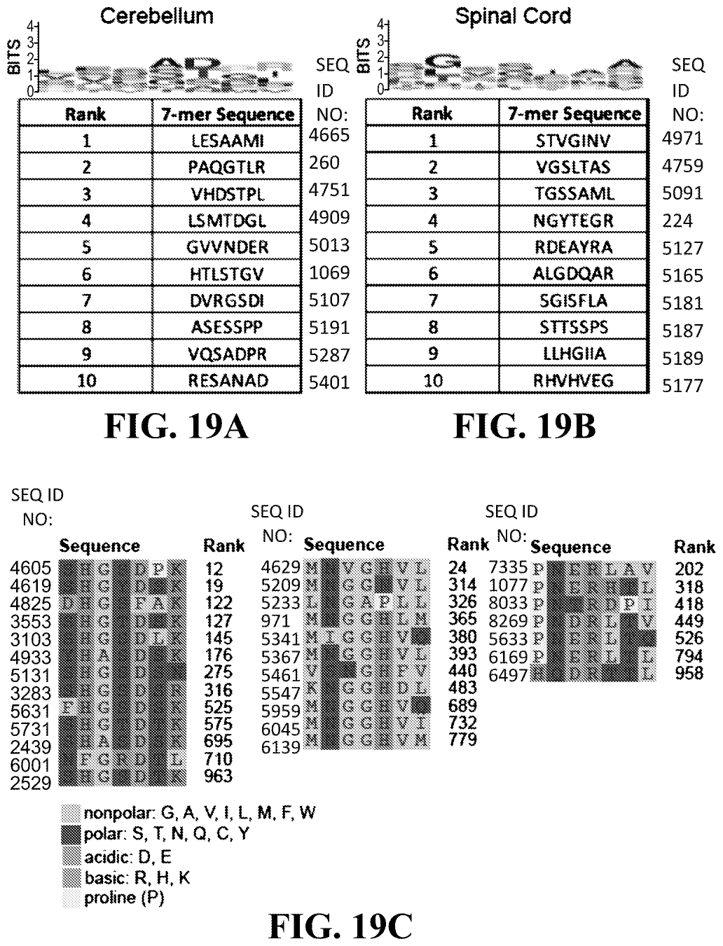

In some embodiments, the CNS-specific n-mer motif is and/or is encoded by a polynucleotide in FIG. 19A (SEQ ID NOs. 260, 1069, 4665, 4751, 4909, 5013, 5107, 5191, 5287, 5401).

In some embodiments, the CNS-specific n-mer motif is and/or is encoded by a polynucleotide in FIG. 19B (SEQ ID NOs. 224, 4759, 4971, 5091, 5127, 5165, 5177, 5181, 5187, 5189).

In some embodiments, the CNS-specific n-mer motif is and/or is encoded by a polynucleotide in FIG. 19C (SEQ ID NOs: 2439, 2529, 3103, 3283, 3553, 4605, 4619, 4825, 4933, 5131, 5631, 5731, 6001, 971, 4629, 5209, 5233, 5341, 5367, 5461, 5547, 5959, 6045, 6139, 1077, 7335, 8033, 8269, 5633, 6169, 6497). In some embodiments, the CNS-specific n-mer motif is and/or is encoded by a polynucleotide having a sequence according to any one of SEQ ID NOs: 2439, 2529, 3103, 3283, 3553, 4605, 4619, 4825, 4933, 5131, 5631, 5731, 6001. In some embodiments, the CNS-specific n-mer motif is and/or is encoded by a polynucleotide having a sequence according to any one of SEQ ID NOs: 971, 4629, 5209, 5233, 5341, 5367, 5461, 5547, 5959, 6045, 6139. In some embodiments, the CNS-specific n-mer motif is and/or is encoded by a polynucleotide having a sequence according to any one of SEQ ID NOs: 1077, 7335, 8033, 8269, 5633, 6169, 6497.

In some embodiments, the CNS-specific n-mer insert is species specific. In other words, in some embodiments, the CNS-specific n-mer insert can facilitate CNS targeting in one species better than another species. In some embodiments the CNS-specific n-mer insert is specific for primates. In some embodiments, the CNS-specific n-mer insert is specific for human and/or non-human primates.

In some embodiments, the CNS-specific n-mer insert is capable of targeting one or more cell and/or tissue types over others within the CNS. In some embodiments, the CNS-specific insert is not effective or is less effective at targeting the dorsal root ganglion cells than one or more other cells and/or tissue types of the CNS.

In some embodiments, the CNS-specific n-mer insert is capable of targeting a specific CNS tissue type or cell type. In some embodiments, the CNS-specific n-mer insert is capable of targeting one or more specific regions of the CNS as set forth in Table 9. n some embodiments, the CNS-specific n-mer insert is capable of targeting the frontal lobe, the temporal lobe or specific region thereof (e.g., the posterior or anterior temporal lobe), the parietal lobe or specific region thereof (e.g., the posterior or anterior parietal lobe), the occipital lobe the thalamus, the corpus callosum, the cerebellum, neuroretina, RPE, brain stem, the spinal cord or a region therein (e.g., the cervical spinal cord, the thoracic spinal cord, the lumbar spinal cord), cauda equina, DRGs or subset thereof (e.g., cervical DRG, thoracic DRG, lumbar DRG), or any combination thereof.

In some embodiments, the targeting moiety can include more than one n-mer inserts, such as a CNS-specific n-mer insert described herein. In some embodiments, the targeting moiety can include 1, 2, 3, 4, 5, 6, 7, 8, 9, 10 or more n-mer inserts. In some embodiments, all the n-motifs included in the targeting moiety can be the same. In some embodiments where more than one n-mer insert is included, at least two of the n-mer inserts are different from each other. In some embodiments where more than one n-mer insert is included, all the n-mer inserts are different from each other.

In one example embodiment, the targeting moiety, e.g., the CNS-specific targeting moiety, can be coupled to or otherwise associated with a cargo. In some embodiments, one or more CNS-specific targeting moieties described herein is directly attached to the cargo. In some embodiments, one or more CNS-specific targeting moieties described herein is indirectly coupled to the cargo, such as via a linker molecule.

In another example embodiment, one or more CNS-specific targeting moieties described herein is coupled to associated with a particle that is coupled to, attached to, encapsulates, and/or contains a cargo. Exemplary particles include, without limitation, viral particles (e.g., viral capsids, which is inclusive of bacteriophage capsids), polysomes, liposomes, nanoparticles, microparticles, exosomes, micelles, and the like. The term “nanoparticle” as used herein includes a nanoscale deposit of a homogenous or heterogeneous material. Nanoparticles may be regular or irregular in shape and may be formed from a plurality of co-deposited particles that form a composite nanoscale particle. Nanoparticles may be generally spherical in shape or have a composite shape formed from a plurality of co-deposited generally spherical particles. Exemplary shapes for the nanoparticles include, but are not limited to, spherical, rod, elliptical, cylindrical, disc, and the like. In some embodiments, the nanoparticles have a substantially spherical shape.

As used herein, the term “specific” when used in relation to described an interaction between two moieties, refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

In some embodiments, in addition to the n-mer insert(s) the targeting moiety can include a polypeptide, a polynucleotide, a lipid, a polymer, a sugar, or a combination thereof.

In some embodiments, the targeting moiety is incorporated into a viral polypeptide, such as a capsid polypeptide, including but not limited to lentiviral, adenoviral, AAV, bacteriophage, and retroviral polypeptides. In some embodiments, the n-mer insert is inserted between two amino acids of the viral polypeptide such that the n-mer insert is external (i.e., is presented on the surface of) to a viral capsid.

In some embodiments, the composition containing one or more of the CNS-specific targeting moieties described herein has increased muscle cell potency, muscle cell specificity, reduced immunogenicity, or any combination thereof.

Cargos can include any molecule that is capable of being coupled to or associated with the CNS-specific targeting moieties described herein. Cargos can include, without limitation, nucleotides, oligonucleotides, polynucleotides, amino acids, peptides, polypeptides, riboproteins, lipids, sugars, pharmaceutically active agents (e.g., drugs, imaging and other diagnostic agents, and the like), chemical compounds, and combinations thereof. In some embodiments, the cargo is DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, radiation sensitizers, chemotherapeutics, radioactive compounds, imaging agents, and combinations thereof.

The CNS-specific targeting moieties can be encoded in whole or in part by a polynucleotide. The encoding polynucleotides can be included in one or more vectors (or vector systems) that can be used to generate targeting moieties and compositions thereof that include the CNS-specific n-mer insert(s) Exemplary encoding polynucleotides, vectors, vector systems, and recombinant engineering techniques are described in greater detail herein and/or are generally known in the art and can be adapted for use with the targeting moieties and compositions thereof described herein.

In some embodiments, the cargo is capable of treating or preventing a CNS disease or disorder. Exemplary CNS diseases and disorders are described elsewhere herein.

### Cargos

Representative cargo molecules that may be delivered using the compositions disclosed herein include, but are not limited to, nucleic acids, polynucleotides, proteins, polypeptides, polynucleotide/polypeptide complexes, small molecules, sugars, or a combination thereof. Cargos that can be delivered in accordance with the systems and methods described herein include, but are not necessarily limited to, biologically active agents, including, but not limited to, therapeutic agents, imaging agents, and monitoring agents. A cargo may be an exogenous material or an endogenous material. In some embodiments, the cargo can be a “gene of interest”.

In some embodiments the cargos, in addition to the cargo of interest that is to be delivered to a CNS cell, the cargo contains one or more binding sites specific for one or more RNAi molecules that are endogenous to one or more non-target (such as non-CNS cells). In this context herein “non-target cells” refers to cells to which delivery or activity of a cargo is not desired. In other words, “non-target cells” are cells in which the targeting moiety, such as the CNS specific targeting moiety, and compositions thereof do not specifically target. When a cargo having one more specific binding sites for one or more RNAi molecules that are endogenous to one or more non-target cells is delivered to non-target cells, the endogenous RNAi molecule of the non-target cell degrades the cargo molecule via the endogenous RNAi pathway. In this way off-target toxicity or other deleterious off-target events can be reduced. This can also be referred to as a mechanism of detargeting the composition to non-target cells.

In some embodiments, the detargeting component of a cargo molecule is one or more specific binding sites for one or more RNAi molecules that are endogenous to one or more non-target cells. In some embodiments, the RNAi molecules that are endogenous to one or more non-target cells are specifically expressed in those non-target cell(s). In some embodiments, the RNAi molecules that are endogenous to one or more non-target cells are enriched or have greater expression in non-target cell(s) as compared to target cells, such as CNS cells. In some embodiments, the more RNAi molecules that are endogenous to one or more non-target cells are not expressed in a target cell, such as a CNS cell. Exemplary RNAi molecule types are described elsewhere herein. In some embodiments, the one or more RNAi molecules that are endogenous to one or more non-target cells are microRNAs. In some embodiments, the non-target cell(s) are liver cell(s) and/or dorsal root ganglion neuron(s). In some embodiments, the RNAi molecules are miR183, miR-182, miR122, miR122a, miR99a, miR-26a, miR199a, miRNA-143, miR101a, miR-30c, or any combination thereof.

Other exemplary detargeting RNAi molecules are described in e.g., International Patent Application Pub. WO2021231579A1 and WO2020132455A1, https://www-hebertpub-com.ezp-prod1.hul.harvard.edu/doi/pdf/10.1089%2Fnat.2015.0543.

### Polynucleotides

In some embodiments, the cargo is a cargo polynucleotide. As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically, or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA, including but not limited to, tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), or coding mRNA (messenger RNA).

In some embodiments, the cargo polynucleotide is DNA. In some embodiments, the cargo polynucleotide is RNA. In some embodiments, the cargo polynucleotide is a polynucleotide (a DNA or an RNA) that encodes an RNA and/or a polypeptide. As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

### Genes of Interest

In some embodiments, the systems described herein comprise a polynucleotide encoding a gene of interest. As used herein, the term “gene of interest” refers to the gene selected for a particular purpose and being desired of delivery by a system or vesicle of the present invention. A gene of interest inserted into one or more regions a vector, such as an expression vector (including one or more of the engineered delivery vesicle generation system vectors) such that when expressed in a target cell or recipient cell it can be expressed and produce a desired gene product and/or be packaged as cargo in an engineered delivery vesicle of the present invention. It will be appreciated that other cargos specifically identified can also be genes of interest. For example, a polynucleotide encoding a Cas effector can be a gene of interest in this context where it is desired to deliver a Cas effector to a cell, for example.

In one embodiment, the gene of interest encodes a gene that provides a therapeutic function for the treatment of a disease. In some embodiments, the gene of interest can also be a vaccinating gene, that is to say a gene encoding an antigenic peptide that is capable of generating an immune response in humans or animals. This may include, but is not necessarily limited to, peptide antigens specific for viral and bacterial infections, or may be tumor-specific. In some embodiments, a gene of interest is a gene which confers a desired phenotype. As the embodiments described herein focus on improved methods for packaging and delivery of a gene of interest, the particular gene of interest is not limiting and the technology can generally be used to deliver any gene of interest generally recognized by one of ordinary skill in the art as deliverable using a lentiviral system. One skilled in the art can design a construct containing any gene that they are interested in. Designing a construct containing a known gene of interest can be performed without undue experimentation. One of ordinary skill in the art routinely selects genes of interest. For example, the GenBank public database has existed since 1982 and is routinely used by persons of ordinary skill in the art relevant to the presently claimed method. As of June 2019, GenBank contains 2013,383,758 loci, 329,835,282,370 bases, from 213,383,758 reported sequences. The nucleotide sequences are from more than 300,000 organisms with supporting bibliographic and biological annotation. GenBank is only example, as there are many other known repositories of sequence information.

In some embodiments, the gene of interest may be, for example, a synthetic RNA/DNA sequence, a codon optimized RNA/DNA sequence, a recombinant RNA/DNA sequence (i.e., prepared by use of recombinant DNA techniques), a cDNA sequence or a partial genomic DNA sequence, including combinations thereof. Preferably, this is in the sense orientation. Preferably, the sequence is, comprises, or is transcribed from cDNA. The gene(s) of interest may also be referred to herein as “heterologous sequence(s)” “heterologous gene(s)” or “transgene(s)”.

In some embodiments, the gene of interest may confer some therapeutic benefit. The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

Preferably, the therapeutic agent may be administered in a therapeutically effective amount of the active components. The term “therapeutically effective amount” refers to an amount which can elicit a biological or medicinal response in a tissue, system, animal, or human that is being sought by a researcher, veterinarian, medical doctor or other clinician, and in particular can prevent or alleviate one or more of the local or systemic symptoms or features of a disease or condition being treated. In some embodiments, the disease or condition is a disease or condition of or affecting the CNS or cell thereof. Exemplary diseases and disorders of and/or affecting the CNS are described in greater detail elsewhere herein.

In some embodiments, the gene of interest may lead to altered expression in the target cell. As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

Also, “altered expression” as intended herein may encompass modulating the activity of one or more endogenous gene products. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar. As used herein, “modulating” or “to modulate” generally means either reducing or inhibiting the activity of a target or antigen, or alternatively increasing the activity of the target or antigen, as measured using a suitable in vitro, cellular, or in vivo assay. In particular, “modulating” or “to modulate” can mean either reducing or inhibiting the (relevant or intended) activity of, or alternatively increasing the (relevant or intended) biological activity of the target or antigen, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

### Interference RNAs

In certain example embodiments, the one or more polynucleotides, such as cargo polynucleotides, may encode one or more interference RNAs. Interference RNAs are RNA molecules capable of suppressing gene expressions. Example types of interference RNAs include small interfering RNA (siRNA), micro RNA (miRNA), and short hairpin RNA (shRNA). It will be appreciated that a cargo can include an RNAi molecule to be delivered to a target cell as well as a binding site for an endogenous RNAi molecule of a non-target cell. RNAi molecules that are to be delivered to a target cell as cargo can be e.g., therapeutic.

In certain example embodiments, the interference RNA may be a siRNAs. Small interfering RNA (siRNA) molecules are capable of inhibiting target gene expression by interfering RNA. siRNAs may be chemically synthesized, or may be obtained by in vitro transcription, or may be synthesized in vivo in target cell. siRNAs may comprise double-stranded RNA from 15 to 40 nucleotides in length and can contain a protuberant region 3′ and/or 5′ from 1 to 6 nucleotides in length. Length of protuberant region is independent from total length of siRNA molecule. siRNAs may act by post-transcriptional degradation or silencing of target messenger. In some cases, the exogenous polynucleotides encode shRNAs. In shRNAs, the antiparallel strands that form siRNA are connected by a loop or hairpin region.

The RNAi molecules delivered as cargo can, in some embodiments, suppress expression of genes and/or degrade a gene product (e.g., a transcript) related to a CNS disease, eye disease, or inner ear disease. Therefore, in some embodiments, the RNAi cargo treats or prevents a CNS disease, eye disease, or inner ear disease or symptom thereof.

The interference RNA (e.g., siRNA) may suppress expression of genes to promote long term survival and functionality of cells after transplanted to a subject. In some examples, the interference RNAs suppress genes in TGFβ pathway, e.g., TGFβ, TGFβ receptors, and SMAD proteins. In some examples, the interference RNAs suppress genes in colony-stimulating factor 1 (CSF1) pathway, e.g., CSF1 and CSF1 receptors. In certain embodiments, the one or more interference RNAs suppress genes in both the CSF1 pathway and the TGFβ pathway. TGFβ pathway genes may comprise one or more of ACVR1, ACVR1C, ACVR2A, ACVR2B, ACVRL1, AMH, AMHR2, BMP2, BMP4, BMP5, BMP6, BMP7, BMP8A, BMP8B, BMPR1A, BMPR1B, BMPR2, CDKN2B, CHRD, COMP, CREBBP, CUL1, DCN, E2F4, E2F5, EP300, FST, GDF5, GDF6, GDF7, ID1, ID2, ID3, ID4, IFNG, INHBA, INHBB, INHBC, INHBE, LEFTY1, LEFTY2, LOC728622, LTBP1, MAPK1, MAPK3, MYC, NODAL, NOG, PITX2, PPP2CA, PPP2CB, PPP2R1A, PPP2R1B, RBL1, RBL2, RBX1, RHOA, ROCK1, ROCK2, RPS6KB1, RPS6KB2, SKP1, SMAD1, SMAD2, SMAD3, SMAD4, SMAD5, SMAD6, SMAD7, SMAD9, SMURF1, SMURF2, SP1, TFDP1, TGFB1, TGFB2, TGFB3, TGFBR1, TGFBR2, THBS1, THBS2, THBS3, THBS4, TNF, ZFYVE16, and/or ZFYVE9.

In some embodiments, the cargo polynucleotide is an RNAi molecule, antisense molecule, and/or a gene silencing oligonucleotide or a polynucleotide that encodes an RNAi molecule, antisense molecule, and/or gene silencing oligonucleotide.

As used herein, “gene silencing oligonucleotide” refers to any oligonucleotide that can alone or with other gene silencing oligonucleotides utilize a cell's endogenous mechanisms, molecules, proteins, enzymes, and/or other cell machinery or exogenous molecule, agent, protein, enzyme, and/or polynucleotide to cause a global or specific reduction or elimination in gene expression, RNA level(s), RNA translation, RNA transcription, that can lead to a reduction or effective loss of a protein expression and/or function of a non-coding RNA as compared to wild-type or a suitable control. This is synonymous with the phrase “gene knockdown” Reduction in gene expression, RNA level(s), RNA translation, RNA transcription, and/or protein expression can range from about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, to 1% or less reduction. “Gene silencing oligonucleotides” include, but are not limited to, any antisense oligonucleotide, ribozyme, any oligonucleotide (single or double stranded) used to stimulate the RNA interference (RNAi) pathway in a cell (collectively RNAi oligonucleotides), small interfering RNA (siRNA), microRNA, and short-hairpin RNA (shRNA). Commercially available programs and tools are available to design the nucleotide sequence of gene silencing oligonucleotides for a desired gene, based on the gene sequence and other information available to one of ordinary skill in the art.

### Therapeutic Polynucleotides

In some embodiments, the cargo molecule is a therapeutic polynucleotide. Therapeutic polynucleotides are those that provide a therapeutic effect when delivered to a recipient cell. The polynucleotide can be a toxic polynucleotide (a polynucleotide that when transcribed or translated results in the death of the cell) or polynucleotide that encodes a lytic peptide or protein. In embodiments, delivery vesicles having a toxic polynucleotide as a cargo molecule can act as an antimicrobial or antibiotic. This is discussed in greater detail elsewhere herein. In some embodiments, the cargo molecule can be exogenous to the producer cell and/or a first cell. In some embodiments, the cargo molecule can be endogenous to the producer cell and/or a first cell. In some embodiments, the cargo molecule can be exogenous to the recipient cell and/or a second cell. In some embodiments, the cargo molecule can be endogenous to the recipient cell and/or second cell.

As described herein the cargo polynucleotide can be any polynucleotide endogenous or exogenous to the eukaryotic cell. For example, the cargo polynucleotide can be a polynucleotide residing in the nucleus of the eukaryotic cell. The cargo polynucleotide can be a sequence coding a gene product (e.g., a protein) or a non-coding sequence (e.g., a regulatory polynucleotide).

In some embodiments, the cargo polynucleotide is a DNA or RNA (e.g., a mRNA) vaccine.

### Aptamers

In certain example embodiments, the polynucleotide may be an aptamer. In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues, and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH2), 2′-fluoro (2′-F), and/or 2′-O-methyl (2′-OMe) substituents. Modifications of aptamers may also include modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, 0-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to, those described in Rohloffet al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see e.g., SomaLogic, Inc., Boulder, Colorado). In certain embodiments, the present invention may utilize any aptamer containing any modification as described herein.

In certain other example embodiments, the polynucleotide may be a ribozyme or other enzymatically active polynucleotide.

### Biologically Active Agents

In some embodiments, the cargo is a biologically active agent. Biologically active agents include any molecule that induces, directly or indirectly, an effect in a cell. Biologically active agents may be a protein, a nucleic acid, a small molecule, a carbohydrate, and a lipid. When the cargo is or comprises a nucleic acid, the nucleic acid may be a separate entity from the DNA-based carrier. In these embodiments, the DNA-based carrier is not itself the cargo. In other embodiments, the DNA-based carrier may itself comprise a nucleic acid cargo. Therapeutic agents include, without limitation, chemotherapeutic agents, anti-oncogenic agents, anti-angiogenic agents, tumor suppressor agents, anti-microbial agents, enzyme replacement agents, gene expression modulating agents and expression constructs comprising a nucleic acid encoding a therapeutic protein or nucleic acid, and vaccines. Therapeutic agents may be peptides, proteins (including enzymes, antibodies and peptidic hormones), ligands of cytoskeleton, nucleic acid, small molecules, non-peptidic hormones and the like. To increase affinity for the nucleus, agents may be conjugated to a nuclear localization sequence. Nucleic acids that may be delivered by the method of the invention include synthetic and natural nucleic acid material, including DNA, RNA, transposon DNA, antisense nucleic acids, dsRNA, siRNAs, transcription RNA, messenger RNA, ribosomal RNA, small nucleolar RNA, microRNA, ribozymes, plasmids, expression constructs, etc.

Imaging agents include contrast agents, such as ferrofluid-based MRI contrast agents and gadolinium agents for PET scans, fluorescein isothiocyanate and 6-TAMARA. Monitoring agents include reporter probes, biosensors, green fluorescent protein, and the like. Reporter probes include photo-emitting compounds, such as phosphors, radioactive moieties, and fluorescent moieties, such as rare earth chelates (e.g., europium chelates), Texas Red, rhodamine, fluorescein, FITC, fluor-3, 5 hexadecanoyl fluorescein, Cy2, fluor X, Cy3, Cy3.5, Cy5, Cy5.5, Cy7, dansyl, phycocrytherin, phycocyanin, spectrum orange, spectrum green, and/or derivatives of any one or more of the above. Biosensors are molecules that detect and transmit information regarding a physiological change or process, for instance, by detecting the presence or change in the presence of a chemical. The information obtained by the biosensor typically activates a signal that is detected with a transducer. The transducer typically converts the biological response into an electrical signal. Examples of biosensors include enzymes, antibodies, DNA, receptors, and regulator proteins used as recognition elements, which can be used either in whole cells or isolated and used independently (D'Souza, 2001, Biosensors and Bioelectronics 16:337-353).

One or two or more different cargoes may be delivered by the delivery particles described herein.

In some embodiments, the cargo may be linked to one or more envelope proteins by a linker, as described elsewhere herein. A suitable linker may include, but is not necessarily limited to, a glycine-serine linker. In some embodiments, the glycine-serine linker is (GGS)₃ (SEQ ID NO: 27).

In some embodiments, the cargo comprises a ribonucleoprotein. In specific embodiments, the cargo comprises a genetic modulating agent.

As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

### Genetic Modifying Systems

In some embodiments, the cargo is a polynucleotide encoding a gene modifying system. Gene modifying systems may include, but are not limited to, zinc finger nucleases, TALE nucleases (TALENs), meganucleases, RNAi, and CRISPR-Cas systems. The generic modifying systems can, upon delivery as cargo to a target cell, such as a CNS cell, result in a genetic modification in that cell. In some embodiments, the genetic modification cures, treats, and/or prevents a disease or disorder, such as a CNS, eye, or inner ear disease or disorder.

### CRISPR-Cas Systems

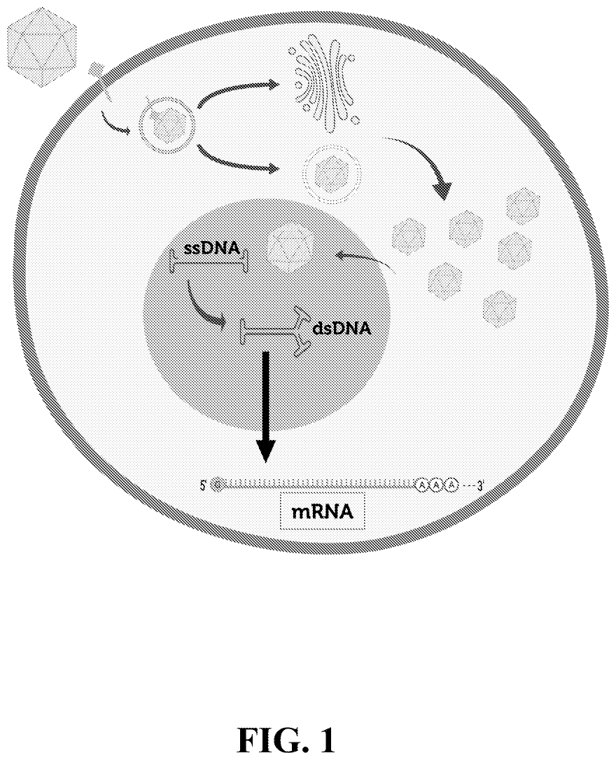

The CRISPR-Cas system may include a Class 1 comprising a Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), and incorporated in its entirety herein by reference, and particularly as described in FIG. 1, p. 326. polynucleotide modifying system or component(s) thereof. The CRISPR-Cas system may also be a Class 2 CRISPR-Cas system such as a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference.

CRISPR-Cas systems may also include further modified systems where the Cas protein is rendered catalytically inactive and fused to other functional domains or polypeptides to derive new functions. Example modified systems include base editor, primer editors, and CRISPR-associated transposase (CAST) systems.

Example base editing systems include DNA base editors (Komor et al. 2016 Nature. 533:420-424; Nishida et a. 2016. Science 353; Gaudelli et al. 2017 Nature 551:464-471; Mok et al., Cell. 182, 463-480 (2020); Koblan et al., Nature 589, 608-614 (2021); Rees and Liu. 2018. 19(12):770-788. doi: 10.1038/s41576-018-0059-1; Song et al., Nat Biomed Eng. 2020 Jan; 4(1):125-130. doi: 10.1038/s41551-019-0357-8; Koblan et al. 2018. 6(9):843-846. doi: 10.1038/nbt.4172; Thuronyi et al., Nat Biotechnol. 2019 September; 37(9):1070-1079. doi: 10.1038/s41587-019-0193-0; Doman et al., Nat Biotechnol. 2020 May; 38(5):620-628. doi: 10.1038/s41587-020-0414-6; Richter et al., Nat Biotechnol. 2020 July; 38(7):883-891. doi: 10.1038/s41587-020-0453-z; Huang et al., Nat Protoc. 2021 February; 16(2):1089-1128. doi: 10.1038/s41596-020-00450-9; Koblan et al., Nat Biotechnol. 2021 Jun. 28. doi: 10.1038/s41587-021-00938-z; WO 2018/213708, WO 2018/213726, WO/2019/126709, WO/2019/1267; WO/2019/126762) and RNA base editors (Cox et al. 2017. Science 358:1019-1027, Rees and Liu. 2018. 19(12):770-788. doi: 10.1038/s41576-018-0059-1; Abudayyeh 00, et al., A cytosine deaminase for programmable single-base RNA editing, Science 26 Jul. 2019; WO 2019/005883, WO 2019/005886, WO 2019/071048, PCT/US2018/0579, PCT US/2018/067207).

Example prime editing systems include those as described in Anzalone et al. 2019 Nature 576:149-157; Gao et al. 2021 Genome Biol. 22:83; Jang et al. 2021 Nature Biomed. Eng. doi.org/10.1038/s41551-021-00788-9; WO 2021/072328; WO 2020/191248; WO 2020/191249; WO 2020/191239; WO 2020/191245; WO 2020/191246; WO 2020/191241; WO 2020/191171; WO 202/191153; WO 2020/191242; WO 2020/191233; WO 2020/191243; and WO 2020/191234.

Example CAST systems include those as described in Klompe et al. 2019 Nature 571(7764):219-225; Strecker et al. 2019 Science 365:48-53; and Saito et al. 2021 Cell 184:2441-2453; WO 2020/131862; WO 2019090173; WO 2019090174; WO 2019090175, and WO 2019/241452.

Example non-LTR retrotransposon systems include those as described in WO2021/102042.

Example Cas-associated ligase systems include those as described in WO2021/133977.

For modified CRISPR-Cas system that exceed the cargo capacity for a delivery vehicle incorporating the targeting moieties disclosed herein, a split-intein approach to divide CBE and ABE into reconstitutable halves, is described in Levy et al. Nature Biomedical Engineering doi.org/10.1038/s41441-019-0505-5 (2019), which is incorporated herein by reference.

### Zinc Finger Nucleases

Zinc Finger proteins can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNA

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Polypeptides

In certain example embodiments, the cargo molecule may one or more polypeptides. The polypeptide may be a full-length protein or a functional fragment or functional domain thereof, that is a fragment or domain that maintains the desired functionality of the full-length protein. As used within this section “protein” is meant to refer to full-length proteins and functional fragments and domains thereof. A wide array of polypeptides may be delivered using the engineered delivery vesicles described herein, including but not limited to, secretory proteins, immunomodulatory proteins, anti-fibrotic proteins, proteins that promote tissue regeneration and/or transplant survival functions, hormones, anti-microbial proteins, anti-fibrillating polypeptides, and antibodies. The one or more polypeptides may also comprise combinations of the aforementioned example classes of polypeptides. It will be appreciated that any of the polypeptides described herein can also be delivered via the engineered delivery vesicles and systems described herein via delivery of the corresponding encoding polynucleotide.

### Secretory Proteins

In certain example embodiments, the one or more polypeptides may comprise one or more secretory proteins. A secretory is a protein that is actively transported out of the cell, for example, the protein, whether it be endocrine or exocrine, is secreted by a cell. Secretory pathways have been shown conserved from yeast to mammals, and both conventional and unconventional protein secretion pathways have been demonstrated in plants. Chung et al., “An Overview of Protein Secretion in Plant Cells,” MIMB, 1662:19-32, Sep. 1, 2017. Accordingly, identification of secretory proteins in which one or more polynucleotides may be inserted can be identified for particular cells and applications. In embodiments, one of skill in the art can identify secretory proteins based on the presence of a signal peptide, which consists of a short hydrophobic N-terminal sequence.

In embodiments, the protein is secreted by the secretory pathway. In embodiments, the proteins are exocrine secretion proteins or peptides, comprising enzymes in the digestive tract. In embodiments the protein is endocrine secretion protein or peptide, for example, insulin and other hormones released into the blood stream. In other embodiments, the protein is involved in signaling between or within cells via secreted signaling molecules, for example, paracrine, autocrine, endocrine or neuroendocrine. In embodiments, the secretory protein is selected from the group of cytokines, kinases, hormones and growth factors that bind to receptors on the surface of target cells.

As described, secretory proteins include hormones, enzymes, toxins, and antimicrobial peptides. Examples of secretory proteins include serine proteases (e.g., pepsins, trypsin, chymotrypsin, elastase and plasminogen activators), amylases, lipases, nucleases (e.g. deoxyribonucleases and ribonucleases), peptidases enzyme inhibitors such as serpins (e.g., al-antitrypsin and plasminogen activator inhibitors), cell attachment proteins such as collagen, fibronectin and laminin, hormones and growth factors such as insulin, growth hormone, prolactin platelet-derived growth factor, epidermal growth factor, fibroblast growth factors, interleukins, interferons, apolipoproteins, and carrier proteins such as transferrin and albumins. In some examples, the secretory protein is insulin or a fragment thereof. In one example, the secretory protein is a precursor of insulin or a fragment thereof. In certain examples, the secretory protein is c-peptide. In a preferred embodiment, the one or more polynucleotides is inserted in the middle of the c-peptide. In some aspects, the secretory protein is GLP-1, glucagon, betatrophin, pancreatic amylase, pancreatic lipase, carboxypeptidase, secretin, CCK, a PPAR (e.g. PPAR-alpha, PPAR-gamma, PPAR-delta or a precursor thereof (e.g. preprotein or preproprotein). In aspects, the secretory protein is fibronectin, a clotting factor protein (e.g. Factor VII, VIII, IX, etc.), α2-macroglobulin, al-antitrypsin, antithrombin III, protein S, protein C, plasminogen, α2-antiplasmin, complement components (e.g. complement component C1-9), albumin, ceruloplasmin, transcortin, haptoglobin, hemopexin, IGF binding protein, retinol binding protein, transferrin, vitamin-D binding protein, transthyretin, IGF-1, thrombopoietin, hepcidin, angiotensinogen, or a precursor protein thereof. In aspects, the secretory protein is pepsinogen, gastric lipase, sucrase, gastrin, lactase, maltase, peptidase, or a precursor thereof. In aspects, the secretory protein is renin, erythropoietin, angiotensin, adrenocorticotropic hormone (ACM), amylin, atrial natriuretic peptide (ANP), calcitonin, ghrelin, growth hormone (GH), leptin, melanocyte-stimulating hormone (MSH), oxytocin, prolactin, follicle-stimulating hormone (FSH), thyroid stimulating hormone (TSH), thyrotropin-releasing hormone (TRH), vasopressin, vasoactive intestinal peptide, or a precursor thereof.

### Immunomodulatory Polypeptides

In certain example embodiments, the one or more polypeptides may comprise one or more immunomodulatory protein. In certain embodiments, the present invention provides for modulating immune states. The immune state can be modulated by modulating T cell function or dysfunction. In particular embodiments, the immune state is modulated by expression and secretion of IL-10 and/or other cytokines as described elsewhere herein. In certain embodiments, T cells can affect the overall immune state, such as other immune cells in proximity.

The polynucleotides may encode one or more immunomodulatory proteins, including immunosuppressive proteins. The term “immunosuppressive” means that immune response in an organism is reduced or depressed. An immunosuppressive protein may suppress, reduce, or mask the immune system or degree of response of the subject being treated. For example, an immunosuppressive protein may suppress cytokine production, downregulate or suppress self-antigen expression, or mask the MHC antigens. As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”) and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some cases, the immunosuppressive proteins may exert pleiotropic functions. In some cases, the immunomodulatory proteins may maintain proper regulatory T cells versus effector T cells (Treg/Teff) balance. For examples, the immunomodulatory proteins may expand and/or activate the Tregs and blocks the actions of Teffs, thus providing immunoregulation without global immunosuppression. Target genes associated with immune suppression include, for example, checkpoint inhibitors such PD1, Tim3, Lag3, TIGIT, CTLA-4, and combinations thereof.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, effector Th, memory Th, regulatory Th, CD4+/CD8+ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naïve B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (h) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 300%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely over time (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated CD8+ cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

The invention provides compositions and methods for modulating T cell balance. The invention provides T cell modulating agents that modulate T cell balance. For example, in some embodiments, the invention provides T cell modulating agents and methods of using these T cell modulating agents to regulate, influence or otherwise impact the level of and/or balance between T cell types, e.g., between Th17 and other T cell types, for example, Th1-like cells. For example, in some embodiments, the invention provides T cell modulating agents and methods of using these T cell modulating agents to regulate, influence or otherwise impact the level of and/or balance between Th17 activity and inflammatory potential. As used herein, terms such as “h17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “M2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

In some examples, immunomodulatory proteins may be immunosuppressive cytokines. In general, cytokines are small proteins and include interleukins, lymphokines and cell signal molecules, such as tumor necrosis factor and the interferons, which regulate inflammation, hematopoiesis, and response to infections. Examples of immunosuppressive cytokines include interleukin 10 (IL-10), TGF-β, IL-Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-λ, bFGF, CCL2, CXCL1, CXCL8, CXCL12, CX3CL1, CXCR4, TNF-α and VEGF. Examples of immunosuppressive proteins may further include FOXP3, AHR, TRP53, IKZF3, IRF4, IRF1, and SMAD3. In one example, the immunosuppressive protein is IL-10. In one example, the immunosuppressive protein is IL-6. In one example, the immunosuppressive protein is IL-2.

### Anti-Fibrotic Proteins

In certain example embodiments, the one or more polypeptides may comprise an anti-fibrotic protein. Examples of anti-fibrotic proteins include any protein that reduces or inhibits the production of extracellular matrix components, fibronectin, proteoglycan, collagen, elastin, TGIFs, and SMAD7. In embodiments, the anti-fibrotic protein is a peroxisome proliferator-activated receptor (PPAR), or may include one or more PPARs. In some embodiments, the protein is PPARα, PPAR γ is a dual PPARα/γ. Derosa et al., “The role of various peroxisome proliferator-activated receptors and their ligands in clinical practice” Jan. 18, 2017 J. Cell. Phys. 223:1 153-161.

Proteins that Promote Tissue Regeneration and/or Transplant Survival Functions

In certain example embodiments, the one or more polypeptides may comprise proteins that promote tissue regeneration and/or transplant survival functions. In some cases, such proteins may induce and/or up-regulate the expression of genes for pancreatic β cell regeneration. In some cases, the proteins that promote transplant survival and functions include the products of genes for pancreatic β cell regeneration. Such genes may include proislet peptides that are proteins or peptides derived from such proteins that stimulate islet cell neogenesis. Examples of genes for pancreatic β cell regeneration include Reg1, Reg2, Reg3, Reg4, human proislet peptide, parathyroid hormone-related peptide (1-36), glucagon-like peptide-1 (GLP-1), extendin-4, prolactin, Hgf, Igf-1, Gip-1, adipsin, resistin, leptin, IL-6, IL-10, Pdx1, Ptfa1, Mafa, Pax6, Pax4, Nkx6.1, Nkx2.2, PDGF, vglycin, placental lactogens (somatomammotropins, e.g., CSH1, CHS2), isoforms thereof, homologs thereof, and orthologs thereof. In certain embodiments, the protein promoting pancreatic B cell regeneration is a cytokine, myokine, and/or adipokine.

### Hormones

In certain embodiments, the one or more polynucleotides may comprise one or more hormones. The term “hormone” refers to polypeptide hormones, which are generally secreted by glandular organs with ducts. Hormones include proteins from natural sources or from recombinant cell culture and biologically active equivalents of the native sequence hormone, including synthetically produced small-molecule entities and pharmaceutically acceptable derivatives and salts thereof. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, inhibin; activin; mullerian-inhibiting substance; and thrombopoietin, growth hormone (GH), adrenocorticotropic hormone (ACTH), dehydroepiandrosterone (DHEA), cortisol, epinephrine, thyroid hormone, estrogen, progesterone, placental lactogens (somatomammotropins, e.g. CSH1, CHS2), testosterone. and neuroendocrine hormones. In certain examples, the hormone is secreted from pancreas, e.g., insulin, glucagon, somatostatin, pancreatic polypeptide and ghrelin. In some examples, the hormone is insulin.

Hormones herein may also include growth factors, e.g., fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, and glucocorticoids. In a particular embodiment, the hormone is insulin or incretins such as exenatide, GLP-1.

### Neurohormones

In embodiments, the secreted peptide is a neurohormone, a hormone produced and released by neuroendocrine cells. Example neurohormones include Thyrotropin-releasing hormone, Corticotropin-releasing hormone, Histamine, Growth hormone-releasing hormone, Somatostatin, Gonadotropin-releasing hormone, Serotonin, Dopamine, Neurotensin, Oxytocin, Vasopressin, Epinephrine, and Norepinephrine.

### Anti-Microbial Proteins

In some embodiments, the one or more polypeptides may comprise one or more anti-microbial proteins. In embodiments where the cell is mammalian cell, human host defense antimicrobial peptides and proteins (AMPs) play a critical role in warding off invading microbial pathogens. In certain embodiments, the anti-microbial is a-defensin HD-6, HNP-1 and β-defensin hBD-3, lysozyme, cathelcidin LL-37, C-type lectin RegIIIalpha, for example. See, e.g., Wang, “Human Antimicrobial Peptide and Proteins” Pharma, May 2014, 7(5): 545-594, incorporated herein by reference.

### Anti-Fibrillating Proteins

In certain example embodiments, the one or more polypeptides may comprise one or more anti-fibrillating polypeptides. The anti-fibrillating polypeptide can be the secreted polypeptide. In some embodiments, the anti-fibrillating polypeptide is co-expressed with one or more other polynucleotides and/or polypeptides described elsewhere herein. The anti-fibrillating agent can be secreted and act to inhibit the fibrillation and/or aggregation of endogenous proteins and/or exogenous proteins that it may be co-expressed therewith. In some embodiments, the anti-fibrillating agent is P4 (VITYF (SEQ ID NO: 55)), P5 (VVVVV (SEQ ID NO: 56)), KR7 (KPWWPRR (SEQ ID NO: 57)), NK9 (NIVNVSLVK (SEQ ID NO: 58)), iAb5p (Leu-Pro-Phe-Phe-Asp (SEQ ID NO: 59)), KLVF (SEQ ID NO: 60) and derivatives thereof, indolicidin, carnosine, a hexapeptide as set forth in Wang et al. 2014. ACS Chem Neurosci. 5:972-981, alpha sheet peptides having alternating D-amino acids and L-amino acids as set forth in Hopping et al. 2014. Elife 3:e01681, D-(PGKLVYA (SEQ ID NO: 61)), RI-OR2-TAT, cyclo(17, 21)-(Lys17, Asp21)A_(1-28), SEN304, SEN1576, D3, R8-Aβ(25-35), human yD-crystallin (HGD), poly-lysine, heparin, poly-Asp, polyGl, poly-L-lysine, poly-L-glutamic acid, LVEALYL (SEQ ID NO: 62), RGFFYT (SEQ ID NO: 63), a peptide set forth or as designed/generated by the method set forth in U.S. Pat. No. 8,754,034, and combinations thereof. In aspects, the anti-fibrillating agent is a D-peptide. In aspects, the anti-fibrillating agent is an L-peptide. In aspects, the anti-fibrillating agent is a retro-inverso modified peptide. Retro-inverso modified peptides are derived from peptides by substituting the L-amino acids for their D-counterparts and reversing the sequence to mimic the original peptide since they retain the same spatial positioning of the side chains and 3D structure. In aspects, the retro-inverso modified peptide is derived from a natural or synthetic Aβ peptide. In some embodiments, the polynucleotide encodes a fibrillation resistant protein. In some embodiments, the fibrillation resistant protein is a modified insulin, see e.g., U.S. Pat. No. 8,343,914.

### Antibodies

In certain embodiments, the one or more polypeptides may comprise one or more antibodies. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

### Protease Cleavage Sites

The one or more cargo polypeptides, as exemplified above, may comprise one or more protease cleavage sites, i.e., amino acid sequences that can be recognized and cleaved by a protease. The protease cleavage sites may be used for generating desired gene products (e.g., intact gene products without any tags or portion of other proteins). The protease cleavage site may be one end or both ends of the protein. Examples of protease cleavage sites that can be used herein include an enterokinase cleavage site, a thrombin cleavage site, a Factor Xa cleavage site, a human rhinovirus 3C protease cleavage site, a tobacco etch virus (TEV) protease cleavage site, a dipeptidyl aminopeptidase cleavage site and a small ubiquitin-like modifier (SUMO)/ubiquitin-like protein-1 (ULP-1) protease cleavage site. In certain examples, the protease cleavage site comprises Lys-Arg.

### Small Molecules

In some embodiments, the cargo molecule is a small molecule. Techniques and methods of coupling peptides to small molecule agents are generally known in the art and can be applied here to couple a targeting moiety effective to target a CNS cell to a small molecule cargo. Small molecules include, without limitation, hormones, immunomodulators, antipyretics, anxiolytics, antipsychotics, analgesics, antispasmodics, anti-inflammatories, anti-histamines, anti-infectives, radiation sensitizers, chemotherapeutics.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosteron Cortisol). Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-α, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g., ibuprofen, naproxen, ketoprofen, and nimesulide), aspirin and related salicylates (e.g., choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Suitable anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, abendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscamet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telvancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g., lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicyclic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

### Engineered Viral Capsids and Encoding Polynucleotides

Described herein are exemplary embodiments of engineered viral polypeptides, (e.g., capsid polypeptides), such as adeno-associated virus (AAV) viral polypeptides (e.g., capsid polypeptides), that can be engineered to confer cell-specific tropism to an engineered viral particle (AAV particle) that contains the engineered viral polypeptide (s). The engineered viral polypeptide (s) (e.g., capsid(s)) can be included in an engineered virus particle, and can confer cell-specific tropism, such as CNS-specific tropism, reduced immunogenicity, or both to the engineered viral (e.g., an AAV) particle. As is described elsewhere herein, the particles can include a cargo. In this way, the particles can be a cell-specific delivery vehicle for a cargo. The engineered viral capsids described herein can include one or more engineered viral capsid polypeptides described herein. Engineered viral capsid polypeptides can be lentiviral, retroviral, adenoviral, or AAV. Engineered capsids can contain one or more of the viral capsid polypeptides. Engineered virus particles can include one or more of the engineered viral capsid polypeptides and thus contain an engineered viral capsid. The engineered viral capsid polypeptides, capsids, and/or viral particles that contain one or more CNS-specific targeting moieties containing or composed of one or more n-mer inserts described elsewhere herein. In some embodiments, the engineered viral capsid polypeptides, viral capsids, and/or viral particles can have a CNS-specific tropism conferred to it by the one or more n-mer inserts contained therein.

The CNS-specific n-mer inserts and targeting moieties can be encoded in whole or in part by a polynucleotide. The engineered viral capsid and/or viral capsid polypeptides can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

The engineered AAV capsids can be variants of wild-type AAV capsids. In some embodiments, the wild-type AAV capsids can be composed of VP1, VP2, VP3 capsid polypeptides or a combination thereof. In other words, the engineered AAV capsids can include one or more variants of a wild-type VP1, wild-type VP2, and/or wild-type VP3 capsid polypeptides. In some embodiments, the serotype of the reference wild-type AAV capsid can be AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 or any combination thereof. In some embodiments, the serotype of the wild-type AAV capsid can be AAV-9. The engineered AAV capsids can have a different tropism than that of the reference wild-type AAV capsid.

The engineered AAV capsid can contain 1-60 engineered capsid polypeptides. In some embodiments, the engineered AAV capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid polypeptides. In some embodiments, the engineered AAV capsid can contain 0-59 wild-type AAV capsid polypeptides. In some embodiments, the engineered AAV capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type AAV capsid polypeptides.

In some embodiments, the engineered AAV capsid polypeptide can have an n-mer amino acid insert (also referred herein as an “n-mer insert”), where n can be at least 3 amino acids. In some embodiments, n can be 3, 4, 5, 6, 7, 8, 9, 10, 11, 12, 13, 14, or 15 amino acids. In some embodiments, the engineered AAV capsid can have a 6-mer or 7-mer amino acid insert. In some embodiments, the n-mer amino acid inset can be inserted between two amino acids in the wild-type viral polypeptide (VP) (or capsid polypeptide). In some embodiments, the n-mer insert can be inserted between two amino acids in a variable amino acid region in an AAV capsid polypeptide. The core of each wild-type AAV viral polypeptide contains an eight-stranded beta-barrel motif (betaB to betaI) and an alpha-helix (alphaA) that are conserved in autonomous parvovirus capsids (see e.g., DiMattia et al. 2012. J. Virol. 86(12):6947-6958). Structural variable regions (VRs) occur in the surface loops that connect the beta-strands, which cluster to produce local variations in the capsid surface. AAVs have 12 variable regions (also referred to as hypervariable regions) (see e.g., Weitzman and Linden. 2011. “Adeno-Associated Virus Biology.” In Snyder, R. O., Moullier, P. (eds.) Totowa, NJ: Humana Press). In some embodiments, one or more n-mer inserts can be inserted between two amino acids in one or more of the 12 variable regions in the wild-type AVV capsid polypeptides. In some embodiments, the one or more n-mer inserts can be each be inserted between two amino acids in VR-I, VR-II, VR-III, VR-IV, VR-V, VR-VI, VR-VII, VR-III, VR-IX, VR-X, VR-XI, VR-XII, or a combination thereof. In some embodiments, the n-mer can be inserted between two amino acids in the VR-III of a capsid polypeptide. In some embodiments, the engineered capsid can have an n-mer inserted between any two contiguous amino acids between amino acids 262 and 269, between any two contiguous amino acids between amino acids 327 and 332, between any two contiguous amino acids between amino acids 382 and 386, between any two contiguous amino acids between amino acids 452 and 460, between any two contiguous amino acids between amino acids 488 and 505, between any two contiguous amino acids between amino acids 545 and 558, between any two contiguous amino acids between amino acids 581 and 593, between any two contiguous amino acids between amino acids 704 and 714 of an AAV9 viral polypeptide. In some embodiments, the engineered capsid can have an n-mer inserted between amino acids 588 and 589 of an AAV9 viral polypeptide. In some embodiments, the engineered capsid can have an n-mer insert inserted between amino acids 588 and 589 of an AAV9 viral polypeptide. In some embodiments, the engineered capsid can have an n-mer insert inserted between amino acids 598-599 of an AAV9 viral polypeptide SEQ ID NO: 1 is a reference AAV9 capsid sequence for at least referencing the insertion sites discussed above. It will be appreciated that n-mers can be inserted in analogous positions in AAV viral polypeptides of other serotypes. In some embodiments as previously discussed, the n-mer(s) can be inserted between any two contiguous amino acids within the AAV viral polypeptide and in some embodiments the insertion is made in a variable region.

In certain example embodiments, the targeting moiety comprises a viral polypeptide.

In certain example embodiments, the viral polypeptide is a capsid polypeptide.

In certain example embodiments, wherein the n-mer insert(s) is/are incorporated into the viral polypeptide such that the n-mer insert, or at least the P motif, or at least the double valine motifs located between two amino acids of the viral polypeptide such that the n-mer insert, or at least the P motif, or at least the double valine motif is external to a viral capsid.

In certain example embodiments, the viral polypeptide is an adeno associated virus (AAV) polypeptide.

In certain example embodiments, the AAV polypeptide is an AAV capsid polypeptide.

In certain example embodiments, one or more of the n-mer insert(s) are each incorporated into the AAV polypeptide such that n-mer motif, or at least the P motif, or at least the double valine motif is inserted between any two contiguous amino acids independently selected from amino acids 262-269, 327-332, 382-386, 452-460, 488-505, 527-539, 545-558, 581-593, 598-599, 704-714, or any combination thereof in an AAV9 capsid polypeptide or in an analogous position in an AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, at least one of the n-mer inserts is incorporated into the AAV polypeptide such that n-mer insert(s), or at least the P motif(s), or at least the double valine motif(s) is inserted between amino acids 588 and 589 in an AAV9 capsid polypeptide or in an analogous position in an AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, at least one of the n-mer insert(s) is incorporated into the AAV polypeptide such that the n-mer insert(s), or at least the P motif, or at least the double valine motif is inserted between amino acids 598-599 in an AAV9 capsid polypeptide or in an analogous position in an AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide

SEQ ID NO: 1 AAV9 capsid (wild-type) reference
Sequence:
MAADGYLPDWLEDNLSEGIREWWALKPGAPQPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVE
QSPQEPDSSAGIGKSGAQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISNSTSGGSSNDNAYFGYSTPWGYFDFNRFHCHFS
PRDWQRLINNNWGFRPKRLNFKLFNIQVKEVTDNNGVKTIANNLTSTVQ
VFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLID
QYLYYLSKTINGSGQNQQTLKFSVAGPSNMAVQGRNYIPGPSYRQQRVS
TTVTQNNNSEFAWPGASSWALNGRNSLMNPGPAMASHKEGEDRFFPLSG
SLIFGKQGTGRDNVDADKVMITNEEEIKTTNPVATESYGQVATNHQSAQ
AQAQTGWVQNQGILPGMVWQDRDVYLQGPIWAKIPHTDGNFHPSPLMGG
FGMKHPPPQILIKNTPVPADPPTAFNKDKLNSFITQYSTGQVSVEIEWE
LQKENSKRWNPEIQYTSNYYKSNNVEFAVNTEGVYSEPRPIGTRYLTRN
L

In some embodiments, an AAV capsid and/or AAV vector can contain one or more targeting moieties having one or more n-mer inserts containing one or more P-motifs. n-mer inserts containing or being P-motifs are described in greater detail elsewhere herein. In some embodiments, an AAV capsid and/or AAV vector can contain one or more targeting moieties having one or more n-mer inserts that are each immediately preceded by AQ or DG in the AAV capsid and/or vector in which they are inserted. In other words, the n-mer insert can be inserted into an AAV capsid and/or AAV vector between two contiguous amino acids such that the two residues preceding the n-mer insert are AQ or DG. In some embodiments, the n-mer insert is engineered such that the two C-terminal residues of the n-mer insert and/or preceding a P-motif of an n-mer insert are AQ or DG. In some embodiments, amino acids 587 and 588 of the AAV capsid or vector or analogous amino acids thereto are DG or DG.

In some embodiments, an AAV capsid (such as a CNS-specific AAV capsid) contains an n-mer insert that is or contains an n-mer motif, a P-motif, and/or a double valine motif such as any one or more as set forth in Tables 1-38, S1, or FIGS. 15A, 15B, 16A, 16B, 16C, 19A-19C. In some embodiments, insertion of the n-mer insert in an AAV capsid can result in cell, tissue, organ, specific engineered AAV capsids. In some embodiments, the engineered viral polypeptide, engineered viral capsid polypeptide, engineered viral capsid, and/or engineered viral particle has specificity for one or more types of CNS cells and/or tissue. In some embodiments, an engineered viral polypeptide, engineered viral capsid polypeptide, engineered viral capsid and/or engineered viral particle having an n-mer insert that is or contains a P-motif (e.g., those described in Tables 8 and S1 or FIGS. 15A, 15B, 16A, 16B, 16C, 19A-19C and elsewhere herein), has specificity for one or more types of CNS cells and/or tissue.

In some embodiments, the n-mer insert(s) in an AAV capsid is or includes a “P motif” and/or double valine motif. N-mer inserts, P motifs and double valine motifs are described in greater detail elsewhere herein. In some embodiments, an AAV capsid includes an n-mer insert comprising or consisting of a P-motif having the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7. In some embodiments, the an AAV capsid includes an n-mer insert comprising or consisting of a P-motif having the amino acid sequence XₘPX₁QGTX₃RXₙ (SEQ ID NO: 8581), where X₁, X₃, Xₙ, are each selected from any amino acid, where m is 0, 1, 2, or 3, and where n is 0, 1, 2, 3, 4, 5, 6, or 7. In some embodiments, an AAV capsid includes an n-mer insert comprising or consisting of a P-motif having the amino acid sequence PX₁QGTX₃RXₙ (SEQ ID NO: 2), where X₁, X₃, Xₙ, are each selected from any amino acid and where n is 0, 1, 2, 3, 4, 5, 6, or 7. In certain example embodiments, X₂ of the P motif is Q, P, E, or H. In certain example embodiments, X₁ of the P motif is a polar amino acid, optionally a polar uncharged amino acid. In certain example embodiments, X₃ of the P motif is a nonpolar amino acid. In certain example embodiments, X₁ of the double valine motif is R, K, V, or W. In certain example embodiments, X₂ of the double valine motif is T, S, V, Y or R.

In some embodiments, the AAV capsid includes an n-mer insert that is or includes a double valine motif having the amino acid sequence of the amino acid sequence XₘX₁X₂VX₃X₄VX₅Xₙ, wherein X₁, X₂, X₃, Xₘ, and Xₙ, are each independently selected from any amino acid, wherein m is 0, 1, 2, or 3, and wherein n is 0, 1, 2, 3, 4, 5, 6, or 7. Double valine motifs are further described in greater detail elsewhere herein. In certain example embodiments, X₃ of the double valine motif is G, P, or S. In certain example embodiments, X₄ of the double valine motif is S, D, or T. In certain example embodiments, X₅ of the double valine motif is Y, G, S, or L.

Exemplary, non-limiting n-mer inserts, P motifs, and double valine motifs are shown at least in e.g., Table 1-38, S1 and FIGS. 15A, 15B, 16A, 16B, 16C, 19A-19C. N-mer inserts, P-motifs, and double valine motifs are further described elsewhere herein.

In some embodiments, one or more n-mer inserts can be as set forth in any one or more of Tables 1, 2, 3, 8, S1 and FIG. 15A, 15B, 16A, 16B, 16C, 19A, 19B, or 19C can be included in a CNS specific engineered capsid.

As is described above and demonstrated in e.g., Table 1 and the Working Examples, the n-mer insert can be inserted into an AAV vector between two contiguous amino acids where the amino acids in the AAV vector immediately preceding the n-mer insert can be DG or AQ. In connection with Table 1, the first two amino acids shown in the variants are either AQ or DG, which denote amino acid residues (e.g., residues 587 and 588 that were either endogenous to the vector or show amino acid residues that were part of the n-mer insert that replaced residues at position 587 and 588 in the AAV vector to which the n-mer insert was introduced. Each n-mer insert of Table 1 was tested in both configurations (e.g., with AQ and DG as amino acids 587 and 588 of the AAV).

In some embodiments, the n-mer insert (such as a 7-mer insert) can be inserted into an AAV vector between two contiguous amino acids where the amino acids in the AAV vector immediately preceding the n-mer insert can be DG or AQ. In some embodiments, the DG or AQ are the amino acids immediately preceding the n-mer insert in the capsid polypeptide when the n-mer insert is included in a capsid polypeptide, particularly an AAV capsid polypeptide. Without being bound by theory, inserts including a DG or AQ at the C terminal end or are inserted into a capsid polypeptide, such as an AAV capsid polypeptide, such that the insert(s) are immediately following an AQ or DG of the capsid polypeptide, may be able to transduce more hosts, such as more strains or species. In some embodiments, amino acids 587 and 588 of the AAV or analogous amino acids thereto are DG. In some embodiments, amino acids 587 and 588 of the AAV or analogous amino acids thereto are AQ. In some embodiments, amino acids 587 and 588 of the AAV or analogous amino acids thereto are AQ and are followed by an n-mer insert. In some embodiments, amino acids 587 and 588 of the AAV or analogous amino acids thereto are DG and are followed by an n-mer insert.

In some embodiments, the n-mer insert is such that when included in a host polypeptide (e.g., viral or AAV polypeptide, such as a capsid polypeptide) one or more residues of the host polypeptide are replaced with one or more of that from the n-mer insert. In some embodiments, when a C terminal AQ or DG are included in the n-mer insert but are not part of a P motif, the AQ or DG can optionally replace 1 or 2 amino acid residues immediately preceding where the P motif or double valine motif is to be inserted. For example, in some embodiments, where the P motif is desired to be inserted between e.g., 588 and 589 in an AAV9 or position analogous thereto in other AAVs, the n-mer insert can contain e.g., [e.g., AQ or DG]-[P motif or double valine motif]-Xn, where Xn is as described elsewhere herein with respect to the P motifs, where AQ or DG replaces residues 587 and 588 of the AAV9 or position analogous thereto in other AAVs leaving the P motif or double valine motif to be effectively inserted between positions 588 and 589 of the AAV9 or position analogous thereto in other AAVs. It will be appreciated that such an approach can be extrapolated to other host polypeptides besides AAVs as well as other positions within AAVs. Further this can be extrapolated to other C-terminal amino acids besides AQ or DG as the case may be (e.g., Xₘ in the context of P motifs or double valine motifs).

In some embodiments, the n-mer insert confers CNS transduction efficiency to the targeting moiety. At least Tables 1-3, 7-8, S1, FIGS. 15A, 15B, 16A, 16B, 16C, 19A-19C represent exemplary variants having CNS transduction efficiency. As further discussed in the Working Examples herein, engineered AAV variants such as at least in Table 1 were able to transduce cells from multiple strains of mice. This is in contrast to other AAVs, which at least in some cases, can only transduce certain strains of mice.

In some embodiments, an AAV capsid can contain one or more targeting moieties having one or more n-mer inserts that are each immediately preceded by AQ and wherein the n-mer insert is KTVGTVY (SEQ ID NO: 3), RSVGSVY (SEQ ID NO: 4), RYLGDAS (SEQ ID NO: 5), WVLPSGG (SEQ ID NO: 6), VTVGSIY (SEQ ID NO: 7), VRGSSIL (SEQ ID NO: 8), RHHGDAA (SEQ ID NO: 9), VIQAMKL (SEQ ID NO: 10), LTYGMAQ (SEQ ID NO: 11), LRIGLSQ (SEQ ID NO: 12), GDYSMIV (SEQ ID NO: 13), VNYSVAL (SEQ ID NO: 14), RHIADAS (SEQ ID NO: 15), RYLGDAT (SEQ ID NO: 16), QRVGFAQ (SEQ ID NO: 17), QIAHGYST (SEQ ID NO: 18), WTLESGH (SEQ ID NO: 19); or GENSARW (SEQ ID NO: 20). In some embodiments, an AAV capsid can contain one or more targeting moieties having one or more n-mer inserts that are each immediately preceded by DG and wherein the n-mer insert is REQQKLW (SEQ ID NO: 21), ASNPGRW (SEQ ID NO: 22), WTLESGH (SEQ ID NO: 23), REQKKLW (SEQ ID NO: 24), ERLLVQL (SEQ ID NO: 25); or RMQRTLY (SEQ ID NO: 26). In some embodiments, amino acids 587 and 588 of the AAV or analogous amino acids thereto are DG and are followed by a 7-mer amino acid insert. In some embodiments, amino acids 587 and 588 of the AAV or analogous amino acids thereto are DG and are followed by a 7-mer amino acid insert, where the 7-mer insert is REQQKLY (SEQ ID NO: 64), ASNPGRW (SEQ ID NO: 22), WTLESGH (SEQ ID NO: 23, REQKKLW (SEQ ID NO: 24), ERLLVQL (SEQ ID NO: 25); or RMQRTLY (SEQ ID NO: 26).

In some embodiments, the AAV capsids can be CNS-specific. In some embodiments, CNS-specificity of the engineered AAV capsid is conferred by a CNS specific n-mer insert incorporated in the engineered AAV capsid. While not intending to be bound by theory, it is believed that the n-mer insert confers a 3D structure to or within a domain or region of the engineered AAV capsid such that the interaction of an engineered AAV containing said engineered AAV capsid has increased or improved interactions (e.g., increased affinity) with a cell surface receptor and/or other molecule on the surface of an endothelial and/or a CNS cell. In some embodiments the cell surface receptor is AAV receptor (AAVR). In some embodiments, the cell surface receptor is a CNS cell specific AAV receptor. In some embodiments, a CNS specific engineered AAV containing the CNS-specific capsid can have an increased transduction rate, efficiency, amount, or a combination thereof in a CNS cell as compared to other cell types and/or other AAVs that do not contain a CNS-specific engineered AAV capsid.

TABLE 1
Exemplary CNS n-mer inserts

| Variant | | |
|---|---|---|
| Initial ″AQ″ or ″DG″ in the | | |
| inserts in Table 1 correspond | | |
| to the two amino acids in the | | |
| targeting moiety that immediately | | |
| precede the ″n-mer insert″ in a | | |
| targeting moiety or composition | | |
| (e.g., AA 587 and 588 of an AAV9 | | |
| that has an n-mer insert placed | CNS Transduction efficiency | |
| between AA 588 and 589). | Score | SEQ ID NO: |
| AQRSVGSVY | 46000 | 65 |
| AQKTVGTVY | 45980 | 66 |
| AQRYLGDAS | 40592 | 67 |
| DGREQQKLW | 39151 | 68 |
| AQWVLPSGG | 37597 | 69 |
| AQVTVGSIY | 32968 | 70 |
| AQVRGSSIL | 32330 | 71 |
| AQRHHGDAA | 32171 | 72 |
| AQVIQAMKL | 32127 | 73 |
| AQLTYGMAQ | 31956 | 74 |
| AQLRIGLSQ | 31710 | 75 |
| AQGDYSMIV | 31497 | 76 |
| AQVNYSVAL | 31271 | 77 |
| AQRYSGDAS | 31198 | 78 |
| AQRYSGDSV | 29860 | 79 |
| AQRHIADAS | 29554 | 80 |
| AQRYLGDAT | 29527 | 81 |
| AQQRVGFAQ | 29454 | 82 |
| AQIAHGYST | 28216 | 83 |
| AQWTLESGH | 27471 | 84 |
| AQGENSARW | 27287 | 85 |
| DGASNPGRW | 24583 | 86 |
| AQLAVGQKW | 24445 | 87 |
| AQVKLGYSQ | 23912 | 88 |
| AQEAGSARW | 23888 | 89 |
| AQLNYSVSL | 21972 | 90 |
| AQWAISDGY | 21970 | 91 |
| AQRGPGLSQ | 21738 | 92 |
| DGWTLESGH | 20534 | 93 |
| AQRYVGESS | 19635 | 94 |
| DGREQKKLW | 17695 | 95 |
| AQFTLTTPK | 15607 | 96 |
| DGERLLVQL | 15513 | 97 |
| AQEDLLRLR | 14920 | 98 |
| AQPIIEHAV | 12837 | 99 |
| DGRMQRTLY | 12453 | 100 |
| DGWAISDGY | 10828 | 101 |
| AQRYISDSA | 10788 | 102 |
| AQWSTSSGF | 10614 | 103 |
| AQWSLGSGH | 10498 | 104 |
| AQWSQSSGY | 10258 | 105 |
| DGVRGSSIL | 9714 | 106 |
| AQIMLGYST | 9404 | 107 |
| DGKLADSVP | 9356 | 108 |
| AQASNPGRW | 9173 | 109 |
| AQHVENWHI | 8680 | 110 |
| AQVAGSSIL | 8645 | 111 |
| DGRQQQKLW | 8393 | 112 |
| DGTVNNDRF | 8028 | 113 |
| DGMSANERT | 8000 | 114 |
| AQATVAGQF | 7885 | 115 |
| DGRDQQKLW | 7761 | 116 |
| AQGKSPGVW | 7685 | 117 |
| DGGASNGGT | 7674 | 118 |
| AQSLVTSST | 6782 | 119 |
| AQLLYGYSS | 6779 | 120 |
| DGVTELTKF | 6655 | 121 |
| AQALVQNGV | 6638 | 122 |
| AQVLESNPR | 6572 | 123 |
| AQPASHEVL | 6460 | 124 |
| AQAGVQNAL | 6452 | 125 |
| DGKEISVSV | 6420 | 126 |
| AQGLNERVA | 6410 | 127 |
| DGQVAQQGA | 6393 | 128 |
| DGGVAGTNT | 6386 | 129 |
| DGASAQGAL | 6382 | 130 |
| AQAGVSSQT | 6357 | 131 |
| AQKNRRHSV | 6312 | 132 |
| AQKVDSAQL | 6311 | 133 |
| AQYTLSQGW | 6310 | 134 |
| DGQSVDRSK | 6293 | 135 |
| AQASASSPR | 6266 | 136 |
| DGRYVGESS | 6252 | 137 |
| DGLGHNAGV | 6239 | 138 |
| AQPNERINV | 6142 | 139 |
| AQVMSGTSH | 6122 | 140 |
| DGVLVSPGP | 6000 | 141 |
| DGVGISSGV | 6000 | 142 |
| DGSGETLRI | 5977 | 143 |
| DGSTEGAAL | 5954 | 144 |
| AQTSLSQDR | 5943 | 145 |
| AQSANPVVT | 5937 | 146 |
| DGVLASNGP | 5898 | 147 |
| AQAHLDNAP | 5893 | 148 |
| DGVVQVTGR | 5875 | 149 |
| DGFAVRLSS | 5855 | 150 |
| DGLVRDTKT | 5811 | 151 |
| DGSGESLSR | 5804 | 152 |
| AQTNEQAQR | 5796 | 153 |
| DGTLANSQR | 5746 | 154 |
| AQLLADKSV | 5680 | 155 |
| DGSQEQRAR | 5679 | 156 |
| AQVNGNTTY | 5655 | 157 |
| AQALAEAGA | 5624 | 158 |
| DGSREGGNV | 5580 | 159 |
| AQMGDSVTI | 5574 | 160 |
| DGLGGSSMG | 5565 | 161 |
| AQGVRDTNI | 5562 | 162 |
| DGSGSTDKL | 5556 | 163 |
| AQASQNSTV | 5493 | 164 |
| AQGGTSSGH | 5462 | 165 |
| AQAADSSVR | 5404 | 166 |
| AQAANSSVR | 5387 | 167 |
| AQWADSKDQ | 5374 | 168 |
| AQPTQGTVR | 5353 | 169 |
| AQGSTDFKT | 5344 | 170 |
| AQVDHGGVV | 5342 | 171 |
| AQGEQQKGW | 5322 | 172 |
| DGIANLAAS | 5311 | 173 |
| DGAGGVRDR | 5299 | 174 |
| DGGSGSGGL | 5252 | 175 |
| DGTLANSER | 5237 | 176 |
| AQKGASVTL | 5236 | 177 |
| AQSNVALTG | 5235 | 178 |
| DGVNYSVAL | 5206 | 179 |
| AQGLNEHGA | 5193 | 180 |
| DGKNPGVYT | 5173 | 181 |
| DGQREAARI | 5173 | 182 |
| AQGLVDSSR | 5168 | 183 |
| DGNGSEGDR | 5157 | 184 |
| DGNVGVVQL | 5144 | 185 |
| AQVTDGVRS | 5109 | 186 |
| AQVIASNEH | 5109 | 187 |
| AQMSVGQSW | 5098 | 188 |
| DGHSLQTSA | 5096 | 189 |
| AQQDGYGTR | 5093 | 190 |
| AQLSNGQGP | 5071 | 191 |
| AQPVTDSKM | 5068 | 192 |
| AQNGTAADR | 5057 | 193 |
| AQIIVDNGS | 5024 | 194 |
| AQEADNHGR | 5023 | 195 |
| AQAADSSGR | 4995 | 196 |
| AQVVDSNNL | 4986 | 197 |
| DGSGANLSY | 4985 | 198 |
| DGKAHDGEV | 4978 | 199 |

TABLE 2
Additional Exemplary CNS n-mer inserts

| | N-mer | SEQ ID | | SEQ ID |
|---|---|---|---|---|
| Rank | insert | NO: | Encoding sequence | NO: |
| 1 | PSQGTLR | 200 | CCTTCTCAGGGGACGCTTCGG | 201 |
| 2 | TDALTTK | 202 | ACTGATGCGCTTACGACTAAG | 203 |
| 3 | PTQGTVR | 204 | CCCACACAAGGCACAGTCCGT | 205 |
| 4 | PTQGTLR | 206 | CCTACTCAGGGGACGCTTCGG | 207 |
| 5 | PTQGTVR | 208 | CCTACTCAGGGGACGGTTCGG | 209 |
| 6 | STIPTMK | 210 | AGTACTATTCCTACTATGAAG | 211 |
| 7 | TDAGDGK | 212 | ACAGACGCGGGGGACGGCAAA | 213 |
| 8 | YQRTESL | 214 | TATCAGAGGACGGAGTCTCTG | 215 |
| 9 | RVDPSGL | 216 | AGAGTCGACCCCAGTGGACTA | 217 |
| 10 | SLVTSST | 218 | TCGCTTGTTACTTCTAGTACG | 219 |
| 11 | LLAGADR | 220 | TTGCTTGCTGGTGCTGATCGT | 221 |
| 12 | STDRESR | 222 | TCCACGGACCGTGAAAGCCGA | 223 |
| 13 | NGYTEGR | 224 | AATGGGTATACGGAGGGGCGT | 225 |
| 14 | PTQGTFR | 226 | CCGACACAAGGAACATTCAGG | 227 |
| 15 | MTGISIV | 228 | ATGACAGGCATCTCTATCGTA | 229 |
| 16 | DGRAELR | 230 | GATGGGCGGGCGGAGTTGCGT | 231 |
| 17 | AADSSAR | 232 | GCCGCTGACTCATCGGCCCGT | 233 |
| 18 | PTQGTIR | 234 | CCTACTCAGGGGACGATTCGG | 235 |
| 19 | LSRGEEK | 236 | CTTTCGAGGGGTGAGGAGAAG | 237 |
| 20 | AIVSIAQ | 238 | GCGATTGTGTCGATTGCTCAG | 239 |
| 21 | LTSGLAA | 240 | TTGACGTCTGGTTTGGCGGCG | 241 |
| 22 | PTQGTFR | 242 | CCTACTCAGGGGACGTTTCGG | 243 |
| 23 | TLAISGR | 244 | ACTTTGGCGATTTCTGGGCGG | 245 |
| 24 | VHSQDVS | 246 | GTCCACAGTCAAGACGTTTCC | 247 |
| 25 | FQVEQVK | 248 | TTTCAGGTTGAGCAGGTTAAG | 249 |
| 26 | NRELALG | 250 | AACCGCGAACTCGCACTCGGG | 251 |
| 27 | SIGDLGK | 252 | AGTATCGGTGACCTAGGTAAA | 253 |
| 28 | TVGHDNK | 254 | ACCGTAGGACACGACAACAAA | 255 |
| 29 | HSKGFDY | 256 | CACAGTAAAGGTTTCGACTAC | 257 |
| 30 | HTQGTLR | 258 | CATACTCAGGGGACGCTTCGG | 259 |
| 31 | PAQGTLR | 260 | CCGGCGCAAGGAACACTACGA | 261 |
| 32 | AGGGDPR | 262 | GCTGGTGGAGGTGACCCCCGA | 263 |
| 33 | LGKADPV | 264 | TTGGGAAAAGCTGACCCAGTA | 265 |
| 34 | ALNEHVA | 266 | GCTCTGAATGAGCATGTGGCG | 267 |
| 35 | GSGGVSV | 268 | GGTTCGGGTGGTGTTAGTGTG | 269 |
| 36 | PSQGTLR | 270 | CCGTCCCAAGGAACACTCAGG | 271 |
| 37 | TGGRDQY | 272 | ACTGGTGGTCGGGATCAGTAT | 273 |
| 38 | YLVTTEN | 274 | TATTTGGTTACTACTGAGAAT | 275 |
| 39 | LSRDVAV | 276 | TTGTCGAGGGATGTGGCGGTT | 277 |
| 40 | RIVDSVP | 278 | AGGATTGTGGATAGTGTTCCG | 279 |
| 41 | KGYDTPM | 280 | AAAGGCTACGACACACCCATG | 281 |
| 42 | TSREEQW | 282 | ACTTCTCGTGAGGAGCAGTGG | 283 |
| 43 | RASADVV | 284 | AGGGCGAGTGCGGATGTTGTG | 285 |
| 44 | NLGAALS | 286 | AACCTTGGGGCTGCCCTATCG | 287 |
| 45 | SVTDIKH | 288 | TCGGTGACGGACATAAAACAC | 289 |
| 46 | FQDTIGV | 290 | TTTCAGGATACGATTGGGGTG | 291 |
| 47 | PNERLAV | 292 | CCTAACGAACGATTGGCAGTC | 293 |
| 48 | HTIAASM | 294 | CACACCATAGCCGCAAGTATG | 295 |
| 49 | NSDLMGR | 296 | AACAGTGACCTAATGGGCCGA | 297 |
| 50 | AGVSASL | 298 | GCGGGTGTTTCTGCGTCGTTG | 299 |

TABLE 3
Exemplary P-motifs

| n-mer | SEQ | | SEQ |
|---|---|---|---|
| insert | ID NO: | Encoding Sequence(s) | ID NO: |
| PSQGTLR | 300 | CCTTCTCAGGGGACGCTTCGG; | 301 |
| | | CCGTCCCAAGGAACACTCAGG | 302 |
| PTQGTVR | 303 | CCCACACAAGGCACAGTCCGT; | 304 |
| | | CCTACTCAGGGGACGGTTCGG | 305 |
| PTQGTLR | 306 | CCTACTCAGGGGACGCTTCGG | 307 |
| PTQGTFR | 308 | CCGACACAAGGAACATTCAGG; | 309 |
| | | CCTACTCAGGGGACGTTTCGG | 310 |
| PTQGTIR | 311 | CCTACTCAGGGGACGATTCGG | 312 |
| PAQGTLR | 313 | CCGGCGCAAGGAACACTACGA | 314 |

Also described herein are polynucleotides that encode the engineered targeting moieties, viral polypeptides (e.g., capsid polypeptides), and other polypeptides described herein, including but not limited to, the engineered AAV capsids described herein. In some embodiments, the engineered AAV capsid encoding polynucleotide can be included in a polynucleotide that is configured to be an AAV genome donor in an AAV vector system that can be used to generate engineered AAV particles described elsewhere herein.

In some embodiments, the AAV capsids or other viral capsids or compositions can be CNS-specific. In some embodiments, CNS-specificity of the engineered AAV or other viral capsid or other composition is conferred by one or more CNS specific n-mer inserts incorporated in the engineered AAV or other viral capsid or other composition described herein. While not intending to be bound by theory, it is believed that the n-mer insert confers a 3D structure to or within a domain or region of the engineered AAV capsid or other viral capsid or other composition such that the interaction of the viral particle or other composition containing the engineered AAV capsid or other viral capsid or other composition described herein has increased or improved interactions (e.g., increased affinity) with a cell surface receptor and/or other molecule on the surface of a CNS cell. In some embodiments, the cell surface receptor is AAV receptor (AAVR). In some embodiments, the cell surface receptor is a CNS cell specific AAV receptor. In some embodiments, the cell surface receptor or other molecule is a cell surface receptor or other molecule selectively expressed on the surface of a CNS cell.

In some embodiments the engineered viral (e.g., AAV) capsid encoding polynucleotide can be operably coupled to a poly adenylation tail. In some embodiments, the poly adenylation tail can be an SV40 poly adenylation tail. In some embodiments, the viral (e.g., AAV) capsid encoding polynucleotide can be operably coupled to a promoter. In some embodiments, the promoter can be a tissue specific promoter. In some embodiments, neurons an/or supporting cells (e.g., astrocytes, glial cells, Schwann cells, etc.), and combinations thereof. In some embodiments, the promoter can be a constitutive promoter. Suitable tissue specific promoters and constitutive promoters are discussed elsewhere herein and are generally known in the art and can be commercially available.

Suitable neuronal tissue/cell specific promoters include, but are not limited to, GFAP promoter (astrocytes), SYN1 promoter (neurons), and NSE/RU5′ (mature neurons).

Other suitable CNS specific promoters can include, but are not limited to, neuroactive peptide cholecystokinin (CCK) (see e.g., Chhatawl et al. Gene Therapy volume 14, pages 575-583(2007)), a brain specific DNA MiniPromoter (such as any of those identified for brain or pan-neronal expression as in de Leeuw et al. Mol. Therapy. 1(5): 2014. doi:10.1038/mtm.2013.5), myelin basic promoter (MBP) (see e.g., von Jonquieres, G., Mersmann, N., Klugmann, C. B., Harasta, A. E., Lutz, B., Teahan, O., et al. (2013). Glial promoter selectivity following AAV-delivery to the immature brain. PLoS One 8 (6), e65646. doi: 10.1371/journal.pone.0065646), glial fibrillary acid protein (GFAP) for expression in astrocytes (see e.g., Smith-Arica, J. R., Morelli, A. E., Larregina, A. T., Smith, J., Lowenstein, P. R., Castro, M. G. (2000). Cell-type-specific and regulatable transgenesis in the adult brain: adenovirus-encoded combined transcriptional targeting and inducible transgene expression. Mol. Ther. 2 (6), 579-587. doi: 10.1006/mthe.2000.0215 and Lee, Y., Messing, A., Su, M., Brenner, M. (2008). GFAP promoter elements required for region-specific and astrocyte-specific expression. Glia 56 (5), 481-493. doi: 10.1002/glia.20622), human myelin associated glycoprotein promoter (full-length or truncated) (see e.g., von Jonquieres, G., Frohlich, D., Klugmann, C. B., Wen, X., Harasta, A. E., Ramkumar, R., et al. (2016). Recombinant human myelin-associated glycoprotein promoter drives selective AAV-mediated transgene expression in oligodendrocytes. Front. Mol. Neurosci. 9, 13. doi: 10.3389/fnmol.2016.00013), F4/80 promoter (see e.g., Rosario, A. M., Cruz, P. E., Ceballos-Diaz, C., Strickland, M. R., Siemienski, Z., Pardo, M., et al. (2016). Microglia-specific targeting by novel capsid-modified AAV6 vectors. Mol. Ther. Methods Clin. Dev. 3, 16026. doi: 10.1038/mtm.2016.26), phosphate-activated glutaminase (PAG) or the vesicular glutamate transporter (vGLUT) promoter (for about 90% glutamatergic neuron-specific expression) (see e.g., Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j.brainres.2007.01.125), glutamic acid decarboxylase (GAD) promoter (for about 90% GABAergic neuron-specific expression) (see e.g., Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j.brainres.2007.01.125), MeCP2 promoter (see e.g., Gray et al. Hum Gene Ther. 2011 September; 22(9):1143-53. doi: 10.1089/hum.2010.245), and retinoblastoma gene promoter (see e.g., Jiang et al., J. Biol. Chem. 2001. 276, 593-600).

Suitable constitutive promoters include, but are not limited to CMV, RSV, SV40, EF1alpha, CAG, and beta-actin.

A AVs with Reduced Non-CNS Cell Specificity

In some embodiments, the n-mer insert(s) and/or P-motif(s) are inserted into an AAV polypeptide (e.g., an AAV capsid polypeptide) that has reduced specificity (or no detectable, measurable, or clinically relevant interaction) for one or more non-CNS cell types. Exemplary non-CNS cell types include, but are not limited to, liver, kidney, lung, heart, spleen, muscle (skeletal and cardiac), bone, immune, stomach, intestine, eye, skin cells and the like. In some embodiments, the non-CNS cells are liver cells.

In certain example embodiments, the AAV capsid polypeptide is an engineered AAV capsid polypeptide having reduced or eliminated uptake in a non-CNS cell as compared to a corresponding wild-type AAV capsid polypeptide.

In certain example embodiments, the non-CNS cell is a liver cell.

In certain example embodiments, the wild-type capsid polypeptide is an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, the engineered AAV capsid polypeptide comprises one or more mutations that result in reduced or eliminated uptake in a non-CNS cell. In certain example embodiments, the engineered AAV capsid polypeptide comprises one or more mutations that result in reduced or eliminated uptake in a non-CNS cell as compared to a CNS cell. In certain example embodiments, the engineered AAV capsid polypeptide comprises one or more mutations that result in increased update in a CNS cell as compared to a non-CNS cell, where such a mutation is not the inclusion of a targeting moiety of the present invention, but a mutation that is in addition to such a targeting moiety. In some embodiments, the non-CNS cell is a liver cell or a dorsal root ganglion neuron.

In certain example embodiments, the one or more mutations are in position 267, in position 269, in position 272, in position 504, in position 505, in position 585, in position 590, or any combination thereof in the AAV9 capsid polypeptide (SEQ ID NO: 1) or in one or more positions corresponding thereto in a non-AAV9 capsid polypeptide.

In certain example embodiments, the non-AAV9 capsid polypeptide is an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

In certain example embodiments, the mutation in position 267 in the AAV9 capsid polypeptide (SEQ ID NO: 1) or position corresponding thereto in a non-AAV9 capsid polypeptide is a G or X mutation to A, wherein X is any amino acid.

In certain example embodiments, the mutation in position 269 in the AAV9 capsid polypeptide (SEQ ID NO: 1) or position corresponding thereto in a non-AAV9 capsid polypeptide is an S or X to T mutation, wherein X is any amino acid.

In certain example embodiments, the mutation in position 272 in the AAV9 capsid polypeptide (SEQ ID NO: 1) or position corresponding thereto in a non-AAV9 capsid polypeptide is an N or to A mutation, wherein X is any amino acid. See also, e.g., International Patent Application Publication No. WO2018119330.

In certain example embodiments, the mutation in position 504 in the AAV9 capsid polypeptide (SEQ ID NO: 1) or position corresponding thereto in a non-AAV9 capsid polypeptide is a G or X to A mutation, wherein X is any amino acid.

In certain example embodiments, the mutation in position 505 in the AAV9 capsid polypeptide (SEQ ID NO: 1) or position corresponding thereto in a non-AAV9 capsid polypeptide is a P or X to A mutation, wherein X is any amino acid.

In certain example embodiments, the mutation in position 585 in the AAV9 capsid polypeptide (SEQ ID NO: 1) or position corresponding thereto in a non-AAV9 capsid polypeptide is an R or X to Q mutation, wherein X is any amino acid.

In certain example embodiments, the mutation in position 590 in the AAV9 capsid polypeptide (SEQ ID NO: 1) or position corresponding thereto in a non-AAV9 capsid polypeptide is a Q or X to A mutation, wherein X is any amino acid.

In certain example embodiments, the engineered AAV capsid polypeptide is an engineered AAV9 capsid polypeptide comprising a mutation at position 267, position 269 or both of a wild-type AAV9 capsid polypeptide (SEQ ID NO: 1), wherein the mutation at position 267 is a G to A mutation and wherein the mutation at position 269 is an S to T mutation.

In certain example embodiments, the engineered AAV capsid polypeptide is an engineered AAV9 capsid polypeptide comprising a mutation at position 590 of a wild-type AAV9 capsid polypeptide (SEQ ID NO: 1), wherein the mutation at position 509 is a Q to A mutation.

In certain example embodiments, the engineered AAV capsid polypeptide is an engineered AAV9 capsid polypeptide comprising a mutation at position 504, position 505, or both of a wild-type AAV9 capsid polypeptide (SEQ ID NO: 1), wherein the mutation at position 504 is a G to A mutation and wherein the mutation at position 505 is a P to A mutation.

In some embodiments, the AAV capsid polypeptide in which the n-mer insert(s) and/or P motif(s), and/or double valine motifs are inserted are 80-100 (e.g., 80, 81, 82, 83, 84, 85, 86, 87, 88, 89, 90, 91, 92, 93, 94, 95, 96, 97, 98, 99, to/or 100) percent identical to SEQ ID NO: 4 or SEQ ID NO: 5 of International Patent Application Publication WO 2019/217911, which is incorporated by reference as if expressed in its entirety herein. These sequences are also incorporated herein as SEQ ID NOS: 330 and 331 respectively. It will be appreciated that when considering variants of these AAV9 capsid proteins with reduced liver specificity, that residues 267 and/or 269 must contain the relevant mutations or equivalents.

In some embodiments, the AAV capsid polypeptide in which the in which the n-mer insert(s), such as an n-mer insert containing a P-motif and/or double valine motif, is/are inserted can be 80-100 (e.g., 80, 81, 82, 83, 84, 85, 86, 87, 88, 89, 90, 91, 92, 93, 94, 95, 96, 97, 98, 99, to/or 100) percent identical to any of those described in Adachi et al., (Nat. Comm. 2014. 5:3075, DOI: 10.1038/ncomms4075) that have reduced specificity for a non-CNS cell, particularly a liver cell. Adachi et al., (Nat. Comm. 2014. 5:3075, DOI: 10.1038/ncomms4075) is incorporated by reference herein as if expressed in its entirety.

In some embodiments, the modified AAV can have about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent or fold reduction in specificity for a non-CNS cells as compared to a wild-type AAV or control. In some embodiments, the modified AAV can have no measurable or detectable uptake and/or expression in one or more non-CNS cells.

In some embodiments, the AAV capsid protein in which the n-mer insert(s) and/or P motif(s), and/or double valine motifs are inserted are 80-100 (e.g., 80, 81, 82, 83, 84, 85, 86, 87, 88, 89, 90, 91, 92, 93, 94, 95, 96, 97, 98, 99, to/or 100) percent identical to any one of those set forth in International Patent Application Pub. WO 2018119330.

### Methods of Generating Engineered AAV Capsids

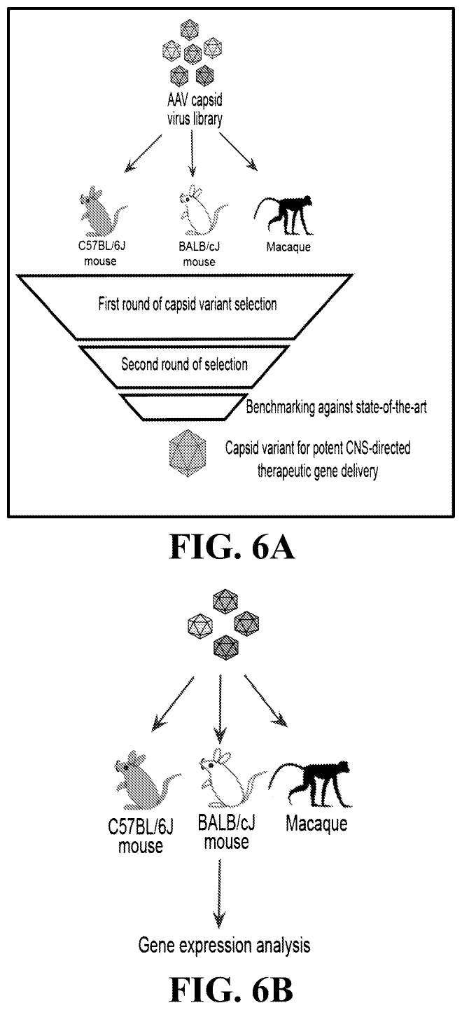

Also provided herein are methods of generating engineered AAV capsids. The engineered AAV capsid variants can be variants of wild-type AAV capsids. FIGS. 6A-8 can illustrate various embodiments of methods capable of generating engineered AAV capsids described herein. Generally, an AAV capsid library can be generated by expressing engineered capsid vectors each containing an engineered AAV capsid polynucleotide previously described in an appropriate AAV producer cell line. See e.g., FIG. 8. It will be appreciated that although FIG. 8 shows a helper-dependent method of AAV particle production, it will be appreciated that this can be done via a helper-free method as well. This can generate an AAV capsid library that can contain one more desired cell-specific engineered AAV capsid variant. As shown in FIG. 6 the AAV capsid library can be administered to various non-human animals for a first round of mRNA-based selection. As shown in FIG. 1, the transduction process by AAVs and related vectors can result in the production of an mRNA molecule that is reflective of the genome of the virus that transduced the cell. As is at least demonstrated in the Examples herein, mRNA based-selection can be more specific and effective to determine a virus particle capable of functionally transducing a cell because it is based on the functional product produced as opposed to just detecting the presence of a virus particle in the cell by measuring the presence of viral DNA.

After first-round administration, one or more engineered AAV virus particles having a desired capsid variant can then be used to form a filtered AAV capsid library. Desirable AAV virus particles can be identified by measuring the mRNA expression of the capsid variants and determining which variants are highly expressed in the desired cell type(s) as compared to non-desired cells type(s). Those that are highly expressed in the desired cell, tissue, and/or organ type are the desired AAV capsid variant particles. In some embodiments, the AAV capsid variant encoding polynucleotide is under control of a tissue-specific promoter that has selective activity in the desired cell, tissue, or organ.

The engineered AAV capsid variant particles identified from the first round can then be administered to various non-human animals. In some embodiments, the animals used in the second round of selection and identification are not the same as those animals used for first round selection and identification. Similar to round 1, after administration the top expressing variants in the desired cell, tissue, and/or organ type(s) can be identified by measuring viral mRNA expression in the cells. The top variants identified after round two can then be optionally barcoded and optionally pooled. In some embodiments, top variants from the second round can then be administered to a non-human primate to identify the top cell-specific variant(s), particularly if the end use for the top variant is in humans. Administration at each round can be systemic.

In some embodiments, the method of generating an AAV capsid variant can include the steps of: (a) expressing a vector system described herein that contains an engineered AAV capsid polynucleotide in a cell to produce engineered AAV virus particle capsid variants; (b) harvesting the engineered AAV virus particle capsid variants produced in step (a); (c) administering engineered AAV virus particle capsid variants to one or more first subjects, wherein the engineered AAV virus particle capsid variants are produced by expressing an engineered AAV capsid variant vector or system thereof in a cell and harvesting the engineered AAV virus particle capsid variants produced by the cell; and (d) identifying one or more engineered AAV capsid variants produced at a significantly high level by one or more specific cells or specific cell types in the one or more first subjects. In this context, “significantly high” can refer to a titer that can range from between about 2×10¹¹ to about 6×10¹² vector genomes per 15 cm dish.

The method can further include the steps of: (e) administering some or all engineered AAV virus particle capsid variants identified in step (d) to one or more second subjects; and (f) identifying one or more engineered AAV virus particle capsid variants produced at a significantly high level in one or more specific cells or specific cell types in the one or more second subjects. The cell in step (a) can be a prokaryotic cell or a eukaryotic cell. In some embodiments, the administration in step (c), step (e), or both is systemic. In some embodiments, one or more first subjects, one or more second subjects, or both, are non-human mammals. In some embodiments, one or more first subjects, one or more second subjects, or both, are each independently selected from the group consisting of: a wild-type non-human mammal, a humanized non-human mammal, a disease-specific non-human mammal model, and a non-human primate.

### Engineered Vectors and Vector Systems

Also provided herein are vectors and vector systems that can contain one or more of the engineered polynucleotides, (e.g., an AAV capsid polynucleotide) described herein. As used in this context, engineered viral (e.g., AAV) capsid polynucleotides refers to any one or more of the polynucleotides described herein capable of encoding an engineered viral (e.g., AAV) capsid as described elsewhere herein and/or polynucleotide(s) capable of encoding one or more engineered viral (e.g., AAV) capsid proteins described elsewhere herein. Further, where the vector includes an engineered viral (e.g., AAV) capsid polynucleotide described herein, the vector can also be referred to and considered an engineered vector or system thereof although not specifically noted as such. In embodiments, the vector can contain one or more polynucleotides encoding one or more elements of an engineered viral (e.g., AAV) capsid described herein. The vectors can be useful in producing bacterial, fungal, yeast, plant cells, animal cells, and transgenic animals that can express one or more components of the engineered viral (e.g., AAV) capsid described herein. Within the scope of this disclosure are vectors containing one or more of the polynucleotide sequences described herein. One or more of the polynucleotides that are part of the engineered viral (e.g., AAV) capsid and system thereof described herein can be included in a vector or vector system.

In some embodiments, the vector can include an engineered viral (e.g., AAV) capsid polynucleotide having a 3′ polyadenylation signal. In some embodiments, the 3′ polyadenylation is an SV40 polyadenylation signal. In some embodiments the vector does not have splice regulatory elements. In some embodiments, the vector includes one or more minimal splice regulatory elements. In some embodiments, the vector can further include a modified splice regulatory element, wherein the modification inactivates the splice regulatory element. In some embodiments, the modified splice regulatory element is a polynucleotide sequence sufficient to induce splicing, between a rep protein polynucleotide and the engineered viral (e.g., AAV) capsid protein variant polynucleotide. In some embodiments, the polynucleotide sequence can be sufficient to induce splicing is a splice acceptor or a splice donor. In some embodiments, the viral (e.g., AAV) capsid polynucleotide is an engineered viral (e.g., AAV) capsid polynucleotide as described elsewhere herein.

The vectors and/or vector systems can be used, for example, to express one or more of the engineered viral (e.g., AAV) capsid polynucleotides in a cell, such as a producer cell, to produce engineered viral (e.g., AAV) particles containing an engineered viral (e.g., AAV) capsid described elsewhere herein. Other uses for the vectors and vector systems described herein are also within the scope of this disclosure. In general, and throughout this specification, the term is a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include adeno-associated viruses, and types of such vectors can also be selected for targeting particular types of cells, such as those engineered viral (e.g., AAV) vectors containing an engineered viral (e.g., AAV) capsid polynucleotide with a desired cell-specific tropism. These and other embodiments of the vectors and vector systems are described elsewhere herein.

In some embodiments, the vector can be a bicistronic vector. In some embodiments, a bicistronic vector can be used for one or more elements of the engineered viral (e.g., AAV) capsid system described herein. In some embodiments, expression of elements of the engineered viral (e.g., AAV) capsid system described herein can be driven by a suitable constitutive or tissue specific promoter. Where the element of the engineered viral (e.g., AAV) capsid system is an RNA, its expression can be driven by a Pol III promoter, such as a U6 promoter. In some embodiments, the two are combined.

### Cell-Based Vector Amplification and Expression

Vectors can be designed for expression of one or more elements of the engineered targeting moieties, polypeptides, viral (e.g., AAV) capsid system described herein (e.g., nucleic acid transcripts, proteins, enzymes, and combinations thereof), etc. in a suitable host cell. In some embodiments, the suitable host cell is a prokaryotic cell. Suitable host cells include, but are not limited to, bacterial cells, yeast cells, insect cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U20S, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M.A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements is described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byme and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the a-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of an engineered targeting moiety, polypeptide, viral (e.g., AAV) capsid system so as to drive expression of the one or more elements of the engineered targeting moiety, polypeptide, viral (e.g., AAV) capsid system described herein.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more elements of an engineered targeting moiety, polypeptide, viral (e.g., AAV) capsid system described herein are introduced into a host cell such that expression of the elements of the engineered delivery system described herein direct formation of an engineered targeting moiety, polypeptide, viral (e.g., AAV) capsid system described herein (including but not limited to an engineered gene transfer agent particle, which is described in greater detail elsewhere herein). For example, different elements of the engineered targeting moiety, polypeptide, viral (e.g., AAV) capsid system described herein can each be operably linked to separate regulatory elements on separate vectors. RNA(s) of different elements of the engineered delivery system described herein can be delivered to an animal or mammal or cell thereof to produce an animal or mammal or cell thereof that constitutively or inducibly or conditionally expresses different elements of the engineered targeting moiety, polypeptide, viral (e.g., AAV) capsid system described herein that incorporates one or more elements of the engineered targeting moiety, polypeptide, viral (e.g., AAV) capsid system described herein or contains one or more cells that incorporates and/or expresses one or more elements of the engineered targeting moiety, polypeptide, viral (e.g., AAV) capsid system described herein.

In some embodiments, two or more of the elements expressed from the same or different regulatory element(s), can be combined in a single vector, with one or more additional vectors providing any components of the system not included in the first vector. Engineered targeting moiety, polypeptide, viral (e.g., AAV) capsid system polynucleotides that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more engineered targeting moiety, polypeptide, viral (e.g., AAV) capsid polypeptides, embedded within one or more intron sequences (e.g., each in a different intron, two or more in at least one intron, or all in a single intron). In some embodiments, the engineered targeting moiety, polypeptide, viral (e.g., AAV) capsid polynucleotides can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In embodiments, the polynucleotides and/or vectors thereof described herein (such as the engineered targeting moiety, polypeptide, viral (e.g., AAV) capsid polynucleotides of the present invention) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue and/or cells of interest, such as CNS cells and/or particular cell types therein (e.g., neurons and/or supporting cells (e.g., Schwan, astrocytes, glial cells, microglial cells, and/or the like). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the J3-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. In some embodiments, the regulated promoter is a tissue specific promoter as previously discussed elsewhere herein. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, CNS tissue and cell specific promoters.

Suitable neuronal tissue/cell specific promoters include, but are not limited to, GFAP promoter (astrocytes), SYN1 promoter (neurons), and NSE/RU5′ (mature neurons).

Other suitable CNS specific promoters can include, but are not limited to, neuroactive peptide cholecystokinin (CCK) (see e.g., Chhatawl et al. Gene Therapy volume 14, pages 575-583(2007)), a brain specific DNA MiniPromoter (such as any of those identified for brain or pan-neronal expression as in de Leeuw et al. Mol. Therapy. 1(5): 2014. doi:10.1038/mtm.2013.5), myelin basic promoter (MBP) (see e.g., von Jonquieres, G., Mersmann, N., Klugmann, C. B., Harasta, A. E., Lutz, B., Teahan, O., et al. (2013). Glial promoter selectivity following AAV-delivery to the immature brain. PLoS One 8 (6), e65646. doi: 10.1371/journal.pone.0065646), glial fibrillary acid protein (GFAP) for expression in astrocytes (see e.g., Smith-Arica, J. R., Morelli, A. E., Larregina, A. T., Smith, J., Lowenstein, P. R., Castro, M. G. (2000). Cell-type-specific and regulatable transgenesis in the adult brain: adenovirus-encoded combined transcriptional targeting and inducible transgene expression. Mol. Ther. 2 (6), 579-587. doi: 10.1006/mthe.2000.0215 and Lee, Y., Messing, A., Su, M., Brenner, M. (2008). GFAP promoter elements required for region-specific and astrocyte-specific expression. Glia 56 (5), 481-493. doi: 10.1002/glia.20622), human myelin associated glycoprotein promoter (full-length or truncated) (see e.g., von Jonquieres, G., Frohlich, D., Klugmann, C. B., Wen, X., Harasta, A. E., Ramkumar, R., et al. (2016). Recombinant human myelin-associated glycoprotein promoter drives selective AAV-mediated transgene expression in oligodendrocytes. Front. Mol. Neurosci. 9, 13. doi: 10.3389/fnmol.2016.00013), F4/80 promoter (see e.g., Rosario, A. M., Cruz, P. E., Ceballos-Diaz, C., Strickland, M. R., Siemienski, Z., Pardo, M., et al. (2016). Microglia-specific targeting by novel capsid-modified AAV6 vectors. Mol. Ther. Methods Clin. Dev. 3, 16026. doi: 10.1038/mtm.2016.26), phosphate-activated glutaminase (PAG) or the vesicular glutamate transporter (vGLUT) promoter (for about 90% glutamatergic neuron-specific expression) (see e.g., Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j.brainres.2007.01.125), glutamic acid decarboxylase (GAD) promoter (for about 90% GABAergic neuron-specific expression) (see e.g., Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j.brainres.2007.01.125), MeCP2 promoter (see e.g., Gray et al. Hum Gene Ther. 2011 September; 22(9):1143-53. doi: 10.1089/hum.2010.245), and retinoblastoma gene promoter (see e.g., Jiang et al., J. Biol. Chem. 2001. 276, 593-600).

Other tissue and/or cell specific promoters are discussed elsewhere herein and can be generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g. inducer binds a repressor bound to the promoter stimulating release of the promoter by the repressor or removal of a chemical repressor from the promoter environment).The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the components of the engineered targeting moiety, polypeptide, viral (e.g., AAV) capsid system described herein are typically placed under control of a plant promoter, i.e., a promoter operable in plant cells. The use of different types of promoters is envisaged. In some embodiments, inclusion of an engineered targeting moiety, polypeptide, viral (e.g., AAV) capsid system vector in a plant can be for AAV vector production purposes.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the engineered targeting moiety, polypeptide, viral (e.g., AAV) capsid system components are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the engineered targeting moiety, polypeptide, viral (e.g., AAV) capsid system are found in Kawamata et al., (1997) Plant Cell Physiol 38:792-803; Yamamoto et al., (1997) Plant J 12:255-65; Hire et al., (1992) Plant Mol Biol 20:207-18; Kuster et al., (1995) Plant Mol Biol 29:759-72; and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome)., such as a Light Inducible Transcriptional Effector (LITE) that direct changes in transcriptional activity in a sequence-specific manner. The components of a light inducible system may include one or more elements of the engineered targeting moiety, polypeptide, viral (e.g., AAV) capsid system described herein, a light-responsive cytochrome heterodimer (e.g., from Arabidopsis thaliana), and a transcriptional activation/repression domain. In some embodiments, the vector can include one or more of the inducible DNA binding proteins provided in PCT publication WO 2014/018423 and US Publications, 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the vector or system thereof can include one or more elements capable of translocating and/or expressing an engineered targeting moiety, polypeptide, viral (e.g., AAV) capsid polynucleotide to/in a specific cell component or organelle. Such organelles can include, but are not limited to, nucleus, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

One or more of the engineered targeting moiety, polypeptide, viral (e.g., AAV) capsid polynucleotides can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered targeting moiety, polypeptide, viral (e.g., AAV) capsid system polynucleotide such that the selectable marker polypeptide, when translated, is inserted between two amino acids between the N- and C-terminus of the engineered targeting moiety, polypeptide, viral (e.g., AAV) capsid polypeptide or at the N- and/or C-terminus of the engineered targeting moiety, polypeptide, viral (e.g., AAV) capsid polypeptide. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the engineered targeting moiety, polypeptide, viral (e.g., AAV) capsid system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the engineered AAV capsid system described herein via suitable linker, such as a glycine or glycine serine linkers as short as GS or GG up to (GGGGG)₃ (SEQ ID NO: 315) or (GGGGS)₃ (SEQ ID NO: 316). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the engineered targeting moiety, polypeptide, viral (e.g., AAV) capsid polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated engineered targeting moiety, polypeptide, viral (e.g., AAV) capsid polynucleotide(s) to specific cells, tissues, organs, etc.

### Cell-Free Vector and Polynucleotide Expression

In some embodiments, the polynucleotide encoding one or more features of the engineered targeting moiety, polypeptide, viral (e.g., AAV) capsid system can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding one or more embodiments of the engineered targeting moiety, polypeptide, viral (e.g., AAV) capsid system described herein can be codon optimized. In some embodiments, one or more polynucleotides contained in a vector (“vector polynucleotides”) described herein that are in addition to an optionally codon optimized polynucleotide encoding embodiments of the engineered targeting moiety, polypeptide, viral (e.g., AAV) capsid system described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.ojp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1):1-11.; as well as Codon usage in plant genes, Murray et al, Nucleic Acids Res. 1989 Jan. 25; 17(2):477-98; or Selection on the codon bias of chloroplast and cyanelle genes in diferent plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, CNS epithelial cells (including but not limited to the cells lining the brain ventricles), nerve cells (nerves, brain cells, spinal column cells, nerve support cells (e.g., astrocytes, glial cells, Schwann cells etc.), connective tissue cells of the CNS (fat and other soft tissue padding cells of the CNS such as the meninges), stem cells and other progenitor cells, CNS immune cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, CNS tissue and/or cells thereof. Such codon optimized sequences are within the ambit of the ordinary skilled artisan in view of the description herein. In some embodiments, the polynucleotide is codon optimized for a specific organ. Such organs include, but are not limited to, the brain. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those for derived from a particular organism, such as a plant or a mammal, including but not limited to human, or non-human eukaryote or animal or mammal as discussed herein, e.g., mouse, rat, rabbit, dog, livestock, or non-human mammal or primate.

Viral Vecto and/or Cargo Engineering for Reduced Immunogenicity and/or Toxicity

In some embodiments, the viral genome (such as an AAV genome) and/or cargo (e.g., cargo polynucleotide) is engineered to increase delivery and/or expression efficiency or to otherwise optimize delivery and/or expression efficiency so as to reduce immunogenicity and/or toxicity. See also e.g., Rapti and Grimm. of Front Immunol. 2021; 12: 753467, particularly at section 3.2.2.5 therein, and Domenger and Grimm. 2019. Human Molec Gen. 28(R1):R3-R14. It will be appreciated that one or more approaches discussed here and elsewhere herein can be combined.

In some embodiments, the engineered AAV is a self-complementary AAV (scAAV), which can have a favorable genome configuration with respect to efficiency.

In some embodiments, the engineered viral vector, such as an AAV viral vector, is engineered to have a cargo polynucleotide and/or genome that has a reduced number of CpG islands, which, without being bound by theory, can evade the adaptive and innate immune response by reducing TLR9 signaling. See also e.g., Faust et al., J Clin Invest (2013) 123:2994-3001 and Xiang et al., Mol Ther (2020) 28:771-83, the teachings of which can be adapted for use with the present invention.

In some embodiments, the engineered viral vector, such as an AAV viral vector, is engineered to include one or more short oligonucleotides in its genome that are configured to and/or capable of antagonizing TLR9 activation (referred to herein as TLR9i oligonucleotides), which, without being bound by theory can help the engineered viral particle evade TLR9 sensing and thus reduce immunogenicity. See e.g., Chan et al., Sci Transl Med. 2021 Feb. 10; 13(580), the teachings of which can be adapted for use with the present invention. In some embodiments, one or more TLR9i oligonucleotides (e.g., 1, 2, 3, 4, 5 or more) are incorporated into one or both of the inverted terminal repeats (ITRs) of a viral vector, such as an AAV viral vector. In some embodiments, the one or more TLR9i oligonucleotides are incorporated into the 5′ ITR. In some embodiments, the TLR9i oligonucleotides comprise 1 or more ODN repeats (e.g., 1, 2, 3, 4, 5 or more) that are optionally separated from each other via a linker polynucleotide. In some embodiments, the linker(s) is/are AAAAA. In some embodiments the ODN repeat comprises or consists of TAGGG. In some embodiments, the tTLR9i and/or ODN repeat comprises or consists of the sequence TAGGGTTAGGGTTAGGGTTAGGG (SEQ ID NO: 8582) or TTTAGGGTAGGGTAGGGTAGGG (SEQ ID NO: 8583). In some embodiments, the TLR9i oligonucleotides comprise or consist of the sequence TAGGGTAGGGTAGGGTAGGGAAAAATAGGGTAGGGTAGGGTAGG GAAAAATTAGGGTTAGGGTTAGGGTTAGGGAAAAA (SEQ ID NO: 8584). In some embodiments, the TLR9i oligonucleotides comprise or consist of the sequence TAGGGTAGGGTAGGGTAGGGAAAAATAGGGTAGGGTAGGGTAGG GAAAAATTTAGGGTTAGGGTTAGGGTTAGGGAAAAATGCAGCGGTAAGTTCCCA TCCAGGTTTTTTTGCAGCGGTAAGTTCCCATCCAGGTTTTTTGCAGCGGTAAGTTCC CATCCAGGTTTTT (SEQ ID NO: 8585). Other suitable TLR9i oligonucleotides are set forth in e.g., Chan et al., Sci Transl Med. 2021 Feb. 10; 13(580), particularly at Table S1, the teachings of which can be adapted for use with the present invention.

In some embodiments, the AAV vector is engineered to include a synthetic enhancer, promoter, or other cis acting regulatory element that is configured to optimize or otherwise control transcription of the genes they are associated with (e.g., including but not limited to a cargo polynucleotide). In some embodiments, the synthetic enhancer, promoter, or other cis acting regulatory element is positioned in the engineered AAV vector such that it is about 100 to about 1000 base pairs upstream of the gene or polynucleotide that it regulates (e.g., including but not limited to a cargo polynucleotide). In some embodiments, the synthetic enhancer, promoter, or other cis acting regulatory element contains one or more transcription factor binding sites, which are optionally engineered to bind specific transcription factors so as to control cargo expression temporally or spatially. For example, cell-specific transcription factors can be incorporated to spatially control expression. Exemplary spatial and temporal specific regulatory elements that can be incorporated are described in greater detail elsewhere herein. Additionally, promoter strength can be selected to further optimized polynucleotide expression of the AAV vector. Various promoters (strong and weak) are further described elsewhere herein and will be appreciated by one of ordinary skill in the art in view of the description herein. See also, e.g., Domenger and Grimm. 2019. Human Molec Gen. 28(R1):R3-R14, particularly at pages R4-R6, the teachings of which can be adapted for use with the present invention.. The specific combination of regulatory elements included can be used to fine tune and optimize cargo polynucleotide expression from a viral, e.g., AAV, vector or genome.

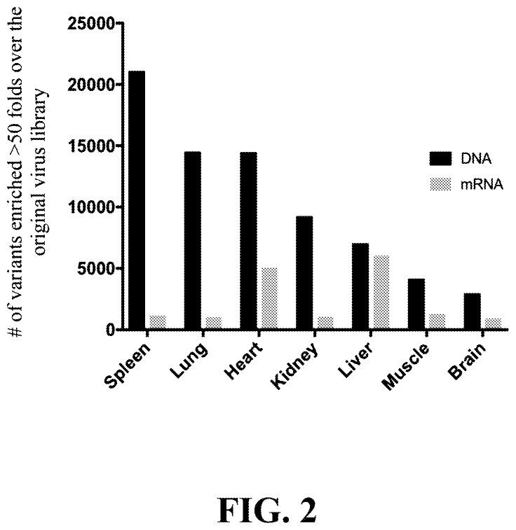

Other cis-acting elements, such as RNAi molecule binding sites or external stimuli responsive elements, can be incorporated into an engineered viral vector or viral vector genome, such as an AAV genome. By incorporating cell-type specific RNAi molecule binding sites, spatial expression of a cargo polynucleotide can be fine-tuned or optimized. Further, a synthetic or engineered RNAi molecule binding site can be included allowing control in a spatial and/or temporal manner by controlling where and/or when the synthetic or engineered RNAi molecule is present. In some embodiments, the polynucleotide encoding the synthetic RNAi molecule binding can also be incorporated into the viral vector genome such that it regulates a repressor or other regulatory element of the viral vector genome. In some embodiments, the RNAi molecule binding site(s) are incorporated into a viral vector genome within the 3′UTR of a cargo polynucleotide (e.g., a transgene) This is discussed in further detail elsewhere herein. In some embodiments, the viral vector, such as an AAV vector, is engineered to contain a LOV2 domain from Avena sativa that generates a blue light sensitive cargo polynucleotide. Thus, in this way blue light can be used to provide temporal and spatial control of transgene expression. See also e.g., Domenger and Grimm. 2019. Human Molec Gen. 28(R1):R3-R14, particularly at R7-R8 and FIG. 2, the teachings of which can be adapted for use with the present invention..

In some embodiments, the viral vector, e.g., AAV, is engineered to have one or more adverse structural elements deleted. Deleterious structural elements can be identified using a suitable screen strategy such as SMRT sequencing technology to identify vectors with adverse elements. In some embodiments, the adverse structural element is a shRNA, a hairpin sequence, or other secondary structure that mimics an ITR. See also e.g., Domenger and Grimm. 2019. Human Molec Gen. 28(R1):R3-R14, particularly at R9, the teachings of which can be adapted for use with the present invention.

Other exemplary modifications to reduce immunogenicity and/or toxicity are also described elsewhere herein.

Capsid Modifications for Improved Efficacy and/or Reduced Immunogenicity and/or Toxicity

In some embodiments, the polypeptide composition, such as a viral capsid or capsid polypeptide (e.g., AAV capsid or capsid polypeptide) of the present invention is engineered and/or rationally designed or evolved to contained one or more modifications (in addition to the n-mer motifs of the present invention) to modify and/or improve delivery, stability, efficacy, and/or reduce immunogenicity and/or toxicity of the protein composition, such as a viral capsid or capsid polypeptide (e.g., AAV capsid or capsid polypeptide) of the present invention. See e.g., Rapti and Grimm. of Front Immunol. 2021; 12: 753467, particularly at FIG. 2, Table 1 Section 3; Lam et al., J Pharm Sci, 86 (11)(1997), pp. 1250-1255, Le et al., J Control Release, 108 (1) (2005), pp. 161-177, Wonganan et al., Mol Pharm, 9 (7) (2011), pp. 78-92, Yao et al., Molecules, 22 (7) (2017), pp. 1-15, Zhao et al., J Virol, 90 (9) (2016), pp. 4262-4268, Gabriel et al. Hum Gene Ther Methods, 24 (2) (2013), pp. 80-93, Zhang et al., Biomaterials, 80 (2016), pp. 134-145, Mevel et al., Chem Sci, 11 (4) (2020), pp. 1122-1131, the teachings of which can be adapted for use with the present invention.

In some embodiments, the protein compositions, such as capsid protein(s) (e.g., AAV capsid polypeptides) of the present invention are PEGylated, which without being bound by theory, can mask the protein compositions, such as capsid protein(s) (e.g., AAV capsid polypeptides) of the present invention from antibodies. Suitable PEGylation of the protein compositions, such as capsid polypeptide(s) (e.g., AAV capsid polypeptides) of the present invention is described elsewhere herein.

In some embodiments, the protein compositions, such as capsid polypeptide(s) (e.g., AAV capsid polypeptides) of the present invention are engineered to reduce the number of oxidation susceptible residues, such as Met, Tyr, Trp, His, and/or Cys. In some embodiments, the protein compositions, such as capsid polypeptide(s) (e.g., AAV capsid polypeptides) of the present invention are engineered such that they contain one or more silent amino acid mutations (e.g., substitutions) that reduce the number of oxidation susceptible residues, such as Met, Tyr, Trp, His, and/or Cys. Without being bound by theory, such modifications can increase the stability, reduce degradation, increase half-life, and/or increase efficacy of the protein compositions, such as capsid polypeptide(s) (e.g., AAV capsid polypeptides) of the present invention.

In some embodiments, as is also further described herein, the protein compositions, such as capsid polypeptide(s) (e.g., AAV capsid polypeptides) of the present invention are encapsulated in a liposome, exosome, or other delivery vehicle. Without being bound by theory, such an approach can mask the protein compositions, such as capsid polypeptide(s) (e.g., AAV capsid polypeptides) of the present invention from immune components such as antibodies, thus reducing the immunogenicity of the composition.

In some embodiments, as is also further described herein, the protein compositions, such as capsid polypeptide(s) (e.g., AAV capsid polypeptides) of the present invention are cloaked via click labeling the polypeptide (e.g., capsid) to precisely tether oligonucleotides to the surface of the polypeptide composition (e.g., capsid) and associated or encapsulated with a lipid composition, (e.g., lipofectamine). See also e.g., Grimm et al., J Virol (2008) 82:5887-911. doi: 10.1128/JVI.00254-08, the teachings of which can be adapted for use with the present invention.

In some embodiments, the viral vector and/or polypeptide (e.g., capsid polypeptides) are selected, optimized and/or otherwise engineered to reduced immunogenicity. In some embodiments, and as discussed elsewhere herein, the serotype of the viral vector, such as AAV, can be selected to have a reduced immunogenicity in the recipient.

In some embodiments, the capsid polypeptide and/or capsid can be engineered and/or rationally designed or generated under a directed evolution approach to have reduced immunogenicity. In some embodiments, this is in addition or contemporaneous to any modification, engineering, selection, or directed evolution of proteins to have a specific tropism. See e.g., Rapti and Grimm. of Front Immunol. 2021; 12: 753467., particularly at Table 1 and Section 3/FIG. 2, the teachings of which can be adapted for use with the present invention.

As is also described herein, the immunogenicity of a viral capsid, particularly an AAV can be reduced, by one or more detargeting approaches, wherein the capsid or other component of the virial vector are modified to reduce delivery to or transgene/cargo expression in a non-target cell. In some embodiments, the capsid or capsid protein is modified at one or more residues to detarget a non-target cell, which can reduce the immunogenicity and/or toxicity of the viral particles. Exemplary modifications are described in greater detail elsewhere herein.

### Non-Viral Vectors and Carriers

In some embodiments, the vector is a non-viral vector or carrier. In some embodiments, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with an engineered targeting moiety, polypeptide, viral (e.g., AAV) capsid polynucleotide of the present invention and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid or polynucleotide molecule or composition that be attached to or otherwise interact with a polynucleotide to be delivered, such as an engineered targeting moiety, polypeptide, viral (e.g., AAV) capsid polynucleotide of the present invention.

### Naked Polynucleotides

In some embodiments one or more engineered targeting moiety, polypeptide, viral (e.g., AAV) capsid polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered targeting moiety, polypeptide, viral (e.g., AAV) capsid polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered targeting moiety, polypeptide, viral (e.g., AAV) capsid polynucleotide(s) of the present invention. In some embodiments, the naked polynucleotide can contain other nucleic acids and/or polynucleotides in addition to the engineered targeting moiety, polypeptide, viral (e.g., AAV) capsid polynucleotide(s) of the present invention. The naked polynucleotides can include one or more elements of a transposon system. Transposons and system thereof are described in greater detail elsewhere herein.

### Non-Viral Polynucleotide Vectors

In some embodiments, one or more of the engineered targeting moiety, polypeptide, viral (e.g., AAV) capsid polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g., minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In embodiments, the S/MAR sequence is located downstream of an actively transcribed polynucleotide (e.g., one or more engineered AAV capsid polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the engineered targeting moiety, polypeptide, viral (e.g., AAV) capsid polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the engineered targeting moiety, polypeptide, viral (e.g., AAV) capsid polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the engineered targeting moiety, polypeptide, viral (e.g., AAV) capsid polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, but are not limited to, Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

### Chemical Carriers

In some embodiments the engineered targeting moiety, polypeptide, viral (e.g., AAV) capsid polynucleotide(s) can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the engineered targeting moiety, polypeptide, viral (e.g. AAV) capsid polynucleotide(s) of the present invention), (2) those capable of targeting specific cells, (3) those capable of increasing delivery of the polynucleotide (such as the engineered targeting moiety, polypeptide, viral (e.g. AAV) capsid polynucleotide(s) of the present invention) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the engineered targeting moiety, polypeptide, viral (e.g., AAV) capsid system components described herein. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticuloendothelial system. In some embodiments, the inorganic particles can be optimized to protect an entrapped molecule from degradation. The Suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, particles, and materials, (e.g., supermagnetic iron oxide and magnetite), quantum dots, fullerenes (e.g., carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g., such as an engineered targeting moiety, polypeptide, viral (e.g., AAV) capsid polynucleotide of the present invention). In some embodiments, chemical non-viral carrier systems can include a polynucleotide such as the engineered targeting moiety, polypeptide, viral (e.g., AAV) capsid polynucleotide(s) of the present invention) and a lipid (such as a cationic lipid). These are also referred to in the art as lipoplexes. Other embodiments of lipoplexes are described elsewhere herein. In some embodiments, the non-viral lipid-based carrier can be a lipid nano emulsion. Lipid nano emulsions can be formed by the dispersion of an immisicible liquid in another stabilized emulsifying agent and can have particles of about 200 nm that are composed of the lipid, water, and surfactant that can contain the polynucleotide to be delivered (e.g., the engineered targeting moiety, polypeptide, viral (e.g., AAV) capsid polynucleotide(s) of the present invention). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g., polymer-based carriers and lipid-based carriers to functionalize these carriers). In some embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the engineered targeting moiety, polypeptide, viral (e.g., AAV) capsid polynucleotides of the present invention), polymethacrylate, and combinations thereof.

In some embodiments, the non-viral carrier can be configured to release an engineered delivery system polynucleotide that is associated with or attached to the non-viral carrier in response to an external stimulus, such as pH, temperature, osmolarity, concentration of a specific molecule or composition (e.g., calcium, NaCl, and the like), pressure and the like. In some embodiments, the non-viral carrier can be a particle that is configured includes one or more of the engineered AAV capsid polynucleotides describe herein and an environmental triggering agent response element, and optionally a triggering agent. In some embodiments, the particle can include a polymer that can be selected from the group of polymethacrylates and polyacrylates. In some embodiments, the non-viral particle can include one or more embodiments of the compositions microparticles described in US Pat. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the engineered targeting moiety, polypeptide, viral (e.g., AAV) capsid polynucleotide(s) of the present invention). Polymer-based systems are described in greater detail elsewhere herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as an engineered targeting moiety, polypeptide, viral (e.g., AAV) capsid polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the engineered targeting moiety, polypeptide, viral (e.g., AAV) capsid system described herein. The viral vector can be part of a viral vector system involving multiple vectors. In some embodiments, systems incorporating multiple viral vectors can increase the safety of these systems. Suitable viral vectors can include adenoviral-based vectors, adeno associated vectors, helper-dependent adenoviral (HdAd) vectors, hybrid adenoviral vectors, and the like. Other embodiments of viral vectors and viral particles produce therefrom are described elsewhere herein. In some embodiments, the viral vectors are configured to produce replication incompetent viral particles for improved safety of these systems.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2, 5, or 9. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261. The engineered AAV capsids can be included in an adenoviral vector to produce adenoviral particles containing said engineered AAV capsids.

In some embodiments the vector can be a helper-dependent adenoviral vector or system thereof. These are also referred to in the field as “gutless” or “gutted” vectors and are a modified generation of adenoviral vectors (see e.g., Thrasher et al. 2006. Nature. 443:E5-7). In embodiments of the helper-dependent adenoviral vector system one vector (the helper) can contain all the viral genes required for replication but contains a conditional gene defect in the packaging domain. The second vector of the system can contain only the ends of the viral genome, one or more engineered AAV capsid polynucleotides, and the native packaging recognition signal, which can allow selective packaged release from the cells (see e.g., Cideciyan et al. 2009. N Engl J Med. 361:725-727). Helper-dependent Adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered AAV capsid polynucleotides described herein. In some embodiments, the polynucleotide to be delivered via the viral particle produced from a helper-dependent adenoviral vector or system thereof can be up to about 38 kb. Thus, in some embodiments, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use in the engineered AAV capsid system of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the engineered AAV capsid system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the engineered targeting moiety, polypeptide, viral (e.g., AAV) capsid system of the present invention.

### Adeno Associated Vectors

In an embodiment, the engineered vector or system thereof can be an adeno-associated vector (AAV). See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the promoter can be a tissue specific promoter as previously discussed. In some embodiments, the tissue specific promoter can drive expression of an engineered capsid AAV capsid polynucleotide described herein.

The AAV vector or system thereof can include one or more polynucleotides that can encode one or more capsid polypeptides, such as the engineered AAV capsid polypeptides described elsewhere herein. The engineered capsid polypeptides can be capable of assembling into a protein shell (an engineered capsid) of the AAV virus particle. The engineered capsid can have a cell-, tissue- and/or organ-specific tropism.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 or any combinations thereof. In some embodiments, the AAV can be AAV1, AAV-2, AAV-5, AAV-9 or any combination thereof. One can select the AAV of the AAV with regard to the cells to be targeted; e.g., one can select AAV serotypes 1, 2, 5, 9 or a hybrid capsid AAV-1, AAV-2, AAV-5, AAV-9 or any combination thereof for targeting brain and/or neuronal cells; and one can select AAV-4 for targeting cardiac tissue; and one can select AAV-8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

It will be appreciated that while the different serotypes can provide some level of cell, tissue, and/or organ specificity, each serotype still is multi-tropic and thus can result in tissue-toxicity if using that serotype to target a tissue that the serotype is less efficient in transducing. Tus, in addition to achieving some tissue targeting capacity via selecting an AAV of a particular serotype, it will be appreciated that the tropism of the AAV serotype can be modified by an engineered AAV capsid described herein. As described elsewhere herein, variants of wild-type AAV of any serotype can be generated via a method described herein and determined to have a particular cell-specific tropism, which can be the same or different as that of the reference wild-type AAV serotype. In some embodiments, the cell, tissue, and/or specificity of the wild-type serotype can be enhanced (e.g., made more selective or specific for a particular cell type that the serotype is already biased towards). For example, wild-type AAV-9 is biased towards muscle and brain in humans (see e.g., Srivastava. 2017. Curr. Opin. Virol. 21:75-80.) By including an engineered AAV capsid and/or capsid polypeptide variant of wild-type AAV-9 as described herein, the bias for e.g., muscle (or other non-CNS tissue or cell) can be reduced or eliminated and/or the CNS tissue or cell specificity increased such that the muscle (or other non-CNS tissue or cell) specificity appears reduced in comparison, thus enhancing the specificity for the CNS tissue or cell as compared to the wild-type AAV-9. As previously mentioned, inclusion of an engineered capsid and/or capsid polypeptide n variant of a wild-type AAV serotype can have a different or more efficient and/or more specific tropism than the wild-type reference AAV serotype. For example, an engineered AAV capsid and/or capsid polypeptide variant of AAV-9 can have specificity for a tissue other than muscle or brain in humans or have heightened tropism for e.g., brain tissue as compared to wild-type AAV9.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same specificity issues as with the non-hybrid wild-type serotypes previously discussed.

Advantages achieved by the wild-type based hybrid AAV systems can be combined with the increased and customizable cell-specificity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV-9 serotype that is used to package a genome that contains components (e.g., rep elements) from an AAV-2 serotype. As with wild-type based hybrid AAVs previously discussed, the tropism of the resulting AAV particle will be that of the engineered AAV capsid.

A tabulation of certain wild-type AAV serotypes as to these cells can be found in Grimm, D. et al, J. Virol. 82: 5887-5911 (2008) reproduced below as Table 4. Further tropism details can be found in Srivastava. 2017. Curr. Opin. Virol. 21:75-80 as previously discussed.

TABLE 4

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature | 2500 | 100 | ND | ND | 222 | 2857 | ND | ND |
| DC | | | | | | | | |
| Mature DC | 2222 | 100 | ND | ND | 333 | 3333 | ND | ND |

In some embodiments, the AAV vector or system thereof is AAV rh.74 or AAV rh.10.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered AAV capsid polynucleotide(s)).

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vectors described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of an engineered AAV capsid system described herein are as used in the foregoing documents, such as WO 2014/093622 (PCT/US2013/074667) and are discussed in greater detail herein.

Virus Particle Production from Viral Vectors

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered AAV capsid polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the engineered AAV capsid polynucleotide(s)) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

The engineered AAV vectors and systems thereof described herein can be produced by any of these methods.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., engineered AAV capsid system transcripts, proteins, enzymes, mutant forms thereof, fusion proteins thereof, etc.), and virus particles (such as from viral vectors and systems thereof).

One or more engineered AAV capsid polynucleotides can be delivered using adeno associated virus (AAV), adenovirus or other plasmid or viral vector types as previously described, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some embodiments, doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of engineered AAV capsid system components (e.g., polynucleotides encoding engineered AAV capsid and/or capsid polypeptides) to cells via particles. The term “particle” as used herein, refers to any suitable sized particles for delivery of the engineered AAV capsid system components described herein. Suitable sizes include macro-, micro-, and nano-sized particles. In some embodiments, any of the of the engineered AAV capsid system components (e.g., polypeptides, polynucleotides, vectors, and combinations thereof described herein) can be attached to, coupled to, integrated with, otherwise associated with one or more particles or component thereof as described herein. The particles described herein can then be administered to a cell or organism by an appropriate route and/or technique. In some embodiments, particle delivery can be selected and be advantageous for delivery of the polynucleotide or vector components. It will be appreciated that in embodiments, particle delivery can also be advantageous for other engineered capsid system molecules and formulations described elsewhere herein.

### Engineered Virus Particles Including an Engineered Viral Capsid

Also described herein are engineered virus particles (also referred to here and elsewhere herein as “engineered viral particles”) that can contain an engineered viral (e.g., AAV) capsid as described in detail elsewhere herein. Viral particles with an engineered AAV capsid are referred to herein as engineered AAV particles. It will be appreciated that the engineered viral (e.g., AAV) particles can be adenovirus-based particles, helper adenovirus-based particles, AAV-based particles, or hybrid adenovirus-based particles that contain at least one engineered AAV capsid polypeptides as previously described. An engineered AAV capsid is one that that contains one or more engineered AAV capsid polypeptides as are described elsewhere herein. In some embodiments, the engineered AAV particles can include 1-60 engineered AAV capsid polypeptides described herein. In some embodiments, the engineered AAV particles can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid polypeptides. In some embodiments, the engineered AAV particles can contain 0-59 wild-type AAV capsid polypeptides. In some embodiments, the engineered AAV particles can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type AAV capsid polypeptides. The engineered AAV particles can thus include one or more n-mer inserts as is previously described.

The engineered AAV particle can include one or more cargo polynucleotides. Cargo polynucleotides are discussed in greater detail elsewhere herein. Methods of making the engineered AAV particles from viral and non-viral vectors are described elsewhere herein. Formulations containing the engineered virus particles are described elsewhere herein.

The engineered viral (e.g., AAV) capsid polynucleotides, other viral (e.g., AAV) polynucleotide(s), and/or vector polynucleotides can contain one or more cargo polynucleotides. The cargo polynucleotides can encode one or more polypeptides. Exemplary cargos are described in greater detail elsewhere herein. It will be appreciated that when a cargo polypeptide is described that its encoding polynucleotide can be a cargo polynucleotide described in this context. In some embodiments, the one or more cargo polynucleotides can be operably linked to the engineered viral (e.g., AAV) capsid polynucleotide(s) and can be part of the engineered viral (e.g., AAV) genome of the viral (e.g., AAV) system of the present invention. The cargo polynucleotides can be packaged into an engineered viral (e.g., AAV) particle, which can be delivered to, e.g., a cell. In some embodiments, the cargo polynucleotide can be capable of modifying a polynucleotide (e.g., gene or transcript) of a cell to which it is delivered. As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. Polynucleotide, gene, transcript, etc. modification includes all genetic engineering techniques including, but not limited to, gene editing as well as conventional recombinational gene modification techniques (e.g., whole or partial gene insertion, deletion, and mutagenesis (e.g., insertional and deletional mutagenesis) techniques.

### Engineered Cells and Organisms Expressing Said Engineered Viral Capsids

Described herein are engineered cells that can include one or more of the engineered targeting moieties, polypeptides, viral (e.g., AAV) capsid polynucleotides, polypeptides, vectors, and/or vector systems described in greater detail elsewhere herein. In some embodiments, one or more of the engineered viral (e.g., AAV) capsid polynucleotides can be expressed in the engineered cells. In some embodiments, the engineered cells can be capable of producing engineered viral (e.g., AAV) capsid polypeptides and/or engineered viral (e.g., AAV) capsid particles that are described elsewhere herein. Also described herein are modified or engineered organisms that can include one or more engineered cells described herein. The engineered cells can be engineered to express a cargo molecule (e.g., a cargo polynucleotide) dependently or independently of an engineered viral (e.g., AAV) capsid polynucleotide as described elsewhere herein.

A wide variety of animals, plants, algae, fungi, yeast, etc. and animal, plant, algae, fungus, yeast cell or tissue systems may be engineered to express one or more nucleic acid constructs of the engineered targeting moiety, polypeptide, vector, viral (e.g., AAV) capsid system described herein using various transformation methods mentioned elsewhere herein. This can produce organisms that can produce engineered targeting moiety, polypeptide, vector, viral (e.g., AAV) capsid particles, such as for production purposes, engineered targeting moiety, polypeptide, vector, viral (e.g., AAV) capsid design and/or generation, and/or model organisms. In some embodiments, the polynucleotide(s) encoding one or more components of the engineered targeting moiety, polypeptide, vector, viral (e.g., AAV) capsid system described herein can be stably or transiently incorporated into one or more cells of a plant, animal, algae, fungus, and/or yeast or tissue system. In some embodiments, one or more of engineered targeting moiety, polypeptide, vector, viral (e.g., AAV) capsid system polynucleotides are genomically incorporated into one or more cells of a plant, animal, algae, fungus, and/or yeast or tissue system. Further embodiments of the modified organisms and systems are described elsewhere herein. In some embodiments, one or more components of the engineered targeting moiety, polypeptide, vector, viral (e.g., AAV) capsid system described herein are expressed in one or more cells of the plant, animal, algae, fungus, yeast, or tissue systems.

### Engineered Cells

Described herein are various embodiments of engineered cells that can include one or more of the engineered targeting moiety, polypeptide, vector, viral (e.g., AAV) capsid system polynucleotides, polypeptides, vectors, and/or vector systems described elsewhere herein. In some embodiments, the cells can express one or more of the engineered targeting moiety, polypeptide, vector, viral (e.g., AAV) capsid polynucleotides and can produce one or more engineered targeting moiety, polypeptide, vector, viral (e.g., AAV) capsid particles, which are described in greater detail herein. Such cells are also referred to herein as “producer cells”. It will be appreciated that these engineered cells are different from “modified cells” described elsewhere herein in that the modified cells are not necessarily producer cells (i.e. they do not make engineered viral (e.g., AAV) particles) unless they include one or more of the engineered targeting moiety, polypeptide, viral (e.g., AAV) capsid polynucleotides, engineered targeting moiety, polypeptide, viral (e.g., AAV) capsid vectors or other vectors described herein that render the cells capable of producing an engineered viral (e.g., AAV) capsid particle or other particles described herein. Modified cells can be recipient cells of an engineered viral (e.g., AAV) capsid particles and can, in some embodiments, be modified by the engineered viral (e.g., AAV) capsid particle(s) and/or a cargo polynucleotide delivered to the recipient cell. Modified cells are discussed in greater detail elsewhere herein. The term modification can be used in connection with modification of a cell that is not dependent on being a recipient cell. For example, isolated cells can be modified prior to receiving an engineered targeting moiety, polypeptide, viral (e.g., AAV) capsid molecule.

In an embodiment, the invention provides a non-human eukaryotic organism; for example, a multicellular eukaryotic organism, including a eukaryotic host cell containing one or more components of an engineered delivery system described herein according to any of the described embodiments. In other embodiments, the invention provides a eukaryotic organism; preferably a multicellular eukaryotic organism, comprising a eukaryotic host cell containing one or more components of an engineered delivery system described herein according to any of the described embodiments. In some embodiments, the organism is a host of a virus (e.g., an AAV).

In particular embodiments, the plants, algae, fungi, yeast, etc., cells or parts obtained are transgenic plants, comprising an exogenous DNA sequence incorporated into the genome of all or part of the cells.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21(DE3), DL21(DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue (DE3), BLR, C41(DE3), C43(DE3), Lemo21 (DE3), Shuffle T7, ArcticExpress and ArticExpress (DE3).

The engineered cell can be a eukaryotic cell. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments the engineered cell can be a cell line. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CiR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X₆₃, YAC-1, YAR, and transgenic varieties thereof. Cell lines are available from a variety of sources known to those with skill in the art (see, e.g., the American Type Culture Collection (ATCC) (Manassas, Va.)).

In some embodiments, the engineered producer cell is a CNS cell, such as a neuron or supporting cell (e.g., a Schawan cell, astrocyte, glial cells, microglial cell and/or the like), a muscle cell (e.g., cardiac muscle, skeletal muscle, and/or smooth muscle), bone cell, blood cell, immune cell (including but not limited to B cells, macrophages, T-cells, CAR-T cells, and the like), kidney cells, bladder cells, lung cells, heart cells, liver cells, brain cells, neurons, skin cells, stomach cells, neuronal support cells, intestinal cells, epithelial cells, endothelial cells, stem or other progenitor cells, adrenal gland cells, cartilage cells, and combinations thereof.

In some embodiments, the engineered cell can be a fungus cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some embodiments, the engineered cell is a cell obtained from a subject. In some embodiments, the subject is a healthy or non-diseased subject. In some embodiments, the subject is a subject with a desired physiological and/or biological characteristic such that when an engineered targeting moiety, polypeptide, vector, viral (e.g., AAV) capsid particle is produced it can package one or more cargo polynucleotides that can be related to the desired physiological and/or biological characteristic and/or capable of modifying the desired physiological and/or biological characteristic. Thus, the cargo polynucleotides of the produced engineered viral (e.g., AAV) or other particles can be capable of transferring the desired characteristic to a recipient cell. In some embodiments, the cargo polynucleotides are capable of modifying a polynucleotide of the engineered cell such that the engineered cell has a desired physiological and/or biological characteristic.

In some embodiments, a cell transfected with one or more vectors described herein is used to establish a new cell line comprising one or more vector-derived sequences.

The engineered cells can be used to produce engineered targeting moieties, polypeptides, viral (e.g., AAV) capsid polynucleotides, vectors, and/or particles. In some embodiments, the engineered targeting moieties, polypeptides, viral (e.g., AAV) capsid polynucleotides, vectors, and/or particles are produced, harvested, and/or delivered to a subject in need thereof. In some embodiments, the engineered cells are delivered to a subject. Other uses for the engineered cells are described elsewhere herein. In some embodiments, the engineered cells can be included in formulations and/or kits described elsewhere herein.

The engineered cells can be stored short-term or long-term for use at a later time. Suitable storage methods are generally known in the art. Further, methods of restoring the stored cells for use (such as thawing, reconstitution, and otherwise stimulating metabolism in the engineered cell after storage) at a later time are also generally known in the art.

### Formulations

Component(s) of the engineered targeting moieties, polypeptides, viral (e.g., AAV) capsid system, engineered cells, engineered viral (e.g., AAV) particles, and/or combinations thereof can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 μg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 μg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, μL, mL, or L.

In embodiments, were engineered AAV capsid particles are included in the formulation, the formulation can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰, transducing units (TU)/mL of the engineered AAV capsid particles. In some embodiments, the formulation can be 0.1 to 100 mL in volume and can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰, transducing units (TU)/mL of the engineered AAV capsid particles.

### Pharmaceutically Acceptable Carriers and Auxiliary Ingredients and Agents

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to an amount of one or more of the polypeptides, polynucleotides, vectors, cells, engineered viral (e.g., AAV) capsids, viral (e.g., AAV) or other particles, nanoparticles, other delivery particles, and combinations thereof described herein, the pharmaceutical formulation can also include an effective amount of an auxiliary active agent, including but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

In embodiments where there is an auxiliary active agent contained in the pharmaceutical formulation in addition to the one or more of the polypeptides, polynucleotides, compositions, vectors, cells, virus particles, nanoparticles, other delivery particles, and combinations thereof described herein, amount, such as an effective amount, of the auxiliary active agent will vary depending on the auxiliary active agent. In some embodiments, the amount of the auxiliary active agent ranges from 0.001 micrograms to about 1 milligram. In other embodiments, the amount of the auxiliary active agent ranges from about 0.01 IU to about 1000 IU. In further embodiments, the amount of the auxiliary active agent ranges from 0.001 mL to about 1 mL. In yet other embodiments, the amount of the auxiliary active agent ranges from about 1% w/w to about 50% w/w of the total pharmaceutical formulation. In additional embodiments, the amount of the auxiliary active agent ranges from about 1% v/v to about 50% v/v of the total pharmaceutical formulation. In still other embodiments, the amount of the auxiliary active agent ranges from about 1% w/v to about 50% w/v of the total pharmaceutical formulation.

### Dosage Forms

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavemous, intrathecal, intravitreal, intracerebral, gingival, subgingival, intracerebroventricular, intra-arterial, intracarotid, intrathecal, intracisternal, subpial, intracerebroventricular, intraparenchymal, intracranial, subdural, subretinal, subconjunctival, intravitreal, intratympanic, intracochlear, intranasal, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, manitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavemous, gingival, subginigival, intrathecal, intravireal, intracerebral, and intracerebroventricular, and others) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents. Dosage forms for the eye can be adapted for topical administration to the eye, such as drops, suspensions, gels, hydrogels (e.g., contact lenses) and/or the like.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

In some embodiments, the pharmaceutical formulation and/or dosage form, is adapted for improved delivery and/or efficacy of a viral particle, particularly an AAV. In some embodiments, a viral particle or vector such as an AAV particle or vector, of the present invention is PEGylated. In some embodiments, the PEGlyation can improve the pharmacokinetics and/or pharmacodynamics of the viral particles, particularly AAV particles. In some embodiments, the engineered capsid polypeptides of the present invention, including but not limited to the engineered AAV capsid polypeptides are modified with one or more azide moieties which can then be orthogonally conjugated to one or more polyethylene glycols (PEGs) via click chemistry. In some embodiments, this approach can increase the stability (e.g., by 1-3 or more fold) and/or reduce immune system detection of the viral vectors (e.g., antibody recognition can be reduced by 0.1 to 2 or more fold). In some embodiments, the PEG used for PEGlyation is PEG 2000. PEGylated AAV2 particles via amine functionalities have been shown to protect the virus from neutralization and enable significant levels of gene expression upon re-administration without compromising the patient's immune system. See e.g., Harris and Chess. Le at al. Nat Rev Drug Discov, 2 (3) (2003), pp. 214-221, Brocchini et al., Nat Protoc, 1 (5) (2006), pp. 2241-2252, Gupta et al., J Cell Commun Signal, 13 (3) (2019), pp. 319-330, Pelegri-Oday et al., J. Am. Chem. Soc. 2014, 136, 41, 14323-14332, Le et al., J Control Release, 108 (1) (2005), pp. 161-177, and Lee et al. Biotechnol Bioeng, 92 (1) (2005), pp. 24-34, the teachings of which can be adapted for use with the present invention.

In some embodiments, the polypeptide compositions, viral vectors, viral polypeptides (e.g., capsid polypeptides and/or capsids), and/or viral particles are modified so as to improve transduction, stability, and/or other property of the polypeptide compositions, viral vectors, viral polypeptides, and/or viral particles, (in addition to inclusion of a n-mer motif described herein). In some embodiments, the modification(s) increase the stability and/or efficacy of the viral vectors, viral polypeptides (e.g., capsid polypeptides and/or capsids), and/or viral particles. In some embodiments, the capsid or capsid polypeptides thereof are modified by mutation of one or more serine, threonine, and/or lysine residues such that they are replaced with an alanine or arginine residues. In some embodiments, the modification is inclusion of an azide moiety in a viral capsid or capsid polypeptide of the present invention, such an AAV capsid or capsid polypeptide of the present invention. In some embodiments, the azide is introduced into the VP3 capsid domain. See e.g., Lam et al., J Pharm Sci, 86 (11) (1997), pp. 1250-1255, Le et al., J Control Release, 108 (1) (2005), pp. 161-177, Wonganan et al., Mol Pharm, 9 (7) (2011), pp. 78-92, Yao et al., Molecules, 22 (7) (2017), pp. 1-15, Zhao et al., J Virol, 90 (9) (2016), pp. 4262-4268, Gabriel et al. Hum Gene Ther Methods, 24 (2) (2013), pp. 80-93, Zhang et al., Biomaterials, 80 (2016), pp. 134-145, Mevel et al., Chem Sci, 11 (4) (2020), pp. 1122-1131, the teachings of which can be adapted for use with the present invention.

Peptide oxidation is a major cause of chemical instability and also sometimes linked to physical instability. For example, amino acids such as methionine, cysteine, histidine, tyrosine and tryptophan in peptides are susceptible to oxidation. More specifically viral capsid polypeptides can oxidize upon exposure to light and due to metal ion impurities in the raw materials and excipients, common to pharmaceutical formulations leading to a loss in functionality. In some embodiments, oxidation of the polypeptide compositions viral vectors, viral polypeptides (e.g., capsid polypeptides and/or capsids), and/or viral particles can be decreased and/or prevented by including free amino acids such as methionine and histidine and/or metal ion scavengers such as ethanol, EDTA and DTPA in a pharmaceutical formulation of the polypeptide compositions viral vectors, viral polypeptides (e.g., capsid polypeptides and/or capsids), and/or viral particles of the present invention. See e.g., Wang et al., Int J Pharm, 185 (1999), pp. 129-188, Evans et al., J Pharm Sci, 93 (10) (2004), pp. 2458-2475, Reinauer et al., J Pharm Sci, 109 (1) (2020), pp. 818-829, Kamerzell et al., Adv Drug Deliv Rev, 63 (13) (2011), pp. 1118-1159, Shah et al., J Pharm Sci, 107 (11) (2018), pp. 2789-2803, Shah et al., Int J Pharm, 547 (1-2) (2018), pp. 438-449, Tsai et al., Pharm Res An Off J Am Assoc Pharm Sci, 10 (5) (1993), pp. 649-659, Master et al., J Pharm Sci, 99 (5) (2010), pp. 2386-2398, and Lam et al., J Pharm Sci, 86 (11) (1997), pp. 1250-1255, the teachings of which can be adapted for use with the present invention.

Protein aggregation can cause an immunogenic response to protein compositions, including viral capsid compositions. In some embodiments, aggregation of proteins in a formulation, such as viral particles/vectors/capsids can be reduced by inclusion of one or more surfactants in the formulation. In some embodiments, a pharmaceutical formulation containing a protein composition, viral particle, viral capsid, and/or viral capsid polypeptide (e.g., an AAV capsid or capsid polypeptide) of the present invention contains one or more surfactants. In some embodiments, the surfactant is a nonionic surfactant. In some embodiments, the nonionic surfactant is a polysorbate (e.g., polysorbate 20, polysorbate 80). In some embodiments, the nonionic surfactant is poloxamer 188. Without being bound by theory, inclusion of a surfactant can also protect proteins against surface-induced damaged by competing with the proteins for adsorption sites on surfaces, of e.g., containers and delivery devices. See also e.g., Wang et al., Int J Pharm, 289 (1-2) (2005), pp. 1-30, Rodrigues et al., Pharm Res, 36 (2) (2019), pp. 1-20, Wright, J. F. Mol Ther, 12 (1)(2005), pp. 171-178, and Jones et al., ACS Symp Ser, 675 (1997), pp. 206-222, the teachings of which can be adapted for use with the present invention.

Salt can also affect the protein compositions, viral particles, viral vectors, viral capsids, and/or viral capsid proteins in a formulation. At low concentrations, salts affect electrostatic interactions in proteins. Therefore, this effect could be stabilizing when there are repulsive interactions leading to protein unfolding, or destabilizing when there are stabilizing salt bridges or ion pairs in the protein. At high salt concentrations, electrostatic interactions are saturated; the dominant effect of salt is on solvent properties of the solution. The stabilizing salts increase surface tension at water-protein interface and strengthen hydrophobic interactions by keeping hydrophobic groups away from water molecules, inducing preferential hydration of proteins. The salt effect strongly depends on the salt concentration and solution pH, as pH determines the charged state of ionizable amino acids in protein groups. In some embodiments, the salt composition and amounts are optimized for delivery and efficacy of the protein compositions, viral particles, viral vectors, viral capsids, and/or viral capsid proteins of the present invention.

Buffer and pH can influence conformational and colloidal stabilities of proteins, particularly viral capsid proteins. In some embodiments, the pharmaceutical formulation contains one or more buffers so as to optimize the pH of the formulation. The pH determines the net charge on the protein molecule and the nature of electrostatic interactions. Generally, the higher the net charge of the protein, the lower will be the aggregation propensity due to electrostatic repulsions, and higher will be the colloidal stability. In some embodiments, the pharmaceutical formulation contains a buffer optimized to the protein composition, viral particle, viral capsid, or capsid protein of the present invention such that the pH of the formulation is such that it results in a greater net charge of the protein as compared to an unbuffered formulation. In some embodiments, the buffer results in a pharmaceutical formulation of a protein composition, viral particle, viral capsid, or capsid protein of the present invention that has reduced aggregation and/or increased colloidal stability as compared to the same protein composition, viral particle, viral capsid, or capsid protein of the present invention in a formulation without said buffer. See also e.g., Marshall et al., Biochemistry, 50 (12)(2011), pp. 2061-2071, Kamihira et al., J Biol Chem, 278 (5)(2003), pp. 2859-2865, yun et al., Biophys J, 92 (11) (2007), pp. 4064-4077, Raman et al., Biochemistry, 44 (4) (2005), pp. 1288-1299, Jain and Udgaonkar et al., Biochemistry, 49 (35) (2010), pp. 7615-7624, and Klement et al., J Mol Biol, 373 (5) (2007), pp. 1321-1333, the teachings of which can be adapted for use with the present invention.

Osmolytes are small organic compounds cand can be included in a pharmaceutical formulation of the preset invention to stabilize proteins (e.g., the protein composition, viral particle, viral capsid, or capsid protein of the present invention) against denaturation and aggregation. Proteins in an aqueous solution exists in equilibrium between the folded (F) and unfolded (U) states. Without being bound by theory, stabilization by osmolytes occurs by a preferential exclusion mechanism where osmolytes shift the equilibrium towards the F-state. In some embodiments, a pharmaceutical formulation of the present invention includes one or more osmolytes. In some embodiments, the osmolyte(s) are sucrose, glycine, mannitol, histidine, dextrose, arginine, trehalose, lactose, or any combination thereof. In some embodiments, the osmolyte, such as a sugar (e.g., sucrose) can be used in a culture media used to produce viral particles, such as those of the present invention. In some embodiments, inclusion of the osmolyte in culture media during viral particle production increases viral particle yield by 0.1 to 5 fold or more. In some embodiments, the osmolyte incorporated into such a culture media is sucrose and optionally the concentration of the sucrose is about 0.2M. See also e.g., Deorkar and Thiyagarajan., Bio Pharm Int, 29 (10) (2016), pp. 26-30, Wang, W., Int J Pharm, 185 (1999), pp. 129-188, Barnett et al., J Phys Chem B, 120 (13)(2016), pp. 3318-3330, Amani et al., Protein J, 36 (2) (2017), pp. 147-153, Auton et al., Biophys Chem, 159 (1) (2011), pp. 90-99, Kendrick et al., Proc Natl Acad Sci USA, 94 (22) (1997), pp. 11917-11922, Timasheff, S. N., Proc Natl Acad Sci USA, 99 (15) (2002), pp. 9721-9726, Wlodarczyk et al., Eur J Pharm Biopharm, 131 (2018), pp. 92-98, and Rego et al., bioRxiv. Published online (2018), pp. 1-21, the teachings of which can be adapted for use with the present invention.

In some embodiments, the pH of the formulation is basic pH. Without being bound by theory, a basic pH can reduce disulfide formation and/or exchange, thus improving the stability and/or efficacy of the polypeptide compositions, such as capsid polypeptide(s) (e.g., AAV capsid polypeptide) of the present invention present in the formulation.

In some embodiments, as is also further described herein, the protein compositions, such as capsid polypeptide(s) (e.g., AAV capsid polypeptides) of the present invention can be encapsulated in a liposome, exosome, or other delivery vehicle. Without being bound by theory, such an approach can mask the protein compositions, such as capsid polypeptide(s) (e.g., AAV capsid polypeptides) of the present invention from immune components such as antibodies, thus reducing the immunogenicity of the composition.

### Kits

Also described herein are kits that contain one or more of the one or more of the polypeptides, polynucleotides, vectors, cells, or other components described herein and combinations thereof and pharmaceutical formulations described herein. In embodiments, one or more of the polypeptides, polynucleotides, vectors, cells, and combinations thereof described herein can be presented as a combination kit. As used herein, the terms “combination kit” or “kit of parts” refers to the compounds, or formulations and additional components that are used to package, screen, test, sell, market, deliver, and/or administer the combination of elements or a single element, such as the active ingredient, contained therein. Such additional components include but are not limited to, packaging, syringes, blister packages, bottles, and the like. The combination kit can contain one or more of the components (e.g., one or more of the one or more of the polypeptides, polynucleotides, vectors, cells, and combinations thereof) or formulation thereof can be provided in a single formulation (e.g., a liquid, lyophilized powder, etc.), or in separate formulations. The separate components or formulations can be contained in a single package or in separate packages within the kit. The kit can also include instructions in a tangible medium of expression that can contain information and/or directions regarding the content of the components and/or formulations contained therein, safety information regarding the content of the components(s) and/or formulation(s) contained therein, information regarding the amounts, dosages, indications for use, screening methods, component design recommendations and/or information, recommended treatment regimen(s) for the components(s) and/or formulations contained therein. As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory drive or CD-ROM or on a server that can be accessed by a user via, e.g., a web interface.

In one embodiment, the invention provides a kit comprising one or more of the components described herein. In some embodiments, the kit comprises a vector system and instructions for using the kit. In some embodiments, the vector system includes a regulatory element operably linked to one or more engineered targeting moiety, polypeptide, viral (e.g., AAV) delivery system polynucleotides, as described elsewhere herein and, optionally, a cargo molecule, which can optionally be operably linked to a regulatory element. The one or more engineered targeting moiety, polypeptide, viral (e.g., AAV) delivery system polynucleotides, can be included on the same or different vectors as a cargo molecule capable of being delivered by the engineered targeting moiety, polypeptide, viral (e.g., AAV) delivery system described herein in embodiments containing a cargo molecule within the kit.

In some embodiments, the kit comprises a vector system and instructions for using the kit. In some embodiments, the vector system comprises (a) a first regulatory element operably linked to a direct repeat sequence and one or more insertion sites for inserting one or more guide sequences up- or downstream (whichever applicable) of the direct repeat sequence, wherein when expressed, the guide sequence directs sequence-specific binding of a Cas9 CRISPR complex to a target sequence in a eukaryotic cell, wherein the Cas9 CRISPR complex comprises a Cas9 enzyme complexed with the guide sequence that is hybridized to the target sequence; and/or (b) a second regulatory element operably linked to an enzyme-coding sequence encoding said Cas9 enzyme comprising a nuclear localization sequence. Where applicable, a tracr sequence may also be provided. In some embodiments, the kit comprises components (a) and (b) located on the same or different vectors of the system. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the Cas9 enzyme comprises one or more nuclear localization sequences of sufficient strength to drive accumulation of said CRISPR enzyme in a detectable amount in the nucleus of a eukaryotic cell. In some embodiments, the CRISPR enzyme is a type V or VI CRISPR system enzyme. In some embodiments, the CRISPR enzyme is a Cas9 enzyme. In some embodiments, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9 (e.g., modified to have or be associated with at least one DD), and may include further alteration or mutation of the Cas9, and can be a chimeric Cas9. In some embodiments, the DD-CRISPR enzyme is codon-optimized for expression in a eukaryotic cell. In some embodiments, the DD-CRISPR enzyme directs cleavage of one or two strands at the location of the target sequence. In some embodiments, the DD-CRISPR enzyme lacks or substantially DNA strand cleavage activity (e.g., no more than 5% nuclease activity as compared with a wild-type enzyme or enzyme not having the mutation or alteration that decreases nuclease activity). In some embodiments, the first regulatory element is a polymerase III promoter. In some embodiments, the second regulatory element is a polymerase II promoter. In some embodiments, the guide sequence is at least 16, 17, 18, 19, 20, 25 nucleotides, or between 16-30, or between 16-25, or between 16-20 nucleotides in length.

### Methods of Use

### General Discussion

The compositions containing the CNS-specific targeting moieties described herein (e.g., the engineered targeting moiety system polynucleotides, polypeptides, vector(s), engineered cells, engineered viral (e.g., AAV) capsids, and viral and other particles) can be used generally to package and/or deliver one or more cargo polynucleotides or other cargo types to a recipient cell or cell population (including tissues, organs, and organsims). In some embodiments, delivery, is done in a cell-specific manner based upon the specificity of the targeting moiety(ies). In some embodiments, the cell-specificity is conferred via the n-mer insert(s) included in the targeting moiety as previously discussed. In some embodiments, delivery is done in cell-specific manner based upon the tropism of the engineered viral (e.g., AAV) capsid. In some embodiments, engineered targeting moiety(ies), polypeptides, viral (e.g., AAV) capsids, particles, viral (e.g., AAV) particles, compositions thereof, and/or cells discussed herein can be administered to a subject or a cell, tissue, and/or organ and facilitate the transfer and/or integration of the cargo polynucleotide to the recipient cell. In other embodiments, engineered cells capable of producing engineered targeting moiety(ies), polypeptides, viral (e.g., AAV) capsids, particles, viral (e.g., AAV) particles and/or compositions thereof can be generated from engineered targeting moiety system molecules (e.g., polynucleotides, vectors, and vector systems, etc.). In some embodiments, the engineered targeting moiety(ies), polypeptides, viral (e.g., AAV) capsids, particles, viral (e.g., AAV) particles and/or compositions thereof can be delivered to a subject or a cell, tissue, and/or organ. When delivered to a subject, they engineered delivery system molecule(s) can transform a subject's cell in vivo or ex vivo to produce an engineered cell that can be capable of making an engineered targeting moiety(ies), polypeptides, viral (e.g., AAV) capsids, particles, viral (e.g., AAV) particles and/or compositions thereof, which can be released from the engineered cell and deliver cargo molecule(s) to a recipient cell in vivo or produce personalized engineered polypeptides, viral (e.g., AAV) particles, and/or other particles for reintroduction into the subject from which the recipient cell was obtained. In some embodiments, an engineered cell can be delivered to a subject, where it can release produced engineered targeting moieties, polypeptides, viral (e.g., AAV) particles, and/or other particles such that they can then deliver a cargo (e.g., cargo polynucleotide(s)) to a recipient cell. These general processes can be used in a variety of ways to treat and/or prevent disease or a symptom thereof in a subject, generate model cells, generate modified organisms, provide cell selection and screening assays, in bioproduction, and in other various applications.

In some embodiments, the engineered targeting moieties, polypeptides, viral (e.g., AAV) particles, and/or other particles, polynucleotides, vectors, and systems thereof can be used to generate engineered AAV capsid variant libraries that can be mined for variants with a desired cell-specificity, such as CNS specificity. The description provided herein as supported by the various Examples can demonstrate that one having a desired cell-specificity in mind could utilize the present invention as described herein to obtain a capsid with the desired cell-specificity, such as CNS specificity.

### Therapeutics

In some embodiments, one or more molecules of the engineered delivery system, engineered targeting moieties, polypeptides, viral (e.g., AAV) particles, and/or other particles, polynucleotides, vectors, systems thereof, engineered cells, and/or formulations thereof described herein can be delivered to a subject in need thereof as a therapy for one or more diseases. In some embodiments, the disease to be treated is a genetic or epigenetic based disease. In some embodiments, the disease to be treated is not a genetic or epigenetic based disease. In some embodiments, one or more molecules of the engineered delivery system, engineered targeting moieties, polypeptides, viral (e.g., AAV) particles, and/or other particles, polynucleotides, vectors, and systems thereof, engineered cells, and/or formulations thereof described herein can be delivered to a subject in need thereof as a treatment or prevention (or as a part of a treatment or prevention) of a disease. It will be appreciated that the specific disease to be treated and/or prevented by delivery of an engineered cell and/or engineered can be dependent on the cargo molecule packaged into an engineered AAV capsid particle.

Generally, the compositions described herein can be used in a therapy for treating or preventing a CNS disease, disorder, or a symptom thereof. It will be appreciated that a CNS disease or disorder refers to any disease or disorder whose pathology involves or affects one or more cell types of the central nervous system. In some embodiments, the CNS disease or disorder is one whose primary pathology involves one or more cell types of the CNS. In some embodiments, one or more other cell types outside of the CNS are involved in the pathology of the CNS disease, such as a muscle cell or a peripheral nervous system cell. In some embodiments, the CNS disease or disorder can be caused by one or more genetic abnormalities. In some embodiments, the CNS disease or disorder is not caused by a genetic abnormality. Non-genetic causes of diseases include infection, cancer, physical trauma and others that will be appreciated by those of skill in the art. It also will be appreciated that gene modification approaches to treating disease can be applied to treat and/or prevent both genetic diseases and non-genetic diseases. For example, in the case of non-genetic diseases, a gene therapy approach can be used to modify the cause of the non-genetic disease (e.g., a cancer or infectious organism) such that the cause is no longer disease causing (e.g., by eliminating or rendering non-functional the cancer cells or infectious organism).

Exemplary CNS diseases and disorders include, without limitation, Friedreich's Ataxia, Dravet Syndrome, Spinocerebellar Ataxia Type 3, Niemann Pick Type C, Huntington's Disease, Pompe Disease, Myotonic Dystrophy Type 1, Gluta Deficiency Syndrome (De Vivo Syndrome), Tay-Sachs, Spinal Muscular Atrophy, Alzheimer's disease, Amyotrophic lateral sclerosis (ALS), Danon disease, Rett Syndrome, Angleman Syndrome, infantile neuronal dystorpy, Gaucher's disease, Krabbe disease, metachromatic leukodystrophy, Salla disease, Farber disease or Spinal Musular Atrophy with progressive myoclonic Epilepsy (also reffered to as Jankovic-Rivera syndrome, Unverricht-Lundborg disease, AADC deficiency, Parkinson's disease, Batten disease, a neuronal ceroid lipofuscinosis disease, giant axonal neuropathy, a mucopolysaccharidosis disease (e.g., Hurler syndrome, MPS III A-D), neurofibromatosis, a spinocerebellar ataxia disease, Sandoff disease, GM2 gangliosidosis, Canavan disease, Cockayne syndrome, a pain disease or disorder, a neuropathy or nerve damage, or any combination thereof. Others are described elsewhere herein and/or will be appreciated by those of ordinary skill in the art in view of the description provided herein.

In some embodiments, the compositions described herein can be used for treating or preventing an eye disease or disorder. It will be appreciated that an eye disease or disorder is a disease or disorder that has a pathology or clinical symptom that involves one or more cells or cell types of the eye, including but not limited to, the optic nerve, rods, cones, retinal cells (e.g., photoreceptors, bipolar cells, ganglion cells, horizontal cells, and amacrine cells), and/or the like. The eye disease or disorder can be of genetic or non-genetic origin. Exemplary eye diseases and disorders include, without limitation, Stargardt disease, a Leber's congenital amaurosis (LCA) (e.g., Leber's congenital amaurosis type 2, LEBER CONGENITALAMAUROSIS (LCA) ANDEARLY-ONSET SEVERE RETINALDYSTROPHY (EOSRD)), Choroideremia, a macular degeneration, diabetic retinopathy, a retinopathy, vitelliform macular dystrophy, a macular dystrophy, Sorsby's fundus dystrophy, cataracts, glaucoma, optic neuropathies, Marfan syndrome, myopia, polypoidal choroidal vasculopathies, retinitis pigmentosa, uveal melanoma, X-linked retinoschisis, pattern dystrophy, achromatopsia, Blue cone monochromatism, Bornholm eye disease, ADGUCA1A-associated COD/CORD, autosomal dominant PRPH2 associated CORD, X-linkedRPGR-associatedCOD/CORD, fundus albipunctatus, Enhanced S-conesyndrome, Bietti crystalline comeoretinaldystorphy, or any combination thereof.

In some embodiments, the compositions described herein can be used for treating or preventing an inner ear disease or disorder. It will be appreciated that an eye disease or disorder is a disease or disorder that has a pathology or clinical symptom that involves one or more cells or cell types of the ear, and more particularly the inner ear, including but not limited to, hair cells, pillar cells, Boettcher's cells, Claudius' cells, spiral ganglion neurons, and Deiters' cells (phalangeal cells). The inner ear disease or disorder can be of genetic or non-genetic origin. Exemplary inner ear disease and disorders include, without limitation, GJB-2 deafness, Jeryell and Lange-Nielsen syndrome, Usher syndrome, Alport syndrome, Branchio-oto-renal syndrome, Waardenburg syndrome, Pendred syndrome, Stickler syndrome, Treacher Collins syndrome, CHARGE syndrome, Norrie disease, Perrault syndrome, Autosomal dominant Nonsyndromic hearing loss, utosomal Recessive Nonsyndromic Hearing Loss, X-linked nonsyndromic hearing loss, an auditory neuropathy, a congenital hearing loss, or any combination thereof.

In some embodiments, the compositions comprising a CNS specific targeting moiety of the present invention and/or cargos that can be delivered by such compositions can be used to treat or prevent pain or a pain disease or disorder in a subject. In some embodiments, a cargo is capable of modulating sensitivity to or pain sensation/perception in a subject. It will be appreciated that depending on the disease or condition, it can be desirable to increase pain sensitivity or perception (e.g., in the case of disease where there is no pain sensitivity) or decrease pain sensitivity, sensation, and/or perception (e.g., neuropathies and others).

In some embodiments, the cargo molecule can treat or prevent a Pain disease or disorder or pain resulting from a disease or disorder. In some embodiments, the pain disease or disorder causes a deleterious insensitivity or lack of sensitivity to pain. In some embodiments, the pain is due to trauma or damage to a tissue and/or nerve(s)/neurons that can be the result of disease (e.g., ischemia, virus, etc.) or external trauma or mechanical pain (e.g., acute injury, surgical wounds and/or amputation, thermal exposure, etc. In some embodiments, the pain disease or disorder involves dysfunction of one or more neurons, ganglions, or other cells of the CNS and/or peripheral nervous system. In some embodiments, the disease or disorder generates inappropriate, hyper-, or other wise deleterious pain negatively impacting quality of life. Exemplary pain diseases or disorders include, without limitation, HSAN-1, HSAN-2, HSAN-3 (familial dysautonomia—pain free phenotype), HSAN-4 (CIPA), mutilated foot, erythermalagia, paroxysmal extreme pain, and other insensitivities to pain, neuropathic pain, other chronic pain, and/or the like. Exemplary targets for genetic modifications for pain modulation include those involved in signal transduction and/or conduction and/or synaptic transmission (TRPV1/2/3/4, P2XR3, TRPM8, TRPA1, P2RX3, P2RY, BDKRB1/2, Htr3A, ACCNs, TRPV4, TRPC/P, ACCN1/2, SCN10A, SCN11A, SCN1,3, 4A, SCN9A, KCNQ, (other K+ channel genes), NR1, 2, GRIA1-4, GRIC1-5, NK1R, CACNA1A-S, CACNA2D1; genes of the microglia (e.g., TLR2/4. P2RX4/7, CCL2, CX3CRN1), genes of the CNS (e.g., BDNF, OPRD1/K1/M1, CNR1, GABRs, TNF, PLA2), genes of the PNS (e.g., IL1/6/12/18, COX-2, NTRK1, NGF, GDNF, TNF, LIF, CCL2, CNR2), genes and/or any one or more of the SNPs set forth in Table 1 of Foulkes and Wood. PLOS Genetics. 2008. https://doi.org/10.1371/journal.pgen.1000086; any one or more genes associated with a heritable pain condition (e.g., SPTLC1, IkbKAP protein gene, CCT4, Nav1.7 gene); ion channel related genes (e.g., (SCN9A, CACNG2, ZSCAN20, SCN11A), Neurotransmission (OPRM1, COMT, PRKCA, SLCA4, MPZ, GCH1), Metabolism (GCH1, TF, CP, TFRC, ACO1, FXN, SLC11A2, B2M, BMP6), Immune Response (HLA-A, HLA-B, HLA-DQB1, HLA-DRB1, IL6, IL1R2, IL10, TNF-α, GFRA2, HMGB1P46), SCN9A (NaV1.7), SCN10A (NaV1.8) and SCN11A (NaV1.9), GAD, or any combination thereof. In some embodiments, the cargo is a glutamic acid decarboxylase (GAD) which can provide GABA to recue pain, such as neuropathic pain. In some embodiments, the pain-associated genes are modified using a CRISPRi approach (e.g., a cargo molecule can contain CRISPRi molecule(s). In some embodiments, the pain-associated genes are modified using a CRISPRi-KRAB approach. See also e.g., Wolfe et al., Pain Medicine, Volume 10, Issue 7, October 2009, Pages 1325-1330, Moreno A M, Glaucilene F C, Alemán F et al. Long-lasting analgesia via targeted in vivoepigenetic repression of Nav1.7. bioRxiv711812 (2019). https://www.biorxiv.org/content/10.1101/71, Foulkes and Wood. PLOS Genetics. 2008. https://doi.org/10.1371/journal.pgen.1000086, the teachings of which can be adapted for use with the present invention.

Genetic diseases that can be treated are discussed in greater detail elsewhere herein (see e.g., discussion on Gene-modification based-therapies below). Other diseases can include, but are not limited to, any of the following: cancer (such as glioblastoma or other brain or CNS cancers), Acubetivacter infections, actinomycosis, African sleeping sickness, AIDS/HIV, ameobiasis, Anaplasmosis, Angiostrongyliasis, Anisakiasis, Anthrax, Acranobacterium haemolyticum infection, Argentine hemorrhagic fever, Ascariasis, Aspergillosis, Astrovirus infection, Babesiosis, Bacterial meningitis, Bacterial pneumonia, Bacterial vaginosis, Bacteroides infection, balantidiasis, Bartonellosis, Baylisascaris infection, BK virus infection, Black Piedra, Blastocytosis, Blastomycosis, Bolivian hemorrhagic fever, Botulism, Brazillian hemmorhagic fever, brucellosis, Bubonic plague, Burkholderia infection, buruli ulcer, calicivirus invention, campylobacteriosis, Candidasis, Capillariasis, Carrion's disease, Cat-scratch disease, cellulitis, Chagas Disease, Chancroid, Chickenpox, Chikungunya, Chlamydia, Chlamydia pneumoniae, Cholera, Chromoblastomycosis, Chytridiomycosis, Clonochiasis, Clostridium difficile colitis, Coccidioidomycosis, Colorado tick fever, rhinovirus/coronavirus invection (common cold), Cretzfeldt-Jakob disease, Crimean-congo hemorrhagic fever, Cryptococcosis, Cryptosporidosis, Cutaneous larva migrans (CLM), cyclosporiasis, cysticercosis, cytomegalovirus infection, Dengue fever, Desmodesmus infection, Dientamoebiasis, Diptheria, Diphylobothriasis, Dracunculiasis, Ebola, Echinococcosis, Ehrlichiosis, Enterobiasis, Enterococcus infection, Enterovirus infection, Epidemic typhus, Erthemia Infectisoum, Exanthem subitum, Fasciolasis, Fasciolopsiasis, fatal familial insomnia, filarisis, Clostridum perfingens infection, Fusobacterium infection, Gas gangrene (clostridial myonecrosis), Geotrichosis, Gerstmann-Straussler-Scheinker syndrome, Giardasis, Glanders, Gnathostomiasis, Gonorrhea, Granuloma inguinales, Group A streptococcal infection, Group B streptococcal infection, Haemophilus influenzae infection, Hand, foot, and mouth disease, hanta virus pulmonary syndrome, heartland virus disease, Helicobacter pylori infection, hemorrhagi fever with renal syndrome, Hendra virus infection, Hepatitis (all groups A, B, C, D, E), hepes simplex, histoplasmosis, hookworm infection, human bocavirus infection, human ewingii erlichosis, Human granulocytic anaplasmosis, human metapneymovirus infection, human monocytic ehrlichosis, human papaloma virus, Hymenolepiasis, Epstein-Barr infection, mononucleosis, influenza, isoporisis, Kawasaki disease, Kingell kingae infection, Kuru, Lasas fever, Leginollosis (Legionnaires's disease and Potomac Fever), Leishmaniasis, Leprosy, Leptospirosis, Listeriosis, Lyme disease, lymphatic filariasis, lymphocytic choriomeningitis, Malaria, Marburg hemorrhagic feaver, measals, Middle East respiratory syndrome, Meliodosis, menigitis, Menigococcal disease, Metagonimiasis, Microsporidosis, Molluscum contagiosum, Monkeypox, Mumps, Murine typhus, Mycoplasma pneumonia, Mycoplasma genitalium infection, Mycetoma, Myiasis, Conjunctivitis, Nipah virus infection, Norovirus, Variant Creutzfeldt-Jakob disease, Nocardosis, Onchocerciasis, Opisthorchiasis, Paracoccidioidomycosis, Paragonimiasis, Pasteurellosis, Pdiculosisi capitis, Pediculosis corpis, Pediculosis pubis, pelvic inflammatory disease, pertussis, plague, pneumococcal infection, pneumocystis pneumonia, pneumonia, poliomyelitis, prevotella infection, primary amoebic menigoencephalitis, progressive multifocal leukoencephalopathy, Psittacosis, Qfever, rabies, relapsing fever, respiratory syncytial virus infection, rhinovirus infection, rickettsial infection, Rickettsialpox, Rift Valley Fever, Rocky Mountain Spotted Fever, Rotavirus infection, Rubella, Salmonellosis, SARS, Scabies, Scarlet fever, Schistosomiais, Sepsis, Shigellosis, Shingles, Smallpox, Sporotrichosisi, Staphlococcol infection (including MRSA), strongyloidiasis, subacute sclerosing panecephalitis, Syphillis, Taeniasis, tetanus, Trichophyton species infection, Tocariasis, Toxoplasmosis, Trachoma, Trichinosis, Trichuriasis, Tuberculosis, Tularemia, Typhoid Fever, Typhus Fever, Ureaplasma urealyticum infection, Valley fever, Venezuelan equine encephalitis, Venezuelan hemorrhagic fever, Vibrio species infection, Viral pneumonia, West Nile Fever, White Piedra, Yersinia pseudotuberculosis, Yersiniosis, Yellow fever, Zeaspora, Zika fever, Zygomycosis and combinationsthereof.

Other diseases and disorders or symptoms thereof that can be treated using embodiments of the present invention include, but are not limited to, endocrine diseases (e.g., Type I and Type II diabetes, gestational diabetes, hypoglycemia. Glucagonoma, Goitre, Hyperthyroidism, hypothyroidism, thyroiditis, thyroid cancer, thyroid hormone resistance, parathyroid gland disorders, Osteoporosis, osteitis deformans, rickets, ostomalacia, hypopituitarism, pituitary tumors, etc.), skin conditions of infections and non-infection origin, eye diseases of infectious or non-infectious origin, gastrointestinal disorders of infectious or non-infectious origin, cardiovascular diseases of infectious or non-infectious origin, brain and neuron diseases of infectious or non-infectious origin, nervous system diseases of infectious or non-infectious origin, muscle diseases of infectious or non-infectious origin, bone diseases of infectious or non-infectious origin, reproductive system diseases of infectious or non-infectious origin, renal system diseases of infectious or non-infectious origin, blood diseases of infectious or non-infectious origin, lymphatic system diseases of infectious or non-infectious origin, immune system diseases of infectious or non-infectious origin, mental-illness of infectious or non-infectious origin and the like.

In some embodiments, the disease to be treated is a CNS or CNS related disease or disorder, such as a genetic CNS disease or disorder. Such CNS or CNS related disease (including genetic CNS disease or disorders) are described in greater detail elsewhere herein.

Other diseases and disorders will be appreciated by those of skill in the art.

### Adoptive Cell Therapies

Generally speaking, adoptive cell transfer involves the transfer of cells (autologous, allogeneic, and/or xenogeneic) to a subject. The cells may or may not be modified and/or otherwise manipulated prior to delivery to the subject. Manipulation can include genetic modification by one or more gene modifying agents. Exemplary gene modifying agents and systems are described in greater detail elsewhere herein and will be appreciated by those of ordinary skill in the art. Such gene or other modification compositions or systems can be delivered to a cell to be modified for adoptive therapy by one or more of the compositions described herein containing a CNS specific targeting moiety.

In some embodiments, an engineered cell as described herein can be included in an adoptive cell transfer therapy. In some embodiments, an engineered cell as described herein can be delivered to a subject in need thereof. In some embodiments, the cell can be isolated from a subject, manipulated in vitro such that it is capable of generating an engineered AAV capsid particle described herein to produce an engineered cell and delivered back to the subject in an autologous manner or to a different subject in an allogeneic or xenogeneic manner. The cell isolated, manipulated, and/or delivered can be a eukaryotic cell. The cell isolated, manipulated, and/or delivered can be a stem cell. The cell isolated, manipulated, and/or delivered can be a differentiated cell. The cell isolated, manipulated, and/or delivered can be a nervous system cell, such as a central nervous system cell, including but not limited to a neuron, a glial cell, an astrocyte, a Schwann cell, a microglial cell, or other neuron support cell, and/or other brain or CNS cell, or any combination thereof. Other specific cell types will instantly be appreciated by one of ordinary skill in the art.

In some embodiments, the isolated cell can be manipulated such that it becomes an engineered cell as described elsewhere herein (e.g., contain and/or express one or more engineered delivery system molecules or vectors described elsewhere herein). Methods of making such engineered cells are described in greater detail elsewhere herein.

### Gene Drives

The present invention also contemplates use of the engineered delivery system molecules, vectors, engineered cells, and/or engineered AAV capsid particles described herein to generate a gene drive via delivery of one or more cargo polynucleotides or production of engineered AAV capsid particles with one or more cargo polynucleotides capable of producing a gene drive. In some embodiments, the gene drive can be a Cas-mediated RNA-guided gene drive e.g., Cas- to provide RNA-guided gene drives, for example in systems analogous to gene drives described in PCT Patent Publication WO 2015/105928. Systems of this kind may for example provide methods for altering eukaryotic germline cells, by introducing into the germline cell a nucleic acid sequence encoding an RNA-guided DNA nuclease and one or more guide RNAs. The guide RNAs may be designed to be complementary to one or more target locations on genomic DNA of the germline cell. The nucleic acid sequence encoding the RNA guided DNA nuclease and the nucleic acid sequence encoding the guide RNAs may be provided on constructs between flanking sequences, with promoters arranged such that the germline cell may express the RNA guided DNA nuclease and the guide RNAs, together with any desired cargo-encoding sequences that are also situated between the flanking sequences. The flanking sequences will typically include a sequence which is identical to a corresponding sequence on a selected target chromosome, so that the flanking sequences work with the components encoded by the construct to facilitate insertion of the foreign nucleic acid construct sequences into genomic DNA at a target cut site by mechanisms such as homologous recombination, to render the germline cell homozygous for the foreign nucleic acid sequence. In this way, gene-drive systems are capable of introgressing desired cargo genes throughout a breeding population (Gantz et al., 2015, Highly efficient Cas9-mediated gene drive for population modification of the malaria vector mosquito Anopheles stephensi, PNAS 2015, published ahead of print Nov. 23, 2015, doi:10.1073/pnas.1521077112; Esvelt et al., 2014, Concerning RNA-guided gene drives for the alteration of wild populations eLife 2014; 3:e03401). In select embodiments, target sequences may be selected which have few potential off-target sites in a genome. Targeting multiple sites within a target locus, using multiple guide RNAs, may increase the cutting frequency and hinder the evolution of drive resistant alleles. Truncated guide RNAs may reduce off-target cutting. Paired nickases may be used instead of a single nuclease, to further increase specificity. Gene drive constructs (such as gene drive engineered delivery system constructs) may include cargo sequences encoding transcriptional regulators, for example to activate homologous recombination genes and/or repress non-homologous end-joining. Target sites may be chosen within an essential gene, so that non-homologous end-joining events may cause lethality rather than creating a drive-resistant allele. The gene drive constructs can be engineered to function in a range of hosts at a range of temperatures (Cho et al. 2013, Rapid and Tunable Control of Protein Stability in Caenorhabditis elegans Using a Small Molecule, PLoS ONE 8(8): e72393. doi:10.1371/journal.pone.0072393).

### Transplantation and Xenotransplantation

The engineered AAV capsid system molecules, vectors, engineered cells, and/or engineered delivery particles described herein, can be used to deliver cargo polynucleotides and/or otherwise be involved in modifying tissues for transplantation between two different persons (transplantation) or between species (xenotransplantation). Such techniques for generation of transgenic animals are described elsewhere herein. Interspecies transplantation techniques are generally known in the art. For example, RNA-guided DNA nucleases can be delivered using via engineered AAV capsid polynucleotides, vectors, engineered cells, and/or engineered AAV capsid particles described herein and can be used to knockout, knockdown or disrupt selected genes in an organ for transplant (e.g., ex vivo (e.g., after harvest but before transplantation) or in vivo (in donor or recipient)), animal, such as a transgenic pig (such as the human heme oxygenase-1 transgenic pig line), for example by disrupting expression of genes that encode epitopes recognized by the human immune system, i.e., xenoantigen genes. Candidate porcine genes for disruption may for example include α(1,3)-galactosyltransferase and cytidine monophosphate-N-acetylneuraminic acid hydroxylase genes (see PCT Patent Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

Where it is interspecies transplantation (such as human to human), the engineered AAV capsid system molecules, vectors, engineered cells, and/or engineered delivery particles described herein, can be used to deliver cargo polynucleotides and/or otherwise be involved to modify the tissue to be transplanted. In some embodiments, the modification can include modifying one or more HLA antigens or other tissue type determinants, such that the immunogenic profile is more similar or identical to the recipient's immunogenic profile than to the donor's so as to reduce the occurrence of rejection by the recipient. Relevant tissue type determinants are known in the art (such as those used to determine organ matching) and techniques to determine the immunogenic profile (which is made up of the expression signature of the tissue type determinants) are generally known in the art.

In some embodiments, the donor (such as before harvest) or recipient (after transplantation) can receive one or more of the engineered AAV capsid system molecules, vectors, engineered cells, and/or engineered delivery particles described herein that are capable of modifying the immunogenic profile of the transplanted cells, tissue, and/or organ. In some embodiments, the transplanted cells, tissue, and/or organ can be harvested from the donor and the engineered AAV capsid system molecules, vectors, engineered cells, and/or engineered delivery particles described herein capable of modifying the harvested cells, tissue, and/or organ to be, for example, less immunogenic or be modified to have some specific characteristic when transplanted in the recipient can be delivered to the harvested cells, tissue, and/or organ ex vivo. After delivery the cells, tissue, and/or organs can be transplanted into the donor.

Gene Modification and Treatment of Diseases with Genetic or Epigenetic Embodiments that Affect the CNS, Brain, and/or Neurons, the Eye and/or Inner Ear

The engineered delivery system molecules, vectors, engineered cells, and/or engineered delivery particles described herein (e.g., those with one or more targeting moieties, such as a CNS-specific targeting moiety described herein) can be used to modify genes or other polynucleotides and/or treat diseases of the CNS, brain, and/or neurons, the eye, and/or the inner ear with genetic and/or epigenetic embodiments. As described elsewhere herein the cargo molecule can be a polynucleotide that can be delivered to a cell and, in some embodiments, be integrated into the genome of the cell. In some embodiments, the cargo molecule(s) can be one or more CRISPR-Cas system components. In some embodiments, the CRISPR-Cas components, when delivered by an engineered AAV capsid particles described herein can be optionally expressed in the recipient cell and act to modify the genome of the recipient cell in a sequence specific manner. In some embodiments, the cargo molecules that can be packaged and delivered by the engineered AAV capsid particles described herein can facilitate/mediate genome modification via a method that is not dependent on CRISPR-Cas. Such non-CRISPR-Cas genome modification systems will instantly be appreciated by those of ordinary skill in the art and are also, at least in part, described elsewhere herein. In some embodiments, modification is at a specific target sequence. In other embodiments, modification is at locations that appear to be random throughout the genome.

Exemplary CNS, Brain, and/or Neuronal Disease-Associated Genes

Examples of CNS, brain, and/or neuronal disease-associated genes and polynucleotides that can be modified using the engineered delivery AAV delivery system molecules, vectors, capsids, engineered cells, and/or engineered delivery particles described herein are described below.

In some embodiments, a therapeutic or preventive, such as the engineered AAV capsids and systems thereof as described elsewhere herein, can be delivered to a subject in need thereof or a cell thereof to treat a brain, neuron, neurological, and/or central nervous system disease or disorder (CNS). In some embodiments the brain, neuron, neurological, and/or CNS disease or disorder can be caused, directly or indirectly, by one or mutations in one or more of the following genes as compared to normal or non-pathological variant of the same: in the case of Amyotrophic lateral sclerosis (ALS): SOD1, ALS2, STEX, FUS, TARDBP, VEGF (VEGF-a, VEGF-b, VEGF-c); in the case of Alzheimer's disease: E1, CHIP, UCH, UBB, Tau, LRP, PICALM, Clusterin, PS1, SORL1, CR1, Vldlr, Uba1, Uba3, CHIP28, Aqp1, Uchl1, Uchl3, APP, AAA, CVAP, AD1, APOE, AD2, PSEN2, AD4, STM2, APBB2, FE65L1, NOS3, PLAU, URK, ACE, DCP1, ACE1, MPO, PACIP1, PAXIP1L, PTIP, A2M, BLMH, BMH, PSEN1, AD3); in the case of Autism: Mecp2, BZRAP1, MDGA2, Sema5A, Neurexin 1, GLO1, MECP2, RTT, PPMX, MRX16, MRX79, NLGN3, NLGN4, KIAA1260, AUTSX2; in the case of Fragile X Syndrome: FMR2, FXR1, FXR2, mGLUR5; in the case of Huntington's disease and disease like disorders: HD, IT15, PRNP, PRIP, JPH3, JP3, HDL2, TBP, SCA17); in the case of Parkinson's disease: NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, NDUFV2, PINK1, x-synuclein); in the case of Rett syndrome: MECP2, RTT, PPMX, MRX16, MRX79, CDKL5, STK9, MECP2, RTT, PPMX, MRX16, MRX79, x-Synuclein, DJ-1; in the case of Schizophrenia: Neuregulin1 (Nrg1), Erb4 (receptor for Neuregulin), Complexin1 (Cplx1), Tph1 Tryptophan hydroxylase, Tph2, Tryptophan hydroxylase 2, Neurexin 1, GSK3, GSK3a, GSK3b, 5-HTT (Slc6a4), COMT, DRD (Drd1a), SLC6A3, DAOA, DTNBP1, Dao (Dao1)); in the case of Secretase Related Disorders (APH-1 (alpha and beta), Presenilin (Psen1), nicastrin, (Ncstn), PEN-2, Nos1, Parp1, Nat1, Nat2); in the case of Trinucleotide Repeat Disorders (HTT (Huntington's Dx), SBMA/SMAX1/AR (Kennedy's Dx), FXN/X25 (Friedrich's Ataxia), ATX3 (Machado-Joseph's Dx), ATXN1 and ATXN2 (spinocerebellar ataxias), DMPK (myotonic dystrophy), Atrophin-1 and Atn1 (DRPLA Dx), CBP (Creb-BP—global instability), VLDLR (Alzheimer's), Atxn7, Atxn10); in the case of diseases or disorders associated with or involving aberrant or abnormal axonal guidance signaling in the brain, neurons, and/or CNS: PRKCE; ITGAM; ROCK1; ITGA5; CXCR4; ADAM12; IGF1; RAC1; RAP1A; EIF4E; PRKCZ; NRP1; NTRK2; ARHGEF7; SMO; ROCK2; MAPK1; PGF; RAC2; PTPN11; GNAS; AKT2; PIK3CA; ERBB2; PRKCI; PTK2; CFL1; GNAQ; PIK3CB; CXCL12; PIK3C3; WNT11; PRKD1; GNB2L1; ABL1; MAPK3; ITGA1; KRAS; RHOA; PRKCD; PIK3C2A; ITGB7; GLI2; PXN; VASP; RAF1; FYN; ITGB1; MAP2K2; PAK4; ADAM17; AKT1; PIK3R1; GLI1; WNT5A; ADAM10; MAP2K1; PAK3; ITGB3; CDC42; VEGFA; ITGA2; EPHA8; CRKL; RND1; GSK3B; AKT3; PRKCA; in the case of diseases or disorders associated with or involving aberrant or abnormal actin cytoskeleton signaling in the brain, neurons, and/or CNS: ACTN4; PRKCE; ITGAM; ROCK1; ITGA5; IRAK1; PRKAA2; EIF2AK2; RAC1; INS; ARHGEF7; GRK6; ROCK2; MAPK1; RAC2; PLK1; AKT2; PIK3CA; CDK8; PTK2; CFL1; PIK3CB; MYH9; DIAPH1; PIK3C3; MAPK8; F2R; MAPK3; SLC9A1; ITGA1; KRAS; RHOA; PRKCD; PRKAA1; MAPK9; CDK2; PIM1; PIK3C2A; ITGB7; PPP1CC; PXN; VIL2; RAF1; GSN; DYRK1A; ITGB1; MAP2K2; PAK4; PIP5K1A; PIK3R1; MAP2K1; PAK3; ITGB3; CDC42; APC; ITGA2; TTK; CSNK1A1; CRKL; BRAF; VAV3; SGK; in the case of diseases or disorders associated with or involving Huntington's Disease signaling: PRKCE; IGF1; EP300; RCOR1; PRKCZ; HDAC4; TGM2; MAPK1; CAPNS1; AKT2; EGFR; NCOR2; SP1; CAPN2; PIK3CA; HDAC5; CREB1; PRKCI; HSPA5; REST; GNAQ; PIK3CB; PIK3C3; MAPK8; IGF1R; PRKD1; GNB2L1; BCL2L1; CAPN1; MAPK3; CASP8; HDAC2; HDAC7A; PRKCD; HDAC11; MAPK9; HDAC9; PIK3C2A; HDAC3; TP53; CASP9; CREBBP; AKT1; PIK3R1; PDPK1; CASP1; APAF1; FRAP1; CASP2; JUN; BAX; ATF4; AKT3; PRKCA; CLTC; SGK; HDAC6; CASP3; in the case of diseases or disorders associated with or involving aberrant, pathologic, and/or abnormal apoptosis regulation and/or signaling in the brain, neurons, and/or CNS and/or diseases or disorders thereof: PRKCE; ROCK1; BID; IRAK1; PRKAA2; EIF2AK2; BAK1; BIRC4; GRK6; MAPK1; CAPNS1; PLK1; AKT2; IKBKB; CAPN2; CDK8; FAS; NFKB2; BCL2; MAP3K14; MAPK8; BCL2L1; CAPN1; MAPK3; CASP8; KRAS; RELA; PRKCD; PRKAA1; MAPK9; CDK2; PIM1; TP53; TNF; RAF1; IKBKG; RELB; CASP9; DYRK1A; MAP2K2; CHUK; APAF1; MAP2K1; NFKB1; PAK3; LMNA; CASP2; BIRC2; TTK; CSNK1A1; BRAF; BAX; PRKCA; SGK; CASP3; BIRC3; PARP1; in the case of diseases or disorders associated with or involving aberrant, pathologic, and/or abnormal leukocyte extravasation signaling in the brain, neurons, and/or CNS and/or diseases or disorders thereof: ACTN4; CD44; PRKCE; ITGAM; ROCK1; CXCR4; CYBA; RAC1; RAP1A; PRKCZ; ROCK2; RAC2; PTPN11; MMP14; PIK3CA; PRKCI; PTK2; PIK3CB; CXCL12; PIK3C3; MAPK8; PRKD1; ABL1; MAPK10; CYBB; MAPK13; RHOA; PRKCD; MAPK9; SRC; PIK3C2A; BTK; MAPK14; NOX1; PXN; VIL2; VASP; ITGB1; MAP2K2; CTNND1; PIK3R1; CTNNB1; CLDN1; CDC42; F11R; ITK; CRKL; VAV3; CTTN; PRKCA; MMP1; MMP9; in the case of diseases or disorders associated with or involving aberrant, pathologic, and/or abnormal integrin signaling in the brain, neurons, and/or CNS and/or diseases or disorders thereof: ACTN4; ITGAM; ROCK1; ITGA5; RAC1; PTEN; RAP1A; TLN1; ARHGEF7; MAPK1; RAC2; CAPNS1; AKT2; CAPN2; PIK3CA; PTK2; PIK3CB; PIK3C3; MAPK8; CAV1; CAPN1; ABL1; MAPK3; ITGA1; KRAS; RHOA; SRC; PIK3C2A; ITGB7; PPP1CC; ILK; PXN; VASP; RAF1; FYN; ITGB1; MAP2K2; PAK4; AKT1; PIK3R1; TNK2; MAP2K1; PAK3; ITGB3; CDC42; RND3; ITGA2; CRKL; BRAF; GSK3B; AKT3; in the case of diseases or disorders associated with or involving aberrant, pathologic, and/or abnormal acute phase response signaling in the brain, neurons, and/or CNS and/or diseases or disorders thereof: IRAK1; SOD2; MYD88; TRAF6; ELK1; MAPK1; PTPN11; AKT2; IKBKB; PIK3CA; FOS; NFKB2; MAP3K14; PIK3CB; MAPK8; RIPK1; MAPK3; IL6ST; KRAS; MAPK13; IL6R; RELA; SOCS1; MAPK9; FTL; NR3C1; TRAF2; SERPINE1; MAPK14; TNF; RAF1; PDK1; IKBKG; RELB; MAP3K7; MAP2K2; AKT1; JAK2; PIK3R1; CHUK; STAT3; MAP2K1; NFKB1; FRAP1; CEBPB; JUN; AKT3; ILIR1; IL6; in the case of diseases or disorders associated with or involving aberrant, pathologic, and/or abnormal PTEN signaling in the brain, neurons, and/or CNS and/or diseases or disorders thereof: ITGAM; ITGA5; RAC1; PTEN; PRKCZ; BCL2L11; MAPK1; RAC2; AKT2; EGFR; IKBKB; CBL; PIK3CA; CDKN1B; PTK2; NFKB2; BCL2; PIK3CB; BCL2L1; MAPK3; ITGA1; KRAS; ITGB7; ILK; PDGFRB; INSR; RAF1; IKBKG; CASP9; CDKN1A; ITGB1; MAP2K2; AKT1; PIK3R1; CHUK; PDGFRA; PDPK1; MAP2K1; NFKB1; ITGB3; CDC42; CCND1; GSK3A; ITGA2; GSK3B; AKT3; FOXO1; CASP3; RPS6KB1; in the case of diseases or disorders associated with or involving aberrant, pathologic, and/or abnormal p53 signaling in the brain, neurons, and/or CNS and/or diseases or disorders thereof: PTEN; EP300; BBC3; PCAF; FASN; BRCA1; GADD45A; BIRC5; AKT2; PIK3CA; CHEK1; TP53INP1; BCL2; PIK3CB; PIK3C3; MAPK8; THBS1; ATR; BCL2L1; E2F1; PMAIP1; CHEK2; TNFRSF10B; TP73; RB1; HDAC9; CDK2; PIK3C2A; MAPK14; TP53; LRDD; CDKN1A; HIPK2; AKT1; PIK3R1; RRM2B; APAF1; CTNNB1; SIRT1; CCND1; PRKDC; ATM; SFN; CDKN2A; JUN; SNAI2; GSK3B; BAX; AKT3; in the case of diseases or disorders associated with or involving aberrant, pathologic, and/or abnormal aryl hydrocarbon receptor signaling in the brain, neurons, and/or CNS and/or diseases or disorders thereof: HSPB1; EP300; FASN; TGM2; RXRA; MAPK1; NQO1; NCOR2; SP1; ARNT; CDKN1B; FOS; CHEK1; SMARCA4; NFKB2; MAPK8; ALDH1A1; ATR; E2F1; MAPK3; NRIP1; CHEK2; RELA; TP73; GSTP1; RB1; SRC; CDK2; AHR; NFE2L2; NCOA3; TP53; TNF; CDKN1A; NCOA2; APAF1; NFKB1; CCND1; ATM; ESR1; CDKN2A; MYC; JUN; ESR2; BAX; IL6; CYP1B1; HSP90AA1; in the case of diseases or disorders associated with or involving aberrant, pathologic, and/or abnormal xenobiotic metabolism signaling in the brain, neurons, and/or CNS and/or diseases or disorders thereof: PRKCE; EP300; PRKCZ; RXRA; MAPK1; NQO1; NCOR2; PIK3CA; ARNT; PRKCI; NFKB2; CAMK2A; PIK3CB; PPP2R1A; PIK3C3; MAPK8; PRKD1; ALDH1AI; MAPK3; NRIP1; KRAS; MAPK13; PRKCD; GSTP1; MAPK9; NOS2A; ABCB1; AHR; PPP2CA; FTL; NFE2L2; PIK3C2A; PPARGC1A; MAPK14; TNF; RAF1; CREBBP; MAP2K2; PIK3R1; PPP2R5C; MAP2K1; NFKB1; KEAP1; PRKCA; EIF2AK3; IL6; CYP1B1; HSP90AA1; in the case of diseases or disorders associated with or involving aberrant, pathologic, and/or abnormal SAPK/JNK signaling in the brain, neurons, and/or CNS and/or diseases or disorders thereof: PRKCE; IRAK1; PRKAA2; EIF2AK2; RAC1; ELK1; GRK6; MAPK1; GADD45A; RAC2; PLK1; AKT2; PIK3CA; FADD; CDK8; PIK3CB; PIK3C3; MAPK8; RIPK1; GNB2L1; IRS1; MAPK3; MAPK10; DAXX; KRAS; PRKCD; PRKAA1; MAPK9; CDK2; PIM1; PIK3C2A; TRAF2; TP53; LCK; MAP3K7; DYRK1A; MAP2K2; PIK3R1; MAP2K1; PAK3; CDC42; JUN; TTK; CSNK1A1; CRKL; BRAF; SGK; in the case of diseases or disorders associated with or involving aberrant, pathologic, and/or abnormal PPAr/RXR signaling in the brain, neurons, and/or CNS and/or diseases or disorders thereof: PRKAA2; EP300; INS; SMAD2; TRAF6; PPARA; FASN; RXRA; MAPK1; SMAD3; GNAS; IKBKB; NCOR2; ABCA1; GNAQ; NFKB2; MAP3K14; STAT5B; MAPK8; IRS1; MAPK3; KRAS; RELA; PRKAA1; PPARGC1A; NCOA3; MAPK14; INSR; RAF1; IKBKG; RELB; MAP3K7; CREBBP; MAP2K2; JAK2; CHUK; MAP2K1; NFKB1; TGFBR1; SMAD4; JUN; IL1R1; PRKCA; IL6; HSP90AA1; ADIPOQ; in the case of diseases or disorders associated with or involving aberrant, pathologic, and/or abnormal NF-kappaB signaling in the brain, neurons, and/or CNS and/or diseases or disorders thereof: IRAK1; EIF2AK2; EP300; INS; MYD88; PRKCZ; TRAF6; TBK1; AKT2; EGFR; IKBKB; PIK3CA; BTRC; NFKB2; MAP3K14; PIK3CB; PIK3C3; MAPK8; RIPK1; HDAC2; KRAS; RELA; PIK3C2A; TRAF2; TLR4; PDGFRB; TNF; INSR; LCK; IKBKG; RELB; MAP3K7; CREBBP; AKT1; PIK3R1; CHUK; PDGFRA; NFKB1; TLR2; BCL10; GSK3B; AKT3; TNFAIP3; IL1R1; in the case of diseases or disorders associated with or involving aberrant, pathologic, and/or abnormal neuregulin signaling in the brain, neurons, and/or CNS and/or diseases or disorders thereof: ERBB4; PRKCE; ITGAM; ITGA5; PTEN; PRKCZ; ELK1; MAPK1; PTPN11; AKT2; EGFR; ERBB2; PRKCI; CDKN1B; STAT5B; PRKD1; MAPK3; ITGA1; KRAS; PRKCD; STAT5A; SRC; ITGB7; RAF1; ITGB1; MAP2K2; ADAM17; AKT1; PIK3R1; PDPK1; MAP2K1; ITGB3; EREG; FRAP1; PSEN1; ITGA2; MYC; NRG1; CRKL; AKT3; PRKCA; HSP90AA1; RPS6KB1; in the case of diseases or disorders associated with or involving aberrant, pathologic, and/or abnormal wnt and beta catenin signaling in the brain, neurons, and/or CNS and/or diseases or disorders thereof: CD44; EP300; LRP6; DVL3; CSNK1E; GJA1; SMO; AKT2; PIN1; CDH1; BTRC; GNAQ; MARK2; PPP2R1A; WNT11; SRC; DKK1; PPP2CA; SOX6; SFRP2; ILK; LEF1; SOX9; TP53; MAP3K7; CREBBP; TCF7L2; AKT1; PPP2R5C; WNT5A; LRP5; CTNNB1; TGFBR1; CCND1; GSK3A; DVL1; APC; CDKN2A; MYC; CSNK1A1; GSK3B; AKT3; SOX2; in the case of diseases or disorders associated with or involving aberrant, pathologic, and/or abnormal insulin receptor signaling in the brain, neurons, and/or CNS and/or diseases or disorders thereof: PTEN; INS; EIF4E; PTPN1; PRKCZ; MAPK1; TSC1; PTPN11; AKT2; CBL; PIK3CA; PRKCI; PIK3CB; PIK3C3; MAPK8; IRS1; MAPK3; TSC2; KRAS; EIF4EBP1; SLC2A4; PIK3C2A; PPP1CC; INSR; RAF1; FYN; MAP2K2; JAK1; AKT1; JAK2; PIK3R1; PDPK1; MAP2K1; GSK3A; FRAP1; CRKL; GSK3B; AKT3; FOXO1; SGK; RPS6KB1; in the case of diseases or disorders associated with or involving aberrant, pathologic, and/or abnormal IL-6 signaling in the brain, neurons, and/or CNS and/or diseases or disorders thereof: HSPB1; TRAF6; MAPKAPK2; ELK1; MAPK1; PTPN11; IKBKB; FOS; NFKB2; MAP3K14; MAPK8; MAPK3; MAPK10; IL6ST; KRAS; MAPK13; IL6R; RELA; SOCS1; MAPK9; ABCB1; TRAF2; MAPK14; TNF; RAF1; IKBKG; RELB; MAP3K7; MAP2K2; IL8; JAK2; CHUK; STAT3; MAP2K1; NFKB1; CEBPB; JUN; ILIR1; SRF; IL6; in the case of diseases or disorders associated with or involving aberrant, pathologic, and/or abnormal IGF-1 signaling in the brain, neurons, and/or CNS and/or diseases or disorders thereof: IGF1; PRKCZ; ELK1; MAPK1; PTPN11; NEDD4; AKT2; PIK3CA; PRKCI; PTK2; FOS; PIK3CB; PIK3C3; MAPK8; IGF1R; IRS1; MAPK3; IGFBP7; KRAS; PIK3C2A; YWHAZ; PXN; RAF1; CASP9; MAP2K2; AKT1; PIK3R1; PDPK1; MAP2K1; IGFBP2; SFN; JUN; CYR61; AKT3; FOXO1; SRF; CTGF; RPS6KB1; in the case of diseases or disorders associated with or involving aberrant, pathologic, and/or abnormal NRF2-mediated oxidative stress response pathway regulation or signaling in the brain, neurons, and/or CNS and/or diseases or disorders thereof: PRKCE; EP300; SOD2; PRKCZ; MAPK1; SQSTM1; NQO1; PIK3CA; PRKCI; FOS; PIK3CB; PIK3C3; MAPK8; PRKD1; MAPK3; KRAS; PRKCD; GSTP1; MAPK9; FTL; NFE2L2; PIK3C2A; MAPK14; RAF1; MAP3K7; CREBBP; MAP2K2; AKT1; PIK3R1; MAP2K1; PP1B; JUN; KEAP1; GSK3B; ATF4; PRKCA; EIF2AK3; HSP90AA1; PRDX1; in the case of diseases or disorders associated with or involving aberrant, pathologic, and/or abnormal PPAR (e.g. PPAR alpha, PPAR beta, PPAR delta, and/or PPAR gamma) regulation or signaling in the brain, neurons, and/or CNS and/or diseases or disorders thereof: EP300; INS; TRAF6; PPARA; RXRA; MAPK1; IKBKB; NCOR2; FOS; NFKB2; MAP3K14; STAT5B; MAPK3; NRIP1; KRAS; PPARG; RELA; STAT5A; TRAF2; PPARGC1A; PDGFRB; TNF; INSR; RAF1; IKBKG; RELB; MAP3K7; CREBBP; MAP2K2; CHUK; PDGFRA; MAP2K1; NFKB1; JUN; ILIR1; HSP90AA1; in the case of diseases or disorders associated with or involving aberrant, pathologic, and/or abnormal Fc Epsilon RI regulation or signaling in the brain, neurons, and/or CNS and/or diseases or disorders thereof: PRKCE; RAC1; PRKCZ; LYN; MAPK1; RAC2; PTPN11; AKT2; PIK3CA; SYK; PRKCI; PIK3CB; PIK3C3; MAPK8; PRKD1; MAPK3; MAPK10; KRAS; MAPK13; PRKCD; MAPK9; PIK3C2A; BTK; MAPK14; TNF; RAF1; FYN; MAP2K2; AKT1; PIK3R1; PDPK1; MAP2K1; AKT3; VAV3; PRKCA; in the case of diseases or disorders associated with or involving aberrant, pathologic, and/or abnormal G-protein coupled receptor regulation or signaling in the brain, neurons, and/or CNS and/or diseases or disorders thereof: PRKCE; RAP1A; RGS16; MAPK1; GNAS; AKT2; IKBKB; PIK3CA; CREB1; GNAQ; NFKB2; CAMK2A; PIK3CB; PIK3C3; MAPK3; KRAS; RELA; SRC; PIK3C2A; RAF1; IKBKG; RELB; FYN; MAP2K2; AKT1; PIK3R1; CHUK; PDPK1; STAT3; MAP2K1; NFKB1; BRAF; ATF4; AKT3; PRKCA; in the case of diseases or disorders associated with or involving aberrant, pathologic, and/or abnormal inositol phosphate metabolism regulation or signaling in the brain, neurons, and/or CNS and/or diseases or disorders thereof: PRKCE; IRAK1; PRKAA2; EIF2AK2; PTEN; GRK6; MAPK1; PLK1; AKT2; PIK3CA; CDK8; PIK3CB; PIK3C3; MAPK8; MAPK3; PRKCD; PRKAA1; MAPK9; CDK2; PIM1; PIK3C2A; DYRK1A; MAP2K2; PIP5K1A; PIK3R1; MAP2K1; PAK3; ATM; TTK; CSNK1A1; BRAF; SGK; in the case of diseases or disorders associated with or involving aberrant, pathologic, and/or abnormal PDGF regulation or signaling in the brain, neurons, and/or CNS and/or diseases or disorders thereof: EIF2AK2; ELK1; ABL2; MAPK1; PIK3CA; FOS; PIK3CB; PIK3C3; MAPK8; CAV1; ABL1; MAPK3; KRAS; SRC; PIK3C2A; PDGFRB; RAF1; MAP2K2; JAK1; JAK2; PIK3R1; PDGFRA; STAT3; SPHK1; MAP2K1; MYC; JUN; CRKL; PRKCA; SRF; STAT1; SPHK2; in the case of diseases or disorders associated with involving aberrant, pathologic, and/or abnormal VEGF regulation or signaling in the brain, neurons, and/or CNS and/or diseases or disorders thereof: ACTN4; ROCK1; KDR; FLT1; ROCK2; MAPK1; PGF; AKT2; PIK3CA; ARNT; PTK2; BCL2; PIK3CB; PIK3C3; BCL2L1; MAPK3; KRAS; HIF1A; NOS3; PIK3C2A; PXN; RAF1; MAP2K2; ELAVL1; AKT1; PIK3R1; MAP2K1; SFN; VEGFA; AKT3; FOXO1; PRKCA; in the case of diseases or disorders associated with or involving aberrant, pathologic, and/or abnormal natural killer cell regulation or signaling in the brain, neurons, and/or CNS and/or diseases or disorders thereof: PRKCE; RAC1; PRKCZ; MAPK1; RAC2; PTPN11; KIR2DL3; AKT2; PIK3CA; SYK; PRKCI; PIK3CB; PIK3C3; PRKD1; MAPK3; KRAS; PRKCD; PTPN6; PIK3C2A; LCK; RAF1; FYN; MAP2K2; PAK4; AKT1; PIK3R1; MAP2K1; PAK3; AKT3; VAV3; PRKCA; in the case of diseases or disorders associated with or involving aberrant, pathologic, and/or abnormal cell cycle G1/S checkpoint regulation or signaling in the brain, neurons, and/or CNS and/or diseases or disorders thereof: HDAC4; SMAD3; SUV39H1; HDAC5; CDKN1B; BTRC; ATR; ABL1; E2F1; HDAC2; HDAC7A; RB1; HDAC11; HDAC9; CDK2; E2F2; HDAC3; TP53; CDKN1A; CCND1; E2F4; ATM; RBL2; SMAD4; CDKN2A; MYC; NRG1; GSK3B; RBL1; HDAC6; in the case of diseases or disorders associated with or involving aberrant, pathologic, and/or abnormal T-cell receptor regulation or signaling in the brain, neurons, and/or CNS and/or diseases or disorders thereof: RAC1; ELK1; MAPK1; IKBKB; CBL; PIK3CA; FOS; NFKB2; PIK3CB; PIK3C3; MAPK8; MAPK3; KRAS; RELA; PIK3C2A; BTK; LCK; RAF1; IKBKG; RELB; FYN; MAP2K2; PIK3R1; CHUK; MAP2K1; NFKB1; ITK; BCL10; JUN; VAV3; in the case of diseases or disorders associated with or involving aberrant, pathologic, and/or abnormal death receptor regulation or signaling in the brain, neurons, and/or CNS and/or diseases or disorders thereof: CRADD; HSPB1; BID; BIRC4; TBK1; IKBKB; FADD; FAS; NFKB2; BCL2; MAP3K14; MAPK8; RIPK1; CASP8; DAXX; TNFRSF10B; RELA; TRAF2; TNF; IKBKG; RELB; CASP9; CHUK; APAF1; NFKB1; CASP2; BIRC2; CASP3; BIRC3; in the case of diseases or disorders associated with or involving aberrant, pathologic, and/or FGF regulation or signaling in the brain, neurons, and/or CNS and/or diseases or disorders thereof: RAC1; FGFR1; MET; MAPKAPK2; MAPK1; PTPN11; AKT2; PIK3CA; CREB1; PIK3CB; PIK3C3; MAPK8; MAPK3; MAPK13; PTPN6; PIK3C2A; MAPK14; RAF1; AKT1; PIK3R1; STAT3; MAP2K1; FGFR4; CRKL; ATF4; AK3; PRKCA; HGF; in the case of diseases or disorders associated with or involving aberrant, pathologic, and/or GM-CSF regulation or signaling in the brain, neurons, and/or CNS and/or diseases or disorders thereof: LYN; ELK1; MAPK1; PTPN11; AKT2; PIK3CA; CAMK2A; STAT5B; PIK3CB; PIK3C3; GNB2L1; BCL2L1; MAPK3; ETS1; KRAS; RUNX1; PIM1; PIK3C2A; RAF1; MAP2K2; AKT1; JAK2; PIK3R1; STAT3; MAP2K1; CCND1; AK3; STAT1; in the case of diseases or disorders associated with or involving aberrant, pathologic, and/or amyotrophic lateral sclerosis regulation or signaling in the brain, neurons, and/or CNS and/or diseases or disorders thereof: BID; IGF1; RACI; BIRC4; PGF; CAPNS1; CAPN2; PIK3CA; BCL2; PIK3CB; PIK3C3; BCL2L1; CAPN1; PIK3C2A; TP53; CASP9; PIK3R1; RABSA; CASP1; APAF1; VEGFA; BIRC2; BAX; AKT3; CASP3; BIRC3; in the case of diseases or disorders associated with or involving aberrant, pathologic, and/or JAK/Stat regulation or signaling in the brain, neurons, and/or CNS and/or diseases or disorders thereof: PTPN1; MAPK1; PTPN11; AKT2; PIK3CA; STAT5B; PIK3CB; PIK3C3; MAPK3; KRAS; SOCS1; STAT5A; PTPN6; PIK3C2A; RAF1; CDKN1A; MAP2K2; JAK1; AKT1; JAK2; PIK3R1; STAT3; MAP2K1; FRAP1; AKT3; STAT1; in the case of diseases or disorders associated with or involving aberrant, pathologic, and/or nicotinate and nicotinamide metabolism regulation or signaling in the brain, neurons, and/or CNS and/or diseases or disorders thereof: PRKCE; IRAK1; PRKAA2; EIF2AK2; GRK6; MAPK1; PLK1; AKT2; CDK8; MAPK8; MAPK3; PRKCD; PRKAA1; PBEF1; MAPK9; CDK2; PIM1; DYRK1A; MAP2K2; MAP2K1; PAK3; NT5E; TTK; CSNK1A1; BRAF; SGK; in the case of diseases or disorders associated with or involving aberrant, pathologic, and/or chemokine signaling regulation or signaling in the brain, neurons, and/or CNS and/or diseases or disorders thereof: CXCR4; ROCK2; MAPK1; PTK2; FOS; CFL1; GNAQ; CAMK2A; CXCL12; MAPK8; MAPK3; KRAS; MAPK13; RHOA; CCR3; SRC; PPP1CC; MAPK14; NOX1; RAF1; MAP2K2; MAP2K1; JUN; CCL2; PRKCA; in the case of diseases or disorders associated with or involving aberrant, pathologic, and/or IL-2 signaling regulation or signaling in the brain, neurons, and/or CNS and/or diseases or disorders thereof: ELK1; MAPK1; PTPN11; AKT2; PIK3CA; SYK; FOS; STAT5B; PIK3CB; PIK3C3; MAPK8; MAPK3; KRAS; SOCS1; STAT5A; PIK3C2A; LCK; RAFI; MAP2K2; JAK1; AKT1; PIK3R1; MAP2K1; JUN; AKT3; in the case of diseases or disorders associated with or involving synaptic long term depression in the brain, neurons, and/or CNS and/or diseases or disorders thereof: PRKCE; IGF1; PRKCZ; PRDX6; LYN; MAPK1; GNAS; PRKCI; GNAQ; PPP2R1A; IGF1R; PRKD1; MAPK3; KRAS; GRN; PRKCD; NOS3; NOS2A; PPP2CA; YWHAZ; RAF1; MAP2K2; PPP2R5C; MAP2K1; PRKCA; in the case of diseases or disorders associated with or involving aberrant, pathologic, and/or estrogen receptor regulation or signaling in the brain, neurons, and/or CNS and/or diseases or disorders thereof: TAF4B; EP300; CARM1; PCAF; MAPK1; NCOR2; SMARCA4; MAPK3; NRIP1; KRAS; SRC; NR3C1; HDAC3; PPARGC1A; RBM9; NCOA3; RAF1; CREBBP; MAP2K2; NCOA2; MAP2K1; PRKDC; ESR1; ESR2; in the case of diseases or disorders associated with or involving aberrant, pathologic, and/or protein ubiquitination pathway activity, regulation, and/or signaling in the brain, neurons, and/or CNS and/or diseases or disorders thereof: TRAF6; SMURF1; BIRC4; BRCA1; UCHL1; NEDD4; CBL; UBE2I; BTRC; HSPA5; USP7; USP10; FBXW7; USP9X; STUB1; USP22; B2M; BIRC2; PARK2; USP8; USP1; VHL; HSP90AA1; BIRC3; in the case of diseases or disorders associated with or involving aberrant, pathologic, and/or IL-10 regulation or signaling in the brain, neurons, and/or CNS and/or diseases or disorders thereof: TRAF6; CCR1; ELK1; IKBKB; SP1; FOS; NFKB2; MAP3K14; MAPK8; MAPK13; RELA; MAPK14; TNF; IKBKG; RELB; MAP3K7; JAK1; CHUK; STAT3; NFKB1; JUN; IL1R1; IL6; in the case of diseases or disorders associated with or involving aberrant, pathologic, and/or Vitamin D receptor (VDR) and/or RXR regulation or signaling in the brain, neurons, and/or CNS and/or diseases or disorders thereof: PRKCE; EP300; PRKCZ; RXRA; GADD45A; HES1; NCOR2; SP1; PRKCI; CDKN1B; PRKD1; PRKCD; RUNX2; KLF4; YY1; NCOA3; CDKN1A; NCOA2; SPP1; LRP5; CEBPB; FOXO1; PRKCA; in the case of diseases or disorders associated with or involving aberrant, pathologic, and/or TGF-beta regulation or signaling in the brain, neurons, and/or CNS and/or diseases or disorders thereof: EP300; SMAD2; SMURF1; MAPK1; SMAD3; SMAD1; FOS; MAPK8; MAPK3; KRAS; MAPK9; RUNX2; SERPINE1; RAF1; MAP3K7; CREBBP; MAP2K2; MAP2K1; TGFBR1; SMAD4; JUN; SMAD5; in the case of diseases or disorders associated with or involving aberrant, pathologic, and/or Toll-like Receptor activity, regulation, and/or signaling in the brain, neurons, and/or CNS and/or diseases or disorders thereof: IRAK1; EIF2AK2; MYD88; TRAF6; PPARA; ELK1; IKBKB; FOS; NFKB2; MAP3K14; MAPK8; MAPK13; RELA; TLR4; MAPK14; IKBKG; RELB; MAP3K7; CHUK; NFKB1; TLR2; JUN; in the case of diseases or disorders associated with or involving aberrant, pathologic, and/or p38 MAPK activity, regulation, and/or signaling in the brain, neurons, and/or CNS and/or diseases or disorders thereof: HSPB1; IRAK1; TRAF6; MAPKAPK2; ELK1; FADD; FAS; CREB1; DDIT3; RPS6KA4; DAXX; MAPK13; TRAF2; MAPK14; TNF; MAP3K7; TGFBR1; MYC; ATF4; IL1R1; SRF; STAT1; in the case of diseases or disorders associated with or involving aberrant, pathologic, and/or neurotrophin/TRK activity, regulation, and/or signaling in the brain, neurons, and/or CNS and/or diseases or disorders thereof: NTRK2; MAPK1; PTPN11; PIK3CA; CREB1; FOS; PIK3CB; PIK3C3; MAPK8; MAPK3; KRAS; PIK3C2A; RAF1; MAP2K2; AKT1; PIK3R1; PDPK1; MAP2K1; CDC42; JUN; ATF4; in the case of diseases or disorders associated with or involving aberrant, pathologic, and/or FXR and/or RXR activity, regulation, and/or signaling in the brain, neurons, and/or CNS and/or diseases or disorders thereof: INS; PPARA; FASN; RXRA; AKT2; SDC1; MAPK8; APOB; MAPK10; PPARG; MTTP; MAPK9; PPARGC1A; TNF; CREBBP; AKT1; SREBF1; FGFR4; AKT3; FOXO1; in the case of diseases or disorders associated with or involving aberrant, pathologic, and/or synaptic long term potentiation in the brain, neurons, and/or CNS and/or diseases or disorders thereof: PRKCE; RAP1A; EP300; PRKCZ; MAPK1; CREB1; PRKCI; GNAQ; CAMK2A; PRKD1; MAPK3; KRAS; PRKCD; PPP1CC; RAF1; CREBBP; MAP2K2; MAP2K1; ATF4; PRKCA; in the case of diseases or disorders associated with or involving aberrant, pathologic, and/or calcium regulation and/or signaling in the brain, neurons, and/or CNS and/or diseases or disorders thereof: RAP1A; EP300; HDAC4; MAPK1; HDAC5; CREB1; CAMK2A; MYH9; MAPK3; HDAC2; HDAC7A; HDAC11; HDAC9; HDAC3; CREBBP; CALR; CAMKK2; ATF4; HDAC6; in the case of diseases or disorders associated with or involving aberrant, pathologic, and/or EGF or EGFR regulation and/or signaling in the brain, neurons, and/or CNS and/or diseases or disorders thereof: ELK1; MAPK1; EGFR; PIK3CA; FOS; PIK3CB; PIK3C3; MAPK8; MAPK3; PIK3C2A; RAF1; JAK1; PIK3R1; STAT3; MAP2K1; JUN; PRKCA; SRF; STAT1; in the case of diseases or disorders associated with or involving aberrant, pathologic, and/or LPS/IL-1 mediated inhibition of RXR function, regulation and/or signaling in the brain, neurons, and/or CNS and/or diseases or disorders thereof: IRAK1; MYD88; TRAF6; PPARA; RXRA; ABCA1; MAPK8; ALDH1A1; GSTP1; MAPK9; ABCB1; TRAF2; TLR4; TNF; MAP3K7; NR1H2; SREBF1; JUN; IL1R1; in the case of diseases or disorders associated with or involving aberrant, pathologic, and/or LXR/RXR function, regulation and/or signaling in the brain, neurons, and/or CNS and/or diseases or disorders thereof: FASN; RXRA; NCOR2; ABCA1; NFKB2; IRF3; RELA; NOS2A; TLR4; TNF; RELB; LDLR; NR1H2; NFKB1; SREBF1; IL1R1; CCL2; IL6; MMP9; in the case of diseases or disorders associated with or involving aberrant, pathologic, and/or amyloid processing in the brain, neurons, and/or CNS and/or diseases or disorders thereof: PRKCE; CSNK1E; MAPK1; CAPNS1; AKT2; CAPN2; CAPN1; MAPK3; MAPK13; MAPT; MAPK14; AKT1; PSEN1; CSNK1A1; GSK3B; AKT3; APP; in the case of diseases or disorders associated with or involving aberrant, pathologic, and/or abnormal IL-4 activity, signaling, and/or regulation in the brain, neurons, and/or CNS and/or diseases or disorders thereof: AKT2; PIK3CA; PIK3CB; PIK3C3; IRS1; KRAS; SOCS1; PTPN6; NR3C1; PIK3C2A; JAK1; AKT1; JAK2; PIK3R1; FRAP1; AKT3; RPS6KB1; in the case of diseases or disorders associated with or involving aberrant, pathologic, and/or abnormal cell cycle: G2/M DNA damage checkpoint regulation activity, signaling, and/or regulation in the brain, neurons, and/or CNS and/or diseases or disorders thereof: EP300; PCAF; BRCA1; GADD45A; PLK1; BTRC; CHEK1; ATR; CHEK2; YWHAZ; TP53; CDKN1A; PRKDC; ATM; SFN; CDKN2A; in the case of diseases or disorders associated with or involving aberrant, pathologic, and/or abnormal purine metabolism signaling thereof, and/or regulation thereof in the brain, neurons, and/or CNS and/or diseases or disorders thereof: NME2; SMARCA4; MYH9; RRM2; ADAR; EIF2AK4; PKM2; ENTPD1; RAD51; RRM2B; TJP2; RAD51C; NT5E; POLD1; NME1; in the case of diseases or disorders associated with or involving aberrant, pathologic, and/or abnormal cAMP-mediated signaling and/or regulation in the brain, neurons, and/or CNS and/or diseases or disorders thereof: RAP1A; MAPK1; GNAS; CREB1; CAMK2A; MAPK3; SRC; RAF1; MAP2K2; STAT3; MAP2K1; BRAF; ATF4; in the case of diseases or disorders associated with or involving aberrant, pathologic, and/or abnormal mitochondrial function in the brain, neurons, and/or CNS and/or diseases or disorders thereof: SOD2; MAPK8; CASP8; MAPK10; MAPK9; CASP9; PARK7; PSEN1; PARK2; APP; CASP3; AIF; CytC; SMAC (Diablo); Aifm-1; Aifm-2; in the case of diseases or disorders associated with or involving aberrant, pathologic, and/or abnormal notch signaling and/or regulation in the brain, neurons, and/or CNS and/or diseases or disorders thereof: HES1; JAG1; NUMB; NOTCH4; ADAM17; NOTCH2; PSEN1; NOTCH3; NOTCH1; DLL4; in the case of diseases or disorders associated with or involving aberrant, pathologic, and/or abnormal endoplasmic reticulum stress pathway activity, signaling, and/or regulation in the brain, neurons, and/or CNS and/or diseases or disorders thereof: HSPA5; MAPK8; XBP1; TRAF2; ATF6; CASP9; ATF4; EIF2AK3; CASP3; in the case of diseases or disorders associated with or involving aberrant, pathologic, and/or abnormal pyrimidine metabolism, signaling thereof, and/or regulation thereof in the brain, neurons, and/or CNS and/or diseases or disorders thereof: NME2; AICDA; RRM2; EIF2AK4; ENTPD1; RRM2B; NT5E; POLD1; NME1; in the case of diseases or disorders associated with or involving aberrant, pathologic, and/or abnormal Parkinson's signaling in the brain, neurons, and/or CNS and/or diseases or disorders thereof: UCHL1; MAPK8; MAPK13; MAPK14; CASP9; PARK7; PARK2; CASP3; in the case of diseases or disorders associated with or involving aberrant, pathologic, and/or abnormal Glycolysis/Gluconeogenesis activity, signaling, and/or regulation in the brain, neurons, and/or CNS and/or diseases or disorders thereof: HK2; GCK; GPI; ALDH1A1; PKM2; LDHA; HK1; in the case of diseases or disorders associated with or involving aberrant, pathologic, and/or abnormal interferon activity, signaling, and/or regulation in the brain, neurons, and/or CNS and/or diseases or disorders thereof: IRF1; SOCS1; JAK1; JAK2; IFITM1; STAT1; IFIT3; in the case of diseases or disorders associated with or involving aberrant, pathologic, and/or abnormal sonic the hedgehog activity, signaling, and/or regulation in the brain, neurons, and/or CNS and/or diseases or disorders thereof: ARRB2; SMO; GLI2; DYRK1A; GLI1; GSK3B; DYRK1B; in the case of diseases or disorders associated with or involving aberrant, pathologic, and/or abnormal glycerophospholipid metabolism, signaling thereof, and/or regulation thereof in the brain, neurons, and/or CNS and/or diseases or disorders thereof: PLD1; GRN; GPAM; YWHAZ; SPHK1; SPHK2; in the case of diseases or disorders associated with or involving aberrant, pathologic, and/or abnormal phospholipid degradation, signaling thereof, and/or regulation thereof in the brain, neurons, and/or CNS and/or diseases or disorders thereof: PRDX6; PLD1; GRN; YWHAZ; SPHK1; SPHK2; in the case of diseases or disorders associated with or involving aberrant, pathologic, and/or abnormal tryptophan metabolism, signaling thereof, and/or regulation thereof in the brain, neurons, and/or CNS and/or diseases or disorders thereof: SIAH2; PRMT5; NEDD4; ALDH1A1; CYPIB1; SIAH1; in the case of diseases or disorders associated with or involving aberrant, pathologic, and/or abnormal lysine degradation, signaling thereof, and/or regulation thereof in the brain, neurons, and/or CNS and/or diseases or disorders thereof: SUV39H1; EHMT2; NSD1; SETD7; PPP2R5C; in the case of diseases or disorders associated with or involving aberrant, pathologic, and/or abnormal nucleotide excision repair pathway activity, signaling thereof, and/or regulation thereof in the brain, neurons, and/or CNS and/or diseases or disorders thereof: ERCC5; ERCC4; XPA; XPC; ERCC1; in the case of diseases or disorders associated with or involving aberrant, pathologic, and/or abnormal nucleotide starch and sucrose metabolism, signaling thereof, and/or regulation thereof in the brain, neurons, and/or CNS and/or diseases or disorders thereof: UCHL1; HK2; GCK; GPI; HK1; in the case of diseases or disorders associated with or involving aberrant, pathologic, and/or abnormal aminosugars metabolism, signaling thereof, and/or regulation thereof in the brain, neurons, and/or CNS and/or diseases or disorders thereof: NQO1; HK2; GCK; HK1; in the case of diseases or disorders associated with or involving aberrant, pathologic, and/or abnormal arachidonic acid metabolism, signaling thereof, and/or regulation thereof in the brain, neurons, and/or CNS and/or diseases or disorders thereof: PRDX6; GRN; YWHAZ; CYP1B1; in the case of diseases or disorders associated with or involving aberrant, pathologic, and/or abnormal circadian rhythm signaling and/or regulation in the brain, neurons, and/or CNS and/or diseases or disorders thereof: CSNK1E; CREB1; ATF4; NR1D1; in the case of diseases or disorders associated with or involving aberrant, pathologic, and/or coagulation system activity signaling and/or regulation in the brain, neurons, and/or CNS and/or diseases or disorders thereof: BDKRB1; F2R; SERPINE1; F3; a PAR (e.g. PAR1, PAR2, etc.) PLC, aPC; in the case of diseases or disorders associated with or involving aberrant, pathologic, and/or abnormal dopamine receptor signaling and/or regulation in the brain, neurons, and/or CNS and/or diseases or disorders thereof: PPP2R1A; PPP2CA; PPP1CC; PPP2R5C; in the case of diseases or disorders associated with or involving aberrant, pathologic, and/or abnormal Glutathione Metabolism signaling and/or regulation in the brain, neurons, and/or CNS and/or diseases or disorders thereof: IDH2; GSTP1; ANPEP; IDH1; in the case of diseases or disorders associated with or involving aberrant, pathologic, and/or abnormal Glycerolipid Metabolism signaling and/or regulation in the brain, neurons, and/or CNS and/or diseases or disorders thereof: ALDH1A1; GPAM; SPHK1; SPHK2; in the case of diseases or disorders associated with or involving aberrant, pathologic, and/or abnormal Linoleic Acid Metabolism signaling and/or regulation in the brain, neurons, and/or CNS and/or diseases or disorders thereof: PRDX6; GRN; YWHAZ; CYP1B1; in the case of diseases or disorders associated with or involving aberrant, pathologic, and/or abnormal Methionine Metabolism signaling and/or regulation in the brain, neurons, and/or CNS and/or diseases or disorders thereof: DNMT1; DNMT3B; AHCY; DNMT3A; in the case of diseases or disorders associated with or involving aberrant, pathologic, and/or abnormal Pyruvate Metabolism signaling and/or regulation in the brain, neurons, and/or CNS and/or diseases or disorders thereof: GLO1; ALDH1A1; PKM2; LDHA; in the case of diseases or disorders associated with or involving aberrant, pathologic, and/or abnormal Arginine and Proline Metabolism signaling and/or regulation in the brain, neurons, and/or CNS and/or diseases or disorders thereof: ALDH1A1; NOS3; NOS2A; in the case of diseases or disorders associated with or involving aberrant, pathologic, and/or abnormal Eicosanoid signaling and/or regulation in the brain, neurons, and/or CNS and/or diseases or disorders thereof: PRDX6; GRN; YWHAZ; in the case of diseases or disorders associated with or involving aberrant, pathologic, and/or abnormal fructose and mannose metabolism signaling and/or regulation in the brain, neurons, and/or CNS and/or diseases or disorders thereof: HK2; GCK; HK1; in the case of diseases or disorders associated with or involving aberrant, pathologic, and/or abnormal antigen presentation pathway activity, signaling and/or regulation in the brain, neurons, and/or CNS and/or diseases or disorders thereof: CALR; B2M; in the case of diseases or disorders associated with or involving aberrant, pathologic, and/or abnormal steroid biosynthesis in the brain, neurons, and/or CNS and/or diseases or disorders thereof: NQO1; DHCR7; in the case of diseases or disorders associated with or involving aberrant, pathologic, and/or abnormal butanoate metabolism in the brain, neurons, and/or CNS and/or diseases or disorders thereof: ALDH1A1; NLGN1; in the case of diseases or disorders associated with or involving an aberrant, pathologic, and/or abnormal citrate cycle in the brain, neurons, and/or CNS and/or diseases or disorders thereof: IDH2; IDH1; in the case of diseases or disorders associated with or involving aberrant, pathologic, and/or abnormal fatty acid metabolism in the brain, neurons, and/or CNS and/or diseases or disorders thereof: ALDH1A1; CYP1B1; in the case of diseases or disorders associated with or involving aberrant, pathologic, and/or abnormal Glycerophospholipid metabolism in the brain, neurons, and/or CNS and/or diseases or disorders thereof: PRDX6; CHKA; in the case of diseases or disorders associated with or involving aberrant, pathologic, and/or abnormal histidine metabolism in the brain, neurons, and/or CNS and/or diseases or disorders thereof: PRMT5; ALDH1A1; in the case of diseases or disorders associated with or involving aberrant, pathologic, and/or abnormal inositol metabolism in the brain, neurons, and/or CNS and/or diseases or disorders thereof: ERO1L; APEX1; in the case of diseases or disorders associated with or involving aberrant, pathologic, and/or abnormal Phenylalanine metabolism in the brain, neurons, and/or CNS and/or diseases or disorders thereof: PRDX6; PRDX1; in the case of diseases or disorders associated with or involving aberrant, pathologic, and/or abnormal Seleno amino acid metabolism in the brain, neurons, and/or CNS and/or diseases or disorders thereof: PRMT5; AHCY; in the case of diseases or disorders associated with or involving aberrant, pathologic, and/or abnormal Sphingolipid metabolism in the brain, neurons, and/or CNS and/or diseases or disorders thereof: SPHK1; SPHK2; in the case of diseases or disorders associated with or involving aberrant, pathologic, and/or abnormal Aminophosphonate metabolism in the brain, neurons, and/or CNS and/or diseases or disorders thereof: PRMT5; in the case of diseases or disorders associated with or involving aberrant, pathologic, and/or abnormal androgen and/or estrogen metabolism in the brain, neurons, and/or CNS and/or diseases or disorders thereof: PRMT5; in the case of diseases or disorders associated with or involving aberrant, pathologic, and/or abnormal Ascorbate and Aldarate metabolism in the brain, neurons, and/or CNS and/or diseases or disorders thereof: ALDH1A1; in the case of diseases or disorders associated with or involving aberrant, pathologic, and/or abnormal Cysteine Metabolism in the brain, neurons, and/or CNS and/or diseases or disorders thereof: LDHA; in the case of diseases or disorders associated with or involving aberrant, pathologic, and/or abnormal fatty acid biosynthesis in the brain, neurons, and/or CNS and/or diseases or disorders thereof: FASN; in the case of diseases or disorders associated with or involving aberrant, pathologic, and/or abnormal glutamate receptor signaling in the brain, neurons, and/or CNS and/or diseases or disorders thereof: GNB2L1; in the case of diseases or disorders associated with or involving aberrant, pathologic, and/or abnormal Pentose Phosphate pathway in the brain, neurons, and/or CNS and/or diseases or disorders thereof: GPI; in the case of diseases or disorders associated with or involving aberrant, pathologic, and/or abnormal retinol metabolism in the brain, neurons, and/or CNS and/or diseases or disorders thereof: ALDH1A1; in the case of diseases or disorders associated with or involving aberrant, pathologic, and/or abnormal Pentose and Glucuronate interconversions in the brain, neurons, and/or CNS and/or diseases or disorders thereof: UCHL1; in the case of diseases or disorders associated with or involving aberrant, pathologic, and/or abnormal Riboflavin Metabolism in the brain, neurons, and/or CNS and/or diseases or disorders thereof: TYR; in the case of diseases or disorders associated with or involving aberrant, pathologic, and/or abnormal Tyrosine Metabolism in the brain, neurons, and/or CNS and/or diseases or disorders thereof: PRMT5, TYR; in the case of diseases or disorders associated with or involving aberrant, pathologic, and/or abnormal Ubiquinone biosynthesis in the brain, neurons, and/or CNS and/or diseases or disorders thereof: PRMT5; in the case of diseases or disorders associated with or involving aberrant, pathologic, and/or abnormal Valine, leucine and isoleucine degradation in the brain, neurons, and/or CNS and/or diseases or disorders thereof: ALDH1A1; in the case of diseases or disorders associated with or involving aberrant, pathologic, and/or abnormal glycine, serine, and threonine metabolism in the brain, neurons, and/or CNS and/or diseases or disorders thereof: CHKA; in the case of diseases or disorders associated with or involving aberrant, pathologic, and/or abnormal lysine degradation in the brain, neurons, and/or CNS and/or diseases or disorders thereof: ALDH1A1; in the case of diseases or disorders associated with or involving aberrant, pathologic, and/or abnormal pain or pain signaling or pain signal generation in the brain, neurons, and/or CNS and/or diseases or disorders thereof: TRPM7; TRPC5; TRPC6; TRPC1; Cnr1; cnr2; Grk2; Trpa1; Pomc; Cgrp; Crf; Pka; Era; Nr2b; TRPM5; Prkaca; Prkacb; Prkar1a; Prkar2a; in the case of diseases or disorders associated with or involving aberrant, pathologic, and/or abnormal brain, neuron, and/or CNS development and/or diseases or disorders thereof: BMP-4; Chordin (Chrd); Noggin (Nog); WNT (Wnt2; Wnt2b; Wnt3a; Wnt4; Wnt5a; Wnt6; Wnt7b; Wnt8b; Wnt9a; Wnt9b; Wnt10a; Wnt10b; Wnt16); beta-catenin; Dkk-1; Frizzled related proteins; Otx-2; Gbx2; FGF-8; Reelin; Dab1; unc-86 (Pou4f1 or Bm3a); Numb; Reln; in the case of diseases or disorders associated with or involving prion disorders of or in the brain, neuron, and/or CNS and/or diseases or disorders thereof: Prp; in the case of substance or activity additions involving activities of the brain, neuron, and/or CNS: Prkce (alcohol); Drd2; Drd4; ABAT (alcohol); GRIA2; Grm5; Grin1; Htr1b; Grin2a; Drd3; Pdyn; Gria1 (alcohol); in the case of diseases or disorders associated with or involving PI3K/AKT signaling and/or regulation thereof in the brain, neuron, and/or CNS and/or diseases or disorders thereof: PRKCE; ITGAM; ITGA5; IRAK1; PRKAA2; EIF2AK2; PTEN; EIF4E; PRKCZ; GRK6; MAPK1; TSC1; PLK1; AKT2; IKBKB; PIK3CA; CDK8; CDKN1B; NFKB2; BCL2; PIK3CB; PPP2R1A; MAPK8; BCL2L1; MAPK3; TSC2; ITGA1; KRAS; EIF4EBP1; RELA; PRKCD; NOS3; PRKAA1; MAPK9; CDK2; PPP2CA; PIM1; ITGB7; YWHAZ; ILK; TP53; RAF1; IKBKG; RELB; DYRK1A; CDKN1A; ITGB1; MAP2K2; JAK1; AKT1; JAK2; PIK3R1; CHUK; PDPK1; PPP2R5C; CTNNB1; MAP2K1; NFKB1; PAK3; ITGB3; CCND1; GSK3A; FRAP1; SFN; ITGA2; ITK; CSNK1A1; BRAF; GSK3B; AKT3; FOXO1; SGK; HSP90AA1; RPS6KB1; in the case of diseases or disorders associated with or involving ERK/MAPK signaling and/or regulation thereof in the brain, neuron, and/or CNS and/or diseases or disorders thereof: PRKCE; ITGAM; ITGA5; HSPB1; IRAK1; PRKAA2; EIF2AK2; RAC1; RAP1A; TLN1; EIF4E; ELK1; GRK6; MAPK1; RAC2; PLK1; AKT2; PIK3CA; CDK8; CREB1; PRKCI; PTK2; FOS; RPS6KA4; PIK3CB; PPP2R1A; PIK3C3; MAPK8; MAPK3; ITGA1; ETS1; KRAS; MYCN; EIF4EBP1; PPARG; PRKCD; PRKAA1; MAPK9; SRC; CDK2; PPP2CA; PIM1; PIK3C2A; ITGB7; YWHAZ; PPP1CC; KSR1; PXN; RAF1; FYN; DYRK1A; ITGB1; MAP2K2; PAK4; PIK3R1; STAT3; PPP2R5C; MAP2K1; PAK3; ITGB3; ESR1; ITGA2; MYC; TTK; CSNK1A1; CRKL; BRAF; ATF4; PRKCA; SRF; STAT1; SGK; in the case of diseases or disorders associated with or involving glucocorticoid receptor signaling and/or regulation thereof in the brain, neuron, and/or CNS and/or diseases or disorders thereof: RACI; TAF4B; EP300; SMAD2; TRAF6; PCAF; ELK1; MAPK1; SMAD3; AKT2; IKBKB; NCOR2; UBE2I; PIK3CA; CREB1; FOS; HSPA5; NFKB2; BCL2; MAP3K14; STAT5B; PIK3CB; PIK3C3; MAPK8; BCL2L1; MAPK3; TSC22D3; MAPK10; NRIP1; KRAS; MAPK13; RELA; STAT5A; MAPK9; NOS2A; PBX1; NR3C1; PIK3C2A; CDKN1C; TRAF2; SERPINE1; NCOA3; MAPK14; TNF; RAF1; IKBKG; MAP3K7; CREBBP; CDKN1A; MAP2K2; JAK1; IL8; NCOA2; AKT1; JAK2; PIK3R1; CHUK; STAT3; MAP2K1; NFKB1; TGFBR1; ESR1; SMAD4; CEBPB; JUN; AR; AKT3; CCL2; MMP1; STAT1; IL6; HSP90AA1; in the case of diseases or disorders associated with or involving ephrin receptor signaling and/or regulation thereof in the brain, neuron, and/or CNS and/or diseases or disorders thereof: PRKCE; ITGAM; ROCK1; ITGA5; CXCR4; IRAK1; PRKAA2; EIF2AK2; RAC1; RAP1A; GRK6; ROCK2; MAPK1; PGF; RAC2; PTPN11; GNAS; PLK1; AKT2; DOK1; CDK8; CREB1; PTK2; CFL1; GNAQ; MAP3K14; CXCL12; MAPK8; GNB2L1; ABL1; MAPK3; ITGA1; KRAS; RHOA; PRKCD; PRKAA1; MAPK9; SRC; CDK2; PIM1; ITGB7; PXN; RAF1; FYN; DYRK1A; ITGB1; MAP2K2; PAK4; AKT1; JAK2; STAT3; ADAM10; MAP2K1; PAK3; ITGB3; CDC42; VEGFA; ITGA2; EPHA8; TTK; CSNK1A1; CRKL; BRAF; PTPN13; ATF4; AKT3; SGK; in the case of diseases or disorders associated with or involving B cell receptor signaling and/or regulation thereof in the brain, neuron, and/or CNS and/or diseases or disorders thereof: RAC1; PTEN; LYN; ELK1; MAPK1; RAC2; PTPN11; AKT2; IKBKB; PIK3CA; CREB1; SYK; NFKB2; CAMK2A; MAP3K14; PIK3CB; PIK3C3; MAPK8; BCL2L1; ABL1; MAPK3; ETS1; KRAS; MAPK13; RELA; PTPN6; MAPK9; EGR1; PIK3C2A; BTK; MAPK14; RAF1; IKBKG; RELB; MAP3K7; MAP2K2; AKT1; PIK3R1; CHUK; MAP2K1; NFKB1; CDC42; GSK3A; FRAP1; BCL6; BCL10; JUN; GSK3B; ATF4; AKT3; VAV3; RPS6KB1; in the case of Infantile neuroaxonal dystroph: PLA2G6; in the case of Gaucher's disease: GBA; in the case of Krabbe disease: GALC; in the case of metachromatic leukodystrophy: ARSA and/or PRSP, isoform specific Saposin B replacement; in the case of Salla disease: SLC17A5; in the case of Farber disease or spinal muscular atrophy with progressive myoclonic epilepsy (also referred to as Jankovic-Rivera syndrome): ASAH1; in the case of Unverricht-Lundborg disease: CSTB; in the case of AADC deficiency: AADC; in the case of autosomal recessive forms of Parkinson's disease: PRKN, and others; in the case of Batten disease: CLN3; in the case of giant axonal neuropathy: GAN; in the case of mucopolysacchariodosis diseases (including MOS1H (Hurler syndrome), MPSII (Hunter syndrome), MPS III A-D: IDUA, IDS, SGSH, NAGLU, HGSNAT, GNS; in the case of Sandhoff disease (HEXB); in the case of GM2 gangliosidosis, AB variant: GM2A; in the case of Canavan disease: ASPA; in the case of cockayne syndrome: CSA or CSB; in the case of neurofibromatosis: NF1 or NF2; or any combination thereof.

### Exemplary Eye Diseases and Associated Genes

Examples of eye disease-associated genes and polynucleotides that can be modified using the engineered delivery AAV delivery system molecules, vectors, capsids, engineered cells, and/or engineered delivery particles described herein are described below. The compositions described herein can be delivered to one or both eyes to treat or prevent an eye disease, disorder or symptom thereof.

The compositions described herein can be used to correct ocular defects that arise from several genetic mutations further described in Genetic Diseases of the Eye, Second Edition, edited by Elias I. Traboulsi, Oxford University Press, 2012.

In some embodiments, the condition to be treated or targeted is an eye disorder. In some embodiments, the eye disorder may include glaucoma. In some embodiments, the eye disorder includes a retinal degenerative disease. In some embodiments, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In some embodiments, the retinal degenerative disease is Leber Congenital Amaurosis (LCA) or Retinitis Pigmentosa. Other exemplary eye diseases are described in greater detail elsewhere herein.

In the case of macular degeneration and/or diabetic retinopathy, the gene target can be VEGF, where the gene expression or gene product of VEGF is reduced or eliminated in the eye, particularly the retina, and particularly when applied subretinally or via another ocular administration route.

In the case of Best disease, the gene or gene product target can be RDS or VMD2, where knockdown/reduction or elimination of the gene expression or gene product can provide a therapeutic or otherwise beneficial effect, particularly when applied subretinally or via another ocular administration route.

In the case of Sorsby's fundus dystrophy, the gene or gene product target can be TIMP3, where knockdown/reduction or elimination of the gene expression or gene product can provide a therapeutic or otherwise beneficial effect, particularly when applied subretinally or via another ocular administration route.

In the case of Stargardt disease, the gene or gene product target can be ABCA4, where knockdown/reduction or elimination of the gene expression or gene product can provide a therapeutic or otherwise beneficial effect, particularly when applied subretinally or via another ocular administration route.

In the case of Leber's congenital amaurosis type 2, the gene or gene product target can be RPE65, where knockdown/reduction or elimination of the gene expression or gene product can provide a therapeutic or otherwise beneficial effect, particularly when applied subretinally or via another ocular administration route.

In the case of Choroideremia, the gene or gene product target can be CHM, where knockdown/reduction or elimination of the gene expression or gene product can provide a therapeutic or otherwise beneficial effect, particularly when applied subretinally or via another ocular administration route.

Other exemplary eye diseases and/or disorders and genetic targets for treatment or prevention are shown in the Tables below and in Genes and Genetics in Eye Diseases: A Genomic Medicine Approach for Investigating Hereditary and Inflammatory Ocular Disorders. International Journal of Ophthalmology, 2018 and Inherited Retinal Diseases: Therapeutics, Clinical Trials and End Points—A Review. Clinical & Experimental Ophthalmology, 2021, 49, 270-288, and the Herediary Ocular Disease Database—available at PG-6T disorders.eyes.arizona.edu/for-patients/handout-list.

| Disease | Gene/variant |
|---|---|
| AMD | NOS2A, CFH, CF, C2, C3, CFB, HTRA1/LOC, MMP-9, |
| | TIMP-3, SLC16A8, etc. |
| Cataract | GEMIN4, CYP51A1, RIC1, TAPT1, TAF1A, WDR87, APE1, |
| | MIP, Cx50/GJA3 & 8, CRYAA, CRYBB2, PRX, POLR3B, |
| | XRCC1, ZNF350, EPHA2, etc. |
| Glaucoma | CALM2, MPP-7, Optineurin, LOX1, CYP1B1, CAV1/2, |
| | MYOC, PITX2, FOXC1, PAX6, CYP1B1, LTBP2, etc. |
| Inherited optic neuropathies | Complex I or ND genes, OPA1, RPE65, etc. |
| Marfan syndrome | FBN1, TGFBR2, MTHFR, MTR, MTRR, etc. |
| Myopia | HGF, C-MET, UMODL1, MMP-1/2, PAX6, CBS, MTHFR, |
| | IGF-1, UHRF1BP1L, PTPRR, PPFIA2, P4HA2, etc. |
| Polypoidal choroidal | C2, C3, CFH, SERPING1, PEDF, ARMS2-HTRA1, FGD6, |
| vasculopathies | ABCG1, LOC387715, CETP, etc. |
| Retinitis pigmentosa (RCD) | RPGR, PRPF3, HK1, AGBL5, etc. |
| Stargardt's disease | ABC1, ABCA4, CRB1, etc. |
| Uveal melanoma | PTEN, BAP1, GNAQ, GNA11, DDEF1, SF3B1, EIF1AX, |
| | CDKN2A, p14ARF, HERC2/OCA2, etc. |

| Disease | Gene/variant |
|---|---|
| Best disease | BEST1 |
| X-Linked retinoschisis | RS1 |
| Pattern dystrophy | PRPH2 |
| Sorsby fundus dystrophy | TIMP3 |
| Achromatopsia | CNGB3, CNGA3, GNAT2, ATF6, PDE6H, PDE6C |
| Blu cone monochromatism | OPN1LW, OPN1MW, |
| Bornholm eye disease | OPN1LW, OPN1MW |
| ADGUCA1A-associated | GUCA1A |
| COD/CORD | |
| ADGUCY2D-associated | GUCY2D |
| COD/CORD | |
| Autosomal dominantPRPH2- | PRPH2 |
| associated CORD | |
| Autosomal recessiveABCA4- | ABCA4 |
| associated COD/CORD | |
| X-linkedRPGR- | RPGR |
| associatedCOD/CORD | |
| Fundus Albipunctatus (FA) | RDH5, RLBP1, RPE65 |
| RCD (retinitis pigmentosa) | MERTK, MYO7A, USH2A, PDE6B, RLBP1, RHO, RP2 |
| Enhanced S-conesyndrome | NR2E3 |
| (ESCS) | |
| Bietti crystalline | CYP4V2 |
| corneoretinaldystrophy (BCD) | |
| LEBER | GUCY2D, CEP290, RPE65, AIPL1 |
| CONGENITALAMAUROSIS | |
| (LCA) ANDEARLY-ONSET | |
| SEVERE | |
| RETINALDYSTROPHY | |
| (EOSRD) | |
| Choroideremia (CHM) | CHM |

### Exemplar Inner Diseases and Associated Genes

Examples of ear, particularly inner ear, disease-associated genes and polynucleotides that can be modified using the engineered delivery AAV delivery system molecules, vectors, capsids, engineered cells, and/or engineered delivery particles described herein are described below. The compositions described herein can be delivered to one or both ears, particularly to the inner ear, to treat or prevent an ear disease, disorder or symptom thereof, particularly an inner ear disease, disorder, or symptom thereof.

In certain example embodiments, the inner ear disease or disorder is GJB-2 deafness, Jeryell and Lange-Nielsen syndrome, Usher syndrome, Alport syndrome, Branchio-oto-renal syndrome, Waardenburg syndrome, Pendred syndrome, Stickler syndrome, Treacher Collins syndrome, CHARGE syndrome, Norrie disease, Perrault syndrome, Autosomal dominant Nonsyndromic hearing loss, utosomal Recessive Nonsyndromic Hearing Loss, X-linked nonsyndromic hearing loss, an auditory neuropathy, a congenital hearing loss, or any combination thereof.

In the case of GJB-2 deafness, the GJB-2 gene can be replaced. Genes associated with CHARGE syndrome: SFMA3E, CHD7. Genes associated with Norrie Disease: NDP. Genes associated with Pendred Syndrome: FOMO1, KCNJ10. Genes associated with Perrault syndrome: HSD17B4, HARS2, CLPP*, LARS2, TWNK ERAL1.

Genes associated with Autosomal Dominant Nonsyndromic Hearing Loss may comprise: DIAPH1, KCNQ4, GJB3, IFNLR1, GJB2, GJB6, MYH14, CEACAM16, GSDME/DFNA5, WFS1, LMX1A, TECTA, COCH, EYA4, MYO7A, COL11A2, POU4F3, MYH9, ACTG1, MYO6, SIX1, SLC17A8, REST, GRHL2, NLRP3, TMC1, COL11A1, CRYM, P2RX2, CCDC50, MIRN96, TJP2, TNC, SMAC/DIABLO. TBC1D24, CD164, OSBPL2, HOMER2, KITLG, MCM2, PTPRQ, DMXL2, MYO3A, PDE1C, TRRAP, PLS1, ATP2B2, SCD5, SLC12A2, MAP1B, RIPOR2/FAM65B. Genes associated with Autosomal Recessive Nonsyndromic Hearing Loss may comprise: GJB2, MYO7A, MYO15A, SLC26A4, TMIE, TMC1, TMPRSS3, OTOF, CDH23, GIPC3, STRC, USHIC, OTOG, TECTA, OTOA, PCDH15, RDX, GRXCR1, GAB1, TRIOBP, CLDN14, MYO3A, WHRN, CDC14A, ESRRB, ESPN, MYO6, HGF, ILDR1, ADCY1, CIB2, MARVELD2, BDP1, COL11A2, PDZD7, PJVK, SLC22A4, SLC26A5, LRTOMT/COMT2, DCDC2, LHFPL5, S1PR2, PNPT1, BSND, MSRB3, SYNE4, LOXID1, TPRN, GPSM2, PTPRQ, OTOGL, TBC1D24, ELMOD3, KARS, SERPINB6, CABP2, NARS2, MET, TSPEAR, TMEM132E, PPIP5K2, GRXCR2, EPS8, CLIC5, FAM65B/RIPOR2, EPS8L2, ROR1, WBP2, ESRP1, MPZL2, CEACAM16, GRAP, SPNS2, CLDN9, CLRN2, GAS2. Genes associated X-Linked Nonsyndromic Hearing Loss PRPS1, POU3F4, SMPX, AIFM1, COL4A6. Genes associated with Auditory Neuropathy: DIAPH3.

Other exemplary diseases and associated target gene or gene products for treatment or prevention are shown in the table below and further described in Congenital Hearing Loss. Nature Reviews Disease Primers, 2017, 3.

TABLE 5

| Syndrome | Proteins involved (coding genes) |
|---|---|
| Jervell and | Potassium voltage-gated channel subfamily E member 1 |
| Lange-Nielsen | (KCNE1) and potassium voltage-gated channel subfamily KQT |
| | member 1 (KCNQ1) |
| Usher | Usher syndrome type 1: Unconventional myosin-VIIa |
| | (MYO7A), harmonin (USH1C), cadherin-23 (CDH23), |
| | protocadherin-15 (PCDH15), Usher syndrome type-1G protein |
| | (USH1G) and calcium and integrin-binding family member 2 |
| | (CIB2) |
| | Usher syndrome type 2: usherin (USH2A), adhesion G protein- |
| | coupled receptor V1 (ADGRV1) and whirlin (WHRN) |
| | Usher syndrome type 3: clarin-1 (CLRN1) |
| Alport | Collagen alpha-3(IV) chain (COL4A3), collagen alpha-4(IV) |
| | chain (COL4A4) and collagen alpha-5(IV) chain (COL4A5) |
| Branchio- | Eyes absent homolog 1 (EYA1), homeobox protein SIX1 |
| oto-renal | (SIX1) and homeobox protein SIX5 (SIX5) |
| Waardenburg | Paired box protein Pax-3 (PAX3), microphthalmia-associated |
| | transcription factor (MITF, endothelin-3 (EDN3), endothelin B |
| | receptor (EDNRB), zinc finger protein SNAI2 (SNAI2) and |
| | transcription factor SOX-10 (SOX10) |
| Pendred | Pendrin (SLC26A4) |
| Stickler | Collagen alpha-1151 chain (COL2A1), collagen alpha-1(IX) |
| | chain (COL9A1), collagen alpha-2(IX) chain (COL9A2), |
| | collagen alpha-1(XI) chain (COL11A1) and collagen alpha- |
| | 2(XI) chain (COL11A2) |
| Treacher | Treacle protein (TCOF1), DNA-directed RNA polymerases I |
| Collins | and III subunit RPAC1 (POLR1C) and DNA-directed RNA |
| | polymerases I and III subunit RPAC2 (POLR1D) |

### Method of Modifying Genes

It will be appreciated that in any case where the gene is defective, a gene replacement strategy, gene editing or other approach can be appropriate.

Thus, also described herein are methods of inducing one or more mutations in a eukaryotic or prokaryotic cell (in vitro, i.e., in an isolated eukaryotic cell) as herein discussed comprising delivering to cell a vector as described herein. The mutation(s) can include the introduction, deletion, or substitution of one or more nucleotides at a target sequence of cell(s). In some embodiments, the mutations can include the introduction, deletion, or substitution of 1-75 nucleotides at each target sequence of said cell(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The mutations can include the introduction, deletion, or substitution of 20, 21, 22, 23, 24, 25, 26, 27, 28, 29, 30, 35, 40, 45, 50, or 75 nucleotides at each target sequence of said cell(s). The mutations can include the introduction, deletion, or substitution of 40, 45, 50, 75, 100, 200, 300, 400 or 500 nucleotides at each target sequence of said cell(s). The mutations can include the introduction, deletion, or substitution of 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In some embodiments, the modifications can include the introduction, deletion, or substitution of nucleotides at each target sequence of said cell(s) via nucleic acid components (e.g., guide(s) RNA(s) or sgRNA(s)), such as those mediated by a CRISPR-Cas system.

In some embodiments, the modifications can include the introduction, deletion, or substitution of nucleotides at a target or random sequence of said cell(s) via a non CRISPR-Cas system or technique. Such techniques are discussed elsewhere herein, such as where engineered cells and methods of generating the engineered cells and organisms are discussed.

For minimization of toxicity and off-target effect when using a CRISPR-Cas system, it may be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9-like with the D10A mutation) can be delivered with a pair of guide RNAs targeting a site of interest. Guide sequences and strategies to minimize toxicity and off-target effects can be as in WO 2014/093622 (PCT/US2013/074667); or, via mutation as herein.

Typically, in the context of an endogenous CRISPR system, formation of a CRISPR complex (comprising a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. Without wishing to be bound by theory, a tracr sequence, which may comprise or consist of all or a portion of a wild-type tracr sequence (e.g., about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to a guide sequence.

In one embodiment, the invention provides a method of modifying a target polynucleotide in a eukaryotic cell. In some embodiments, the method includes delivering an engineered targeting moiety, polypeptide, polynucleotide, vector, vector system, particle, viral (e.g., AAV) particle, cell, or any combination thereof described herein having a CRISPR-Cas molecule as a cargo molecule to a subject and/or cell. The CRISPR-Cas system molecule(s) delivered can complex to bind to the target polynucleotide, e.g., to effect cleavage of said target polynucleotide, thereby modifying the target polynucleotide, wherein the CRISPR complex comprises a CRISPR enzyme complexed with a guide sequence hybridized to a target sequence within said target polynucleotide, wherein said guide sequence can be linked to a tracr mate sequence which in turn hybridizes to a tracr sequence. In some embodiments, said cleavage comprises cleaving one or two strands at the location of the target sequence by said CRISPR enzyme. In some embodiments, said cleavage results in decreased transcription of a target gene. In some embodiments, the method further comprises repairing said cleaved target polynucleotide by homologous recombination with an exogenous template polynucleotide, wherein said repair results in a mutation comprising an insertion, deletion, or substitution of one or more nucleotides of said target polynucleotide. In some embodiments, said mutation results in one or more amino acid changes in a protein expressed from a gene comprising the target sequence. In some embodiments, the method further comprises delivering one or more vectors to said eukaryotic cell, wherein one or more vectors comprise the CRISPR enzyme and one or more vectors drive expression of one or more of: the guide sequence linked to the tracr mate sequence, and the tracr sequence. In some embodiments, said CRISPR enzyme drive expression of one or more of: the guide sequence linked to the tracr mate sequence, and the tracr sequence. In some embodiments such CRISPR enzyme are delivered to the eukaryotic cell in a subject. In some embodiments, said modifying takes place in said eukaryotic cell in a cell culture. In some embodiments, the method further comprises isolating said eukaryotic cell from a subject prior to said modifying. In some embodiments, the method further comprises returning said eukaryotic cell and/or cells derived therefrom to said subject. In some embodiments, the isolated cells can be returned to the subject after delivery of one or more engineered targeting moieties, polypeptides, polynucleotides, vectors, vector systems, particles, viral (e.g., AAV) particles, cells, or any combination thereof described herein to the isolated cell. In some embodiments, the isolated cells can be returned to the subject after delivering one or more molecules of the engineered delivery system described herein to the isolated cell, thus making the isolated cells engineered cells as previously described.

### Screening and Cell Selection

The targeting moieties, polypeptides, polynucleotides, vectors, vector systems, particles, viral (e.g., AAV) particles, cells, or any combination thereof described herein described herein can be used in a screening assay and/or cell selection assay. The engineered targeting moieties, polypeptides, polynucleotides, vectors, vector systems, particles, viral (e.g., AAV) particles, cells, or any combination thereof described herein can be delivered to a subject and/or cell. In some embodiments, the cell is a eukaryotic cell. The cell can be in vitro, ex vivo, in situ, or in vivo. The targeting moieties, polypeptides, polynucleotides, vectors, vector systems, particles, viral (e.g., AAV) particles, cells, or any combination thereof described herein can introduce an exogenous molecule or compound, such as a cargo, to subject or cell to which they are delivered. The presence of an exogenous molecule or compound can be detected which can allow for identification of a cell and/or attribute thereof. In some embodiments, the delivered molecules or particles can impart a gene or other nucleotide modification (e.g., mutations, gene or polynucleotide insertion and/or deletion, etc.). In some embodiments the nucleotide modification can be detected in a cell by sequencing. In some embodiments, the nucleotide modification can result in a physiological and/or biological modification to the cell that results in a detectable phenotypic change in the cell, which can allow for detection, identification, and/or selection of the cell. In some embodiments, the phenotypic change can be cell death, such as embodiments where binding of a CRISPR complex to a target polynucleotide results in cell death. Embodiments of the invention allow for selection of specific cells without requiring a selection marker or a two-step process that may include a counter-selection system. The cell(s) may be prokaryotic or eukaryotic cells.

In one embodiment the invention provides for a method of selecting one or more cell(s) by introducing one or more mutations in a gene in the one or more cell (s), the method comprising: introducing one or more vectors, which can include one or more engineered delivery system molecules or vectors described elsewhere herein, into the cell (s), wherein the one or more vectors can include a CRISPR enzyme and/or drive expression of one or more of: a guide sequence linked to a tracr mate sequence, a tracr sequence, and an editing template; or other polynucleotide to be inserted into the cell and/or genome thereof; wherein, for example that which is being expressed is within and expressed in vivo by the CRISPR enzyme and/or the editing template, when included, comprises the one or more mutations that abolish CRISPR enzyme cleavage; allowing homologous recombination of the editing template with the target polynucleotide in the cell(s) to be selected; allowing a CRISPR complex to bind to a target polynucleotide to effect cleavage of the target polynucleotide within said gene, wherein the CRISPR complex comprises the CRISPR enzyme complexed with (1) the guide sequence that is hybridized to the target sequence within the target polynucleotide, and (2) the tracr mate sequence that is hybridized to the tracr sequence, wherein binding of the CRISPR complex to the target polynucleotide induces cell death, thereby allowing one or more cell(s) in which one or more mutations have been introduced to be selected. In a preferred embodiment, the CRISPR enzyme is a Cas protein. In another embodiment of the invention the cell to be selected may be a eukaryotic cell.

The screening methods involving the engineered targeting moieties, polypeptides, polynucleotides, vectors, vector systems, particles, viral (e.g., AAV) particles, cells, or any combination thereof described herein, including but not limited to those that deliver one more CRISPR-Cas system molecules to cell, can be used in detection methods such as fluorescence in situ hybridization (FISH). In some embodiments, one or more components of an engineered CRISPR-Cas system that includes a catalytically inactive Cas protein, can be delivered by engineered targeting moieties, polypeptides, polynucleotides, vectors, vector systems, particles, viral (e.g., AAV) particles, cells, or any combination thereof described herein to a cell and used in a FISH method. The CRISPR-Cas system can include an inactivated Cas protein (dCas) (e.g., a dCas9), which lacks the ability to produce DNA double-strand breaks may be fused with a marker, such as fluorescent protein, such as the enhanced green fluorescent protein (eEGFP) and co-expressed with small guide RNAs to target pericentric, centric and teleomeric repeats in vivo. The dCas system can be used to visualize both repetitive sequences and individual genes in the human genome. Such new applications of labelled dCas, dCas CRISPR-Cas systems, engineered targeting moieties, polypeptides, polynucleotides, vectors, vector systems, particles, viral (e.g., AAV) particles, cells, or any combination thereof described herein can be used in imaging cells and studying the functional nuclear architecture, especially in cases with a small nucleus volume or complex 3-D structures. (Chen B, Gilbert L A, Cimini B A, Schnitzbauer J, Zhang W, Li G W, Park J, Blackbum E H, Weissman J S, Qi L S, Huang B. 2013. Dynamic imaging of genomic loci in living human cells by an optimized CRISPR/Cas system. Cell 155(7):1479-91. doi: 10.1016/j.cell.2013.12.001., the teachings of which can be applied and/or adapted to the CRISPR systems described herein. A similar approach involving a polynucleotide fused to a marker (e.g., a fluorescent marker) can be delivered to a cell via engineered targeting moieties, polypeptides, polynucleotides, vectors, vector systems, particles, viral (e.g., AAV) particles, cells, or any combination thereof described herein and integrated into the genome of the cell and/or otherwise interact with a region of the genome of a cell for FISH analysis.

Similar approaches for studying other cell organelles and other cell structures can be accomplished by delivering to the cell (e.g., via an engineered targeting moieties, polypeptides, polynucleotides, vectors, vector systems, particles, viral (e.g., AAV) particles, cells, or any combination thereof described herein) one or more molecules fused to a marker (such as a fluorescent marker), wherein the molecules fused to the marker are capable of targeting one or more cell structures. By analyzing the presence of the markers, one can identify and/or image specific cell structures.

In some embodiments, the engineered targeting moieties, polypeptides, polynucleotides, vectors, vector systems, particles, viral (e.g., AAV) particles, cells, or any combination thereof described herein can be used in a screening assay inside or outside of a cell. In some embodiments, the screening assay can include delivering a CRISPR-Cas cargo molecule(s) via engineered targeting moieties, polypeptides, polynucleotides, vectors, vector systems, particles, viral (e.g., AAV) particles, cells, or any combination thereof described herein.

Use of the present system in screening is also provided by the present invention, e.g., gain of function screens. Cells which are artificially forced to overexpress a gene are able to down regulate the gene over time (re-establishing equilibrium) e.g., by negative feedback loops. By the time the screen starts, the unregulated gene might be reduced again. Other screening assays are discussed elsewhere herein.

In an embodiment, the invention provides a cell from or of an in vitro method of delivery, wherein the method comprises contacting the delivery system with a cell, optionally a eukaryotic cell, whereby there is delivery into the cell of constituents of the delivery system, and optionally obtaining data or results from the contacting, and transmitting the data or results.

In an embodiment, the invention provides a cell from or of an in vitro method of delivery, wherein the method comprises contacting the delivery system with a cell, optionally a eukaryotic cell, whereby there is delivery into the cell of constituents of the delivery system, and optionally obtaining data or results from the contacting, and transmitting the data or results; and wherein the cell product is altered compared to the cell not contacted with the delivery system, for example altered from that which would have been wild type of the cell but for the contacting. In an embodiment, the cell product is non-human or animal. In some embodiments, the cell product is human.

In some embodiments, a host cell is transiently or non-transiently transfected with one or more vectors described herein. In some embodiments, a cell is transfected as it naturally occurs in a subject optionally to be reintroduced therein. In some embodiments, a cell that is transfected is taken from a subject. In some embodiments, the cell obtained from or is derived from cells taken from a subject, such as a cell line. Delivery mechanisms and techniques of the targeting moieties, polypeptides, polynucleotides, vectors, vector systems, particles, viral (e.g., AAV) particles, cells, or any combination thereof described herein.

In some embodiments, it is envisaged to introduce one or more of the engineered targeting moieties, polypeptides, polynucleotides, vectors, vector systems, particles, viral (e.g., AAV) particles, cells, or any combination thereof described herein directly to the host cell. For instance, the engineered AAV capsid system molecule(s) can be delivered together with one or more cargo molecules to be packaged into an engineered AAV particle.

In some embodiments, the invention provides a method of expressing an engineered delivery molecule and cargo molecule to be packaged in an engineered viral (e.g., AAV) particle in a cell that can include the step of introducing the vector according any of the vector delivery systems disclosed herein.

### Receptor Screening

Described in certain example embodiments herein are assays and methods for screening and identifying cell and tissue surface receptors that facilitate transduction by one or more of the CNS specific targeting moieties of the present invention. In some embodiments, such a method can be based upon an RNAi, CRISPR activation (CRISPRa), CRISPR inhibition (CRISPRi) or CRISPR knockdown or knockout approach. In some embodiments, such a method can be based upon a small molecule library screening.

In some embodiments, the method includes contacting one or more cells with a CRISPRa, CRISPRi, or CRISPRkd/ko system or component thereof thereby increasing or decreasing expression of genes to which the system is targeted and transducing the one or more cells with a composition comprising a targeting moiety effective to target a CNS cell of the present invention, and detecting, quantifying, or otherwise measuring transduction efficiency of the composition a targeting moiety effective to target a CNS cell of the present invention to determine or otherwise identify genes, pathways, programs, receptors, and/or the like involved with or that mediates transduction of the compositions comprising a targeting moiety effective to target a CNS cell of the present invention and/or are capable of enhancing and/or reducing transduction by one or more of the compositions comprising a targeting moiety effective to target a CNS cell of the present invention. In some embodiments, the CRISPRa, CRISPRi, CRISPRkd/ko system comprises a dCas, such as a dCas9, dCas12, or other inactive Cas which are described in greater detail elsewhere herein. In some embodiments the CRSIPRi system comprises a dCas12 General principles of CRISPRa, CRISPRi, and CRISPRko/kd screens are known in the art. See also e.g., Chong et al., Trends Cell Biol. 2020 August; 30(8):619-627; Ramkumar et al., Blood Adv. 2020 Jul. 14; 4(13):2899-2911; Semesta et al., PLoS Genet. 2020 Oct. 14; 16(10); Kampamann et al., ACS Chem Biol. 2018 Feb. 16; 13(2):406-416; Sanson et al., Nat Commun. 2018 Dec. 21; 9(1):5416; Gilbert et al., Cell. 2014 Oct. 23; 159(3):647-61; Tian et al., Neuron. 2019 Oct. 23; 104(2):239-255.e12; Tian et al., Nat Neurosci. 2021 July; 24(7):1020-1034; Kampmann et al., Nat Rev Neurol. 2020 September; 16(9):465-480; Schuster et al., Trends Biotechnol. 2019 January; 37(1):38-55; Dominguez et al., Nat Rev Mol Cell Biol. 2016 January; 17(1):5-15; Dudek et al., Mol Ther. 2020 Feb. 5; 28(2):367-381; Chow and Chen. Trends Cancer. 2018 May; 4(5):349-358, Hanna and Doench. Nat Biotechnol. 2020 July; 38(7):813-823, Qi et al., Cell. 152(5):1173-1183 (2013); the teachings of which can be adapted for use with the present invention.

In some embodiments, the method includes contacting one or more cells with one or more small molecules, such as a small molecule or chemical library in which the small molecules contained in the library have known effects on particular cell surface molecules and/or receptors, optionally those known to be involved with viral, and more particularly AAV, transduction, and transducing composition a targeting moiety effective to target a CNS cell of the present invention and detecting, quantifying, or otherwise measuring transduction efficiency of the composition a targeting moiety effective to target a CNS cell of the present invention to determine or otherwise identify cell surface molecules and/or receptors and/or the like involved with or that mediates transduction of the compositions comprising a targeting moiety effective to target a CNS cell of the present invention and/or are capable of enhancing and/or reducing transduction by one or more of the compositions comprising a targeting moiety effective to target a CNS cell of the present invention.

The screening can be carried out using any suitable low or high throughput approaches, examples of which are provided elsewhere herein and are generally known in the art. In some embodiments, the screening can be done in vitro or ex vivo using cells, cell populations, organoids, tissue explants, and/or the like. In some embodiments, the screening can be done in vivo, such as via animal models, including, but not limited to mouse and non-human primates.

In some embodiments, the compositions comprising a targeting moiety effective to target a CNS cell of the present invention contain a cargo molecule that is a reporter molecule to facilitate transduction detection, quantification and measurement. Exemplary reporter cargo molecules are described in greater detail elsewhere herein.

In some embodiments, the method further includes directed evolution of viral, such as AAV, capsids based on genes, pathways, programs, cell-surface receptors and/or the like identified in a screen previously described so as to further evolve n-mer motifs to enhance transduction efficacy of the CNS targeting moieties.

The invention is further described in the following examples, which do not limit the scope of the invention described in the claims. Further embodiments are illustrated in the following Examples which are given for illustrative purposes only and are not intended to limit the scope of the invention.

### EXAMPLES

### Example 1—mRNA Based Detection Methods are More Stringent for Selection of AAV Variants

FIG. 1 demonstrates the adeno-associated virus (AAV) transduction mechanism, which results in production of mRNA. As is demonstrated in FIG. 1, functional transduction of a cell by an AAV particle can result in the production of an mRNA strand. Non-functional transduction would not produce such a product despite the viral genome being detectable using a DNA-based assay. Thus, mRNA-based detection assays to detect transduction by e.g., an AAV can be more stringent and provide feedback as to the functionality of a virus particle that is able to functionally transduce a cell. FIG. 2 shows a graph that can demonstrate that mRNA-based selection of AAV variants can be more stringent than DNA-based selection. The virus library was expressed under the control of a CMV promoter.

### Example 2—mRNA Based Detection Methods can be Used to Detect AAV Capsid Variants from a Capsid Variant Library

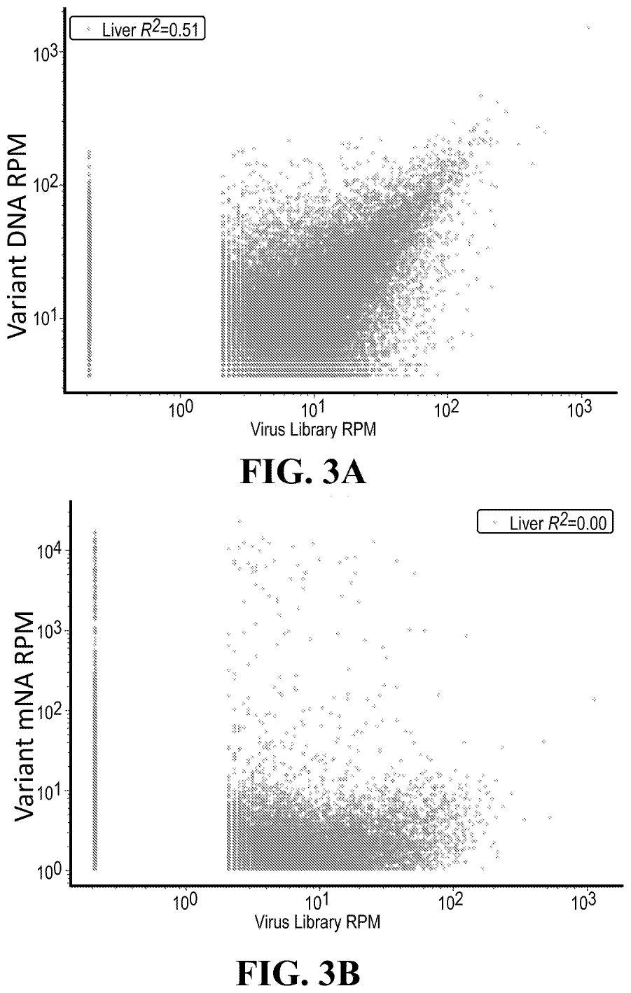

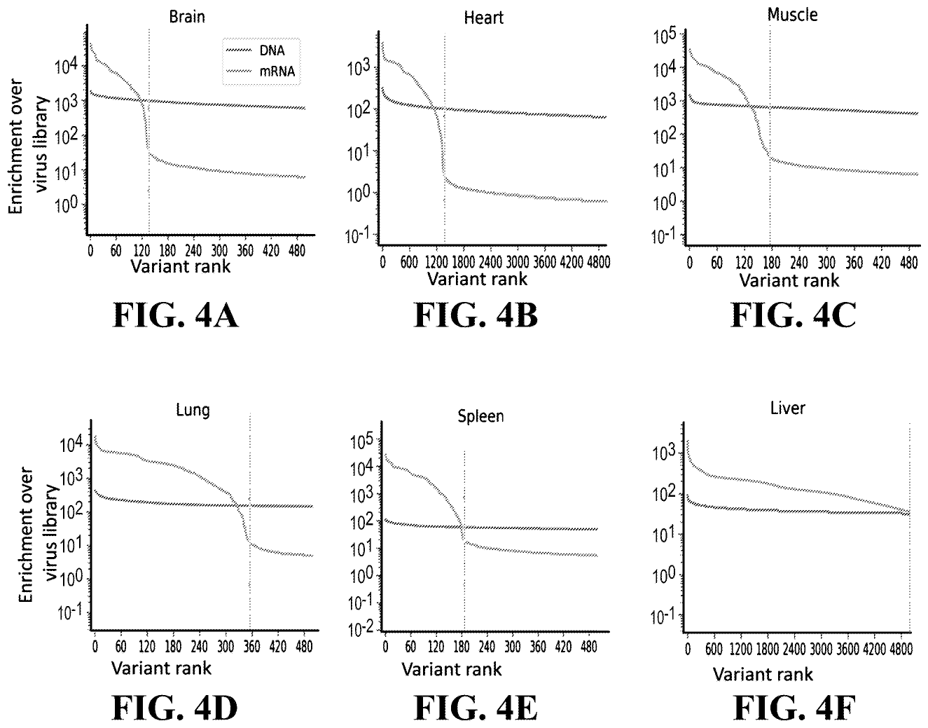

FIGS. 3A-3B show graphs that can demonstrate a correlation between the virus library and vector genome DNA (FIG. 3A) and mRNA (FIG. 3B) in the liver. FIGS. 4A-4F show graphs that can demonstrate capsid variants expressed at the mRNA level identified in different tissues.

### Example 3—Capsid mRNA Expression can be Driven by Tissue Specific Promoters

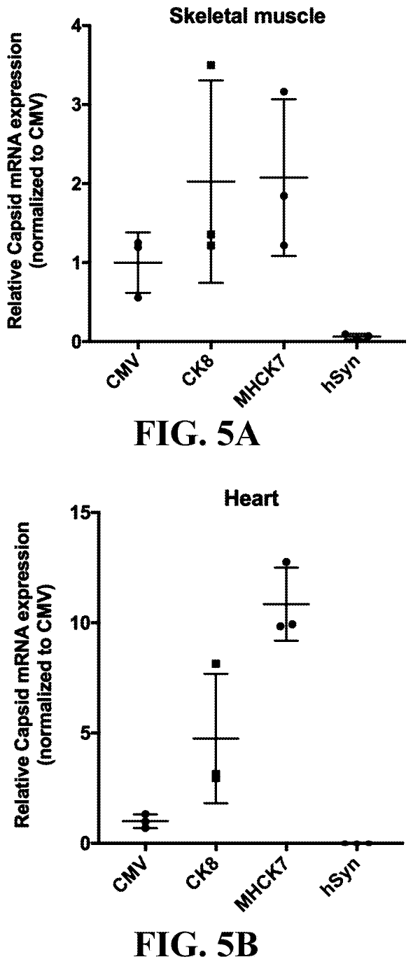

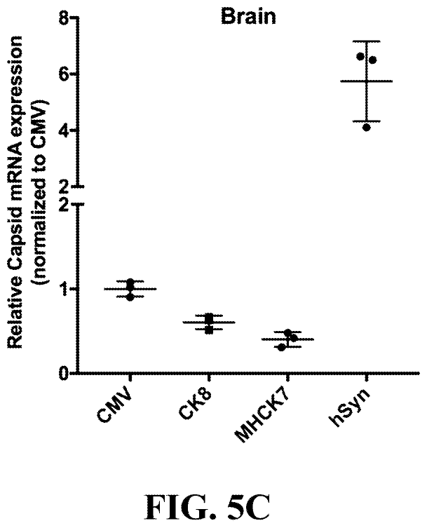

FIGS. 5A-5C show graphs that can demonstrate capsid mRNA expression in different tissues under the control of cell-type specific promoters (as noted on x-axis). CMV was included as an exemplary constitutive promoter. CK8 is a muscle-specific promoter. MHCK7 is a muscle-specific promoter. hSyn is a neuron specific promoter.

### Example 4—Capsid Variant Library Generation, Variant Screening, and Variant Identification

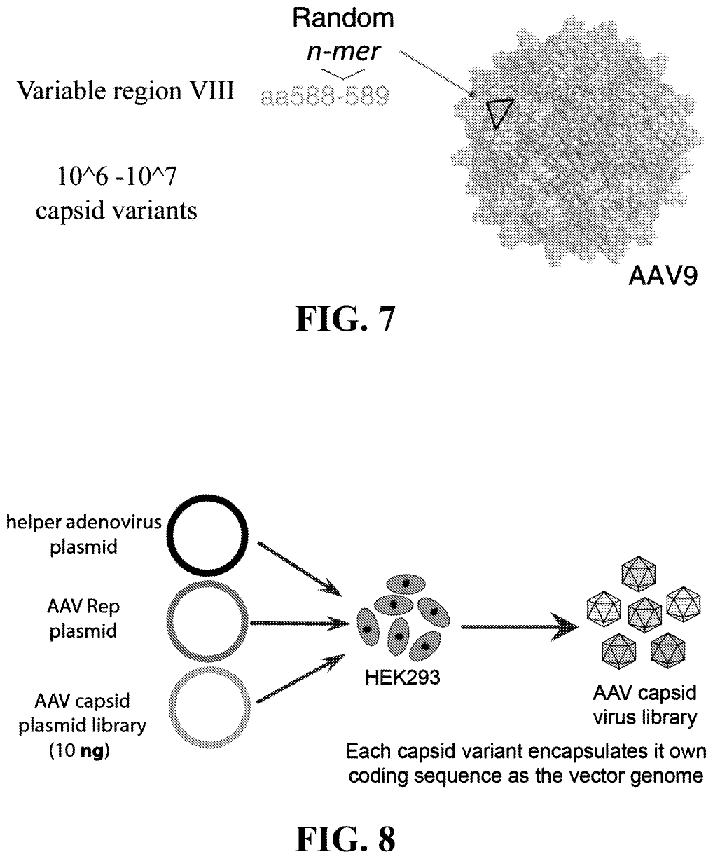

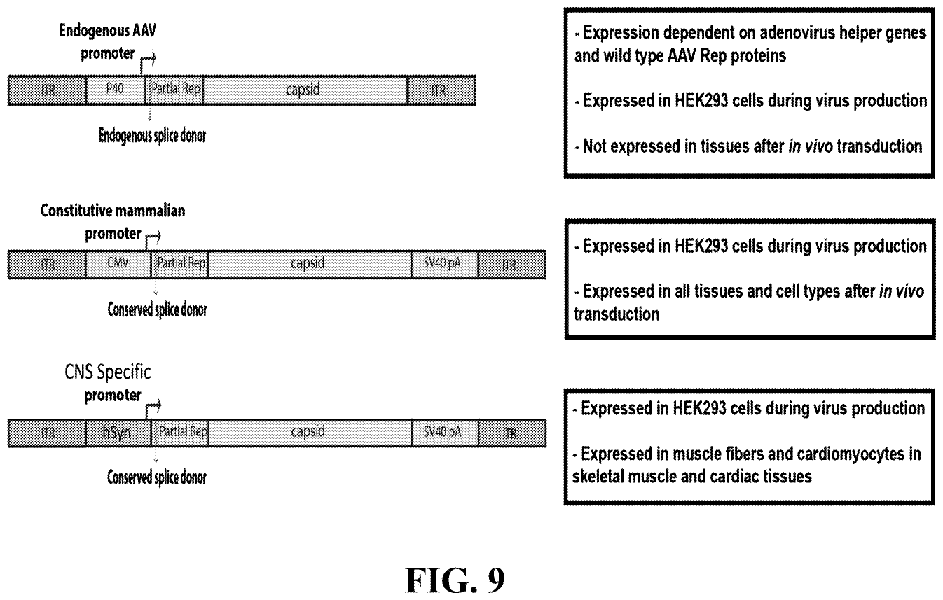

Generally, an AAV capsid library can be generated by expressing engineered capsid vectors each containing an engineered AAV capsid polynucleotide previously described in an appropriate AAV producer cell line. See e.g., FIG. 8. This can generate an AAV capsid library that can contain one more desired cell specific engineered AAV capsid variant. FIG. 7 shows a schematic demonstrating embodiments of generating an AAV capsid variant library, particularly insertion of a random n-mer (n=e.g., 3-25 or 3-15 amino acids) into a wild-type AAV, e.g., AAV9. In this example, random 7-mers were inserted between aa588-589 of variable region VIII of AAV9 viral protein and used to form the viral genome containing vectors with one variant per vector. As shown in FIG. 8, the capsid variant vector library was used to generate AAV particles where each capsid variant encapsulated its coding sequence as the vector genome. FIG. 9 shows vector maps of representative AAV capsid plasmid library vectors (see e.g., FIG. 8) that can be used in an AAV vector system to generate an AAV capsid variant library. The library can be generated with the capsid variant polynucleotide under the control of a tissue specific promoter or constitutive promoter. The library was also made with capsid variant polynucleotide that included a polyadenylation signal.

As shown in FIG. 6A the AAV capsid library can be administered to various non-human animals for a first round of mRNA-based selection. As shown in FIG. 1, the transduction process by AAVs and related vectors can result in the production of an mRNA molecule that is reflective of the genome of the virus that transduced the cell. As is at least demonstrated in the Examples herein, mRNA based selection can be more specific and effective to determine a virus particle capable of functionally transducing a cell because it is based on the functional product produced as opposed to just detecting the presence of a virus particle in the cell by measuring the presence of viral DNA.

As is further shown in FIG. 6A, after first-round administration, one or more engineered AAV virus particles having a desired capsid variant can then be used to form a filtered AAV capsid library. Desirable AAV virus particles can be identified by measuring the mRNA expression of the capsid variants and determining which variants are highly expressed in the desired cell type(s) as compared to non-desired cells type(s). Those that are highly expressed in the desired cell, tissue, and/or organ type are the desired AAV capsid variant particles. In some embodiments, the AAV capsid variant encoding polynucleotide is under control of a tissue-specific promoter that has selective activity in the desired cell, tissue, or organ.

The engineered AAV capsid variant particles identified from the first round can then be administered to various non-human animals. In some embodiments, the animals used in the second round of selection and identification are not the same as those animals used for first round selection and identification. Similar to round 1, after administration the top expressing variants in the desired cell, tissue, and/or organ type(s) can be identified by measuring viral mRNA expression in the cells. The top variants identified after round two can then be optionally barcoded and optionally pooled. In some embodiments, top variants from the second round can then be administered to a non-human primate to identify the top cell-specific variant(s), particularly if the end use for the top variant is in humans. Administration at each round can be systemic. As further shown in FIG. 6B after the second round of selection, a third round of selection, which can optionally include benchmarking against known, control, and/or standard (e.g., benchmark) variants can be performed.

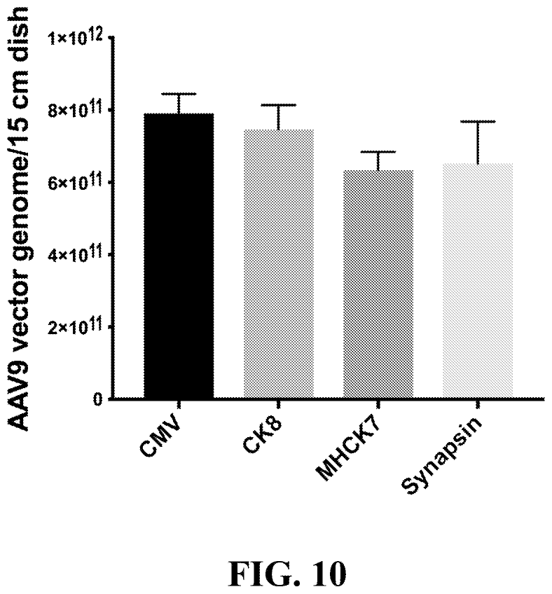

FIG. 10 shows a graph that can demonstrate the viral titer (calculated as AAV9 vector genome/15 cm dish) produced by libraries generated using different promoters. As demonstrated in FIG. 10, virus titer was not affected significantly be the use of different promoters.

### Example 5—CNS n-Mer Inserts

CNS n-mer inserts were generated as described elsewhere herein and then screened for transduction efficiency in various strains of mice (C57BL/6J and BALB/cJ). Table 1 shows the top motifs based on CNS transduction. As previously discussed, each n-mer insert's transduction efficacy in CNS cells was tested with both AQ and DG as the aa587 and aa588 (the two amino acids in the AAV immediately preceding the n-mer insert. Some exemplary n-mer inserts that stood out when preceded by AQ are KTVGTVY (SEQ ID NO: 3), RSVGSVY (SEQ ID NO: 4), RYLGDAS (SEQ ID NO: 5), WVLPSGG (SEQ ID NO: 6), VTVGSIY (SEQ ID NO: 7), VRGSSIL (SEQ ID NO: 8), RHHGDAA (SEQ ID NO: 9), VIQAMKL (SEQ ID NO: 10), LTYGMAQ (SEQ ID NO: 11), LRIGLSQ (SEQ ID NO: 12), GDYSMIV (SEQ ID NO: 13), VNYSVAL (SEQ ID NO: 14), RHIADAS (SEQ ID NO: 15), RYLGDAT (SEQ ID NO: 16), QRVGFAQ (SEQ ID NO; 17), QIAHGYST (SEQ ID NO: 18), WTLESGH (SEQ ID NO: 19), and GENSARW (SEQ ID NO: 20).

Some exemplary n-mer inserts that stood out when preceded by DG are ASNPGRW (SEQ ID NO: 22), WTLESGH (SEQ ID NO: 23), REQKKLW (SEQ ID NO: 24), ERLLVQL (SEQ ID NO: 25), RMQRTLY (SEQ ID NO: 26), and REQQKLW (SEQ ID NO: 21). Engineered AAVs including a CNS n-mer of Table 1 demonstrated the ability to specifically transduce CNS cells in both strains of mice, which is in contrast to the commonly used in the art CNS AAV. Without being bound by theory, this observation can demonstrate that the engineered AAVs containing an CNS-specific n-mer insert described herein can operate through a different receptor on the surface of CNS cells than the conventional AAV used in the art to achieve CNS specificity. Given that n-mer inserts preceded by AQ with top scores did not necessarily perform the same when preceded by DG can suggest that the 3D structure of the capsid conferred by the n-mer and its interaction with endogenous AAV amino acids can influence the ability of the engineered AAV capsid to transduce a cell and thus, without being bound by theory, can play a role in contributing to the cell-type specificity of the engineered capsids.

### Example 6—Exemplary CNS n-Mer Inserts in Non-Human Primates

CNS n-mer inserts were generated as described elsewhere herein and then screened for transduction efficiency in non-human primates. Tables 2-3 show the top n-mer inserts. A general motif was observed across the very top hits (Table 3). The motif observed was P-motif having the formula amino acid sequence PX₁QGTX₂R, (SEQ ID NO: 317) wherein X₁ and X₂ are each selected from any amino acid. Exemplary n-mer insert variants containing a P-motif are shown in Table 3.

### Example 7—Benchmarking

As shown in FIGS. 6A-6B shows a general schematic for selecting CNS specific capsid, which includes a benchmarking round which evaluates the performance of selected capsids against currently used capsids for, e.g., delivery to the CNS. Table 67 shows the selected capsids used in the benchmarking round of selection. Four variants developed using selection in mice and 8 using selection in NHPs were used for benchmarking. For benchmarking here, capsid variant specific barcodes were included with each variant. Viral particles for each capsid variant were produced individually and viral particles were then pooled. Such barcoding and pooling methodology is described in greater detail elsewhere herein and applied in this context. Pooled viral particles were then injected systemically (via I.V. administration) to the periphery of different mouse strains (C57BL/6J (“(C57”) and BALB/c (“BALBc”)) and non-human primates (Macaques) so that the ability for the capsid variants to cross the blood brain barrier in different species could be evaluated. Included in the benchmarking were both engineered capsid variants from mouse and non-human primate selection (rounds 1 and/or 2) and currently used capsid variants (AAV-CAP-B10, AAV-CAP-B22, and AAV-PhP.22). mRNA and DNA corresponding to the capsid variants in various tissues were then examined to determine the CNS, strain, and species specificity of the capsid variants.

TABLE 6

| Capsid variant | Insert sequence | SEQ ID NO: |
|---|---|---|
| Mouse variant 1 | RSVGSVY | 318 |
| Mouse variant 2 | KTVGTVY | 319 |
| Mouse variant 3 | WVLPSGG | 320 |
| Mouse variant 4 | (DG)REQQKLW | 321 |
| NHP variant 1 | PTQGTVR | 322 |
| NHP variant 2 | PSQGTLR | 323 |
| NHP variant 3 | PTQGTLR | 324 |
| NHP variant 4 | RVDPSGL | 325 |
| NHP variant 5 | VVSDYTV | 326 |
| NHP variant 6 | TDALTTK | 327 |
| NHP variant 7 | STIPTMK | 328 |
| NHP variant 8 | PTQGTFR | 329 |

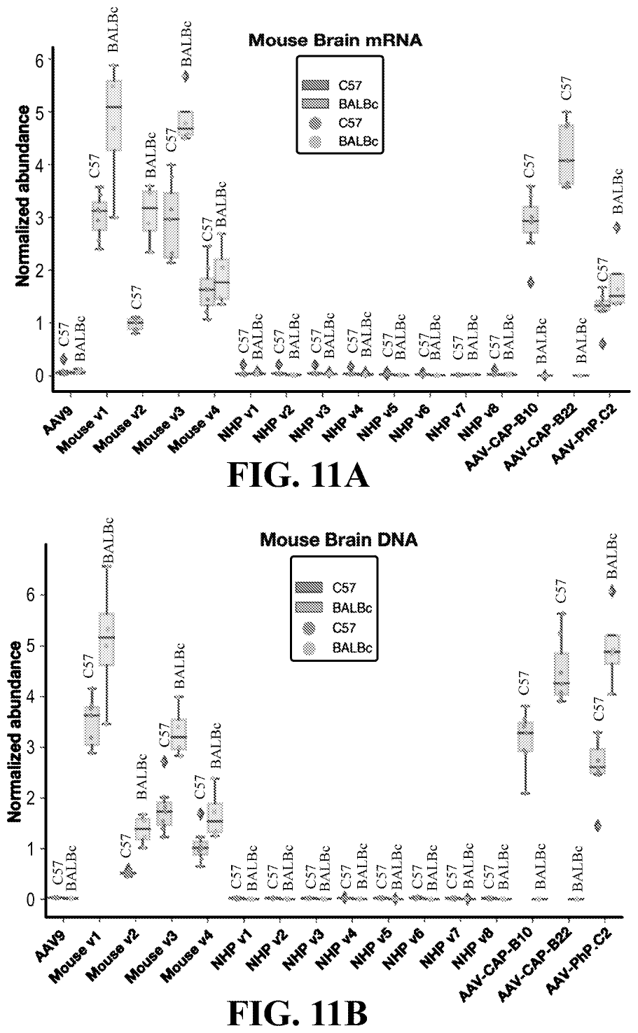

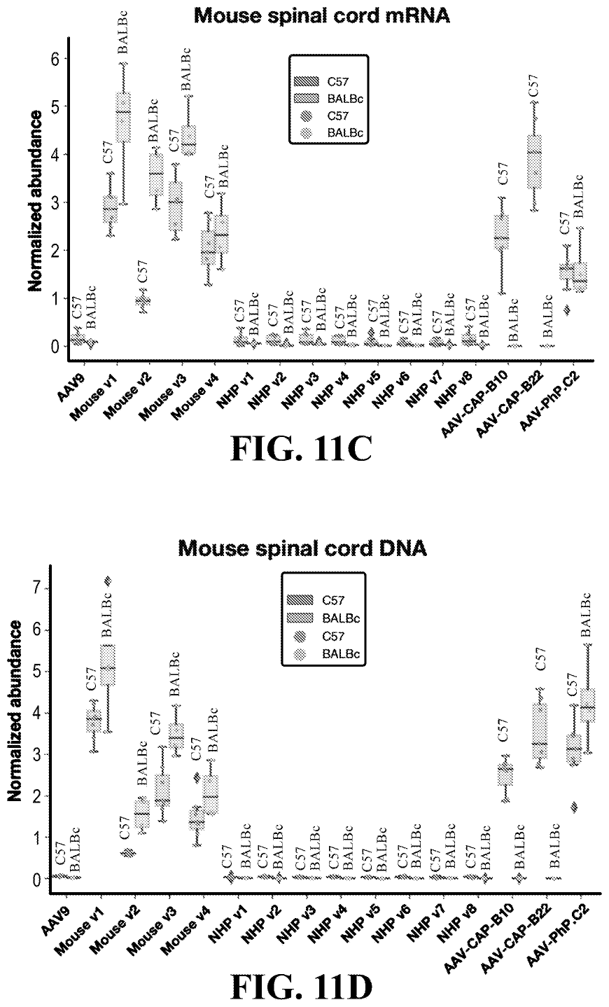

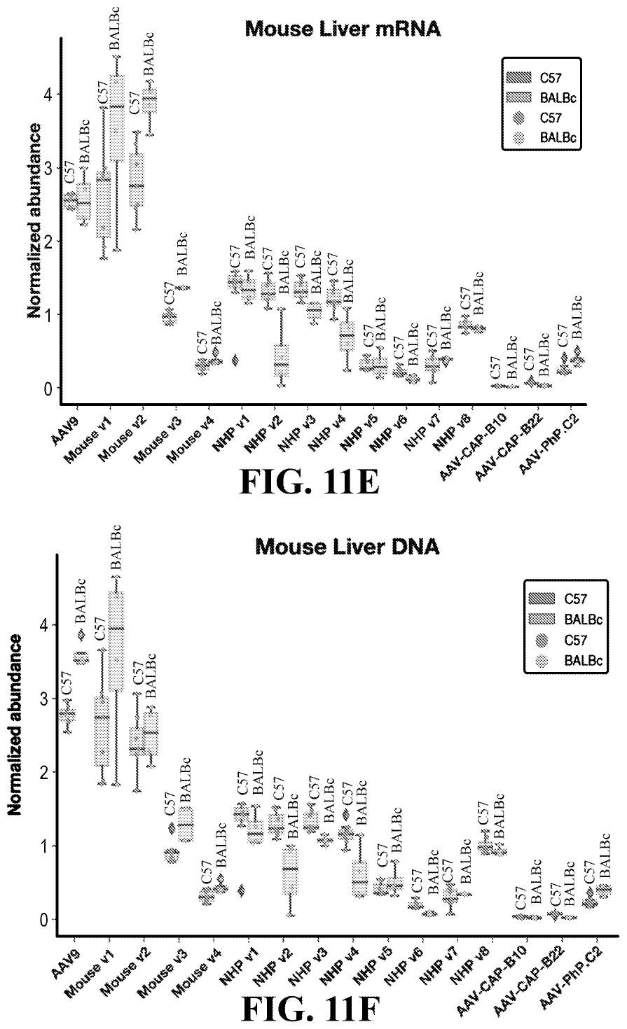

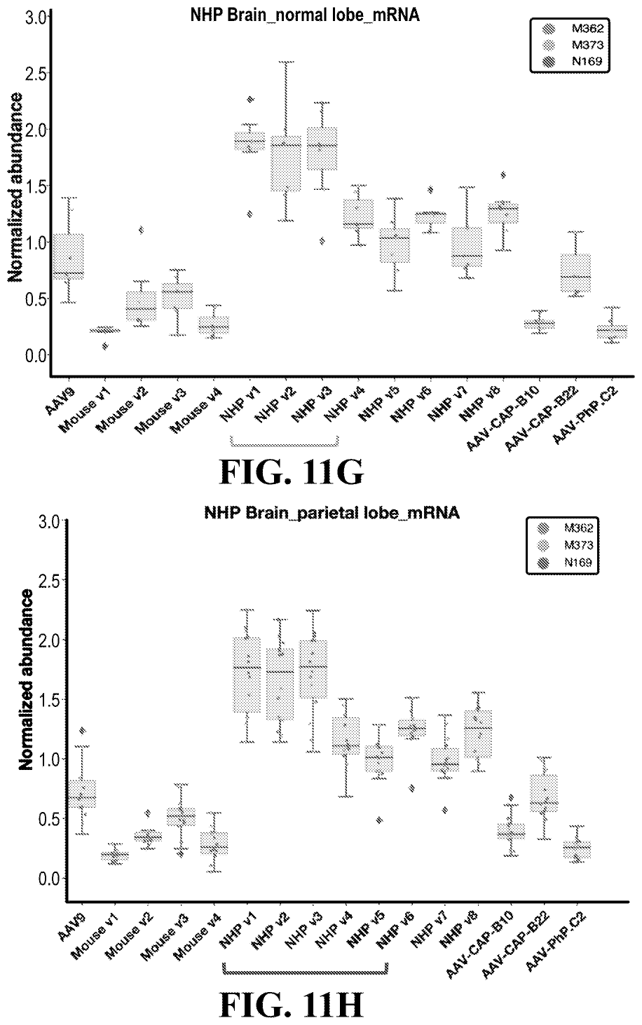

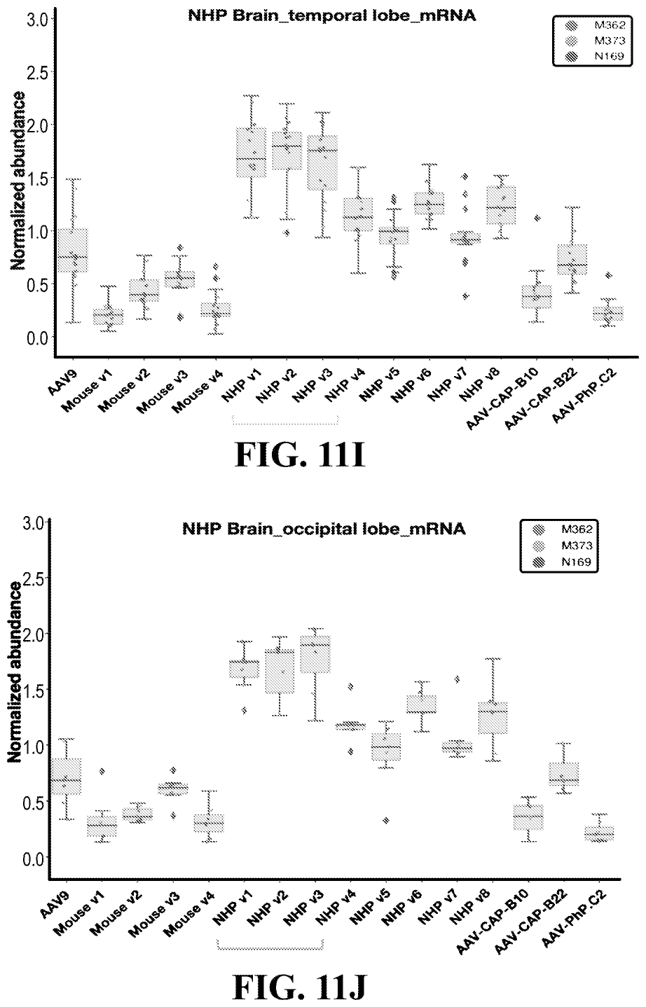

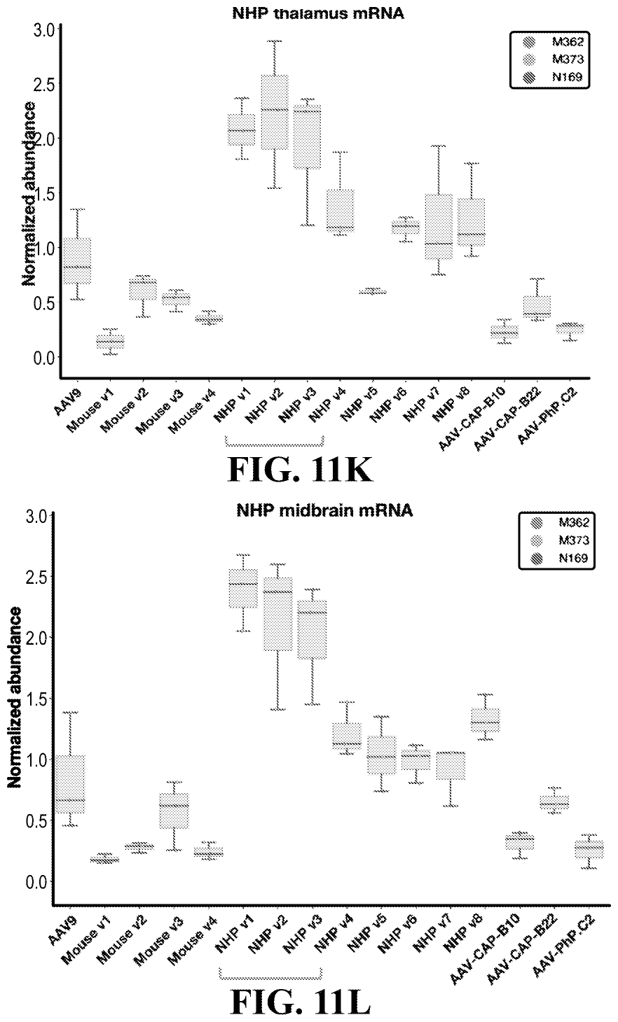

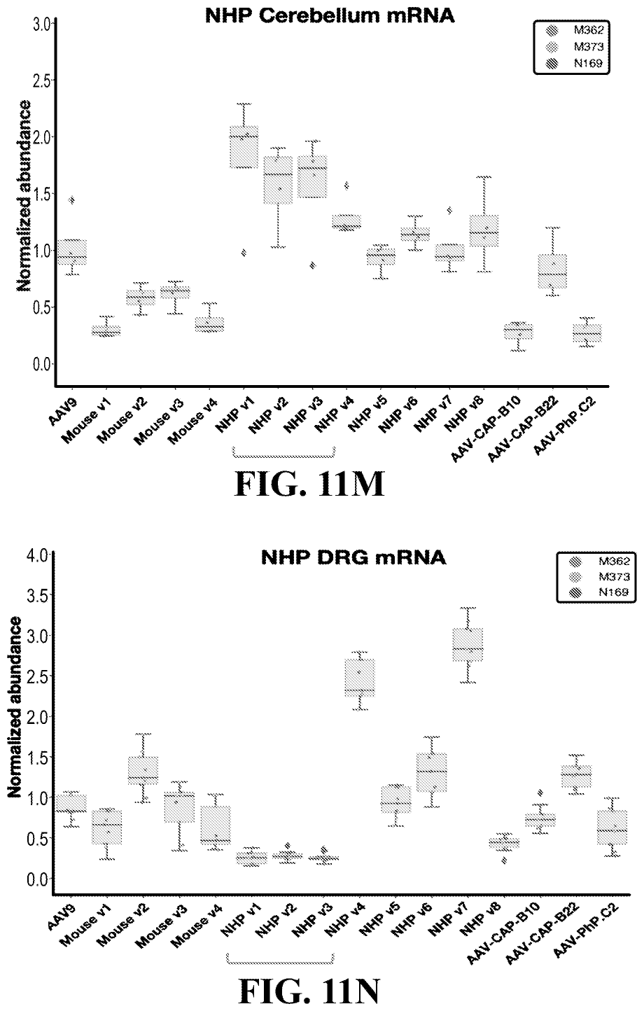

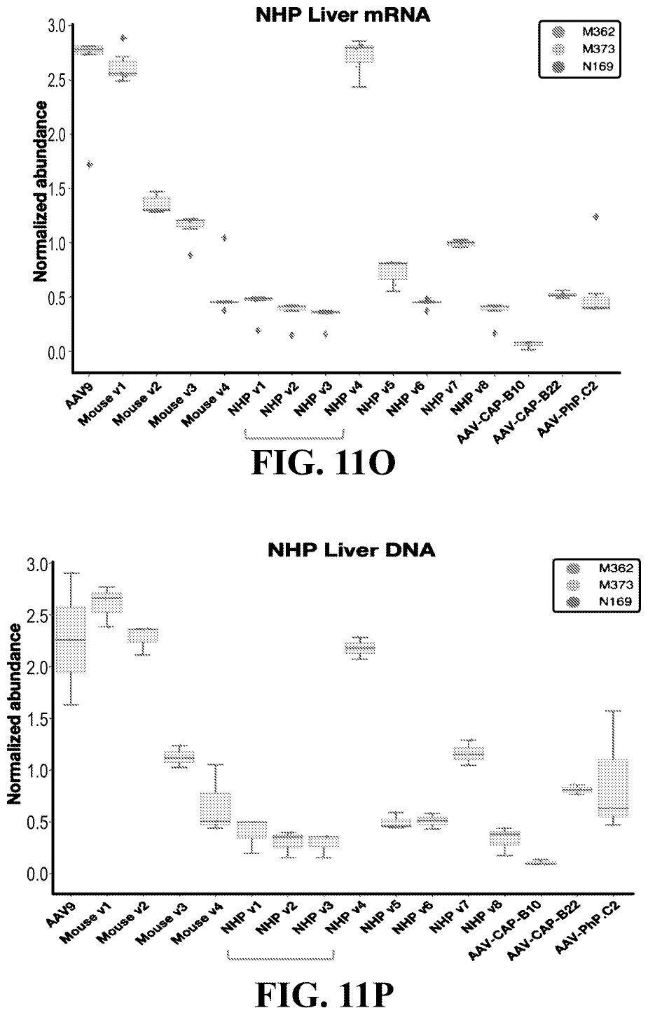

FIGS. 11A-11P show results from benchmarking the top selected capsids out of the second round of selection. In agreement with the literature, the AAV-CAP-B10, AAV-CAP22, and AAV-PhP.22 capsids demonstrated a species and strain preference, and importantly did not appear to perform well in non-human primates. Indeed, the NHP capsid variants developed using the methods described and benchmarked herein were successfully delivered to and expressed in one or more CNS tissues. Further, several NHP capsid variants tested here showed increased delivery to the CNS as compared to the capsid variants currently known and alleged to target the CNS and cross the blood brain barrier (AAV-CAP-B10, AAV-CAP22, and AAV-PhP.22). Further, most of the NHP variants were not observed to have strong liver delivery or expression (see e.g., FIGS. 11O and 11P). Expression in the dorsal root ganglion can lead to significant toxicity. Several NHP variants showed reduced or negligible delivery and/or expression in the dorsal root ganglion (DRG) (see e.g., FIG. 11N).

### Example 8—Optimized CNS Motif Variants

Directed capsid evolution and benchmarking is previously described in e.g., Examples 1-6. This Example demonstrates optimized capsid inserts specific for CNS in NHPs. Briefly, for these selections a library was screened with a fixed RGD motif (XXXRGDXXXX, where X is any amino acid), as well as a library containing a fixed P-family motif (XXXPXQGTXR (SEQ ID No: 1), where X is any amino acid) in non-human primates and identified the variants that were specific for only the CNS in NHPs. Table 7 provides the resulting top n-mer inserts and/or P motifs specific for CNS.

TABLE 7
Optimized CNS Capsid n-mer inserts and/or
P-motifs

| Capsid Insert Variant | SEQ ID NO: |
|---|---|
| EVGPTQGTVR | 332 |
| DYEPSQGTMR | 333 |
| SVSPGQGTYR | 334 |
| AVTPIQGTIR | 335 |
| ENVPMQGTVR | 336 |
| AASPPQGTMR | 337 |
| DQRPGQGTIR | 338 |
| NVSPQQGTMR | 339 |
| IPIPNQGTIR | 340 |
| STVPAQGTMR | 341 |
| STVPSQGTVR | 342 |
| SPIPSQGTLR | 343 |
| TTMPSQGTIR | 344 |
| SVMPAQGTLR | 345 |
| SIVPVQGTVR | 346 |
| SVTPSQGTLR | 347 |
| AVGPSQGTIR | 348 |
| SINPSQGTIR | 349 |
| AINPTQGTLR | 350 |
| ALLPNQGTVR | 351 |
| ASMPQQGTIR | 352 |
| SNAPAQGTMR | 353 |
| LNVPVQGTVR | 354 |
| QVTPTQGTVR | 355 |
| LVSPAQGTMR | 356 |
| AVTPSQGTIR | 357 |
| VAGPSQGTLR | 358 |
| EKLPSQGTLR | 359 |
| SISPLQGTVR | 360 |
| ESRPLQGTYR | 361 |
| NANPGQGTVR | 362 |
| IPLPSQGTVR | 363 |
| MPMPNQGTVR | 364 |
| ETRPDQGTVR | 365 |
| TEKPMQGTER | 366 |
| GDDPLQGTSR | 367 |
| SISPGQGTLR | 368 |
| EMNPLQGTVR | 369 |
| SAEPGQGTTR | 370 |
| MNVPSQGTDR | 371 |
| ITSPTQGTNR | 372 |
| MLEPTQGTPR | 373 |
| LEPPTQGTGR | 374 |
| QNEPRQGTDR | 375 |
| SMTPVQGTVR | 376 |
| SRAPDQGTIR | 377 |
| NTQPIQGTTR | 378 |
| LSVPLQGTIR | 379 |
| SEAPGQGTVR | 380 |
| GREPGQGTYR | 381 |
| IASPVQGTPR | 382 |
| SAIPPQGTSR | 383 |
| GMLPEQGTPR | 384 |
| WDDPHQGTMR | 385 |
| AIGPGQGTMR | 386 |
| ASVPQQGTVR | 387 |
| SVQPGQGTYR | 388 |
| NAGPSQGTLR | 389 |
| MKVPEQGTMR | 390 |
| TTIPEQGTYR | 391 |
| SAIPGQGTTR | 392 |
| DNGPRQGTLR | 393 |
| TLPPVQGTMR | 394 |
| NTSPMQGTQR | 395 |
| ETSPSQGTYR | 396 |
| SATPAQGTVR | 397 |
| MATPMQGTFR | 398 |
| MNVPTQGTVR | 399 |
| SVLPEQGTMR | 400 |
| STTPIQGTMR | 401 |
| NSDPQQGTVR | 402 |
| SNTPLQGTTR | 403 |
| SDAPQQGTLR | 404 |
| SNAPIQGTMR | 405 |
| NANPGQGTMR | 406 |
| ESMPVQGTHR | 407 |
| VERPLQGTMR | 408 |
| SVSPTQGTMR | 409 |
| VVAPLQGTDR | 410 |
| SVTPLQGTIR | 411 |
| VPNPVQGTPR | 412 |
| GIWPGQGTGR | 413 |
| LPTPIQGTLR | 414 |
| ANEPRQGTVR | 415 |
| TAFPTQGTMR | 416 |
| SSAPNQGTMR | 417 |
| MESPVQGTTR | 418 |
| CTAPGQGTDR | 419 |
| VTNPTQGTYR | 420 |
| SNAPIQGTFR | 421 |
| QSTPGQGTLR | 422 |
| LVKPPQGTDR | 423 |
| AAGPMQGTNR | 424 |
| SSSPNQGTFR | 425 |
| QESPLQGTVR | 426 |
| AASPTQGTLR | 427 |
| FTAPDQGTGR | 428 |
| DNVPNQGTIR | 429 |
| YSMPTQGTVR | 430 |
| SSIPGQGTAR | 431 |
| VTIPAQGTIR | 432 |
| AHMPSQGTDR | 433 |
| YVTPPQGTLR | 434 |
| DGNPAQGTGR | 435 |
| LQNPSQGTSR | 436 |
| LQGPVQGTLR | 437 |
| STNPAQGTLR | 438 |
| LPTPIQGTMR | 439 |
| SVAPTQGTVR | 440 |
| QPSPMQGTVR | 441 |
| MTQPSQGTIR | 442 |
| SAEPNQGTTR | 443 |
| TDTPSQGTVR | 444 |
| LQQPLQGTTR | 445 |
| NTHPAQGTVR | 446 |
| VSAPMQGTMR | 447 |
| SEKPAQGTYR | 448 |
| TSLPTQGTLR | 449 |
| SERPVQGTFR | 450 |
| VLEPSQGTSR | 451 |
| ANAPIQGTIR | 452 |
| NVSPIQGTMR | 453 |
| SVLPEQGTMR | 454 |
| NDRPLQGTMR | 455 |
| VVPPGQGTLR | 456 |
| EPSPNQGTSR | 457 |
| VLLPSQGTVR | 458 |
| GSFPQQGTLR | 459 |
| NTIPVQGTQR | 460 |
| AASPPQGTLR | 461 |
| TAVPSQGTHR | 462 |
| YESPVQGTVR | 463 |
| AALPSQGTLR | 464 |
| SRIPDQGTIR | 465 |
| MPRPDQGTMR | 466 |
| ISTPTQGTLR | 467 |
| VDIPMQGTLR | 468 |
| NMLPTQGTIR | 469 |
| TVLPGQGTIR | 470 |
| TTDPVQGTVR | 471 |
| SVTPVQGTSR | 472 |
| FPLPSQGTVR | 473 |
| EMAPNQGTSR | 474 |
| VDRPSQGTMR | 475 |
| NTEPPQGTDR | 476 |
| MAMPPQGTLR | 477 |
| ATLPSQGTLR | 478 |
| LAIPPQGTSR | 479 |
| TSGPVQGTFR | 480 |
| SSGPGQGTDR | 481 |
| MVTPGQGTMR | 482 |
| EGTPVQGTTR | 483 |
| MPIPSQGTPR | 484 |
| NPLPTQGTSR | 485 |
| SHFPPQGTNR | 486 |
| AVTPTQGTIR | 487 |
| ESGPSQGTSR | 488 |
| MTVPSQGTFR | 489 |
| EAYPTQGTIR | 490 |
| SSTPAQGTFR | 491 |
| DSRPLQGTIR | 492 |
| DNAPLQGTNR | 493 |
| QPIPPQGTMR | 494 |
| ASSPTQGTER | 495 |
| NVSPSQGTVR | 496 |
| IHLPAQGTVR | 497 |
| SSVPAQGTQR | 498 |
| TTGPNQGTLR | 499 |
| ATGPTQGTLR | 500 |
| AATPGQGTYR | 501 |
| AAVPTQGTVR | 502 |
| EGKPEQGTTR | 503 |
| SIAPTQGTIR | 504 |
| DVRPSQGTIR | 505 |
| MLRPEQGTDR | 506 |
| TVSPTQGTTR | 507 |
| KERPEQGTMR | 508 |
| DSSPNQGTYR | 509 |
| SLAPMQGTTR | 510 |
| ELHPTQGTSR | 511 |
| ETGPMQGTVR | 512 |
| VLAPVQGTQR | 513 |
| KEAPDQGTGR | 514 |
| ASEPSQGTQR | 515 |
| IYGPNQGTLR | 516 |
| TERPVQGTFR | 517 |
| GATPLQGTLR | 518 |
| LAGPMQGTIR | 519 |
| EVRPIQGTVR | 520 |
| MANPIQGTVR | 521 |
| TREPQQGTFR | 522 |
| MKDPIQGTYR | 523 |
| LSEPPQGTLR | 524 |
| LMEPRQGTVR | 525 |
| VASPMQGTSR | 526 |
| QTFPNQGTMR | 527 |
| MNRPTQGTER | 528 |
| EVPPSQGTLR | 529 |
| VTGPPQGTYR | 530 |
| SHVPAQGTMR | 531 |
| MVMPVQGTVR | 532 |
| SDKPVQGTMR | 533 |
| SVAPTQGTIR | 534 |
| GTTPDQGTMR | 535 |
| GSEPNQGTYR | 536 |
| GPMPIQGTLR | 537 |
| NQMPMQGTAR | 538 |
| GNTPVQGTVR | 539 |
| SANPLQGTIR | 540 |
| MSFPSQGTHR | 541 |
| NPDPIQGTIR | 542 |
| EMNPVQGTNR | 543 |
| TVLPNQGTVR | 544 |
| VIQPVQGTVR | 545 |
| QTFPEQGTMR | 546 |
| VLTPSQGTTR | 547 |
| NNGPMQGTVR | 548 |
| VETPNQGTHR | 549 |
| SLVPNQGTVR | 550 |
| DSAPHQGTYR | 551 |
| DHGPSQGTSR | 552 |
| AAMPGQGTVR | 553 |
| ISSPGQGTDR | 554 |
| NVSPSQGTLR | 555 |
| SSIPIQGTSR | 556 |
| GSVPGQGTTR | 557 |
| MREPSQGTSR | 558 |
| TDQPSQGTVR | 559 |
| MMQPVQGTSR | 560 |
| SFQPGQGTLR | 561 |
| MNAPSQGTTR | 562 |
| NEVPTQGTAR | 563 |
| VSGPEQGTSR | 564 |
| GYEPAQGTMR | 565 |
| SLIPDQGTIR | 566 |
| DYGPSQGTVR | 567 |
| TELPMQGTVR | 568 |
| SYMPLQGTVR | 569 |
| DYKPNQGTVR | 570 |
| DTKPNQGTVR | 571 |
| MNTPAQGTLR | 572 |
| VMNPEQGTAR | 573 |
| EILPGQGTLR | 574 |
| MPSPAQGTIR | 575 |
| NVIPEQGTNR | 576 |
| QMEPHQGTTR | 577 |
| FVVPDQGTNR | 578 |
| ENNPGQGTTR | 579 |
| NWKPEQGTDR | 580 |
| SVSPNQGTIR | 581 |
| DPSPLQGTDR | 582 |

### Example 9—Comparison of the EVGPTQGTVR (SEQ ID NO: 332) Capsid Insert Variant with AAV9 in NHP Tissues

This Example compares the transduction and vector genome distribution of the top hit (EVGPTQGTVR (SEQ ID NO: 332, Table 7) from the screen discussed in Example 8 and AAV9.

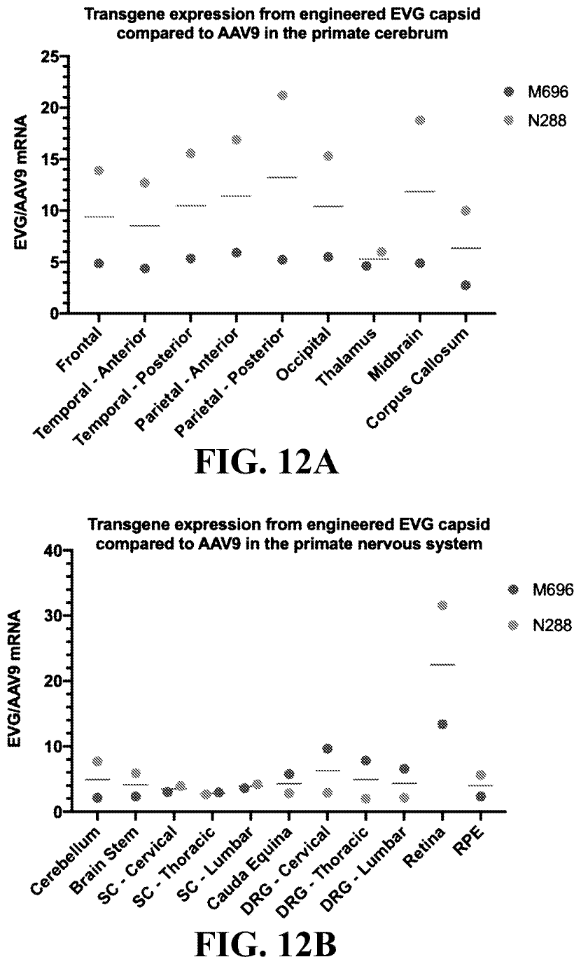

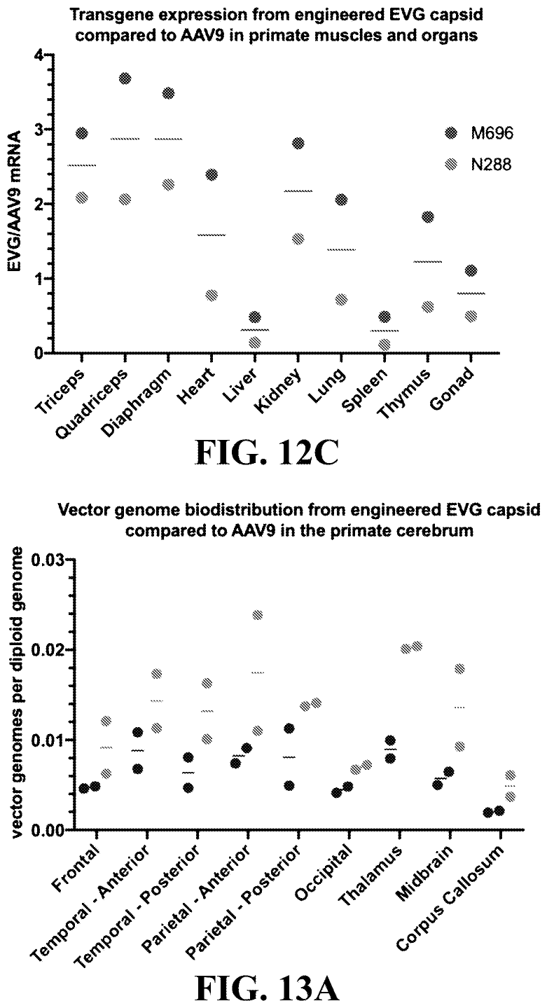

FIGS. 12A-12C show a comparison of transduction between the EVGPTQGTVR (SEQ ID NO: 332, Table 7) capsid insert variant with AAV9 in NHP tissues. FIG. 12A shows transgene expression from the engineered EVG capsid containing the EVGPTQGTVR (SEQ ID NO: 332) capsid insert variant compared to AAV9 in the primate cerebrum. FIG. 12B shows transgene expression from the engineered EVG capsid containing the EVGPTQGTVR (SEQ ID NO: 332) capsid insert variant compared to AAV9 in the primate nervous system. FIG. 12C shows transgene expression from the engineered EVG capsid containing the EVGPTQGTVR (SEQ ID NO: 332) capsid insert variant compared to AAV9 in various primate muscles and organs.

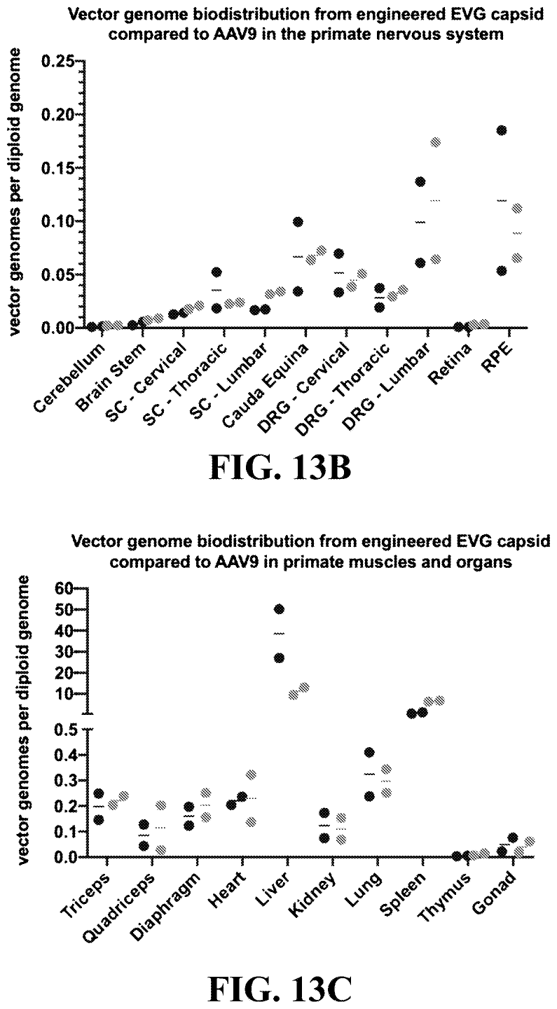

FIGS. 13A-13C show a comparison of the vector genome biodistribution between the EVGPTQGTVR (SEQ ID NO: 332) capsid insert variant with AAV9 in NHP tissues. FIG. 13A shows the vector genome biodistribution from the engineered EVG capsid containing the EVGPTQGTVR (SEQ ID NO: 332) capsid insert variant compared to AAV9 in the primate cerebrum. FIG. 13B shows the vector genome biodistribution from the engineered EVG capsid containing the EVGPTQGTVR (SEQ ID NO: 332) capsid insert variant compared to AAV9 in the primate nervous system. FIG. 13C shows the vector genome biodistribution from the engineered EVG capsid containing the EVGPTQGTVR (SEQ ID NO: 332) capsid insert variant compared to AAV9 in various primate muscles and organs.

### Example 10

### Introduction

Recombinant adeno-associated virus (rAAV) vectors are the vehicle of choice for gene therapy applications in the central nervous system (CNS) due to their low immunogenicity and ability to facilitate long-term gene expression in both dividing and non-dividing cells.¹⁻⁶ Clinical and preclinical studies of rAAV-based therapies with naturally occurring AAV serotypes have shown promise in the treatment of a variety of CNS disorders.^{1,5,7-11} However, the efficacy of rAAVs in transducing the CNS has been limited by the protective effect of the blood-brain barrier (BBB) and the broad tissue tropism of naturally occurring AAV serotypes, which together result in inefficient transduction of target cell populations in the CNS.^{1,2,12} Direct administration of rAAVs into the CNS, such as via intrathecal, intracisternal, or intraparenchymal injection, is a commonly employed strategy to bypass the BBB.^{1,2,5,13} However, these delivery routes generally do not result in widespread and uniform transduction of the CNS and can be associated with considerable surgical risk.^{2,13}

The discovery that the AAV9 serotype can cross the BBB has introduced the possibility of utilizing noninvasive systemic administration of rAAVs via the vascular system to facilitate widespread transduction across the CNS.^{1,2,13,14} Intravenous (IV) infusion has been employed in a number of clinical trials of CNS-targeted rAAV therapies^{1,11} and is the administration route of choice for an FDA-approved treatment for spinal muscular atrophy.⁷ However, systemic administration of naturally occurring AAV serotypes is complicated by sequestration of viral particles in the liver and the protective effect of the BBB, both of which limit rAAV bioavailability in the CNS.^{1,2,12,15,16} Achieving therapeutic efficacy in the CNS with systemic administration of rAAVs therefore requires large doses, sometimes exceeding 1E+14 vector genomes per kilogram body mass (vg/kg).^{1,2,5,13} In addition to posing significant manufacturing challenges, high dose rAAV therapy compounds the safety risk associated with an immune response in the liver, a phenomenon that has been observed in both clinical and preclinical studies.^{1,2,15,17-20}

Engineering AAV capsids that display both enhanced transduction of the CNS and reduced transduction in peripheral organs following systemic administration will facilitate the development of CNS-targeted therapies with improved safety and efficacy at a reduced dose. Previous studies have successfully applied directed evolution techniques to generate novel AAV capsids with CNS-tropic properties in vivo,²¹⁻²⁵ though translating these findings from mouse models to nonhuman primates (NHPs) has proved challenging and has complicated efforts to develop capsids with therapeutic potential in humans. A directed evolution strategy performed in Cre-transgenic C57BL/6J mice using the CREATE (Cre recombination-based AAV targeted evolution) method yielded potent CNS-tropic variants such as PHP.B and PHP.eB.^{21,22} However, the CNS-tropic properties of these variants translate poorly even to other mouse strains, and studies assessing intravenous administration of PHP.B in marmosets found that it failed to outperform AAV9 in CNS transduction.²⁶⁻²⁹ These findings cast doubt on the applicability of such vectors to human gene therapy and highlight the need to evaluate novel capsids in NHPs.

Recent studies have attempted to find less strain-specific CNS-tropic capsids using a multiplexed CREATE strategy in which directed evolution is performed across multiple mouse strains.²³ Two PHP.eB-related variants identified in these efforts, AAV.CAP-B10 and AAV.CAP-B22, were later found to have improved CNS transduction in the marmoset brain compared to AAV9.²⁵ Though variants demonstrating efficacy in marmosets likely hold greater therapeutic potential than those only capable of transducing the mouse brain, marmosets are much smaller and more evolutionarily distant from humans than are other common NHP models such as macaques. Given that positive results for certain engineered rAAVs in mice do not necessarily translate to NHPs^{25,26,29} and the extensibility of transduction data in marmosets to other NHPs is unknown, it is of utmost importance to assess the performance of novel rAAVs in appropriate animal models in order to identify candidate vectors for human gene therapy applications.

The unrealized potential of systemically administered rAAVs with CNS-tropic engineered capsids combined with the challenges in translating these capsids to NHPs serve as motivation for this work. In contrast to previous attempts to identify engineered capsids with therapeutic potential in the CNS, which typically involve selecting CNS-tropic capsids in mice, in this Example Applicant used an mRNA-based directed evolution strategy in both mice and cynomolgus macaques. This Example identifies capsids that (i) retain CNS-tropic behavior across multiple animal models such that their properties may be conserved throughout the lineage; and (ii) have CNS-tropic behavior in NHP models that are the closest practical evolutionary neighbors to humans. In both cases, Applicant seeks to identify capsids with the highest degree of translational and therapeutic potential in humans.

### Results

In Vivo mRNA-Based Selection for CNS-Tropic AAVs.

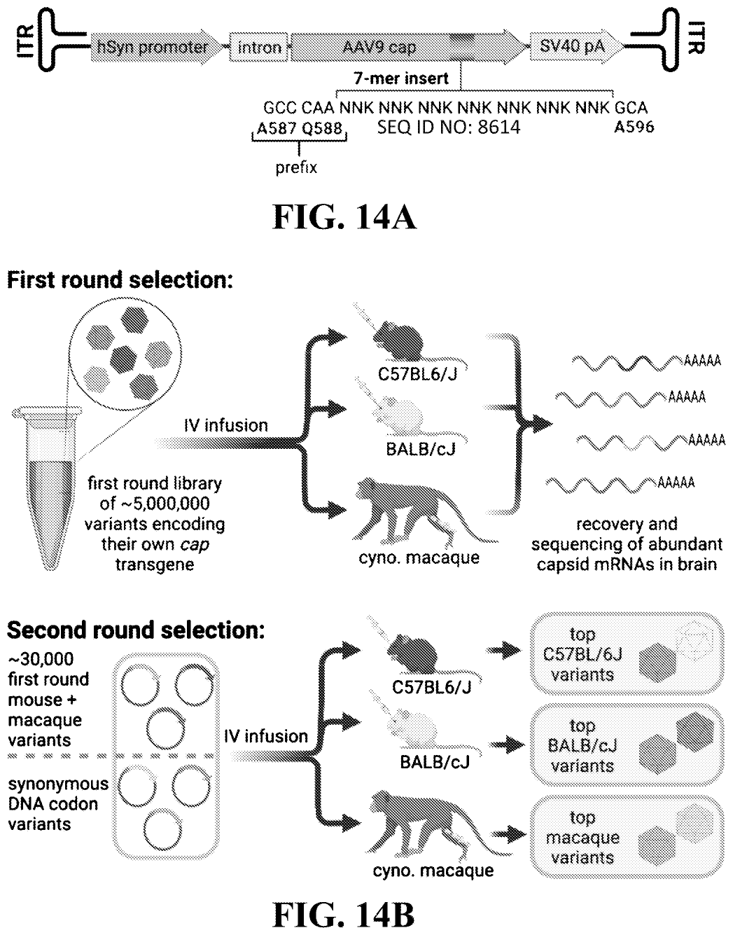

Applicant developed AAV vectors with CNS-tropic properties in mice using the previously described in vivo directed evolution strategy DELIVER (directed evolution of AAV capsids leveraging in vivo expression of transgene RNA).³⁰ As the success of capsid variants in DELIVER is based on transgene mRNA expression, it preferentially selects for variants that are able to transcribe in addition to deliver genetic cargo. Applicant first generated AAV9-based capsid libraries with a random 7-mer peptide inserted in the VR-VIII hypervariable region between residues Q588 and A589, a location known to permit exposure of the peptide on the capsid surface.^{31,32} The capsid library construct was flanked by inverted terminal repeats (ITRs), thereby eliciting self-packaging of the cap gene; that is, each capsid variant encodes its own coding sequence as a transgene. To introduce selective pressure favoring capsid variants that preferentially transduce neurons, we placed the transgene under the control of the neuron-specific human synapsin 1 promoter (hSyn) (FIG. 14A).

Applicant performed two rounds of in vivo selection in parallel in C57BL6J and BALB/cJ mice and cynomolgus macaques using expression of transgene mRNA as the selection criteria. The first round of selection included a starting library of capsids with random 7-mer inserts. To create a library for our second round of selection, Applicant identified the top 30,000 most enriched capsid variants in the brain, drawing 10,000 high-scoring variants from mice and 20,000 from macaques. Applicant next introduced a synonymous codon control where each of the 30,000 top peptides were encoded both by their experimentally recovered DNA sequence and by a synonymous DNA codon sequence (FIG. 14B). Applicant also generated a complementary library where the two residues upstream of the 7-mer peptide insert were changed from AQ to DG, given that this is a modification thought to be responsible for the enhanced CNS-tropic properties of the engineered variant PHP.eB in mice.²²

For the second round of selection in mice, we injected both the AQ and DG second-round libraries into separate sets of C57BL/6J and BALB/cJ mice. The identities of the most successful variants in mice differed depending on the prefix to the 7-mer insert (FIG. 15A-15B and Table 8). Of the variants with the wild-type AQ prefix, the four most enriched DNA sequences averaged across both mouse strains—corresponding to two pairs of synonymous peptide sequences—encoded two highly similar variants. These two variants; AQRSVGSVY (SEQ ID NO: 8587) and AQKTVGTVY (SEQ ID NO: 8588); are henceforth referred to as MDV1A and MDV1B (Mouse Double Valine), respectively (FIG. 15A), and are predicted to have similar but distinct secondary structure in the VR-VIII loop region (FIG. 15C). Of the variants with the modified PHP.eB-like DG prefix, the two most enriched DNA sequences are synonymous and encode the same peptide, DGREQQKLW (SEQ ID NO: 8589) (FIG. 17B). Applicant also recovered the sequences encoding PHP.B and PHP.eB from C57BL/6J but not BALB/cJ mice (FIG. 15B and Table 9), confirming previous findings that the CNS-tropic properties of PHP.B and PHP.eB are limited to C57BL/6J mice²⁶⁻²⁹ and further validating the DELIVER selection strategy.

Applicant chose MDV1A for further characterization in mice based on its superior performance in both the C57BL/6J and BALB/cJ strains. Applicant injected adult C57BL/6J and BALB/cJ mice of both sexes with 1E+12 vg of AAV9- or MDV1A-CMV-EGFP. Two weeks after administration of the vector, Applicant assessed vector genome delivery and transgene expression in the brain and spinal cord. MDV1A significantly outperformed AAV9 in both transgene delivery and expression in the brain of all groups of mice, demonstrating between a 25-fold and 160-fold improvement in transgene expression in the brain of male BALB/cJ and female C57BL/6J mice, respectively (FIG. 15D-15E). In the spinal cord, MDV1A significantly outperformed AAV9 in transgene delivery and expression in three of the four groups of mice, with between a 43-fold and 99-fold improvement in transgene expression in male BALB/cJ and female BALB/cJ mice, respectively (FIG. 15D-15E). In the spinal cord of female C57BL6J mice, there was a 24-fold performance difference between the two vectors that did not reach the threshold of statistical significance due to high variability in the data (FIG. 15D-15E). Immunostaining of sagittal mouse brain sections revealed greater EGFP expression from MDV1A than AAV9, with relatively uniform distribution of EGFP throughout the brain (FIG. 15F).

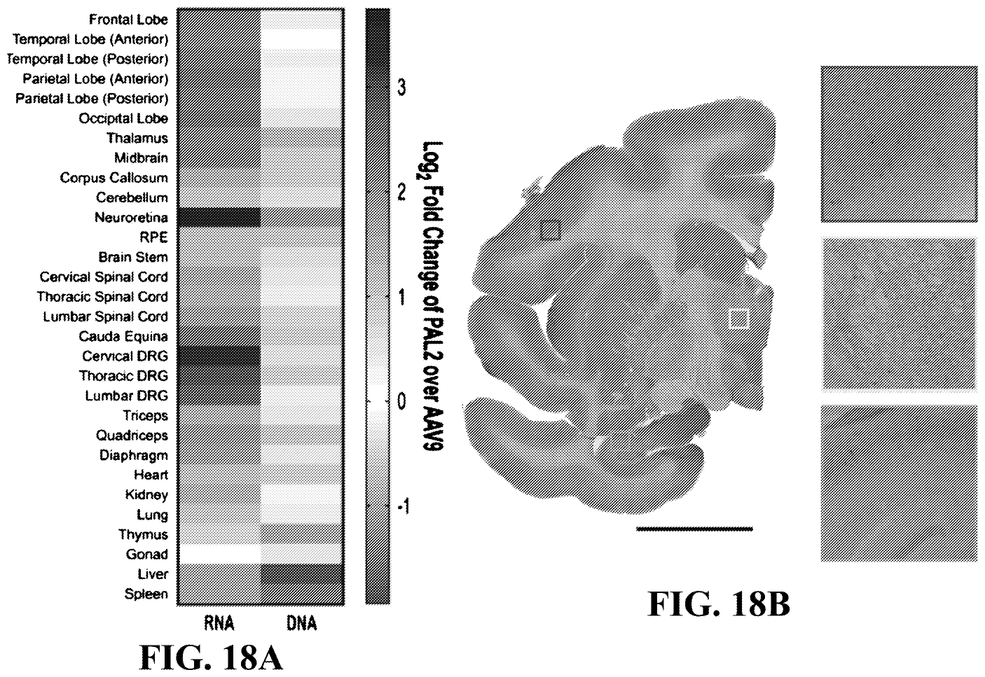

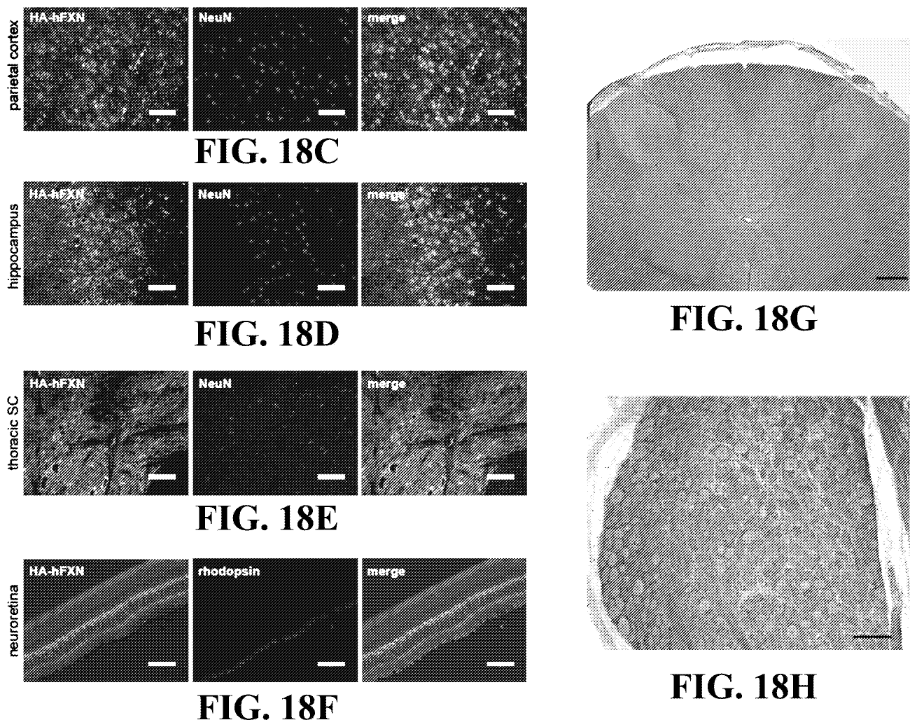

In order to select for variants with CNS-tropic activity in primates, Applicant also performed a second round of selection in three cynomolgus macaques. Applicant used the same AQ library as in the second round of selection in mice, which included variants identified in the first round in both mice and macaques. Applicant found that the variants most enriched in the macaque brain differed greatly from those identified in mice (FIG. 18A-18B). Both with and without correcting for the synonymous DNA codons, the ten most enriched variants across the entire macaque CNS were dominated by a motif typified by a proline in position 1, the string QGT in positions 3-5, and an arginine in position 7. Applicant also identified variants enriched in specific regions such as the cerebellum and spinal cord, though unlike in the CNS-wide results, these tissue-specific analyses did not converge on a single dominant motif (FIG. 19A-19B).

Applicant sought to more systematically identify sets of common motifs by performing k-medoids clustering on the top 1000 macaque variants using a dissimilarity metric based on pairwise substitution scores between 7-mer peptides. The cluster represented by the medoid sequence PTQGTLR (SEQ ID NO: 206) contained 19 variants, including 9 ranked in the top 100 sequences and 6 ranked in the top 10 (FIG. 16C). Many variants in this cluster—including most of the highest-performing variants—are broadly described by the motif PX₁QGTX₂R (SEQ ID NO: 317), where X₁ is a polar uncharged residue and X₂ is a nonpolar residue. Applicant defines the canonical Proline Arginine Loop (PAL) family of variants based on this motif, though more divergent PAL-like variants within the same cluster may share structural and functional properties with the canonical PAL variants. Computational modeling of the VR-VIII loop with the 7-mer insert predicted that canonical PAL variants share a nearly identical backbone conformation. However, even single-residue deviations from this core motif, such as the introduction of a proline at the third position in the sixth-ranked sequence PTPGTLR (SEQ ID NO: 4593), may considerably alter the backbone conformation (FIG. 16D). k-medoids identified a number of additional clusters containing high-performing variants with conserved structural properties (FIG. 19C), but many variants were sorted into singleton clusters or small clusters with only two or three variants (Table 9).

TABLE 8
Mouse AQ

| | | SEQ | | SEQ | | | |
|---|---|---|---|---|---|---|---|
| | | ID | | ID | Combined | BALB/cJ | C57BL/6J |
| Rank | Peptide | NO: | Encoding Sequence | NO: | score | score | score |
| 1 | RSVGSVY | 583 | CGGAGTGTTGGGAGTGTGTAT | 584 | 46000 | 24000 | 22000 |
| 2 | KTVGTVY | 585 | AAGACTGTGGGTACTGTTTAT | 586 | 45980 | 24000 | 21980 |
| 3 | KTVGTVY | 587 | AAAACCGTCGGCACAGTGTAC | 588 | 45181 | 24000 | 21181 |
| 4 | RSVGSVY | 589 | CGATCCGTCGGAAGCGTTTAC | 590 | 44994 | 22000 | 22994 |
| 5 | RYLGDAS | 591 | CGTTACTTAGGAGACGCCTCT | 592 | 40592 | 22245 | 18347 |
| 6 | WVLPSGG | 593 | TGGGTGCTACCATCTGGCGGC | 594 | 37597 | 20094 | 17504 |
| 7 | WVLPSGG | 595 | TGGGTTCTGCCTAGTGGTGGG | 596 | 34140 | 18433 | 15707 |
| 8 | VTVGSIY | 597 | GTAACAGTGGGCAGCATCTAC | 598 | 32968 | 19818 | 13150 |
| 9 | VRGSSIL | 599 | GTGCGTGGGTCGTCGATTCTT | 600 | 32330 | 19458 | 12872 |
| 10 | RYLGDAS | 601 | CGGTATTTGGGGGATGCTTCG | 602 | 32329 | 19921 | 12408 |
| 11 | VIQAMKL | 603 | GTGATTCAGGCTATGAAGTTG | 604 | 32127 | 17837 | 14290 |
| 12 | LTYGMAQ | 605 | CTGACTTATGGTATGGCTCAG | 606 | 31956 | 13152 | 18805 |
| 13 | LRIGLSQ | 607 | CTTCGGATTGGGCTGTCGCAG | 608 | 31710 | 13287 | 18423 |
| 14 | RYSGDAS | 609 | CGGTACTCAGGAGACGCTTCT | 610 | 31198 | 24000 | 7198 |
| 15 | RHHGDAA | 611 | CGGCATCATGGTGATGCGGCG | 612 | 30406 | 16501 | 13905 |
| 16 | VNYSVAL | 613 | GTGAACTACAGTGTCGCTCTA | 614 | 29969 | 19408 | 10561 |
| 17 | RHIADAS | 615 | CGTCATATTGCTGATGCTAGT | 616 | 29554 | 19310 | 10244 |
| 18 | RYLGDAT | 617 | CGGTATTTGGGGGATGCTACG | 618 | 29527 | 16761 | 12767 |
| 19 | QRVGFAQ | 619 | CAACGAGTCGGGTTCGCACAA | 620 | 29454 | 9203 | 20251 |
| 20 | RYSGDSV | 621 | AGGTACTCAGGCGACTCAGTC | 622 | 28960 | 17675 | 11286 |
| 21 | RHIADAS | 623 | AGACACATAGCGGACGCGTCG | 624 | 28595 | 15266 | 13329 |
| 22 | IAHGYST | 625 | ATTGCTCATGGGTATTCGACT | 626 | 28216 | 16364 | 11851 |
| 23 | VNYSVAL | 627 | GTTAATTATTCGGTGGCGCTT | 628 | 27811 | 19586 | 8225 |
| 24 | WTLESGH | 629 | TGGACCTTAGAAAGCGGGCAC | 630 | 27471 | 10055 | 17416 |
| 25 | RYSGDSV | 631 | CGTTATTCGGGGGATTCGGTT | 632 | 27410 | 14916 | 12493 |
| 26 | WTLESGH | 633 | TGGACTCTGGAGTCTGGTCAT | 634 | 27194 | 11189 | 16004 |
| 27 | VTVGSIY | 635 | GTTACTGTTGGGTCTATTTAT | 636 | 27069 | 16971 | 10098 |
| 28 | RYLGDAT | 637 | CGATACCTAGGTGACGCAACC | 638 | 26494 | 15312 | 11182 |
| 29 | RYSGDAS | 639 | AGGTATTCGGGTGATGCGAGT | 640 | 25873 | 20351 | 5522 |
| 30 | GDYSMIV | 641 | GGAGACTACTCTATGATAGTC | 642 | 24847 | 11475 | 13372 |
| 31 | GENSARW | 643 | GGGGAAAACTCTGCCAGATGG | 644 | 24447 | 13369 | 11078 |
| 32 | LAVGQKW | 645 | TTGGCGGTGGGGCAGAAGTGG | 646 | 24445 | 15404 | 9040 |
| 33 | SLDKPFK | 647 | AGTTTGGATAAGCCTTTTAAG | 648 | 24000 | 0 | 24000 |
| 34 | TLAVPFK | 649 | ACTTTGGCGGTGCCTTTTAAG | 650 | 24000 | 0 | 24000 |
| 35 | SLDKPFK | 651 | AGCTTAGACAAACCATTCAAA | 652 | 24000 | 0 | 24000 |
| 36 | RHHGDAA | 653 | AGACACCACGGGGACGCCGCA | 654 | 23998 | 13808 | 10190 |
| 37 | VKLGYSQ | 655 | GTGAAGCTTGGGTATTCGCAG | 656 | 23912 | 11914 | 11998 |
| 38 | GDYSMIV | 657 | GGGGATTATTCGATGATTGTG | 658 | 23621 | 12465 | 11156 |
| 39 | VRGSSIL | 659 | GTAAGAGGTTCCAGCATCCTA | 660 | 23580 | 16294 | 7286 |
| 40 | EAGSARW | 661 | GAAGCAGGTTCCGCTCGATGG | 662 | 23477 | 8257 | 15220 |
| 41 | EAGSARW | 663 | GAGGCGGGGAGTGCGCGGTGG | 664 | 22692 | 10133 | 12559 |
| 42 | QRVGFAQ | 665 | CAGAGGGTGGGTTTTGCGCAG | 666 | 22222 | 8003 | 14219 |
| 43 | WAISDGY | 667 | TGGGCAATCTCTGACGGCTAC | 668 | 21970 | 7625 | 14345 |
| 44 | LTYGMAQ | 669 | CTTACGTACGGGATGGCACAA | 670 | 21785 | 7103 | 14682 |
| 45 | RGPGLSQ | 671 | CGAGGCCCAGGCCTTAGCCAA | 672 | 21738 | 14881 | 6857 |
| 46 | SVSKPFL | 673 | AGTGTGAGTAAGCCTTTTTTG | 674 | 21352 | 0 | 21352 |
| 47 | LRIGLSQ | 675 | CTACGCATAGGCCTAAGCCAA | 676 | 19761 | 5023 | 14738 |
| 48 | RGPGLSQ | 677 | CGGGGTCCTGGGCTGTCTCAG | 678 | 19667 | 12307 | 7360 |
| 49 | IAHGYST | 679 | ATCGCCCACGGATACAGCACA | 680 | 19631 | 13047 | 6584 |
| 50 | RYVGESS | 681 | AGGTATGTGGGGGAGTCTTCG | 682 | 19630 | 8917 | 10713 |
| 51 | VIQAMKL | 683 | GTTATCCAAGCGATGAAACTA | 684 | 19442 | 12014 | 7428 |
| 52 | GPTMLFK | 685 | GGCCCAACAATGTTATTCAAA | 686 | 19036 | 0 | 19036 |
| 53 | GENSARW | 687 | GGTGAGAATAGTGCTCGGTGG | 688 | 16736 | 6244 | 10493 |
| 54 | LAVGQKW | 689 | CTCGCTGTCGGACAAAAATGG | 690 | 15886 | 9340 | 6546 |
| 55 | FTLTTPK | 691 | TTTACGTTGACGACGCCTAAG | 692 | 15607 | 254 | 15352 |
| 56 | EDLLRLR | 693 | GAGGATCTTTTGCGTCTTAGG | 694 | 14920 | 9436 | 5484 |
| 57 | LNYSVSL | 695 | CTGAACTACAGTGTATCCCTA | 696 | 14608 | 10933 | 3675 |
| 58 | WAISDGY | 697 | TGGGCGATTAGTGATGGGTAT | 698 | 14342 | 4386 | 9955 |
| 59 | GPTMLFK | 699 | GGTCCGACGATGTTGTTTAAG | 700 | 14140 | 0 | 14140 |
| 60 | RYVGESS | 701 | AGATACGTAGGTGAAAGTTCT | 702 | 13185 | 7591 | 5594 |
| 61 | PIIEHAV | 703 | CCGATTATTGAGCATGCGGTG | 704 | 12837 | 2394 | 10443 |
| 62 | SLSTPFR | 705 | TCTCTTTCTACGCCTTTTCGT | 706 | 12297 | 0 | 12297 |
| 63 | VKLGYSQ | 707 | GTTAAATTGGGTTACTCCCAA | 708 | 11170 | 6081 | 5089 |
| 64 | LNYSVSL | 709 | TTGAATTATTCTGTGAGTTTG | 710 | 11132 | 7243 | 3889 |
| 65 | LGTYELD | 711 | CTTGGGACTTATGAGCTTGAT | 712 | 11116 | 0 | 11116 |
| 66 | PIIEHAV | 713 | CCCATAATAGAACACGCAGTA | 714 | 11100 | 2000 | 9100 |
| 67 | RYISDSA | 715 | CGATACATAAGTGACTCCGCT | 716 | 10788 | 2084 | 8703 |
| 68 | WSTSSGF | 717 | TGGAGTACATCATCGGGATTC | 718 | 10614 | 628 | 9985 |
| 69 | WSLGSGH | 719 | TGGTCACTAGGAAGTGGTCAC | 720 | 10498 | 1235 | 9263 |
| 70 | WSQSSGY | 721 | TGGAGTCAGTCTAGTGGTTAT | 722 | 10258 | 728 | 9529 |
| 71 | EDLLRLR | 723 | GAAGACTTGCTGAGACTGCGA | 724 | 9754 | 6426 | 3328 |
| 72 | TEKLPFR | 725 | ACTGAGAAGCTGCCTTTTCGG | 726 | 9501 | 0 | 9501 |
| 73 | IMLGYST | 727 | ATTATGTTGGGGTATTCGACT | 728 | 9404 | 685 | 8718 |
| 74 | SLSTPFR | 729 | AGCCTCAGCACCCCCTTCCGC | 730 | 9222 | 0 | 9222 |
| 75 | ASNPGRW | 731 | GCGAGTAACCCTGGAAGGTGG | 732 | 9173 | 2435 | 6738 |
| 76 | WSLGSGH | 733 | TGGTCGTTGGGTTCTGGGCAT | 734 | 8761 | 0 | 8761 |
| 77 | NLIKPFL | 735 | AACCTTATAAAACCGTTCCTC | 736 | 8707 | 0 | 8707 |
| 78 | HVENWHI | 737 | CATGTGGAGAATTGGCATATT | 738 | 8680 | 634 | 8046 |
| 79 | WSQSSGY | 739 | TGGTCCCAAAGCTCTGGGTAC | 740 | 8560 | 590 | 7970 |
| 80 | WSTSSGF | 741 | TGGTCGACTAGTAGTGGTTTT | 742 | 8268 | 0 | 8268 |
| 81 | GKSPGVW | 743 | GGCAAATCCCCTGGAGTATGG | 744 | 7685 | 7236 | 448 |
| 82 | TEKLPFR | 745 | ACGGAAAAACTTCCGTTCAGG | 746 | 7240 | 0 | 7240 |
| 83 | SLVTSST | 747 | TCGCTTGTTACTTCTAGTACG | 748 | 6782 | 2782 | 4000 |
| 84 | LLYGYSS | 749 | CTTCTGTATGGTTATTCGAGT | 750 | 6779 | 334 | 6444 |
| 85 | VAGSSIL | 751 | GTTGCGGGTTCGTCGATTCTG | 752 | 6520 | 0 | 6520 |
| 86 | GLNERVA | 753 | GGTCTGAATGAGCGTGTGGCG | 754 | 6410 | 2000 | 4410 |
| 87 | LGTYELD | 755 | TTAGGCACATACGAATTGGAC | 756 | 6394 | 0 | 6394 |
| 88 | KNRRHSV | 757 | AAAAACCGTCGGCACAGTGTA | 758 | 6312 | 6190 | 122 |
| 89 | YTLSQGW | 759 | TATACTTTGTCGCAGGGTTGG | 760 | 6310 | 1912 | 4398 |
| 90 | SANPVVT | 761 | TCTGCGAATCCGGTTGTGACG | 762 | 5937 | 4778 | 1159 |
| 91 | VAGSSIL | 763 | GTAGCTGGGAGCAGCATCTTG | 764 | 5609 | 894 | 4716 |
| 92 | NLIKPFL | 765 | AATTTGATTAAGCCTTTTCTT | 766 | 5597 | 0 | 5597 |
| 93 | GGTSSGH | 767 | GGGGGTACGAGTAGTGGTCAT | 768 | 5462 | 4092 | 1370 |
| 94 | VLESNPR | 769 | GTGCTTGAGTCGAATCCGCGG | 770 | 5399 | 2617 | 2782 |
| 95 | WADSKDQ | 771 | TGGGCTGATAGTAAGGATCAG | 772 | 5374 | 2986 | 2388 |
| 96 | VDHGGVV | 773 | GTGGATCATGGTGGTGTGGTT | 774 | 5342 | 4000 | 1342 |
| 97 | ASTDSKT | 775 | GCTAGTACTGATTCTAAGACG | 776 | 5285 | 5285 | 0 |
| 98 | KGASVTL | 777 | AAGGGGGCTAGTGTTACGCTT | 778 | 5236 | 4000 | 1236 |
| 99 | SNVALTG | 779 | AGCAACGTTGCACTGACCGGC | 780 | 5235 | 2000 | 3235 |
| 100 | VIASNEH | 781 | GTGATTGCTTCTAATGAGCAT | 782 | 5109 | 3564 | 1545 |
| 101 | MSVGQSW | 783 | ATGTCGGTTGGGCAGTCGTGG | 784 | 5098 | 1046 | 4052 |
| 102 | LSNGQGP | 785 | TTGAGTAATGGTCAGGGTCCT | 786 | 5071 | 4076 | 996 |
| 103 | PVTDSKM | 787 | CCAGTAACTGACTCAAAAATG | 788 | 5068 | 1646 | 3422 |
| 104 | AADSSGR | 789 | GCGGCGGATAGTTCTGGGCGG | 790 | 4995 | 4520 | 475 |
| 105 | ANSHTNS | 791 | GCAAACAGTCACACCAACTCT | 792 | 4935 | 3542 | 1393 |
| 106 | VGANAVA | 793 | GTTGGTGCTAATGCTGTTGCT | 794 | 4872 | 3393 | 1479 |
| 107 | HVENWHI | 795 | CACGTTGAAAACTGGCACATC | 796 | 4865 | 0 | 4864 |
| 108 | KVDQSLA | 797 | AAAGTAGACCAATCACTTGCA | 798 | 4796 | 4796 | 0 |
| 109 | SGEALRL | 799 | TCAGGTGAAGCACTACGGCTA | 800 | 4788 | 971 | 3817 |
| 110 | VPSSTER | 801 | GTTCCGAGTTCTACTGAGCGG | 802 | 4782 | 2664 | 2118 |
| 111 | VVQVNGR | 803 | GTCGTGCAAGTGAACGGACGC | 804 | 4738 | 4415 | 323 |
| 112 | ASNPGRW | 805 | GCTTCGAATCCGGGTCGGTGG | 806 | 4729 | 3678 | 1051 |
| 113 | HNGQVGV | 807 | CACAACGGACAAGTGGGAGTC | 808 | 4705 | 2736 | 1968 |
| 114 | SLAITER | 809 | AGTTTGGCGATTACTGAGCGG | 810 | 4664 | 4000 | 664 |
| 115 | SSAGTSA | 811 | AGTTCGGCGGGTACTTCGGCG | 812 | 4632 | 2000 | 2632 |
| 116 | SVDNRDS | 813 | AGTGTGGACAACAGAGACAGT | 814 | 4614 | 2614 | 2000 |
| 117 | VGQTTTL | 815 | GTTGGCCAAACCACAACATTG | 816 | 4612 | 4428 | 184 |
| 118 | GQVQMTS | 817 | GGTCAGGTGCAGATGACTTCT | 818 | 4572 | 3903 | 669 |
| 119 | GMHVVQA | 819 | GGAATGCACGTCGTCCAAGCA | 820 | 4554 | 0 | 4554 |
| 120 | VGVPLGR | 821 | GTTGGGGTTCCCCTAGGGAGA | 822 | 4548 | 0 | 4548 |
| 121 | KESTLST | 823 | AAGGAGTCTACTCTGAGTACG | 824 | 4541 | 1579 | 2962 |
| 122 | RETVGST | 825 | CGCGAAACCGTAGGCAGTACT | 826 | 4537 | 537 | 4000 |
| 123 | GNGSTSL | 827 | GGTAACGGAAGCACATCGCTA | 828 | 4511 | 438 | 4073 |
| 124 | VDSTISI | 829 | GGTAACGGAAGCACATCGCTA | 830 | 4497 | 4391 | 106 |
| 125 | ASTATIR | 831 | GCGTCTACTGCTACTATTCGG | 832 | 4484 | 4205 | 279 |
| 126 | RSHDSET | 833 | AGGAGTCATGATTCTGAGACT | 834 | 4473 | 0 | 4473 |
| 127 | FGSQMGA | 835 | TTTGGGTCTCAGATGGGGGCG | 836 | 4454 | 4303 | 151 |
| 128 | SVTDVKL | 837 | TCTGTTACTGATGTTAAGCTT | 838 | 4449 | 2449 | 2000 |
| 129 | SHVSDSK | 839 | AGCCACGTATCCGACTCCAAA | 840 | 4446 | 0 | 4446 |
| 130 | KLAPDGT | 841 | AAACTTGCTCCCGACGGAACG | 842 | 4429 | 4000 | 429 |
| 131 | YLVGYQM | 843 | TACCTAGTGGGGTACCAAATG | 844 | 4419 | 899 | 3520 |
| 132 | QDKSTYK | 845 | CAGGATAAGTCGACGTATAAG | 846 | 4396 | 2495 | 1901 |
| 133 | PGGESRG | 847 | CCCGGAGGCGAAAGCCGAGGC | 848 | 4395 | 2000 | 2395 |
| 134 | LTGSVQL | 849 | CTTACGGGTTCGGTTCAGCTT | 850 | 4386 | 1851 | 2535 |
| 135 | SGETLRL | 851 | AGTGGCGAAACCCTACGTCTC | 852 | 4385 | 4000 | 385 |
| 136 | PSVSTLS | 853 | CCTAGTGTTTCTACGCTTAGT | 854 | 4380 | 2380 | 2000 |
| 137 | PGTVNTH | 855 | CCTGGTACTGTTAATACGCAT | 856 | 4354 | 354 | 4000 |
| 138 | PSQGMTS | 857 | CCATCCCAAGGAATGACATCC | 858 | 4340 | 4163 | 177 |
| 139 | AGVLNTL | 859 | GCGGGGGTGCTGAATACTTTG | 860 | 4329 | 2000 | 2329 |
| 140 | KNHGVDP | 861 | AAAAACCACGGAGTAGACCCC | 862 | 4315 | 4017 | 298 |
| 141 | GLMTNAK | 863 | GGGCTGATGACGAATGCGAAG | 864 | 4303 | 2000 | 2303 |
| 142 | MNGVHVL | 865 | ATGAACGGAGTCCACGTACTT | 866 | 4301 | 1871 | 2430 |
| 143 | TDVHSTS | 867 | ACGGATGTGCATTCGACTTCG | 868 | 4296 | 2000 | 2296 |
| 144 | IMLGYST | 869 | ATCATGCTGGGTTACTCTACG | 870 | 4268 | 284 | 3984 |
| 145 | PAEHYQA | 871 | CCGGCTGAGCATTATCAGGCT | 872 | 4259 | 4259 | 0 |
| 146 | GQTLAES | 873 | GGGCAAACATTAGCGGAATCG | 874 | 4245 | 956 | 3289 |
| 147 | SSVQGIL | 875 | TCGAGTGTTCAGGGGATTCTG | 876 | 4232 | 2232 | 2000 |
| 148 | ALSQIEV | 877 | GCGCTGTCCCAAATAGAAGTC | 878 | 4230 | 686 | 3544 |
| 149 | VTTVTPV | 879 | GTCACGACTGTGACCCCCGTT | 880 | 4223 | 1622 | 2601 |
| 150 | ATVTGAD | 881 | GCTACGGTGACCGGAGCAGAC | 882 | 4207 | 4207 | 0 |
| 151 | VSGGDYS | 883 | GTCTCTGGAGGCGACTACTCA | 884 | 4202 | 2639 | 1563 |
| 152 | KSQSEDV | 885 | AAGAGTCAGTCTGAGGATGTG | 886 | 4202 | 2000 | 2202 |
| 153 | LLYGYSS | 887 | CTCCTCTACGGATACTCTTCA | 888 | 4198 | 0 | 4198 |
| 154 | MVQSGLT | 889 | ATGGTTCAGTCGGGGTTGACG | 890 | 4198 | 2000 | 2198 |
| 155 | EQYLGSP | 891 | GAGCAGTATCTGGGTTCTCCG | 892 | 4196 | 2000 | 2196 |
| 156 | SGANLSN | 893 | TCGGGGGCTAACCTCTCGAAC | 894 | 4181 | 3761 | 420 |
| 157 | ALVQNGV | 895 | GCTTTGGTGCAGAATGGTGTT | 896 | 4165 | 2724 | 1441 |
| 158 | SDQQKVW | 897 | AGTGATCAGCAGAAGGTTTGG | 898 | 4165 | 3760 | 405 |
| 159 | NGSDTPK | 899 | AACGGAAGCGACACACCGAAA | 900 | 4163 | 449 | 3714 |
| 160 | SDLTSYV | 901 | TCCGACCTCACCAGTTACGTT | 902 | 4160 | 2553 | 1607 |
| 161 | GAADRQI | 903 | GGTGCGGCTGATAGGCAGATT | 904 | 4143 | 0 | 4143 |
| 162 | AVGHVSG | 905 | GCCGTAGGACACGTTTCTGGT | 906 | 4143 | 2000 | 2143 |
| 163 | RYISDSA | 907 | AGGTATATTTCGGATTCTGCG | 908 | 4142 | 3503 | 639 |
| 164 | AQGTISR | 909 | GCGCAGGGGACGATTTCGCGT | 910 | 4136 | 2550 | 1586 |
| 165 | MANMLSD | 911 | ATGGCGAACATGTTATCTGAC | 912 | 4132 | 236 | 3896 |
| 166 | VMRDKDE | 913 | GTGATGCGTGATAAGGATGAG | 914 | 4126 | 2000 | 2126 |
| 167 | GGKGEGP | 915 | GGTGGGAAGGGTGAGGGTCCG | 916 | 4101 | 1032 | 3069 |
| 168 | SDVVVTH | 917 | TCTGATGTTGTTGTTACTCAT | 918 | 4088 | 3762 | 326 |
| 169 | GVLTTVT | 919 | GGGGTTCTTACTACGGTGACT | 920 | 4086 | 0 | 4086 |
| 170 | NDGPREQ | 921 | AATGATGGTCCGAGGGAGCAG | 922 | 4043 | 3837 | 206 |
| 171 | PTQGVSM | 923 | CCAACCCAAGGAGTTTCGATG | 924 | 4035 | 1735 | 2300 |
| 172 | NMGVVQL | 925 | AACATGGGCGTCGTGCAATTG | 926 | 4032 | 0 | 4032 |
| 173 | AGGGDPR | 927 | GCGGGGGGTGGGGATCCGAGG | 928 | 4031 | 2780 | 1250 |
| 174 | AGVVNAL | 929 | GCGGGGGTGGTGAATGCTTTG | 930 | 4015 | 1870 | 2145 |
| 175 | ANPVGNV | 931 | GCGAATCCTGTTGGGAATGTT | 932 | 4000 | 2483 | 1517 |
| 176 | VQGTQTG | 933 | GTGCAGGGTACGCAGACTGGT | 934 | 4000 | 2000 | 2000 |
| 177 | SPGFSIA | 935 | TCTCCTGGATTCAGCATCGCT | 936 | 4000 | 2000 | 2000 |
| 178 | AIERLTV | 937 | GCAATCGAAAGACTAACCGTT | 938 | 4000 | 2000 | 2000 |
| 179 | TDKQNAF | 939 | ACGGACAAACAAAACGCATTC | 940 | 4000 | 2000 | 2000 |
| 180 | LADSKDR | 941 | CTGGCAGACTCGAAAGACAGG | 942 | 4000 | 2000 | 2000 |
| 181 | VHSTGEW | 943 | GTACACAGCACAGGCGAATGG | 944 | 4000 | 2000 | 2000 |
| 182 | LHNALAV | 945 | CTGCATAATGCTCTGGCTGTT | 946 | 4000 | 4000 | 0 |
| 183 | IGLDPKA | 947 | ATTGGTTTGGATCCGAAGGCG | 948 | 3982 | 3549 | 433 |
| 184 | VMASTGP | 949 | GTTATGGCTTCGACTGGTCCT | 950 | 3980 | 3654 | 326 |
| 185 | SVPGTVS | 951 | AGTGTGCCGGGGACTGTGTCT | 952 | 3970 | 2804 | 1166 |
| 186 | MLSNGQV | 953 | ATGCTGTCTAATGGGCAGGTT | 954 | 3965 | 3965 | 0 |
| 187 | ASTATLR | 955 | GCGTCTACTGCTACTCTTCGG | 956 | 3963 | 2082 | 1880 |
| 188 | PGEHYQG | 957 | CCGGGTGAGCATTATCAGGGT | 958 | 3963 | 1963 | 2000 |
| 189 | QVTDNKT | 959 | CAGGTGACTGATAATAAGACT | 960 | 3957 | 2201 | 1756 |
| 190 | NGLQVSI | 961 | AACGGACTACAAGTGTCTATC | 962 | 3956 | 3956 | 0 |
| 191 | SHPGNEL | 963 | AGCCACCCCGGCAACGAACTC | 964 | 3952 | 3764 | 188 |
| 192 | VSLNGGH | 965 | GTGTCGCTTAATGGGGGGCAT | 966 | 3950 | 1950 | 2000 |
| 193 | MVASSID | 967 | ATGGTGGCTTCATCCATAGAC | 968 | 3938 | 2000 | 1938 |
| 194 | MGVNTTI | 969 | ATGGGGGTGAATACGACTATT | 970 | 3936 | 3388 | 548 |
| 195 | MNGGHLM | 971 | ATGAATGGGGGTCATCTTATG | 972 | 3934 | 3406 | 528 |
| 196 | LGSDGRT | 973 | CTTGGCTCAGACGGCCGAACC | 974 | 3931 | 1384 | 2547 |
| 197 | RVDTPQL | 975 | CGAGTCGACACACCACAATTG | 976 | 3926 | 1566 | 2360 |
| 198 | TEQAKLS | 977 | ACTGAACAAGCCAAACTATCT | 978 | 3925 | 1925 | 2000 |
| 199 | IGTNSTY | 979 | ATTGGTACGAATAGTACGTAT | 980 | 3903 | 3847 | 56 |
| 200 | LSESANR | 981 | TTGTCGGAGAGTGCGAATCGT | 982 | 3891 | 2810 | 1082 |
| 201 | GQSSNQH | 983 | GGGCAGTCGTCTAATCAGCAT | 984 | 3855 | 1478 | 2377 |
| 202 | MRSEQTT | 985 | ATGCGTAGTGAGCAGACGACG | 986 | 3854 | 2000 | 1854 |
| 203 | AGISTQT | 987 | GCGGGGATTAGTACTCAGACG | 988 | 3835 | 1783 | 2052 |
| 204 | TGESNVG | 989 | ACTGGGGAGAGTAATGTTGGT | 990 | 3820 | 2000 | 1820 |
| 205 | ALANVSN | 991 | GCGCTTGCCAACGTTTCCAAC | 992 | 3800 | 0 | 3800 |
| 206 | SFGMVVD | 993 | TCGTTCGGCATGGTAGTCGAC | 994 | 3800 | 0 | 3800 |
| 207 | TMSHAEL | 995 | ACCATGTCGCACGCAGAATTA | 996 | 3792 | 1792 | 2000 |
| 208 | LSNMVSA | 997 | TTGAGTAATATGGTGAGTGCT | 998 | 3778 | 450 | 3328 |
| 209 | HGTLVSR | 999 | CATGGGACTTTGGTGTCTCGG | 1000 | 3763 | 1763 | 2000 |
| 210 | VAVTGAI | 1001 | GTTGCGGTGACTGGTGCTATT | 1002 | 3756 | 1952 | 1803 |
| 211 | IMVDAHA | 1003 | ATTATGGTTGATGCTCATGCG | 1004 | 3753 | 3753 | 0 |
| 212 | TPTLPFI | 1005 | ACGCCTACGTTGCCTTTTATT | 1006 | 3751 | 0 | 3751 |
| 213 | GIAGLGI | 1007 | GGTATTGCTGGGCTTGGGATT | 1008 | 3750 | 1438 | 2312 |
| 214 | SSLPDKT | 1009 | TCATCCCTACCGGACAAAACC | 1010 | 3746 | 0 | 3746 |
| 215 | QSQTALR | 1011 | CAATCCCAAACAGCATTGCGA | 1012 | 3744 | 2362 | 1382 |
| 216 | AVGNELL | 1013 | GCCGTTGGCAACGAACTGCTG | 1014 | 3744 | 1744 | 2000 |
| 217 | GSGAGVA | 1015 | GGGAGCGGCGCCGGTGTAGCC | 1016 | 3744 | 3663 | 81 |
| 218 | ESGLINV | 1017 | GAGTCGGGTCTGATTAATGTG | 1018 | 3732 | 5 | 3727 |
| 219 | PNAGFDR | 1019 | CCGAACGCGGGGTTCGACCGT | 1020 | 3720 | 1720 | 2000 |
| 220 | LNQGLGD | 1021 | CTGAATCAGGGGTTGGGGGAT | 1022 | 3716 | 2002 | 1714 |
| 221 | ALASVGV | 1023 | GCGTTGGCATCCGTGGGTGTC | 1024 | 3715 | 1489 | 2226 |
| 222 | SEGPSRY | 1025 | TCGGAGGGTCCTTCGCGTTAT | 1026 | 3710 | 2201 | 1509 |
| 223 | SYGDGGV | 1027 | TCGTATGGTGATGGTGGTGTT | 1028 | 3708 | 2913 | 795 |
| 224 | LGHNSGV | 1029 | TTGGGGCATAATTCTGGTGTT | 1030 | 3696 | 1406 | 2290 |
| 225 | SSPNVGP | 1031 | TCTTCTCCGAACGTCGGTCCT | 1032 | 3688 | 2600 | 1088 |
| 226 | VSGTSTH | 1033 | GTTTCGGGTACTTCTACGCAT | 1034 | 3687 | 3471 | 216 |
| 227 | SEGGNNR | 1035 | AGTGAGGGTGGGAATAATCGG | 1036 | 3680 | 2379 | 1302 |
| 228 | SGASLSN | 1037 | TCCGGAGCATCCCTTTCCAAC | 1038 | 3672 | 0 | 3672 |
| 229 | SNGVPSS | 1039 | AGCAACGGAGTACCGTCATCG | 1040 | 3659 | 1618 | 2041 |
| 230 | ESGTHLS | 1041 | GAGTCTGGGACTCATTTGTCG | 1042 | 3655 | 2745 | 909 |
| 231 | VTVQVQR | 1043 | GTTACGGTGCAGGTGCAGAGG | 1044 | 3652 | 3652 | 0 |
| 232 | ASESTPR | 1045 | GCTAGTGAGTCTACGCCGCGT | 1046 | 3651 | 1665 | 1986 |
| 233 | LVTFRAD | 1047 | CTTGTTACTTTTCGTGCGGAT | 1048 | 3646 | 2633 | 1013 |
| 234 | LTQMSNK | 1049 | CTGACTCAGATGAGTAATAAG | 1050 | 3640 | 2963 | 677 |
| 235 | AVEGSRL | 1051 | GCGGTGGAGGGTTCGAGGCTG | 1052 | 3635 | 2008 | 1627 |
| 236 | PNERINV | 1053 | CCGAATGAGAGGATTAATGTG | 1054 | 3626 | 2003 | 1623 |
| 237 | GDHDRGS | 1055 | GGAGACCACGACAGGGGCTCG | 1056 | 3617 | 3617 | 0 |
| 238 | RHQVSES | 1057 | CGTCATCAGGTTAGTGAGAGT | 1058 | 3610 | 2863 | 747 |
| 239 | LDGLNLH | 1059 | CTAGACGGCTTGAACCTCCAC | 1060 | 3604 | 1330 | 2274 |
| 240 | PGNGTLV | 1061 | CCGGGGAATGGGACGTTGGTT | 1062 | 3598 | 2238 | 1360 |
| 241 | DSYGGNA | 1063 | GACTCGTACGGGGGGAACGCC | 1064 | 3596 | 3570 | 26 |
| 242 | LHKGSES | 1065 | CTTCATAAGGGTAGTGAGAGT | 1066 | 3593 | 2662 | 931 |
| 243 | SVDIVKL | 1067 | TCGGTTGATATTGTGAAGCTT | 1068 | 3590 | 626 | 2964 |
| 244 | HTLSTGV | 1069 | CATACGCTGAGTACTGGGGTG | 1070 | 3588 | 2000 | 1588 |
| 245 | SQINSGS | 1071 | TCCCAAATAAACTCTGGCAGC | 1072 | 3583 | 3046 | 537 |
| 246 | ALSGLDK | 1073 | GCCCTGAGTGGGCTAGACAAA | 1074 | 3582 | 3582 | 0 |
| 247 | RNSESEA | 1075 | CGAAACAGCGAATCGGAAGCG | 1076 | 3576 | 739 | 2837 |
| 248 | PNERHTL | 1077 | CCTAACGAACGCCACACCTTG | 1078 | 3575 | 2000 | 1575 |
| 249 | VNAGLGI | 1079 | GTAAACGCCGGCTTGGGCATC | 1080 | 3571 | 2987 | 584 |
| 250 | PASGALT | 1081 | CCGGCTTCGGGTGCTCTTACT | 1082 | 3567 | 1259 | 2308 |
| 251 | SAMVTSP | 1083 | TCAGCCATGGTTACCTCGCCA | 1084 | 3564 | 1057 | 2507 |
| 252 | DSHVSGK | 1085 | GACTCACACGTCAGTGGAAAA | 1086 | 3556 | 2082 | 1474 |
| 253 | SPQGALA | 1087 | TCGCCGCAGGGGGCTCTTGCT | 1088 | 3553 | 0 | 3553 |
| 254 | GDNPAVA | 1089 | GGTGATAATCCTGCGGTGGCT | 1090 | 3547 | 3363 | 184 |
| 255 | KEIHVSV | 1091 | AAAGAAATCCACGTTTCTGTG | 1092 | 3543 | 3345 | 197 |
| 256 | VTTVSTV | 1093 | GTGACTACGGTTTCTACTGTG | 1094 | 3542 | 1419 | 2124 |
| 257 | WTDGVSR | 1095 | TGGACTGATGGGGTGTCGCGG | 1096 | 3538 | 867 | 2671 |
| 258 | AADSSAR | 1097 | GCGGCGGATAGTTCTGCGCGG | 1098 | 3535 | 3428 | 107 |
| 259 | LDNRTMK | 1099 | CTTGATAATCGTACTATGAAG | 1100 | 3531 | 4 | 3527 |
| 260 | DVADSKR | 1101 | GATGTTGCTGATTCTAAGCGT | 1102 | 3530 | 2001 | 1529 |
| 261 | SVGGTIH | 1103 | TCGGTTGGTGGTACGATTCAT | 1104 | 3522 | 2000 | 1522 |
| 262 | PLTAGVS | 1105 | CCTCTGACGGCGGGGGTGTCG | 1106 | 3520 | 1181 | 2339 |
| 263 | AADISVR | 1107 | GCAGCAGACATATCAGTCCGC | 1108 | 3518 | 3518 | 0 |
| 264 | YADSHTD | 1109 | TACGCCGACAGCCACACAGAC | 1110 | 3516 | 3516 | 0 |
| 265 | VDVNLTR | 1111 | GTCGACGTAAACTTAACAAGA | 1112 | 3512 | 1664 | 1848 |
| 266 | AIAEYQV | 1113 | GCGATTGCGGAGTATCAGGTG | 1114 | 3497 | 2231 | 1266 |
| 267 | SRVDGSG | 1115 | TCGCGTGTTGATGGTTCGGGT | 1116 | 3492 | 1573 | 1919 |
| 268 | HGDGVRV | 1117 | CACGGGGACGGAGTACGCGTC | 1118 | 3482 | 3345 | 137 |
| 269 | HESRDHS | 1119 | CACGAATCGAGAGACCACAGT | 1120 | 3477 | 2000 | 1477 |
| 270 | RYEQNTP | 1121 | AGGTACGAACAAAACACTCCC | 1122 | 3475 | 3022 | 453 |
| 271 | ALASTQT | 1123 | GCGTTGGCGAGTACTCAGACG | 1124 | 3475 | 2221 | 1254 |
| 272 | FSSERLP | 1125 | TTTTCGTCTGAGCGGCTTCCG | 1126 | 3467 | 3467 | 0 |
| 273 | TTTHEGV | 1127 | ACGACGACTCATGAGGGGGTG | 1128 | 3463 | 2359 | 1104 |
| 274 | RMDSAQL | 1129 | AGGATGGATTCGGCGCAGCTT | 1130 | 3450 | 0 | 3450 |
| 275 | SHGPDSK | 1131 | TCTCATGGTCCTGATTCGAAG | 1132 | 3448 | 2653 | 795 |
| 276 | SVATGVL | 1133 | TCGGTTGCGACGGGGGTTCTG | 1134 | 3447 | 3059 | 388 |
| 277 | LLASGAK | 1135 | CTGCTTGCGAGTGGGGCTAAG | 1136 | 3447 | 1315 | 2132 |
| 278 | ASLGAYS | 1137 | GCGTCGCTTGGGGCGTATTCG | 1138 | 3445 | 2442 | 1003 |
| 279 | KELLVSA | 1139 | AAAGAACTCTTAGTAAGTGCA | 1140 | 3440 | 425 | 3015 |
| 280 | SLGVAVA | 1141 | AGTTTAGGTGTCGCCGTCGCC | 1142 | 3440 | 2000 | 1440 |
| 281 | VSGSISK | 1143 | GTTTCGGGGAGTATTTCTAAG | 1144 | 3429 | 3108 | 322 |
| 282 | TTSGQTM | 1145 | ACCACTTCCGGTCAAACAATG | 1146 | 3427 | 3123 | 304 |
| 283 | QTVGPLN | 1147 | CAAACAGTAGGACCGTTAAAC | 1148 | 3410 | 1308 | 2102 |
| 284 | VNGNNTY | 1149 | GTAAACGGCAACAACACCTAC | 1150 | 3409 | 3122 | 287 |
| 285 | VAEGGGV | 1151 | GTAGCCGAAGGCGGTGGCGTC | 1152 | 3408 | 3139 | 269 |
| 286 | GSGENVR | 1153 | GGCTCTGGCGAAAACGTAAGG | 1154 | 3408 | 2977 | 431 |
| 287 | AADSSMR | 1155 | GCGGCGGATAGTTCTATGCGG | 1156 | 3406 | 3189 | 217 |
| 288 | LDGLNLH | 1157 | CTGGATGGGCTGAATCTTCAT | 1158 | 3402 | 582 | 2820 |
| 289 | MAGALGP | 1159 | ATGGCAGGTGCACTGGGTCCC | 1160 | 3400 | 2501 | 898 |
| 290 | GLNEHGA | 1161 | GGTCTGAATGAGCATGGGGCG | 1162 | 3390 | 2628 | 762 |
| 291 | LLSSENR | 1163 | CTCTTGTCTTCTGAAAACCGG | 1164 | 3389 | 2323 | 1066 |
| 292 | RDVSGHI | 1165 | AGGGATGTGTCGGGGCATATT | 1166 | 3383 | 3383 | 0 |
| 293 | GDQAMVN | 1167 | GGGGATCAGGCTATGGTTAAT | 1168 | 3380 | 0 | 3380 |
| 294 | AHVDVKV | 1169 | GCCCACGTAGACGTAAAAGTT | 1170 | 3379 | 537 | 2842 |
| 295 | ALANSER | 1171 | GCTTTGGCGAATTCTGAGCGG | 1172 | 3378 | 1661 | 1717 |
| 296 | KGSDTTI | 1173 | AAGGGTTCTGATACTACTATT | 1174 | 3378 | 2686 | 692 |
| 297 | VAQGSVV | 1175 | GTGGCTCAGGGGTCGGTTGTT | 1176 | 3371 | 2834 | 537 |
| 298 | GFEDGAR | 1177 | GGCTTCGAAGACGGTGCTCGA | 1178 | 3360 | 1746 | 1614 |
| 299 | QADNHVR | 1179 | CAGGCGGATAATCATGTTAGG | 1180 | 3357 | 2683 | 674 |
| 300 | RHADSTV | 1181 | CGTCATGCTGATTCTACGGTT | 1182 | 3355 | 3217 | 138 |
| 301 | PMSQGEL | 1183 | CCGATGAGTCAGGGGGAGTTG | 1184 | 3354 | 1354 | 2000 |
| 302 | GNSGGHV | 1185 | GGGAATAGTGGGGGTCATGTT | 1186 | 3350 | 2574 | 776 |
| 303 | RNQAEEM | 1187 | AGAAACCAAGCTGAAGAAATG | 1188 | 3348 | 123 | 3225 |
| 304 | LLSSENR | 1189 | CTTCTGTCGTCGGAGAATAGG | 1190 | 3346 | 514 | 2831 |
| 305 | GFEGGTR | 1191 | GGGTTCGAAGGCGGCACTCGA | 1192 | 3343 | 2297 | 1046 |
| 306 | GGGSESY | 1193 | GGAGGGGGGTCAGAATCATAC | 1194 | 3337 | 2984 | 353 |
| 307 | EAASAIS | 1195 | GAAGCGGCATCAGCCATATCC | 1196 | 3329 | 1054 | 2274 |
| 308 | LTTPIEL | 1197 | TTGACGACTCCGATTGAGTTG | 1198 | 3328 | 1036 | 2292 |
| 309 | RTMSVML | 1199 | CGCACCATGTCTGTCATGCTG | 1200 | 3326 | 1491 | 1835 |
| 310 | GIHETRA | 1201 | GGCATCCACGAAACACGGGCA | 1202 | 3320 | 2000 | 1320 |
| 311 | SEGHSSY | 1203 | TCGGAGGGTCATTCGAGTTAT | 1204 | 3317 | 2459 | 858 |
| 312 | SVSDVKH | 1205 | AGTGTCTCGGACGTCAAACAC | 1206 | 3308 | 3093 | 215 |
| 313 | LSVSQSA | 1207 | CTCTCCGTCAGTCAATCTGCT | 1208 | 3295 | 1511 | 1784 |
| 314 | VVKEYES | 1209 | GTAGTCAAAGAATACGAAAGC | 1210 | 3291 | 859 | 2432 |
| 315 | RVGAEGT | 1211 | CGGGTTGGGGCGGAGGGGACG | 1212 | 3281 | 3281 | 0 |
| 316 | QAGLGVI | 1213 | CAGGCTGGTCTTGGTGTTATT | 1214 | 3278 | 0 | 3278 |
| 317 | RVHSTDT | 1215 | CGAGTCCACTCGACCGACACG | 1216 | 3274 | 1079 | 2195 |
| 318 | VATESAF | 1217 | GTGGCAACCGAATCAGCATTC | 1218 | 3270 | 3270 | 0 |
| 319 | SNGAGYL | 1219 | TCGAATGGGGGGGGTTATCTT | 1220 | 3269 | 306 | 2963 |
| 320 | VGEGNKF | 1221 | GTGGGTGAGGGGAATAAGTTT | 1222 | 3268 | 816 | 2451 |
| 321 | DVRGSVI | 1223 | GATGTGAGGGGTTCGGTTATT | 1224 | 3263 | 1137 | 2126 |
| 322 | PLDGQGK | 1225 | CCGCTAGACGGCCAAGGCAAA | 1226 | 3255 | 3255 | 0 |
| 323 | ASVSSQL | 1227 | GCCAGCGTCAGCTCACAACTC | 1228 | 3254 | 1140 | 2114 |
| 324 | LAKEESH | 1229 | CTTGCTAAGGAGGAGTCGCAT | 1230 | 3252 | 704 | 2548 |
| 325 | FTHGTGT | 1231 | TTCACGCACGGCACTGGGACG | 1232 | 3241 | 2000 | 1241 |
| 326 | ASVSSQS | 1233 | GCCTCGGTCTCGAGTCAATCA | 1234 | 3232 | 3232 | 0 |
| 327 | GVADNVK | 1235 | GGTGTCGCAGACAACGTCAAA | 1236 | 3226 | 2673 | 553 |
| 328 | SAGVPGV | 1237 | TCGGCTGGAGTACCTGGAGTC | 1238 | 3223 | 1005 | 2218 |
| 329 | SDSTVVG | 1239 | TCTGATAGTACTGTTGTGGGG | 1240 | 3214 | 1119 | 2095 |
| 330 | FAGIAQA | 1241 | TTTGCGGGGATTGCGCAGGCG | 1242 | 3209 | 0 | 3209 |
| 331 | QSDLGRV | 1243 | CAGTCGGATCTTGGGAGGGTG | 1244 | 3209 | 3125 | 85 |
| 332 | PLQNNPH | 1245 | CCGCTTCAGAATAATCCGCAT | 1246 | 3204 | 1946 | 1258 |
| 333 | PGTNSFS | 1247 | CCCGGAACCAACAGTTTCTCT | 1248 | 3201 | 647 | 2554 |
| 334 | QEQGTST | 1249 | CAAGAACAAGGCACTTCGACG | 1250 | 3197 | 2891 | 306 |
| 335 | RSEVNGV | 1251 | CGTTCAGAAGTAAACGGTGTC | 1252 | 3196 | 3006 | 190 |
| 336 | LTDKMTS | 1253 | TTGACTGATAAGATGACGTCG | 1254 | 3194 | 1064 | 2130 |
| 337 | GGTISGP | 1255 | GGGGGTACGATTAGTGGTCCT | 1256 | 3190 | 3190 | 0 |
| 338 | ADGKGAI | 1257 | GCGGATGGGAAGGGTGCGATT | 1258 | 3188 | 3080 | 108 |
| 339 | RTGDTIS | 1259 | CGGACGGGCGACACAATCAGT | 1260 | 3185 | 3095 | 90 |
| 340 | PMSPGVA | 1261 | CCGATGTCTCCGGGGGTGGCT | 1262 | 3178 | 3178 | 0 |
| 341 | SVMTDRP | 1263 | TCGGTGATGACCGACAGACCT | 1264 | 3172 | 869 | 2303 |
| 342 | PTEGTLR | 1265 | CCTACTGAGGGGACGCTTCGG | 1266 | 3171 | 894 | 2278 |
| 343 | ASGTGMT | 1267 | GCCTCAGGGACCGGCATGACG | 1268 | 3171 | 1171 | 2000 |
| 344 | SANPVAR | 1269 | AGCGCTAACCCCGTAGCTCGG | 1270 | 3170 | 1988 | 1182 |
| 345 | AAGVNLN | 1271 | GCGGCGGGTGTTAATCTGAAT | 1272 | 3170 | 1915 | 1255 |
| 346 | TNQVITH | 1273 | ACAAACCAAGTAATAACTCAC | 1274 | 3169 | 2316 | 853 |
| 347 | PLINGLV | 1275 | CCTTTGATTAATGGGCTGGTG | 1276 | 3168 | 2731 | 437 |
| 348 | KSHSENN | 1277 | AAGTCGCATTCGGAGAATAAT | 1278 | 3168 | 3055 | 114 |
| 349 | MNGGHVK | 1279 | ATGAATGGGGGTCATGTTAAG | 1280 | 3165 | 1765 | 1400 |
| 350 | LAGTLVQ | 1281 | TTGGCAGGAACCCTAGTACAA | 1282 | 3163 | 2352 | 811 |
| 351 | PLKGGGE | 1283 | CCTCTGAAGGGTGGTGGGGAG | 1284 | 3161 | 3159 | 2 |
| 352 | VVNSSSS | 1285 | GTAGTGAACTCCTCTTCTTCC | 1286 | 3161 | 3161 | 0 |
| 353 | SKADAYS | 1287 | TCGAAGGCTGATGCTTATAGT | 1288 | 3160 | 3160 | 0 |
| 354 | MIGDVSP | 1289 | ATGATCGGAGACGTAAGTCCT | 1290 | 3159 | 3159 | 0 |
| 355 | LDSSRFH | 1291 | CTGGATAGTTCGCGTTTTCAT | 1292 | 3156 | 2645 | 512 |
| 356 | GLMSNAK | 1293 | GGACTCATGAGTAACGCAAAA | 1294 | 3156 | 1176 | 1981 |
| 357 | PSVGMAT | 1295 | CCTTCAGTTGGCATGGCGACT | 1296 | 3154 | 2400 | 755 |
| 358 | SSEGRNV | 1297 | AGTAGTGAGGGTCGTAATGTG | 1298 | 3152 | 2545 | 608 |
| 359 | LQEQLAG | 1299 | CTTCAGGAGCAGCTTGCTGGG | 1300 | 3152 | 930 | 2222 |
| 360 | VGVPLGR | 1301 | GTGGGTGTGCCGCTTGGTCGG | 1302 | 3149 | 0 | 3149 |
| 361 | AGASAEA | 1303 | GCTGGGGCTAGTGCTGAGGCG | 1304 | 3145 | 0 | 3145 |
| 362 | VNSSENK | 1305 | GTGAACAGCTCCGAAAACAAA | 1306 | 3145 | 1540 | 1605 |
| 363 | INGRNDI | 1307 | ATAAACGGCCGGAACGACATC | 1308 | 3143 | 2564 | 579 |
| 364 | VMASTGP | 1309 | GTAATGGCGTCAACAGGACCG | 1310 | 3140 | 1140 | 2000 |
| 365 | AASEVYV | 1311 | GCTGCTTCTGAGGTTTATGTT | 1312 | 3137 | 2581 | 556 |
| 366 | RNNVDST | 1313 | CGCAACAACGTAGACAGTACT | 1314 | 3136 | 2822 | 314 |
| 367 | LDASKLV | 1315 | CTCGACGCATCCAAATTGGTT | 1316 | 3130 | 996 | 2134 |
| 368 | AGDSSVR | 1317 | GCTGGTGACTCAAGTGTACGT | 1318 | 3125 | 2525 | 600 |
| 369 | SGSNTGP | 1319 | TCGGGGTCTAATACGGGTCCT | 1320 | 3124 | 2969 | 155 |
| 370 | GTLERTA | 1321 | GGTACTCTTGAGAGGACTGCT | 1322 | 3122 | 2898 | 224 |
| 371 | KLTSEMT | 1323 | AAATTAACATCCGAAATGACC | 1324 | 3121 | 2467 | 654 |
| 372 | DATTKSM | 1325 | GACGCCACAACTAAATCCATG | 1326 | 3120 | 3120 | 0 |
| 373 | GLAGRVV | 1327 | GGGTTGGCGGGGCGTGTTGTT | 1328 | 3117 | 0 | 3117 |
| 374 | AAGGIMN | 1329 | GCTGCCGGGGGGATCATGAAC | 1330 | 3116 | 0 | 3116 |
| 375 | ISDYTTL | 1331 | ATATCAGACTACACAACACTT | 1332 | 3116 | 0 | 3116 |
| 376 | VQHDLTL | 1333 | GTCCAACACGACCTTACCCTT | 1334 | 3116 | 2577 | 540 |
| 377 | MAGSVSK | 1335 | ATGGCTGGTTCGGTATCAAAA | 1336 | 3113 | 1928 | 1184 |
| 378 | SYGSDSK | 1337 | TCTTATGGTTCTGATTCGAAG | 1338 | 3109 | 830 | 2279 |
| 379 | VLSSPGP | 1339 | GTTCTGTCTTCGCCTGGTCCT | 1340 | 3108 | 981 | 2127 |
| 380 | VNSGQQN | 1341 | GTAAACAGCGGCCAACAAAAC | 1342 | 3106 | 2366 | 739 |
| 381 | TSQGAIT | 1343 | ACGTCGCAAGGCGCAATAACC | 1344 | 3106 | 1567 | 1539 |
| 382 | AIGHSQV | 1345 | GCGATTGGTCATAGTCAGGTT | 1346 | 3106 | 3106 | 0 |
| 383 | QADIVGL | 1347 | CAGGCTGATATTGTTGGGCTG | 1348 | 3105 | 1105 | 2000 |
| 384 | SKSNDSS | 1349 | AGTAAGTCTAATGATAGTTCT | 1350 | 3104 | 573 | 2532 |
| 385 | LLAGADR | 1351 | TTGCTTGCTGGTGCTGATCGT | 1352 | 3100 | 2577 | 522 |
| 386 | VYSDRTM | 1353 | GTCTACAGTGACCGCACCATG | 1354 | 3099 | 2930 | 169 |
| 387 | SEGGVKY | 1355 | AGTGAGGGGGGTGTGAAGTAT | 1356 | 3099 | 2806 | 293 |
| 388 | SSDGGKG | 1357 | TCAAGCGACGGCGGCAAAGGA | 1358 | 3096 | 2000 | 1096 |
| 389 | LTHSTAD | 1359 | TTGACCCACTCCACAGCCGAC | 1360 | 3095 | 2372 | 724 |
| 390 | PGGESRG | 1361 | CCTGGTGGGGAGTCTCGGGGG | 1362 | 3094 | 2629 | 465 |
| 391 | PMNGSTR | 1363 | CCGATGAACGGGTCCACTAGG | 1364 | 3089 | 2000 | 1089 |
| 392 | STDGGST | 1365 | TCGACTGATGGTGGTAGTACT | 1366 | 3088 | 2000 | 1088 |
| 393 | EASGMNH | 1367 | GAAGCAAGCGGTATGAACCAC | 1368 | 3086 | 2000 | 1086 |
| 394 | SGDKAAL | 1369 | AGTGGTGATAAGGCTGCGTTG | 1370 | 3086 | 3086 | 0 |
| 395 | MLRGYSQ | 1371 | ATGTTACGCGGGTACTCGCAA | 1372 | 3084 | 233 | 2852 |
| 396 | GGGVEVH | 1373 | GGGGGTGGTGTGGAGGTTCAT | 1374 | 3082 | 914 | 2168 |
| 397 | NVIVNGV | 1375 | AATGTGATTGTGAATGGGGTG | 1376 | 3079 | 1034 | 2044 |
| 398 | WNLDKTH | 1377 | TGGAATCTTGATAAGACTCAT | 1378 | 3075 | 821 | 2254 |
| 399 | AQGGATV | 1379 | GCCCAAGGTGGCGCGACGGTA | 1380 | 3073 | 0 | 3073 |
| 400 | PLINGLV | 1381 | CCACTTATCAACGGCTTAGTT | 1382 | 3073 | 668 | 2405 |
| 401 | RVELTGT | 1383 | CGCGTAGAATTGACCGGCACG | 1384 | 3072 | 2886 | 186 |
| 402 | GEGGTVR | 1385 | GGTGAGGGTGGTACTGTGAGG | 1386 | 3068 | 1614 | 1454 |
| 403 | GAGELSS | 1387 | GGTGCGGGGGAGCTTAGTAGT | 1388 | 3064 | 2000 | 1064 |
| 404 | LGKAVPD | 1389 | CTTGGTAAGGCGGTTCCGGAT | 1390 | 3063 | 2000 | 1063 |
| 405 | WADTKDR | 1391 | TGGGCCGACACGAAAGACCGA | 1392 | 3063 | 2961 | 102 |
| 406 | LAGLGGM | 1393 | CTAGCTGGCCTCGGTGGAATG | 1394 | 3061 | 2769 | 292 |
| 407 | PVLAAGH | 1395 | CCAGTACTAGCGGCTGGGCAC | 1396 | 3056 | 980 | 2076 |
| 408 | SDTIGLR | 1397 | AGTGACACCATAGGCCTCCGC | 1398 | 3056 | 2758 | 298 |
| 409 | GVKETRA | 1399 | GGCGTCAAAGAAACCCGGGCC | 1400 | 3055 | 953 | 2102 |
| 410 | AETMGVA | 1401 | GCAGAAACCATGGGTGTCGCC | 1402 | 3052 | 895 | 2157 |
| 411 | SLTDRAS | 1403 | TCGTTGACGGATAGGGCGTCT | 1404 | 3051 | 1051 | 2000 |
| 412 | RSNGSEN | 1405 | AGGTCGAATGGTTCGGAGAAT | 1406 | 3051 | 2853 | 198 |
| 413 | PGSDGRT | 1407 | CCGGGTTCTGATGGTCGTACT | 1408 | 3051 | 2852 | 199 |
| 414 | TRTEDYT | 1409 | ACTCGCACTGAAGACTACACC | 1410 | 3051 | 3051 | 0 |
| 415 | PSVSVTL | 1411 | CCTTCTGTTTCTGTGACTTTG | 1412 | 3049 | 1047 | 2002 |
| 416 | LSTGAEK | 1413 | CTCTCTACCGGCGCAGAAAAA | 1414 | 3046 | 2000 | 1046 |
| 417 | SETNGVR | 1415 | AGCGAAACGAACGGCGTCCGG | 1416 | 3044 | 408 | 2635 |
| 418 | SPQVYDD | 1417 | AGTCCGCAGGTTTATGATGAT | 1418 | 3042 | 1042 | 2000 |
| 419 | EGGTHVR | 1419 | GAAGGTGGCACCCACGTACGG | 1420 | 3036 | 2865 | 171 |
| 420 | STLGSTR | 1421 | TCGACGTTGGGTAGTACGAGG | 1422 | 3036 | 2829 | 206 |
| 421 | SEGKVGP | 1423 | TCTGAGGGTAAGGTTGGGCCT | 1424 | 3035 | 1318 | 1717 |
| 422 | AVKDELV | 1425 | GCTGTTAAGGATGAGCTGGTG | 1426 | 3032 | 0 | 3032 |
| 423 | TPSGNLM | 1427 | ACGCCGTCGGGGAATCTTATG | 1428 | 3029 | 2000 | 1029 |
| 424 | VMTGTLT | 1429 | GTGATGACGGGGACTTTGACT | 1430 | 3028 | 0 | 3028 |
| 425 | QIASADI | 1431 | CAGATTGCGAGTGCGGATATT | 1432 | 3027 | 0 | 3027 |
| 426 | LYGDGSV | 1433 | CTTTATGGTGATGGTAGTGTT | 1434 | 3024 | 2000 | 1024 |
| 427 | PTQLQRV | 1435 | CCGACTCAGCTGCAGCGTGTG | 1436 | 3023 | 0 | 3023 |
| 428 | SNVQDGL | 1437 | AGCAACGTCCAAGACGGACTA | 1438 | 3014 | 0 | 3014 |
| 429 | YDAKVGH | 1439 | TACGACGCAAAAGTGGGGCAC | 1440 | 3014 | 2942 | 71 |
| 430 | AVGLDNR | 1441 | GCTGTGGGTCTGGATAATCGT | 1442 | 3012 | 908 | 2104 |
| 431 | NSNDVHM | 1443 | AACTCTAACGACGTCCACATG | 1444 | 3011 | 0 | 3011 |
| 432 | PSGALMT | 1445 | CCGAGTGGTGCGCTGATGACT | 1446 | 3010 | 1010 | 2000 |
| 433 | SQEQVSA | 1447 | AGTCAGGAGCAGGTGAGTGCT | 1448 | 3009 | 1967 | 1042 |
| 434 | GSGSDGV | 1449 | GGGAGTGGTAGTGATGGGGTT | 1450 | 3008 | 1008 | 2000 |
| 435 | VDTSDRV | 1451 | GTTGATACTAGTGATCGTGTT | 1452 | 3007 | 3006 | 1 |
| 436 | ATMSPTT | 1453 | GCCACAATGTCCCCTACAACG | 1454 | 3006 | 3006 | 0 |
| 437 | TPTLPFI | 1455 | ACACCCACCCTGCCATTCATA | 1456 | 2999 | 0 | 2999 |
| 438 | AVKEYDS | 1457 | GCGGTGAAGGAGTATGATTCG | 1458 | 2999 | 2779 | 220 |
| 439 | LSLPDGD | 1459 | CTCTCCTTACCAGACGGAGAC | 1460 | 2998 | 918 | 2080 |
| 440 | AEKSGMV | 1461 | GCCGAAAAATCTGGTATGGTC | 1462 | 2997 | 2748 | 249 |
| 441 | LNVDTGS | 1463 | CTCAACGTGGACACTGGTTCA | 1464 | 2989 | 2634 | 355 |
| 442 | PTQGTPR | 1465 | CCAACGCAAGGAACACCGCGA | 1466 | 2988 | 2988 | 0 |
| 443 | PVGTNAR | 1467 | CCTGTCGGCACAAACGCAAGA | 1468 | 2987 | 899 | 2088 |
| 444 | NGGIITR | 1469 | AACGGAGGTATCATCACCCGC | 1470 | 2983 | 1208 | 1775 |
| 445 | VVGTQDR | 1471 | GTTGTGGGGACTCAGGATAGG | 1472 | 2980 | 0 | 2980 |
| 446 | NTLHTST | 1473 | AATACTCTTCATACTTCGACT | 1474 | 2980 | 2637 | 342 |
| 447 | VGSLGTD | 1475 | GTAGGCTCGCTGGGTACTGAC | 1476 | 2973 | 1388 | 1585 |
| 448 | LSTVTGQ | 1477 | CTTTCGACGGTGACTGGGCAG | 1478 | 2970 | 980 | 1989 |
| 449 | VLTNTTT | 1479 | GTGCTTACGAATACTACTACT | 1480 | 2964 | 2680 | 283 |
| 450 | SGDKAAL | 1481 | TCCGGCGACAAAGCCGCACTT | 1482 | 2959 | 2518 | 441 |
| 451 | YQTETNN | 1483 | TATCAGACTGAGACGAATAAT | 1484 | 2957 | 2263 | 694 |
| 452 | SVGLVAG | 1485 | AGTGTGGGTTTGGTGGCTGGT | 1486 | 2951 | 784 | 2167 |
| 453 | RMTGDLT | 1487 | CGTATGACTGGAGACCTAACC | 1488 | 2951 | 2000 | 951 |
| 454 | TSGNLTW | 1489 | ACTTCTGGTAATTTGACGTGG | 1490 | 2950 | 0 | 2950 |
| 455 | ANVNVKV | 1491 | GCGAATGTTAATGTGAAGGTG | 1492 | 2948 | 1494 | 1454 |
| 456 | VNVTMTT | 1493 | GTTAATGTGACGATGACTACG | 1494 | 2947 | 1764 | 1182 |
| 457 | PGVSVTS | 1495 | CCTGGTGTGAGTGTGACTTCT | 1496 | 2946 | 0 | 2946 |
| 458 | TGDRDQF | 1497 | ACCGGCGACAGAGACCAATTC | 1498 | 2942 | 2383 | 560 |
| 459 | SKAEGPV | 1499 | AGTAAAGCCGAAGGACCTGTC | 1500 | 2942 | 2787 | 154 |
| 460 | DSAPAAR | 1501 | GATTCGGCTCCGGCGGCTCGG | 1502 | 2941 | 2498 | 443 |
| 461 | SRDDGRM | 1503 | TCGCGTGATGATGGGAGGATG | 1504 | 2939 | 2687 | 252 |
| 462 | IPEGSVR | 1505 | ATCCCTGAAGGATCAGTACGA | 1506 | 2938 | 938 | 2000 |
| 463 | VTTVSLV | 1507 | GTAACGACTGTGTCCCTAGTT | 1508 | 2933 | 2817 | 116 |
| 464 | PIHGASS | 1509 | CCGATTCATGGTGCTAGTTCG | 1510 | 2932 | 2343 | 589 |
| 465 | VSASISK | 1511 | GTTTCGGCGAGTATTTCTAAG | 1512 | 2931 | 919 | 2012 |
| 466 | TKDNGVM | 1513 | ACTAAGGATAATGGTGTGATG | 1514 | 2930 | 0 | 2930 |
| 467 | LGASVPK | 1515 | CTAGGCGCCTCCGTCCCCAAA | 1516 | 2929 | 775 | 2154 |
| 468 | IEGLGGL | 1517 | ATAGAAGGCCTCGGAGGTTTG | 1518 | 2929 | 2705 | 224 |
| 469 | VSGGDYS | 1519 | GTGAGTGGGGGTGATTATTCG | 1520 | 2927 | 1 | 2926 |
| 470 | SEKQKVR | 1521 | AGTGAGAAGCAGAAGGTTAGG | 1522 | 2927 | 0 | 2927 |
| 471 | PGRVSSE | 1523 | CCTGGTCGGGTCAGCTCAGAA | 1524 | 2927 | 927 | 2000 |
| 472 | DRITLGT | 1525 | GATCGGATTACGTTGGGGACG | 1526 | 2923 | 0 | 2923 |
| 473 | ANNGTTW | 1527 | GCTAATAATGGGACGACTTGG | 1528 | 2923 | 2636 | 288 |
| 474 | TSVISQV | 1529 | ACGAGTGTTATTTCGCAGGTT | 1530 | 2922 | 2922 | 0 |
| 475 | LANMMSV | 1531 | CTGGCTAATATGATGAGTGTT | 1532 | 2919 | 2919 | 0 |
| 476 | EIVLTVP | 1533 | GAAATAGTGCTGACCGTCCCC | 1534 | 2917 | 0 | 2917 |
| 477 | VHKDQEI | 1535 | GTGCATAAGGATCAGGAGATT | 1536 | 2917 | 2385 | 532 |
| 478 | QVGDSTL | 1537 | CAAGTAGGAGACTCGACGTTA | 1538 | 2917 | 2917 | 0 |
| 479 | SEKSVPL | 1539 | AGTGAGAAGAGTGTGCCGCTT | 1540 | 2903 | 2000 | 903 |
| 480 | SNHDLTH | 1541 | TCCAACCACGACCTTACCCAC | 1542 | 2902 | 2902 | 0 |
| 481 | NQLAEMV | 1543 | AACCAACTGGCTGAAATGGTG | 1544 | 2899 | 661 | 2238 |
| 482 | QMTHGLI | 1545 | CAGATGACTCATGGGCTTATT | 1546 | 2897 | 0 | 2897 |
| 483 | QVSDNKT | 1547 | CAAGTATCCGACAACAAAACC | 1548 | 2897 | 262 | 2635 |
| 484 | GGTNSAH | 1549 | GGGGGTACGAATAGTGCTCAT | 1550 | 2897 | 311 | 2587 |
| 485 | GAADRMQ | 1551 | GGGGCGGCGGATCGGATGCAG | 1552 | 2897 | 711 | 2187 |
| 486 | ESGVHQK | 1553 | GAGTCTGGTGTTCATCAGAAG | 1554 | 2896 | 511 | 2385 |
| 487 | GGTGALR | 1555 | GGCGGCACAGGGGCTCTCAGA | 1556 | 2896 | 1776 | 1119 |
| 488 | NTLGVAY | 1557 | AACACATTAGGCGTGGCATAC | 1558 | 2895 | 2000 | 895 |
| 489 | VQHSQDN | 1559 | GTGCAGCATTCGCAGGATAAT | 1560 | 2894 | 2775 | 119 |
| 490 | EYNTRDK | 1561 | GAGTATAATACTCGGGATAAG | 1562 | 2894 | 894 | 2000 |
| 491 | RSEVNGV | 1563 | CGGAGTGAGGTGAATGGGGTT | 1564 | 2889 | 1768 | 1122 |
| 492 | GLAETRA | 1565 | GGGCTTGCTGAGACTAGGGCT | 1566 | 2885 | 2135 | 750 |
| 493 | MNGGYVL | 1567 | ATGAACGGCGGATACGTACTT | 1568 | 2882 | 811 | 2071 |
| 494 | TSGNAGL | 1569 | ACTTCTGGTAATGCTGGGCTT | 1570 | 2881 | 2315 | 566 |
| 495 | NTTQTSW | 1571 | AACACTACACAAACGTCCTGG | 1572 | 2880 | 725 | 2156 |
| 496 | SLAGGTP | 1573 | TCGTTGGCTGGTGGTACTCCT | 1574 | 2879 | 595 | 2284 |
| 497 | MTGHDAV | 1575 | ATGACTGGGCATGATGCTGTG | 1576 | 2879 | 2746 | 133 |
| 498 | DETRTHI | 1577 | GACGAAACCCGGACACACATA | 1578 | 2878 | 2878 | 0 |
| 499 | DMLNNTA | 1579 | GACATGTTAAACAACACTGCT | 1580 | 2872 | 0 | 2872 |
| 500 | THNENMF | 1581 | ACTCACAACGAAAACATGTTC | 1582 | 2872 | 0 | 2872 |
| 501 | RNLDLTH | 1583 | CGTAACTTGGACTTAACGCAC | 1584 | 2871 | 0 | 2871 |
| 502 | RDNVEST | 1585 | AGGGACAACGTGGAATCAACA | 1586 | 2868 | 1357 | 1511 |
| 503 | NVVSLAT | 1587 | AATGTTGTGAGTCTGGCTACT | 1588 | 2864 | 1707 | 1157 |
| 504 | REMGQNA | 1589 | CGTGAGATGGGGCAGAATGCT | 1590 | 2858 | 2349 | 509 |
| 505 | AVVSAGP | 1591 | GCGGTGGTGTCTGCTGGGCCG | 1592 | 2855 | 0 | 2855 |
| 506 | LTGISLV | 1593 | CTTACAGGAATATCTCTCGTT | 1594 | 2853 | 1470 | 1383 |
| 507 | VLTVGSV | 1595 | GTACTCACGGTCGGGAGCGTG | 1596 | 2853 | 2682 | 171 |
| 508 | HRDSAEP | 1597 | CACAGGGACTCGGCGGAACCC | 1598 | 2851 | 2000 | 851 |
| 509 | VAALGMS | 1599 | GTTGCTGCTTTGGGTATGTCT | 1600 | 2850 | 0 | 2850 |
| 510 | TTSENLM | 1601 | ACGACGTCGGAGAATCTTATG | 1602 | 2848 | 2848 | 0 |
| 511 | YTAGSQA | 1603 | TACACTGCTGGCAGTCAAGCC | 1604 | 2845 | 1405 | 1440 |
| 512 | LSTLLGA | 1605 | TTGAGTACGCTACTCGGAGCC | 1606 | 2843 | 0 | 2843 |
| 513 | MSISEPR | 1607 | ATGAGTATCTCTGAACCCCGT | 1608 | 2840 | 1110 | 1730 |
| 514 | VMDHKST | 1609 | GTCATGGACCACAAATCTACA | 1610 | 2840 | 2840 | 0 |
| 515 | IKSDERL | 1611 | ATAAAAAGCGACGAACGTTTA | 1612 | 2840 | 2840 | 0 |
| 516 | TTEKHTG | 1613 | ACCACGGAAAAACACACGGGC | 1614 | 2839 | 0 | 2839 |
| 517 | ASPQGVR | 1615 | GCTAGTCCGCAGGGGGTTCGT | 1616 | 2838 | 2000 | 839 |
| 518 | PGQHNQA | 1617 | CCGGGTCAGCATAATCAGGCT | 1618 | 2837 | 881 | 1956 |
| 519 | PNLGNPS | 1619 | CCGAATCTGGGTAATCCTAGT | 1620 | 2836 | 2000 | 836 |
| 520 | RLTEADR | 1621 | CGGTTGACTGAGGCGGATCGT | 1622 | 2835 | 2699 | 136 |
| 521 | WNHSSTV | 1623 | TGGAACCACTCATCAACCGTG | 1624 | 2832 | 601 | 2230 |
| 522 | IGNALLK | 1625 | ATTGGTAATGCGTTGCTGAAG | 1626 | 2831 | 2410 | 420 |
| 523 | TAADHLR | 1627 | ACTGCTGCTGATCATTTGAGG | 1628 | 2830 | 718 | 2112 |
| 524 | MVSNDTS | 1629 | ATGGTTAGCAACGACACTAGC | 1630 | 2828 | 0 | 2828 |
| 525 | IQDSVQF | 1631 | ATTCAGGATTCGGTTCAGTTT | 1632 | 2827 | 370 | 2456 |
| 526 | MGENLPS | 1633 | ATGGGGGAGAATCTTCCGAGT | 1634 | 2826 | 293 | 2533 |
| 527 | ADSTQGK | 1635 | GCTGATTCGACTCAGGGTAAG | 1636 | 2826 | 1718 | 1108 |
| 528 | IPVDMNR | 1637 | ATCCCCGTCGACATGAACAGG | 1638 | 2825 | 467 | 2358 |
| 529 | SVDSGRL | 1639 | AGTGTTGATAGTGGGCGGCTT | 1640 | 2825 | 665 | 2160 |
| 530 | SYGDGGV | 1641 | TCTTACGGGGACGGAGGGGTC | 1642 | 2825 | 2521 | 304 |
| 531 | SHSLIEV | 1643 | TCTCATAGTCTGATTGAGGTG | 1644 | 2823 | 744 | 2079 |
| 532 | LAGLGVI | 1645 | CTGGCGGGGCTGGGCGTCATA | 1646 | 2823 | 623 | 2200 |
| 533 | MSVGQSW | 1647 | ATGTCAGTGGGTCAATCTTGG | 1648 | 2822 | 676 | 2146 |
| 534 | TRDGQLA | 1649 | ACCAGGGACGGACAACTCGCA | 1650 | 2820 | 0 | 2820 |
| 535 | AQEVARA | 1651 | GCGCAGGAGGTGGCGCGGGCT | 1652 | 2820 | 447 | 2374 |
| 536 | GSVGVVV | 1653 | GGTTCGGTGGGTGTGGTTGTG | 1654 | 2818 | 0 | 2818 |
| 537 | GGTLLTV | 1655 | GGTGGGACGTTGTTGACGGTG | 1656 | 2818 | 1284 | 1534 |
| 538 | ITENVSR | 1657 | ATCACGGAAAACGTAAGCCGT | 1658 | 2817 | 2270 | 546 |
| 539 | SAGVIMN | 1659 | TCGGCGGGTGTTATTATGAAT | 1660 | 2817 | 2287 | 529 |
| 540 | AEGLRGQ | 1661 | GCTGAGGGGCTTCGGGGGCAG | 1662 | 2814 | 2814 | 0 |
| 541 | GTGEIGM | 1663 | GGGACTGGTGAGATTGGTATG | 1664 | 2813 | 2813 | 0 |
| 542 | VLGAHET | 1665 | GTACTAGGAGCGCACGAAACA | 1666 | 2812 | 2000 | 812 |
| 543 | MALGYSS | 1667 | ATGGCGTTGGGGTATAGTAGT | 1668 | 2811 | 0 | 2811 |
| 544 | STVTGGP | 1669 | AGCACCGTCACGGGCGGACCC | 1670 | 2811 | 2811 | 0 |
| 545 | MQGENNK | 1671 | ATGCAGGGTGAGAATAATAAG | 1672 | 2810 | 936 | 1874 |
| 546 | AAAQTAT | 1673 | GCAGCAGCCCAAACCGCAACG | 1674 | 2810 | 1000 | 1810 |
| 547 | VNSGSVL | 1675 | GTGAATTCGGGTTCTGTGCTG | 1676 | 2808 | 2536 | 272 |
| 548 | HAAGDRS | 1677 | CATGCGGCGGGTGATCGTAGT | 1678 | 2806 | 1299 | 1508 |
| 549 | PVESQTA | 1679 | CCCGTTGAAAGCCAAACTGCC | 1680 | 2805 | 480 | 2325 |
| 550 | STIPSLM | 1681 | AGCACAATACCCTCATTAATG | 1682 | 2801 | 0 | 2801 |
| 551 | KNAGAES | 1683 | AAGAATGCGGGTGCTGAGAGT | 1684 | 2800 | 650 | 2150 |
| 552 | SPGSDSK | 1685 | TCTCCTGGTTCTGATTCGAAG | 1686 | 2795 | 679 | 2116 |
| 553 | KVTLEGD | 1687 | AAAGTCACACTAGAAGGTGAC | 1688 | 2793 | 2375 | 418 |
| 554 | PQGNSSV | 1689 | CCCCAAGGCAACAGCTCAGTC | 1690 | 2791 | 2565 | 226 |
| 555 | VLGTTTP | 1691 | GTGCTTGGGACGACTACGCCT | 1692 | 2791 | 2464 | 327 |
| 556 | LSNHGPI | 1693 | TTGTCTAACCACGGCCCCATA | 1694 | 2791 | 2791 | 0 |
| 557 | KENRVSD | 1695 | AAAGAAAACAGGGTAAGTGAC | 1696 | 2791 | 2791 | 0 |
| 558 | IGGNSGD | 1697 | ATTGGGGGGAATAGTGGGGAT | 1698 | 2788 | 1118 | 1670 |
| 559 | GAIGPAT | 1699 | GGGGCTATTGGTCCTGCTACT | 1700 | 2786 | 0 | 2786 |
| 560 | DTHAKSM | 1701 | GATACGCATGCTAAGAGTATG | 1702 | 2784 | 2784 | 0 |
| 561 | MNGGHHL | 1703 | ATGAATGGGGGTCATCATCTG | 1704 | 2783 | 718 | 2065 |
| 562 | AAGLIQN | 1705 | GCTGCCGGGCTGATACAAAAC | 1706 | 2780 | 2393 | 387 |
| 563 | QARDTKT | 1707 | CAAGCTCGAGACACCAAAACA | 1708 | 2780 | 2780 | 0 |
| 564 | SSYANEH | 1709 | AGTTCGTATGCTAATGAGCAT | 1710 | 2779 | 2659 | 120 |
| 565 | VLVSDRA | 1711 | GTGTTGGTTTCGGATCGTGCT | 1712 | 2778 | 778 | 2000 |
| 566 | WSTDGGS | 1713 | TGGTCAACGGACGGCGGGAGT | 1714 | 2773 | 0 | 2773 |
| 567 | CRESTCV | 1715 | TGTCGTGAGTCGACGTGTGTT | 1716 | 2772 | 2581 | 191 |
| 568 | SQAEGPV | 1717 | AGTCAAGCGGAAGGGCCCGTG | 1718 | 2764 | 0 | 2764 |
| 569 | ALSNDKH | 1719 | GCGCTTAGTAACGACAAACAC | 1720 | 2764 | 2208 | 556 |
| 570 | LGASVPM | 1721 | CTTGGTGCTTCGGTTCCGATG | 1722 | 2763 | 2242 | 521 |
| 571 | YSVGDSI | 1723 | TATTCTGTTGGGGATAGTATT | 1724 | 2762 | 2000 | 762 |
| 572 | QNSTGLW | 1725 | CAAAACTCCACAGGCCTCTGG | 1726 | 2757 | 2432 | 325 |
| 573 | LVAGQDL | 1727 | CTGGTGGCGGGGCAGGATTTG | 1728 | 2757 | 2757 | 0 |
| 574 | GLSEGSV | 1729 | GGCTTAAGCGAAGGCTCAGTA | 1730 | 2756 | 1401 | 1355 |
| 575 | TLAISEM | 1731 | ACTTTGGCGATTTCTGAGATG | 1732 | 2755 | 441 | 2314 |
| 576 | LVHTTNN | 1733 | CTAGTACACACAACCAACAAC | 1734 | 2753 | 2753 | 0 |
| 577 | TVVSSTR | 1735 | ACTGTTGTGTCTTCTACGAGG | 1736 | 2750 | 473 | 2277 |
| 578 | GRGPDLT | 1737 | GGCAGGGGACCAGACCTCACT | 1738 | 2748 | 631 | 2117 |
| 579 | IQSDHGR | 1739 | ATCCAAAGTGACCACGGACGC | 1740 | 2748 | 2748 | 0 |
| 580 | QSSEMRD | 1741 | CAATCGTCCGAAATGCGTGAC | 1742 | 2743 | 596 | 2147 |
| 581 | GSSENVS | 1743 | GGAAGCTCCGAAAACGTATCT | 1744 | 2740 | 2000 | 740 |
| 582 | KTPGVDP | 1745 | AAGACTCCGGGGGTGGATCCT | 1746 | 2740 | 2368 | 372 |
| 583 | QADDHGR | 1747 | CAGGCGGATGATCATGGTAGG | 1748 | 2739 | 709 | 2030 |
| 584 | AYSDGSS | 1749 | GCTTATAGTGATGGGTCGTCT | 1750 | 2738 | 1944 | 794 |
| 585 | MAASMTN | 1751 | ATGGCTGCCTCGATGACAAAC | 1752 | 2737 | 0 | 2737 |
| 586 | STIPTLT | 1753 | AGTACTATTCCTACTCTGACG | 1754 | 2733 | 0 | 2733 |
| 587 | AEVLNAL | 1755 | GCGGAGGTGCTGAATGCTTTG | 1756 | 2733 | 2444 | 289 |
| 588 | AESLSGL | 1757 | GCTGAGAGTTTGAGTGGGTTG | 1758 | 2730 | 1791 | 939 |
| 589 | NQGGGLT | 1759 | AACCAAGGTGGCGGCTTAACA | 1760 | 2730 | 2730 | 0 |
| 590 | AHNGGVQ | 1761 | GCTCATAATGGTGGTGTTCAG | 1762 | 2727 | 2587 | 140 |
| 591 | MGGSVTI | 1763 | ATGGGGGGTAGTGTTACGATT | 1764 | 2726 | 2726 | 0 |
| 592 | VSVSMGI | 1765 | GTTAGTGTGAGTATGGGCATC | 1766 | 2724 | 578 | 2146 |
| 593 | SGVAYER | 1767 | AGTGGGGTGGCTTATGAGAGG | 1768 | 2722 | 2274 | 448 |
| 594 | PSATQSL | 1769 | CCGTCTGCGACTCAGTCGTTG | 1770 | 2722 | 537 | 2185 |
| 595 | STGENKD | 1771 | TCCACGGGCGAAAACAAAGAC | 1772 | 2721 | 2657 | 64 |
| 596 | PTQGTLG | 1773 | CCTACTCAGGGGACGCTTGGG | 1774 | 2720 | 720 | 2000 |
| 597 | VLSADSV | 1775 | GTGCTTTCGGCTGATTCTGTG | 1776 | 2720 | 2720 | 0 |
| 598 | LGETLIR | 1777 | TTGGGGGAGACTCTGATTCGG | 1778 | 2718 | 2718 | 0 |
| 599 | EHLAGVV | 1779 | GAGCATTTGGCTGGTGTTGTT | 1780 | 2717 | 2600 | 116 |
| 600 | QSDNHGR | 1781 | CAAAGCGACAACCACGGGCGG | 1782 | 2716 | 2716 | 0 |
| 601 | LSVSQSA | 1783 | CTGAGTGTTTCTCAGTCGGCG | 1784 | 2714 | 2384 | 330 |
| 602 | SELSLGY | 1785 | AGCGAATTGAGTCTCGGCTAC | 1786 | 2713 | 2713 | 0 |
| 603 | AEDKANS | 1787 | GCTGAGGATAAGGCGAATAGT | 1788 | 2712 | 360 | 2352 |
| 604 | STINTLM | 1789 | TCGACAATAAACACCCTAATG | 1790 | 2711 | 1556 | 1155 |
| 605 | TGMTLGT | 1791 | ACGGGGATGACGCTGGGTACG | 1792 | 2708 | 1246 | 1462 |
| 606 | LNGGHVL | 1793 | TTGAATGGGGGTCATGTTCTG | 1794 | 2702 | 2460 | 242 |
| 607 | VVSDAGK | 1795 | GTGGTGAGTGATGCTGGGAAG | 1796 | 2700 | 317 | 2383 |
| 608 | MNPSNSM | 1797 | ATGAATCCTAGTAATTCGATG | 1798 | 2697 | 2572 | 125 |
| 609 | TAASIQS | 1799 | ACGGCCGCAAGCATACAATCC | 1800 | 2696 | 0 | 2696 |
| 610 | RGTEHLM | 1801 | CGTGGTACTGAGCATTTGATG | 1802 | 2695 | 219 | 2476 |
| 611 | LLADKSV | 1803 | CTTCTTGCTGATAAGAGTGTG | 1804 | 2695 | 473 | 2222 |
| 612 | PGEHNHA | 1805 | CCGGGTGAGCATAATCATGCT | 1806 | 2693 | 2436 | 256 |
| 613 | GTTSDTY | 1807 | GGTACTACGTCGGATACTTAT | 1808 | 2691 | 2400 | 291 |
| 614 | GDISARY | 1809 | GGTGATATTTCTGCGAGGTAT | 1810 | 2688 | 2000 | 688 |
| 615 | FSVSSLS | 1811 | TTCTCCGTCTCAAGTTTATCC | 1812 | 2688 | 688 | 2000 |
| 616 | TSDRDQY | 1813 | ACTAGTGATCGGGATCAGTAT | 1814 | 2688 | 2339 | 349 |
| 617 | AHVHVKE | 1815 | GCGCATGTTCATGTGAAGGAG | 1816 | 2688 | 2688 | 0 |
| 618 | GRDLSTA | 1817 | GGTCGGGATCTTTCGACTGCT | 1818 | 2687 | 680 | 2007 |
| 619 | GGGTEFY | 1819 | GGAGGAGGCACTGAATTCTAC | 1820 | 2683 | 2683 | 0 |
| 620 | PYPSNSH | 1821 | CCGTATCCGAGTAATTCGCAT | 1822 | 2682 | 2682 | 0 |
| 621 | NLGVGQM | 1823 | AATTTGGGTGTGGGTCAGATG | 1824 | 2680 | 2529 | 151 |
| 622 | LSPGTDK | 1825 | CTGTCGCCGGGGACGGATAAG | 1826 | 2680 | 2362 | 318 |
| 623 | GTDRVSR | 1827 | GGCACAGACAGAGTATCCCGT | 1828 | 2679 | 2679 | 0 |
| 624 | MADGASM | 1829 | ATGGCGGATGGTGCGTCTATG | 1830 | 2678 | 587 | 2091 |
| 625 | AGISNQT | 1831 | GCCGGAATCTCTAACCAAACT | 1832 | 2676 | 2520 | 156 |
| 626 | ETQGRQF | 1833 | GAGACTCAGGGTCGTCAGTTT | 1834 | 2675 | 284 | 2391 |
| 627 | YGSNDLS | 1835 | TATGGGAGTAATGATCTGAGT | 1836 | 2674 | 2262 | 412 |
| 628 | AADNNRW | 1837 | GCTGCTGATAATAATAGGTGG | 1838 | 2674 | 326 | 2348 |
| 629 | MPSNGQV | 1839 | ATGCCGTCTAATGGGCAGGTT | 1840 | 2674 | 674 | 2000 |
| 630 | GFGDGTR | 1841 | GGCTTCGGAGACGGTACACGC | 1842 | 2674 | 2674 | 0 |
| 631 | RLNEHEA | 1843 | AGGTTAAACGAACACGAAGCC | 1844 | 2671 | 2620 | 51 |
| 632 | SVKSVTL | 1845 | AGTGTGAAGAGTGTGACGCTT | 1846 | 2667 | 2000 | 667 |
| 633 | LTDGYTP | 1847 | CTGACCGACGGTTACACACCG | 1848 | 2667 | 2357 | 310 |
| 634 | SNIGNDR | 1849 | TCGAATATTGGGAATGATAGG | 1850 | 2666 | 362 | 2304 |
| 635 | KESTLST | 1851 | AAAGAAAGTACCCTCTCAACA | 1852 | 2666 | 896 | 1770 |
| 636 | RVDPAQL | 1853 | AGGGTGGATCCGGCGCAGCTT | 1854 | 2666 | 2000 | 666 |
| 637 | MYGESAK | 1855 | ATGTACGGGGAAAGCGCTAAA | 1856 | 2665 | 2665 | 0 |
| 638 | VAEGGQI | 1857 | GTGGCGGAGGGTGGGCAGATT | 1858 | 2664 | 489 | 2175 |
| 639 | LTDRVSR | 1859 | CTAACCGACAGAGTCTCTCGA | 1860 | 2664 | 0 | 2664 |
| 640 | RLDELMI | 1861 | CGATTGGACGAACTAATGATC | 1862 | 2663 | 0 | 2663 |
| 641 | PVKEYES | 1863 | CCGGTGAAGGAGTATGAGTCG | 1864 | 2661 | 1040 | 1621 |
| 642 | QGGDSGG | 1865 | CAAGGGGGAGACTCAGGTGGC | 1866 | 2657 | 2543 | 114 |
| 643 | VHTEAPY | 1867 | GTTCATACGGAGGCTCCGTAT | 1868 | 2651 | 0 | 2651 |
| 644 | SQELRDR | 1869 | AGTCAGGAGCTGAGGGATCGT | 1870 | 2650 | 2516 | 134 |
| 645 | VSRENVS | 1871 | GTCTCGCGTGAAAACGTTTCC | 1872 | 2648 | 2648 | 0 |
| 646 | STDLSEL | 1873 | TCTACGGATTTGTCGGAGTTG | 1874 | 2645 | 2501 | 144 |
| 647 | GTGIQTR | 1875 | GGCACAGGAATCCAAACACGT | 1876 | 2643 | 2000 | 643 |
| 648 | ADSDYTE | 1877 | GCAGACTCCGACTACACAGAA | 1878 | 2642 | 2000 | 642 |
| 649 | VDTSARD | 1879 | GTCGACACGTCTGCAAGAGAC | 1880 | 2641 | 0 | 2641 |
| 650 | LGNKDGV | 1881 | TTGGGGAATAAGGATGGTGTT | 1882 | 2641 | 641 | 2000 |
| 651 | REAGTNS | 1883 | CGGGAGGCTGGGACGAATTCT | 1884 | 2640 | 2640 | 0 |
| 652 | PGATNNP | 1885 | CCGGGTGCGACGAATAATCCG | 1886 | 2636 | 1686 | 950 |
| 653 | NSISLIN | 1887 | AACTCTATCAGCCTCATAAAC | 1888 | 2636 | 2000 | 636 |
| 654 | ASSEFKI | 1889 | GCCTCATCCGAATTCAAAATA | 1890 | 2635 | 2328 | 307 |
| 655 | VTTGSPV | 1891 | GTTACGACTGGGTCGCCGGTA | 1892 | 2633 | 2078 | 555 |
| 656 | GSTNVNV | 1893 | GGTAGTACGAATGTTAATGTG | 1894 | 2631 | 0 | 2631 |
| 657 | GDMSGSL | 1895 | GGGGATATGAGTGGGAGTTTG | 1896 | 2631 | 2631 | 0 |
| 658 | SRTDSGP | 1897 | AGTCGTACGGATTCGGGGCCG | 1898 | 2630 | 2000 | 630 |
| 659 | EKGSTLV | 1899 | GAGAAGGGGTCGACGTTGGTG | 1900 | 2629 | 629 | 2000 |
| 660 | GHATDSV | 1901 | GGTCATGCTACTGATAGTGTG | 1902 | 2628 | 628 | 2000 |
| 661 | VSNGTFV | 1903 | GTGTCGAACGGAACGTTCGTA | 1904 | 2626 | 2000 | 626 |
| 662 | SAGGSLQ | 1905 | TCGGCAGGAGGTAGCCTACAA | 1906 | 2626 | 0 | 2626 |
| 663 | HDTSDSV | 1907 | CATGATACTAGTGATAGTGTT | 1908 | 2626 | 467 | 2160 |
| 664 | AAGVSLN | 1909 | GCGGCGGGTGTTAGTCTGAAT | 1910 | 2626 | 2626 | 0 |
| 665 | NTVTNIL | 1911 | AACACCGTCACGAACATCCTC | 1912 | 2624 | C | 2624 |
| 666 | KSHSENN | 1913 | AAATCACACTCAGAAAACAAC | 1914 | 2624 | 1458 | 1166 |
| 667 | RNHDLTH | 1915 | AGGAATCATGATCTGACTCAT | 1916 | 2624 | 2624 | 0 |
| 668 | VKDGPGT | 1917 | GTGAAAGACGGACCCGGTACG | 1918 | 2623 | 2340 | 283 |
| 669 | VVVGNVK | 1919 | GTTGTTGTTGGTAATGTGAAG | 1920 | 2623 | 2623 | 0 |
| 670 | AGGGDTR | 1921 | GCTGGTGGGGGCGACACACGT | 1922 | 2623 | 2623 | 0 |
| 671 | KSISGEW | 1923 | AAGAGTATTTCGGGTGAGTGG | 1924 | 2622 | 622 | 2000 |
| 672 | ASADSRS | 1925 | GCTTCGGCGGATTCTCGTAGT | 1926 | 2620 | 436 | 2184 |
| 673 | SVAQNQT | 1927 | TCCGTAGCTCAAAACCAAACT | 1928 | 2620 | 620 | 2000 |
| 674 | DRASSDA | 1929 | GACAGGGCTTCATCAGACGCC | 1930 | 2620 | 2481 | 139 |
| 675 | PVRDTKT | 1931 | CCGGTGCGTGATACTAAGACT | 1932 | 2620 | 1696 | 924 |
| 676 | GVGNTNI | 1933 | GGTGTGGGGAATACTAATATT | 1934 | 2619 | 2466 | 154 |
| 677 | GARLTYT | 1935 | GGAGCCCGCCTCACTTACACA | 1936 | 2619 | 750 | 1869 |
| 678 | TSLGLMV | 1937 | ACGAGTCTGGGTCTTATGGTG | 1938 | 2618 | 0 | 2618 |
| 679 | KAVDNGL | 1939 | AAGGCTGTTGATAATGGGCTG | 1940 | 2616 | 616 | 2000 |
| 680 | AHEAGSR | 1941 | GCTCATGAGGCGGGTAGTCGT | 1942 | 2615 | 2525 | 90 |
| 681 | ASQDRGL | 1943 | GCTTCGCAGGATAGGGGGTTG | 1944 | 2611 | 128 | 2483 |
| 682 | SVTDIKH | 1945 | TCTGTTACTGATATTAAGCAT | 1946 | 2609 | 2000 | 609 |
| 683 | ISNGTER | 1947 | ATATCAAACGGAACAGAACGC | 1948 | 2608 | 0 | 2608 |
| 684 | GHQNGGI | 1949 | GGGCACCAAAACGGCGGAATC | 1950 | 2608 | 779 | 1828 |
| 685 | GNGTGVI | 1951 | GGTAATGGGACTGGTGTGATT | 1952 | 2607 | 2524 | 83 |
| 686 | AKTNDSN | 1953 | GCGAAGACGAATGATAGTAAT | 1954 | 2605 | 2028 | 577 |
| 687 | GMATQTT | 1955 | GGGATGGCTACTCAGACGACT | 1956 | 2602 | 0 | 2602 |
| 688 | SSDTTLR | 1957 | AGTTCGGATACTACTTTGCGT | 1958 | 2602 | 2392 | 209 |
| 689 | LVDDKAH | 1959 | TTGGTTGATGATAAGGCGCAT | 1960 | 2602 | 2003 | 599 |
| 690 | TLAISQP | 1961 | ACCTTAGCCATATCGCAACCT | 1962 | 2600 | 2430 | 170 |
| 691 | AGFSSQS | 1963 | GCGGGGTTTAGTTCTCAGTCG | 1964 | 2597 | 2000 | 597 |
| 692 | ILIGTSP | 1965 | ATTCTTATTGGTACTTCGCCG | 1966 | 2597 | 356 | 2242 |
| 693 | SMESSSR | 1967 | TCTATGGAAAGCAGTTCGCGT | 1968 | 2596 | 2278 | 318 |
| 694 | ARSEFKT | 1969 | GCGAGGTCTGAGTTTAAGACT | 1970 | 2595 | 2595 | 0 |
| 695 | NKSDHEF | 1971 | AACAAATCAGACCACGAATTC | 1972 | 2594 | 2466 | 128 |
| 696 | VEVPTAN | 1973 | GTTGAAGTGCCAACAGCGAAC | 1974 | 2593 | 369 | 2224 |
| 697 | LLTSAVA | 1975 | CTGCTTACATCGGCTGTTGCC | 1976 | 2592 | 2355 | 237 |
| 698 | MGGVSNP | 1977 | ATGGGGGGGGTTAGTAATCCG | 1978 | 2592 | 592 | 2000 |
| 699 | LGDSASP | 1979 | CTTGGGGATTCTGCTTCGCCG | 1980 | 2587 | 2000 | 587 |
| 700 | MVGGGVS | 1981 | ATGGTGGGTGGTGGGGTGTCG | 1982 | 2587 | 587 | 2000 |
| 701 | ASQLTQT | 1983 | GCGTCTCAGCTTACTCAGACT | 1984 | 2587 | 2476 | 111 |
| 702 | LSNMMSV | 1985 | CTGTCTAATATGATGAGTGTT | 1986 | 2586 | 2580 | 7 |
| 703 | MYVAHSS | 1987 | ATGTATGTTGCTCATAGTTCG | 1988 | 2585 | 399 | 2186 |
| 704 | THDPIQR | 1989 | ACTCATGATCCGATTCAGCGT | 1990 | 2585 | 2322 | 263 |
| 705 | HQDRTTL | 1991 | CATCAGGATAGGACGACGCTT | 1992 | 2584 | 2000 | 584 |
| 706 | GTLERTA | 1993 | GGGACGTTGGAACGTACGGCC | 1994 | 2581 | 1389 | 1192 |
| 707 | IVDVTAR | 1995 | ATAGTGGACGTTACTGCTCGG | 1996 | 2580 | 482 | 2098 |
| 708 | IFNTTNT | 1997 | ATTTTTAATACTACGAATACT | 1998 | 2580 | 580 | 2000 |
| 709 | AKLLDSL | 1999 | GCAAAACTCCTCGACAGCCTT | 2000 | 2580 | 1008 | 1572 |
| 710 | WDDSKDR | 2001 | TGGGACGACTCAAAAGACAGA | 2002 | 2579 | 356 | 2223 |
| 711 | MLRGYSQ | 2003 | ATGCTTAGGGGTTATAGTCAG | 2004 | 2578 | 0 | 2578 |
| 712 | QDGMLTR | 2005 | CAAGACGGTATGTTGACAAGG | 2006 | 2578 | 803 | 1775 |
| 713 | LANMLNV | 2007 | CTGGCTAATATGTTGAATGTT | 2008 | 2576 | 0 | 2576 |
| 714 | PYEGAGT | 2009 | CCGTACGAAGGCGCAGGTACT | 2010 | 2576 | 450 | 2126 |
| 715 | MDGKSPP | 2011 | ATGGATGGGAAGTCGCCGCCG | 2012 | 2576 | 2576 | 0 |
| 716 | GQAGTYS | 2013 | GGCCAAGCGGGTACCTACTCG | 2014 | 2575 | 2575 | 0 |
| 717 | MDGKSPT | 2015 | ATGGACGGAAAAAGTCCAACA | 2016 | 2574 | 1016 | 1558 |
| 718 | VMTVETS | 2017 | GTGATGACCGTCGAAACCTCG | 2018 | 2574 | 2250 | 324 |
| 719 | VQMTLHK | 2019 | GTTCAGATGACGCTTCATAAG | 2020 | 2572 | 0 | 2572 |
| 720 | VEWKHPL | 2021 | GTGGAGTGGAAGCATCCTTTG | 2022 | 2571 | 1929 | 642 |
| 721 | RGAESSE | 2023 | CGTGGTGCCGAAAGCAGTGAA | 2024 | 2571 | 571 | 2000 |
| 722 | LSNMLSV | 2025 | CTGTCTAATATGTTGAGTGTT | 2026 | 2570 | 570 | 2000 |
| 723 | ELVATTI | 2027 | GAGCTTGTGGCTACTACTATT | 2028 | 2570 | 2000 | 570 |
| 724 | LAGLGGP | 2029 | CTCGCAGGCCTTGGTGGCCCC | 2030 | 2567 | 373 | 2194 |
| 725 | RISHEGT | 2031 | CGCATATCCCACGAAGGAACT | 2032 | 2567 | 0 | 2567 |
| 726 | TAAGIDR | 2033 | ACTGCTGCGGGGATTGATCGG | 2034 | 2566 | 1487 | 1079 |
| 727 | FAEVAQA | 2035 | TTCGCCGAAGTAGCCCAAGCT | 2036 | 2566 | 0 | 2566 |
| 728 | GPAEGQG | 2037 | GGGCCAGCCGAAGGACAAGGT | 2038 | 2565 | 0 | 2565 |
| 729 | GAADRQI | 2039 | GGCGCAGCAGACCGACAAATA | 2040 | 2565 | 2298 | 266 |
| 730 | AVSGYTV | 2041 | GCAGTGTCAGGGTACACGGTT | 2042 | 2564 | 1754 | 811 |
| 731 | IANLADS | 2043 | ATTGCGAATCTTGCTGATTCG | 2044 | 2564 | 304 | 2260 |
| 732 | TSYDKLV | 2045 | ACGTCGTATGATAAGTTGGTT | 2046 | 2562 | 498 | 2065 |
| 733 | FQDTIGV | 2047 | TTTCAGGATACGATTGGGGTG | 2048 | 2562 | 1161 | 1401 |
| 734 | TNGGEGA | 2049 | ACTAATGGGGGTGAGGGGGCG | 2050 | 2560 | 0 | 2560 |
| 735 | IAQNTPY | 2051 | ATCGCACAAAACACACCCTAC | 2052 | 2558 | 1334 | 1224 |
| 736 | QVHDTKT | 2053 | CAAGTCCACGACACAAAAACG | 2054 | 2558 | 421 | 2136 |
| 737 | RLNEHEA | 2055 | CGTCTGAATGAGCATGAGGCG | 2056 | 2557 | 1782 | 775 |
| 738 | AGSGTEV | 2057 | GCGGGTTCTGGGACTGAGGTT | 2058 | 2557 | 1949 | 608 |
| 739 | IVIAEIH | 2059 | ATTGTGATTGCTGAGATTCAT | 2060 | 2554 | 624 | 1930 |
| 740 | QVRETKT | 2061 | CAAGTTAGGGAAACCAAAACC | 2062 | 2553 | 2226 | 327 |
| 741 | SVNSGLL | 2063 | AGTGTTAATAGTGGGCTGCTT | 2064 | 2551 | 2348 | 203 |
| 742 | VGVNGSH | 2065 | GTGGGTGTGAATGGTTCTCAT | 2066 | 2550 | 2274 | 276 |
| 743 | AAAQSAT | 2067 | GCAGCAGCACAATCGGCAACG | 2068 | 2549 | 2379 | 170 |
| 744 | SKAEGPV | 2069 | TCGAAGGCTGAGGGTCCGGTT | 2070 | 2548 | 2000 | 548 |
| 745 | AGLQVSI | 2071 | GCCGGTTTACAAGTCAGCATC | 2072 | 2547 | 2547 | 0 |
| 746 | QNERITV | 2073 | CAGAATGAGAGGATTACTGTG | 2074 | 2546 | 2000 | 546 |
| 747 | QEKGTST | 2075 | CAAGAAAAAGGAACCTCGACG | 2076 | 2544 | 767 | 1777 |
| 748 | SHGSDSK | 2077 | TCGCACGGCTCCGACTCTAAA | 2078 | 2543 | 2543 | 0 |
| 749 | RMENGNT | 2079 | AGAATGGAAAACGGTAACACC | 2080 | 2542 | 0 | 2542 |
| 750 | LGVEVGA | 2081 | TTGGGTGTGGAGGTTGGGGCG | 2082 | 2542 | 482 | 2060 |
| 751 | TTGPSNA | 2083 | ACTACTGGGCCGAGTAACGCC | 2084 | 2542 | 2000 | 542 |
| 752 | RDLDGKY | 2085 | AGGGACCTTGACGGAAAATAC | 2086 | 2542 | 2542 | 0 |
| 753 | MDTHTNT | 2087 | ATGGACACCCACACAAACACA | 2088 | 2541 | 541 | 2000 |
| 754 | SLINTGS | 2089 | TCACTCATCAACACAGGTTCT | 2090 | 2540 | 540 | 2000 |
| 755 | KSISGEW | 2091 | AAAAGCATCTCTGGCGAATGG | 2092 | 2538 | 2184 | 354 |
| 756 | GVNHAVA | 2093 | GGTGTTAATCATGCGGTGGCT | 2094 | 2538 | 2237 | 301 |
| 757 | AGEHYQA | 2095 | GCAGGCGAACACTACCAAGCG | 2096 | 2538 | 2538 | 0 |
| 758 | TTGLTGS | 2097 | ACGACGGGGCTGACTGGTAGT | 2098 | 2535 | 2000 | 535 |
| 759 | PTQGTLQ | 2099 | CCGACCCAAGGTACCTTGCAA | 2100 | 2535 | 2261 | 274 |
| 760 | GGTQAVL | 2101 | GGTGGGACTCAGGCTGTGCTG | 2102 | 2534 | 2392 | 142 |
| 761 | AADVILN | 2103 | GCTGCCGACGTCATCCTTAAC | 2104 | 2534 | 2534 | 0 |
| 762 | ITTGPGG | 2105 | ATTACGACGGGTCCTGGGGGT | 2106 | 2533 | 1242 | 1290 |
| 763 | TTLAGPA | 2107 | ACTACTCTGGCTGGTCCTGCG | 2108 | 2533 | 2533 | 0 |
| 764 | NNGTLPI | 2109 | AATAATGGTACTTTGCCGATT | 2110 | 2532 | 754 | 1779 |
| 765 | IDSLNSV | 2111 | ATAGACAGTCTGAACTCCGTC | 2112 | 2529 | 2387 | 142 |
| 766 | GAASSTK | 2113 | GGCGCAGCATCGTCCACAAAA | 2114 | 2528 | 0 | 2528 |
| 767 | ELRVKDT | 2115 | GAGCTTAGGGTTAAGGATACT | 2116 | 2528 | 296 | 2232 |
| 768 | MGASATL | 2117 | ATGGGTGCATCCGCAACCTTG | 2118 | 2527 | 2000 | 527 |
| 769 | YGTVVET | 2119 | TACGGAACAGTGGTGGAAACG | 2120 | 2527 | 2000 | 527 |
| 770 | GTLVSEL | 2121 | GGTACGTTGGTGTCGGAGCTG | 2122 | 2522 | 2000 | 522 |
| 771 | ATGTESR | 2123 | GCAACAGGGACCGAATCAAGG | 2124 | 2520 | 305 | 2215 |
| 772 | NGGIGGF | 2125 | AATGGTGGGATTGGTGGTTTT | 2126 | 2520 | 473 | 2047 |
| 773 | LADNHGR | 2127 | CTGGCGGATAATCATGGTAGG | 2128 | 2520 | 520 | 2000 |
| 774 | TSASVSQ | 2129 | ACTTCTGCTTCTGTTTCTCAG | 2130 | 2518 | 518 | 2000 |
| 775 | GNSGGDF | 2131 | GGAAACAGCGGTGGGGACTTC | 2132 | 2518 | 2518 | 0 |
| 776 | TYEDLRV | 2133 | ACTTATGAGGATCTTAGGGTG | 2134 | 2518 | 2518 | 0 |
| 777 | RDASITI | 2135 | CGAGACGCCTCGATAACAATA | 2136 | 2516 | 361 | 2155 |
| 778 | GAQVNGT | 2137 | GGTGCACAAGTAAACGGTACA | 2138 | 2516 | 1595 | 921 |
| 779 | RMTGDLT | 2139 | AGGATGACGGGTGATTTGACT | 2140 | 2514 | 2190 | 324 |
| 780 | ITSEPLP | 2141 | ATCACATCCGAACCCCTACCT | 2142 | 2513 | 0 | 2513 |
| 781 | TLAISEL | 2143 | ACGCTTGCTATCAGCGAATTG | 2144 | 2509 | 2140 | 369 |
| 782 | ASLLNKT | 2145 | GCCAGCTTACTCAACAAAACG | 2146 | 2509 | 2000 | 509 |
| 783 | RDYAEQP | 2147 | CGCGACTACGCTGAACAACCT | 2148 | 2509 | 2382 | 126 |
| 784 | ARVDTGI | 2149 | GCGCGTGTAGACACGGGGATA | 2150 | 2508 | 2000 | 508 |
| 785 | EGKTQLQ | 2151 | GAGGGTAAGACTCAGCTGCAG | 2152 | 2508 | 2508 | 0 |
| 786 | VGNDERP | 2153 | GTTGGGAATGATGAGCGTCCG | 2154 | 2507 | 333 | 2175 |
| 787 | LSLSKDK | 2155 | CTGAGTCTTAGTAAGGATAAG | 2156 | 2507 | 2507 | 0 |
| 788 | ISLDATS | 2157 | ATAAGCCTCGACGCTACATCT | 2158 | 2506 | 2000 | 506 |
| 789 | GTMSPGA | 2159 | GGTACTATGTCTCCTGGGGCT | 2160 | 2506 | 2506 | 0 |
| 790 | GSGERPV | 2161 | GGAAGTGGTGAAAGGCCGGTA | 2162 | 2504 | 2000 | 504 |
| 791 | AGEHYQA | 2163 | GCGGGTGAGCATTATCAGGCT | 2164 | 2504 | 2504 | 0 |
| 792 | RNEGINQ | 2165 | CGTAATGAGGGTATTAATCAG | 2166 | 2502 | 2188 | 314 |
| 793 | GMGASSK | 2167 | GGTATGGGGGCGTCTTCTAAG | 2168 | 2502 | 2389 | 113 |
| 794 | QLVAQDR | 2169 | CAGTTGGTGGCGCAGGATCGG | 2170 | 2500 | 851 | 1649 |
| 795 | TTADIVR | 2171 | ACGACGGCGGATATTGTTAGG | 2172 | 2498 | 332 | 2167 |
| 796 | YTVTGTI | 2173 | TACACCGTAACTGGCACAATC | 2174 | 2498 | 498 | 2000 |
| 797 | GNGTGVL | 2175 | GGTAATGGGACTGGTGTGCTT | 2176 | 2497 | 2363 | 134 |
| 798 | PIHGASS | 2177 | CCAATACACGGGGCGTCATCT | 2178 | 2496 | 2001 | 495 |
| 799 | DSHASGD | 2179 | GATAGTCATGCGTCGGGGGAT | 2180 | 2496 | 2496 | 0 |
| 800 | PNERHTV | 2181 | CCGAATGAGAGGCATACTGTG | 2182 | 2494 | 1581 | 913 |
| 801 | NRLGDRI | 2183 | AACAGGCTGGGCGACCGACTA | 2184 | 2494 | 2322 | 172 |
| 802 | LLQSLND | 2185 | CTCCTACAATCGCTGAACGAC | 2186 | 2494 | 2000 | 494 |
| 803 | DRAELRL | 2187 | GACCGGGCAGAACTAAGGCTT | 2188 | 2493 | 0 | 2493 |
| 804 | RNFSVVL | 2189 | CGGAACTTCAGTGTAGTACTG | 2190 | 2492 | 1945 | 547 |
| 805 | IAGVPQA | 2191 | ATTGCGGGGGTTCCGCAGGCG | 2192 | 2492 | 2491 | 1 |
| 806 | EPSLSSP | 2193 | GAGCCGTCTCTGAGTTCTCCG | 2194 | 2492 | 492 | 2000 |
| 807 | LNGGHVM | 2195 | TTGAATGGGGGTCATGTTATG | 2196 | 2492 | 2171 | 321 |
| 808 | RDLNSDV | 2197 | AGGGACCTTAACTCGGACGTC | 2198 | 2491 | 2274 | 217 |
| 809 | PGQHNQA | 2199 | CCAGGACAACACAACCAAGCC | 2200 | 2490 | 307 | 2183 |
| 810 | GVEGSGM | 2201 | GGGGTGGAAGGCTCCGGAATG | 2202 | 2490 | 1263 | 1227 |
| 811 | QADNNGR | 2203 | CAAGCTGACAACAACGGCCGC | 2204 | 2490 | 2490 | 0 |
| 812 | ADAGIMM | 2205 | GCGGACGCCGGCATCATGATG | 2206 | 2486 | 553 | 1933 |
| 813 | DRADDSR | 2207 | GATAGGGCTGATGATTCTCGT | 2208 | 2486 | 2486 | C |
| 814 | DQTYTSA | 2209 | GACCAAACATACACAAGCGCG | 2210 | 2485 | 2485 | 0 |
| 815 | GVRDTNI | 2211 | GGAGTTCGAGACACAAACATA | 2212 | 2483 | 383 | 2100 |
| 816 | MSVATQR | 2213 | ATGTCAGTCGCGACTCAACGA | 2214 | 2483 | 483 | 2000 |
| 817 | PALEANI | 2215 | CCGGCTCTTGAGGCTAATATT | 2216 | 2482 | 1623 | 858 |
| 818 | VLNEHVA | 2217 | GTCCTTAACGAACACGTAGCT | 2218 | 2482 | 0 | 2482 |
| 819 | GLNEHQA | 2219 | GGTCTGAATGAGCATCAGGCG | 2220 | 2482 | 0 | 2482 |
| 820 | SLDSLTS | 2221 | AGTTTAGACAGCTTAACCAGT | 2222 | 2482 | 2345 | 137 |
| 821 | WTDRESL | 2223 | TGGACTGATCGGGAGTCGCTG | 2224 | 2481 | 2481 | C |
| 822 | DVGTGAL | 2225 | GATGTTGGGACTGGGGCGTTG | 2226 | 2481 | 2481 | 0 |
| 823 | VGHVESP | 2227 | GTAGGCCACGTCGAATCTCCA | 2228 | 2480 | 0 | 2480 |
| 824 | KGSDTAM | 2229 | AAAGGGTCAGACACAGCCATG | 2230 | 2480 | 0 | 2480 |
| 825 | KLSSEKT | 2231 | AAGCTTTCGAGTGAGAAGACT | 2232 | 2478 | 2366 | 112 |
| 826 | VGLSRDL | 2233 | GTTGGGCTGAGTCGGGATCTG | 2234 | 2477 | 0 | 2477 |
| 827 | VSNAVGQ | 2235 | GTTTCGAATGCTGTGGGTCAG | 2236 | 2477 | 2477 | 0 |
| 828 | LSNHGSV | 2237 | TTGAGCAACCACGGATCGGTA | 2238 | 2476 | 394 | 2083 |
| 829 | GAPSLGD | 2239 | GGGGCGCCGTCGTTGGGTGAT | 2240 | 2476 | 2000 | 476 |
| 830 | MNGAHVL | 2241 | ATGAACGGCGCGCACGTATTG | 2242 | 2475 | 2475 | 0 |
| 831 | NGNMASY | 2243 | AACGGAAACATGGCAAGTTAC | 2244 | 2472 | 1620 | 852 |
| 832 | IAQMHSS | 2245 | ATCGCACAAATGCACAGTTCC | 2246 | 2470 | 606 | 1864 |
| 833 | PTTLGHD | 2247 | CCGACAACTCTCGGACACGAC | 2248 | 2470 | 2470 | 0 |
| 834 | QANMLSD | 2249 | CAAGCCAACATGCTCTCAGAC | 2250 | 2469 | 1614 | 856 |
| 835 | WANGNTV | 2251 | TGGGCGAATGGGAATACGGTG | 2252 | 2468 | 2000 | 468 |
| 836 | EEKSASY | 2253 | GAGGAGAAGTCGGCTTCTTAT | 2254 | 2468 | 1731 | 736 |
| 837 | VSPAASV | 2255 | GTTAGTCCTGCTGCGAGTGTT | 2256 | 2468 | 2468 | 0 |
| 838 | ARSLGEV | 2257 | GCGAGGTCGCTTGGGGAGGTT | 2258 | 2467 | 467 | 2000 |
| 839 | MDVSSGP | 2259 | ATGGATGTGAGTAGTGGTCCG | 2260 | 2465 | 2000 | 465 |
| 840 | ISNYTRL | 2261 | ATATCTAACTACACGCGGCTT | 2262 | 2464 | 2000 | 464 |
| 841 | PDERLTV | 2263 | CCTGACGAACGGCTAACGGTT | 2264 | 2463 | 2463 | 0 |
| 842 | TDALKSK | 2265 | ACCGACGCCCTAAAAAGCAAA | 2266 | 2462 | 2462 | 0 |
| 843 | ATDSTQS | 2267 | GCCACCGACAGCACTCAAAGC | 2268 | 2462 | 2462 | 0 |
| 844 | SSLLTTA | 2269 | TCGTCGTTGCTGACTACTGCT | 2270 | 2461 | 603 | 1858 |
| 845 | VLTSPGP | 2271 | GTTCTGACTTCGCCTGGTCCT | 2272 | 2460 | 0 | 2460 |
| 846 | DSHVSGM | 2273 | GATAGTCATGTGTCGGGGATG | 2274 | 2459 | 237 | 2222 |
| 847 | NDSAANS | 2275 | AACGACTCTGCTGCGAACTCC | 2276 | 2459 | 2459 | 0 |
| 848 | ALGVAVA | 2277 | GCTCTTGGGGTTGCTGTTGCT | 2278 | 2458 | 0 | 2458 |
| 849 | GTAGHMS | 2279 | GGGACTGCTGGGCATATGTCG | 2280 | 2457 | 457 | 2000 |
| 850 | NNLGDRL | 2281 | AACAACCTGGGCGACAGGCTC | 2282 | 2456 | 1937 | 519 |
| 851 | LGAGSPN | 2283 | CTAGGCGCCGGAAGCCCGAAC | 2284 | 2456 | 2456 | 0 |
| 852 | SGSNTGT | 2285 | TCGGGGTCTAATACGGGTACT | 2286 | 2455 | 5 | 2450 |
| 853 | GVGASEK | 2287 | GGTGTGGGGGCTAGTGAGAAG | 2288 | 2455 | 472 | 1984 |
| 854 | MSNVGTW | 2289 | ATGAGTAACGTAGGCACATGG | 2290 | 2455 | 2000 | 455 |
| 855 | IVMAENN | 2291 | ATCGTAATGGCGGAAAACAAC | 2292 | 2454 | 0 | 2454 |
| 856 | HVDLGTK | 2293 | CACGTTGACTTAGGCACAAAA | 2294 | 2452 | 224 | 2228 |
| 857 | PNERVTV | 2295 | CCGAATGAGAGGGTTACTGTG | 2296 | 2452 | 2373 | 79 |
| 858 | DRDTNPY | 2297 | GACCGAGACACCAACCCATAC | 2298 | 2452 | 452 | 2000 |
| 859 | DGGLPKS | 2299 | GACGGAGGCTTACCCAAAAGC | 2300 | 2452 | 1419 | 1033 |
| 860 | RISQDGD | 2301 | AGAATATCCCAAGACGGAGAC | 2302 | 2451 | 0 | 2451 |
| 861 | AVLAGTR | 2303 | GCGGTTCTGGCGGGGACTAGG | 2304 | 2450 | 2000 | 450 |
| 862 | KASDTPM | 2305 | AAAGCAAGTGACACGCCCATG | 2306 | 2450 | 2450 | 0 |
| 863 | GNDVGRS | 2307 | GGGAACGACGTAGGCCGCTCG | 2308 | 2446 | 0 | 2446 |
| 864 | STLSGTD | 2309 | TCGACGCTGTCTGGTACTGAT | 2310 | 2446 | 1766 | 680 |
| 865 | FSSEQLT | 2311 | TTTTCGTCTGAGCAGCTTACG | 2312 | 2445 | 0 | 2445 |
| 866 | SHLGDRL | 2313 | AGTCATCTTGGTGATCGTTTG | 2314 | 2443 | 2000 | 444 |
| 867 | AVKEYQS | 2315 | GCTGTTAAAGAATACCAATCT | 2316 | 2443 | 0 | 2443 |
| 868 | MGTPTNT | 2317 | ATGGGTACTCCTACGAATACG | 2318 | 2443 | 0 | 2443 |
| 869 | QSLATGI | 2319 | CAGTCGCTTGCTACTGGGATT | 2320 | 2442 | 921 | 1522 |
| 870 | PGVAMVS | 2321 | CCTGGGGTAGCAATGGTATCT | 2322 | 2442 | 2000 | 442 |
| 871 | SAETRNG | 2323 | TCTGCGGAGACTAGGAATGGG | 2324 | 2441 | 335 | 2106 |
| 872 | EGGYSGR | 2325 | GAGGGTGGTTATAGTGGGCGT | 2326 | 2440 | 2000 | 440 |
| 873 | ETEASSR | 2327 | GAGACGGAGGCGAGTTCGCGT | 2328 | 2440 | 2440 | 0 |
| 874 | STHHTST | 2329 | TCGACGCACCACACCAGTACG | 2330 | 2439 | 2000 | 439 |
| 875 | REMPLSH | 2331 | AGGGAGATGCCTTTGAGTCAT | 2332 | 2438 | 2345 | 93 |
| 876 | RELQSAA | 2333 | CGCGAATTACAAAGCGCAGCT | 2334 | 2437 | 2000 | 437 |
| 877 | GSGSGVL | 2335 | GGTTCTGGGTCGGGGGTGCTG | 2336 | 2437 | 1925 | 512 |
| 878 | GVLTTVT | 2337 | GGAGTCTTGACCACTGTTACG | 2338 | 2433 | 0 | 2433 |
| 879 | NLQGNAH | 2339 | AATCTGCAGGGTAATGCTCAT | 2340 | 2432 | 1160 | 1271 |
| 880 | AAISSQT | 2341 | GCGGCGATTAGTTCTCAGACG | 2342 | 2430 | 0 | 2430 |
| 881 | ETTVSHV | 2343 | GAGACTACGGTTTCTCATGTG | 2344 | 2428 | 1376 | 1052 |
| 882 | ALTNGQR | 2345 | GCACTAACCAACGGTCAACGT | 2346 | 2427 | 2000 | 427 |
| 883 | NNNGATS | 2347 | AATAATAATGGTGCGACTTCT | 2348 | 2426 | 526 | 1900 |
| 884 | IDGKSPP | 2349 | ATTGATGGGAAGTCGCCGCCG | 2350 | 2425 | 0 | 2425 |
| 885 | PTGTVVT | 2351 | CCGACTGGGACTGTTGTTACT | 2352 | 2425 | 425 | 2000 |
| 886 | SGEQLRI | 2353 | TCTGGGGAGCAGCTTAGGATT | 2354 | 2424 | 1343 | 1081 |
| 887 | AVNNVTL | 2355 | GCTGTGAATAATGTTACTCTT | 2356 | 2424 | 2000 | 424 |
| 888 | LSDLMRS | 2357 | CTTAGCGACCTCATGAGGTCT | 2358 | 2424 | 627 | 1797 |
| 889 | QYVVTGG | 2359 | CAGTATGTTGTTACTGGTGGG | 2360 | 2423 | 2000 | 423 |
| 890 | MDGKTPP | 2361 | ATGGACGGTAAAACTCCCCCT | 2362 | 2421 | 0 | 2421 |
| 891 | IGMDPKA | 2363 | ATTGGTATGGATCCGAAGGCG | 2364 | 2420 | 1509 | 910 |
| 892 | GVDAVAY | 2365 | GGAGTGGACGCTGTGGCATAC | 2366 | 2414 | 0 | 2414 |
| 893 | GNQGGTR | 2367 | GGTAATCAGGGGGGGACGCGT | 2368 | 2414 | 587 | 1827 |
| 894 | ASASSPR | 2369 | GCCTCAGCATCATCACCTAGG | 2370 | 2413 | 0 | 2413 |
| 895 | GLSPEAR | 2371 | GGTTTGTCTCCTGAGGCGCGT | 2372 | 2413 | 0 | 2413 |
| 896 | LVTTLHM | 2373 | TTGGTCACCACACTACACATG | 2374 | 2413 | 292 | 2121 |
| 897 | HAGLGII | 2375 | CACGCGGGGCTGGGCATAATC | 2376 | 2412 | 2000 | 412 |
| 898 | AILGASS | 2377 | GCCATACTAGGCGCATCTTCC | 2378 | 2412 | 2000 | 412 |
| 899 | STHDIRV | 2379 | TCTACTCACGACATACGAGTC | 2380 | 2411 | 411 | 2000 |
| 900 | AEQLSHS | 2381 | GCAGAACAACTATCCCACAGC | 2382 | 2411 | 2411 | 0 |
| 901 | LHDTLTR | 2383 | CTGCACGACACATTAACCCGC | 2384 | 2410 | 2000 | 410 |
| 902 | PGEHYPA | 2385 | CCCGGCGAACACTACCCAGCG | 2386 | 2410 | 1755 | 655 |
| 903 | SSGSGVA | 2387 | AGTTCTGGGTCGGGGGTGGCT | 2388 | 2410 | 2410 | 0 |
| 904 | MVDSAQL | 2389 | ATGGTGGATTCGGCGCAGCTT | 2390 | 2407 | 0 | 2407 |
| 905 | SGLVTEL | 2391 | AGCGGATTGGTAACTGAACTG | 2392 | 2406 | 0 | 2406 |
| 906 | HGHIAQS | 2393 | CATGGGCATATTGCGCAGTCG | 2394 | 2406 | 169 | 2237 |
| 907 | HTDGSYV | 2395 | CATACGGATGGGAGTTATGTT | 2396 | 2405 | 254 | 2151 |
| 908 | MAGQPSQ | 2397 | ATGGCGGGTCAGCCTAGTCAG | 2398 | 2404 | 404 | 2000 |
| 909 | PTQGTPR | 2399 | CCTACTCAGGGGACGCCTCGG | 2400 | 2402 | 703 | 1699 |
| 910 | AGAAIVA | 2401 | GCTGGTGCGGCGATTGTTGCG | 2402 | 2402 | 2393 | 9 |
| 911 | VSASTMA | 2403 | GTTAGTGCTTCGACTATGGCT | 2404 | 2401 | 0 | 2401 |
| 912 | SSDSRTL | 2405 | TCGTCGGATTCGCGGACGTTG | 2406 | 2400 | 0 | 2400 |
| 913 | LTDAHGI | 2407 | TTAACGGACGCTCACGGGATC | 2408 | 2398 | 0 | 2398 |
| 914 | TLQGGLS | 2409 | ACTCTGCAGGGTGGTCTGTCT | 2410 | 2398 | 0 | 2398 |
| 915 | SMSSEIW | 2411 | TCGATGTCATCCGAAATATGG | 2412 | 2398 | 226 | 2172 |
| 916 | QYVDTGG | 2413 | CAGTATGTTGATACTGGTGGG | 2414 | 2398 | 2313 | 85 |
| 917 | VSNTSES | 2415 | GTGTCAAACACCTCCGAATCG | 2416 | 2396 | 1370 | 1026 |
| 918 | IGSAGDR | 2417 | ATTGGGAGTGCGGGTGATCGT | 2418 | 2396 | 396 | 2000 |
| 919 | ANEDRMS | 2419 | GCTAATGAGGATCGGATGAGT | 2420 | 2395 | 2000 | 395 |
| 920 | AAESSVR | 2421 | GCGGCGGAGAGTTCTGTGCGG | 2422 | 2395 | 395 | 2000 |
| 921 | SQSDFPN | 2423 | TCACAAAGTGACTTCCCCAAC | 2424 | 2394 | 0 | 2394 |
| 922 | VRGEETV | 2425 | GTTCGTGGGGAAGAAACCGTC | 2426 | 2394 | 985 | 1410 |
| 923 | NNLGDRM | 2427 | AATAATCTTGGTGATCGTATG | 2428 | 2394 | 2000 | 394 |
| 924 | GNLDLKA | 2429 | GGAAACCTTGACCTCAAAGCC | 2430 | 2394 | 752 | 1642 |
| 925 | GTKDVSP | 2431 | GGCACAAAAGACGTGTCACCC | 2432 | 2393 | 2000 | 393 |
| 926 | AIPIAST | 2433 | GCTATTCCGATTGCGTCTACG | 2434 | 2392 | 0 | 2392 |
| 927 | VSASSVD | 2435 | GTATCAGCCTCGTCGGTGGAC | 2436 | 2392 | 2259 | 133 |
| 928 | NMDRDHV | 2437 | AATATGGATCGTGATCATGTG | 2438 | 2390 | 0 | 2390 |
| 929 | SHASDSK | 2439 | TCTCATGCTTCTGATTCGAAG | 2440 | 2390 | 261 | 2130 |
| 930 | PGSDGGH | 2441 | CCGGGGTCGGATGGGGGGCAT | 2442 | 2390 | 1357 | 1033 |
| 931 | NLGEVQM | 2443 | AATTTGGGTGAGGTTCAGATG | 2444 | 2390 | 2390 | 0 |
| 932 | RYFGDAS | 2445 | CGTTACTTCGGAGACGCGAGT | 2446 | 2389 | 2000 | 389 |
| 933 | AADTTVR | 2447 | GCCGCTGACACGACAGTACGC | 2448 | 2388 | 2111 | 276 |
| 934 | VTGVESR | 2449 | GTCACAGGAGTCGAATCTCGA | 2450 | 2387 | 2000 | 387 |
| 935 | ATVSSPR | 2451 | GCGACCGTATCAAGCCCTAGG | 2452 | 2387 | 2000 | 387 |
| 936 | GGNAGLN | 2453 | GGTGGTAATGCGGGGCTTAAT | 2454 | 2387 | 387 | 2000 |
| 937 | RNQSDQM | 2455 | CGGAACCAATCGGACCAAATG | 2456 | 2387 | 2025 | 362 |
| 938 | RHQGTES | 2457 | AGGCACCAAGGAACAGAATCG | 2458 | 2387 | 543 | 1844 |
| 939 | TTSIPTP | 2459 | ACGACGTCTATTCCGACGCCT | 2460 | 2386 | 0 | 2386 |
| 940 | TSHLGQS | 2461 | ACGTCACACCTTGGGCAAAGT | 2462 | 2386 | 2000 | 386 |
| 941 | GVNPAVS | 2463 | GGTGTTAATCCTGCGGTGTCT | 2464 | 2386 | 2000 | 386 |
| 942 | GSDTTVG | 2465 | GGTTCGGATACGACGGTTGGT | 2466 | 2385 | 102 | 2282 |
| 943 | DLAQSGR | 2467 | GATTTGGCTCAGAGTGGGCGT | 2468 | 2385 | 1796 | 589 |
| 944 | GMNEHVA | 2469 | GGGATGAACGAACACGTCGCC | 2470 | 2384 | 283 | 2100 |
| 945 | VMDHKST | 2471 | GTTATGGATCATAAGAGTACG | 2472 | 2384 | 2000 | 384 |
| 946 | MSTDTPA | 2473 | ATGTCGACAGACACGCCCGCG | 2474 | 2381 | 2381 | 0 |
| 947 | AMTVEMP | 2475 | GCTATGACGGTTGAGATGCCT | 2476 | 2380 | 0 | 2380 |
| 948 | GSRENER | 2477 | GGCAGCCGCGAAAACGAACGA | 2478 | 2379 | 1301 | 1078 |
| 949 | ATDSQGL | 2479 | GCTACGGATTCGCAGGGGCTG | 2480 | 2378 | 2378 | 0 |
| 950 | PNERITV | 2481 | CCGAATGAGAGGATTACTGTG | 2482 | 2377 | 2000 | 377 |
| 951 | TTLSDTA | 2483 | ACTACTCTGTCTGATACTGCG | 2484 | 2376 | 0 | 2376 |
| 952 | HSTGAEM | 2485 | CACTCAACAGGCGCCGAAATG | 2486 | 2376 | 0 | 2376 |
| 953 | AVLAAAH | 2487 | GCGGTGTTGGCTGCGGCTCAT | 2488 | 2376 | 2376 | 0 |
| 954 | STLHSST | 2489 | TCTACATTGCACTCGAGCACC | 2490 | 2375 | 2375 | 0 |
| 955 | VLSVGVV | 2491 | GTGCTGAGTGTGGGGGTTGTT | 2492 | 2375 | 2375 | 0 |
| 956 | KEGIATA | 2493 | AAAGAAGGTATCGCTACCGCA | 2494 | 2374 | 2000 | 374 |
| 957 | MVNHTNT | 2495 | ATGGTGAACCACACTAACACT | 2496 | 2374 | 374 | 2000 |
| 958 | ESTATLR | 2497 | GAGTCTACTGCTACTCTTCGG | 2498 | 2372 | 2003 | 369 |
| 959 | AVKEHES | 2499 | GCCGTCAAAGAACACGAATCC | 2500 | 2372 | 2000 | 372 |
| 960 | RYFGDAS | 2501 | CGGTATTTTGGGGATGCTTCG | 2502 | 2371 | 41 | 2330 |
| 961 | LDARGDR | 2503 | CTGGACGCCAGGGGAGACCGT | 2504 | 2371 | 1868 | 503 |
| 962 | PLNKDTR | 2505 | CCATTGAACAAAGACACTCGG | 2506 | 2370 | 648 | 1722 |
| 963 | FQVDKVM | 2507 | TTTCAGGTTGATAAGGTTATG | 2508 | 2369 | 369 | 2000 |
| 964 | LTDKITS | 2509 | CTGACAGACAAAATCACTAGT | 2510 | 2368 | 1629 | 739 |
| 965 | LKPAIEL | 2511 | CTTAAGCCTGCGATTGAGCTG | 2512 | 2368 | 519 | 1849 |
| 966 | ADSVYAK | 2513 | GCTGATAGTGTTTATGCTAAG | 2514 | 2368 | 2274 | 94 |
| 967 | VMSSPGP | 2515 | GTTATGTCTTCGCCTGGTCCT | 2516 | 2367 | 367 | 2000 |
| 968 | ALAISER | 2517 | GCCCTAGCTATCAGTGAACGT | 2518 | 2367 | 2367 | 0 |
| 969 | VTKEHLA | 2519 | GTTACTAAAGAACACCTCGCC | 2520 | 2366 | 1915 | 451 |
| 970 | GLTNTNI | 2521 | GGACTTACAAACACGAACATA | 2522 | 2366 | 2252 | 114 |
| 971 | KQVSMES | 2523 | AAGCAGGTGTCGATGGAGTCG | 2524 | 2365 | 2246 | 119 |
| 972 | SPSASPN | 2525 | AGTCCGTCGGCTTCTCCTAAT | 2526 | 2364 | 2000 | 364 |
| 973 | VVVGNVK | 2527 | GTCGTAGTGGGCAACGTTAAA | 2528 | 2364 | 2000 | 364 |
| 974 | SHGSDTK | 2529 | TCCCACGGAAGTGACACCAAA | 2530 | 2363 | 1823 | 540 |
| 975 | QSREIKI | 2531 | CAGAGTAGGGAGATTAAGATT | 2532 | 2362 | 2362 | 0 |
| 976 | PALQGNI | 2533 | CCGGCTCTTCAGGGTAATATT | 2534 | 2361 | 2361 | 0 |
| 977 | TLVGVER | 2535 | ACACTCGTGGGCGTCGAAAGG | 2536 | 2360 | 0 | 2360 |
| 978 | SGGTMTL | 2537 | AGTGGGGGTACGATGACGCTG | 2538 | 2360 | 0 | 2360 |
| 979 | DRRTDDS | 2539 | GATCGTCGTACTGATGATTCT | 2540 | 2359 | 0 | 2359 |
| 980 | NSDNTRL | 2541 | AATTCGGATAATACTAGGCTG | 2542 | 2357 | 462 | 1896 |
| 981 | ALQSAQV | 2543 | GCACTACAATCTGCACAAGTT | 2544 | 2356 | 2000 | 356 |
| 982 | NVNPDSL | 2545 | AACGTAAACCCCGACAGCTTA | 2546 | 2356 | 2211 | 145 |
| 983 | SNGLPAK | 2547 | AGTAATGGGCTTCCTGCGAAG | 2548 | 2356 | 2205 | 151 |
| 984 | GLNEHVV | 2549 | GGGCTAAACGAACACGTCGTA | 2550 | 2355 | 214 | 2141 |
| 985 | LVTVHTA | 2551 | CTAGTCACCGTACACACGGCA | 2552 | 2354 | 714 | 1640 |
| 986 | GVAATNS | 2553 | GGCGTAGCGGCAACCAACAGC | 2554 | 2354 | 0 | 2354 |
| 987 | QVTDNKT | 2555 | CAAGTAACAGACAACAAAACG | 2556 | 2354 | 199 | 2155 |
| 988 | VNGVSTI | 2557 | GTTAACGGAGTATCCACAATC | 2558 | 2354 | 2169 | 186 |
| 989 | PIASSYE | 2559 | CCTATTGCGTCTAGTTATGAG | 2560 | 2354 | 2354 | 0 |
| 990 | LGESLSR | 2561 | TTGGGTGAGTCTTTGAGTCGG | 2562 | 2354 | 2354 | 0 |
| 991 | PALAGNF | 2563 | CCGGCTCTTGCGGGTAATTTT | 2564 | 2353 | 0 | 2353 |
| 992 | RRDASDP | 2565 | CGGCGAGACGCCTCCGACCCC | 2566 | 2353 | 2353 | 0 |
| 993 | WDNFRFA | 2567 | TGGGACAACTTCAGATTCGCG | 2568 | 2352 | 0 | 2352 |
| 994 | VIGGVLS | 2569 | GTGATTGGTGGTGTGTTGAGT | 2570 | 2352 | 1019 | 1333 |
| 995 | ADDNNRW | 2571 | GCTGATGATAATAATAGGTGG | 2572 | 2352 | 2000 | 352 |
| 996 | LTHSTAV | 2573 | CTTACCCACAGTACAGCGGTG | 2574 | 2352 | 352 | 2000 |
| 997 | VLSGEVL | 2575 | GTCTTGTCTGGAGAAGTCCTT | 2576 | 2351 | 351 | 2000 |
| 998 | SVVADKH | 2577 | AGTGTTGTGGCGGACAAACAC | 2578 | 2350 | 0 | 2350 |
| 999 | PGSTDPK | 2579 | CCTGGGAGTACTGATCCGAAG | 2580 | 2350 | 2000 | 350 |
| 1000 | ASAELGR | 2581 | GCGAGTGCGGAGCTGGGTCGT | 2582 | 2349 | 2218 | 131 |

Mouse_DG

| | | SEQ | | SEQ | | | |
|---|---|---|---|---|---|---|---|
| | | ID | | ID | Combined | BALB/cJ | C57BL/6J |
| Rank | Peptide | NO: | Sequence | NO: | score | score | score |
| 1 | REQQKLW | 2583 | CGTGAGCAGCAGAAGCTTTGG | 2584 | 39151 | 21151 | 18000 |
| 2 | REQQKLW | 2585 | CGGGAACAACAAAAATTATGG | 2586 | 38000 | 22000 | 16000 |
| 3 | ASNPGRW | 2587 | GCTTCGAATCCGGGTCGGTGG | 2588 | 24583 | 4583 | 20000 |
| 4 | SLDKPFK | 2589 | AGTTTGGATAAGCCTTTTAAG | 2590 | 24000 | 0 | 24000 |
| 5 | TLAVPFK | 2591 | ACTTTGGCGGTGCCTTTTAAG | 2592 | 22000 | 0 | 22000 |
| 6 | TLAVPFK | 2593 | ACCTTAGCTGTCCCGTTCAAA | 2594 | 22000 | 0 | 22000 |
| 7 | SLDKPFK | 2595 | TCACTGGACAAACCATTCAAA | 2596 | 21590 | 0 | 21590 |
| 8 | WTLESGH | 2597 | TGGACTCTGGAGTCTGGTCAT | 2598 | 20534 | 4228 | 16306 |
| 9 | ASNPGRW | 2599 | GCAAGCAACCCTGGAAGATGG | 2600 | 18000 | 0 | 18000 |
| 10 | REQKKLW | 2601 | CGTGAGCAGAAGAAGCTTTGG | 2602 | 17695 | 10175 | 7520 |
| 11 | WTLESGH | 2603 | TGGACGCTCGAATCGGGCCAC | 2604 | 17407 | 4000 | 13407 |
| 12 | REQKKLW | 2605 | CGGGAACAAAAAAAACTGTGG | 2606 | 16457 | 7319 | 9138 |
| 13 | ERLLVQL | 2607 | GAGCGGCTGCTTGTTCAGCTG | 2608 | 15513 | 2000 | 13513 |
| 14 | RMQRTLY | 2609 | CGTATGCAGCGGACTCTGTAT | 2610 | 12453 | 2000 | 10453 |
| 15 | ERLLVQL | 2611 | GAACGACTTCTAGTCCAACTA | 2612 | 11949 | 0 | 11949 |
| 16 | LGFSPPR | 2613 | CTTGGATTCTCACCTCCCCGT | 2614 | 11710 | 0 | 11710 |
| 17 | WAISDGY | 2615 | TGGGCGATTAGTGATGGGTAT | 2616 | 10828 | 6571 | 4258 |
| 18 | TEKLPFR | 2617 | ACTGAGAAGCTGCCTTTTCGG | 2618 | 10129 | 0 | 10129 |
| 19 | VRGSSIL | 2619 | GTGCGTGGGTCGTCGATTCTT | 2620 | 9714 | 7617 | 2097 |
| 20 | KLADSVP | 2621 | AAACTTGCAGACTCAGTGCCC | 2622 | 9356 | 7144 | 2212 |
| 21 | VRGSSIL | 2623 | GTCCGGGGATCCAGTATCCTG | 2624 | 8449 | 5059 | 3390 |
| 22 | RQQQKLW | 2625 | AGGCAACAACAAAAATTATGG | 2626 | 8393 | 2000 | 6393 |
| 23 | TVNNDRF | 2627 | ACCGTTAACAACGACCGATTC | 2628 | 8028 | 5611 | 2417 |
| 24 | TVSENAV | 2629 | ACGGTTAGTGAGAATGCGGTT | 2630 | 8000 | 8000 | 0 |
| 25 | MSANERT | 2631 | ATGTCGGCGAATGAGCGTACG | 2632 | 8000 | 6000 | 2000 |
| 26 | RDQQKLW | 2633 | CGTGATCAGCAGAAGCTTTGG | 2634 | 7761 | 4813 | 2948 |
| 27 | GASNGGT | 2635 | GGGGCGAGTAATGGTGGGACT | 2636 | 7674 | 5252 | 2422 |
| 28 | TEKLPFR | 2637 | ACAGAAAAACTCCCCTTCAGA | 2638 | 7548 | 0 | 7548 |
| 29 | ASTATLW | 2639 | GCCTCAACGGCCACCTTATGG | 2640 | 6807 | 0 | 6807 |
| 30 | VTELTKF | 2641 | GTGACTGAGCTTACGAAGTTT | 2642 | 6655 | 4000 | 2655 |
| 31 | KEISVSV | 2643 | AAGGAGATTAGTGTGTCGGTT | 2644 | 6420 | 6007 | 413 |
| 32 | QVAQQGA | 2645 | CAGGTTGCGCAGCAGGGGGCG | 2646 | 6393 | 2000 | 4393 |
| 33 | GVAGTNT | 2647 | GGGGTGGCTGGGACGAATACT | 2648 | 6386 | 6011 | 375 |
| 34 | ASAQGAL | 2649 | GCGTCTGCTCAGGGTGCGCTT | 2650 | 6382 | 1444 | 4938 |
| 35 | QSVDRSK | 2651 | CAGTCGGTGGATCGTAGTAAG | 2652 | 6293 | 5197 | 1096 |
| 36 | RYVGESS | 2653 | CGGTACGTCGGAGAAAGCAGT | 2654 | 6252 | 1422 | 4830 |
| 37 | LGHNAGV | 2655 | TTGGGGCATAATGCTGGTGTT | 2656 | 6239 | 2003 | 4237 |
| 38 | RAAGTSA | 2657 | CGCGCCGCTGGCACCTCAGCA | 2658 | 6000 | 6000 | 0 |
| 39 | VGISSGV | 2659 | GTTGGTATTTCTTCGGGTGTG | 2660 | 6000 | 4000 | 2000 |
| 40 | VLVSPGP | 2661 | GTTCTGGTTTCGCCTGGTCCT | 2662 | 6000 | 4000 | 2000 |
| 41 | SGETLRI | 2663 | TCTGGGGAGACGCTTAGGATT | 2664 | 5977 | 4000 | 1977 |
| 42 | STEGAAL | 2665 | AGTACGGAGGGGGCGGCTCTG | 2666 | 5954 | 5715 | 239 |
| 43 | SSSGAAR | 2667 | TCTTCTTCAGGTGCCGCCCGC | 2668 | 5926 | 5926 | 0 |
| 44 | VLASNGP | 2669 | GTGTTAGCGTCCAACGGGCCG | 2670 | 5898 | 3269 | 2628 |
| 45 | VVQVTGR | 2671 | GTTGTTCAGGTTACTGGGCGT | 2672 | 5875 | 5410 | 465 |
| 46 | FAVRLSS | 2673 | TTTGCTGTGCGGTTGTCGTCG | 2674 | 5855 | 2809 | 3046 |
| 47 | LVRDTKT | 2675 | CTCGTAAGAGACACAAAAACG | 2676 | 5811 | 5452 | 359 |
| 48 | SGESLSR | 2677 | TCTGGCGAAAGCTTATCTAGG | 2678 | 5804 | 3804 | 2000 |
| 49 | TLANSQR | 2679 | ACTTTGGCGAATTCTCAGCGG | 2680 | 5746 | 4000 | 1746 |
| 50 | SQEQRAR | 2681 | AGTCAGGAGCAGAGGGCTCGT | 2682 | 5679 | 4000 | 1679 |
| 51 | SREGGNV | 2683 | AGCCGAGAAGGAGGGAACGTA | 2684 | 5580 | 3580 | 2000 |
| 52 | LGGSSMG | 2685 | CTTGGTGGGTCTTCAATGGGG | 2686 | 5565 | 2612 | 2953 |
| 53 | SGSTDKL | 2687 | TCTGGTTCGACTGATAAGTTG | 2688 | 5556 | 3264 | 2292 |
| 54 | RDQQKLW | 2689 | CGGGACCAACAAAAACTGTGG | 2690 | 5404 | 4000 | 1404 |
| 55 | RVSEVGS | 2691 | CGCGTCTCAGAAGTTGGCAGC | 2692 | 5379 | 5379 | 0 |
| 56 | LGFSPPR | 2693 | CTGGGTTTTAGTCCGCCGAGG | 2694 | 5369 | 0 | 5369 |
| 57 | SPADTRR | 2695 | TCTCCTGCGGATACTAGGAGG | 2696 | 5338 | 5338 | 0 |
| 58 | LSRGDEM | 2697 | TTGAGTCGCGGAGACGAAATG | 2698 | 5326 | 0 | 5326 |
| 59 | RMQRTLY | 2699 | CGAATGCAACGAACATTGTAC | 2700 | 5315 | 2000 | 3315 |
| 60 | IANLAAS | 2701 | ATTGCGAATCTTGCTGCTTCG | 2702 | 5311 | 5107 | 204 |
| 61 | AGGVRDR | 2703 | GCGGGTGGTGTTCGTGATCGT | 2704 | 5299 | 4717 | 582 |
| 62 | GSGSGGL | 2705 | GGGAGTGGATCCGGAGGCTTA | 2706 | 5252 | 5073 | 179 |
| 63 | TLANSER | 2707 | ACACTCGCCAACTCAGAAAGG | 2708 | 5237 | 2000 | 3237 |
| 64 | VNYSVAL | 2709 | GTTAATTATTCGGTGGCGCTT | 2710 | 5206 | 3287 | 1920 |
| 65 | KNPGVYT | 2711 | AAGAATCCGGGGGTGTATACT | 2712 | 5173 | 1750 | 3423 |
| 66 | QREAARI | 2713 | CAGCGTGAGGCGGCGCGGATT | 2714 | 5173 | 1534 | 3639 |
| 67 | LSQGSQQ | 2715 | CTATCCCAAGGTAGTCAACAA | 2716 | 5167 | 5167 | 0 |
| 68 | NGSEGDR | 2717 | AACGGTTCGGAAGGGGACAGG | 2718 | 5157 | 3157 | 2000 |
| 69 | NVGVVQL | 2719 | AACGTAGGCGTAGTACAACTA | 2720 | 5144 | 4780 | 364 |
| 70 | TLAVPF* | 2721 | ACTTTGGCGGTGCCTTTTTAA | 2722 | 5135 | 0 | 5135 |
| 71 | HSLQTSA | 2723 | CACTCATTACAAACCTCTGCG | 2724 | 5096 | 2682 | 2414 |
| 72 | SATDVKH | 2725 | TCGGCAACAGACGTAAAACAC | 2726 | 5083 | 5083 | 0 |
| 73 | SGANLSY | 2727 | TCTGGTGCGAATTTGTCTTAT | 2728 | 4985 | 4271 | 714 |
| 74 | KAHDGEV | 2729 | AAAGCGCACGACGGCGAAGTT | 2730 | 4978 | 2000 | 2978 |
| 75 | KASDTPM | 2731 | AAGGCTTCTGATACTCCTATG | 2732 | 4840 | 2000 | 2840 |
| 76 | PGEHNKA | 2733 | CCGGGTGAGCATAATAAGGCT | 2734 | 4838 | 4743 | 95 |
| 77 | NSAADRQ | 2735 | AATAGTGCGGCTGATCGTCAG | 2736 | 4808 | 2000 | 2808 |
| 78 | FTHGTGT | 2737 | TTCACGCACGGCACAGGCACA | 2738 | 4789 | 4000 | 789 |
| 79 | LSNHGPI | 2739 | CTCTCCAACCACGGCCCGATC | 2740 | 4759 | 3814 | 945 |
| 80 | GGASPVR | 2741 | GGGGGTGCTTCTCCTGTGCGG | 2742 | 4756 | 4756 | 0 |
| 81 | KLTNIGT | 2743 | AAACTTACCAACATAGGCACG | 2744 | 4723 | 2000 | 2723 |
| 82 | PGSDGRT | 2745 | CCAGGAAGTGACGGAAGGACG | 2746 | 4719 | 4000 | 719 |
| 83 | AGGGATR | 2747 | GCCGGTGGAGGCGCCACTCGC | 2748 | 4718 | 0 | 4718 |
| 84 | GTGSTIV | 2749 | GGTACAGGCTCCACAATCGTA | 2750 | 4716 | 2942 | 1774 |
| 85 | SNGAGYL | 2751 | TCCAACGGAGCTGGGTACTTA | 2752 | 4714 | 2000 | 2714 |
| 86 | GGTSSGH | 2753 | GGAGGAACTTCCAGCGGGCAC | 2754 | 4703 | 4000 | 703 |
| 87 | AVLSQNI | 2755 | GCTGTGTTGTCTCAGAATATT | 2756 | 4696 | 3737 | 959 |
| 88 | REEQKVW | 2757 | CGTGAGGAGCAGAAGGTTTGG | 2758 | 4694 | 4000 | 694 |
| 89 | PTQGTNR | 2759 | CCCACACAAGGAACCAACCGC | 2760 | 4690 | 4000 | 690 |
| 90 | VTTVSNV | 2761 | GTTACGACCGTGAGCAACGTT | 2762 | 4685 | 2000 | 2685 |
| 91 | GNVNGGA | 2763 | GGTAACGTGAACGGAGGAGCG | 2764 | 4685 | 0 | 4685 |
| 92 | SVDSGRL | 2765 | TCGGTCGACTCTGGACGTTTG | 2766 | 4679 | 4000 | 679 |
| 93 | LSNHVPV | 2767 | TTATCCAACCACGTTCCGGTG | 2768 | 4648 | 2000 | 2648 |
| 94 | ASTATLW | 2769 | GCGTCTACTGCTACTCTTTGG | 2770 | 4647 | 1574 | 3073 |
| 95 | SDKPVNT | 2771 | TCCGACAAACCAGTCAACACA | 2772 | 4643 | 3103 | 1540 |
| 96 | NVQTVST | 2773 | AACGTTCAAACCGTCTCAACT | 2774 | 4642 | 4000 | 642 |
| 97 | RTSTDVV | 2775 | AGAACTTCCACAGACGTGGTA | 2776 | 4611 | 4000 | 611 |
| 98 | KASDTPK | 2777 | AAAGCGTCCGACACACCGAAA | 2778 | 4602 | 4474 | 127 |
| 99 | VQGPQNG | 2779 | GTGCAGGGTCCGCAGAATGGT | 2780 | 4600 | 2000 | 2600 |
| 100 | GGTNSGH | 2781 | GGCGGAACCAACAGCGGCCAC | 2782 | 4599 | 4000 | 599 |
| 101 | GNSGGRF | 2783 | GGGAATAGTGGGGGTCGTTTT | 2784 | 4598 | 2000 | 2598 |
| 102 | LAGLGGG | 2785 | CTAGCTGGGCTGGGTGGCGGG | 2786 | 4578 | 2000 | 2578 |
| 103 | QLRDTKT | 2787 | CAGCTGCGTGATACTAAGACT | 2788 | 4577 | 3332 | 1245 |
| 104 | MGVAGVH | 2789 | ATGGGTGTTGCTGGAGTTCAC | 2790 | 4572 | 2000 | 2572 |
| 105 | AVPGTYS | 2791 | GCGGTGCCTGGGACGTATTCT | 2792 | 4567 | 4000 | 567 |
| 106 | MAAKSTP | 2793 | ATGGCTGCGAAGTCGACGCCG | 2794 | 4558 | 3666 | 892 |
| 107 | QVRDTNT | 2795 | CAGGTGCGTGATACTAATACT | 2796 | 4555 | 3848 | 707 |
| 108 | LHNLTQD | 2797 | CTTCATAATCTTACGCAGGAT | 2798 | 4537 | 2611 | 1926 |
| 109 | PGNSASI | 2799 | CCAGGAAACTCCGCATCGATA | 2800 | 4515 | 4133 | 382 |
| 110 | MGGGTNH | 2801 | ATGGGAGGTGGGACCAACCAC | 2802 | 4508 | 4508 | 0 |
| 111 | TLANSQR | 2803 | ACACTAGCCAACAGTCAACGT | 2804 | 4497 | 2000 | 2497 |
| 112 | SAETLRL | 2805 | TCTGCGGAGACGCTTAGGCTT | 2806 | 4491 | 4000 | 491 |
| 113 | MGGGANP | 2807 | ATGGGGGGGGGTGCTAATCCG | 2808 | 4488 | 4000 | 488 |
| 114 | GLAETRA | 2809 | GGGCTTGCTGAGACTAGGGCT | 2810 | 4468 | 2000 | 2468 |
| 115 | AMSSTVG | 2811 | GCAATGAGTTCCACCGTTGGC | 2812 | 4461 | 4000 | 461 |
| 116 | TGPQVSI | 2813 | ACTGGGCCGCAGGTTAGTATT | 2814 | 4457 | 2000 | 2457 |
| 117 | SVDNGKR | 2815 | TCGGTGGATAATGGGAAGCGG | 2816 | 4453 | 2875 | 1578 |
| 118 | NVSRDHS | 2817 | AACGTCTCCCGTGACCACAGT | 2818 | 4446 | 4000 | 446 |
| 119 | KSNSENS | 2819 | AAGTCGAATTCGGAGAATAGT | 2820 | 4431 | 2000 | 2431 |
| 120 | VSLNEGH | 2821 | GTGTCGCTTAATGAGGGGCAT | 2822 | 4422 | 2000 | 2422 |
| 121 | VTADRTT | 2823 | GTGACTGCTGATCGGACTACG | 2824 | 4397 | 3818 | 579 |
| 122 | VLGGTAG | 2825 | GTGCTGGGTGGTACTGCGGGG | 2826 | 4385 | 2000 | 2385 |
| 123 | TAVNSTS | 2827 | ACGGCTGTGAATTCGACTTCG | 2828 | 4385 | 1947 | 2438 |
| 124 | LSLTDVV | 2829 | CTGAGTTTGACTGATGTGGTT | 2830 | 4384 | 3943 | 441 |
| 125 | PIHGASS | 2831 | CCGATTCATGGTGCTAGTTCG | 2832 | 4378 | 4000 | 378 |
| 126 | GINAVGP | 2833 | GGGATTAATGCTGTTGGTCCG | 2834 | 4370 | 2000 | 2370 |
| 127 | GSGTGVA | 2835 | GGTTCTGGGACGGGGGTGGCT | 2836 | 4367 | 0 | 4367 |
| 128 | RQQQKLW | 2837 | CGTCAGCAGCAGAAGCTTTGG | 2838 | 4358 | 1959 | 2399 |
| 129 | TGQTEMT | 2839 | ACGGGGCAGACTGAGATGACT | 2840 | 4341 | 2000 | 2341 |
| 130 | VGNSSGV | 2841 | GTGGGCAACTCCTCTGGGGTT | 2842 | 4334 | 2000 | 2334 |
| 131 | FNSGTGT | 2843 | TTCAACAGCGGGACTGGCACA | 2844 | 4333 | 4000 | 333 |
| 132 | KLAEGIR | 2845 | AAGCTTGCTGAGGGGATTAGG | 2846 | 4328 | 0 | 4328 |
| 133 | GNQGGTR | 2847 | GGTAATCAGGGGGGGACGCGT | 2848 | 4320 | 4000 | 320 |
| 134 | GSESGVA | 2849 | GGTTCTGAGTCGGGGGTGGCT | 2850 | 4316 | 4000 | 316 |
| 135 | TSSRPEE | 2851 | ACTTCTTCTCGGCCGGAGGAG | 2852 | 4312 | 4312 | 0 |
| 136 | RSVGTSA | 2853 | CGTTCGGTGGGTACTTCGGCG | 2854 | 4312 | 4000 | 312 |
| 137 | AVVLNAL | 2855 | GCGGTGGTGCTGAATGCTTTG | 2856 | 4302 | 4000 | 302 |
| 138 | RTSTDVV | 2857 | AGGACGAGTACGGATGTTGTG | 2858 | 4300 | 4300 | 0 |
| 139 | LRNTQLD | 2859 | TTGCGGAATACTCAGTTGGAT | 2860 | 4284 | 2000 | 2284 |
| 140 | IPPGVPR | 2861 | ATTCCGCCTGGGGTTCCGCGT | 2862 | 4280 | 3607 | 673 |
| 141 | MNGGHIL | 2863 | ATGAATGGGGGTCATATTCTG | 2864 | 4268 | 1339 | 2930 |
| 142 | RYVGESS | 2865 | AGGTATGTGGGGGAGTCTTCG | 2866 | 4251 | 2642 | 1610 |
| 143 | PNGVSVV | 2867 | CCTAATGGTGTTTCTGTGGTG | 2868 | 4247 | 3597 | 650 |
| 144 | ASLGAYS | 2869 | GCGTCGCTTGGGGCGTATTCG | 2870 | 4244 | 3212 | 1032 |
| 145 | GSGSVVA | 2871 | GGTTCTGGGTCGGTGGTGGCT | 2872 | 4238 | 959 | 3279 |
| 146 | MGSNGQV | 2873 | ATGGGGTCTAATGGGCAGGTT | 2874 | 4236 | 2000 | 2236 |
| 147 | TGVLINS | 2875 | ACGGGTGTTTTGATTAATTCG | 2876 | 4228 | 1472 | 2756 |
| 148 | LSLTGGV | 2877 | TTGTCGCTCACCGGGGGAGTC | 2878 | 4208 | 1210 | 2998 |
| 149 | SPEGRNV | 2879 | TCGCCCGAAGGCCGCAACGTA | 2880 | 4195 | 4000 | 195 |
| 150 | TIPNLSR | 2881 | ACAATACCGAACTTATCGCGC | 2882 | 4184 | 2911 | 1273 |
| 151 | KNGGHVQ | 2883 | AAAAACGGTGGGCACGTACAA | 2884 | 4180 | 4000 | 180 |
| 152 | ADLAGSR | 2885 | GCGGATCTGGCGGGGTCTAGG | 2886 | 4173 | 4000 | 173 |
| 153 | SYGSDSK | 2887 | TCTTATGGTTCTGATTCGAAG | 2888 | 4164 | 2830 | 1334 |
| 154 | SLGADVG | 2889 | AGTTTGGGGGCGGATGTTGGG | 2890 | 4164 | 2000 | 2164 |
| 155 | TVLTGSF | 2891 | ACCGTCCTCACCGGAAGCTTC | 2892 | 4136 | 4000 | 136 |
| 156 | GNSGGRF | 2893 | GGCAACTCAGGCGGCAGGTTC | 2894 | 4116 | 2000 | 2116 |
| 157 | DRGGSTV | 2895 | GACCGTGGGGGGTCCACCGTA | 2896 | 4104 | 1882 | 2222 |
| 158 | AGYSGTT | 2897 | GCAGGTTACTCCGGTACGACG | 2898 | 4104 | 1803 | 2301 |
| 159 | KGSDSPM | 2899 | AAGGGTTCTGATTCTCCTATG | 2900 | 4085 | 0 | 4085 |
| 160 | DSHVSGY | 2901 | GACTCGCACGTATCAGGCTAC | 2902 | 4079 | 0 | 4079 |
| 161 | KSHSEYS | 2903 | AAGTCGCATTCGGAGTATAGT | 2904 | 4071 | 2000 | 2071 |
| 162 | PTQGTHP | 2905 | CCTACTCAGGGGACGCATCCG | 2906 | 4070 | 2874 | 1196 |
| 163 | LEQSVAR | 2907 | CTCGAACAATCTGTCGCACGC | 2908 | 4058 | 4058 | 0 |
| 164 | KEIRASV | 2909 | AAGGAGATTCGTGCGTCGGTT | 2910 | 4056 | 3266 | 790 |
| 165 | NAINRMV | 2911 | AATGCGATTAATCGTATGGTG | 2912 | 4030 | 1095 | 2935 |
| 166 | KIGENAS | 2913 | AAAATCGGCGAAAACGCGAGT | 2914 | 4011 | 2000 | 2011 |
| 167 | KMGGGVS | 2915 | AAGATGGGTGGTGGGGTGTCG | 2916 | 4007 | 4007 | 0 |
| 168 | EVGNVSR | 2917 | GAGGTGGGGAATGTGTCTCGT | 2918 | 4002 | 3303 | 699 |
| 169 | RVTTHTQ | 2919 | CGTGTTACTACTCATACGCAG | 2920 | 4000 | 4000 | 0 |
| 170 | AGFSNQT | 2921 | GCCGGATTCTCGAACCAAACT | 2922 | 4000 | 4000 | 0 |
| 171 | QREAARI | 2923 | CAAAGGGAAGCTGCACGCATC | 2924 | 4000 | 4000 | 0 |
| 172 | LSLNTKT | 2925 | CTATCCCTAAACACCAAAACC | 2926 | 4000 | 4000 | 0 |
| 173 | RGGVSSV | 2927 | CGTGGTGGAGTTTCTAGTGTT | 2928 | 4000 | 4000 | 0 |
| 174 | ENRVNNA | 2929 | GAAAACAGAGTGAACAACGCA | 2930 | 4000 | 4000 | 0 |
| 175 | SNGGRVE | 2931 | AGTAACGGCGGACGCGTTGAA | 2932 | 4000 | 4000 | 0 |
| 176 | GVKNTNI | 2933 | GGCGTCAAAAACACGAACATC | 2934 | 4000 | 4000 | 0 |
| 177 | SDRTHAS | 2935 | TCGGATCGGACTCATGCGTCG | 2936 | 4000 | 4000 | 0 |
| 178 | GSGSGVL | 2937 | GGTTCTGGGTCGGGGGTGCTG | 2938 | 4000 | 4000 | 0 |
| 179 | AEGTDGV | 2939 | GCCGAAGGGACGGACGGCGTC | 2940 | 4000 | 4000 | 0 |
| 180 | GGKGEGP | 2941 | GGTGGGAAGGGTGAGGGTCCG | 2942 | 4000 | 4000 | 0 |
| 181 | TAVKVSG | 2943 | ACGGCTGTGAAGGTTAGTGGT | 2944 | 4000 | 4000 | 0 |
| 182 | TTGEGIR | 2945 | ACTACGGGTGAGGGTATTCGT | 2946 | 4000 | 4000 | 0 |
| 183 | LLASGAK | 2947 | CTGCTTGCGAGTGGGGCTAAG | 2948 | 4000 | 4000 | 0 |
| 184 | SLGTMTL | 2949 | TCTCTTGGAACCATGACCCTC | 2950 | 4000 | 4000 | 0 |
| 185 | RSEGTSA | 2951 | CGTTCGGAGGGTACTTCGGCG | 2952 | 4000 | 4000 | 0 |
| 186 | TNGQASR | 2953 | ACTAATGGTCAGGCGTCTAGG | 2954 | 4000 | 4000 | 0 |
| 187 | LGHKAGG | 2955 | TTGGGGCATAAGGCTGGTGGT | 2956 | 4000 | 4000 | 0 |
| 188 | VVAGTYS | 2957 | GTGGTGGCTGGGACGTATTCT | 2958 | 4000 | 4000 | 0 |
| 189 | SGEQIRL | 2959 | TCTGGGGAGCAGATTAGGCTT | 2960 | 4000 | 4000 | 0 |
| 190 | RGGVTGE | 2961 | CGGGGGGGTGTTACTGGTGAG | 2962 | 4000 | 4000 | 0 |
| 191 | VSSEAPL | 2963 | GTGTCTTCGGAGGCGCCGCTG | 2964 | 4000 | 4000 | 0 |
| 192 | RGPDHKT | 2965 | AGAGGCCCCGACCACAAAACT | 2966 | 4000 | 4000 | 0 |
| 193 | QAHGGPR | 2967 | CAGGCGCATGGGGGGCCTCGT | 2968 | 4000 | 4000 | 0 |
| 194 | LLTDKRV | 2969 | CTTCTTACTGATAAGCGTGTG | 2970 | 4000 | 4000 | 0 |
| 195 | TSPGAGL | 2971 | ACTTCTCCGGGTGCGGGGCTG | 2972 | 4000 | 2000 | 2000 |
| 196 | LTDSRPV | 2973 | TTGACGGATAGTAGGCCTGTT | 2974 | 4000 | 2000 | 2000 |
| 197 | RSEVNGV | 2975 | CGTTCCGAAGTAAACGGTGTG | 2976 | 4000 | 2000 | 2000 |
| 198 | SPGLSIS | 2977 | AGTCCTGGGCTGTCTATTAGT | 2978 | 4000 | 2000 | 2000 |
| 199 | SGSLVGA | 2979 | TCAGGGAGCCTAGTTGGTGCC | 2980 | 4000 | 2000 | 2000 |
| 200 | MVDKPSE | 2981 | ATGGTTGATAAGCCTTCTGAG | 2982 | 4000 | 2000 | 2000 |
| 201 | TPSAFPN | 2983 | ACTCCGTCGGCTTTTCCTAAT | 2984 | 4000 | 2000 | 2000 |
| 202 | MSLNDGV | 2985 | ATGAGTTTGAATGATGGGGTT | 2986 | 4000 | 2000 | 2000 |
| 203 | TTEAIVR | 2987 | ACTACTGAAGCGATCGTCCGC | 2988 | 4000 | 2000 | 2000 |
| 204 | SLVNASF | 2989 | AGTCTGGTTAATGCTTCGTTT | 2990 | 4000 | 2000 | 2000 |
| 205 | LSNHRPV | 2991 | CTAAGCAACCACCGACCAGTG | 2992 | 4000 | 2000 | 2000 |
| 206 | GSGSGVL | 2993 | GGCTCGGGATCAGGTGTACTC | 2994 | 4000 | 2000 | 2000 |
| 207 | AMSSTVG | 2995 | GCGATGTCGAGTACTGTGGGT | 2996 | 4000 | 2000 | 2000 |
| 208 | GMVTNHV | 2997 | GGCATGGTTACTAACCACGTT | 2998 | 4000 | 2000 | 2000 |
| 209 | KMGGGVS | 2999 | AAAATGGGCGGGGGTGTTAGC | 3000 | 4000 | 2000 | 2000 |
| 210 | KSDRGVV | 3001 | AAGTCGGATCGTGGGGTTGTT | 3002 | 4000 | 2000 | 2000 |
| 211 | QLRDTKT | 3003 | CAACTTCGCGACACGAAAACG | 3004 | 4000 | 2000 | 2000 |
| 212 | VANGFPR | 3005 | GTCGCTAACGGCTTCCCCAGA | 3006 | 4000 | 2000 | 2000 |
| 213 | YVNGATE | 3007 | TATGTGAATGGGGCGACTGAG | 3008 | 4000 | 0 | 4000 |
| 214 | QVTDNKT | 3009 | CAAGTCACCGACAACAAAACG | 3010 | 4000 | 0 | 4000 |
| 215 | PGHGPER | 3011 | CCTGGTCATGGTCCGGAGAGG | 3012 | 4000 | 0 | 4000 |
| 216 | MLGQAGG | 3013 | ATGCTGGGTCAGGCTGGGGGG | 3014 | 4000 | 0 | 4000 |
| 217 | APDSTVR | 3015 | GCGCCGGATAGTACTGTGCGG | 3016 | 4000 | 0 | 4000 |
| 218 | SHSSDSK | 3017 | TCTCATAGTTCTGATTCGAAG | 3018 | 4000 | 0 | 4000 |
| 219 | GLHGQSA | 3019 | GGCCTCCACGGACAATCCGCC | 3020 | 4000 | 0 | 4000 |
| 220 | NLGVGQM | 3021 | AACTTAGGAGTAGGCCAAATG | 3022 | 4000 | 0 | 4000 |
| 221 | FISSTMR | 3023 | TTTATTTCTAGTACGATGCGT | 3024 | 4000 | 0 | 4000 |
| 222 | DGTQSGR | 3025 | GACGGCACACAATCCGGCAGG | 3026 | 4000 | 0 | 4000 |
| 223 | MAGKSSP | 3027 | ATGGCGGGAAAAAGTTCTCCA | 3028 | 3990 | 2000 | 1990 |
| 224 | LSTGAQM | 3029 | CTTTCGACGGGTGCGCAGATG | 3030 | 3989 | 1979 | 2010 |
| 225 | NGGSEKR | 3031 | AATGGTGGTTCTGAGAAGCGT | 3032 | 3981 | 3293 | 688 |
| 226 | EDRSTTP | 3033 | GAGGATCGGTCGACTACTCCT | 3034 | 3974 | 3974 | 0 |
| 227 | GSVSSTK | 3035 | GGCTCGGTATCCTCAACAAAA | 3036 | 3974 | 3974 | 0 |
| 228 | KNPGVDP | 3037 | AAGAATCCGGGGGTGGATCCT | 3038 | 3961 | 3476 | 485 |
| 229 | SPSALPN | 3039 | TCACCCTCAGCCCTCCCGAAC | 3040 | 3959 | 2000 | 1959 |
| 230 | LPSLTGG | 3041 | CTTCCGAGTTTGACTGGGGGT | 3042 | 3958 | 943 | 3015 |
| 231 | TDRSDKG | 3043 | ACTGATAGGTCTGATAAGGGG | 3044 | 3956 | 1844 | 2112 |
| 232 | AQQGSTL | 3045 | GCTCAGCAGGGGTCTACGCTG | 3046 | 3953 | 1953 | 2000 |
| 233 | VLESNPR | 3047 | GTGCTTGAGTCGAATCCGCGG | 3048 | 3952 | 3385 | 567 |
| 234 | RIVGSDP | 3049 | AGGATTGTGGGTAGTGATCCG | 3050 | 3952 | 0 | 3952 |
| 235 | LSMTHGV | 3051 | CTGAGTATGACTCATGGGGTT | 3052 | 3948 | 6 | 3942 |
| 236 | KLAERIR | 3053 | AAACTCGCGGAACGTATCCGG | 3054 | 3943 | 3711 | 232 |
| 237 | VSAGLGI | 3055 | GTCTCGGCAGGTTTAGGAATC | 3056 | 3938 | 3938 | 0 |
| 238 | NSKDVLR | 3057 | AACTCGAAAGACGTCCTCCGA | 3058 | 3937 | 3729 | 208 |
| 239 | SMQSPST | 3059 | TCTATGCAGTCGCCTAGTACG | 3060 | 3935 | 3478 | 457 |
| 240 | TLNSATT | 3061 | ACGCTTAATTCGGCGACTACT | 3062 | 3927 | 2000 | 1927 |
| 241 | SGESLRL | 3063 | TCTGGGGAGTCGCTTAGGCTT | 3064 | 3924 | 3924 | 0 |
| 242 | QVRDIKT | 3065 | CAGGTGCGTGATATTAAGACT | 3066 | 3919 | 2000 | 1919 |
| 243 | QGGNAMR | 3067 | CAGGGTGGGAATGCTATGCGT | 3068 | 3914 | 1914 | 2000 |
| 244 | MLGGGES | 3069 | ATGTTGGGTGGTGGGGAGTCG | 3070 | 3912 | 2000 | 1912 |
| 245 | PTQGTLR | 3071 | CCGACACAAGGTACACTACGC | 3072 | 3912 | 2000 | 1912 |
| 246 | VIAGLAI | 3073 | GTAATCGCCGGACTCGCCATC | 3074 | 3911 | 3697 | 214 |
| 247 | KEISVSV | 3075 | AAAGAAATCTCTGTATCTGTG | 3076 | 3905 | 3522 | 383 |
| 248 | LSANVRT | 3077 | CTGTCGGCGAATGTTCGGACT | 3078 | 3904 | 1861 | 2044 |
| 249 | KGSDNPM | 3079 | AAGGGTTCTGATAATCCTATG | 3080 | 3902 | 3427 | 475 |
| 250 | GAPSGSL | 3081 | GGAGCTCCGTCAGGATCCCTT | 3082 | 3900 | 1538 | 2362 |
| 251 | TSGNAGL | 3083 | ACAAGCGGGAACGCGGGCCTC | 3084 | 3899 | 3899 | 0 |
| 252 | REQQKLW | 3085 | AGGGAACAACAAAAATTATGG | 3086 | 3898 | 3425 | 473 |
| 253 | NVTGVVL | 3087 | AACGTAACTGGAGTTGTCCTT | 3088 | 3896 | 2913 | 983 |
| 254 | MESVTQG | 3089 | ATGGAAAGCGTAACCCAAGGA | 3090 | 3884 | 3884 | 0 |
| 255 | TTVKVSP | 3091 | ACGACTGTGAAGGTTAGTCCT | 3092 | 3876 | 3876 | 0 |
| 256 | GLPDTMA | 3093 | GGTCTGCCAGACACGATGGCC | 3094 | 3869 | 0 | 3869 |
| 257 | QSQTADA | 3095 | CAGTCTCAGACGGCTGATGCT | 3096 | 3861 | 3861 | 0 |
| 258 | TLTLSMR | 3097 | ACACTTACGCTTTCAATGAGG | 3098 | 3854 | 0 | 3854 |
| 259 | GNPGSHS | 3099 | GGGAATCCGGGTTCTCATAGT | 3100 | 3849 | 2000 | 1849 |
| 260 | SGNQPRM | 3101 | TCTGGGAATCAGCCTAGGATG | 3102 | 3830 | 2466 | 1364 |
| 261 | SHGSDLK | 3103 | TCTCATGGTTCTGATTTGAAG | 3104 | 3828 | 3516 | 312 |
| 262 | SLGEGRH | 3105 | TCGTTGGGTGAGGGTCGGCAT | 3106 | 3816 | 2000 | 1816 |
| 263 | AGISSQP | 3107 | GCTGGCATCAGCTCACAACCA | 3108 | 3812 | 3288 | 524 |
| 264 | SLGEARP | 3109 | TCTTTAGGGGAAGCGCGTCCC | 3110 | 3811 | 3390 | 421 |
| 265 | IYSDGSS | 3111 | ATTTATAGTGATGGGTCGTCT | 3112 | 3807 | 1426 | 2381 |
| 266 | RVSLAVK | 3113 | AGGGTGTCGCTGGCTGTGAAG | 3114 | 3802 | 3456 | 346 |
| 267 | HGTGNTY | 3115 | CACGGAACCGGTAACACATAC | 3116 | 3799 | 2000 | 1799 |
| 268 | TSERGSL | 3117 | ACGAGTGAGAGGGGGTCGCTG | 3118 | 3790 | 3071 | 718 |
| 269 | ASPQVSL | 3119 | GCGAGTCCGCAGGTGTCGCTT | 3120 | 3789 | 1789 | 2000 |
| 270 | VVQDPGR | 3121 | GTTGTTCAGGATCCTGGGCGT | 3122 | 3781 | 3781 | 0 |
| 271 | QGGDSGG | 3123 | CAGGGTGGTGATAGTGGGGGT | 3124 | 3781 | 2000 | 1781 |
| 272 | TADARAL | 3125 | ACAGCGGACGCGCGCGCTTTG | 3126 | 3778 | 3778 | 0 |
| 273 | TNGHSQV | 3127 | ACTAACGGACACAGCCAAGTC | 3128 | 3763 | 1456 | 2307 |
| 274 | RNQAEEM | 3129 | CGTAACCAAGCCGAAGAAATG | 3130 | 3762 | 3762 | 0 |
| 275 | VAVSSNK | 3131 | GTTGCTGTTTCTTCGAATAAG | 3132 | 3759 | 1551 | 2208 |
| 276 | KTAQVQP | 3133 | AAAACCGCTCAAGTCCAACCT | 3134 | 3750 | 3483 | 267 |
| 277 | AVGSDGV | 3135 | GCTGTGGGTTCTGATGGTGTT | 3136 | 3748 | 1748 | 2000 |
| 278 | LNSSTQR | 3137 | TTGAATTCTAGTACGCAGCGT | 3138 | 3738 | 1738 | 2000 |
| 279 | QMKSRSD | 3139 | CAGATGAAGTCTCGTTCTGAT | 3140 | 3727 | 3470 | 257 |
| 280 | SVQITGL | 3141 | TCTGTGCAGATTACTGGTTTG | 3142 | 3727 | 1338 | 2389 |
| 281 | STLQGVA | 3143 | AGTACGCTGCAGGGTGTGGCT | 3144 | 3726 | 3726 | 0 |
| 282 | KLAEGVR | 3145 | AAGCTTGCTGAGGGGGTTAGG | 3146 | 3723 | 2000 | 1723 |
| 283 | TDKQNAF | 3147 | ACAGACAAACAAAACGCCTTC | 3148 | 3720 | 1664 | 2056 |
| 284 | REIRVSV | 3149 | CGAGAAATCAGAGTATCCGTC | 3150 | 3710 | 1710 | 2000 |
| 285 | QVRDAKT | 3151 | CAGGTGCGTGATGCTAAGACT | 3152 | 3698 | 2000 | 1698 |
| 286 | VEGPTTN | 3153 | GTCGAAGGGCCTACAACGAAC | 3154 | 3694 | 0 | 3694 |
| 287 | SDRTAVV | 3155 | AGTGATCGGACGGCGGTTGTT | 3156 | 3693 | 734 | 2959 |
| 288 | QSDNHGR | 3157 | CAGTCGGATAATCATGGTAGG | 3158 | 3692 | 3163 | 529 |
| 289 | ETQGRQF | 3159 | GAGACTCAGGGTCGTCAGTTT | 3160 | 3677 | 3677 | 0 |
| 290 | PSVSTLS | 3161 | CCGTCGGTCAGCACACTGTCG | 3162 | 3676 | 3367 | 309 |
| 291 | RGQSDPA | 3163 | CGGGGGCAGTCTGATCCGGCG | 3164 | 3652 | 3652 | 0 |
| 292 | AKESGLM | 3165 | GCAAAAGAATCCGGCCTAATG | 3166 | 3652 | 0 | 3652 |
| 293 | AEGRSAM | 3167 | GCGGAGGGGAGGAGTGCTATG | 3168 | 3648 | 2000 | 1648 |
| 294 | RLNEHVA | 3169 | CGGTTGAACGAACACGTTGCC | 3170 | 3640 | 2000 | 1640 |
| 295 | TGASTFV | 3171 | ACCGGAGCCAGTACATTCGTA | 3172 | 3640 | 1690 | 1950 |
| 296 | ALIRDNV | 3173 | GCTCTGATTCGTGATAATGTT | 3174 | 3628 | 2000 | 1628 |
| 297 | PHAATPG | 3175 | CCTCATGCTGCGACTCCTGGG | 3176 | 3625 | 1625 | 2000 |
| 298 | RSAGTSS | 3177 | CGTTCGGCGGGTACTTCGTCG | 3178 | 3624 | 3624 | 0 |
| 299 | SKAEGPV | 3179 | TCGAAGGCTGAGGGTCCGGTT | 3180 | 3620 | 3620 | 0 |
| 300 | LSLRDGV | 3181 | CTCTCGCTCCGGGACGGAGTC | 3182 | 3619 | 2000 | 1619 |
| 301 | TVSEQPR | 3183 | ACGGTTTCGGAGCAGCCGCGT | 3184 | 3616 | 3616 | 0 |
| 302 | LSLGSQL | 3185 | CTGTCTCTGGGGTCGCAGCTG | 3186 | 3606 | 1380 | 2226 |
| 303 | NGTAGDR | 3187 | AACGGAACCGCAGGGGACCGC | 3188 | 3604 | 3347 | 257 |
| 304 | VQVSSVA | 3189 | GTTCAGGTTTCTTCTGTGGCT | 3190 | 3601 | 1601 | 2000 |
| 305 | SDGKTHP | 3191 | TCGGATGGTAAGACTCATCCG | 3192 | 3601 | 1190 | 2411 |
| 306 | NEPSVNT | 3193 | AACGAACCCTCGGTAAACACG | 3194 | 3599 | 3599 | 0 |
| 307 | DVADSKR | 3195 | GATGTTGCTGATTCTAAGCGT | 3196 | 3590 | 3097 | 493 |
| 308 | GVAGTYS | 3197 | GGCGTCGCAGGTACCTACAGT | 3198 | 3583 | 3583 | 0 |
| 309 | NIKDVNR | 3199 | AATATTAAGGATGTTAATAGG | 3200 | 3577 | 2000 | 1577 |
| 310 | DLVLLHR | 3201 | GATCTTGTTTTGTTGCATAGG | 3202 | 3568 | 0 | 3568 |
| 311 | SNGTVTI | 3203 | TCAAACGGGACTGTAACAATA | 3204 | 3559 | 3230 | 329 |
| 312 | LANTLSV | 3205 | CTGGCTAATACGTTGAGTGTT | 3206 | 3556 | 3556 | 0 |
| 313 | PTQVTLR | 3207 | CCTACTCAGGTGACGCTTCGG | 3208 | 3548 | 3548 | 0 |
| 314 | ILGGLAV | 3209 | ATATTGGGCGGCCTAGCCGTG | 3210 | 3529 | 1529 | 2000 |
| 315 | TAVHSAS | 3211 | ACGGCTGTGCATTCGGCTTCG | 3212 | 3526 | 3141 | 385 |
| 316 | TSLSQDR | 3213 | ACGAGTTTGTCGCAGGATAGG | 3214 | 3524 | 0 | 3524 |
| 317 | LDTTARL | 3215 | CTTGATACTACTGCTCGTCTT | 3216 | 3517 | 1517 | 2000 |
| 318 | GASDQLS | 3217 | GGTGCTTCGGATCAGCTTTCT | 3218 | 3514 | 3514 | 0 |
| 319 | EKVAPTP | 3219 | GAGAAGGTTGCTCCGACGCCT | 3220 | 3514 | 2000 | 1514 |
| 320 | AAGGQVL | 3221 | GCGGCGGGTGGGCAGGTTCTG | 3222 | 3512 | 3512 | 0 |
| 321 | PGDRSPS | 3223 | CCGGGTGATCGTTCGCCTTCT | 3224 | 3508 | 3508 | 0 |
| 322 | DALSRMA | 3225 | GACGCATTGTCACGTATGGCT | 3226 | 3498 | 0 | 3498 |
| 323 | TAGQVSK | 3227 | ACTGCGGGTCAGGTGTCTAAG | 3228 | 3495 | 0 | 3495 |
| 324 | LAKDSGG | 3229 | TTGGCTAAGGATTCTGGGGGG | 3230 | 3488 | 1163 | 2325 |
| 325 | SAGRADL | 3231 | AGTGCAGGTAGAGCCGACCTC | 3232 | 3482 | 3482 | 0 |
| 326 | TTAAIVS | 3233 | ACAACAGCTGCGATAGTATCC | 3234 | 3474 | 3117 | 357 |
| 327 | SMGQKEL | 3235 | AGCATGGGCCAAAAAGAACTA | 3236 | 3474 | 2000 | 1474 |
| 328 | PQKGGGV | 3237 | CCTCAGAAGGGTGGTGGGGTG | 3238 | 3472 | 2000 | 1472 |
| 329 | GAVSSIK | 3239 | GGGGCCGTGAGTAGTATCAAA | 3240 | 3472 | 1150 | 2322 |
| 330 | LSSGVSK | 3241 | CTCTCATCGGGTGTATCCAAA | 3242 | 3466 | 1524 | 1942 |
| 331 | SVGLTNG | 3243 | AGTGTGGGTCTGACGAATGGT | 3244 | 3465 | 780 | 2685 |
| 332 | VKQTDVA | 3245 | GTTAAGCAGACGGATGTTGCT | 3246 | 3461 | 3461 | 0 |
| 333 | EAVRLSA | 3247 | GAAGCCGTACGGCTGTCCGCA | 3248 | 3461 | 1353 | 2108 |
| 334 | GTLTSGY | 3249 | GGCACACTCACGAGCGGATAC | 3250 | 3460 | 2000 | 1460 |
| 335 | KASDTPK | 3251 | AAGGCTTCTGATACTCCTAAG | 3252 | 3457 | 3451 | 6 |
| 336 | RGGVTGE | 3253 | CGTGGTGGCGTGACAGGAGAA | 3254 | 3450 | 3450 | 0 |
| 337 | AVLAGYR | 3255 | GCAGTCCTTGCAGGCTACCGT | 3256 | 3448 | 3448 | 0 |
| 338 | GTYSTSL | 3257 | GGTACGTACAGCACCAGCCTC | 3258 | 3448 | 2000 | 1448 |
| 339 | TSLGLVV | 3259 | ACATCTCTTGGGTTGGTAGTT | 3260 | 3445 | 3445 | 0 |
| 340 | QVRDTKI | 3261 | CAGGTGCGTGATACTAAGATT | 3262 | 3445 | 3445 | 0 |
| 341 | SMGQKEL | 3263 | TCTATGGGGCAGAAGGAGCTT | 3264 | 3437 | 3247 | 190 |
| 342 | KEAPHGV | 3265 | AAAGAAGCCCCCCACGGTGTA | 3266 | 3431 | 1077 | 2354 |
| 343 | KSLSENS | 3267 | AAATCATTGTCCGAAAACAGC | 3268 | 3422 | 3152 | 270 |
| 344 | SEVSKGK | 3269 | AGTGAGGTTAGTAAGGGTAAG | 3270 | 3420 | 3087 | 333 |
| 345 | RTQTDLG | 3271 | CGGACGCAGACGGATCTTGGT | 3272 | 3414 | 3414 | 0 |
| 346 | TTLGATA | 3273 | ACGACGTTGGGTGCTACGGCT | 3274 | 3410 | 2000 | 1410 |
| 347 | ITSGTGT | 3275 | ATTACTAGTGGTACGGGTACT | 3276 | 3408 | 3290 | 118 |
| 348 | QTQTAVR | 3277 | CAGACTCAGACGGCTGTTCGT | 3278 | 3408 | 2000 | 1408 |
| 349 | LADNHGR | 3279 | TTGGCTGACAACCACGGCCGT | 3280 | 3407 | 1407 | 2000 |
| 350 | NQINAGV | 3281 | AACCAAATCAACGCCGGCGTG | 3282 | 3399 | 1399 | 2000 |
| 351 | SHGSDSR | 3283 | TCTCATGGTTCTGATTCGAGG | 3284 | 3385 | 2963 | 421 |
| 352 | SQKEVAT | 3285 | AGCCAAAAAGAAGTTGCCACC | 3286 | 3383 | 3383 | 0 |
| 353 | KEGAVYV | 3287 | AAGGAGGGGGCGGTGTATGTG | 3288 | 3380 | 1615 | 1765 |
| 354 | VSGSISK | 3289 | GTTTCGGGGAGTATTTCTAAG | 3290 | 3370 | 1230 | 2140 |
| 355 | VSPGKLH | 3291 | GTCAGTCCTGGCAAACTCCAC | 3292 | 3367 | 2000 | 1367 |
| 356 | GETNTNI | 3293 | GGGGAAACAAACACCAACATC | 3294 | 3365 | 3365 | 0 |
| 357 | TYGSGPT | 3295 | ACTTATGGTTCTGGGCCTACT | 3296 | 3362 | 1362 | 2000 |
| 358 | SSGQLGV | 3297 | TCCTCCGGCCAATTAGGCGTT | 3298 | 3360 | 2800 | 560 |
| 359 | WAISDGY | 3299 | TGGGCTATATCAGACGGATAC | 3300 | 3360 | 1931 | 1429 |
| 360 | HAEGARS | 3301 | CATGCGGAGGGTGCTCGTAGT | 3302 | 3356 | 3356 | 0 |
| 361 | SGEALRL | 3303 | TCTGGGGAGGCGCTTAGGCTT | 3304 | 3355 | 0 | 3355 |
| 362 | SMGIGAV | 3305 | TCTATGGGGATTGGTGCGGTT | 3306 | 3347 | 3347 | 0 |
| 363 | MVKQQLT | 3307 | ATGGTCAAACAACAACTCACC | 3308 | 3347 | 2974 | 373 |
| 364 | LTSGQAV | 3309 | TTGACATCTGGCCAAGCAGTC | 3310 | 3347 | 1067 | 2280 |
| 365 | LSLTNGV | 3311 | TTGAGCCTTACAAACGGTGTG | 3312 | 3346 | 3346 | 0 |
| 366 | SGANLSI | 3313 | TCAGGAGCAAACCTCAGCATC | 3314 | 3345 | 2000 | 1345 |
| 367 | SHLNTTP | 3315 | TCGCATTTGAATACTACTCCT | 3316 | 3342 | 1999 | 1343 |
| 368 | VMSGTSH | 3317 | GTGATGTCAGGCACGTCTCAC | 3318 | 3339 | 1339 | 2000 |
| 369 | REQQKIW | 3319 | CGTGAGCAGCAGAAGATTTGG | 3320 | 3332 | 2296 | 1037 |
| 370 | DGQRLGA | 3321 | GACGGCCAAAGATTAGGTGCG | 3322 | 3331 | 1848 | 1483 |
| 371 | VLASPGH | 3323 | GTACTAGCGAGTCCGGGACAC | 3324 | 3328 | 0 | 3328 |
| 372 | NSKDGHR | 3325 | AACTCAAAAGACGGACACCGT | 3326 | 3326 | 3326 | 0 |
| 373 | KNGGHVL | 3327 | AAAAACGGCGGCCACGTGTTG | 3328 | 3326 | 3162 | 164 |
| 374 | ALSGLAR | 3329 | GCCCTCTCAGGCTTAGCACGT | 3330 | 3323 | 2000 | 1323 |
| 375 | TSGNAGL | 3331 | ACTTCTGGTAATGCTGGGCTT | 3332 | 3322 | 2722 | 600 |
| 376 | AGSDYTV | 3333 | GCTGGTAGTGATTATACTGTG | 3334 | 3321 | 1325 | 1996 |
| 377 | TQIETRR | 3335 | ACGCAGATTGAGACGAGGCGG | 3336 | 3317 | 3317 | 0 |
| 378 | LISSTQR | 3337 | TTGATTTCTAGTACGCAGCGT | 3338 | 3313 | 2000 | 1313 |
| 379 | KIAEGIR | 3339 | AAGATTGCTGAGGGGATTAGG | 3340 | 3310 | 0 | 3310 |
| 380 | PSPGSQL | 3341 | CCGTCTCCGGGGTCGCAGCTG | 3342 | 3307 | 0 | 3307 |
| 381 | RVESADL | 3343 | CGCGTAGAATCCGCAGACCTC | 3344 | 3301 | 2000 | 1301 |
| 382 | KGSDSPK | 3345 | AAGGGTTCTGATTCTCCTAAG | 3346 | 3293 | 2985 | 308 |
| 383 | TVGHADK | 3347 | ACGGTTGGGCATGCTGATAAG | 3348 | 3290 | 959 | 2332 |
| 384 | VDRSGIP | 3349 | GTAGACCGTTCTGGAATACCA | 3350 | 3289 | 0 | 3289 |
| 385 | QVRDTKT | 3351 | CAGGTGCGTGATACTAAGACT | 3352 | 3288 | 1288 | 2000 |
| 386 | RNNVETT | 3353 | CGTAACAACGTGGAAACAACA | 3354 | 3281 | 3281 | 0 |
| 387 | AVISMTP | 3355 | GCCGTAATATCTATGACCCCG | 3356 | 3273 | 1273 | 2000 |
| 388 | ATEKAVR | 3357 | GCGACGGAGAAGGCTGTGAGG | 3358 | 3269 | 2811 | 458 |
| 389 | SADVTAR | 3359 | AGTGCTGATGTGACGGCGAGG | 3360 | 3267 | 1267 | 2000 |
| 390 | GTGGAVR | 3361 | GGAACCGGCGGGGCAGTGCGC | 3362 | 3266 | 2000 | 1266 |
| 391 | RTMGDST | 3363 | CGTACAATGGGCGACTCAACG | 3364 | 3257 | 3257 | 0 |
| 392 | LGDSAKP | 3365 | CTAGGCGACTCAGCCAAACCT | 3366 | 3257 | 2000 | 1257 |
| 393 | VTQGTSL | 3367 | GTTACGCAGGGGACTTCTCTG | 3368 | 3252 | 1435 | 1817 |
| 394 | IAVGLTV | 3369 | ATTGCGGTGGGGCTGACTGTT | 3370 | 3251 | 0 | 3251 |
| 395 | FTPGTAT | 3371 | TTTACTCCTGGTACGGCTACT | 3372 | 3235 | 2000 | 1235 |
| 396 | LGDSASP | 3373 | CTTGGGGATTCTGCTTCGCCG | 3374 | 3224 | 1224 | 2000 |
| 397 | VGVPTTN | 3375 | GTAGGAGTACCCACCACGAAC | 3376 | 3217 | 1217 | 2000 |
| 398 | LLADKRA | 3377 | CTTCTTGCTGATAAGCGTGCG | 3378 | 3215 | 1004 | 2211 |
| 399 | TDGRGDR | 3379 | ACGGATGGTCGGGGTGATAGG | 3380 | 3212 | 779 | 2433 |
| 400 | NLIKPFL | 3381 | AATTTGATTAAGCCTTTTCTT | 3382 | 3209 | 2000 | 1209 |
| 401 | EIALTVH | 3383 | GAAATAGCACTGACAGTACAC | 3384 | 3207 | 0 | 3207 |
| 402 | NSKDVQR | 3385 | AATAGTAAGGATGTTCAGAGG | 3386 | 3206 | 2470 | 736 |
| 403 | DLVLLHR | 3387 | GACTTAGTCCTCCTTCACCGA | 3388 | 3199 | 0 | 3199 |
| 404 | MSVQDRG | 3389 | ATGTCCGTTCAAGACCGAGGC | 3390 | 3199 | 0 | 3199 |
| 405 | DRSTTVP | 3391 | GATCGTAGTACGACGGTTCCT | 3392 | 3197 | 3197 | 0 |
| 406 | LSRGSQL | 3393 | TTAAGTCGCGGTTCACAACTT | 3394 | 3197 | 2950 | 247 |
| 407 | THLSSTR | 3395 | ACGCATCTGAGTAGTACTCGT | 3396 | 3196 | 3196 | 0 |
| 408 | MNGGHAL | 3397 | ATGAACGGAGGGCACGCATTG | 3398 | 3193 | 3193 | 0 |
| 409 | SPSAFPI | 3399 | TCGCCTAGTGCATTCCCAATC | 3400 | 3192 | 0 | 3192 |
| 410 | DYGSTGR | 3401 | GATTATGGTTCTACGGGGCGG | 3402 | 3185 | 3185 | 0 |
| 411 | QVAQQGA | 3403 | CAAGTCGCACAACAAGGCGCT | 3404 | 3184 | 2792 | 392 |
| 412 | IGSGVLA | 3405 | ATTGGTAGTGGTGTTCTTGCT | 3406 | 3184 | 0 | 3184 |
| 413 | VGHGGVD | 3407 | GTGGGTCATGGTGGTGTGGAT | 3408 | 3176 | 1176 | 2000 |
| 414 | AVGSTVK | 3409 | GCGGTTGGGTCGACGGTGAAG | 3410 | 3170 | 3170 | 0 |
| 415 | SGEQLRI | 3411 | TCTGGGGAGCAGCTTAGGATT | 3412 | 3168 | 2000 | 1168 |
| 416 | RNNVDST | 3413 | CGGAATAATGTTGATTCTACG | 3414 | 3164 | 3164 | 0 |
| 417 | AVGTAIG | 3415 | GCGGTAGGCACGGCAATCGGC | 3416 | 3160 | 2000 | 1160 |
| 418 | VAGLGGL | 3417 | GTTGCGGGTTTGGGGGGGCTT | 3418 | 3154 | 0 | 3154 |
| 419 | RTSAGVV | 3419 | AGGACGAGTGCGGGTGTTGTG | 3420 | 3151 | 3151 | 0 |
| 420 | FTSGTGN | 3421 | TTCACGAGCGGAACAGGCAAC | 3422 | 3149 | 3149 | 0 |
| 421 | QREATRI | 3423 | CAGCGTGAGGCGACGCGGATT | 3424 | 3140 | 2000 | 1140 |
| 422 | LNNPVQV | 3425 | CTGAATAATCCTGTGCAGGTT | 3426 | 3137 | 0 | 3137 |
| 423 | LISTTLR | 3427 | TTGATTTCTACTACGCTGCGT | 3428 | 3136 | 0 | 3136 |
| 424 | GGTVSGH | 3429 | GGAGGGACTGTATCTGGACAC | 3430 | 3134 | 2000 | 1134 |
| 425 | AGTLYAR | 3431 | GCGGGGACTCTGTATGCTCGT | 3432 | 3134 | 2000 | 1134 |
| 426 | GQSSNLH | 3433 | GGACAAAGTAGCAACTTGCAC | 3434 | 3132 | 0 | 3132 |
| 427 | DVSGSVI | 3435 | GACGTAAGCGGCTCCGTAATC | 3436 | 3130 | 2000 | 1130 |
| 428 | KLAEGVR | 3437 | AAATTGGCAGAAGGAGTCAGA | 3438 | 3123 | 2000 | 1123 |
| 429 | SGLQVSI | 3439 | TCAGGATTGCAAGTGTCGATA | 3440 | 3120 | 3120 | 0 |
| 430 | QTGAIVV | 3441 | CAAACAGGAGCAATCGTTGTC | 3442 | 3113 | 1671 | 1442 |
| 431 | LEANVSH | 3443 | CTGGAAGCTAACGTGAGTCAC | 3444 | 3112 | 2000 | 1112 |
| 432 | ALSGLSK | 3445 | GCTCTTTCTGGTCTTTCTAAG | 3446 | 3108 | 0 | 3108 |
| 433 | MGKQTTL | 3447 | ATGGGGAAGCAGACTACGCTG | 3448 | 3107 | 2664 | 443 |
| 434 | HHSQYGA | 3449 | CACCACTCGCAATACGGCGCT | 3450 | 3106 | 0 | 3106 |
| 435 | TGLQGSI | 3451 | ACCGGCCTTCAAGGATCTATA | 3452 | 3101 | 3101 | 0 |
| 436 | LISGEKT | 3453 | TTGATTTCTGGTGAGAAGACG | 3454 | 3101 | 2000 | 1101 |
| 437 | RSASGNE | 3455 | CGGAGTGCTAGTGGTAATGAG | 3456 | 3099 | 3099 | 0 |
| 438 | SEKSVPL | 3457 | TCCGAAAAAAGCGTACCACTG | 3458 | 3097 | 2000 | 1097 |
| 439 | VLDSRSP | 3459 | GTTCTGGATAGTAGGAGTCCG | 3460 | 3096 | 2423 | 673 |
| 440 | QGGNSGR | 3461 | CAAGGAGGCAACTCAGGTAGG | 3462 | 3095 | 1095 | 2000 |
| 441 | SAVASGK | 3463 | TCGGCGGTGGCGTCGGGTAAG | 3464 | 3094 | 2000 | 1094 |
| 442 | AQGPQTG | 3465 | GCGCAGGGTCCGCAGACTGGT | 3466 | 3092 | 1092 | 2000 |
| 443 | MNVGNVL | 3467 | ATGAACGTTGGGAACGTGCTC | 3468 | 3092 | 0 | 3092 |
| 444 | VLGGTGK | 3469 | GTCCTCGGAGGTACCGGTAAA | 3470 | 3089 | 2941 | 148 |
| 445 | KSHSEIS | 3471 | AAATCCCACAGTGAAATCAGC | 3472 | 3088 | 0 | 3088 |
| 446 | SDSRVSY | 3473 | AGTGACTCCCGAGTATCGTAC | 3474 | 3087 | 0 | 3087 |
| 447 | YTAGSMA | 3475 | TATACGGCGGGGTCTATGGCG | 3476 | 3086 | 1086 | 2000 |
| 448 | TRFDGSG | 3477 | ACGCGTTTTGATGGTTCGGGT | 3478 | 3084 | 1084 | 2000 |
| 449 | QREAERI | 3479 | CAGCGTGAGGCGGAGCGGATT | 3480 | 3077 | 2000 | 1077 |
| 450 | KNPAVDP | 3481 | AAAAACCCCGCAGTCGACCCG | 3482 | 3076 | 3076 | 0 |
| 451 | FSSETLT | 3483 | TTCAGCTCCGAAACCTTGACC | 3484 | 3072 | 1594 | 1478 |
| 452 | YGNSGVI | 3485 | TACGGCAACTCGGGGGTCATA | 3486 | 3062 | 694 | 2369 |
| 453 | KNPGADP | 3487 | AAAAACCCTGGTGCCGACCCC | 3488 | 3058 | 3058 | 0 |
| 454 | LAIAGTM | 3489 | CTTGCTATTGCGGGGACTATG | 3490 | 3057 | 0 | 3057 |
| 455 | SNLGNTS | 3491 | TCGAATCTGGGTAATACTAGT | 3492 | 3052 | 1052 | 2000 |
| 456 | PIQLGQA | 3493 | CCGATTCAGTTGGGTCAGGCT | 3494 | 3051 | 2000 | 1051 |
| 457 | KTETGYE | 3495 | AAGACTGAGACTGGTTATGAG | 3496 | 3051 | 2000 | 1051 |
| 458 | VTKVSHV | 3497 | GTCACAAAAGTAAGTCACGTC | 3498 | 3046 | 2000 | 1046 |
| 459 | AGGGVPR | 3499 | GCAGGAGGCGGCGTCCCACGT | 3500 | 3043 | 1875 | 1168 |
| 460 | ADKGGVA | 3501 | GCTGATAAGGGGGGTGTGGCT | 3502 | 3042 | 2903 | 139 |
| 461 | SEGISRY | 3503 | TCAGAAGGCATATCTCGGTAC | 3504 | 3040 | 0 | 3040 |
| 462 | LDHGGVD | 3505 | CTCGACCACGGAGGAGTAGAC | 3506 | 3038 | 2000 | 1038 |
| 463 | NEQSVKT | 3507 | AACGAACAAAGCGTTAAAACC | 3508 | 3037 | 2000 | 1037 |
| 464 | SARDMTR | 3509 | AGCGCCCGCGACATGACTCGT | 3510 | 3034 | 2000 | 1034 |
| 465 | TSVGMQV | 3511 | ACGTCGGTTGGGATGCAGGTT | 3512 | 3033 | 738 | 2295 |
| 466 | VQAGKEL | 3513 | GTGCAGGCTGGTAAGGAGTTG | 3514 | 3033 | 0 | 3033 |
| 467 | NASAGDR | 3515 | AATGCGAGTGCGGGTGATCGT | 3516 | 3028 | 3028 | 0 |
| 468 | AAGVILK | 3517 | GCGGCGGGTGTTATTCTGAAG | 3518 | 3028 | 3028 | 0 |
| 469 | SGAEGGR | 3519 | TCTGGTGCTGAGGGTGGTCGG | 3520 | 3028 | 3028 | 0 |
| 470 | NRQEHSN | 3521 | AACCGCCAAGAACACAGCAAC | 3522 | 3028 | 2000 | 1028 |
| 471 | AADGSVR | 3523 | GCCGCAGACGGAAGTGTTAGG | 3524 | 3028 | 2000 | 1028 |
| 472 | SGANLSM | 3525 | TCTGGTGCGAATTTGTCTATG | 3526 | 3027 | 1027 | 2000 |
| 473 | HSSGWTS | 3527 | CACTCGAGTGGATGGACCAGC | 3528 | 3022 | 0 | 3022 |
| 474 | NLGVVQP | 3529 | AATTTGGGTGTGGTTCAGCCG | 3530 | 3022 | 0 | 3022 |
| 475 | HSTGAEK | 3531 | CATTCGACGGGTGCGGAGAAG | 3532 | 3020 | 3020 | 0 |
| 476 | NLSISER | 3533 | AATTTGTCGATTTCTGAGCGG | 3534 | 3018 | 0 | 3018 |
| 477 | TNLADTA | 3535 | ACTAATCTGGCTGATACTGCG | 3536 | 3013 | 2000 | 1013 |
| 478 | RSSGTSA | 3537 | AGATCATCCGGGACCTCAGCA | 3538 | 3011 | 3011 | 0 |
| 479 | KNGGHVL | 3539 | AAGAATGGGGGTCATGTTCTG | 3540 | 3010 | 788 | 2222 |
| 480 | GSSGGHF | 3541 | GGAAGCTCGGGTGGACACTTC | 3542 | 3005 | 2000 | 1005 |
| 481 | ISHSESV | 3543 | ATTAGTCATTCGGAGAGTGTG | 3544 | 3005 | 0 | 3005 |
| 482 | VTGVSRV | 3545 | GTTACGGGTGTGAGTCGTGTG | 3546 | 3003 | 2000 | 1003 |
| 483 | QGGNSGA | 3547 | CAGGGTGGTAATAGTGGGGCT | 3548 | 3003 | 0 | 3003 |
| 484 | AMPTSGH | 3549 | GCGATGCCGACGAGTGGGCAT | 3550 | 3000 | 0 | 3000 |
| 485 | TLTNGMP | 3551 | ACCTTGACCAACGGTATGCCA | 3552 | 2999 | 0 | 2999 |
| 486 | SHGTDSK | 3553 | TCCCACGGAACGGACAGTAAA | 3554 | 2998 | 2998 | 0 |
| 487 | WSDRESR | 3555 | TGGTCTGACCGCGAATCTAGG | 3556 | 2997 | 1759 | 1238 |
| 488 | KEIRVSV | 3557 | AAAGAAATAAGGGTCTCCGTG | 3558 | 2992 | 2992 | 0 |
| 489 | FAGVTQA | 3559 | TTTGCGGGGGTTACGCAGGCG | 3560 | 2989 | 0 | 2989 |
| 490 | DSHVSGV | 3561 | GACTCTCACGTATCCGGAGTG | 3562 | 2988 | 1654 | 1334 |
| 491 | LLKESTP | 3563 | CTCCTTAAAGAAAGTACACCT | 3564 | 2984 | 0 | 2984 |
| 492 | ADREVRY | 3565 | GCGGATCGGGAGGTGCGTTAT | 3566 | 2982 | 0 | 2982 |
| 493 | MNGGHGL | 3567 | ATGAATGGGGGTCATGGTCTG | 3568 | 2980 | 2000 | 980 |
| 494 | SLRDVEG | 3569 | TCGCTGCGTGATGTGGAGGGT | 3570 | 2979 | 2000 | 979 |
| 495 | SKSGVVA | 3571 | AGTAAGTCTGGTGTGGTGGCG | 3572 | 2979 | 1575 | 1404 |
| 496 | RNEGSVP | 3573 | CGAAACGAAGGCTCGGTCCCT | 3574 | 2978 | 2000 | 978 |
| 497 | NLQGNAL | 3575 | AACTTACAAGGCAACGCGCTA | 3576 | 2977 | 2000 | 977 |
| 498 | YSTTAGM | 3577 | TATTCGACTACGGCTGGTATG | 3578 | 2973 | 0 | 2973 |
| 499 | AADGSVR | 3579 | GCGGCGGATGGTTCTGTGCGG | 3580 | 2972 | 2000 | 972 |
| 500 | VGNMLSV | 3581 | GTCGGGAACATGCTATCTGTG | 3582 | 2970 | 2000 | 970 |
| 501 | KEYITAV | 3583 | AAGGAGTATATTACGGCTGTG | 3584 | 2969 | 2969 | 0 |
| 502 | ANAGMSR | 3585 | GCGAATGCTGGGATGTCTAGG | 3586 | 2969 | 2000 | 969 |
| 503 | HTVEGAL | 3587 | CATACGGTTGAGGGGGCGCTG | 3588 | 2967 | 2000 | 967 |
| 504 | NHQSLVN | 3589 | AACCACCAATCGCTCGTTAAC | 3590 | 2963 | 2000 | 963 |
| 505 | VSGTLLA | 3591 | GTATCCGGCACGTTACTGGCA | 3592 | 2963 | 0 | 2963 |
| 506 | QSRPDAL | 3593 | CAGAGTCGTCCGGATGCTCTT | 3594 | 2958 | 2000 | 958 |
| 507 | AGVVNGL | 3595 | GCGGGGGTGGTGAATGGTTTG | 3596 | 2956 | 2000 | 956 |
| 508 | RGGETSE | 3597 | CGGGGGGGTGAGACGTCTGAG | 3598 | 2953 | 2953 | 0 |
| 509 | LSLTVGV | 3599 | CTGAGTTTGACTGTTGGGGTT | 3600 | 2952 | 2000 | 952 |
| 510 | HISSLAM | 3601 | CACATATCCTCCCTTGCCATG | 3602 | 2951 | 0 | 2951 |
| 511 | AFSGGET | 3603 | GCCTTCAGCGGTGGTGAAACG | 3604 | 2948 | 1766 | 1182 |
| 512 | LRGTENQ | 3605 | TTGCGTGGGACGGAGAATCAG | 3606 | 2948 | 4 | 2944 |
| 513 | QSQTAVD | 3607 | CAATCACAAACAGCAGTCGAC | 3608 | 2946 | 0 | 2946 |
| 514 | ASSATLL | 3609 | GCGTCTAGTGCTACTTTGTTG | 3610 | 2945 | 2000 | 945 |
| 515 | GQALVSS | 3611 | GGTCAAGCTTTAGTGTCGAGT | 3612 | 2945 | 0 | 2945 |
| 516 | TAVHSTS | 3613 | ACGGCTGTGCATTCGACTTCG | 3614 | 2943 | 2000 | 943 |
| 517 | KNPGLDH | 3615 | AAAAACCCAGGACTAGACCAC | 3616 | 2940 | 2729 | 211 |
| 518 | KSGLLID | 3617 | AAAAGCGGCCTTCTTATAGAC | 3618 | 2937 | 2937 | 0 |
| 519 | SGVTPLR | 3619 | TCGGGAGTAACTCCACTCCGT | 3620 | 2931 | 0 | 2931 |
| 520 | REEQKVW | 3621 | AGAGAAGAACAAAAAGTCTGG | 3622 | 2926 | 2000 | 926 |
| 521 | LSQGSQM | 3623 | CTTAGTCAAGGATCCCAAATG | 3624 | 2926 | 2000 | 926 |
| 522 | LSLTATS | 3625 | CTGTCTCTGACGGCTACGTCT | 3626 | 2926 | 1867 | 1059 |
| 523 | KGSDTPK | 3627 | AAGGGTTCTGATACTCCTAAG | 3628 | 2926 | 0 | 2926 |
| 524 | SKPENAL | 3629 | TCGAAACCCGAAAACGCACTA | 3630 | 2925 | 2000 | 925 |
| 525 | GGTNSAH | 3631 | GGGGGTACGAATAGTGCTCAT | 3632 | 2924 | 2000 | 924 |
| 526 | FSTDTLS | 3633 | TTCAGCACCGACACCTTATCG | 3634 | 2924 | 0 | 2924 |
| 527 | SVDVTAK | 3635 | AGTGTTGATGTGACGGCGAAG | 3636 | 2917 | 917 | 2000 |
| 528 | VAQGSVV | 3637 | GTGGCTCAGGGGTCGGTTGTT | 3638 | 2916 | 2000 | 916 |
| 529 | TSGSGTS | 3639 | ACTTCTGGTTCTGGTACGTCG | 3640 | 2916 | 0 | 2916 |
| 530 | KEVRVSV | 3641 | AAGGAGGTTCGTGTGTCGGTT | 3642 | 2909 | 2909 | 0 |
| 531 | RVDSVQL | 3643 | AGAGTTGACTCAGTTCAACTG | 3644 | 2909 | 2000 | 909 |
| 532 | TGVQTAV | 3645 | ACTGGAGTCCAAACCGCCGTC | 3646 | 2908 | 2908 | 0 |
| 533 | AADSTER | 3647 | GCGGCGGATAGTACTGAGCGG | 3648 | 2908 | 2000 | 908 |
| 534 | GEAGKYS | 3649 | GGGGAGGCTGGGAAGTATTCT | 3650 | 2906 | 2906 | 0 |
| 535 | AGGGSPR | 3651 | GCCGGAGGCGGATCGCCTCGT | 3652 | 2903 | 2000 | 903 |
| 536 | GEAGTNS | 3653 | GGTGAAGCCGGCACAAACTCG | 3654 | 2902 | 2000 | 902 |
| 537 | RVDSSQI | 3655 | AGGGTGGATTCGTCGCAGATT | 3656 | 2902 | 0 | 2902 |
| 538 | YTAGSMA | 3657 | TACACTGCTGGCAGCATGGCC | 3658 | 2901 | 2000 | 901 |
| 539 | MLGAGVS | 3659 | ATGTTGGGTGCTGGGGTGTCG | 3660 | 2901 | 1879 | 1022 |
| 540 | QADNNGR | 3661 | CAGGCGGATAATAATGGTAGG | 3662 | 2900 | 2000 | 900 |
| 541 | TLHDKVL | 3663 | ACCTTGCACGACAAAGTCTTA | 3664 | 2899 | 2000 | 899 |
| 542 | VTKTLPQ | 3665 | GTGACCAAAACTTTGCCGCAA | 3666 | 2899 | 0 | 2899 |
| 543 | QSLTDRV | 3667 | CAGAGTTTGACTGATCGGGTT | 3668 | 2897 | 1290 | 1607 |
| 544 | ANRNESD | 3669 | GCTAATCGTAATGAGAGTGAT | 3670 | 2892 | 0 | 2892 |
| 545 | TSHDTLV | 3671 | ACGTCGCATGATACGTTGGTT | 3672 | 2886 | 2886 | 0 |
| 546 | SEGLTRY | 3673 | TCTGAAGGCCTCACCAGGTAC | 3674 | 2884 | 2884 | 0 |
| 547 | SHGADSK | 3675 | TCACACGGGGCCGACAGCAAA | 3676 | 2879 | 2615 | 263 |
| 548 | VLASTGH | 3677 | GTCTTGGCGAGCACCGGGCAC | 3678 | 2878 | 2000 | 878 |
| 549 | NHLSDRL | 3679 | AATCATCTTAGTGATCGTTTG | 3680 | 2874 | 2874 | 0 |
| 550 | MGRTDGL | 3681 | ATGGGAAGGACGGACGGATTA | 3682 | 2872 | 927 | 1945 |
| 551 | VSTERGT | 3683 | GTGAGTACTGAGCGGGGGACT | 3684 | 2871 | 2432 | 439 |
| 552 | SGHKAGV | 3685 | AGTGGTCACAAAGCAGGGGTG | 3686 | 2867 | 2000 | 867 |
| 553 | TSAEYNL | 3687 | ACGAGTGCGGAGTATAATTTG | 3688 | 2864 | 2000 | 864 |
| 554 | RSSETVA | 3689 | CGTTCATCTGAAACCGTGGCA | 3690 | 2864 | 2000 | 864 |
| 555 | NALSVKT | 3691 | AATGCGCTGTCTGTGAAGACT | 3692 | 2860 | 2860 | 0 |
| 556 | KTEQVQP | 3693 | AAGACGGAGCAGGTGCAGCCG | 3694 | 2860 | 2000 | 860 |
| 557 | RTLHDDT | 3695 | AGAACACTACACGACGACACG | 3696 | 2859 | 2000 | 859 |
| 558 | QSVSYLK | 3697 | CAGAGTGTGTCGTATCTGAAG | 3698 | 2859 | 2000 | 859 |
| 559 | ASGSAVA | 3699 | GCTTCTGGGTCGGCGGTGGCT | 3700 | 2857 | 2000 | 857 |
| 560 | MVTQQLK | 3701 | ATGGTGACGCAGCAGTTGAAG | 3702 | 2856 | 0 | 2856 |
| 561 | KNSGVDP | 3703 | AAAAACTCTGGCGTCGACCCA | 3704 | 2854 | 2854 | 0 |
| 562 | GGPAEGR | 3705 | GGAGGGCCAGCCGAAGGAAGG | 3706 | 2854 | 2000 | 854 |
| 563 | VKTSDRT | 3707 | GTTAAGACGTCGGATAGGACG | 3708 | 2853 | 2000 | 853 |
| 564 | NGVTLQV | 3709 | AACGGGGTAACCCTACAAGTA | 3710 | 2853 | 853 | 2000 |
| 565 | LSVSQSA | 3711 | CTGAGTGTTTCTCAGTCGGCG | 3712 | 2853 | 0 | 2853 |
| 566 | TRLQEGT | 3713 | ACACGTCTCCAAGAAGGCACC | 3714 | 2848 | 2000 | 848 |
| 567 | LSRGEEI | 3715 | CTTTCGAGGGGTGAGGAGATT | 3716 | 2847 | 0 | 2847 |
| 568 | SLGNSDH | 3717 | TCGTTGGGGAATTCGGATCAT | 3718 | 2843 | 2000 | 843 |
| 569 | LAGVAQA | 3719 | CTAGCTGGCGTGGCTCAAGCT | 3720 | 2839 | 2839 | 0 |
| 570 | NGQTGKH | 3721 | AATGGGCAGACGGGGAAGCAT | 3722 | 2838 | 4 | 2834 |
| 571 | VVTLGRQ | 3723 | GTGGTTACTCTGGGTCGTCAG | 3724 | 2836 | 2000 | 836 |
| 572 | LNADTDR | 3725 | CTAAACGCAGACACTGACCGG | 3726 | 2833 | 2000 | 833 |
| 573 | ASRLPQT | 3727 | GCGTCTCGGCTTCCTCAGACT | 3728 | 2831 | 0 | 2831 |
| 574 | LTPGSQL | 3729 | CTGACTCCGGGGTCGCAGCTG | 3730 | 2830 | 1828 | 1002 |
| 575 | KSSDTPM | 3731 | AAGAGTTCTGATACTCCTATG | 3732 | 2829 | 2000 | 829 |
| 576 | QIQSRSD | 3733 | CAGATTCAGTCTCGTTCTGAT | 3734 | 2828 | 2000 | 828 |
| 577 | NEIRVSV | 3735 | AATGAGATTCGTGTGTCGGTT | 3736 | 2828 | 0 | 2828 |
| 578 | LQSGVLT | 3737 | CTTCAGTCGGGTGTTCTGACT | 3738 | 2828 | 0 | 2828 |
| 579 | LSANVRN | 3739 | CTATCTGCCAACGTACGTAAC | 3740 | 2826 | 2826 | 0 |
| 580 | ASVSSPH | 3741 | GCATCGGTCAGCTCCCCACAC | 3742 | 2826 | 2000 | 826 |
| 581 | GGTINGH | 3743 | GGGGGTACGATTAATGGTCAT | 3744 | 2826 | 1789 | 1037 |
| 582 | SLAGGTP | 3745 | AGCTTAGCAGGCGGCACGCCG | 3746 | 2822 | 2000 | 822 |
| 583 | IGASVTL | 3747 | ATTGGGGCTAGTGTTACGCTT | 3748 | 2821 | 2000 | 821 |
| 584 | GYGSGEA | 3749 | GGTTATGGGTCGGGGGAGGCT | 3750 | 2820 | 2000 | 820 |
| 585 | MQKEGSP | 3751 | ATGCAGAAGGAGGGGTCGCCG | 3752 | 2817 | 2000 | 817 |
| 586 | LSANLRT | 3753 | TTATCTGCAAACCTCAGAACG | 3754 | 2814 | 2000 | 814 |
| 587 | LSANVRT | 3755 | CTCTCTGCAAACGTACGTACA | 3756 | 2813 | 853 | 1960 |
| 588 | ASLLPQP | 3757 | GCGTCTCTGCTTCCTCAGCCT | 3758 | 2813 | 0 | 2813 |
| 589 | GTGEIGM | 3759 | GGGACTGGTGAGATTGGTATG | 3760 | 2812 | 2000 | 812 |
| 590 | LGHKPGV | 3761 | CTAGGTCACAAACCAGGGGTG | 3762 | 2811 | 2000 | 811 |
| 591 | LNLTDGV | 3763 | CTGAATTTGACTGATGGGGTT | 3764 | 2810 | 2810 | 0 |
| 592 | SEVSKGM | 3765 | AGTGAGGTTAGTAAGGGTATG | 3766 | 2810 | 2000 | 810 |
| 593 | QSLTHGV | 3767 | CAGAGTTTGACTCATGGGGTT | 3768 | 2805 | 2003 | 802 |
| 594 | VVQVPAR | 3769 | GTTGTTCAGGTTCCTGCGCGT | 3770 | 2805 | 805 | 2000 |
| 595 | MNGGHAL | 3771 | ATGAATGGGGGTCATGCTCTG | 3772 | 2804 | 2000 | 804 |
| 596 | GTLTLAY | 3773 | GGAACTCTCACGCTGGCCTAC | 3774 | 2804 | 0 | 2804 |
| 597 | GAATSQI | 3775 | GGGGCAGCAACAAGCCAAATC | 3776 | 2803 | 2000 | 803 |
| 598 | PTQGSLR | 3777 | CCGACACAAGGATCTCTACGT | 3778 | 2803 | 2000 | 803 |
| 599 | VAGSSIL | 3779 | GTCGCCGGGAGTAGCATATTG | 3780 | 2803 | 2000 | 803 |
| 600 | MLGGGMS | 3781 | ATGTTGGGTGGTGGGATGTCG | 3782 | 2802 | 2000 | 802 |
| 601 | GVAGTFS | 3783 | GGGGTGGCTGGGACGTTTTCT | 3784 | 2801 | 2000 | 801 |
| 602 | TIGHSQV | 3785 | ACCATCGGACACTCACAAGTC | 3786 | 2799 | 2000 | 799 |
| 603 | QSQKDVG | 3787 | CAATCGCAAAAAGACGTAGGA | 3788 | 2798 | 1847 | 951 |
| 604 | GGTNSGH | 3789 | GGGGGTACGAATAGTGGTCAT | 3790 | 2797 | 2000 | 797 |
| 605 | GGVSSTK | 3791 | GGTGGTGTTTCTTCGACTAAG | 3792 | 2797 | 192 | 2605 |
| 606 | QVRDNNT | 3793 | CAGGTGCGTGATAATAATACT | 3794 | 2795 | 2000 | 795 |
| 607 | AGGGVPR | 3795 | GCGGGGGGTGGGGTTCCGAGG | 3796 | 2793 | 2500 | 293 |
| 608 | ASVSSPP | 3797 | GCGTCGGTAAGTAGTCCCCCG | 3798 | 2793 | 2000 | 793 |
| 609 | MNGSHVL | 3799 | ATGAATGGGAGTCATGTTCTG | 3800 | 2792 | 2000 | 792 |
| 610 | VQHSQDN | 3801 | GTGCAGCATTCGCAGGATAAT | 3802 | 2792 | 2000 | 792 |
| 611 | KGASDTL | 3803 | AAAGGCGCGTCTGACACCCTC | 3804 | 2788 | 788 | 2000 |
| 612 | SMATGVK | 3805 | TCGATGGCGACGGGTGTTAAG | 3806 | 2788 | 0 | 2788 |
| 613 | SVGSGLL | 3807 | TCGGTGGGGAGCGGTTTGCTC | 3808 | 2787 | 2000 | 787 |
| 614 | GAGSGVA | 3809 | GGTGCTGGGTCGGGGGTGGCT | 3810 | 2786 | 786 | 2000 |
| 615 | MGSGERL | 3811 | ATGGGGTCTGGGGAGCGGTTG | 3812 | 2785 | 2000 | 785 |
| 616 | TGNDVRR | 3813 | ACCGGCAACGACGTAAGACGC | 3814 | 2785 | 1797 | 988 |
| 617 | PNSGKDY | 3815 | CCCAACTCAGGCAAAGACTAC | 3816 | 2783 | 1759 | 1024 |
| 618 | LNSSTLR | 3817 | CTCAACAGTTCTACACTCAGG | 3818 | 2779 | 0 | 2779 |
| 619 | LGHKAGH | 3819 | TTGGGGCATAAGGCTGGTCAT | 3820 | 2776 | 2776 | 0 |
| 620 | LADSKDR | 3821 | TTGGCTGATAGTAAGGATCGG | 3822 | 2774 | 0 | 2774 |
| 621 | LENQSLG | 3823 | CTGGAGAATCAGAGTCTTGGT | 3824 | 2771 | 0 | 2771 |
| 622 | QYVVSGV | 3825 | CAATACGTCGTCTCTGGCGTT | 3826 | 2769 | 2000 | 769 |
| 623 | MNGGRVL | 3827 | ATGAATGGGGGTCGTGTTCTG | 3828 | 2767 | 0 | 2767 |
| 624 | LSLTAGV | 3829 | CTGAGTTTGACTGCTGGGGTT | 3830 | 2764 | 0 | 2764 |
| 625 | FGIASGA | 3831 | TTTGGTATTGCTAGTGGGGCG | 3832 | 2763 | 763 | 2000 |
| 626 | EGGYSGA | 3833 | GAAGGGGGGTACTCAGGCGCG | 3834 | 2763 | 0 | 2763 |
| 627 | VTLGATS | 3835 | GTAACACTCGGAGCGACCAGC | 3836 | 2757 | 0 | 2757 |
| 628 | TGLQVGI | 3837 | ACTGGGCTGCAGGTTGGTATT | 3838 | 2756 | 2756 | 0 |
| 629 | IYPQSST | 3839 | ATATACCCGCAAAGCTCGACA | 3840 | 2756 | 0 | 2756 |
| 630 | NANSLME | 3841 | AATGCGAATTCTTTGATGGAG | 3842 | 2755 | 0 | 2755 |
| 631 | LHDGNTR | 3843 | TTGCATGATGGGAATACGCGG | 3844 | 2755 | 0 | 2755 |
| 632 | GYGSGLA | 3845 | GGTTATGGGTCGGGGCTGGCT | 3846 | 2755 | 0 | 2755 |
| 633 | TIGHSQV | 3847 | ACGATTGGTCATAGTCAGGTT | 3848 | 2754 | 2630 | 124 |
| 634 | VANSGLA | 3849 | GTGGCGAATTCTGGGCTGGCT | 3850 | 2754 | 2000 | 754 |
| 635 | AGLLNAL | 3851 | GCGGGGTTGCTGAATGCTTTG | 3852 | 2754 | 2000 | 754 |
| 636 | KNAGHVL | 3853 | AAGAATGCGGGTCATGTTCTG | 3854 | 2752 | 2000 | 752 |
| 637 | PMSNTHP | 3855 | CCGATGTCGAATACTCATCCG | 3856 | 2749 | 2749 | 0 |
| 638 | LAGSLPL | 3857 | CTTGCTGGTTCGCTTCCGTTG | 3858 | 2749 | 2000 | 749 |
| 639 | SRLENIS | 3859 | AGTAGGTTGGAGAATATTAGT | 3860 | 2748 | 0 | 2748 |
| 640 | HSEGVGR | 3861 | CATAGTGAGGGTGTTGGGCGG | 3862 | 2744 | 0 | 2744 |
| 641 | GAPINSF | 3863 | GGAGCACCAATAAACTCTTTC | 3864 | 2742 | 2000 | 742 |
| 642 | GLEPRVP | 3865 | GGGCTGGAGCCTCGTGTTCCT | 3866 | 2738 | 0 | 2738 |
| 643 | PAREGNF | 3867 | CCTGCCAGGGAAGGCAACTTC | 3868 | 2736 | 2000 | 736 |
| 644 | AAGGQVL | 3869 | GCTGCTGGGGGACAAGTCCTC | 3870 | 2735 | 2735 | 0 |
| 645 | TSYDKLV | 3871 | ACGTCGTATGATAAGTTGGTT | 3872 | 2734 | 1995 | 739 |
| 646 | ELNAVAR | 3873 | GAACTTAACGCAGTTGCTCGG | 3874 | 2733 | 2000 | 733 |
| 647 | SLRHVEV | 3875 | AGCCTACGCCACGTTGAAGTC | 3876 | 2725 | 2000 | 725 |
| 648 | AADSSGR | 3877 | GCGGCGGATAGTTCTGGGCGG | 3878 | 2724 | 2000 | 724 |
| 649 | ANEVKHV | 3879 | GCGAATGAGGTTAAGCATGTG | 3880 | 2724 | 2000 | 724 |
| 650 | DLAQSGR | 3881 | GATTTGGCTCAGAGTGGGCGT | 3882 | 2723 | 2000 | 723 |
| 651 | GLAGTNT | 3883 | GGGCTGGCTGGGACGAATACT | 3884 | 2722 | 2002 | 720 |
| 652 | SPSIGPV | 3885 | TCGCCTTCTATTGGTCCTGTG | 3886 | 2721 | 721 | 2000 |
| 653 | SVAGLSR | 3887 | TCTGTTGCTGGTCTTTCTAGG | 3888 | 2721 | 0 | 2721 |
| 654 | ADVHVKV | 3889 | GCGGATGTTCATGTGAAGGTG | 3890 | 2720 | 2720 | 0 |
| 655 | IVKQGDI | 3891 | ATTGTTAAGCAGGGTGATATT | 3892 | 2720 | 2720 | 0 |
| 656 | ASASGVA | 3893 | GCTTCTGCGTCGGGGGTGGCT | 3894 | 2719 | 0 | 2719 |
| 657 | MGGGNIP | 3895 | ATGGGAGGTGGCAACATACCC | 3896 | 2717 | 0 | 2717 |
| 658 | TPTSSTR | 3897 | ACACCAACATCCAGCACACGA | 3898 | 2716 | 2000 | 716 |
| 659 | VAEKAMA | 3899 | GTAGCTGAAAAAGCTATGGCA | 3900 | 2716 | 0 | 2716 |
| 660 | GSGSGAA | 3901 | GGTTCTGGGTCGGGGGCGGCT | 3902 | 2715 | 2000 | 715 |
| 661 | NMQDGGM | 3903 | AACATGCAAGACGGCGGCATG | 3904 | 2714 | 1356 | 1358 |
| 662 | QLRDNKT | 3905 | CAATTACGCGACAACAAAACG | 3906 | 2713 | 2000 | 713 |
| 663 | HAGLGVI | 3907 | CATGCTGGTCTTGGTGTTATT | 3908 | 2713 | 0 | 2713 |
| 664 | VNVSYRA | 3909 | GTGAATGTTTCGTATCGTGCT | 3910 | 2712 | 1101 | 1611 |
| 665 | IRNDKGP | 3911 | ATCCGAAACGACAAAGGGCCT | 3912 | 2711 | 2711 | 0 |
| 666 | ASLAQAV | 3913 | GCTAGTCTTGCACAAGCAGTT | 3914 | 2710 | 2000 | 710 |
| 667 | TLSHAEL | 3915 | ACGTTGTCTCATGCTGAGCTG | 3916 | 2710 | 2000 | 710 |
| 668 | TYSDGST | 3917 | ACCTACTCTGACGGTTCTACC | 3918 | 2710 | 0 | 2710 |
| 669 | REKGVTV | 3919 | AGAGAAAAAGGGGTCACGGTC | 3920 | 2709 | 2000 | 709 |
| 670 | ENRVYSP | 3921 | GAGAATCGTGTTTATTCTCCG | 3922 | 2707 | 0 | 2707 |
| 671 | ATQGTLR | 3923 | GCTACTCAGGGGACGCTTCGG | 3924 | 2706 | 2000 | 706 |
| 672 | KHVDTGA | 3925 | AAGCATGTGGATACGGGGGCG | 3926 | 2704 | 0 | 2704 |
| 673 | LANKMSD | 3927 | CTGGCTAATAAGATGAGTGAT | 3928 | 2701 | 701 | 2000 |
| 674 | TLVGVVS | 3929 | ACCCTCGTGGGGGTAGTCTCT | 3930 | 2699 | 0 | 2699 |
| 675 | GVPGTNS | 3931 | GGTGTTCCCGGTACTAACTCC | 3932 | 2696 | 2000 | 696 |
| 676 | RTDGADL | 3933 | CGTACGGATGGTGCGGATCTT | 3934 | 2696 | 0 | 2696 |
| 677 | PTQGTLL | 3935 | CCGACACAAGGAACTTTGTTG | 3936 | 2691 | 2000 | 691 |
| 678 | SHASDTK | 3937 | TCTCATGCTTCTGATACGAAG | 3938 | 2690 | 2690 | 0 |
| 679 | AVVSAGP | 3939 | GCGGTGGTGTCTGCTGGGCCG | 3940 | 2687 | 2000 | 687 |
| 680 | HNPQSLG | 3941 | CACAACCCTCAATCTCTCGGT | 3942 | 2686 | 0 | 2686 |
| 681 | TSLGIML | 3943 | ACCAGCCTAGGAATAATGCTT | 3944 | 2683 | 0 | 2683 |
| 682 | RPQGSES | 3945 | CGTCCTCAGGGTAGTGAGAGT | 3946 | 2682 | 1880 | 802 |
| 683 | AVNNVTL | 3947 | GCTGTGAATAATGTTACTCTT | 3948 | 2680 | 2680 | 0 |
| 684 | NGSAGNR | 3949 | AACGGTAGCGCTGGGAACCGC | 3950 | 2679 | 2679 | 0 |
| 685 | VFGETRA | 3951 | GTTTTTGGTGAGACGCGTGCG | 3952 | 2676 | 2000 | 676 |
| 686 | ASESSPS | 3953 | GCATCGGAATCAAGCCCATCT | 3954 | 2675 | 0 | 2675 |
| 687 | NSVGASI | 3955 | AACAGTGTAGGAGCGTCAATC | 3956 | 2674 | 0 | 2674 |
| 688 | DAGNQMG | 3957 | GATGCGGGGAATCAGATGGGG | 3958 | 2672 | 2672 | 0 |
| 689 | GSGRDGL | 3959 | GGTAGTGGAAGAGACGGACTG | 3960 | 2671 | 0 | 2671 |
| 690 | STVTGGP | 3961 | AGTACTGTTACTGGGGGTCCG | 3962 | 2671 | 0 | 2671 |
| 691 | TSYDKMV | 3963 | ACGTCGTATGATAAGATGGTT | 3964 | 2670 | 2345 | 325 |
| 692 | SGANLSN | 3965 | TCTGGTGCGAATTTGTCTAAT | 3966 | 2668 | 2009 | 659 |
| 693 | VKSTEGT | 3967 | GTTAAATCCACGGAAGGAACA | 3968 | 2666 | 2666 | 0 |
| 694 | GKDGHQM | 3969 | GGGAAGGATGGTCATCAGATG | 3970 | 2666 | 0 | 2666 |
| 695 | LGQKAGV | 3971 | CTTGGCCAAAAAGCAGGAGTC | 3972 | 2663 | 2663 | 0 |
| 696 | REQQKIW | 3973 | AGAGAACAACAAAAAATATGG | 3974 | 2663 | 2000 | 663 |
| 697 | AGNLSVK | 3975 | GCGGGGAATCTGAGTGTGAAG | 3976 | 2661 | 2661 | 0 |
| 698 | SHSLIEV | 3977 | TCTCATAGTCTGATTGAGGTG | 3978 | 2661 | 0 | 2661 |
| 699 | HVSGASL | 3979 | CATGTTTCGGGTGCTTCTCTT | 3980 | 2659 | 0 | 2659 |
| 700 | LNLKGVV | 3981 | TTGAATCTGAAGGGTGTGGTT | 3982 | 2657 | 2005 | 653 |
| 701 | AHEARGD | 3983 | GCGCACGAAGCACGAGGGGAC | 3984 | 2657 | 1883 | 774 |
| 702 | HANTAGV | 3985 | CATGCGAATACTGCTGGGGTG | 3986 | 2656 | 0 | 2656 |
| 703 | TGAGGHP | 3987 | ACGGGTGCTGGTGGGCATCCT | 3988 | 2654 | 0 | 2654 |
| 704 | LSRGQEM | 3989 | CTGTCACGAGGGCAAGAAATG | 3990 | 2653 | 2000 | 653 |
| 705 | LSNHGHV | 3991 | CTGAGTAATCATGGGCATGTT | 3992 | 2652 | 2000 | 652 |
| 706 | STHHTST | 3993 | TCTACACACCACACCTCAACC | 3994 | 2651 | 2000 | 651 |
| 707 | QKISTVQ | 3995 | CAGAAGATTTCGACTGTGCAG | 3996 | 2651 | 1536 | 1115 |
| 708 | GVRNTNV | 3997 | GGAGTCCGGAACACAAACGTA | 3998 | 2648 | 2648 | 0 |
| 709 | LTVSLNK | 3999 | CTAACTGTATCTCTTAACAAA | 4000 | 2647 | 2000 | 647 |
| 710 | LSNNGPV | 4001 | CTCTCTAACAACGGCCCCGTG | 4002 | 2645 | 2000 | 645 |
| 711 | LNLKGVV | 4003 | CTTAACCTCAAAGGGGTCGTC | 4004 | 2645 | 1896 | 749 |
| 712 | PKPSHGE | 4005 | CCGAAGCCTAGTCATGGTGAG | 4006 | 2645 | 0 | 2645 |
| 713 | ETNRGSV | 4007 | GAAACAAACCGGGGATCCGTA | 4008 | 2644 | 2000 | 644 |
| 714 | NLTSDKV | 4009 | AATCTGACGTCTGATAAGGTT | 4010 | 2642 | 2000 | 642 |
| 715 | MEDRART | 4011 | ATGGAGGATAGGGCTCGGACT | 4012 | 2642 | 0 | 2642 |
| 716 | GVAGTNS | 4013 | GGGGTGGCTGGGACGAATTCT | 4014 | 2641 | 2010 | 631 |
| 717 | SLGQDKL | 4015 | AGTCTAGGCCAAGACAAATTG | 4016 | 2640 | 2640 | 0 |
| 718 | GVGEGRA | 4017 | GGCGTCGGGGAAGGACGAGCC | 4018 | 2640 | 2000 | 640 |
| 719 | ASLLPPT | 4019 | GCATCGCTCTTGCCCCCCACG | 4020 | 2639 | 0 | 2639 |
| 720 | TALVLHK | 4021 | ACGGCTCTTGTTCTTCATAAG | 4022 | 2638 | 0 | 2638 |
| 721 | AVSDHTV | 4023 | GCTGTTAGTGATCATACTGTG | 4024 | 2637 | 2637 | 0 |
| 722 | SQSAIPN | 4025 | AGTCAGTCGGCTATTCCTAAT | 4026 | 2636 | 0 | 2636 |
| 723 | MNGGHLQ | 4027 | ATGAATGGGGGTCATCTTCAG | 4028 | 2635 | 2635 | 0 |
| 724 | KNGGNVL | 4029 | AAAAACGGTGGAAACGTGTTG | 4030 | 2635 | 2000 | 635 |
| 725 | GAVSSTT | 4031 | GGTGCTGTTTCTTCGACTACG | 4032 | 2633 | 2000 | 633 |
| 726 | STLNTST | 4033 | AGTACTCTTAATACTTCGACT | 4034 | 2631 | 2000 | 631 |
| 727 | RSPNVGQ | 4035 | AGGTCGCCTAATGTTGGGCAG | 4036 | 2630 | 2000 | 630 |
| 728 | PTQGTFR | 4037 | CCTACTCAGGGGACGTTTCGG | 4038 | 2630 | 1831 | 799 |
| 729 | NHGSDSK | 4039 | AACCACGGGTCAGACAGCAAA | 4040 | 2628 | 2000 | 628 |
| 730 | LPSGHLH | 4041 | CTTCCGAGTGGTCATCTTCAT | 4042 | 2628 | 2000 | 628 |
| 731 | SEKVVAT | 4043 | TCGGAGAAGGTGGTGGCGACG | 4044 | 2625 | 2000 | 625 |
| 732 | TVPNTVL | 4045 | ACCGTTCCCAACACAGTCCTG | 4046 | 2625 | 0 | 2625 |
| 733 | LSIGQGH | 4047 | CTATCCATAGGACAAGGACAC | 4048 | 2625 | 0 | 2625 |
| 734 | TSVLSQV | 4049 | ACGAGTGTTCTTTCGCAGGTT | 4050 | 2624 | 2000 | 624 |
| 735 | VLSSHGP | 4051 | GTCTTATCGTCACACGGCCCA | 4052 | 2624 | 0 | 2624 |
| 736 | TSQASSV | 4053 | ACGTCGCAGGCTTCGTCTGTG | 4054 | 2623 | 2000 | 623 |
| 737 | NRSAGDR | 4055 | AACCGTTCGGCTGGCGACCGA | 4056 | 2622 | 2000 | 622 |
| 738 | RGGVTTQ | 4057 | CGAGGAGGAGTAACAACACAA | 4058 | 2622 | 2000 | 622 |
| 739 | SGLKGVN | 4059 | TCCGGACTAAAAGGTGTTAAC | 4060 | 2622 | 0 | 2622 |
| 740 | STITNLM | 4061 | AGTACTATTACTAATCTGATG | 4062 | 2619 | 1884 | 735 |
| 741 | KGASVTL | 4063 | AAGGGGGCTAGTGTTACGCTT | 4064 | 2618 | 2618 | 0 |
| 742 | TSTHEGV | 4065 | ACTTCAACGCACGAAGGAGTT | 4066 | 2618 | 2000 | 618 |
| 743 | KLGGGVS | 4067 | AAGTTGGGTGGTGGGGTGTCG | 4068 | 2618 | 2000 | 618 |
| 744 | GLESRVP | 4069 | GGTTTAGAATCAAGAGTGCCC | 4070 | 2616 | 2616 | 0 |
| 745 | QSLSDGV | 4071 | CAATCATTAAGCGACGGCGTC | 4072 | 2614 | 2614 | 0 |
| 746 | ADAAHAL | 4073 | GCGGACGCCGCCCACGCGCTT | 4074 | 2613 | 0 | 2613 |
| 747 | CAGGCEL | 4075 | TGTGCGGGTGGTTGTGAGCTT | 4076 | 2612 | 2000 | 612 |
| 748 | PGERNNP | 4077 | CCAGGAGAACGTAACAACCCC | 4078 | 2612 | 1929 | 683 |
| 749 | GVRNTDI | 4079 | GGAGTTCGGAACACAGACATC | 4080 | 2612 | 0 | 2612 |
| 750 | STLHTSI | 4081 | AGTACTCTTCATACTTCGATT | 4082 | 2611 | 0 | 2611 |
| 751 | AEVGSNR | 4083 | GCAGAAGTTGGCTCAAACAGG | 4084 | 2610 | 0 | 2610 |
| 752 | NDRNTSS | 4085 | AACGACCGAAACACATCAAGT | 4086 | 2608 | 2000 | 608 |
| 753 | GPVSSTK | 4087 | GGTCCTGTTTCTTCGACTAAG | 4088 | 2608 | 2000 | 608 |
| 754 | VHGTGGA | 4089 | GTGCATGGTACTGGGGGTGCT | 4090 | 2608 | 1488 | 1120 |
| 755 | VLASSGP | 4091 | GTATTGGCAAGTTCGGGCCCA | 4092 | 2608 | 0 | 2608 |
| 756 | LAGSISL | 4093 | CTTGCTGGGTCGATTTCGTTG | 4094 | 2604 | 2000 | 604 |
| 757 | SRTLEET | 4095 | TCTAGGACGCTTGAGGAGACT | 4096 | 2604 | 2000 | 604 |
| 758 | KEIRVSV | 4097 | AAGGAGATTCGTGTGTCGGTT | 4098 | 2601 | 2000 | 601 |
| 759 | HAVAGAT | 4099 | CATGCTGTGGCTGGGGCGACT | 4100 | 2597 | 2000 | 597 |
| 760 | VDHGGVN | 4101 | GTAGACCACGGCGGGGTTAAC | 4102 | 2595 | 595 | 2000 |
| 761 | TTATIVR | 4103 | ACCACTGCTACGATCGTACGC | 4104 | 2594 | 594 | 2000 |
| 762 | SGEGLAS | 4105 | TCGGGTGAGGGGCTGGCTAGT | 4106 | 2593 | 2000 | 593 |
| 763 | TVGLTIA | 4107 | ACCGTAGGCCTTACTATAGCA | 4108 | 2592 | 1729 | 863 |
| 764 | DYDSGRR | 4109 | GACTACGACTCTGGACGTAGA | 4110 | 2589 | 0 | 2589 |
| 765 | QGVTVGL | 4111 | CAGGGGGTTACGGTTGGGCTT | 4112 | 2589 | 0 | 2589 |
| 766 | KSQSENS | 4113 | AAGTCGCAGTCGGAGAATAGT | 4114 | 2587 | 2587 | 0 |
| 767 | SEGLSRD | 4115 | TCCGAAGGGCTGTCCAGAGAC | 4116 | 2587 | 2000 | 587 |
| 768 | SGDGTSK | 4117 | TCGGGTGATGGGACTTCTAAG | 4118 | 2586 | 2000 | 586 |
| 769 | DGSAGDR | 4119 | GATGGGAGTGCGGGTGATCGT | 4120 | 2586 | 2000 | 586 |
| 770 | TDALTSK | 4121 | ACGGACGCACTCACAAGCAAA | 4122 | 2583 | 2000 | 583 |
| 771 | QSQTTVG | 4123 | CAGTCTCAGACGACTGTTGGT | 4124 | 2583 | 1923 | 660 |
| 772 | GSASAVA | 4125 | GGCAGCGCTTCAGCAGTAGCA | 4126 | 2583 | 0 | 2583 |
| 773 | VTVTMSR | 4127 | GTCACGGTAACCATGTCACGG | 4128 | 2581 | 0 | 2581 |
| 774 | GVGNTNI | 4129 | GGCGTAGGTAACACGAACATA | 4130 | 2579 | 2000 | 579 |
| 775 | TIAHSQV | 4131 | ACGATTGCTCATAGTCAGGTT | 4132 | 2579 | 0 | 2579 |
| 776 | VQGTQTG | 4133 | GTGCAGGGTACGCAGACTGGT | 4134 | 2578 | 2000 | 578 |
| 777 | LRVTEIL | 4135 | CTTCGAGTCACTGAAATACTA | 4136 | 2578 | 0 | 2578 |
| 778 | SSSGLVR | 4137 | AGTTCCTCTGGGCTAGTCCGA | 4138 | 2577 | 0 | 2577 |
| 779 | LSLSKDK | 4139 | CTATCGTTATCTAAAGACAAA | 4140 | 2574 | 0 | 2574 |
| 780 | TGISVNG | 4141 | ACCGGCATCTCTGTCAACGGT | 4142 | 2573 | 2000 | 573 |
| 781 | SKSAFPN | 4143 | TCGAAATCCGCCTTCCCAAAC | 4144 | 2571 | 2571 | 0 |
| 782 | VGNSSGV | 4145 | GTTGGTAATTCTTCGGGTGTG | 4146 | 2571 | 2000 | 571 |
| 783 | DSHVSGD | 4147 | GACTCACACGTCTCCGGCGAC | 4148 | 2569 | 1477 | 1092 |
| 784 | KVYDTPM | 4149 | AAGGTTTATGATACTCCTATG | 4150 | 2568 | 2000 | 568 |
| 785 | GSMENVR | 4151 | GGGAGTATGGAGAATGTGCGT | 4152 | 2567 | 2000 | 567 |
| 786 | ETTQGSP | 4153 | GAAACAACGCAAGGCAGTCCC | 4154 | 2565 | 2000 | 565 |
| 787 | KGSGLEI | 4155 | AAGGGGTCTGGGCTTGAGATT | 4156 | 2561 | 0 | 2561 |
| 788 | TALQVSI | 4157 | ACTGCGCTGCAGGTTAGTATT | 4158 | 2560 | 1976 | 584 |
| 789 | PTQSDLA | 4159 | CCTACGCAGTCGGATCTTGCT | 4160 | 2558 | 2000 | 558 |
| 790 | STQTLGE | 4161 | TCGACTCAGACTTTGGGGGAG | 4162 | 2558 | 1869 | 690 |
| 791 | LSNRGPV | 4163 | CTTTCTAACAGAGGCCCGGTG | 4164 | 2556 | 0 | 2556 |
| 792 | EHVNVKV | 4165 | GAGCATGTTAATGTGAAGGTG | 4166 | 2555 | 2000 | 555 |
| 793 | PLKGGGE | 4167 | CCTCTGAAGGGTGGTGGGGAG | 4168 | 2554 | 1776 | 778 |
| 794 | SHGSVSK | 4169 | TCTCATGGTTCTGTTTCGAAG | 4170 | 2552 | 0 | 2552 |
| 795 | MLGGGVS | 4171 | ATGTTGGGTGGTGGGGTGTCG | 4172 | 2549 | 1365 | 1184 |
| 796 | TAVHSTS | 4173 | ACTGCCGTACACAGCACGTCA | 4174 | 2548 | 2000 | 548 |
| 797 | QADSHGR | 4175 | CAAGCAGACAGCCACGGCCGT | 4176 | 2548 | 1815 | 733 |
| 798 | ATLKPDY | 4177 | GCAACGTTGAAACCCGACTAC | 4178 | 2548 | 0 | 2548 |
| 799 | LDTSARV | 4179 | CTTGATACTAGTGCTCGTGTT | 4180 | 2547 | 0 | 2547 |
| 800 | HTSGTSS | 4181 | CATACGAGTGGGACGTCGTCG | 4182 | 2544 | 2000 | 544 |
| 801 | TGARDQY | 4183 | ACAGGGGCGCGTGACCAATAC | 4184 | 2544 | 1585 | 959 |
| 802 | SGETLRL | 4185 | TCTGGGGAGACGCTTAGGCTT | 4186 | 2544 | 1566 | 978 |
| 803 | VSLSDGV | 4187 | GTAAGCCTTTCGGACGGTGTG | 4188 | 2542 | 2000 | 542 |
| 804 | AGVVNAL | 4189 | GCGGGGGTGGTGAATGCTTTG | 4190 | 2541 | 2231 | 310 |
| 805 | DASKLVN | 4191 | GATGCGAGTAAGCTTGTGAAT | 4192 | 2539 | 2000 | 539 |
| 806 | EIRLSTH | 4193 | GAAATAAGACTGTCCACCCAC | 4194 | 2539 | 2000 | 539 |
| 807 | MGRTDGL | 4195 | ATGGGGCGTACTGATGGGTTG | 4196 | 2536 | 1507 | 1029 |
| 808 | TGLLVSI | 4197 | ACTGGGCTGCTGGTTAGTATT | 4198 | 2536 | 0 | 2536 |
| 809 | KAGLLFD | 4199 | AAAGCAGGCCTTCTATTCGAC | 4200 | 2532 | 2000 | 532 |
| 810 | SRAEGIK | 4201 | AGTCGTGCGGAGGGGATTAAG | 4202 | 2532 | 2000 | 532 |
| 811 | NGKVDRD | 4203 | AATGGGAAGGTTGATCGGGAT | 4204 | 2530 | 2000 | 530 |
| 812 | SPPSSPR | 4205 | TCTCCGCCGAGTTCGCCGCGT | 4206 | 2530 | 1806 | 724 |
| 813 | NGIAGDR | 4207 | AATGGGATTGCGGGTGATCGT | 4208 | 2527 | 2000 | 527 |
| 814 | NRASDGI | 4209 | AACCGAGCTTCTGACGGGATA | 4210 | 2524 | 2000 | 524 |
| 815 | GVELISR | 4211 | GGTGTTGAGCTGATTTCGCGG | 4212 | 2521 | 2000 | 521 |
| 816 | RVQLSET | 4213 | CGGGTGCAGCTGTCTGAGACT | 4214 | 2521 | 2000 | 521 |
| 817 | LNYSVSL | 4215 | TTAAACTACAGTGTAAGCCTC | 4216 | 2520 | 1067 | 1454 |
| 818 | ASVSSKS | 4217 | GCTAGTGTGTCTTCGAAGAGT | 4218 | 2519 | 2000 | 519 |
| 819 | TPSTGVL | 4219 | ACTCCTAGTACTGGGGTGCTG | 4220 | 2519 | 1899 | 620 |
| 820 | HANTAGV | 4221 | CACGCAAACACAGCAGGTGTA | 4222 | 2518 | 2000 | 518 |
| 821 | VVSVLNV | 4223 | GTGGTGAGTGTGCTGAATGTT | 4224 | 2518 | 2000 | 518 |
| 822 | AHDHVKV | 4225 | GCGCATGATCATGTGAAGGTG | 4226 | 2518 | 1558 | 960 |
| 823 | SSEGRNV | 4227 | TCGTCAGAAGGAAGAAACGTT | 4228 | 2517 | 2000 | 517 |
| 824 | NNNGATS | 4229 | AATAATAATGGTGCGACTTCT | 4230 | 2516 | 1571 | 945 |
| 825 | LSQGSQQ | 4231 | CTGTCTCAGGGGTCGCAGCAG | 4232 | 2516 | 5 | 2511 |
| 826 | GGTTSGH | 4233 | GGCGGGACAACGAGCGGGCAC | 4234 | 2515 | 0 | 2515 |
| 827 | TLASQEL | 4235 | ACTCTGGCGTCGCAGGAGCTG | 4236 | 2514 | 2000 | 514 |
| 828 | ATGTESR | 4237 | GCGACTGGTACTGAGTCGCGG | 4238 | 2510 | 2000 | 510 |
| 829 | NNANLVI | 4239 | AATAATGCTAATCTGGTTATT | 4240 | 2510 | 2000 | 510 |
| 830 | STITTLK | 4241 | TCCACAATAACGACACTTAAA | 4242 | 2509 | 0 | 2509 |
| 831 | QLNSADS | 4243 | CAGCTGAATTCGGCTGATAGT | 4244 | 2507 | 1374 | 1133 |
| 832 | ETNFGVS | 4245 | GAGACTAATTTTGGGGTGTCT | 4246 | 2505 | 0 | 2505 |
| 833 | ENVHVKV | 4247 | GAAAACGTGCACGTTAAAGTC | 4248 | 2500 | 2000 | 500 |
| 834 | AGGGAPM | 4249 | GCCGGCGGAGGAGCACCCATG | 4250 | 2500 | 2000 | 500 |
| 835 | PNESVRA | 4251 | CCTAACGAAAGTGTACGGGCA | 4252 | 2498 | 1811 | 687 |
| 836 | LKPGLAD | 4253 | CTGAAGCCGGGGTTGGCGGAT | 4254 | 2498 | 0 | 2498 |
| 837 | LANRLSV | 4255 | CTGGCTAATAGGTTGAGTGTT | 4256 | 2497 | 1615 | 882 |
| 838 | FAGIAQA | 4257 | TTTGCGGGGATTGCGCAGGCG | 4258 | 2493 | 2000 | 493 |
| 839 | RVHSAQH | 4259 | CGTGTCCACAGTGCTCAACAC | 4260 | 2491 | 1933 | 558 |
| 840 | LSGLRSG | 4261 | TTGTCTGGTCTTCGTAGTGGT | 4262 | 2490 | 2000 | 490 |
| 841 | SEGLSRL | 4263 | TCCGAAGGATTATCCCGACTT | 4264 | 2490 | 2000 | 490 |
| 842 | RLLRDES | 4265 | AGGTTGCTACGAGACGAATCT | 4266 | 2490 | 0 | 2490 |
| 843 | SPSAIPN | 4267 | TCTCCCAGTGCGATACCGAAC | 4268 | 2488 | 0 | 2488 |
| 844 | VSRGAEL | 4269 | GTTTCGAGGGGTGCGGAGCTG | 4270 | 2488 | 0 | 2488 |
| 845 | KGSDTPM | 4271 | AAAGGATCGGACACACCGATG | 4272 | 2487 | 2000 | 487 |
| 846 | VDHGGVM | 4273 | GTGGATCATGGTGGTGTGATG | 4274 | 2487 | 2000 | 487 |
| 847 | VNAALGI | 4275 | GTTAATGCTGCGCTTGGGATT | 4276 | 2487 | 2000 | 487 |
| 848 | TMSMGKL | 4277 | ACGATGTCGATGGGGAAGCTG | 4278 | 2484 | 2000 | 484 |
| 849 | INGGHDL | 4279 | ATCAACGGAGGCCACGACCTC | 4280 | 2484 | 2000 | 484 |
| 850 | GTGSTIV | 4281 | GGGACGGGTTCTACGATTGTG | 4282 | 2484 | 2000 | 484 |
| 851 | TNGGHVL | 4283 | ACTAACGGCGGACACGTGCTC | 4284 | 2484 | 1444 | 1040 |
| 852 | LISSTMR | 4285 | TTGATTTCTAGTACGATGCGT | 4286 | 2484 | 1275 | 1209 |
| 853 | AGGNGSY | 4287 | GCAGGAGGAAACGGCTCCTAC | 4288 | 2483 | 1618 | 865 |
| 854 | ITQAAYV | 4289 | ATTACGCAGGCTGCTTATGTT | 4290 | 2482 | 2000 | 482 |
| 855 | AVLAGSM | 4291 | GCGGTTCTGGCGGGGTCTATG | 4292 | 2480 | 2000 | 480 |
| 856 | GASGAVL | 4293 | GGAGCTTCAGGGGCGGTCCTC | 4294 | 2480 | 0 | 2480 |
| 857 | PTATESL | 4295 | CCTACCGCTACAGAAAGTCTC | 4296 | 2480 | 0 | 2480 |
| 858 | SPQGGLP | 4297 | TCGCCGCAGGGGGGTCTTCCT | 4298 | 2477 | 2000 | 477 |
| 859 | VRASIVD | 4299 | GTGAGGGCTAGTATTGTTGAT | 4300 | 2477 | 2000 | 477 |
| 860 | AVKENET | 4301 | GCGGTGAAGGAGAATGAGACG | 4302 | 2477 | 2000 | 477 |
| 861 | HVSQDHS | 4303 | CATGTGTCTCAGGATCATTCG | 4304 | 2477 | 6 | 2471 |
| 862 | SKSNDSS | 4305 | AGTAAGTCTAATGATAGTTCT | 4306 | 2477 | 0 | 2477 |
| 863 | KTEQVQP | 4307 | AAAACAGAACAAGTCCAACCT | 4308 | 2476 | 0 | 2476 |
| 864 | MTGTAHQ | 4309 | ATGACGGGTACGGCTCATCAG | 4310 | 2475 | 1832 | 643 |
| 865 | IKQAVYV | 4311 | ATAAAACAAGCAGTCTACGTA | 4312 | 2474 | 2000 | 474 |
| 866 | IPSGGPR | 4313 | ATTCCGTCTGGGGGTCCGCGT | 4314 | 2474 | 2000 | 474 |
| 867 | TKPNMVS | 4315 | ACTAAGCCGAATATGGTGAGT | 4316 | 2472 | 2000 | 472 |
| 868 | NDRNTSS | 4317 | AATGATAGGAATACGTCTTCG | 4318 | 2471 | 2000 | 471 |
| 869 | TGISVGK | 4319 | ACCGGGATATCAGTAGGCAAA | 4320 | 2471 | 1429 | 1042 |
| 870 | SEQQKDW | 4321 | TCTGAACAACAAAAAGACTGG | 4322 | 2470 | 2000 | 470 |
| 871 | LLTSVKV | 4323 | CTACTAACGTCTGTTAAAGTA | 4324 | 2467 | 1938 | 529 |
| 872 | TTEKVTG | 4325 | ACGACTGAGAAGGTTACTGGT | 4326 | 2464 | 0 | 2464 |
| 873 | GALSTTK | 4327 | GGAGCGTTAAGTACAACCAAA | 4328 | 2463 | 2000 | 463 |
| 874 | RTLHDNT | 4329 | AGGACCCTCCACGACAACACA | 4330 | 2463 | 2000 | 463 |
| 875 | TREHNSI | 4331 | ACTAGGGAGCATAATTCGATT | 4332 | 2463 | 2000 | 463 |
| 876 | NHITGGV | 4333 | AACCACATAACAGGCGGGGTC | 4334 | 2463 | 1819 | 644 |
| 877 | SQAQAGY | 4335 | TCTCAGGCGCAGGCGGGTTAT | 4336 | 2462 | 2000 | 462 |
| 878 | NTSRIGV | 4337 | AATACGAGTAGGATTGGTGTG | 4338 | 2462 | 1546 | 916 |
| 879 | HVASTAA | 4339 | CACGTAGCGTCGACCGCGGCT | 4340 | 2461 | 2000 | 461 |
| 880 | IARINSH | 4341 | ATAGCCAGAATCAACTCCCAC | 4342 | 2459 | 0 | 2459 |
| 881 | LISSTLR | 4343 | TTGATTTCTAGTACGCTGCGT | 4344 | 2459 | 0 | 2459 |
| 882 | SRLENIV | 4345 | TCGCGGCTCGAAAACATAGTA | 4346 | 2458 | 458 | 2000 |
| 883 | SPTSSPT | 4347 | TCTCCGACGAGTTCGCCGACT | 4348 | 2456 | 0 | 2456 |
| 884 | HDGLGVI | 4349 | CATGATGGTCTTGGTGTTATT | 4350 | 2455 | 2000 | 455 |
| 885 | SDQNGPR | 4351 | TCTGATCAGAATGGGCCTCGG | 4352 | 2454 | 2000 | 454 |
| 886 | SEQKNVW | 4353 | AGTGAGCAGAAGAATGTTTGG | 4354 | 2454 | 0 | 2454 |
| 887 | RIVVSVP | 4355 | CGGATAGTTGTGAGCGTACCC | 4356 | 2452 | 0 | 2452 |
| 888 | QDGPAVK | 4357 | CAGGATGGGCCTGCGGTGAAG | 4358 | 2450 | 0 | 2450 |
| 889 | KKVITDD | 4359 | AAGAAGGTGATTACTGATGAT | 4360 | 2449 | 2000 | 449 |
| 890 | MGGVNNT | 4361 | ATGGGGGGGGTTAATAATACG | 4362 | 2448 | 0 | 2448 |
| 891 | VSSKGEW | 4363 | GTCAGCAGCAAAGGTGAATGG | 4364 | 2447 | 2000 | 447 |
| 892 | GSGQMDA | 4365 | GGGAGTGGGCAGATGGATGCT | 4366 | 2444 | 0 | 2444 |
| 893 | MAGKAPP | 4367 | ATGGCTGGGAAGGCGCCGCCG | 4368 | 2443 | 0 | 2443 |
| 894 | QVRDTMT | 4369 | CAAGTTCGAGACACAATGACC | 4370 | 2442 | 2000 | 442 |
| 895 | GHVTSGD | 4371 | GGCCACGTCACATCTGGGGAC | 4372 | 2442 | 2000 | 442 |
| 896 | SEVSKGI | 4373 | AGTGAGGTTAGTAAGGGTATT | 4374 | 2442 | 2000 | 442 |
| 897 | NRQEHTY | 4375 | AATCGGCAGGAGCATACGTAT | 4376 | 2442 | 0 | 2442 |
| 898 | YVSTVVG | 4377 | TATGTTAGTACTGTTGTGGGG | 4378 | 2441 | 2000 | 441 |
| 899 | SNLSVVI | 4379 | TCTAACCTCTCAGTCGTAATC | 4380 | 2440 | 2000 | 440 |
| 900 | GHPQTTA | 4381 | GGGCATCCTCAGACTACGGCT | 4382 | 2440 | 2000 | 440 |
| 901 | ESSGNKL | 4383 | GAGTCTAGTGGTAATAAGCTG | 4384 | 2439 | 2000 | 439 |
| 902 | NFQADGL | 4385 | AACTTCCAAGCAGACGGACTG | 4386 | 2439 | 2000 | 439 |
| 903 | GTSTLGY | 4387 | GGCACTTCAACCCTCGGCTAC | 4388 | 2438 | 2000 | 438 |
| 904 | MNVDGRD | 4389 | ATGAATGTTGATGGGCGTGAT | 4390 | 2438 | 2000 | 438 |
| 905 | QAIEGNF | 4391 | CAAGCCATCGAAGGGAACTTC | 4392 | 2438 | 1895 | 543 |
| 906 | KSHSENN | 4393 | AAGTCGCATTCGGAGAATAAT | 4394 | 2438 | 0 | 2438 |
| 907 | VNYSVAL | 4395 | GTAAACTACAGTGTTGCACTC | 4396 | 2438 | 0 | 2438 |
| 908 | SGSRITV | 4397 | TCGGGTAGTCGGATTACTGTT | 4398 | 2437 | 0 | 2437 |
| 909 | LSLNDGD | 4399 | CTATCACTTAACGACGGCGAC | 4400 | 2436 | 2000 | 436 |
| 910 | QGGNSGA | 4401 | CAAGGGGGGAACTCGGGCGCA | 4402 | 2435 | 2435 | 0 |
| 911 | LSNMLTV | 4403 | CTGTCTAATATGTTGACTGTT | 4404 | 2435 | 2000 | 435 |
| 912 | AGGGGPR | 4405 | GCAGGCGGAGGCGGACCACGT | 4406 | 2434 | 2000 | 434 |
| 913 | FDKTGVH | 4407 | TTTGATAAGACGGGGGTGCAT | 4408 | 2434 | 2000 | 434 |
| 914 | GRSQLQM | 4409 | GGGCGGTCGCAGTTGCAGATG | 4410 | 2433 | 2000 | 433 |
| 915 | VNAGHGI | 4411 | GTTAATGCTGGGCATGGGATT | 4412 | 2433 | 2000 | 433 |
| 916 | RILQSGV | 4413 | CGGATACTCCAATCGGGTGTG | 4414 | 2432 | 2000 | 432 |
| 917 | RTLGIPS | 4415 | CGGACGTTGGGGATTCCTTCT | 4416 | 2432 | 0 | 2432 |
| 918 | LGGLGGL | 4417 | CTAGGAGGGCTGGGAGGCTTA | 4418 | 2431 | 2000 | 431 |
| 919 | GIVGSVP | 4419 | GGGATTGTGGGTAGTGTTCCG | 4420 | 2427 | 0 | 2427 |
| 920 | SSDRLLA | 4421 | TCTAGCGACAGACTCTTAGCG | 4422 | 2427 | 0 | 2427 |
| 921 | KDVVRGS | 4423 | AAGGATGTGGTGCGGGGTAGT | 4424 | 2426 | 2000 | 426 |
| 922 | LGHSAEP | 4425 | CTGGGACACTCAGCAGAACCC | 4426 | 2426 | 2000 | 426 |
| 923 | STIPNLM | 4427 | AGTACTATTCCTAATCTGATG | 4428 | 2426 | 0 | 2426 |
| 924 | GGTIGGH | 4429 | GGGGGTACGATTGGTGGTCAT | 4430 | 2425 | 2000 | 425 |
| 925 | VPAGLGR | 4431 | GTTCCCGCAGGCTTAGGCCGT | 4432 | 2425 | 0 | 2425 |
| 926 | LVHTTNN | 4433 | CTAGTCCACACGACCAACAAC | 4434 | 2424 | 2000 | 424 |
| 927 | MTSGNLM | 4435 | ATGACTTCCGGCAACCTCATG | 4436 | 2424 | 0 | 2424 |
| 928 | KESLSGS | 4437 | AAGGAGTCGCTTTCGGGTTCT | 4438 | 2423 | 1377 | 1046 |
| 929 | LRVTEIL | 4439 | TTGCGTGTGACGGAGATTCTG | 4440 | 2421 | 2000 | 421 |
| 930 | GHNVGVH | 4441 | GGTCATAATGTTGGTGTTCAT | 4442 | 2419 | 2000 | 419 |
| 931 | LDKVRPA | 4443 | TTGGATAAGGTGCGTCCGGCG | 4444 | 2419 | 2000 | 419 |
| 932 | LVANTPT | 4445 | CTCGTCGCAAACACACCAACC | 4446 | 2418 | 0 | 2418 |
| 933 | SHGSDYK | 4447 | TCACACGGATCCGACTACAAA | 4448 | 2417 | 2274 | 143 |
| 934 | TVHAPGT | 4449 | ACCGTTCACGCGCCAGGCACT | 4450 | 2417 | 2000 | 417 |
| 935 | LNGGHVM | 4451 | CTCAACGGCGGACACGTGATG | 4452 | 2417 | 2000 | 417 |
| 936 | LTLSTGV | 4453 | CTTACGCTGAGTACTGGGGTG | 4454 | 2417 | 2000 | 417 |
| 937 | VVQVNGR | 4455 | GTTGTTCAGGTTAATGGGCGT | 4456 | 2416 | 2007 | 408 |
| 938 | RNGVTSS | 4457 | AGAAACGGAGTAACGAGTTCG | 4458 | 2416 | 2000 | 416 |
| 939 | GSASGEA | 4459 | GGCTCCGCTTCCGGAGAAGCC | 4460 | 2415 | 2000 | 415 |
| 940 | STQAVYV | 4461 | AGTACGCAGGCTGTTTATGTT | 4462 | 2415 | 0 | 2415 |
| 941 | SVDNGKR | 4463 | TCAGTCGACAACGGCAAACGA | 4464 | 2415 | 0 | 2415 |
| 942 | TLHDKVL | 4465 | ACTCTGCATGATAAGGTGTTG | 4466 | 2414 | 2000 | 414 |
| 943 | NDVRGSN | 4467 | AACGACGTCAGAGGGTCCAAC | 4468 | 2414 | 414 | 2000 |
| 944 | HMNITVS | 4469 | CATATGAATATTACGGTTTCG | 4470 | 2413 | 1975 | 438 |
| 945 | TTAAIIR | 4471 | ACGACGGCGGCTATTATTAGG | 4472 | 2413 | 0 | 2413 |
| 946 | VDHGGVV | 4473 | GTGGATCATGGTGGTGTGGTT | 4474 | 2412 | 2000 | 412 |
| 947 | QGGYSGV | 4475 | CAAGGGGGATACTCTGGTGTT | 4476 | 2412 | 2000 | 412 |
| 948 | EPVASTI | 4477 | GAGCCTGTGGCTTCTACTATT | 4478 | 2411 | 2000 | 411 |
| 949 | LGDTAYS | 4479 | TTAGGCGACACCGCTTACTCA | 4480 | 2410 | 0 | 2410 |
| 950 | NGSAGDH | 4481 | AACGGCTCGGCTGGAGACCAC | 4482 | 2409 | 2000 | 409 |
| 951 | TAVHTTS | 4483 | ACCGCAGTTCACACCACATCC | 4484 | 2409 | 1911 | 498 |
| 952 | TGARDQY | 4485 | ACTGGTGCTCGGGATCAGTAT | 4486 | 2409 | 1190 | 1219 |
| 953 | NGSAGDH | 4487 | AATGGGAGTGCGGGTGATCAT | 4488 | 2408 | 2006 | 401 |
| 954 | LAGMGGI | 4489 | CTTGCGGGTATGGGGGGGATT | 4490 | 2407 | 934 | 1474 |
| 955 | THRDAGV | 4491 | ACTCATAGGGATGCTGGTGTG | 4492 | 2405 | 2000 | 405 |
| 956 | YREMGGS | 4493 | TACAGAGAAATGGGCGGCTCC | 4494 | 2405 | 2000 | 405 |
| 957 | ETNLYHA | 4495 | GAGACGAATTTGTATCATGCT | 4496 | 2404 | 2000 | 404 |
| 958 | SGANSSN | 4497 | TCTGGTGCGAATTCGTCTAAT | 4498 | 2402 | 2000 | 402 |
| 959 | IVNSREF | 4499 | ATTGTGAATAGTCGTGAGTTT | 4500 | 2402 | 1418 | 984 |
| 960 | ESLGGPR | 4501 | GAATCCTTGGGAGGCCCTCGA | 4502 | 2402 | 0 | 2402 |
| 961 | VVDSYNK | 4503 | GTTGTTGATTCGTATAATAAG | 4504 | 2401 | 2000 | 401 |
| 962 | GGVSSTN | 4505 | GGAGGAGTCTCGTCTACCAAC | 4506 | 2400 | 2000 | 400 |
| 963 | DALTRLA | 4507 | GATGCTCTGACGCGGTTGGCG | 4508 | 2400 | 2000 | 400 |
| 964 | SLRAGVP | 4509 | AGCCTAAGAGCAGGTGTACCG | 4510 | 2400 | 0 | 2400 |
| 965 | MGWGTNP | 4511 | ATGGGGTGGGGTACTAATCCG | 4512 | 2399 | 0 | 2399 |
| 966 | VESGSLG | 4513 | GTTGAGAGTGGTTCTCTTGGG | 4514 | 2398 | 2000 | 398 |
| 967 | QGGYSLG | 4515 | CAAGGGGGATACAGCTTAGGA | 4516 | 2397 | 1800 | 597 |
| 968 | DSKDVHR | 4517 | GATAGTAAGGATGTTCATAGG | 4518 | 2397 | 0 | 2397 |
| 969 | SANPVAR | 4519 | TCTGCGAATCCGGTTGCGAGG | 4520 | 2396 | 2000 | 396 |
| 970 | SILSGVS | 4521 | TCAATATTATCAGGGGTATCC | 4522 | 2396 | 2000 | 396 |
| 971 | HAADVQR | 4523 | CATGCGGCGGATGTGCAGAGG | 4524 | 2396 | 2000 | 396 |
| 972 | LSRGAEK | 4525 | CTTTCGAGGGGTGCGGAGAAG | 4526 | 2395 | 2000 | 395 |
| 973 | AEGGAPR | 4527 | GCAGAAGGGGGCGCCCCTCGG | 4528 | 2395 | 1733 | 662 |
| 974 | SRNGNVV | 4529 | TCTCGGAATGGGAATGTTGTT | 4530 | 2395 | 0 | 2395 |
| 975 | LVATTLS | 4531 | CTGGTTGCAACTACCCTTTCT | 4532 | 2394 | 2000 | 394 |
| 976 | AHGHVKV | 4533 | GCGCATGGTCATGTGAAGGTG | 4534 | 2394 | 2000 | 394 |
| 977 | NAGVAQA | 4535 | AACGCCGGAGTAGCCCAAGCA | 4536 | 2394 | 2000 | 394 |
| 978 | NRDNVAF | 4537 | AATCGGGATAATGTTGCTTTT | 4538 | 2394 | 0 | 2394 |
| 979 | SVHSGLL | 4539 | AGTGTTCATAGTGGGCTGCTT | 4540 | 2393 | 2000 | 393 |
| 980 | RQMGITV | 4541 | CGGCAGATGGGTATTACTGTT | 4542 | 2393 | 2000 | 393 |
| 981 | LSHIGGL | 4543 | CTATCACACATAGGAGGCCTA | 4544 | 2392 | 2000 | 392 |
| 982 | GTVTLGY | 4545 | GGGACGGTGACTTTGGGGTAT | 4546 | 2391 | 2000 | 391 |
| 983 | SYGDGGV | 4547 | TCTTACGGAGACGGAGGAGTG | 4548 | 2391 | 2000 | 391 |
| 984 | NHVSGSS | 4549 | AATCATGTTTCTGGTAGTTCT | 4550 | 2391 | 1253 | 1138 |
| 985 | GSRALSS | 4551 | GGATCACGTGCCCTGTCAAGT | 4552 | 2390 | 2000 | 390 |
| 986 | GLNHVGL | 4553 | GGCCTCAACCACGTGGGCCTT | 4554 | 2389 | 2000 | 389 |
| 987 | TLTNGMP | 4555 | ACGCTTACTAATGGGATGCCT | 4556 | 2389 | 1699 | 690 |
| 988 | MGASVTH | 4557 | ATGGGAGCGTCCGTGACTCAC | 4558 | 2386 | 1864 | 522 |
| 989 | VQQLAIK | 4559 | GTGCAGCAGTTGGCGATTAAG | 4560 | 2385 | 0 | 2385 |
| 990 | LSNLSNG | 4561 | CTTAGTAATCTGTCGAATGGT | 4562 | 2384 | 2000 | 384 |
| 991 | IAGVAQS | 4563 | ATAGCTGGAGTGGCTCAATCA | 4564 | 2384 | 1960 | 424 |
| 992 | EYALTEA | 4565 | GAGTATGCGTTGACGGAGGCT | 4566 | 2383 | 0 | 2383 |
| 993 | AADSSGR | 4567 | GCCGCAGACAGCAGTGGCAGG | 4568 | 2383 | 0 | 2383 |
| 994 | ILVDAHT | 4569 | ATCCTAGTGGACGCGCACACA | 4570 | 2382 | 2000 | 382 |
| 995 | ADVHVRL | 4571 | GCTGATGTGCATGTGCGTTTG | 4572 | 2382 | 0 | 2382 |
| 996 | YVQAVPS | 4573 | TATGTGCAGGCGGTTCCTTCT | 4574 | 2381 | 2000 | 381 |
| 997 | ALAQNNM | 4575 | GCCTTAGCCCAAAACAACATG | 4576 | 2380 | 2000 | 380 |
| 998 | QGGDSGG | 4577 | CAAGGCGGAGACTCGGGTGGG | 4578 | 2379 | 2379 | 0 |
| 999 | RVEISAK | 4579 | AGGGTTGAGATTTCTGCGAAG | 4580 | 2379 | 1802 | 577 |
| 1000 | LGRVEHT | 4581 | TTAGGCAGAGTGGAACACACT | 4582 | 2379 | 0 | 2379 |

Macaque_allCNS_peptide rank

| | | SEQ | | SEQ | |
|---|---|---|---|---|---|
| | | ID | | ID | |
| Rank | Peptide | NO: | Sequence | NO: | k-medoids cluster # |
| 1 | PTQGTVR | 4583 | CCCACACAAGGCACAGTCCGT | 4584 | 128 |
| 2 | PTQGTFR | 4585 | CCGACACAAGGAACATTCAGG | 4586 | 128 |
| 3 | PSQGTLR | 4587 | CCTTCTCAGGGGACGCTTCGG | 4588 | 128 |
| 4 | NLGAALS | 4589 | AACCTTGGGGCTGCCCTATCG | 4590 | 203 |
| 5 | PKPSHGE | 4591 | CCTAAACCATCTCACGGAGAA | 4592 | 62 |
| 6 | PTPGTLR | 4593 | CCTACTCCGGGGACGCTTCGG | 4594 | 128 |
| 7 | PTQGTLR | 4595 | CCTACTCAGGGGACGCTTCGG | 4596 | 128 |
| 8 | QDGPAVK | 4597 | CAGGATGGGCCTGCGGTGAAG | 4598 | 260 |
| 9 | PNQGTLR | 4599 | CCAAACCAAGGTACTCTACGA | 4600 | 128 |
| 10 | ESLAGVR | 4601 | GAATCGTTGGCAGGGGTGCGT | 4602 | 316 |
| 11 | AADSSAR | 4603 | GCCGCTGACTCATCGGCCCGT | 4604 | 10 |
| 12 | SHGSDPK | 4605 | TCTCATGGTTCTGATCCGAAG | 4606 | 174 |
| 13 | AAGVIPN | 4607 | GCCGCCGGAGTGATACCTAAC | 4608 | 179 |
| 14 | KNPGVDT | 4609 | AAAAACCCTGGAGTTGACACG | 4610 | 337 |
| 15 | PAQGTLR | 4611 | CCGGCGCAAGGAACACTACGA | 4612 | 128 |
| 16 | GRSQLPM | 4613 | GGCCGATCACAACTTCCAATG | 4614 | 239 |
| 17 | VTTLSPV | 4615 | GTCACGACTTTGAGTCCAGTT | 4616 | 272 |
| 18 | HSEGVGR | 4617 | CACTCGGAAGGAGTCGGACGC | 4618 | 134 |
| 19 | SHGYDSK | 4619 | TCTCATGGTTATGATTCGAAG | 4620 | 174 |
| 20 | NQLGELV | 4621 | AACCAACTCGGCGAACTAGTG | 4622 | 209 |
| 21 | NGMGDVT | 4623 | AACGGCATGGGGGACGTTACT | 4624 | 312 |
| 22 | VGGNVVH | 4625 | GTTGGTGGTAATGTTGTTCAT | 4626 | 111 |
| 23 | LVTGMSS | 4627 | CTTGTTACTGGGATGAGTTCT | 4628 | 22 |
| 24 | MNVGHVL | 4629 | ATGAATGTGGGTCATGTTCTG | 4630 | 268 |
| 25 | MSISEPR | 4631 | ATGTCTATTAGTGAGCCGCGG | 4632 | 207 |
| 26 | ALGDALR | 4633 | GCACTAGGCGACGCATTACGC | 4634 | 191 |
| 27 | QYAVSGG | 4635 | CAATACGCAGTGAGCGGCGGT | 4636 | 345 |
| 28 | VLASLGP | 4637 | GTTCTGGCTTCGCTTGGTCCT | 4638 | 176 |
| 29 | GRDLTPA | 4639 | GGTCGGGATCTTACGCCTGCT | 4640 | 235 |
| 30 | RIVDSVP | 4641 | AGGATTGTGGATAGTGTTCCG | 4642 | 120 |
| 31 | VDHGGVV | 4643 | GTGGATCATGGTGGTGTGGTT | 4644 | 274 |
| 32 | ASDAVLR | 4645 | GCATCCGACGCCGTCCTAAGG | 4646 | 31 |
| 33 | ILVDAYA | 4647 | ATACTAGTAGACGCGTACGCT | 4648 | 175 |
| 34 | PTEGTLR | 4649 | CCGACAGAAGGCACACTGCGA | 4650 | 128 |
| 35 | TDALTTK | 4651 | ACTGATGCGCTTACGACTAAG | 4652 | 145 |
| 36 | RVDSEKL | 4653 | AGGGTGGATTCGGAGAAGCTT | 4654 | 146 |
| 37 | KNPGVDS | 4655 | AAGAATCCGGGGGTGGATTCT | 4656 | 337 |
| 38 | RTDGADH | 4657 | CGCACAGACGGAGCAGACCAC | 4658 | 252 |
| 39 | LSSTDGV | 4659 | CTGAGTTCGACTGATGGGGTT | 4660 | 151 |
| 40 | AVFSSQK | 4661 | GCTGTATTCTCCAGTCAAAAA | 4662 | 39 |
| 41 | TVITGAP | 4663 | ACTGTGATCACTGGCGCCCCC | 4664 | 44 |
| 42 | LESAAMI | 4665 | CTGGAGTCGGCTGCTATGATT | 4666 | 41 |
| 43 | RVLTSDV | 4667 | CGTGTTCTGACGTCTGATGTG | 4668 | 159 |
| 44 | PEPRSSY | 4669 | CCTGAGCCGCGTAGTAGTTAT | 4670 | 204 |
| 45 | ESRNDVV | 4671 | GAGTCGAGGAATGATGTTGTT | 4672 | 173 |
| 46 | RHIADAS | 4673 | AGACACATAGCGGACGCGTCG | 4674 | 347 |
| 47 | HAAGASS | 4675 | CATGCGGCGGGTGCTAGTAGT | 4676 | 46 |
| 48 | HTLSTGV | 4677 | CACACCCTAAGCACGGGAGTA | 4678 | 171 |
| 49 | TVADPRA | 4679 | ACTGTTGCGGATCCGCGGGCG | 4680 | 296 |
| 50 | SAGGSLQ | 4681 | AGTGCTGGTGGGAGTCTTCAG | 4682 | 49 |
| 51 | MANMLSV | 4683 | ATGGCGAACATGTTATCGGTG | 4684 | 167 |
| 52 | SLGEGRH | 4685 | AGTTTAGGCGAAGGGCGTCAC | 4686 | 90 |
| 53 | RESLEAL | 4687 | AGGGAGAGTCTTGAGGCGTTG | 4688 | 270 |
| 54 | LAGLGGP | 4689 | CTTGCGGGTTTGGGGGGGCCT | 4690 | 195 |
| 55 | LSLNDVV | 4691 | CTGAGTTTGAATGATGTGGTT | 4692 | 173 |
| 56 | ATDSSVR | 4693 | GCCACCGACAGCAGTGTCCGT | 4694 | 55 |
| 57 | STINTLM | 4695 | AGTACTATTAATACTCTGATG | 4696 | 56 |
| 58 | LSRDVAV | 4697 | TTGTCGAGGGATGTGGCGGTT | 4698 | 97 |
| 59 | QYVVSGA | 4699 | CAGTATGTTGTTAGTGGTGCG | 4700 | 345 |
| 60 | LIGAALD | 4701 | CTAATCGGCGCAGCACTCGAC | 4702 | 203 |
| 61 | TMANSER | 4703 | ACGATGGCAAACTCGGAACGC | 4704 | 60 |
| 62 | GINEHVA | 4705 | GGGATCAACGAACACGTAGCC | 4706 | 331 |
| 63 | SNLGETV | 4707 | TCGAATTTGGGGGAGACGGTT | 4708 | 180 |
| 64 | GARMVMT | 4709 | GGTGCGCGGATGGTTATGACT | 4710 | 269 |
| 65 | AMGGETA | 4711 | GCGATGGGTGGTGAGACTGCT | 4712 | 185 |
| 66 | PTHGTLR | 4713 | CCGACCCACGGTACACTGCGA | 4714 | 128 |
| 67 | LNGVTIT | 4715 | CTCAACGGCGTCACCATCACC | 4716 | 305 |
| 68 | SVSHVVV | 4717 | TCGGTCTCTCACGTCGTCGTA | 4718 | 202 |
| 69 | DVVLLTR | 4719 | GATGTTGTTTTGTTGACTAGG | 4720 | 6 |
| 70 | VGLLATV | 4721 | GTCGGTCTCCTTGCAACAGTG | 4722 | 336 |
| 71 | ASESSTR | 4723 | GCATCTGAAAGCTCAACACGG | 4724 | 70 |
| 72 | RVGSSED | 4725 | CGGGTTGGGAGCTCCGAAGAC | 4726 | 306 |
| 73 | LRVTENP | 4727 | CTTCGGGTCACCGAAAACCCC | 4728 | 237 |
| 74 | VTEHTQF | 4729 | GTGACTGAGCATACGCAGTTT | 4730 | 162 |
| 75 | SQAEGSV | 4731 | TCCCAAGCGGAAGGCAGCGTG | 4732 | 74 |
| 76 | VLLGINT | 4733 | GTCCTGCTCGGAATAAACACC | 4734 | 283 |
| 77 | LDSGIPR | 4735 | CTCGACTCTGGTATCCCCAGA | 4736 | 134 |
| 78 | GLGLAAN | 4737 | GGTTTGGGTTTGGCGGCGAAT | 4738 | 225 |
| 79 | VMSGTSH | 4739 | GTTATGTCGGGTACTAGTCAT | 4740 | 238 |
| 80 | DVAAGYR | 4741 | GACGTAGCGGCAGGATACCGA | 4742 | 143 |
| 81 | SIGDLGK | 4743 | AGTATCGGTGACCTAGGTAAA | 4744 | 137 |
| 82 | NGSSIGV | 4745 | AACGGCTCATCTATCGGCGTG | 4746 | 299 |
| 83 | LERGHMY | 4747 | CTCGAAAGAGGCCACATGTAC | 4748 | 41 |
| 84 | ITENASR | 4749 | ATTACTGAGAATGCGTCGCGG | 4750 | 83 |
| 85 | VHDSTPL | 4751 | GTGCATGATTCGACTCCGTTG | 4752 | 325 |
| 86 | TLALSER | 4753 | ACCTTAGCCTTATCAGAACGA | 4754 | 85 |
| 87 | TVDSPMR | 4755 | ACCGTCGACAGCCCTATGCGA | 4756 | 40 |
| 88 | STLHTSI | 4757 | AGTACTCTTCATACTTCGATT | 4758 | 254 |
| 89 | VGSLTAS | 4759 | GTGGGGTCGCTTACGGCTAGT | 4760 | 88 |
| 90 | MAGGTNP | 4761 | ATGGCAGGTGGCACAAACCCT | 4762 | 195 |
| 91 | SLSDGSL | 4763 | TCTCTGTCTGATGGTTCTCTT | 4764 | 90 |
| 92 | IHFSGDN | 4765 | ATCCACTTCAGCGGCGACAAC | 4766 | 45 |
| 93 | TGRVEAA | 4767 | ACGGGTAGGGTTGAGGCGGCG | 4768 | 333 |
| 94 | TTAAIVT | 4769 | ACGACGGCGGCTATTGTTACG | 4770 | 93 |
| 95 | SIQSEVT | 4771 | AGCATCCAATCCGAAGTTACC | 4772 | 4 |
| 96 | DSSGGGT | 4773 | GACAGCTCAGGCGGGGGCACA | 4774 | 37 |
| 97 | TMAISDR | 4775 | ACTATGGCGATTTCTGATCGG | 4776 | 262 |
| 98 | RVENGGT | 4777 | CGAGTGGAAAACGGCGGGACC | 4778 | 295 |
| 99 | REALALT | 4779 | AGGGAGGCGCTGGCTCTGACG | 4780 | 270 |
| 100 | IVTPTNT | 4781 | ATTGTTACTCCTACGAATACG | 4782 | 77 |
| 101 | LTSDNLA | 4783 | CTTACCTCAGACAACCTAGCC | 4784 | 280 |
| 102 | DAPRDGA | 4785 | GACGCACCCCGCGACGGGGCT | 4786 | 151 |
| 103 | AVLSQNI | 4787 | GCTGTGTTGTCTCAGAATATT | 4788 | 102 |
| 104 | VLLGSNR | 4789 | GTGCTTTTGGGTAGTAATAGG | 4790 | 283 |
| 105 | SMAVTAK | 4791 | AGTATGGCGGTGACGGCGAAG | 4792 | 104 |
| 106 | WSSELHA | 4793 | TGGTCTAGTGAGTTGCATGCT | 4794 | 33 |
| 107 | ENTVSPV | 4795 | GAAAACACAGTGAGCCCCGTC | 4796 | 272 |
| 108 | LNMGPLH | 4797 | CTGAATATGGGTCCTTTGCAT | 4798 | 231 |
| 109 | GRGTNDH | 4799 | GGTCGGGGTACGAATGATCAT | 4800 | 225 |
| 110 | STEYAML | 4801 | TCTACTGAGTATGCGATGTTG | 4802 | 8 |
| 111 | MGSNGQV | 4803 | ATGGGGTCTAATGGGCAGGTT | 4804 | 286 |
| 112 | LAGSVVV | 4805 | CTGGCGGGTTCGGTTGTTGTG | 4806 | 111 |
| 113 | YSMTVTT | 4807 | TATAGTATGACGGTTACGACT | 4808 | 80 |
| 114 | VLVGTSL | 4809 | GTTCTTGTTGGGACGAGTTTG | 4810 | 113 |
| 115 | MVTPTNR | 4811 | ATGGTGACACCCACAAACCGC | 4812 | 77 |
| 116 | VTTLTPV | 4813 | GTGACTACGCTTACTCCTGTG | 4814 | 272 |
| 117 | QTGEAAV | 4815 | CAGACGGGGGAGGCGGCGGTT | 4816 | 116 |
| 118 | NDRITST | 4817 | AACGACCGAATAACCTCAACT | 4818 | 157 |
| 119 | SDGKTHT | 4819 | TCAGACGGCAAAACCCACACC | 4820 | 33 |
| 120 | TSLLPQT | 4821 | ACGTCTCTGCTTCCTCAGACT | 4822 | 292 |
| 121 | PSLEHLA | 4823 | CCTAGTCTTGAGCATTTGGCT | 4824 | 349 |
| 122 | DHGSFAK | 4825 | GATCATGGTAGTTTTGCGAAG | 4826 | 174 |
| 123 | PTNGYPL | 4827 | CCCACAAACGGGTACCCGCTC | 4828 | 341 |
| 124 | TLTDVVH | 4829 | ACTCTTACTGATGTGGTGCAT | 4830 | 261 |
| 125 | LADGSVR | 4831 | TTAGCAGACGGCTCCGTCCGC | 4832 | 255 |
| 126 | GGVSSTN | 4833 | GGTGGTGTTTCTTCGACTAAT | 4834 | 335 |
| 127 | SHGTDSK | 4835 | AGTCACGGCACGGACTCTAAA | 4836 | 174 |
| 128 | ALATDMS | 4837 | GCGCTGGCTACTGATATGTCG | 4838 | 127 |
| 129 | QVRDTMT | 4839 | CAAGTCAGAGACACGATGACC | 4840 | 318 |
| 130 | NGYTEGR | 4841 | AATGGGTATACGGAGGGGCGT | 4842 | 299 |
| 131 | DSRVSGD | 4843 | GATAGTCGTGTGTCGGGGGAT | 4844 | 37 |
| 132 | VLSGEEL | 4845 | GTTCTTAGTGGGGAGGAGTTG | 4846 | 131 |
| 133 | HNGQVGV | 4847 | CATAATGGGCAGGTTGGTGTG | 4848 | 299 |
| 134 | HNSHVLT | 4849 | CACAACTCCCACGTATTAACC | 4850 | 0 |
| 135 | RPEIEVR | 4851 | CGGCCGGAGATTGAGGTTAGG | 4852 | 38 |
| 136 | KGSDSPM | 4853 | AAAGGATCGGACTCACCGATG | 4854 | 278 |
| 137 | DQLNDGR | 4855 | GATCAGCTGAATGATGGGCGG | 4856 | 54 |
| 138 | SLLHDGA | 4857 | AGTTTGTTGCATGATGGGGCG | 4858 | 34 |
| 139 | RDTQYDH | 4859 | CGTGATACGCAGTATGATCAT | 4860 | 261 |
| 140 | PREHNQA | 4861 | CCGCGTGAGCATAATCAGGCT | 4862 | 338 |
| 141 | SRLENIS | 4863 | TCGCGTCTTGAAAACATCTCC | 4864 | 349 |
| 142 | FDQTHKT | 4865 | TTTGATCAGACGCATAAGACT | 4866 | 245 |
| 143 | MTGISIV | 4867 | ATGACAGGCATCTCTATCGTA | 4868 | 142 |
| 144 | ASSHVTV | 4869 | GCTTCGAGTCATGTTACTGTG | 4870 | 0 |
| 145 | SHGSDLK | 4871 | AGCCACGGGAGCGACCTAAAA | 4872 | 174 |
| 146 | NIGADPK | 4873 | AACATCGGGGCCGACCCCAAA | 4874 | 54 |
| 147 | TLGSLSQ | 4875 | ACACTAGGGTCCCTGTCACAA | 4876 | 137 |
| 148 | PTQGTIR | 4877 | CCTACTCAGGGGACGATTCGG | 4878 | 128 |
| 149 | FTGGTGT | 4879 | TTTACTGGTGGTACGGGTACT | 4880 | 164 |
| 150 | IPSTGAQ | 4881 | ATTCCGAGTACGGGGGCGCAG | 4882 | 30 |
| 151 | STLHTTT | 4883 | AGTACTCTTCATACTACGACT | 4884 | 150 |
| 152 | GGTNSAH | 4885 | GGTGGAACAAACTCAGCGCAC | 4886 | 335 |
| 153 | NVGLVSP | 4887 | AACGTAGGGCTCGTATCACCA | 4888 | 197 |
| 154 | VYESTVR | 4889 | GTTTATGAGAGTACGGTGAGG | 4890 | 153 |
| 155 | MGASDTH | 4891 | ATGGGGGCTAGTGATACGCAT | 4892 | 26 |
| 156 | VIATGNP | 4893 | GTTATTGCTACGGGGAATCCT | 4894 | 176 |
| 157 | PEQQKVW | 4895 | CCTGAGCAGCAGAAGGTTTGG | 4896 | 94 |
| 158 | SDGQFGR | 4897 | TCTGACGGTCAATTCGGACGA | 4898 | 54 |
| 159 | IMTSVTM | 4899 | ATCATGACAAGTGTTACAATG | 4900 | 136 |
| 160 | AQDHGTL | 4901 | GCGCAGGATCATGGGACGTTG | 4902 | 286 |
| 161 | GGLVVVG | 4903 | GGCGGACTAGTAGTCGTGGGG | 4904 | 269 |
| 162 | TSVESNL | 4905 | ACGTCGGTGGAGTCGAATCTT | 4906 | 135 |
| 163 | SVTDIKH | 4907 | TCGGTGACGGACATAAAACAC | 4908 | 261 |
| 164 | LSMTDGL | 4909 | CTGAGTATGACTGATGGGCTT | 4910 | 151 |
| 165 | LNMKADG | 4911 | TTAAACATGAAAGCAGACGGA | 4912 | 192 |
| 166 | LNSGVSR | 4913 | CTCAACAGTGGTGTCAGCCGC | 4914 | 134 |
| 167 | STIPTLL | 4915 | AGTACTATTCCTACTCTGTTG | 4916 | 166 |
| 168 | TFGIDAS | 4917 | ACATTCGGAATCGACGCGTCC | 4918 | 5 |
| 169 | AVGVILN | 4919 | GCGGTGGGTGTTATTCTGAAT | 4920 | 179 |
| 170 | GSREDVR | 4921 | GGGAGTAGGGAGGATGTGCGT | 4922 | 343 |
| 171 | HLHNTLN | 4923 | CATCTTCATAATACTCTTAAT | 4924 | 56 |
| 172 | VSVAVGL | 4925 | GTTTCGGTGGCTGTTGGGTTG | 4926 | 133 |
| 173 | LGVSRDL | 4927 | CTGGGTGTGTCTCGGGATCTG | 4928 | 99 |
| 174 | KGSDNTM | 4929 | AAGGGTTCTGATAATACTATG | 4930 | 278 |
| 175 | ASIPTLN | 4931 | GCATCCATACCAACGCTAAAC | 4932 | 332 |
| 176 | YHASDSK | 4933 | TATCATGCTTCTGATTCGAAG | 4934 | 174 |
| 177 | QYSELHH | 4935 | CAATACTCCGAATTGCACCAC | 4936 | 261 |
| 178 | DLTTPVR | 4937 | GATTTGACTACTCCGGTGCGT | 4938 | 342 |
| 179 | IGTEISS | 4939 | ATTGGTACGGAGATTTCGTCG | 4940 | 184 |
| 180 | STDMRSP | 4941 | AGTACGGATATGAGGTCGCCG | 4942 | 319 |
| 181 | TKITNED | 4943 | ACAAAAATCACTAACGAAGAC | 4944 | 258 |
| 182 | STLQGEA | 4945 | AGCACCCTCCAAGGGGAAGCA | 4946 | 181 |
| 183 | PLLGNTI | 4947 | CCGCTTTTGGGGAATACGATT | 4948 | 328 |
| 184 | NGLQVSI | 4949 | AATGGGCTGCAGGTTAGTATT | 4950 | 190 |
| 185 | AVTNPLM | 4951 | GCGGTTACTAATCCTTTGATG | 4952 | 208 |
| 186 | DVTVSMR | 4953 | GATGTTACTGTTTCTATGCGT | 4954 | 342 |
| 187 | NQLAEQV | 4955 | AATCAGTTGGCGGAGCAGGTT | 4956 | 226 |
| 188 | RPDASST | 4957 | AGGCCTGATGCTTCTTCGACG | 4958 | 311 |
| 189 | DTSLRLM | 4959 | GACACCTCTCTACGCCTTATG | 4960 | 35 |
| 190 | TLPELKL | 4961 | ACGTTGCCGGAGTTGAAGCTT | 4962 | 301 |
| 191 | QNGLQLL | 4963 | CAGAATGGGTTGCAGCTTTTG | 4964 | 142 |
| 192 | ASREVLY | 4965 | GCCAGTCGCGAAGTACTCTAC | 4966 | 343 |
| 193 | IASDIGR | 4967 | ATTGCTTCGGATATTGGTCGG | 4968 | 309 |
| 194 | VADSYNL | 4969 | GTCGCAGACAGTTACAACCTA | 4970 | 141 |
| 195 | STVGINV | 4971 | AGTACGGTCGGGATCAACGTT | 4972 | 194 |
| 196 | GVAGRIL | 4973 | GGGGTGGCTGGGCGTATTCTG | 4974 | 149 |
| 197 | NEAVNVR | 4975 | AATGAGGCTGTTAATGTTCGG | 4976 | 91 |
| 198 | TVGHDNK | 4977 | ACCGTAGGACACGACAACAAA | 4978 | 54 |
| 199 | TLQQLQL | 4979 | ACTCTCCAACAACTGCAATTG | 4980 | 301 |
| 200 | ALSGLAN | 4981 | GCATTGAGCGGCCTGGCGAAC | 4982 | 199 |
| 201 | ALGTQGS | 4983 | GCTCTGGGTACGCAGGGTTCT | 4984 | 291 |
| 202 | PNERLAV | 4985 | CCTAACGAACGATTGGCAGTC | 4986 | 144 |
| 203 | GVAATNT | 4987 | GGAGTTGCAGCCACAAACACG | 4988 | 132 |
| 204 | WDHNSLK | 4989 | TGGGACCACAACAGCTTGAAA | 4990 | 61 |
| 205 | LVGVVEP | 4991 | CTTGTTGGTGTGGTTGAGCCG | 4992 | 287 |
| 206 | TLTDRAS | 4993 | ACGTTGACGGATAGGGCGTCT | 4994 | 205 |
| 207 | SGSNTGH | 4995 | TCGGGGTCTAATACGGGTCAT | 4996 | 299 |
| 208 | VLASHGT | 4997 | GTTCTGGCTTCGCATGGTACT | 4998 | 176 |
| 209 | AVGNVLL | 4999 | GCTGTGGGGAATGTGCTTTTG | 5000 | 208 |
| 210 | NTVVNDP | 5001 | AACACAGTCGTGAACGACCCT | 5002 | 120 |
| 211 | KLMDSRD | 5003 | AAGCTGATGGATTCGCGGGAT | 5004 | 249 |
| 212 | RNQPEAM | 5005 | AGGAACCAACCAGAAGCCATG | 5006 | 252 |
| 213 | VIAGLGV | 5007 | GTGATCGCGGGACTCGGCGTC | 5008 | 212 |
| 214 | RGQSDPL | 5009 | CGGGGGCAGTCTGATCCGTTG | 5010 | 26 |
| 215 | GLNEHES | 5011 | GGGCTTAACGAACACGAATCT | 5012 | 331 |
| 216 | GVVNDER | 5013 | GGCGTTGTCAACGACGAACGG | 5014 | 60 |
| 217 | GTVGSMV | 5015 | GGTACGGTGGGTTCTATGGTT | 5016 | 216 |
| 218 | LTGERIL | 5017 | CTAACCGGTGAACGCATACTT | 5018 | 142 |
| 219 | PTQGVSM | 5019 | CCAACCCAAGGAGTTTCGATG | 5020 | 128 |
| 220 | AAREELN | 5021 | GCGGCTCGGGAGGAGCTTAAT | 5022 | 343 |
| 221 | FNGLPAQ | 5023 | TTCAACGGTCTCCCCGCACAA | 5024 | 160 |
| 222 | HTIAASM | 5025 | CACACCATAGCCGCAAGTATG | 5026 | 221 |
| 223 | TDAGDGK | 5027 | ACAGACGCGGGGGACGGCAAA | 5028 | 157 |
| 224 | SDLRPPL | 5029 | TCGGATCTTCGGCCGCCGCTG | 5030 | 244 |
| 225 | AGLSQNL | 5031 | GCTGGGTTGTCTCAGAATCTT | 5032 | 224 |
| 226 | GMGASSK | 5033 | GGTATGGGGGCGTCTTCTAAG | 5034 | 225 |
| 227 | SQLAELV | 5035 | AGTCAGTTGGCGGAGCTGGTT | 5036 | 209 |
| 228 | LTRGEEK | 5037 | CTTACGAGGGGTGAGGAGAAG | 5038 | 294 |
| 229 | AGGVILN | 5039 | GCGGGGGGTGTTATTCTGAAT | 5040 | 179 |
| 230 | GNGTGVL | 5041 | GGAAACGGCACCGGGGTCCTA | 5042 | 172 |
| 231 | VVSGIPN | 5043 | GTGGTGTCTGGTATTCCGAAT | 5044 | 199 |
| 232 | GVMAAGI | 5045 | GGAGTCATGGCCGCGGGTATC | 5046 | 34 |
| 233 | GVANESP | 5047 | GGTGTGGCGAATGAGAGTCCG | 5048 | 197 |
| 234 | ELMASTI | 5049 | GAGCTTATGGCTTCTACTATT | 5050 | 130 |
| 235 | NLGVVQV | 5051 | AACCTAGGAGTCGTACAAGTC | 5052 | 28 |
| 236 | RTTPDVP | 5053 | CGTACGACTCCCGACGTACCT | 5054 | 107 |
| 237 | LESLSHH | 5055 | CTGGAGTCGCTTTCTCATCAT | 5056 | 41 |
| 238 | LSLTHGD | 5057 | CTGAGTTTGACTCATGGGGAT | 5058 | 259 |
| 239 | DGVNTAL | 5059 | GATGGGGTTAATACGGCGTTG | 5060 | 154 |
| 240 | QDGPAEK | 5061 | CAGGATGGGCCTGCGGAGAAG | 5062 | 260 |
| 241 | SPAGLGK | 5063 | AGCCCCGCGGGCCTAGGCAAA | 5064 | 12 |
| 242 | RYNDEST | 5065 | AGATACAACGACGAATCCACT | 5066 | 295 |
| 243 | VVAGTNS | 5067 | GTCGTTGCAGGTACAAACTCG | 5068 | 108 |
| 244 | WSGQIHV | 5069 | TGGAGTGGTCAGATTCATGTG | 5070 | 264 |
| 245 | ANSHTNS | 5071 | GCAAACAGTCACACCAACTCT | 5072 | 303 |
| 246 | VVQAPGR | 5073 | GTTGTTCAGGCTCCTGGGCGT | 5074 | 176 |
| 247 | ATQGTLR | 5075 | GCTACTCAGGGGACGCTTCGG | 5076 | 128 |
| 248 | RVDPSGL | 5077 | CGTGTGGATCCTTCTGGGCTG | 5078 | 86 |
| 249 | TKDIGVM | 5079 | ACGAAAGACATAGGCGTAATG | 5080 | 200 |
| 250 | GGKGEGP | 5081 | GGTGGGAAGGGTGAGGGTCCG | 5082 | 149 |
| 251 | RGAVSTE | 5083 | CGGGGGGCTGTGTCGACTGAG | 5084 | 250 |
| 252 | STDRESR | 5085 | TCGACTGATCGGGAGTCGCGG | 5086 | 72 |
| 253 | NLHTAEA | 5087 | AACCTCCACACTGCTGAAGCG | 5088 | 302 |
| 254 | MSTAMSL | 5089 | ATGTCGACGGCGATGAGTCTG | 5090 | 126 |
| 255 | TGSSAML | 5091 | ACGGGGAGTTCGGCGATGCTT | 5092 | 125 |
| 256 | LISGTLR | 5093 | TTGATTTCTGGTACGCTGCGT | 5094 | 124 |
| 257 | GIGGVIS | 5095 | GGTATTGGTGGTGTGATTTCG | 5096 | 234 |
| 258 | RLENRGV | 5097 | CGGTTGGAGAATAGGGGGGTT | 5098 | 243 |
| 259 | LPNGGGF | 5099 | CTGCCGAATGGGGGGGGGTTT | 5100 | 30 |
| 260 | TGDRDQN | 5101 | ACTGGTGATCGGGATCAGAAT | 5102 | 251 |
| 261 | SLAITER | 5103 | AGTTTGGCGATTACTGAGCGG | 5104 | 123 |
| 262 | STLISET | 5105 | TCCACGTTGATATCAGAAACC | 5106 | 122 |
| 263 | DVRGSDI | 5107 | GACGTACGGGGGTCTGACATC | 5108 | 156 |
| 264 | NLSLSLR | 5109 | AATCTGTCTCTGTCGTTGCGT | 5110 | 263 |
| 265 | GSGGVSV | 5111 | GGTTCGGGTGGTGTTAGTGTG | 5112 | 264 |
| 266 | LVSGLGP | 5113 | CTTGTGAGTGGGCTGGGTCCG | 5114 | 212 |
| 267 | LTKSTEW | 5115 | CTCACCAAATCCACAGAATGG | 5116 | 281 |
| 268 | TTRADPA | 5117 | ACTACTCGGGCTGATCCTGCG | 5118 | 273 |
| 269 | ASMSAVN | 5119 | GCATCAATGTCAGCTGTCAAC | 5120 | 121 |
| 270 | RVDSAQP | 5121 | AGAGTAGACAGTGCCCAACCC | 5122 | 186 |
| 271 | EPSLGSK | 5123 | GAACCAAGTCTCGGGTCGAAA | 5124 | 157 |
| 272 | YLGADAA | 5125 | TATCTTGGTGCTGATGCTGCT | 5126 | 68 |
| 273 | RDEAYRA | 5127 | AGGGATGAGGCTTATCGTGCG | 5128 | 233 |
| 274 | RVAMSVT | 5129 | AGGGTGGCGATGTCTGTGACG | 5130 | 32 |
| 275 | SHGSDSN | 5131 | TCGCACGGCTCCGACTCCAAC | 5132 | 174 |
| 276 | ETRMISE | 5133 | GAGACGCGTATGATTTCGGAG | 5134 | 167 |
| 277 | AHIGTLT | 5135 | GCACACATCGGAACTCTCACC | 5136 | 53 |
| 278 | MGGVTNP | 5137 | ATGGGAGGTGTCACCAACCCC | 5138 | 195 |
| 279 | PSPSVTL | 5139 | CCGTCGCCTAGTGTTACTTTG | 5140 | 95 |
| 280 | SSSGAAW | 5141 | TCGAGTAGTGGGGCGGCGTGG | 5142 | 8 |
| 281 | HTQGTLR | 5143 | CATACTCAGGGGACGCTTCGG | 5144 | 128 |
| 282 | LTDVTQM | 5145 | TTAACCGACGTCACACAAATG | 5146 | 281 |
| 283 | ILSSATD | 5147 | ATTCTTAGTTCGGCGACTGAT | 5148 | 119 |
| 284 | NGSNDLS | 5149 | AACGGTAGCAACGACCTTTCA | 5150 | 59 |
| 285 | KGSDNHM | 5151 | AAAGGCAGTGACAACCACATG | 5152 | 278 |
| 286 | QEQGTTT | 5153 | CAGGAGCAGGGTACGACTACT | 5154 | 285 |
| 287 | PGVAMVT | 5155 | CCCGGGGTCGCTATGGTAACT | 5156 | 17 |
| 288 | LVGVSSE | 5157 | CTGGTGGGTGTGTCGTCTGAG | 5158 | 287 |
| 289 | SGGTRGP | 5159 | TCTGGTGGGACTCGTGGTCCT | 5160 | 310 |
| 290 | AIQTNDA | 5161 | GCAATCCAAACCAACGACGCG | 5162 | 289 |
| 291 | LISTTLR | 5163 | TTGATTTCTACTACGCTGCGT | 5164 | 118 |
| 292 | ALGDQAR | 5165 | GCGTTAGGGGACCAAGCGCGT | 5166 | 291 |
| 293 | GLNDHVA | 5167 | GGTCTGAATGATCATGTGGCG | 5168 | 331 |
| 294 | NDVSLAT | 5169 | AATGATGTGAGTCTGGCTACT | 5170 | 293 |
| 295 | KMAITDD | 5171 | AAAATGGCTATAACAGACGAC | 5172 | 147 |
| 296 | LSNHGPI | 5173 | CTGAGTAATCATGGGCCTATT | 5174 | 286 |
| 297 | VLNDNLA | 5175 | GTGTTAAACGACAACTTAGCT | 5176 | 280 |
| 298 | RHVHVEG | 5177 | CGCCACGTACACGTCGAAGGC | 5178 | 236 |
| 299 | DGRAELR | 5179 | GATGGGCGGGCGGAGTTGCGT | 5180 | 333 |
| 300 | SGISFLA | 5181 | AGCGGAATCAGCTTCTTGGCT | 5182 | 154 |
| 301 | RISPEGT | 5183 | CGTATATCACCGGAAGGCACT | 5184 | 295 |
| 302 | RVTPTNT | 5185 | CGCGTGACGCCAACTAACACT | 5186 | 77 |
| 303 | STTSSPS | 5187 | TCGACCACCTCATCCCCTAGC | 5188 | 117 |
| 304 | LLHGIIA | 5189 | CTTTTACACGGAATAATCGCC | 5190 | 298 |
| 305 | ASESSPP | 5191 | GCATCAGAATCATCACCACCC | 5192 | 139 |
| 306 | TSREEQW | 5193 | ACTTCTCGTGAGGAGCAGTGG | 5194 | 343 |
| 307 | AGDRDQY | 5195 | GCTGGTGATCGGGATCAGTAT | 5196 | 251 |
| 308 | GVAIALQ | 5197 | GGGGTTGCTATTGCTCTTCAG | 5198 | 307 |
| 309 | LLGGTLA | 5199 | TTGTTGGGGGGTACTCTGGCT | 5200 | 298 |
| 310 | ALKEYES | 5201 | GCGCTGAAGGAGTATGAGTCG | 5202 | 277 |
| 311 | RGGKEEM | 5203 | CGAGGTGGCAAAGAAGAAATG | 5204 | 310 |
| 312 | VDFGDHT | 5205 | GTAGACTTCGGCGACCACACC | 5206 | 312 |
| 313 | GADVNNH | 5207 | GGTGCTGACGTCAACAACCAC | 5208 | 62 |
| 314 | MNGGNVL | 5209 | ATGAACGGCGGCAACGTGCTC | 5210 | 268 |
| 315 | HQGDTIV | 5211 | CATCAGGGGGATACGATTGTG | 5212 | 275 |
| 316 | SHGSDSR | 5213 | AGCCACGGGTCGGACTCCCGG | 5214 | 174 |
| 317 | TGHEGGS | 5215 | ACAGGCCACGAAGGAGGTTCG | 5216 | 327 |
| 318 | PNERHTL | 5217 | CCTAACGAACGCCACACCTTG | 5218 | 144 |
| 319 | TDALLMH | 5219 | ACAGACGCACTCCTCATGCAC | 5220 | 157 |
| 320 | SVTERSG | 5221 | TCTGTGACGGAGAGGAGTGGT | 5222 | 319 |
| 321 | LGHANGL | 5223 | TTAGGGCACGCAAACGGACTT | 5224 | 171 |
| 322 | GVSDFQS | 5225 | GGGGTATCGGACTTCCAATCA | 5226 | 330 |
| 323 | GVANVSP | 5227 | GGAGTTGCTAACGTCAGCCCA | 5228 | 197 |
| 324 | ATLLPQT | 5229 | GCCACACTTCTGCCACAAACG | 5230 | 122 |
| 325 | SSLLTTA | 5231 | TCGTCGTTGCTGACTACTGCT | 5232 | 115 |
| 326 | LNGAPLL | 5233 | CTGAATGGTGCGCCGTTGCTG | 5234 | 268 |
| 327 | SGSIVVV | 5235 | AGTGGTTCGATTGTGGTGGTT | 5236 | 114 |
| 328 | DVAISMR | 5237 | GACGTAGCGATATCCATGCGA | 5238 | 297 |
| 329 | LLADERV | 5239 | TTACTCGCAGACGAAAGGGTC | 5240 | 296 |
| 330 | LTSGLAA | 5241 | TTGACGTCTGGTTTGGCGGCG | 5242 | 112 |
| 331 | GPLNQSL | 5243 | GGTCCGCTGAATCAGTCTTTG | 5244 | 224 |
| 332 | EGSEHVK | 5245 | GAAGGGTCCGAACACGTGAAA | 5246 | 35 |
| 333 | RQDNSDV | 5247 | CGGCAGGATAATTCGGATGTG | 5248 | 159 |
| 334 | LGAGSLS | 5249 | TTGGGGGCGGGGAGTCTGTCT | 5250 | 110 |
| 335 | KLAEGVR | 5251 | AAACTAGCCGAAGGAGTGCGG | 5252 | 44 |
| 336 | HGTLESQ | 5253 | CACGGCACCCTCGAATCGCAA | 5254 | 178 |
| 337 | LDTSDRL | 5255 | TTGGACACGTCTGACCGGCTC | 5256 | 248 |
| 338 | VRGEETV | 5257 | GTTCGTGGGGAAGAAACCGTC | 5258 | 185 |
| 339 | VVLSLAT | 5259 | GTTGTCTTAAGTCTAGCCACT | 5260 | 109 |
| 340 | RDDQGIP | 5261 | CGGGATGATCAGGGGATTCCG | 5262 | 163 |
| 341 | AVAGTNS | 5263 | GCAGTTGCGGGTACAAACTCG | 5264 | 108 |
| 342 | RGGVTTE | 5265 | CGTGGAGGCGTAACCACCGAA | 5266 | 250 |
| 343 | RMTLTGD | 5267 | CGTATGACTTTGACTGGTGAT | 5268 | 247 |
| 344 | DDAVSKR | 5269 | GATGATGCTGTTTCTAAGCGT | 5270 | 143 |
| 345 | VNHGGVD | 5271 | GTAAACCACGGAGGAGTTGAC | 5272 | 274 |
| 346 | PGEPLRL | 5273 | CCGGGAGAACCCTTGCGACTC | 5274 | 279 |
| 347 | PGEHYEA | 5275 | CCGGGTGAGCATTATGAGGCT | 5276 | 196 |
| 348 | PSQGMTR | 5277 | CCTAGTCAGGGTATGACTCGT | 5278 | 128 |
| 349 | RASADVV | 5279 | AGGGCGAGTGCGGATGTTGTG | 5280 | 209 |
| 350 | DAQSRLA | 5281 | GATGCTCAGTCGCGGTTGGCG | 5282 | 290 |
| 351 | DPSLGSP | 5283 | GATCCGTCTCTGGGTTCTCCG | 5284 | 37 |
| 352 | RNVSDMT | 5285 | CGAAACGTGTCGGACATGACC | 5286 | 347 |
| 353 | VQSADPR | 5287 | GTCCAATCCGCGGACCCTCGC | 5288 | 27 |
| 354 | GVSTLSL | 5289 | GGGGTTTCTACTCTGAGTCTT | 5290 | 106 |
| 355 | REQQKAW | 5291 | CGAGAACAACAAAAAGCCTGG | 5292 | 169 |
| 356 | DTASTQS | 5293 | GACACAGCATCTACTCAATCC | 5294 | 105 |
| 357 | MSDSGTV | 5295 | ATGAGCGACTCGGGCACGGTT | 5296 | 286 |
| 358 | DSRTVDS | 5297 | GACTCTCGAACCGTCGACTCA | 5298 | 105 |
| 359 | NSGPQLS | 5299 | AACTCGGGCCCACAACTTTCG | 5300 | 203 |
| 360 | QKDSLVA | 5301 | CAGAAGGATTCGTTGGTTGCT | 5302 | 284 |
| 361 | TLATQEL | 5303 | ACTCTGGCGACGCAGGAGCTG | 5304 | 103 |
| 362 | QSMTDGV | 5305 | CAGAGTATGACTGATGGGGTT | 5306 | 151 |
| 363 | VTVAGSV | 5307 | GTTACGGTGGCTGGTTCGGTG | 5308 | 101 |
| 364 | SVVGLDS | 5309 | TCAGTCGTCGGATTAGACTCG | 5310 | 282 |
| 365 | MNGGHLM | 5311 | ATGAATGGGGGTCATCTTATG | 5312 | 268 |
| 366 | DKVVDEV | 5313 | GATAAGGTGGTTGATGAGGTG | 5314 | 226 |
| 367 | VVGTQDR | 5315 | GTTGTGGGGACTCAGGATAGG | 5316 | 211 |
| 368 | DVAVYIR | 5317 | GATGTTGCTGTTTATATTCGT | 5318 | 143 |
| 369 | EPSLGSR | 5319 | GAGCCGTCTCTGGGTTCTCGG | 5320 | 182 |
| 370 | ASVSALL | 5321 | GCGAGTGTTTCTGCGTTGTTG | 5322 | 100 |
| 371 | LSLDRPS | 5323 | CTAAGTCTAGACCGACCCTCG | 5324 | 327 |
| 372 | PAIQGNF | 5325 | CCAGCCATCCAAGGAAACTTC | 5326 | 135 |
| 373 | SVEPLSL | 5327 | TCCGTAGAACCTCTATCCCTC | 5328 | 279 |
| 374 | KSSDTPM | 5329 | AAGAGTTCTGATACTCCTATG | 5330 | 278 |
| 375 | SFDTYGA | 5331 | TCCTTCGACACTTACGGGGCC | 5332 | 62 |
| 376 | AVSDYAV | 5333 | GCAGTATCTGACTACGCAGTC | 5334 | 330 |
| 377 | AGVSASL | 5335 | GCGGGTGTTTCTGCGTCGTTG | 5336 | 99 |
| 378 | VVSQLPV | 5337 | GTCGTCTCTCAACTACCGGTA | 5338 | 276 |
| 379 | EIVLTVP | 5339 | GAGATTGTTTTGACTGTGCCG | 5340 | 120 |
| 380 | MIGGHVQ | 5341 | ATGATTGGGGGTCATGTTCAG | 5342 | 268 |
| 381 | TGLGLMV | 5343 | ACCGGACTCGGACTAATGGTA | 5344 | 323 |
| 382 | VTTHSPV | 5345 | GTTACCACCCACAGTCCAGTT | 5346 | 67 |
| 383 | VLPHANT | 5347 | GTCCTACCACACGCCAACACA | 5348 | 87 |
| 384 | STVLVPK | 5349 | TCTACCGTACTAGTCCCTAAA | 5350 | 244 |
| 385 | GGDALNQ | 5351 | GGGGGGGACGCCCTTAACCAA | 5352 | 31 |
| 386 | QIHDTAL | 5353 | CAAATCCACGACACAGCGCTC | 5354 | 318 |
| 387 | ALTNGQR | 5355 | GCACTAACCAACGGTCAACGT | 5356 | 60 |
| 388 | PARYRLW | 5357 | CCGGCGCGGTATCGGCTTTGG | 5358 | 35 |
| 389 | RNEGINQ | 5359 | CGTAATGAGGGTATTAATCAG | 5360 | 252 |
| 390 | QRSDSVM | 5361 | CAGCGGTCGGATAGTGTGATG | 5362 | 275 |
| 391 | NRQENSY | 5363 | AATCGGCAGGAGAATTCGTAT | 5364 | 349 |
| 392 | NDRNTSS | 5365 | AATGATAGGAATACGTCTTCG | 5366 | 242 |
| 393 | MNGGHVL | 5367 | ATGAATGGGGGTCATGTTCTG | 5368 | 268 |
| 394 | IPATADK | 5369 | ATCCCAGCCACGGCGGACAAA | 5370 | 140 |
| 395 | YAGIAQG | 5371 | TATGCGGGGATTGCTCAGGGT | 5372 | 269 |
| 396 | STQGGLA | 5373 | AGTACCCAAGGCGGATTAGCG | 5374 | 274 |
| 397 | GLLKNLD | 5375 | GGTTTGCTAAAAAACCTCGAC | 5376 | 349 |
| 398 | GLVQMSS | 5377 | GGTCTGGTGCAGATGTCTTCT | 5378 | 326 |
| 399 | GVSVPNV | 5379 | GGGGTGAGTGTGCCGAATGTT | 5380 | 132 |
| 400 | TTSRPEE | 5381 | ACTACTTCTCGGCCGGAGGAG | 5382 | 292 |
| 401 | RDMGALV | 5383 | CGGGATATGGGTGCTCTTGTG | 5384 | 231 |
| 402 | VHASSPT | 5385 | GTGCATGCTTCTAGTCCGACT | 5386 | 325 |
| 403 | REQQKYW | 5387 | CGGGAACAACAAAAATACTGG | 5388 | 169 |
| 404 | CNAAGCP | 5389 | TGTAATGCTGCGGGGTGTCCG | 5390 | 17 |
| 405 | PMRPGVA | 5391 | CCGATGCGGCCGGGTGTGGCT | 5392 | 200 |
| 406 | FGGVINA | 5393 | TTCGGGGGAGTAATAAACGCT | 5394 | 195 |
| 407 | STFSTVM | 5395 | AGCACATTCTCCACTGTTATG | 5396 | 98 |
| 408 | GHQNGGI | 5397 | GGGCACCAAAACGGCGGAATC | 5398 | 265 |
| 409 | MTSGNLM | 5399 | ATGACCTCTGGCAACCTCATG | 5400 | 280 |
| 410 | RESANAD | 5401 | CGTGAGTCTGCGAATGCTGAT | 5402 | 233 |
| 411 | SGDVARH | 5403 | TCAGGCGACGTTGCCCGACAC | 5404 | 17 |
| 412 | VSANVTI | 5405 | GTTTCTGCGAATGTTACGATT | 5406 | 97 |
| 413 | VPGSTTT | 5407 | GTTCCAGGCTCAACGACTACC | 5408 | 267 |
| 414 | PLVPQGG | 5409 | CCCTTAGTACCTCAAGGCGGT | 5410 | 34 |
| 415 | PGDRDQY | 5411 | CCAGGCGACCGAGACCAATAC | 5412 | 251 |
| 416 | HVSGASL | 5413 | CACGTGTCCGGCGCCAGCTTA | 5414 | 266 |
| 417 | YTSGTGT | 5415 | TACACCTCGGGCACAGGGACA | 5416 | 29 |
| 418 | PNTRDPI | 5417 | CCTAATACGCGGGATCCGATT | 5418 | 144 |
| 419 | SPVGIIA | 5419 | TCTCCTGTGGGTATTATTGCG | 5420 | 7 |
| 420 | LGDSDET | 5421 | TTAGGAGACTCGGACGAAACC | 5422 | 219 |
| 421 | NRHETLS | 5423 | AACCGCCACGAAACACTATCA | 5424 | 349 |
| 422 | GSVSSTK | 5425 | GGCTCCGTCAGTTCTACGAAA | 5426 | 96 |
| 423 | VFTGTDP | 5427 | GTGTTCACCGGCACAGACCCT | 5428 | 129 |
| 424 | YGSNVLS | 5429 | TACGGTTCTAACGTCCTCTCA | 5430 | 110 |
| 425 | TDNGALS | 5431 | ACTGATAATGGTGCGTTGTCG | 5432 | 110 |
| 426 | TGLGDRA | 5433 | ACCGGCTTGGGAGACAGGGCT | 5434 | 273 |
| 427 | DPSLGYP | 5435 | GACCCCAGTTTGGGCTACCCT | 5436 | 37 |
| 428 | LSLTEGV | 5437 | CTGAGTTTGACTGAGGGGGTT | 5438 | 259 |
| 429 | ARVLEKT | 5439 | GCCCGAGTCCTTGAAAAAACC | 5440 | 122 |
| 430 | VDTSARD | 5441 | GTTGATACTAGTGCTCGTGAT | 5442 | 248 |
| 431 | PTQETLR | 5443 | CCTACTCAGGAGACGCTTCGG | 5444 | 128 |
| 432 | AALTREI | 5445 | GCTGCTCTTACGCGGGAGATT | 5446 | 258 |
| 433 | RDLTNDV | 5447 | CGCGACTTAACTAACGACGTT | 5448 | 159 |
| 434 | GLSERAQ | 5449 | GGCCTGTCCGAACGAGCACAA | 5450 | 205 |
| 435 | DSLLPQT | 5451 | GATTCTCTGCTTCCTCAGACT | 5452 | 292 |
| 436 | LEANVSH | 5453 | CTTGAGGCGAATGTTTCGCAT | 5454 | 19 |
| 437 | AGSTVTW | 5455 | GCGGGGTCGACTGTTACTTGG | 5456 | 114 |
| 438 | YGVTLST | 5457 | TACGGCGTAACCCTCTCTACC | 5458 | 13 |
| 439 | GPSGAGI | 5459 | GGGCCATCAGGGGCAGGCATC | 5460 | 30 |
| 440 | VSNGHFV | 5461 | GTTAGTAATGGGCATTTTGTT | 5462 | 268 |
| 441 | GVSLPMS | 5463 | GGCGTATCACTACCCATGAGC | 5464 | 307 |
| 442 | MAASVTL | 5465 | ATGGCGGCTAGTGTTACGCTT | 5466 | 95 |
| 443 | KIGENAS | 5467 | AAGATTGGTGAGAATGCTTCT | 5468 | 321 |
| 444 | ISMTLLP | 5469 | ATTTCGATGACTCTGCTGCCG | 5470 | 184 |
| 445 | GAVSSTK | 5471 | GGTGCTGTTTCTTCGACTAAG | 5472 | 92 |
| 446 | TTLAHPA | 5473 | ACTACTCTGGCTCATCCTGCG | 5474 | 244 |
| 447 | LMNDLLS | 5475 | CTTATGAACGACTTACTCTCC | 5476 | 175 |
| 448 | TTAANVR | 5477 | ACGACGGCGGCTAATGTTAGG | 5478 | 91 |
| 449 | PNDRLTV | 5479 | CCAAACGACCGGTTGACGGTT | 5480 | 144 |
| 450 | LQVEQVM | 5481 | CTTCAGGTTGAGCAGGTTATG | 5482 | 329 |
| 451 | MLMGAET | 5483 | ATGCTCATGGGGGCAGAAACT | 5484 | 257 |
| 452 | LSLTMPA | 5485 | CTCTCGCTTACAATGCCTGCC | 5486 | 207 |
| 453 | KEIHVSV | 5487 | AAGGAGATTCATGTGTCGGTT | 5488 | 69 |
| 454 | MAVDVTK | 5489 | ATGGCAGTCGACGTAACCAAA | 5490 | 256 |
| 455 | NSLATMV | 5491 | AATAGTCTGGCGACGATGGTG | 5492 | 89 |
| 456 | RSISGDW | 5493 | CGTTCCATAAGTGGCGACTGG | 5494 | 159 |
| 457 | SLQQANT | 5495 | TCGCTTCAGCAGGCTAATACG | 5496 | 87 |
| 458 | PTTNPLL | 5497 | CCGACTACTAATCCGCTTCTG | 5498 | 56 |
| 459 | ADVLIRG | 5499 | GCGGACGTGCTCATACGCGGT | 5500 | 269 |
| 460 | HVASAGA | 5501 | CATGTTGCTTCGGCGGGGGCG | 5502 | 253 |
| 461 | LQDRTTL | 5503 | CTCCAAGACCGCACTACTCTC | 5504 | 204 |
| 462 | NAHDTET | 5505 | AATGCGCATGATACTGAGACT | 5506 | 246 |
| 463 | RVDSALL | 5507 | AGAGTAGACAGCGCTCTTTTA | 5508 | 86 |
| 464 | RNQGSES | 5509 | CGTAATCAGGGTAGTGAGAGT | 5510 | 252 |
| 465 | EIMSSNR | 5511 | GAAATCATGTCGTCCAACCGT | 5512 | 249 |
| 466 | GSRENAR | 5513 | GGGAGTAGGGAGAATGCGCGT | 5514 | 70 |
| 467 | GGDTSRS | 5515 | GGGGGTGATACGAGTCGTAGT | 5516 | 335 |
| 468 | YLALTGI | 5517 | TATCTTGCGCTTACGGGGATT | 5518 | 78 |
| 469 | LDTSARL | 5519 | CTTGATACTAGTGCTCGTCTT | 5520 | 248 |
| 470 | LLTLTQA | 5521 | CTGCTCACCCTGACTCAAGCG | 5522 | 247 |
| 471 | SEQNKVW | 5523 | TCCGAACAAAACAAAGTATGG | 5524 | 94 |
| 472 | ADAAHAL | 5525 | GCGGACGCAGCCCACGCGCTC | 5526 | 245 |
| 473 | GVAATNS | 5527 | GGGGTGGCTGCGACGAATTCT | 5528 | 84 |
| 474 | NSGSMHT | 5529 | AACTCAGGAAGCATGCACACT | 5530 | 293 |
| 475 | PDGAAPM | 5531 | CCTGATGGTGCGGCTCCTATG | 5532 | 341 |
| 476 | STLASPR | 5533 | TCAACCCTAGCCTCGCCTCGA | 5534 | 244 |
| 477 | GADDAAL | 5535 | GGAGCCGACGACGCAGCCCTC | 5536 | 315 |
| 478 | AGASAEA | 5537 | GCTGGGGCTAGTGCTGAGGCG | 5538 | 228 |
| 479 | SRLEYIG | 5539 | AGCCGCCTTGAATACATCGGG | 5540 | 349 |
| 480 | YTVGSLA | 5541 | TACACCGTTGGCTCACTCGCC | 5542 | 314 |
| 481 | LVHLGTS | 5543 | TTGGTTCATCTTGGGACTTCT | 5544 | 198 |
| 482 | GLYDAAT | 5545 | GGGCTTTATGATGCGGCGACT | 5546 | 315 |
| 483 | KNGGHDL | 5547 | AAAAACGGTGGGCACGACCTA | 5548 | 268 |
| 484 | NTENASR | 5549 | AATACTGAGAATGCGTCGCGG | 5550 | 242 |
| 485 | HGTLVSQ | 5551 | CATGGGACTTTGGTGTCTCAG | 5552 | 178 |
| 486 | HAGLGVT | 5553 | CATGCTGGTCTTGGTGTTACT | 5554 | 163 |
| 487 | PSYQGNG | 5555 | CCGAGTTATCAGGGGAATGGT | 5556 | 181 |
| 488 | MGDNYAR | 5557 | ATGGGTGATAATTATGCTCGG | 5558 | 10 |
| 489 | LEKDPMT | 5559 | TTGGAAAAAGACCCTATGACT | 5560 | 318 |
| 490 | SMNGTSL | 5561 | AGTATGAATGGGACTAGTCTT | 5562 | 81 |
| 491 | SNLGNTS | 5563 | AGTAACCTTGGAAACACCTCG | 5564 | 241 |
| 492 | GSGAGLH | 5565 | GGAAGTGGAGCTGGCCTTCAC | 5566 | 172 |
| 493 | LANTVVT | 5567 | CTTGCTAATACGGTTGTGACG | 5568 | 80 |
| 494 | AVRENGI | 5569 | GCCGTTCGGGAAAACGGCATA | 5570 | 34 |
| 495 | VTELTRF | 5571 | GTGACTGAGCTTACGCGGTTT | 5572 | 162 |
| 496 | RNLDLNH | 5573 | AGGAATCTTGATCTGAATCAT | 5574 | 261 |
| 497 | ALASTQT | 5575 | GCACTAGCATCGACCCAAACT | 5576 | 79 |
| 498 | FISGALT | 5577 | TTCATATCCGGCGCCTTAACT | 5578 | 240 |
| 499 | QSQTAVA | 5579 | CAGTCTCAGACGGCTGTTGCT | 5580 | 1 |
| 500 | VSSQLPM | 5581 | GTGTCGTCTCAGTTGCCGATG | 5582 | 239 |
| 501 | VIALTEA | 5583 | GTGATTGCGTTGACGGAGGCT | 5584 | 78 |
| 502 | TTVEVSG | 5585 | ACAACCGTAGAAGTAAGCGGC | 5586 | 236 |
| 503 | GVAGTNS | 5587 | GGAGTTGCGGGAACAAACTCC | 5588 | 84 |
| 504 | RESGEQA | 5589 | AGGGAGAGTGGGGAGCAGGCT | 5590 | 233 |
| 505 | HIVLSHA | 5591 | CATATTGTGCTGAGTCATGCT | 5592 | 78 |
| 506 | ILGVYSD | 5593 | ATACTGGGCGTTTACTCCGAC | 5594 | 287 |
| 507 | QGGTTLR | 5595 | CAAGGGGGGACTACTCTACGC | 5596 | 49 |
| 508 | YHTEKMF | 5597 | TACCACACCGAAAAAATGTTC | 5598 | 320 |
| 509 | LEVGALR | 5599 | CTGGAAGTAGGCGCACTTCGT | 5600 | 231 |
| 510 | GFGLTED | 5601 | GGGTTTGGGTTGACGGAGGAT | 5602 | 322 |
| 511 | PLKGGGE | 5603 | CCGTTGAAAGGCGGGGGTGAA | 5604 | 328 |
| 512 | GLVHMPS | 5605 | GGCTTAGTTCACATGCCCTCA | 5606 | 326 |
| 513 | VTGHPTL | 5607 | GTTACGGGTCATCCGACTCTT | 5608 | 0 |
| 514 | WNHSTTV | 5609 | TGGAACCACTCCACGACAGTC | 5610 | 2 |
| 515 | PGEHYRL | 5611 | CCTGGAGAACACTACAGATTG | 5612 | 196 |
| 516 | LSLTDLV | 5613 | CTGAGTTTGACTGATTTGGTT | 5614 | 230 |
| 517 | GTGSTNV | 5615 | GGAACTGGATCGACAAACGTT | 5616 | 229 |
| 518 | LSKEHAP | 5617 | TTGAGTAAGGAGCATGCTCCT | 5618 | 57 |
| 519 | LGDSAEA | 5619 | CTTGGGGATTCTGCTGAGGCG | 5620 | 228 |
| 520 | GPRNSID | 5621 | GGCCCACGTAACTCTATCGAC | 5622 | 343 |
| 521 | LVRGLTT | 5623 | TTGGTTCGTGGTCTTACGACT | 5624 | 222 |
| 522 | REVSPLM | 5625 | CGAGAAGTAAGCCCCCTGATG | 5626 | 215 |
| 523 | IMPSVTK | 5627 | ATAATGCCCTCTGTTACAAAA | 5628 | 136 |
| 524 | WNSEVSV | 5629 | TGGAACAGTGAAGTTTCGGTG | 5630 | 320 |
| 525 | FHGSDSK | 5631 | TTTCATGGTTCTGATTCGAAG | 5632 | 174 |
| 526 | PNERLTQ | 5633 | CCGAATGAGAGGCTTACTCAG | 5634 | 144 |
| 527 | GQEETGW | 5635 | GGGCAGGAGGAGACCGGCTGG | 5636 | 169 |
| 528 | MVTTTNT | 5637 | ATGGTGACGACCACAAACACC | 5638 | 77 |
| 529 | NALGDGY | 5639 | AACGCGCTGGGCGACGGCTAC | 5640 | 312 |
| 530 | LGDSAET | 5641 | CTTGGGGATTCTGCTGAGACG | 5642 | 219 |
| 531 | TGAHTEV | 5643 | ACCGGAGCACACACCGAAGTC | 5644 | 82 |
| 532 | LVGNPST | 5645 | CTCGTGGGCAACCCGAGTACG | 5646 | 287 |
| 533 | FPSMSGK | 5647 | TTCCCAAGCATGTCGGGGAAA | 5648 | 62 |
| 534 | KGSDTPL | 5649 | AAGGGTTCTGATACTCCTTTG | 5650 | 278 |
| 535 | ANLGESV | 5651 | GCCAACCTCGGTGAATCCGTG | 5652 | 218 |
| 536 | SVDSGLR | 5653 | AGTGTTGATAGTGGGCTGCGT | 5654 | 217 |
| 537 | RTMGDST | 5655 | CGGACAATGGGTGACAGTACG | 5656 | 312 |
| 538 | SLAISQR | 5657 | AGCCTGGCTATAAGCCAACGT | 5658 | 76 |
| 539 | SEISLSR | 5659 | TCTGAGATTAGTCTGTCTCGG | 5660 | 75 |
| 540 | LRGTENQ | 5661 | TTGCGTGGGACGGAGAATCAG | 5662 | 237 |
| 541 | SGHVTAL | 5663 | AGTGGACACGTCACAGCTTTA | 5664 | 114 |
| 542 | REISILS | 5665 | CGCGAAATATCGATACTATCT | 5666 | 215 |
| 543 | ASTDFKM | 5667 | GCTAGTACTGATTTTAAGATG | 5668 | 330 |
| 544 | GNSGDHF | 5669 | GGTAACTCTGGTGACCACTTC | 5670 | 149 |
| 545 | AADSSVR | 5671 | GCTGCTGACAGCAGCGTTAGA | 5672 | 73 |
| 546 | QADSHGR | 5673 | CAAGCCGACTCGCACGGCCGT | 5674 | 62 |
| 547 | ADYGTSS | 5675 | GCGGACTACGGTACCAGCTCT | 5676 | 108 |
| 548 | SGGVESK | 5677 | TCTGGTGGTGTTGAGTCGAAG | 5678 | 310 |
| 549 | GNLLLTA | 5679 | GGTAATTTGCTGCTTACTGCT | 5680 | 115 |
| 550 | MTDRHRV | 5681 | ATGACCGACCGTCACAGGGTC | 5682 | 187 |
| 551 | MENAPGR | 5683 | ATGGAGAATGCTCCTGGGAGG | 5684 | 62 |
| 552 | EANHTGY | 5685 | GAAGCCAACCACACCGGATAC | 5686 | 254 |
| 553 | AADRSVR | 5687 | GCAGCAGACCGCTCCGTACGT | 5688 | 72 |
| 554 | PIIEHAV | 5689 | CCCATAATAGAACACGCAGTA | 5690 | 330 |
| 555 | NVDTSVR | 5691 | AATGTTGATACGAGTGTGCGG | 5692 | 213 |
| 556 | NVTATLG | 5693 | AACGTCACAGCAACGCTGGGT | 5694 | 203 |
| 557 | MKTQIEL | 5695 | ATGAAAACGCAAATAGAACTC | 5696 | 126 |
| 558 | IGPRREV | 5697 | ATAGGACCTCGCCGTGAAGTA | 5698 | 82 |
| 559 | VLAAVDR | 5699 | GTCCTTGCTGCCGTCGACCGA | 5700 | 211 |
| 560 | SVDSGLL | 5701 | AGTGTTGATAGTGGGCTGCTT | 5702 | 210 |
| 561 | FIVGNGS | 5703 | TTCATCGTAGGCAACGGAAGT | 5704 | 22 |
| 562 | RYNVETA | 5705 | CGGTATAATGTTGAGACTGCG | 5706 | 333 |
| 563 | AIVSIAQ | 5707 | GCGATTGTGTCGATTGCTCAG | 5708 | 71 |
| 564 | PDNNPRN | 5709 | CCTGATAATAATCCGCGGAAT | 5710 | 19 |
| 565 | KTVNVSV | 5711 | AAGACTGTGAATGTTAGTGTT | 5712 | 69 |
| 566 | QFHENIR | 5713 | CAGTTTCATGAGAATATTCGT | 5714 | 153 |
| 567 | KGYDTPM | 5715 | AAAGGCTACGACACACCCATG | 5716 | 278 |
| 568 | AVITEPK | 5717 | GCGGTGATTACTGAGCCTAAG | 5718 | 207 |
| 569 | VSSTGEW | 5719 | GTTAGTTCTACGGGGGAGTGG | 5720 | 198 |
| 570 | TGSIPSP | 5721 | ACCGGTTCAATCCCTTCCCCC | 5722 | 184 |
| 571 | NQSAELV | 5723 | AATCAGTCGGCGGAGCTGGTT | 5724 | 209 |
| 572 | VIGGLGI | 5725 | GTTATTGGTGGGCTTGGGATT | 5726 | 212 |
| 573 | HFSSETS | 5727 | CACTTCTCTTCCGAAACTTCT | 5728 | 65 |
| 574 | GYRGVVD | 5729 | GGTTATAGGGGGGTTGTGGAT | 5730 | 149 |
| 575 | SHGTDTK | 5731 | AGCCACGGAACGGACACCAAA | 5732 | 174 |
| 576 | VMASPGP | 5733 | GTTATGGCTTCGCCTGGTCCT | 5734 | 176 |
| 577 | LPNGGGL | 5735 | CTGCCGAATGGGGGGGGTTTG | 5736 | 30 |
| 578 | VGSVTDS | 5737 | GTTGGTAGCGTAACCGACTCC | 5738 | 206 |
| 579 | TVMTSEP | 5739 | ACAGTTATGACCAGCGAACCT | 5740 | 159 |
| 580 | PGNGTMV | 5741 | CCTGGTAACGGCACTATGGTG | 5742 | 128 |
| 581 | SLGALVA | 5743 | TCGCTGGGTGCTCTGGTTGCT | 5744 | 68 |
| 582 | YLVTADN | 5745 | TATTTGGTTACTGCTGATAAT | 5746 | 45 |
| 583 | LTHLRVS | 5747 | CTGACTCACCTTCGTGTCAGC | 5748 | 305 |
| 584 | HTVGSYV | 5749 | CATACGGTTGGGAGTTATGTT | 5750 | 216 |
| 585 | LEDRSAS | 5751 | TTGGAGGATCGGTCGGCTAGT | 5752 | 204 |
| 586 | VTTASPV | 5753 | GTGACTACGGCTTCTCCTGTG | 5754 | 67 |
| 587 | GVLGQTD | 5755 | GGTGTGTTGGGGCAGACTGAT | 5756 | 149 |
| 588 | DIDRLHK | 5757 | GATATTGATAGGCTGCATAAG | 5758 | 12 |
| 589 | HNPGMDK | 5759 | CATAATCCGGGGATGGATAAG | 5760 | 337 |
| 590 | TVGLTIA | 5761 | ACTGTGGGTTTGACGATTGCG | 5762 | 201 |
| 591 | SPPPNAR | 5763 | AGCCCGCCGCCGAACGCGCGT | 5764 | 183 |
| 592 | FLLGHTD | 5765 | TTCCTTCTGGGGCACACGGAC | 5766 | 119 |
| 593 | VLTSPGP | 5767 | GTGCTCACAAGCCCGGGACCG | 5768 | 176 |
| 594 | TAYDTLV | 5769 | ACGGCGTATGATACGTTGGTT | 5770 | 339 |
| 595 | SVETGVL | 5771 | TCTGTGGAAACTGGCGTCTTA | 5772 | 200 |
| 596 | TVKEYEL | 5773 | ACCGTTAAAGAATACGAACTC | 5774 | 277 |
| 597 | MTVPGSP | 5775 | ATGACGGTTCCGGGTAGTCCG | 5776 | 101 |
| 598 | YYSITSS | 5777 | TACTACTCCATCACATCCAGT | 5778 | 88 |
| 599 | STIPTLK | 5779 | AGTACTATTCCTACTCTGAAG | 5780 | 332 |
| 600 | MVQSGLT | 5781 | ATGGTTCAGTCGGGGTTGACG | 5782 | 170 |
| 601 | MGVGGGS | 5783 | ATGGGGGTCGGTGGTGGATCC | 5784 | 110 |
| 602 | RVDSGQL | 5785 | AGGGTGGATTCGGGGCAGCTT | 5786 | 186 |
| 603 | REISNLR | 5787 | CGGGAAATAAGCAACCTACGT | 5788 | 188 |
| 604 | DHVLLTR | 5789 | GACCACGTGTTACTTACCCGG | 5790 | 75 |
| 605 | TRIGLSD | 5791 | ACACGAATAGGACTCAGTGAC | 5792 | 323 |
| 606 | RVHSAQL | 5793 | AGGGTGCATTCGGCGCAGCTT | 5794 | 302 |
| 607 | YEHSGLL | 5795 | TATGAGCATTCTGGTCTTTTG | 5796 | 170 |
| 608 | VFTGTDT | 5797 | GTGTTCACAGGAACCGACACA | 5798 | 129 |
| 609 | TLAINER | 5799 | ACTTTGGCGATTAATGAGCGG | 5800 | 85 |
| 610 | LGVTNVA | 5801 | CTAGGAGTGACCAACGTGGCC | 5802 | 340 |
| 611 | GVANVSQ | 5803 | GGTGTGGCGAATGTGAGTCAG | 5804 | 197 |
| 612 | ATVKDSG | 5805 | GCAACCGTAAAAGACTCGGGG | 5806 | 236 |
| 613 | GEIDIAF | 5807 | GGAGAAATCGACATAGCCTTC | 5808 | 75 |
| 614 | LSLTDGV | 5809 | TTGTCCTTAACCGACGGAGTG | 5810 | 259 |
| 615 | VLLMDRV | 5811 | GTACTTCTTATGGACCGAGTT | 5812 | 296 |
| 616 | SSADYQV | 5813 | AGTTCTGCGGATTATCAGGTT | 5814 | 330 |
| 617 | APRDPGV | 5815 | GCGCCGCGTGATCCTGGTGTT | 5816 | 327 |
| 618 | AQAQTGW | 5817 | GCTCAAGCACAGACCGGCTGG | 5818 | 169 |
| 619 | SNLHTST | 5819 | AGTAATCTTCATACTTCGACT | 5820 | 303 |
| 620 | RVDSGLL | 5821 | AGGGTTGATAGTGGGCTGCTT | 5822 | 86 |
| 621 | SGGRITD | 5823 | AGCGGAGGGCGCATCACCGAC | 5824 | 179 |
| 622 | LGIGQGP | 5825 | TTGGGTATTGGTCAGGGTCCT | 5826 | 184 |
| 623 | MGGVTSV | 5827 | ATGGGGGGGGTTACTTCGGTG | 5828 | 195 |
| 624 | SIYDNVK | 5829 | TCGATATACGACAACGTCAAA | 5830 | 339 |
| 625 | VTSDAGW | 5831 | GTCACCTCTGACGCAGGGTGG | 5832 | 309 |
| 626 | AHTEMSH | 5833 | GCCCACACCGAAATGTCTCAC | 5834 | 261 |
| 627 | LLTQDAR | 5835 | TTGCTTACTCAGGATGCTCGG | 5836 | 193 |
| 628 | YVGSPLV | 5837 | TATGTTGGTTCTCCGTTGGTG | 5838 | 111 |
| 629 | ENAGTDV | 5839 | GAAAACGCCGGAACTGACGTC | 5840 | 29 |
| 630 | SIYDNDT | 5841 | TCCATCTACGACAACGACACC | 5842 | 289 |
| 631 | AATSGGP | 5843 | GCAGCCACCAGTGGCGGGCCG | 5844 | 62 |
| 632 | STIPTLM | 5845 | TCGACGATACCAACCTTGATG | 5846 | 308 |
| 633 | SGMQAEA | 5847 | TCGGGTATGCAGGCGGAGGCT | 5848 | 192 |
| 634 | GNGDMFA | 5849 | GGGAATGGGGATATGTTTGCT | 5850 | 264 |
| 635 | LNGGIGV | 5851 | CTTAATGGGGGTATTGGGGTT | 5852 | 164 |
| 636 | TAVERAW | 5853 | ACGGCTGTTGAGCGGGCGTGG | 5854 | 205 |
| 637 | NNGIVIA | 5855 | AATAATGGGATTGTGATTGCG | 5856 | 305 |
| 638 | GPDTGAM | 5857 | GGCCCCGACACAGGCGCGATG | 5858 | 315 |
| 639 | PSRGIPL | 5859 | CCGAGTCGTGGTATTCCTCTT | 5860 | 341 |
| 640 | VGGAGEI | 5861 | GTTGGTGGGGCGGGTGAGATT | 5862 | 101 |
| 641 | VLQLAAL | 5863 | GTTCTTCAACTCGCTGCCCTC | 5864 | 66 |
| 642 | LSDGGPL | 5865 | CTCTCGGACGGAGGCCCCCTC | 5866 | 286 |
| 643 | VSGGVLD | 5867 | GTATCCGGCGGAGTACTAGAC | 5868 | 161 |
| 644 | MSITEPR | 5869 | ATGTCTATTACTGAGCCGCGG | 5870 | 207 |
| 645 | SGSNTGP | 5871 | AGCGGCTCCAACACTGGCCCG | 5872 | 184 |
| 646 | SLRDTHY | 5873 | AGTCTTCGGGATACTCATTAT | 5874 | 318 |
| 647 | MGDAGLR | 5875 | ATGGGGGATGCGGGGCTGCGG | 5876 | 217 |
| 648 | IVMSSHI | 5877 | ATCGTCATGAGCTCCCACATC | 5878 | 189 |
| 649 | ASPLPQT | 5879 | GCTAGTCCCTTGCCCCAAACC | 5880 | 122 |
| 650 | SEISILR | 5881 | AGTGAGATTAGTATTCTGCGG | 5882 | 188 |
| 651 | SEGLSRD | 5883 | TCGGAGGGTCTTTCGCGTGAT | 5884 | 306 |
| 652 | LGSLVVH | 5885 | CTGGGAAGCTTAGTCGTTCAC | 5886 | 114 |
| 653 | ETRLDSK | 5887 | GAAACCCGACTCGACTCGAAA | 5888 | 54 |
| 654 | ANQLAPV | 5889 | GCCAACCAATTGGCCCCCGTG | 5890 | 272 |
| 655 | LFGPSAY | 5891 | TTATTCGGACCTTCCGCCTAC | 5892 | 317 |
| 656 | SMTSESS | 5893 | TCAATGACTTCGGAATCGTCT | 5894 | 65 |
| 657 | MTDSGTV | 5895 | ATGACTGATAGTGGGACTGTG | 5896 | 187 |
| 658 | FQVEQIM | 5897 | TTTCAGGTTGAGCAGATTATG | 5898 | 329 |
| 659 | RVDSEQL | 5899 | AGGGTGGATTCGGAGCAGCTT | 5900 | 146 |
| 660 | SNTGVTV | 5901 | TCGAATACTGGTGTTACGGTG | 5902 | 180 |
| 661 | ITQAVYI | 5903 | ATCACACAAGCGGTATACATC | 5904 | 300 |
| 662 | GALSSTK | 5905 | GGTGCTCTTTCTTCGACTAAG | 5906 | 63 |
| 663 | IMVDAHS | 5907 | ATTATGGTTGATGCTCATTCG | 5908 | 175 |
| 664 | GSGVQPV | 5909 | GGGTCCGGCGTACAACCGGTA | 5910 | 264 |
| 665 | GSGPGVA | 5911 | GGTTCTGGGCCGGGGGTGGCT | 5912 | 163 |
| 666 | KHSSEMT | 5913 | AAACACAGCTCAGAAATGACC | 5914 | 346 |
| 667 | TRTEDYT | 5915 | ACGCGTACGGAGGATTATACT | 5916 | 349 |
| 668 | ILNPTAV | 5917 | ATTCTTAATCCGACGGCGGTG | 5918 | 5 |
| 669 | IGSSLSP | 5919 | ATTGGGTCGTCGCTTAGTCCT | 5920 | 184 |
| 670 | SGFVVPV | 5921 | TCTGGGTTTGTTGTGCCGGTG | 5922 | 114 |
| 671 | RTTPDVT | 5923 | CGCACGACCCCCGACGTAACA | 5924 | 107 |
| 672 | APTATLR | 5925 | GCGCCTACTGCTACTCTTCGG | 5926 | 183 |
| 673 | YDRIMSS | 5927 | TACGACCGCATAATGTCATCT | 5928 | 168 |
| 674 | YGSNDLS | 5929 | TATGGGAGTAATGATCTGAGT | 5930 | 110 |
| 675 | GDRGVVA | 5931 | GGTGATAGGGGGGTTGTGGCT | 5932 | 53 |
| 676 | QAALSDR | 5933 | CAAGCGGCACTATCAGACCGG | 5934 | 182 |
| 677 | AADSSGR | 5935 | GCGGCGGATAGTTCTGGGCGG | 5936 | 62 |
| 678 | PTLGTLR | 5937 | CCTACTCTGGGGACGCTTCGG | 5938 | 128 |
| 679 | PNLGNPS | 5939 | CCCAACCTCGGAAACCCATCT | 5940 | 241 |
| 680 | PTQGTNR | 5941 | CCAACACAAGGTACAAACAGG | 5942 | 128 |
| 681 | SRGVISS | 5943 | AGCCGAGGCGTAATCTCGTCA | 5944 | 310 |
| 682 | ASVSSLR | 5945 | GCTAGTGTGTCTTCGCTGCGT | 5946 | 61 |
| 683 | LRVTEDL | 5947 | TTGCGTGTGACGGAGGATCTG | 5948 | 237 |
| 684 | MTGLDDV | 5949 | ATGACGGGCCTAGACGACGTA | 5950 | 142 |
| 685 | TSLGPMV | 5951 | ACTTCGTTAGGCCCGATGGTC | 5952 | 323 |
| 686 | MAGGVQV | 5953 | ATGGCGGGTGGGGTGCAGGTT | 5954 | 177 |
| 687 | NGASLAS | 5955 | AACGGAGCTTCCCTCGCAAGC | 5956 | 59 |
| 688 | TNGVLYT | 5957 | ACAAACGGCGTCCTTTACACG | 5958 | 93 |
| 689 | MNGGHVQ | 5959 | ATGAACGGAGGGCACGTGCAA | 5960 | 268 |
| 690 | VMASTGP | 5961 | GTAATGGCGTCAACAGGACCG | 5962 | 176 |
| 691 | VLASLGD | 5963 | GTACTCGCGTCGTTGGGCGAC | 5964 | 212 |
| 692 | ILVDALA | 5965 | ATTCTGGTTGATGCTCTTGCG | 5966 | 175 |
| 693 | SADSSVR | 5967 | TCGGCGGATAGTTCTGTGCGG | 5968 | 58 |
| 694 | NRELALG | 5969 | AACCGCGAACTCGCACTCGGG | 5970 | 263 |
| 695 | SHASDSK | 5971 | TCGCACGCATCAGACTCTAAA | 5972 | 174 |
| 696 | AGHSNAV | 5973 | GCTGGGCATTCTAATGCGGTT | 5974 | 158 |
| 697 | MVTPTNS | 5975 | ATGGTGACGCCGACCAACAGT | 5976 | 77 |
| 698 | TIDRFGS | 5977 | ACAATAGACCGATTCGGAAGT | 5978 | 204 |
| 699 | RGAEVLL | 5979 | CGGGGTGCGGAGGTGCTGCTG | 5980 | 313 |
| 700 | TFAISDR | 5981 | ACTTTTGCGATTTCTGATCGG | 5982 | 262 |
| 701 | SQGSDSK | 5983 | AGTCAAGGCTCCGACTCAAAA | 5984 | 54 |
| 702 | ASGVRPV | 5985 | GCGTCAGGTGTTAGACCGGTA | 5986 | 272 |
| 703 | SDATGVL | 5987 | TCCGACGCTACCGGTGTGCTA | 5988 | 44 |
| 704 | LTLSNGV | 5989 | CTTACGCTGAGTAATGGGGTG | 5990 | 171 |
| 705 | DSDSGRR | 5991 | GATTCTGATAGTGGGCGGCGG | 5992 | 217 |
| 706 | LYTSDRV | 5993 | CTATACACATCTGACCGAGTG | 5994 | 248 |
| 707 | QDAHVAI | 5995 | CAGGATGCGCATGTGGCTATT | 5996 | 0 |
| 708 | IVDSGLL | 5997 | ATTGTTGATAGTGGGCTGCTT | 5998 | 170 |
| 709 | LYGGSSA | 5999 | CTCTACGGAGGGTCCTCGGCT | 6000 | 317 |
| 710 | NFGRDTL | 6001 | AATTTTGGTCGTGATACTCTG | 6002 | 174 |
| 711 | TPVYTVK | 6003 | ACCCCCGTCTACACCGTAAAA | 6004 | 145 |
| 712 | AAVVPRY | 6005 | GCAGCAGTAGTACCACGATAC | 6006 | 122 |
| 713 | SNVALTG | 6007 | AGCAACGTTGCACTGACCGGC | 6008 | 64 |
| 714 | ASMGTVA | 6009 | GCGTCCATGGGAACCGTAGCC | 6010 | 53 |
| 715 | TIGVVAN | 6011 | ACGATTGGGGTTGTGGCGAAT | 6012 | 137 |
| 716 | TSVLPQT | 6013 | ACGTCTGTGCTTCCTCAGACT | 6014 | 292 |
| 717 | LHAGESR | 6015 | CTTCATGCTGGTGAGTCTAGG | 6016 | 134 |
| 718 | STTSSPR | 6017 | TCTACGACGAGTTCGCCGCGT | 6018 | 52 |
| 719 | PGHGPVR | 6019 | CCCGGGCACGGACCTGTACGC | 6020 | 128 |
| 720 | FTSGTGN | 6021 | TTCACAAGCGGGACCGGAAAC | 6022 | 112 |
| 721 | QILGASS | 6023 | CAAATCTTAGGGGCCTCGAGT | 6024 | 51 |
| 722 | EVRDTKT | 6025 | GAAGTTCGGGACACAAAAACG | 6026 | 246 |
| 723 | VLPSPGP | 6027 | GTTCTGCCTTCGCCTGGTCCT | 6028 | 176 |
| 724 | INNFAPP | 6029 | ATAAACAACTTCGCACCGCCC | 6030 | 139 |
| 725 | ELRPQSS | 6031 | GAACTCCGGCCCCAATCATCT | 6032 | 65 |
| 726 | LTDKMTS | 6033 | TTGACTGATAAGATGACGTCG | 6034 | 334 |
| 727 | IYPQSST | 6035 | ATATACCCACAAAGCTCCACC | 6036 | 317 |
| 728 | VVSGLLH | 6037 | GTTGTCTCCGGGTTGCTACAC | 6038 | 238 |
| 729 | ATVAGQY | 6039 | GCTACCGTGGCAGGCCAATAC | 6040 | 101 |
| 730 | NLGGVQL | 6041 | AACTTAGGAGGCGTCCAATTG | 6042 | 177 |
| 731 | SEPSGTL | 6043 | AGCGAACCCTCCGGAACTTTA | 6044 | 25 |
| 732 | MNGGHVI | 6045 | ATGAATGGGGGTCATGTTATT | 6046 | 268 |
| 733 | VVNVGQT | 6047 | GTAGTGAACGTCGGACAAACT | 6048 | 198 |
| 734 | GLTEYTA | 6049 | GGTCTAACCGAATACACAGCT | 6050 | 331 |
| 735 | ILASPGP | 6051 | ATACTTGCGTCACCCGGACCG | 6052 | 176 |
| 736 | VGSVMAS | 6053 | GTGGGGTCGGTTATGGCTAGT | 6054 | 168 |
| 737 | SPQGVLA | 6055 | TCGCCGCAGGGGGTTCTTGCT | 6056 | 274 |
| 738 | VGPSVLQ | 6057 | GTAGGTCCATCCGTACTACAA | 6058 | 161 |
| 739 | GVRDTNI | 6059 | GGAGTTCGAGACACAAACATA | 6060 | 132 |
| 740 | ALQSAQV | 6061 | GCACTACAATCTGCACAAGTT | 6062 | 50 |
| 741 | GGVSATA | 6063 | GGAGGAGTCAGCGCAACGGCT | 6064 | 165 |
| 742 | NPSPTET | 6065 | AACCCTAGCCCGACCGAAACC | 6066 | 311 |
| 743 | AIVSIAR | 6067 | GCGATTGTGTCGATTGCTCGG | 6068 | 48 |
| 744 | AVPREGM | 6069 | GCCGTCCCGCGCGAAGGAATG | 6070 | 34 |
| 745 | PGAHYQA | 6071 | CCGGGTGCGCATTATCAGGCT | 6072 | 196 |
| 746 | SPPSSQR | 6073 | TCACCCCCTTCATCCCAACGC | 6074 | 58 |
| 747 | RSNTGEW | 6075 | CGGTCAAACACCGGCGAATGG | 6076 | 163 |
| 748 | HLYTGTG | 6077 | CACTTATACACTGGCACCGGA | 6078 | 44 |
| 749 | NGPMKAD | 6079 | AACGGTCCAATGAAAGCAGAC | 6080 | 59 |
| 750 | AADTSVR | 6081 | GCGGCGGATACTTCTGTGCGG | 6082 | 47 |
| 751 | GLEKMTS | 6083 | GGTCTGGAGAAGATGACTTCT | 6084 | 326 |
| 752 | IIISSAN | 6085 | ATAATCATATCCTCGGCCAAC | 6086 | 45 |
| 753 | GLVKMPT | 6087 | GGTCTGGTGAAGATGCCTACT | 6088 | 326 |
| 754 | SLPPYGR | 6089 | AGCCTGCCCCCCTACGGCCGT | 6090 | 279 |
| 755 | TSLGLMQ | 6091 | ACTAGCCTTGGCTTAATGCAA | 6092 | 323 |
| 756 | LSRGAEN | 6093 | CTTTCGAGGGGTGCGGAGAAT | 6094 | 324 |
| 757 | AVKEYEL | 6095 | GCCGTTAAAGAATACGAACTC | 6096 | 277 |
| 758 | TTPSPRT | 6097 | ACGACCCCTAGCCCACGAACA | 6098 | 292 |
| 759 | HGTLVSK | 6099 | CACGGCACCCTTGTTTCCAAA | 6100 | 178 |
| 760 | VTELTQV | 6101 | GTCACCGAACTCACACAAGTC | 6102 | 162 |
| 761 | NGNMATF | 6103 | AATGGGAATATGGCGACTTTT | 6104 | 336 |
| 762 | EGGDSGG | 6105 | GAAGGCGGAGACAGCGGTGGA | 6106 | 49 |
| 763 | MGDIVTL | 6107 | ATGGGGGATATTGTTACGCTT | 6108 | 95 |
| 764 | LIVTENQ | 6109 | TTGATTGTGACGGAGAATCAG | 6110 | 237 |
| 765 | SVATGVL | 6111 | AGCGTGGCTACAGGCGTGCTC | 6112 | 44 |
| 766 | VSPSVLQ | 6113 | GTTAGTCCTTCGGTGCTTCAG | 6114 | 161 |
| 767 | VTGLTVQ | 6115 | GTTACCGGGCTGACAGTACAA | 6116 | 160 |
| 768 | ASQDRGS | 6117 | GCATCTCAAGACCGGGGCTCT | 6118 | 327 |
| 769 | SSVSSPR | 6119 | TCCAGCGTCTCCTCTCCTCGC | 6120 | 43 |
| 770 | PILGAST | 6121 | CCGATTCTTGGTGCTAGTACG | 6122 | 328 |
| 771 | SQLSVML | 6123 | AGCCAACTTTCAGTAATGCTT | 6124 | 42 |
| 772 | TDALTSK | 6125 | ACAGACGCACTCACCAGTAAA | 6126 | 157 |
| 773 | YLEGTLL | 6127 | TACCTGGAAGGGACATTGCTC | 6128 | 113 |
| 774 | YQRTESL | 6129 | TATCAGAGGACGGAGTCTCTG | 6130 | 320 |
| 775 | QGGGSLN | 6131 | CAGGGGGGGGGTAGTCTGAAT | 6132 | 49 |
| 776 | PGSEIRG | 6133 | CCTGGCTCCGAAATAAGAGGC | 6134 | 33 |
| 777 | PSRGITL | 6135 | CCGAGTCGTGGTATTACTCTT | 6136 | 341 |
| 778 | GVAGTDS | 6137 | GGGGTGGCTGGGACGGATTCT | 6138 | 156 |
| 779 | MNGGHVM | 6139 | ATGAACGGTGGACACGTGATG | 6140 | 268 |
| 780 | TVPNTDL | 6141 | ACTGTGCCTAATACTGATTTG | 6142 | 60 |
| 781 | GHQALNA | 6143 | GGCCACCAAGCATTAAACGCC | 6144 | 235 |
| 782 | DPKTGWR | 6145 | GATCCGAAGACTGGGTGGCGT | 6146 | 316 |
| 783 | VTQAVYV | 6147 | GTTACGCAGGCTGTTTATGTT | 6148 | 300 |
| 784 | FETGGVS | 6149 | TTCGAAACCGGAGGCGTTTCC | 6150 | 240 |
| 785 | IADMGGN | 6151 | ATTGCTGATATGGGTGGTAAT | 6152 | 62 |
| 786 | PGYSSQT | 6153 | CCGGGGTATAGTTCTCAGACG | 6154 | 158 |
| 787 | LLLGVQS | 6155 | CTCCTATTAGGAGTACAATCG | 6156 | 155 |
| 788 | AVDSSVR | 6157 | GCTGTTGACTCCAGCGTTAGA | 6158 | 40 |
| 789 | YESTRGQ | 6159 | TATGAGTCGACGAGGGGTCAG | 6160 | 151 |
| 790 | LNSPLHV | 6161 | CTGAATAGTCCGCTGCATGTT | 6162 | 112 |
| 791 | ADTAHPV | 6163 | GCCGACACCGCCCACCCCGTT | 6164 | 245 |
| 792 | LPKGGGF | 6165 | CTGCCGAAGGGGGGGGGGTTT | 6166 | 30 |
| 793 | EGVSALL | 6167 | GAGGGTGTTTCTGCGTTGTTG | 6168 | 154 |
| 794 | PNERLTL | 6169 | CCAAACGAACGTTTGACCTTA | 6170 | 144 |
| 795 | SGGLMTG | 6171 | AGTGGTGGTCTTATGACTGGT | 6172 | 179 |
| 796 | VIETRLS | 6173 | GTCATCGAAACTCGCCTTTCC | 6174 | 152 |
| 797 | LANMLQV | 6175 | TTGGCAAACATGCTTCAAGTG | 6176 | 167 |
| 798 | SPTSSPH | 6177 | TCACCTACATCCTCACCACAC | 6178 | 52 |
| 799 | GVGGTYS | 6179 | GGAGTTGGGGGCACATACAGT | 6180 | 234 |
| 800 | AAESSVR | 6181 | GCGGCGGAGAGTTCTGTGCGG | 6182 | 38 |
| 801 | MNDAGRD | 6183 | ATGAATGATGCTGGGCGTGAT | 6184 | 286 |
| 802 | GISGEVS | 6185 | GGTATTTCGGGGGAGGTGAGT | 6186 | 149 |
| 803 | PQLIVPK | 6187 | CCTCAGCTTATTGTTCCTAAG | 6188 | 244 |
| 804 | LRVTENQ | 6189 | TTGCGTGTGACGGAGAATCAG | 6190 | 237 |
| 805 | TSPGLMV | 6191 | ACATCACCCGGCCTGATGGTT | 6192 | 323 |
| 806 | TTAAIDR | 6193 | ACCACTGCAGCCATCGACCGA | 6194 | 148 |
| 807 | HGNGYLS | 6195 | CACGGAAACGGGTACCTTTCA | 6196 | 110 |
| 808 | VVSDYTV | 6197 | GTTGTTAGTGATTATACTGTG | 6198 | 330 |
| 809 | RLAITER | 6199 | AGATTGGCGATTACTGAGCGG | 6200 | 147 |
| 810 | PGVDTGV | 6201 | CCTGGTGTTGATACTGGTGTT | 6202 | 99 |
| 811 | DTSASST | 6203 | GATACGTCGGCGTCGTCGACT | 6204 | 37 |
| 812 | ANEHNIA | 6205 | GCTAATGAGCATAATATTGCG | 6206 | 338 |
| 813 | IAHGYST | 6207 | ATCGCCCACGGATACAGCACA | 6208 | 222 |
| 814 | SLAISER | 6209 | AGCTTAGCCATCAGCGAAAGG | 6210 | 36 |
| 815 | RDLTTDL | 6211 | CGTGATCTGACGACTGATCTG | 6212 | 159 |
| 816 | EASSRLL | 6213 | GAAGCTTCGTCGCGACTTCTC | 6214 | 35 |
| 817 | AVKEYQS | 6215 | GCTGTTAAAGAATACCAATCT | 6216 | 277 |
| 818 | GIAVGEV | 6217 | GGTATTGCTGTGGGGGAGGTT | 6218 | 44 |
| 819 | RSITIGP | 6219 | CGTTCGATTACTATTGGGCCG | 6220 | 133 |
| 820 | LGDGTTR | 6221 | CTGGGGGATGGTACGACTCGG | 6222 | 255 |
| 821 | ALMSSGV | 6223 | GCGTTGATGTCCTCGGGGGTT | 6224 | 34 |
| 822 | TYSDGTT | 6225 | ACTTATAGTGATGGGACGACT | 6226 | 90 |
| 823 | ASGEVQS | 6227 | GCGTCGGGGGAGGTTCAGTCT | 6228 | 33 |
| 824 | FAGVQQA | 6229 | TTCGCAGGAGTCCAACAAGCT | 6230 | 287 |
| 825 | ESSRLQI | 6231 | GAGAGTTCGCGTCTTCAGATT | 6232 | 239 |
| 826 | DSGKDRT | 6233 | GATTCTGGTAAGGATCGTACG | 6234 | 37 |
| 827 | MLALAVT | 6235 | ATGTTGGCGCTGGCTGTGACG | 6236 | 32 |
| 828 | GERMGMT | 6237 | GGTGAGCGGATGGGTATGACT | 6238 | 270 |
| 829 | MADGASM | 6239 | ATGGCGGATGGTGCGTCTATG | 6240 | 255 |
| 830 | RHLTSDV | 6241 | CGACACCTCACATCCGACGTC | 6242 | 159 |
| 831 | EVLSLAP | 6243 | GAGGTGCTGTCTCTTGCTCCG | 6244 | 109 |
| 832 | DIAVSMR | 6245 | GACATCGCGGTATCGATGAGA | 6246 | 143 |
| 833 | RSAGTST | 6247 | AGGTCTGCAGGAACCTCCACA | 6248 | 29 |
| 834 | TSYDTVV | 6249 | ACATCATACGACACCGTCGTG | 6250 | 339 |
| 835 | VGASTAW | 6251 | GTGGGCGCCAGCACCGCGTGG | 6252 | 88 |
| 836 | RVELTGT | 6253 | CGCGTAGAATTGACCGGCACG | 6254 | 295 |
| 837 | VQGPLTG | 6255 | GTGCAGGGTCCGCTGACTGGT | 6256 | 14 |
| 838 | DRVISSL | 6257 | GATCGGGTTATTAGTTCTTTG | 6258 | 37 |
| 839 | PLILSPS | 6259 | CCCTTGATCTTATCTCCAAGT | 6260 | 117 |
| 840 | VRQLDSR | 6261 | GTGAGGCAGCTGGATTCGCGG | 6262 | 27 |
| 841 | LLAGADR | 6263 | TTGCTTGCTGGTGCTGATCGT | 6264 | 140 |
| 842 | YSTERSV | 6265 | TATTCGACTGAGAGGTCTGTT | 6266 | 320 |
| 843 | GPMASVV | 6267 | GGGCCGATGGCGTCTGTGGTT | 6268 | 216 |
| 844 | MLGGGAS | 6269 | ATGCTCGGCGGAGGTGCCTCC | 6270 | 298 |
| 845 | LRGQPGV | 6271 | CTGCGCGGCCAACCCGGCGTG | 6272 | 164 |
| 846 | GNGTRVL | 6273 | GGAAACGGCACCAGGGTCCTA | 6274 | 172 |
| 847 | GLVQIVA | 6275 | GGGCTTGTTCAGATTGTTGCG | 6276 | 326 |
| 848 | SFRDTVP | 6277 | AGTTTTAGGGATACGGTGCCT | 6278 | 318 |
| 849 | SLNSVKV | 6279 | TCCCTAAACTCGGTCAAAGTG | 6280 | 28 |
| 850 | HLSRDHS | 6281 | CACCTGTCACGTGACCACTCA | 6282 | 127 |
| 851 | SGDRDQN | 6283 | TCTGGTGATCGGGATCAGAAT | 6284 | 251 |
| 852 | SGPMKAV | 6285 | AGTGGGCCGATGAAGGCGGTT | 6286 | 304 |
| 853 | AGGGTPR | 6287 | GCGGGGGGTGGGACTCCGAGG | 6288 | 49 |
| 854 | NLRGEHT | 6289 | AATTTGCGTGGGGAGCATACG | 6290 | 131 |
| 855 | GGTGEGP | 6291 | GGTGGGACGGGTGAGGGTCCG | 6292 | 149 |
| 856 | LRVPENQ | 6293 | TTGCGTGTGCCGGAGAATCAG | 6294 | 237 |
| 857 | AAGLILN | 6295 | GCCGCAGGCCTCATCCTTAAC | 6296 | 179 |
| 858 | TGERDQN | 6297 | ACTGGTGAACGGGATCAGAAT | 6298 | 251 |
| 859 | TIAAHVP | 6299 | ACCATAGCAGCCCACGTACCC | 6300 | 91 |
| 860 | SFAITER | 6301 | AGTTTTGCGATTACTGAGCGG | 6302 | 322 |
| 861 | FTIKDNR | 6303 | TTCACCATAAAAGACAACAGA | 6304 | 237 |
| 862 | ESRENVR | 6305 | GAATCCCGTGAAAACGTCAGA | 6306 | 316 |
| 863 | LPRLGGL | 6307 | CTTCCGCGTTTGGGGGGGCTT | 6308 | 30 |
| 864 | GPDTGAK | 6309 | GGTCCAGACACAGGAGCCAAA | 6310 | 10 |
| 865 | TGGLLYS | 6311 | ACTGGTGGGCTTCTTTATAGT | 6312 | 179 |
| 866 | RSGSGVA | 6313 | CGGTCGGGCTCCGGAGTCGCC | 6314 | 163 |
| 867 | LTGSIGL | 6315 | TTAACTGGGTCAATTGGACTC | 6316 | 164 |
| 868 | TLPHAGL | 6317 | ACCCTCCCCCACGCAGGGTTA | 6318 | 34 |
| 869 | VDHGMGL | 6319 | GTTGATCATGGTATGGGTTTG | 6320 | 212 |
| 870 | TVELNHV | 6321 | ACGGTTGAGCTGAATCATGTT | 6322 | 162 |
| 871 | VLSSDLR | 6323 | GTGCTTTCGAGTGATCTTCGT | 6324 | 118 |
| 872 | TLTYTET | 6325 | ACATTGACATACACTGAAACC | 6326 | 79 |
| 873 | FIDSQLG | 6327 | TTTATTGATAGTCAGCTGGGT | 6328 | 152 |
| 874 | FSTNSNH | 6329 | TTCTCGACCAACAGCAACCAC | 6330 | 138 |
| 875 | MGASDTL | 6331 | ATGGGGGCTAGTGATACGCTT | 6332 | 26 |
| 876 | TTGKVSG | 6333 | ACCACGGGTAAAGTGTCGGGG | 6334 | 236 |
| 877 | MDELRGR | 6335 | ATGGACGAATTACGCGGCAGA | 6336 | 157 |
| 878 | SVDNGLL | 6337 | TCCGTCGACAACGGCTTACTG | 6338 | 210 |
| 879 | TGMQVSI | 6339 | ACCGGTATGCAAGTGTCGATC | 6340 | 190 |
| 880 | SVVSGLL | 6341 | TCTGTGGTGTCAGGTCTTTTG | 6342 | 25 |
| 881 | EQYLGSP | 6343 | GAGCAGTATCTGGGTTCTCCG | 6344 | 74 |
| 882 | LSHTEGD | 6345 | TTATCACACACCGAAGGGGAC | 6346 | 259 |
| 883 | DFSVAHT | 6347 | GACTTCTCTGTAGCGCACACT | 6348 | 37 |
| 884 | MATPTNT | 6349 | ATGGCAACGCCAACTAACACC | 6350 | 77 |
| 885 | NTEDRRV | 6351 | AACACAGAAGACCGGCGAGTT | 6352 | 242 |
| 886 | AGVLKAL | 6353 | GCAGGAGTATTAAAAGCCCTC | 6354 | 304 |
| 887 | NAHALMV | 6355 | AACGCCCACGCACTCATGGTC | 6356 | 89 |
| 888 | VHVDNSN | 6357 | GTGCATGTTGATAATAGTAAT | 6358 | 45 |
| 889 | LSIRQGP | 6359 | TTGAGTATTCGTCAGGGTCCT | 6360 | 259 |
| 890 | GVNHAVA | 6361 | GGAGTCAACCACGCCGTCGCC | 6362 | 4 |
| 891 | AYVTQGG | 6363 | GCCTACGTAACACAAGGCGGC | 6364 | 345 |
| 892 | WDDQTSG | 6365 | TGGGATGATCAGACTTCGGGG | 6366 | 204 |
| 893 | LDLTSDV | 6367 | CTTGATCTGACGTCTGATGTG | 6368 | 159 |
| 894 | IPSDFPN | 6369 | ATACCATCCGACTTCCCGAAC | 6370 | 271 |
| 895 | SLVRGLL | 6371 | AGTCTTGTTCGGGGTTTGCTG | 6372 | 25 |
| 896 | IVYAVGE | 6373 | ATAGTCTACGCTGTTGGAGAA | 6374 | 133 |
| 897 | LAGLGGM | 6375 | CTAGCTGGCCTCGGTGGAATG | 6376 | 164 |
| 898 | SDEAYRA | 6377 | AGCGATGAGGCTTATCGTGCG | 6378 | 245 |
| 899 | VGQVPGR | 6379 | GTGGGGCAAGTCCCGGGTAGG | 6380 | 168 |
| 900 | SVDSALL | 6381 | TCCGTGGACTCTGCTTTGCTG | 6382 | 24 |
| 901 | LSLRDGV | 6383 | CTATCCCTTAGGGACGGAGTC | 6384 | 259 |
| 902 | STIPTPM | 6385 | TCCACAATCCCAACCCCCATG | 6386 | 308 |
| 903 | IPRIHSL | 6387 | ATTCCTCGGATTCATTCTCTT | 6388 | 245 |
| 904 | LSGIMVS | 6389 | TTGTCGGGGATTATGGTTTCG | 6390 | 305 |
| 905 | GFVQSRM | 6391 | GGGTTTGTTCAGAGTCGGATG | 6392 | 326 |
| 906 | SSQGTTK | 6393 | TCTTCGCAGGGTACGACTAAG | 6394 | 23 |
| 907 | NHVGDRL | 6395 | AATCATGTTGGTGATCGTTTG | 6396 | 226 |
| 908 | VESTAFT | 6397 | GTTGAGAGTACGGCTTTTACG | 6398 | 214 |
| 909 | LVAGQAM | 6399 | CTGGTGGCGGGGCAGGCTATG | 6400 | 21 |
| 910 | GLVRIQD | 6401 | GGACTGGTTCGGATCCAAGAC | 6402 | 326 |
| 911 | TNTDSSL | 6403 | ACGAATACGGATTCTAGTCTG | 6404 | 20 |
| 912 | TGLQVST | 6405 | ACTGGGCTGCAGGTTAGTACT | 6406 | 227 |
| 913 | AHGDKDL | 6407 | GCACACGGCGACAAAGACCTT | 6408 | 291 |
| 914 | SSANLSN | 6409 | TCGTCCGCCAACCTTTCGAAC | 6410 | 19 |
| 915 | EIAFTVP | 6411 | GAAATAGCATTCACCGTACCT | 6412 | 120 |
| 916 | SGEPLGL | 6413 | TCTGGGGAGCCGCTTGGGCTT | 6414 | 279 |
| 917 | QNVGVTK | 6415 | CAAAACGTAGGAGTTACGAAA | 6416 | 64 |
| 918 | LSNLSNG | 6417 | CTTAGTAATCTGTCGAATGGT | 6418 | 247 |
| 919 | LSTGEEM | 6419 | CTTTCGACGGGTGAGGAGATG | 6420 | 232 |
| 920 | EGGGAQR | 6421 | GAGGGGGGTGGGGCTCAGAGG | 6422 | 49 |
| 921 | DVRGSVN | 6423 | GACGTCCGGGGGTCTGTCAAC | 6424 | 149 |
| 922 | LNGDTGY | 6425 | CTTAATGGTGATACGGGGTAT | 6426 | 164 |
| 923 | MGDNYDR | 6427 | ATGGGCGACAACTACGACCGC | 6428 | 228 |
| 924 | QLRPLQT | 6429 | CAACTGCGTCCTTTGCAAACG | 6430 | 318 |
| 925 | AGVMNDL | 6431 | GCCGGTGTTATGAACGACCTT | 6432 | 304 |
| 926 | LLENARV | 6433 | CTGCTGGAGAATGCGAGGGTG | 6434 | 243 |
| 927 | LVVDASR | 6435 | TTGGTAGTAGACGCAAGTCGC | 6436 | 18 |
| 928 | LATHDAR | 6437 | CTCGCAACGCACGACGCACGA | 6438 | 193 |
| 929 | VRQLDSN | 6439 | GTAAGACAACTTGACTCTAAC | 6440 | 66 |
| 930 | QARDTKT | 6441 | CAAGCTCGAGACACCAAAACA | 6442 | 246 |
| 931 | TGDREQN | 6443 | ACTGGTGATCGGGAACAGAAT | 6444 | 251 |
| 932 | SGAAAAT | 6445 | AGCGGGGCCGCAGCCGCCACC | 6446 | 17 |
| 933 | GRKGEGP | 6447 | GGTAGGAAGGGTGAGGGTCCG | 6448 | 149 |
| 934 | QNVGVTQ | 6449 | CAGAATGTGGGGGTGACTCAG | 6450 | 64 |
| 935 | DSAPAAR | 6451 | GATTCGGCTCCGGCGGCTCGG | 6452 | 16 |
| 936 | ANQNVII | 6453 | GCAAACCAAAACGTAATAATA | 6454 | 265 |
| 937 | GAHIVSA | 6455 | GGGGCGCACATAGTCTCCGCA | 6456 | 106 |
| 938 | NSDLASP | 6457 | AATAGTGATTTGGCGTCTCCT | 6458 | 242 |
| 939 | AASMVVG | 6459 | GCTGCGAGTATGGTTGTTGGG | 6460 | 269 |
| 940 | VVSEIPL | 6461 | GTCGTTAGCGAAATCCCCCTC | 6462 | 271 |
| 941 | QAESAAR | 6463 | CAAGCGGAATCAGCGGCTAGA | 6464 | 16 |
| 942 | LSKEHAH | 6465 | TTATCGAAAGAACACGCCCAC | 6466 | 57 |
| 943 | TNLADTA | 6467 | ACTAATCTGGCTGATACTGCG | 6468 | 273 |
| 944 | ADREVRY | 6469 | GCGGATCGGGAGGTGCGTTAT | 6470 | 15 |
| 945 | NISVTPV | 6471 | AATATTAGTGTTACGCCGGTT | 6472 | 276 |
| 946 | PSRGNEG | 6473 | CCCAGTCGCGGGAACGAAGGC | 6474 | 294 |
| 947 | MMLNQGS | 6475 | ATGATGCTTAACCAAGGCAGC | 6476 | 259 |
| 948 | VHSQDVS | 6477 | GTGCATTCGCAGGATGTGTCT | 6478 | 346 |
| 949 | ANAEVQR | 6479 | GCGAATGCGGAGGTTCAGCGT | 6480 | 15 |
| 950 | LGPGITL | 6481 | TTAGGCCCCGGTATCACCCTC | 6482 | 95 |
| 951 | NVAELVA | 6483 | AACGTCGCAGAATTGGTGGCA | 6484 | 288 |
| 952 | ILSGLTS | 6485 | ATTCTGAGTGGGTTGACTTCT | 6486 | 14 |
| 953 | VNVSPTT | 6487 | GTGAATGTTAGTCCTACTACT | 6488 | 13 |
| 954 | GERDARI | 6489 | GGTGAGAGGGATGCTAGGATT | 6490 | 315 |
| 955 | IGMSAST | 6491 | ATAGGTATGAGCGCGTCCACC | 6492 | 13 |
| 956 | LSRGEEK | 6493 | CTTTCGAGGGGTGAGGAGAAG | 6494 | 294 |
| 957 | KNKGVDP | 6495 | AAAAACAAAGGCGTCGACCCA | 6496 | 337 |
| 958 | HQDRTTL | 6497 | CATCAGGATAGGACGACGCTT | 6498 | 144 |
| 959 | RISTEGT | 6499 | CGCATCAGCACAGAAGGCACT | 6500 | 295 |
| 960 | ALSGLAK | 6501 | GCACTGTCCGGACTCGCAAAA | 6502 | 12 |
| 961 | IGASVKL | 6503 | ATCGGTGCATCGGTAAAACTG | 6504 | 11 |
| 962 | ISLNAAE | 6505 | ATTTCGCTGAATGCGGCGGAG | 6506 | 9 |
| 963 | SHGSDTK | 6507 | TCCCACGGAAGTGACACCAAA | 6508 | 174 |
| 964 | HGRDALV | 6509 | CATGGGCGGGATGCTCTTGTG | 6510 | 154 |
| 965 | GVAGTYL | 6511 | GGGGTGGCTGGGACGTATCTG | 6512 | 234 |
| 966 | STEGAAL | 6513 | AGTACGGAGGGGGCGGCTCTG | 6514 | 8 |
| 967 | SVMGVVR | 6515 | TCCGTCATGGGAGTAGTTCGT | 6516 | 7 |
| 968 | SVVVTAR | 6517 | TCGGTCGTCGTAACAGCTCGG | 6518 | 6 |
| 969 | PLVGAPV | 6519 | CCGCTGGTTGGGGCTCCGGTT | 6520 | 328 |
| 970 | MGGATNP | 6521 | ATGGGGGGGGCTACTAATCCG | 6522 | 195 |
| 971 | NGPMEAV | 6523 | AACGGACCAATGGAAGCAGTC | 6524 | 333 |
| 972 | QVTDTKT | 6525 | CAGGTGACTGATACTAAGACT | 6526 | 246 |
| 973 | NSKDVQR | 6527 | AACTCCAAAGACGTACAAAGA | 6528 | 15 |
| 974 | RTTEPRF | 6529 | CGTACTACGGAGCCTCGTTTT | 6530 | 248 |
| 975 | VVGLTAA | 6531 | GTTGTCGGCTTAACCGCAGCG | 6532 | 5 |
| 976 | PGEHYQV | 6533 | CCTGGCGAACACTACCAAGTG | 6534 | 196 |
| 977 | RAVENMG | 6535 | CGCGCAGTAGAAAACATGGGC | 6536 | 349 |
| 978 | RVMGEEV | 6537 | CGTGTGATGGGGGAGGAGGTT | 6538 | 312 |
| 979 | KYSGAES | 6539 | AAATACTCTGGCGCGGAATCT | 6540 | 348 |
| 980 | MLVTETV | 6541 | ATGTTGGTCACTGAAACGGTA | 6542 | 259 |
| 981 | VFVEKSA | 6543 | GTTTTTGTTGAGAAGAGTGCG | 6544 | 344 |
| 982 | SVNQAVT | 6545 | TCTGTGAATCAGGCGGTTACG | 6546 | 4 |
| 983 | GHSATAA | 6547 | GGACACTCCGCTACCGCCGCA | 6548 | 235 |
| 984 | HDTSASV | 6549 | CATGATACTAGTGCTAGTGTT | 6550 | 223 |
| 985 | SVDSGLI | 6551 | TCCGTAGACTCCGGACTTATC | 6552 | 220 |
| 986 | VGKVMDV | 6553 | GTCGGAAAAGTCATGGACGTC | 6554 | 206 |
| 987 | AIVSIAK | 6555 | GCCATCGTTTCAATAGCAAAA | 6556 | 3 |
| 988 | PAEHYQA | 6557 | CCGGCTGAGCATTATCAGGCT | 6558 | 196 |
| 989 | VSLTDGL | 6559 | GTGAGTTTGACTGATGGGCTT | 6560 | 259 |
| 990 | SVTDVNH | 6561 | TCTGTTACTGATGTTAATCAT | 6562 | 261 |
| 991 | GLNEHEA | 6563 | GGTCTGAATGAGCATGAGGCG | 6564 | 331 |
| 992 | ANVGRDD | 6565 | GCAAACGTTGGCCGCGACGAC | 6566 | 218 |
| 993 | AGYSSLT | 6567 | GCTGGATACTCGTCACTCACA | 6568 | 158 |
| 994 | QLQPQQT | 6569 | CAACTCCAACCCCAACAAACC | 6570 | 50 |
| 995 | MRVTENQ | 6571 | ATGAGGGTCACTGAAAACCAA | 6572 | 237 |
| 996 | ITEQTTI | 6573 | ATTACTGAGCAGACTACTATT | 6574 | 2 |
| 997 | VVDSDNL | 6575 | GTTGTTGATTCGGATAATCTG | 6576 | 141 |
| 998 | FSTDTSS | 6577 | TTTTCGACGGATACGTCGTCT | 6578 | 138 |

Macaque all CNS_sequence Rank

| | | SEQ | | |
|---|---|---|---|---|
| | | ID | | |
| Rank | Peptide | NO: | Sequence | SEQ ID NO: |
| 1 | PSQGTLR | 6579 | CCTTCTCAGGGGACGCTTCGG | 6580 |
| 2 | PTQGTVR | 6581 | CCCACACAAGGCACAGTCCGT | 6582 |
| 3 | TDALTTK | 6583 | ACTGATGCGCTTACGACTAAG | 6584 |
| 4 | TDAGDGK | 6585 | ACAGACGCGGGGGACGGCAAA | 6586 |
| 5 | MTGISIV | 6587 | ATGACAGGCATCTCTATCGTA | 6588 |
| 6 | NGYTEGR | 6589 | AATGGGTATACGGAGGGGCGT | 6590 |
| 7 | PTQGTVR | 6591 | CCTACTCAGGGGACGGTTCGG | 6592 |
| 8 | SLVTSST | 6593 | TCGCTTGTTACTTCTAGTACG | 6594 |
| 9 | PTQGTFR | 6595 | CCGACACAAGGAACATTCAGG | 6596 |
| 10 | PTQGTIR | 6597 | CCTACTCAGGGGACGATTCGG | 6598 |
| 11 | AIVSIAQ | 6599 | GCGATTGTGTCGATTGCTCAG | 6600 |
| 12 | LTSGLAA | 6601 | TTGACGTCTGGTTTGGCGGCG | 6602 |
| 13 | PTQGTFR | 6603 | CCTACTCAGGGGACGTTTCGG | 6604 |
| 14 | STIPTMK | 6605 | AGTACTATTCCTACTATGAAG | 6606 |
| 15 | GTVGSMV | 6607 | GGTACGGTGGGTTCTATGGTT | 6608 |
| 16 | ELMASTI | 6609 | GAGCTTATGGCTTCTACTATT | 6610 |
| 17 | SPVGIIA | 6611 | TCTCCTGTGGGTATTATTGCG | 6612 |
| 18 | TSREEQW | 6613 | ACTTCTCGTGAGGAGCAGTGG | 6614 |
| 19 | RASADVV | 6615 | AGGGCGAGTGCGGATGTTGTG | 6616 |
| 20 | RYNDEST | 6617 | AGATACAACGACGAATCCACT | 6618 |
| 21 | HTIAASM | 6619 | CACACCATAGCCGCAAGTATG | 6620 |
| 22 | HTQGTLR | 6621 | CATACTCAGGGGACGCTTCGG | 6622 |
| 23 | DGRAELR | 6623 | GATGGGCGGGCGGAGTTGCGT | 6624 |
| 24 | AADSSAR | 6625 | GCCGCTGACTCATCGGCCCGT | 6626 |
| 25 | NLGAALS | 6627 | AACCTTGGGGCTGCCCTATCG | 6628 |
| 26 | ALNEHVA | 6629 | GCTCTGAATGAGCATGTGGCG | 6630 |
| 27 | IMVDAHS | 6631 | ATTATGGTTGATGCTCATTCG | 6632 |
| 28 | PAQGTLR | 6633 | CCGGCGCAAGGAACACTACGA | 6634 |
| 29 | SIGDLGK | 6635 | AGTATCGGTGACCTAGGTAAA | 6636 |
| 30 | PSQGTLR | 6637 | CCGTCCCAAGGAACACTCAGG | 6638 |
| 31 | PTHGTLR | 6639 | CCGACCCACGGTACACTGCGA | 6640 |
| 32 | NRELALG | 6641 | AACCGCGAACTCGCACTCGGG | 6642 |
| 33 | AGGGDPR | 6643 | GCTGGTGGAGGTGACCCCCGA | 6644 |
| 34 | PNERPTV | 6645 | CCCAACGAACGTCCAACGGTC | 6646 |
| 35 | AGVSASL | 6647 | GCGGGTGTTTCTGCGTCGTTG | 6648 |
| 36 | VIAGVGI | 6649 | GTAATCGCGGGAGTAGGCATC | 6650 |
| 37 | AIQTNDA | 6651 | GCAATCCAAACCAACGACGCG | 6652 |
| 38 | RTMGDST | 6653 | CGGACAATGGGTGACAGTACG | 6654 |
| 39 | KNQDMQV | 6655 | AAGAATCAGGATATGCAGGTG | 6656 |
| 40 | NGNMATF | 6657 | AATGGGAATATGGCGACTTTT | 6658 |
| 41 | MVTHTNK | 6659 | ATGGTAACACACACAAACAAA | 6660 |
| 42 | SLLLTTP | 6661 | TCATTACTATTGACGACACCC | 6662 |
| 43 | SSVQGIL | 6663 | TCCTCAGTCCAAGGAATACTA | 6664 |
| 44 | RIVDSVP | 6665 | AGGATTGTGGATAGTGTTCCG | 6666 |
| 45 | PTEGTLR | 6667 | CCGACAGAAGGCACACTGCGA | 6668 |
| 46 | YLVTTEN | 6669 | TATTTGGTTACTACTGAGAAT | 6670 |
| 47 | PTQGTLR | 6671 | CCTACTCAGGGGACGCTTCGG | 6672 |
| 48 | LLAGADR | 6673 | TTGCTTGCTGGTGCTGATCGT | 6674 |
| 49 | HSKGFDY | 6675 | CACAGTAAAGGTTTCGACTAC | 6676 |
| 50 | FQVEQVK | 6677 | TTTCAGGTTGAGCAGGTTAAG | 6678 |
| 51 | TGGRDQY | 6679 | ACTGGTGGTCGGGATCAGTAT | 6680 |
| 52 | PTPGTLR | 6681 | CCTACTCCGGGGACGCTTCGG | 6682 |
| 53 | LGDSAEA | 6683 | CTTGGGGATTCTGCTGAGGCG | 6684 |
| 54 | RVDSEQH | 6685 | AGGGTGGATTCGGAGCAGCAT | 6686 |
| 55 | SVDSGML | 6687 | AGTGTTGATAGTGGGATGCTT | 6688 |
| 56 | NTENASR | 6689 | AATACTGAGAATGCGTCGCGG | 6690 |
| 57 | PTLGTLR | 6691 | CCTACTCTGGGGACGCTTCGG | 6692 |
| 58 | RVALDLP | 6693 | AGGGTGGCGCTGGATTTGCCG | 6694 |
| 59 | AGDRDQY | 6695 | GCTGGTGATCGGGATCAGTAT | 6696 |
| 60 | VIALTEA | 6697 | GTGATTGCGTTGACGGAGGCT | 6698 |
| 61 | STIHTLK | 6699 | AGTACTATTCATACTCTGAAG | 6700 |
| 62 | REALALT | 6701 | AGGGAGGCGCTGGCTCTGACG | 6702 |
| 63 | TTSGNLM | 6703 | ACGACGTCGGGGAATCTTATG | 6704 |
| 64 | LTLSNGV | 6705 | CTTACGCTGAGTAATGGGGTG | 6706 |
| 65 | GVVNDER | 6707 | GGCGTTGTCAACGACGAACGG | 6708 |
| 66 | DKVVDEV | 6709 | GATAAGGTGGTTGATGAGGTG | 6710 |
| 67 | MAASVTL | 6711 | ATGGCGGCTAGTGTTACGCTT | 6712 |
| 68 | LTGERIL | 6713 | CTAACCGGTGAACGCATACTT | 6714 |
| 69 | NTVVNDP | 6715 | AACACAGTCGTGAACGACCCT | 6716 |
| 70 | VNAALGI | 6717 | GTTAATGCTGCGCTTGGGATT | 6718 |
| 71 | SRELTGS | 6719 | TCGAGGGAGTTGACTGGGTCG | 6720 |
| 72 | TVDSPMR | 6721 | ACCGTCGACAGCCCTATGCGA | 6722 |
| 73 | ATDSSVR | 6723 | GCCACCGACAGCAGTGTCCGT | 6724 |
| 74 | LESLSHH | 6725 | CTGGAGTCGCTTTCTCATCAT | 6726 |
| 75 | ALGTQGS | 6727 | GCTCTGGGTACGCAGGGTTCT | 6728 |
| 76 | RTTPDVP | 6729 | CGTACGACTCCCGACGTACCT | 6730 |
| 77 | VLNDNLA | 6731 | GTGTTAAACGACAACTTAGCT | 6732 |
| 78 | CNAAGCP | 6733 | TGTAATGCTGCGGGGTGTCCG | 6734 |
| 79 | LHAGESR | 6735 | CTTCATGCTGGTGAGTCTAGG | 6736 |
| 80 | HAGLGVT | 6737 | CATGCTGGTCTTGGTGTTACT | 6738 |
| 81 | KIGENAS | 6739 | AAGATTGGTGAGAATGCTTCT | 6740 |
| 82 | NAHDTET | 6741 | AATGCGCATGATACTGAGACT | 6742 |
| 83 | TGLQDSN | 6743 | ACAGGATTGCAAGACTCGAAC | 6744 |
| 84 | ASNPGRW | 6745 | GCGAGTAACCCTGGAAGGTGG | 6746 |
| 85 | VVGTQDR | 6747 | GTTGTGGGGACTCAGGATAGG | 6748 |
| 86 | VHASSPT | 6749 | GTGCATGCTTCTAGTCCGACT | 6750 |
| 87 | NGLQVSI | 6751 | AATGGGCTGCAGGTTAGTATT | 6752 |
| 88 | TVPNTDL | 6753 | ACTGTGCCTAATACTGATTTG | 6754 |
| 89 | FLLGHTD | 6755 | TTCCTTCTGGGGCACACGGAC | 6756 |
| 90 | REISILS | 6757 | CGCGAAATATCGATACTATCT | 6758 |
| 91 | DSLLPQT | 6759 | GATTCTCTGCTTCCTCAGACT | 6760 |
| 92 | RGGVTTE | 6761 | CGTGGAGGCGTAACCACCGAA | 6762 |
| 93 | HVASAGA | 6763 | CATGTTGCTTCGGCGGGGGCG | 6764 |
| 94 | FGGVINA | 6765 | TTCGGGGGAGTAATAAACGCT | 6766 |
| 95 | NGMGDVT | 6767 | AACGGCATGGGGGACGTTACT | 6768 |
| 96 | TTVEVSG | 6769 | ACAACCGTAGAAGTAAGCGGC | 6770 |
| 97 | VGGNVVH | 6771 | GTTGGTGGTAATGTTGTTCAT | 6772 |
| 98 | PMRPGVA | 6773 | CCGATGCGGCCGGGTGTGGCT | 6774 |
| 99 | RESGEQA | 6775 | AGGGAGAGTGGGGAGCAGGCT | 6776 |
| 100 | LSLTEGV | 6777 | CTGAGTTTGACTGAGGGGGTT | 6778 |
| 101 | PKPSHGE | 6779 | CCTAAACCATCTCACGGAGAA | 6780 |
| 102 | YQRTESL | 6781 | TATCAGAGGACGGAGTCTCTG | 6782 |
| 103 | PGEHYRL | 6783 | CCTGGAGAACACTACAGATTG | 6784 |
| 104 | TSVESNL | 6785 | ACGTCGGTGGAGTCGAATCTT | 6786 |
| 105 | GSGAGLH | 6787 | GGAAGTGGAGCTGGCCTTCAC | 6788 |
| 106 | MAGGTNP | 6789 | ATGGCAGGTGGCACAAACCCT | 6790 |
| 107 | RESLEAL | 6791 | AGGGAGAGTCTTGAGGCGTTG | 6792 |
| 108 | KGYDTPM | 6793 | AAAGGCTACGACACACCCATG | 6794 |
| 109 | DQLNDGR | 6795 | GATCAGCTGAATGATGGGCGG | 6796 |
| 110 | TIGVVAN | 6797 | ACGATTGGGGTTGTGGCGAAT | 6798 |
| 111 | LSTGSQL | 6799 | CTGTCTACGGGGTCGCAGCTG | 6800 |
| 112 | VPGSTTT | 6801 | GTTCCAGGCTCAACGACTACC | 6802 |
| 113 | MGDAGLR | 6803 | ATGGGGGATGCGGGGCTGCGG | 6804 |
| 114 | SPTSSPH | 6805 | TCACCTACATCCTCACCACAC | 6806 |
| 115 | ESLAGVR | 6807 | GAATCGTTGGCAGGGGTGCGT | 6808 |
| 116 | KLAEGVR | 6809 | AAACTAGCCGAAGGAGTGCGG | 6810 |
| 117 | GNSGDHF | 6811 | GGTAACTCTGGTGACCACTTC | 6812 |
| 118 | AVITEPK | 6813 | GCGGTGATTACTGAGCCTAAG | 6814 |
| 119 | APTATLR | 6815 | GCGCCTACTGCTACTCTTCGG | 6816 |
| 120 | STFSTVM | 6817 | AGCACATTCTCCACTGTTATG | 6818 |
| 121 | VLASLGD | 6819 | GTACTCGCGTCGTTGGGCGAC | 6820 |
| 122 | AAGVIPN | 6821 | GCCGCCGGAGTGATACCTAAC | 6822 |
| 123 | PGEPLRL | 6823 | CCGGGAGAACCCTTGCGACTC | 6824 |
| 124 | VTSDAGW | 6825 | GTCACCTCTGACGCAGGGTGG | 6826 |
| 125 | STLISET | 6827 | TCCACGTTGATATCAGAAACC | 6828 |
| 126 | VGGAGEI | 6829 | GTTGGTGGGGCGGGTGAGATT | 6830 |
| 127 | KTAQVQP | 6831 | AAGACGGCGCAGGTGCAGCCG | 6832 |
| 128 | SMNGTSL | 6833 | AGTATGAATGGGACTAGTCTT | 6834 |
| 129 | MVGLMGA | 6835 | ATGGTGGGTCTGATGGGGGCT | 6836 |
| 130 | LNVVDLQ | 6837 | TTGAACGTTGTGGACTTGCAA | 6838 |
| 131 | SVVSGLL | 6839 | TCTGTGGTGTCAGGTCTTTTG | 6840 |
| 132 | MAGGVQV | 6841 | ATGGCGGGTGGGGTGCAGGTT | 6842 |
| 133 | SVTERSG | 6843 | TCTGTGACGGAGAGGAGTGGT | 6844 |
| 134 | PNQGTLR | 6845 | CCAAACCAAGGTACTCTACGA | 6846 |
| 135 | GLNEHEA | 6847 | GGTCTGAATGAGCATGAGGCG | 6848 |
| 136 | RVENGGT | 6849 | CGAGTGGAAAACGGCGGGACC | 6850 |
| 137 | VSLTDGL | 6851 | GTGAGTTTGACTGATGGGCTT | 6852 |
| 138 | SRLENIS | 6853 | TCGCGTCTTGAAAACATCTCC | 6854 |
| 139 | SVVPNVQ | 6855 | TCGGTGGTGCCGAATGTGCAG | 6856 |
| 140 | TSMGIMV | 6857 | ACGAGTATGGGTATTATGGTG | 6858 |
| 141 | TGLGDRA | 6859 | ACCGGCTTGGGAGACAGGGCT | 6860 |
| 142 | VGSVTDS | 6861 | GTTGGTAGCGTAACCGACTCC | 6862 |
| 143 | RTDGADH | 6863 | CGCACAGACGGAGCAGACCAC | 6864 |
| 144 | MSISEPR | 6865 | ATGTCTATTAGTGAGCCGCGG | 6866 |
| 145 | MGGVTNP | 6867 | ATGGGAGGTGTCACCAACCCC | 6868 |
| 146 | PSRGNEG | 6869 | CCCAGTCGCGGGAACGAAGGC | 6870 |
| 147 | SAGGSLQ | 6871 | AGTGCTGGTGGGAGTCTTCAG | 6872 |
| 148 | GPRNSID | 6873 | GGCCCACGTAACTCTATCGAC | 6874 |
| 149 | VLSGEEL | 6875 | GTTCTTAGTGGGGAGGAGTTG | 6876 |
| 150 | TRTEDYT | 6877 | ACGCGTACGGAGGATTATACT | 6878 |
| 151 | VTGHPTL | 6879 | GTTACGGGTCATCCGACTCTT | 6880 |
| 152 | LETVGSP | 6881 | CTGGAGACGGTTGGTTCTCCG | 6882 |
| 153 | VGRDFPA | 6883 | GTGGGTCGGGATTTTCCGGCT | 6884 |
| 154 | STQGGLA | 6885 | AGTACCCAAGGCGGATTAGCG | 6886 |
| 155 | TAVERAW | 6887 | ACGGCTGTTGAGCGGGCGTGG | 6888 |
| 156 | PLVGAPV | 6889 | CCGCTGGTTGGGGCTCCGGTT | 6890 |
| 157 | ITQAAYV | 6891 | ATCACACAAGCGGCGTACGTG | 6892 |
| 158 | LGGDVVA | 6893 | TTGGGTGGTGATGTGGTGGCG | 6894 |
| 159 | NGSSIGV | 6895 | AACGGCTCATCTATCGGCGTG | 6896 |
| 160 | TGSIPSP | 6897 | ACCGGTTCAATCCCTTCCCCC | 6898 |
| 161 | GLEKMTS | 6899 | GGTCTGGAGAAGATGACTTCT | 6900 |
| 162 | QADDHGR | 6901 | CAGGCGGATGATCATGGTAGG | 6902 |
| 163 | TMLAGSI | 6903 | ACCATGCTAGCAGGCAGCATC | 6904 |
| 164 | ASIPTLN | 6905 | GCATCCATACCAACGCTAAAC | 6906 |
| 165 | LHNLTQP | 6907 | CTTCATAATCTTACGCAGCCT | 6908 |
| 166 | LTAISDH | 6909 | CTTACGGCGATTAGTGATCAT | 6910 |
| 167 | VAALGMT | 6911 | GTTGCTGCTTTGGGTATGACT | 6912 |
| 168 | ALGDALR | 6913 | GCACTAGGCGACGCATTACGC | 6914 |
| 169 | GSGNGGS | 6915 | GGTAGTGGGAATGGTGGGAGT | 6916 |
| 170 | SADSSVR | 6917 | TCGGCGGATAGTTCTGTGCGG | 6918 |
| 171 | SVATGVL | 6919 | AGCGTGGCTACAGGCGTGCTC | 6920 |
| 172 | LVTGMSS | 6921 | CTTGTTACTGGGATGAGTTCT | 6922 |
| 173 | MVTSGLT | 6923 | ATGGTTACGTCGGGGTTGACG | 6924 |
| 174 | PREHNQA | 6925 | CCGCGTGAGCATAATCAGGCT | 6926 |
| 175 | TSPGLMV | 6927 | ACATCACCCGGCCTGATGGTT | 6928 |
| 176 | PQHIDPE | 6929 | CCTCAGCATATTGATCCTGAG | 6930 |
| 177 | GGVSATA | 6931 | GGAGGAGTCAGCGCAACGGCT | 6932 |
| 178 | SLVQGTV | 6933 | AGTCTTGTGCAGGGGACTGTT | 6934 |
| 179 | SGEPLGL | 6935 | TCTGGGGAGCCGCTTGGGCTT | 6936 |
| 180 | SQLSVML | 6937 | AGCCAACTTTCAGTAATGCTT | 6938 |
| 181 | TLTDVVH | 6939 | ACTCTTACTGATGTGGTGCAT | 6940 |
| 182 | QDGPAVK | 6941 | CAGGATGGGCCTGCGGTGAAG | 6942 |
| 183 | RGQSDPL | 6943 | CGGGGGCAGTCTGATCCGTTG | 6944 |
| 184 | IMVGTTT | 6945 | ATAATGGTAGGTACGACTACG | 6946 |
| 185 | LGSDESR | 6947 | CTGGGGTCGGATGAGAGTCGG | 6948 |
| 186 | NLGGVQL | 6949 | AACTTAGGAGGCGTCCAATTG | 6950 |
| 187 | SEQNKVW | 6951 | TCCGAACAAAACAAAGTATGG | 6952 |
| 188 | ANSHTNS | 6953 | GCAAACAGTCACACCAACTCT | 6954 |
| 189 | ATVKDSG | 6955 | GCAACCGTAAAAGACTCGGGG | 6956 |
| 190 | NGLSAST | 6957 | AATGGGCTGTCTGCTTCTACT | 6958 |
| 191 | VMASTGP | 6959 | GTAATGGCGTCAACAGGACCG | 6960 |
| 192 | VTTHSPV | 6961 | GTTACCACCCACAGTCCAGTT | 6962 |
| 193 | LSLNDVV | 6963 | CTGAGTTTGAATGATGTGGTT | 6964 |
| 194 | AHTEMSH | 6965 | GCCCACACCGAAATGTCTCAC | 6966 |
| 195 | DVAVSMI | 6967 | GATGTTGCTGTTTCTATGATT | 6968 |
| 196 | FPAGVGQ | 6969 | TTTCCGGCTGGTGTTGGGCAG | 6970 |
| 197 | VTTLSPV | 6971 | GTCACGACTTTGAGTCCAGTT | 6972 |
| 198 | LIGAALD | 6973 | CTAATCGGCGCAGCACTCGAC | 6974 |
| 199 | VLSSDLR | 6975 | GTGCTTTCGAGTGATCTTCGT | 6976 |
| 200 | QHGAEAR | 6977 | CAGCATGGGGCGGAGGCGAGG | 6978 |
| 201 | SHGSDPK | 6979 | TCTCATGGTTCTGATCCGAAG | 6980 |
| 202 | MNGGNVL | 6981 | ATGAACGGCGGCAACGTGCTC | 6982 |
| 203 | RVDSGLL | 6983 | AGGGTTGATAGTGGGCTGCTT | 6984 |
| 204 | IPSTGAQ | 6985 | ATTCCGAGTACGGGGGCGCAG | 6986 |
| 205 | VIAGLGF | 6987 | GTAATCGCAGGCTTAGGTTTC | 6988 |
| 206 | FGVSALS | 6989 | TTTGGTGTTAGTGCTCTTTCT | 6990 |
| 207 | SPAGLLA | 6991 | TCGCCGGCGGGGTTGCTTGCG | 6992 |
| 208 | STIPTPM | 6993 | TCCACAATCCCAACCCCCATG | 6994 |
| 209 | PKPSHGE | 6995 | CCGAAGCCTAGTCATGGTGAG | 6996 |
| 210 | DALSSLR | 6997 | GACGCTTTATCCAGCTTGCGA | 6998 |
| 211 | STDMRSP | 6999 | AGTACGGATATGAGGTCGCCG | 7000 |
| 212 | AVFSSQK | 7001 | GCTGTATTCTCCAGTCAAAAA | 7002 |
| 213 | RSEVNGV | 7003 | CGGAGTGAGGTGAATGGGGTT | 7004 |
| 214 | ATVAGQY | 7005 | GCTACCGTGGCAGGCCAATAC | 7006 |
| 215 | SVVVTAR | 7007 | TCGGTCGTCGTAACAGCTCGG | 7008 |
| 216 | GIDTSQP | 7009 | GGCATAGACACATCCCAACCC | 7010 |
| 217 | VVQVPGR | 7011 | GTAGTGCAAGTACCAGGACGC | 7012 |
| 218 | ITGVYDK | 7013 | ATAACTGGCGTTTACGACAAA | 7014 |
| 219 | VVDSYNL | 7015 | GTGGTAGACTCTTACAACTTA | 7016 |
| 220 | SLGEGRH | 7017 | AGTTTAGGCGAAGGGCGTCAC | 7018 |
| 221 | SIGLPAQ | 7019 | AGTATTGGGCTTCCTGCGCAG | 7020 |
| 222 | ASTVSTV | 7021 | GCCTCCACAGTAAGTACGGTC | 7022 |
| 223 | MEALAVT | 7023 | ATGGAAGCTTTGGCGGTAACA | 7024 |
| 224 | REISNLR | 7025 | CGGGAAATAAGCAACCTACGT | 7026 |
| 225 | VLDTVGN | 7027 | GTTCTGGATACGGTTGGTAAT | 7028 |
| 226 | AGLGSTS | 7029 | GCTGGTCTGGGGTCGACTAGT | 7030 |
| 227 | VEVPSTN | 7031 | GTTGAAGTCCCTTCTACGAAC | 7032 |
| 228 | VDHGGVV | 7033 | GTGGATCATGGTGGTGTGGTT | 7034 |
| 229 | GAHIVSA | 7035 | GGGGCGCACATAGTCTCCGCA | 7036 |
| 230 | TSREELR | 7037 | ACAAGTAGGGAAGAATTGCGA | 7038 |
| 231 | NGSDTTM | 7039 | AATGGTTCTGATACTACTATG | 7040 |
| 232 | KNPGVDT | 7041 | AAAAACCCTGGAGTTGACACG | 7042 |
| 233 | THDKLSV | 7043 | ACTCATGATAAGCTTAGTGTT | 7044 |
| 234 | NADYGGD | 7045 | AATGCTGATTATGGGGGTGAT | 7046 |
| 235 | ILATETS | 7047 | ATCCTAGCCACAGAAACCAGC | 7048 |
| 236 | TTMADPA | 7049 | ACAACAATGGCGGACCCCGCC | 7050 |
| 237 | VTEHTQF | 7051 | GTGACTGAGCATACGCAGTTT | 7052 |
| 238 | LTGISNV | 7053 | TTAACCGGCATCTCAAACGTA | 7054 |
| 239 | PVLAAAN | 7055 | CCTGTTCTTGCGGCAGCGAAC | 7056 |
| 240 | HATVVNS | 7057 | CATGCGACTGTTGTTAATTCG | 7058 |
| 241 | AMDNGAF | 7059 | GCTATGGATAATGGTGCTTTT | 7060 |
| 242 | SVNSIPV | 7061 | TCGGTCAACAGTATACCAGTC | 7062 |
| 243 | NLGVVPL | 7063 | AATTTGGGTGTGGTTCCGCTG | 7064 |
| 244 | QNSNGLL | 7065 | CAGAATAGTAATGGGCTTTTG | 7066 |
| 245 | ESSRLQI | 7067 | GAGAGTTCGCGTCTTCAGATT | 7068 |
| 246 | ERQLDSH | 7069 | GAGAGGCAGCTGGATTCGCAT | 7070 |
| 247 | ATTVSPV | 7071 | GCAACCACTGTGAGCCCCGTA | 7072 |
| 248 | TPPPNGR | 7073 | ACGCCTCCTCCTAATGGTAGG | 7074 |
| 249 | QDGPAVK | 7075 | CAAGACGGCCCGGCAGTTAAA | 7076 |
| 250 | VGVNGSH | 7077 | GTGGGTGTGAATGGTTCTCAT | 7078 |
| 251 | TVPNTVL | 7079 | ACAGTACCCAACACAGTCCTT | 7080 |
| 252 | HGVSIEL | 7081 | CATGGTGTTTCGATTGAGCTG | 7082 |
| 253 | SSQGTTK | 7083 | TCTTCGCAGGGTACGACTAAG | 7084 |
| 254 | TVGHDNK | 7085 | ACCGTAGGACACGACAACAAA | 7086 |
| 255 | QGGHSGG | 7087 | CAGGGTGGTCATAGTGGGGGT | 7088 |
| 256 | LTDGTVV | 7089 | CTTACTGATGGGACTGTTGTT | 7090 |
| 257 | VGASTAW | 7091 | GTGGGCGCCAGCACCGCGTGG | 7092 |
| 258 | VSRGEEM | 7093 | GTAAGCCGCGGCGAAGAAATG | 7094 |
| 259 | SIYDNDT | 7095 | TCCATCTACGACAACGACACC | 7096 |
| 260 | RETVDST | 7097 | CGTGAGACTGTGGATAGTACT | 7098 |
| 261 | STEGAAL | 7099 | AGTACGGAGGGGGCGGCTCTG | 7100 |
| 262 | ASREVIY | 7101 | GCATCGAGAGAAGTCATCTAC | 7102 |
| 263 | TEALAVK | 7103 | ACAGAAGCACTTGCGGTAAAA | 7104 |
| 264 | PTPGTLR | 7105 | CCGACACCAGGAACTTTAAGA | 7106 |
| 265 | GSGGVSV | 7107 | GGTTCGGGTGGTGTTAGTGTG | 7108 |
| 266 | PTQGVSM | 7109 | CCAACCCAAGGAGTTTCGATG | 7110 |
| 267 | VRQLDSN | 7111 | GTAAGACAACTTGACTCTAAC | 7112 |
| 268 | MGVLTTV | 7113 | ATGGGGGTGTTGACTACGGTG | 7114 |
| 269 | SLSDGSL | 7115 | TCTCTGTCTGATGGTTCTCTT | 7116 |
| 270 | HSEGVGR | 7117 | CACTCGGAAGGAGTCGGACGC | 7118 |
| 271 | WNLDMNN | 7119 | TGGAACCTAGACATGAACAAC | 7120 |
| 272 | LGHKAGD | 7121 | TTGGGGCATAAGGCTGGTGAT | 7122 |
| 273 | NLGVVNL | 7123 | AACTTAGGCGTCGTCAACCTT | 7124 |
| 274 | SGGRITD | 7125 | AGCGGAGGGCGCATCACCGAC | 7126 |
| 275 | TVITGAP | 7127 | ACTGTGATCACTGGCGCCCCC | 7128 |
| 276 | SQLAELV | 7129 | AGTCAGTTGGCGGAGCTGGTT | 7130 |
| 277 | SVTDVRH | 7131 | TCTGTTACTGATGTTAGGCAT | 7132 |
| 278 | DVAGSMR | 7133 | GATGTTGCTGGTTCTATGCGT | 7134 |
| 279 | VVQAPGR | 7135 | GTTGTTCAGGCTCCTGGGCGT | 7136 |
| 280 | NLGAALS | 7137 | AATCTGGGTGCGGCGCTTTCT | 7138 |
| 281 | KYSGAES | 7139 | AAATACTCTGGCGCGGAATCT | 7140 |
| 282 | VSATLGQ | 7141 | GTATCAGCCACACTAGGCCAA | 7142 |
| 283 | AGVSELL | 7143 | GCGGGTGTTTCTGAGTTGTTG | 7144 |
| 284 | PLLGNTI | 7145 | CCGCTTTTGGGGAATACGATT | 7146 |
| 285 | TAGLSHP | 7147 | ACCGCAGGATTGTCACACCCT | 7148 |
| 286 | RSNSAEW | 7149 | AGATCGAACTCCGCGGAATGG | 7150 |
| 287 | DTGVGTR | 7151 | GATACTGGGGTTGGTACGCGT | 7152 |
| 288 | PLILSPS | 7153 | CCCTTGATCTTATCTCCAAGT | 7154 |
| 289 | SVNQAVT | 7155 | TCTGTGAATCAGGCGGTTACG | 7156 |
| 290 | AHGERLS | 7157 | GCTCACGGAGAAAGACTTAGC | 7158 |
| 291 | TLASSER | 7159 | ACTTTGGCGAGTTCTGAGCGG | 7160 |
| 292 | VHDSTPL | 7161 | GTGCATGATTCGACTCCGTTG | 7162 |
| 293 | HAAGASS | 7163 | CATGCGGCGGGTGCTAGTAGT | 7164 |
| 294 | GNGTGVL | 7165 | GGAAACGGCACCGGGGTCCTA | 7166 |
| 295 | VLTSPGP | 7167 | GTGCTCACAAGCCCGGGACCG | 7168 |
| 296 | LSTGAQM | 7169 | TTATCAACCGGAGCTCAAATG | 7170 |
| 297 | MIASGLS | 7171 | ATGATTGCGTCGGGTTTGTCG | 7172 |
| 298 | RYDVEST | 7173 | CGGTATGATGTTGAGTCTACG | 7174 |
| 299 | VLVGTSL | 7175 | GTTCTTGTTGGGACGAGTTTG | 7176 |
| 300 | LNTTESK | 7177 | CTTAACACCACCGAAAGCAAA | 7178 |
| 301 | SNIPTLM | 7179 | TCGAACATCCCGACATTAATG | 7180 |
| 302 | VLGGPAV | 7181 | GTGCTGGGTGGTCCTGCGGTG | 7182 |
| 303 | VIGGLGI | 7183 | GTTATTGGTGGGCTTGGGATT | 7184 |
| 304 | DLASAGH | 7185 | GATCTGGCGAGTGCTGGGCAT | 7186 |
| 305 | SVTDIKH | 7187 | TCGGTGACGGACATAAAACAC | 7188 |
| 306 | TNHQEPN | 7189 | ACGAATCATCAGGAGCCTAAT | 7190 |
| 307 | VLNEHVA | 7191 | GTCCTTAACGAACACGTAGCT | 7192 |
| 308 | HNTGMDM | 7193 | CATAATACGGGGATGGATATG | 7194 |
| 309 | GRSQLPM | 7195 | GGCCGATCACAACTTCCAATG | 7196 |
| 310 | DSGKDRT | 7197 | GATTCTGGTAAGGATCGTACG | 7198 |
| 311 | LLAGISI | 7199 | TTGCTTGCTGGGATTAGTATT | 7200 |
| 312 | TDVVLHK | 7201 | ACTGACGTCGTATTACACAAA | 7202 |
| 313 | VIETRLS | 7203 | GTCATCGAAACTCGCCTTTCC | 7204 |
| 314 | FGAELHK | 7205 | TTTGGGGCTGAGTTGCATAAG | 7206 |
| 315 | SHGTDSK | 7207 | AGTCACGGCACGGACTCTAAA | 7208 |
| 316 | AVDSSVR | 7209 | GCTGTTGACTCCAGCGTTAGA | 7210 |
| 317 | PNQGTLR | 7211 | CCTAATCAGGGGACGCTTCGG | 7212 |
| 318 | VVSVTAS | 7213 | GTGGTGTCGGTTACGGCTAGT | 7214 |
| 319 | VNVSYGD | 7215 | GTGAATGTTTCGTATGGTGAT | 7216 |
| 320 | VTTVYPV | 7217 | GTTACGACAGTATACCCGGTA | 7218 |
| 321 | QYVVSGA | 7219 | CAGTATGTTGTTAGTGGTGCG | 7220 |
| 322 | VLSGEVL | 7221 | GTCTTGTCTGGAGAAGTCCTT | 7222 |
| 323 | AAGVILN | 7223 | GCGGCGGGTGTTATTCTGAAT | 7224 |
| 324 | SMTSESS | 7225 | TCAATGACTTCGGAATCGTCT | 7226 |
| 325 | ILVDTHA | 7227 | ATTCTGGTTGATACTCATGCG | 7228 |
| 326 | YVTFGEN | 7229 | TACGTAACCTTCGGTGAAAAC | 7230 |
| 327 | NSDLMGR | 7231 | AACAGTGACCTAATGGGCCGA | 7232 |
| 328 | IVDYQGK | 7233 | ATCGTAGACTACCAAGGCAAA | 7234 |
| 329 | PHQGSES | 7235 | CCTCATCAGGGTAGTGAGAGT | 7236 |
| 330 | LSRGEEK | 7237 | CTTTCGAGGGGTGAGGAGAAG | 7238 |
| 331 | LSRDVAV | 7239 | TTGTCGAGGGATGTGGCGGTT | 7240 |
| 332 | PTQGTLR | 7241 | CCAACGCAAGGTACCTTGCGA | 7242 |
| 333 | REQQKYW | 7243 | CGGGAACAACAAAAATACTGG | 7244 |
| 334 | EQSMGSP | 7245 | GAGCAGTCTATGGGTTCTCCG | 7246 |
| 335 | KGSETPM | 7247 | AAAGGGTCAGAAACACCGATG | 7248 |
| 336 | KEYITAV | 7249 | AAAGAATACATAACAGCGGTA | 7250 |
| 337 | HGTLESQ | 7251 | CACGGCACCCTCGAATCGCAA | 7252 |
| 338 | ESLAGVR | 7253 | GAGAGTCTTGCTGGTGTTAGG | 7254 |
| 339 | NDRNTSS | 7255 | AATGATAGGAATACGTCTTCG | 7256 |
| 340 | AAVSALL | 7257 | GCCGCAGTATCCGCACTATTA | 7258 |
| 341 | LRVTENP | 7259 | CTTCGGGTCACCGAAAACCCC | 7260 |
| 342 | IAILAAS | 7261 | ATTGCGATTCTTGCTGCTTCG | 7262 |
| 343 | MLTGIAT | 7263 | ATGTTGACGGGGATTGCTACT | 7264 |
| 344 | STIPALM | 7265 | AGTACTATTCCTGCTCTGATG | 7266 |
| 345 | AAREVIN | 7267 | GCGGCTCGGGAGGTGATTAAT | 7268 |
| 346 | IVMAEVH | 7269 | ATCGTAATGGCCGAAGTACAC | 7270 |
| 347 | LVVDASR | 7271 | TTGGTAGTAGACGCAAGTCGC | 7272 |
| 348 | HQHMVEG | 7273 | CACCAACACATGGTTGAAGGA | 7274 |
| 349 | ENSGGHF | 7275 | GAGAATAGTGGGGGTCATTTT | 7276 |
| 350 | YSMTVTT | 7277 | TATAGTATGACGGTTACGACT | 7278 |
| 351 | SHASDSK | 7279 | TCGCACGCATCAGACTCTAAA | 7280 |
| 352 | AVSDYTV | 7281 | GCCGTGAGCGACTACACAGTC | 7282 |
| 353 | STIPTLL | 7283 | AGTACTATTCCTACTCTGTTG | 7284 |
| 354 | SPSAFPK | 7285 | TCCCCTTCAGCATTCCCAAAA | 7286 |
| 355 | NFGEVQL | 7287 | AATTTTGGTGAGGTTCAGCTG | 7288 |
| 356 | GIETRGL | 7289 | GGAATCGAAACACGCGGTCTC | 7290 |
| 357 | NVNQDSL | 7291 | AACGTAAACCAAGACTCACTC | 7292 |
| 358 | AGLLTKV | 7293 | GCAGGACTCCTTACAAAAGTA | 7294 |
| 359 | LSIRQGP | 7295 | TTGAGTATTCGTCAGGGTCCT | 7296 |
| 360 | PALQGNF | 7297 | CCGGCTCTTCAGGGTAATTTT | 7298 |
| 361 | LDSGIPR | 7299 | CTCGACTCTGGTATCCCCAGA | 7300 |
| 362 | SYSDGSS | 7301 | TCATACTCGGACGGCAGCAGC | 7302 |
| 363 | FQDTIGV | 7303 | TTTCAGGATACGATTGGGGTG | 7304 |
| 364 | VLLGIDR | 7305 | GTTTTGCTAGGAATCGACCGT | 7306 |
| 365 | SHGYDSK | 7307 | TCTCATGGTTATGATTCGAAG | 7308 |
| 366 | SVDTGLL | 7309 | AGCGTCGACACGGGCCTCTTA | 7310 |
| 367 | EGGGAQR | 7311 | GAGGGGGGTGGGGCTCAGAGG | 7312 |
| 368 | AALSQEF | 7313 | GCGGCCCTGTCTCAAGAATTC | 7314 |
| 369 | NSISLIN | 7315 | AACTCTATCAGCCTCATAAAC | 7316 |
| 370 | LSRGEEM | 7317 | CTTTCGAGGGGTGAGGAGATG | 7318 |
| 371 | IGMSAST | 7319 | ATAGGTATGAGCGCGTCCACC | 7320 |
| 372 | MGSDTTM | 7321 | ATGGGTTCTGATACTACTATG | 7322 |
| 373 | SLVLTSH | 7323 | TCATTAGTCCTTACGAGCCAC | 7324 |
| 374 | LGGDAVA | 7325 | CTAGGAGGAGACGCAGTTGCA | 7326 |
| 375 | PIQGTLR | 7327 | CCTATTCAGGGGACGCTTCGG | 7328 |
| 376 | RVELALT | 7329 | AGAGTCGAACTTGCCTTAACA | 7330 |
| 377 | VDHGGVH | 7331 | GTTGACCACGGAGGGGTCCAC | 7332 |
| 378 | IASDIGR | 7333 | ATTGCTTCGGATATTGGTCGG | 7334 |
| 379 | PNERLAV | 7335 | CCTAACGAACGATTGGCAGTC | 7336 |
| 380 | DLSTFPV | 7337 | GACCTCTCGACATTCCCTGTA | 7338 |
| 381 | DSSKAEW | 7339 | GATAGTAGTAAGGCTGAGTGG | 7340 |
| 382 | GAFAPAT | 7341 | GGCGCATTCGCACCAGCAACA | 7342 |
| 383 | TMSLSLR | 7343 | ACTATGTCTCTGTCGTTGCGT | 7344 |
| 384 | VDDIKSW | 7345 | GTTGACGACATAAAATCCTGG | 7346 |
| 385 | TTLADHA | 7347 | ACTACTCTGGCTGATCATGCG | 7348 |
| 386 | KSDVEYL | 7349 | AAATCAGACGTCGAATACCTA | 7350 |
| 387 | MNGGYVL | 7351 | ATGAACGGCGGATACGTACTT | 7352 |
| 388 | MAVDVTK | 7353 | ATGGCAGTCGACGTAACCAAA | 7354 |
| 389 | TDALTSK | 7355 | ACAGACGCACTCACCAGTAAA | 7356 |
| 390 | AAGGILN | 7357 | GCGGCGGGTGGTATTCTGAAT | 7358 |
| 391 | LSEGRAY | 7359 | CTTAGTGAGGGTCGTGCGTAT | 7360 |
| 392 | SVSHVVV | 7361 | TCGGTCTCTCACGTCGTCGTA | 7362 |
| 393 | FISGALT | 7363 | TTCATATCCGGCGCCTTAACT | 7364 |
| 394 | VTGLTVQ | 7365 | GTTACCGGGCTGACAGTACAA | 7366 |
| 395 | HSASLIE | 7367 | CACTCAGCATCCCTCATAGAA | 7368 |
| 396 | ITQAVYI | 7369 | ATCACACAAGCGGTATACATC | 7370 |
| 397 | ASMSAEH | 7371 | GCTAGTATGTCTGCGGAGCAT | 7372 |
| 398 | SVTDVNH | 7373 | TCTGTTACTGATGTTAATCAT | 7374 |
| 399 | LGTSDVR | 7375 | TTGGGGACGAGTGATGTGCGT | 7376 |
| 400 | QSNHAPV | 7377 | CAATCAAACCACGCCCCGGTC | 7378 |
| 401 | SMAVTAK | 7379 | AGTATGGCGGTGACGGCGAAG | 7380 |
| 402 | RMTGDLT | 7381 | CGTATGACTGGAGACCTAACC | 7382 |
| 403 | SHGSDPK | 7383 | AGCCACGGGTCAGACCCTAAA | 7384 |
| 404 | GLGDSGE | 7385 | GGGTTGGGGGATTCGGGTGAG | 7386 |
| 405 | SVTLLGV | 7387 | AGTGTGACTCTGTTGGGTGTG | 7388 |
| 406 | PNDGPSK | 7389 | CCTAATGATGGGCCTAGTAAG | 7390 |
| 407 | FDSAPRY | 7391 | TTTGATTCTGCGCCGCGGTAT | 7392 |
| 408 | VVDAYNL | 7393 | GTCGTAGACGCTTACAACTTA | 7394 |
| 409 | NEAVNVR | 7395 | AATGAGGCTGTTAATGTTCGG | 7396 |
| 410 | TLALSER | 7397 | ACCTTAGCCTTATCAGAACGA | 7398 |
| 411 | LRDSAEP | 7399 | CTCCGAGACTCAGCGGAACCA | 7400 |
| 412 | PDNNPRN | 7401 | CCTGATAATAATCCGCGGAAT | 7402 |
| 413 | YHASDSK | 7403 | TATCATGCTTCTGATTCGAAG | 7404 |
| 414 | KGYDTNM | 7405 | AAAGGCTACGACACAAACATG | 7406 |
| 415 | HTTGAEM | 7407 | CACACTACTGGGGCCGAAATG | 7408 |
| 416 | GLNDNVA | 7409 | GGTCTGAATGATAATGTGGCG | 7410 |
| 417 | PQLIVPK | 7411 | CCTCAGCTTATTGTTCCTAAG | 7412 |
| 418 | IIVDNGS | 7413 | ATAATAGTCGACAACGGATCA | 7414 |
| 419 | QNESGMK | 7415 | CAAAACGAAAGCGGGATGAAA | 7416 |
| 420 | NQLGELV | 7417 | AACCAACTCGGCGAACTAGTG | 7418 |
| 421 | RDLTSDM | 7419 | AGAGACTTGACTTCGGACATG | 7420 |
| 422 | GVSVLNV | 7421 | GGGGTGAGTGTGCTGAATGTT | 7422 |
| 423 | AADSSGR | 7423 | GCGGCGGATAGTTCTGGGCGG | 7424 |
| 424 | ALNEHEA | 7425 | GCTCTGAATGAGCATGAGGCG | 7426 |
| 425 | LRVTENQ | 7427 | TTGCGTGTGACGGAGAATCAG | 7428 |
| 426 | MTVPGSP | 7429 | ATGACGGTTCCGGGTAGTCCG | 7430 |
| 427 | TLAITER | 7431 | ACTTTGGCGATTACTGAGCGG | 7432 |
| 428 | KNPGVDT | 7433 | AAGAATCCGGGGGTGGATACT | 7434 |
| 429 | RVALDET | 7435 | AGGGTGGCGCTGGATGAGACG | 7436 |
| 430 | MNVGHVL | 7437 | ATGAATGTGGGTCATGTTCTG | 7438 |
| 431 | LRVTENK | 7439 | TTGCGTGTGACGGAGAATAAG | 7440 |
| 432 | TLGMSTR | 7441 | ACGTTGGGGATGTCTACTCGT | 7442 |
| 433 | REHSAQL | 7443 | AGGGAGCATTCGGCGCAGCTT | 7444 |
| 434 | HGNLVSQ | 7445 | CATGGGAATTTGGTGTCTCAG | 7446 |
| 435 | VQGPQTG | 7447 | GTGCAGGGTCCGCAGACTGGT | 7448 |
| 436 | VTTLTPV | 7449 | GTGACTACGCTTACTCCTGTG | 7450 |
| 437 | DSHVSGM | 7451 | GATAGTCATGTGTCGGGGATG | 7452 |
| 438 | LVTPMHM | 7453 | CTCGTAACTCCCATGCACATG | 7454 |
| 439 | RVDSEKL | 7455 | AGGGTGGATTCGGAGAAGCTT | 7456 |
| 440 | RPEIEVR | 7457 | CGGCCGGAGATTGAGGTTAGG | 7458 |
| 441 | QDGPAEK | 7459 | CAGGATGGGCCTGCGGAGAAG | 7460 |
| 442 | MVTPTNT | 7461 | ATGGTTACTCCTACGAATACG | 7462 |
| 443 | LSKGSQL | 7463 | CTGTCTAAGGGGTCGCAGCTG | 7464 |
| 444 | VIVLTEA | 7465 | GTGATTGTGTTGACGGAGGCT | 7466 |
| 445 | QSLTDGV | 7467 | CAGAGTTTGACTGATGGGGTT | 7468 |
| 446 | MNGAHVL | 7469 | ATGAATGGGGCTCATGTTCTG | 7470 |
| 447 | RVALDLT | 7471 | AGGGTGGCGCTGGATTTGACG | 7472 |
| 448 | SQSAFPN | 7473 | AGTCAGTCGGCTTTTCCTAAT | 7474 |
| 449 | LTRGEEK | 7475 | CTTACGAGGGGTGAGGAGAAG | 7476 |
| 450 | MGASDTL | 7477 | ATGGGGGCTAGTGATACGCTT | 7478 |
| 451 | NQLAELV | 7479 | AATCAGTTGGCGGAGCTGGTT | 7480 |
| 452 | LGDSADQ | 7481 | CTTGGGGATTCTGCTGATCAG | 7482 |
| 453 | GVSVLND | 7483 | GGGGTGAGTGTGCTGAATGAT | 7484 |
| 454 | TTAAIVK | 7485 | ACGACGGCGGCTATTGTTAAG | 7486 |
| 455 | MGASDTH | 7487 | ATGGGGGCTAGTGATACGCAT | 7488 |
| 456 | DLNEHVA | 7489 | GATCTGAATGAGCATGTGGCG | 7490 |
| 457 | MNGGHAL | 7491 | ATGAATGGGGGTCATGCTCTG | 7492 |
| 458 | SNGLPAQ | 7493 | AGTAATGGGCTTCCTGCGCAG | 7494 |
| 459 | TTTGNLM | 7495 | ACGACGACGGGGAATCTTATG | 7496 |
| 460 | LAGSTGP | 7497 | TTGGCGGGGTCGACGGGTCCG | 7498 |
| 461 | AVKEYEL | 7499 | GCCGTTAAAGAATACGAACTC | 7500 |
| 462 | VIAGHGN | 7501 | GTTATTGCTGGGCATGGGAAT | 7502 |
| 463 | LGDSAET | 7503 | CTTGGGGATTCTGCTGAGACG | 7504 |
| 464 | MVTPTNK | 7505 | ATGGTTACTCCTACGAATAAG | 7506 |
| 465 | AIVSIAR | 7507 | GCGATTGTGTCGATTGCTCGG | 7508 |
| 466 | SPTSSPT | 7509 | TCTCCGACGAGTTCGCCGACT | 7510 |
| 467 | ESRNDVV | 7511 | GAGTCGAGGAATGATGTTGTT | 7512 |
| 468 | TGSSAML | 7513 | ACGGGGAGTTCGGCGATGCTT | 7514 |
| 469 | TVNSIPV | 7515 | ACGGTCAACAGTATACCAGTC | 7516 |
| 470 | ITENASR | 7517 | ATTACTGAGAATGCGTCGCGG | 7518 |
| 471 | PILGAST | 7519 | CCGATTCTTGGTGCTAGTACG | 7520 |
| 472 | GGKGEGP | 7521 | GGTGGGAAGGGTGAGGGTCCG | 7522 |
| 473 | IVMDENH | 7523 | ATCGTAATGGACGAAAACCAC | 7524 |
| 474 | MGASVTL | 7525 | ATGGGGGCTAGTGTTACGCTT | 7526 |
| 475 | AVKEYEA | 7527 | GCGGTGAAGGAGTATGAGGCG | 7528 |
| 476 | TVGLSIA | 7529 | ACTGTGGGTTTGTCGATTGCG | 7530 |
| 477 | QVIDTKT | 7531 | CAGGTGATTGATACTAAGACT | 7532 |
| 478 | DVVLLTR | 7533 | GATGTTGTTTTGTTGACTAGG | 7534 |
| 479 | DVRGSDI | 7535 | GACGTACGGGGGTCTGACATC | 7536 |
| 480 | FAEVAQA | 7537 | TTTGCGGAGGTTGCGCAGGCG | 7538 |
| 481 | TSLLPQT | 7539 | ACGTCTCTGCTTCCTCAGACT | 7540 |
| 482 | AADSSAR | 7541 | GCGGCGGATAGTTCTGCGCGG | 7542 |
| 483 | LGDSAES | 7543 | CTTGGGGATTCTGCTGAGTCG | 7544 |
| 484 | SVDSGLL | 7545 | AGTGTTGATAGTGGGCTGCTT | 7546 |
| 485 | GRDLTPA | 7547 | GGTCGGGATCTTACGCCTGCT | 7548 |
| 486 | PAREVLY | 7549 | CCGGCTCGGGAGGTGCTTTAT | 7550 |
| 487 | GSDIKHE | 7551 | GGTTCTGATATTAAGCATGAG | 7552 |
| 488 | LAGSPGP | 7553 | TTGGCGGGGTCGCCGGGTCCG | 7554 |
| 489 | DVTVSMR | 7555 | GATGTTACTGTTTCTATGCGT | 7556 |
| 490 | RVDSGQL | 7557 | AGGGTGGATTCGGGGCAGCTT | 7558 |
| 491 | MNGGNVM | 7559 | ATGAATGGGGGTAATGTTATG | 7560 |
| 492 | PQLIVPA | 7561 | CCTCAGCTTATTGTTCCTGCG | 7562 |
| 493 | VTTHTPV | 7563 | GTGACTACGCATACTCCTGTG | 7564 |
| 494 | MQITGLH | 7565 | ATGCAGATTACTGGTCTTCAT | 7566 |
| 495 | AAREELN | 7567 | GCGGCTCGGGAGGAGCTTAAT | 7568 |
| 496 | AAREVLM | 7569 | GCGGCTCGGGAGGTGCTTATG | 7570 |
| 497 | PGGHYQA | 7571 | CCGGGTGGGCATTATCAGGCT | 7572 |
| 498 | EVAGTYS | 7573 | GAGGTGGCTGGGACGTATTCT | 7574 |
| 499 | VVDSNNL | 7575 | GTTGTTGATTCGAATAATCTG | 7576 |
| 500 | VRQLDSL | 7577 | GTGAGGCAGCTGGATTCGCTG | 7578 |
| 501 | QVTDTKT | 7579 | CAGGTGACTGATACTAAGACT | 7580 |
| 502 | VNDGLGI | 7581 | GTTAATGATGGGCTTGGGATT | 7582 |
| 503 | TTSANLM | 7583 | ACGACGTCGGCGAATCTTATG | 7584 |
| 504 | GSHVSGD | 7585 | GGTAGTCATGTGTCGGGGGAT | 7586 |
| 505 | GRSQLPM | 7587 | GGGCGGTCGCAGTTGCCGATG | 7588 |
| 506 | TVLAASH | 7589 | ACGGTGTTGGCTGCGTCTCAT | 7590 |
| 507 | SPSAFPN | 7591 | AGTCCGTCGGCTTTTCCTAAT | 7592 |
| 508 | QSMTDGV | 7593 | CAGAGTATGACTGATGGGGTT | 7594 |
| 509 | QSLSKDK | 7595 | CAGAGTCTTAGTAAGGATAAG | 7596 |
| 510 | REALSVT | 7597 | AGGGAGGCGCTGTCTGTGACG | 7598 |
| 511 | VIAGHGI | 7599 | GTTATTGCTGGGCATGGGATT | 7600 |
| 512 | MNGGHVI | 7601 | ATGAATGGGGGTCATGTTATT | 7602 |
| 513 | NQLAEQV | 7603 | AATCAGTTGGCGGAGCAGGTT | 7604 |
| 514 | PAREVHY | 7605 | CCGGCTCGGGAGGTGCATTAT | 7606 |
| 515 | VLASLGP | 7607 | GTTCTGGCTTCGCTTGGTCCT | 7608 |
| 516 | PARELHY | 7609 | CCGGCTCGGGAGCTGCATTAT | 7610 |
| 517 | MSITEPR | 7611 | ATGTCTATTACTGAGCCGCGG | 7612 |
| 518 | AAGVIPN | 7613 | GCGGCGGGTGTTATTCCGAAT | 7614 |
| 519 | VTRGTGN | 7615 | GTTACTCGTGGTACGGGTAAT | 7616 |
| 520 | GVGVLNV | 7617 | GGGGTGGGTGTGCTGAATGTT | 7618 |
| 521 | QSPGSQL | 7619 | CAGTCTCCGGGGTCGCAGCTG | 7620 |
| 522 | RPEIAGR | 7621 | CGGCCGGAGATTGCGGGTAGG | 7622 |
| 523 | PILGASS | 7623 | CCGATTCTTGGTGCTAGTTCG | 7624 |
| 524 | RVDSEQL | 7625 | AGGGTGGATTCGGAGCAGCTT | 7626 |
| 525 | TTYDTLV | 7627 | ACGACGTATGATACGTTGGTT | 7628 |
| 526 | VFVEKSA | 7629 | GTTTTTGTTGAGAAGAGTGCG | 7630 |
| 527 | VGSLTAS | 7631 | GTGGGGTCGCTTACGGCTAGT | 7632 |
| 528 | QNMGVTQ | 7633 | CAGAATATGGGGGTGACTCAG | 7634 |
| 529 | GVSVPNV | 7635 | GGGGTGAGTGTGCCGAATGTT | 7636 |
| 530 | GSRENAR | 7637 | GGGAGTAGGGAGAATGCGCGT | 7638 |
| 531 | PGEHYEA | 7639 | CCGGGTGAGCATTATGAGGCT | 7640 |
| 532 | AVGVILN | 7641 | GCGGTGGGTGTTATTCTGAAT | 7642 |
| 533 | TLAINER | 7643 | ACTTTGGCGATTAATGAGCGG | 7644 |
| 534 | MNGGHVL | 7645 | ATGAATGGGGGTCATGTTCTG | 7646 |
| 535 | LGGVSSE | 7647 | CTGGGGGGTGTGTCGTCTGAG | 7648 |
| 536 | TVGLTIA | 7649 | ACTGTGGGTTTGACGATTGCG | 7650 |
| 537 | WNGRETT | 7651 | TGGAATGGTCGGGAGACTACT | 7652 |
| 538 | RHVHVEG | 7653 | CGCCACGTACACGTCGAAGGC | 7654 |
| 539 | DSRVSGD | 7655 | GATAGTCGTGTGTCGGGGGAT | 7656 |
| 540 | RPEIAVR | 7657 | CGGCCGGAGATTGCGGTTAGG | 7658 |
| 541 | DVSVSMR | 7659 | GATGTTTCTGTTTCTATGCGT | 7660 |
| 542 | IVTPTNT | 7661 | ATTGTTACTCCTACGAATACG | 7662 |
| 543 | SPTSSPP | 7663 | TCTCCGACGAGTTCGCCGCCT | 7664 |
| 544 | NHGTDSK | 7665 | AACCACGGAACAGACTCTAAA | 7666 |
| 545 | KNPGVDS | 7667 | AAGAATCCGGGGGTGGATTCT | 7668 |
| 546 | PDRHGGL | 7669 | CCTGATCGGCATGGTGGGCTG | 7670 |
| 547 | VVDSDNL | 7671 | GTTGTTGATTCGGATAATCTG | 7672 |
| 548 | NQLGELV | 7673 | AATCAGTTGGGGGAGCTGGTT | 7674 |
| 549 | NQLAEPV | 7675 | AATCAGTTGGCGGAGCCGGTT | 7676 |
| 550 | WIGRETT | 7677 | TGGATTGGTCGGGAGACTACT | 7678 |
| 551 | SGAPLRL | 7679 | TCTGGGGCGCCGCTTAGGCTT | 7680 |
| 552 | VLLGINM | 7681 | GTGCTTTTGGGTATTAATATG | 7682 |
| 553 | SHGYDSK | 7683 | TCGCACGGCTACGACTCTAAA | 7684 |
| 554 | PGAHYQA | 7685 | CCGGGTGCGCATTATCAGGCT | 7686 |
| 555 | ASESSPP | 7687 | GCATCAGAATCATCACCACCC | 7688 |
| 556 | QNVGVTQ | 7689 | CAGAATGTGGGGGTGACTCAG | 7690 |
| 557 | GEQQKVW | 7691 | GGTGAGCAGCAGAAGGTTTGG | 7692 |
| 558 | AQAQTGW | 7693 | GCTCAAGCACAGACCGGCTGG | 7694 |
| 559 | STLHTTT | 7695 | AGTACTCTTCATACTACGACT | 7696 |
| 560 | AVLSQNL | 7697 | GCTGTGTTGTCTCAGAATCTT | 7698 |
| 561 | GAVSSTK | 7699 | GGTGCTGTTTCTTCGACTAAG | 7700 |
| 562 | PTQETLR | 7701 | CCTACTCAGGAGACGCTTCGG | 7702 |
| 563 | QYVVSGV | 7703 | CAGTATGTTGTTAGTGGTGTG | 7704 |
| 564 | LAGLGGP | 7705 | CTTGCGGGTTTGGGGGGGCCT | 7706 |
| 565 | QTMKDFY | 7707 | CAGACGATGAAGGATTTTTAT | 7708 |
| 566 | VGSVMAS | 7709 | GTGGGGTCGGTTATGGCTAGT | 7710 |
| 567 | AHIGTLT | 7711 | GCACACATCGGAACTCTCACC | 7712 |
| 568 | MVTPTIT | 7713 | ATGGTTACTCCTACGATTACG | 7714 |
| 569 | MLTPTNT | 7715 | ATGCTTACTCCTACGAATACG | 7716 |
| 570 | TGDRDQN | 7717 | ACTGGTGATCGGGATCAGAAT | 7718 |
| 571 | GGVSSTN | 7719 | GGTGGTGTTTCTTCGACTAAT | 7720 |
| 572 | LSNHGPI | 7721 | CTGAGTAATCATGGGCCTATT | 7722 |
| 573 | ALTNGQR | 7723 | GCACTAACCAACGGTCAACGT | 7724 |
| 574 | NQLSELV | 7725 | AATCAGTTGTCGGAGCTGGTT | 7726 |
| 575 | GALTSTK | 7727 | GGTGCTCTTACTTCGACTAAG | 7728 |
| 576 | VGSVTAS | 7729 | GTGGGGTCGGTTACGGCTAGT | 7730 |
| 577 | VLASHGT | 7731 | GTTCTGGCTTCGCATGGTACT | 7732 |
| 578 | AVKEYET | 7733 | GCGGTGAAGGAGTATGAGACG | 7734 |
| 579 | RGGVSTE | 7735 | CGGGGGGGTGTGTCGACTGAG | 7736 |
| 580 | SGGKEEM | 7737 | AGTGGGGGTAAGGAGGAGATG | 7738 |
| 581 | HGTLVSQ | 7739 | CATGGGACTTTGGTGTCTCAG | 7740 |
| 582 | LMNDLLS | 7741 | CTTATGAACGACTTACTCTCC | 7742 |
| 583 | DAPRDGA | 7743 | GACGCACCCCGCGACGGGGCT | 7744 |
| 584 | RTTEPRF | 7745 | CGTACTACGGAGCCTCGTTTT | 7746 |
| 585 | TLPELNL | 7747 | ACGTTGCCGGAGTTGAATCTT | 7748 |
| 586 | LTKSTEW | 7749 | CTCACCAAATCCACAGAATGG | 7750 |
| 587 | QVPDNKT | 7751 | CAGGTGCCTGATAATAAGACT | 7752 |
| 588 | QGGDSGG | 7753 | CAGGGTGGTGATAGTGGGGGT | 7754 |
| 589 | LSTGEEM | 7755 | CTTTCGACGGGTGAGGAGATG | 7756 |
| 590 | PEPRSSY | 7757 | CCTGAGCCGCGTAGTAGTTAT | 7758 |
| 591 | LISTTLR | 7759 | TTGATTTCTACTACGCTGCGT | 7760 |
| 592 | RVTPTNT | 7761 | CGCGTGACGCCAACTAACACT | 7762 |
| 593 | HKDRTTL | 7763 | CATAAGGATAGGACGACGCTT | 7764 |
| 594 | STEYAML | 7765 | TCTACTGAGTATGCGATGTTG | 7766 |
| 595 | NLGAELS | 7767 | AACTTGGGGGCAGAACTATCG | 7768 |
| 596 | QNGLQLL | 7769 | CAGAATGGGTTGCAGCTTTTG | 7770 |
| 597 | LISGTLR | 7771 | TTGATTTCTGGTACGCTGCGT | 7772 |
| 598 | ANQNVII | 7773 | GCAAACCAAAACGTAATAATA | 7774 |
| 599 | SPPPNAR | 7775 | AGCCCGCCGCCGAACGCGCGT | 7776 |
| 600 | SSADYQV | 7777 | AGTTCTGCGGATTATCAGGTT | 7778 |
| 601 | KQVSMES | 7779 | AAGCAGGTGTCGATGGAGTCG | 7780 |
| 602 | RVALDVT | 7781 | AGGGTGGCGCTGGATGTGACG | 7782 |
| 603 | LNMGPLH | 7783 | CTGAATATGGGTCCTTTGCAT | 7784 |
| 604 | IPRIHSL | 7785 | ATTCCTCGGATTCATTCTCTT | 7786 |
| 605 | IGSSLSP | 7787 | ATTGGGTCGTCGCTTAGTCCT | 7788 |
| 606 | LEKDPMT | 7789 | TTGGAAAAAGACCCTATGACT | 7790 |
| 607 | MVTNTNT | 7791 | ATGGTTACTAATACGAATACG | 7792 |
| 608 | RIASNLA | 7793 | AGGATTGCTTCTAATCTGGCG | 7794 |
| 609 | VVAGTNS | 7795 | GTCGTTGCAGGTACAAACTCG | 7796 |
| 610 | GVAATNS | 7797 | GGGGTGGCTGCGACGAATTCT | 7798 |
| 611 | KGSVTPM | 7799 | AAGGGTTCTGTTACTCCTATG | 7800 |
| 612 | HDTSASV | 7801 | CATGATACTAGTGCTAGTGTT | 7802 |
| 613 | SLAITER | 7803 | AGTTTGGCGATTACTGAGCGG | 7804 |
| 614 | HGRDALV | 7805 | CATGGGCGGGATGCTCTTGTG | 7806 |
| 615 | DIAGLGI | 7807 | GATATTGCCGGGCTTGGGATT | 7808 |
| 616 | ANQLAPV | 7809 | GCCAACCAATTGGCCCCCGTG | 7810 |
| 617 | NGASLAS | 7811 | AACGGAGCTTCCCTCGCAAGC | 7812 |
| 618 | *KMSAYV | 7813 | TGAAAGATGTCCGCTTATGTG | 7814 |
| 619 | GVAGRIL | 7815 | GGGGTGGCTGGGCGTATTCTG | 7816 |
| 620 | TLAISGR | 7817 | ACTTTGGCGATTTCTGGGCGG | 7818 |
| 621 | NLHTAEA | 7819 | AACCTCCACACTGCTGAAGCG | 7820 |
| 622 | SIAVGLS | 7821 | AGTATTGCGGTGGGTTTGTCG | 7822 |
| 623 | TGQQVSI | 7823 | ACTGGGCAGCAGGTTAGTATT | 7824 |
| 624 | LPRLGGL | 7825 | CTTCCGCGTTTGGGGGGGCTT | 7826 |
| 625 | DTASTQS | 7827 | GACACAGCATCTACTCAATCC | 7828 |
| 626 | PQLIVPV | 7829 | CCTCAGCTTATTGTTCCTGTG | 7830 |
| 627 | GLNDHVA | 7831 | GGTCTGAATGATCATGTGGCG | 7832 |
| 628 | RDEAYRA | 7833 | AGGGATGAGGCTTATCGTGCG | 7834 |
| 629 | RISPEGT | 7835 | CGTATATCACCGGAAGGCACT | 7836 |
| 630 | HSEGVGR | 7837 | CATAGTGAGGGTGTTGGGCGG | 7838 |
| 631 | KGSDNTM | 7839 | AAGGGTTCTGATAATACTATG | 7840 |
| 632 | LPNGGGF | 7841 | CTGCCGAATGGGGGGGGGTTT | 7842 |
| 633 | AVTNPLM | 7843 | GCGGTTACTAATCCTTTGATG | 7844 |
| 634 | VTVAGSV | 7845 | GTTACGGTGGCTGGTTCGGTG | 7846 |
| 635 | RDDQGIP | 7847 | CGGGATGATCAGGGGATTCCG | 7848 |
| 636 | HTLSTGV | 7849 | CACACCCTAAGCACGGGAGTA | 7850 |
| 637 | SGGTRGP | 7851 | TCTGGTGGGACTCGTGGTCCT | 7852 |
| 638 | RHIADAS | 7853 | AGACACATAGCGGACGCGTCG | 7854 |
| 639 | SGISFLA | 7855 | AGCGGAATCAGCTTCTTGGCT | 7856 |
| 640 | SALTQGY | 7857 | TCGGCGCTAACCCAAGGATAC | 7858 |
| 641 | LNGAPLL | 7859 | CTGAATGGTGCGCCGTTGCTG | 7860 |
| 642 | STVGINV | 7861 | AGTACGGTCGGGATCAACGTT | 7862 |
| 643 | QEQGTTT | 7863 | CAGGAGCAGGGTACGACTACT | 7864 |
| 644 | MIGGHVQ | 7865 | ATGATTGGGGGTCATGTTCAG | 7866 |
| 645 | RVLTSDV | 7867 | CGTGTTCTGACGTCTGATGTG | 7868 |
| 646 | GFGLTED | 7869 | GGGTTTGGGTTGACGGAGGAT | 7870 |
| 647 | DAQSRLA | 7871 | GATGCTCAGTCGCGGTTGGCG | 7872 |
| 648 | RESANAD | 7873 | CGTGAGTCTGCGAATGCTGAT | 7874 |
| 649 | LLHGIIA | 7875 | CTTTTACACGGAATAATCGCC | 7876 |
| 650 | GMGASSK | 7877 | GGTATGGGGGCGTCTTCTAAG | 7878 |
| 651 | RNEGINQ | 7879 | CGTAATGAGGGTATTAATCAG | 7880 |
| 652 | PGVAMVT | 7881 | CCCGGGGTCGCTATGGTAACT | 7882 |
| 653 | ASQLTQT | 7883 | GCGTCTCAGCTTACTCAGACT | 7884 |
| 654 | ALGDQAR | 7885 | GCGTTAGGGGACCAAGCGCGT | 7886 |
| 655 | LTDVTQM | 7887 | TTAACCGACGTCACACAAATG | 7888 |
| 656 | DVAISMR | 7889 | GACGTAGCGATATCCATGCGA | 7890 |
| 657 | YGSNVLS | 7891 | TACGGTTCTAACGTCCTCTCA | 7892 |
| 658 | VYHGGVD | 7893 | GTGTATCATGGTGGTGTGGAT | 7894 |
| 659 | SFDTYGA | 7895 | TCCTTCGACACTTACGGGGCC | 7896 |
| 660 | PTTNPLL | 7897 | CCGACTACTAATCCGCTTCTG | 7898 |
| 661 | RVAMSVT | 7899 | AGGGTGGCGATGTCTGTGACG | 7900 |
| 662 | HIVLSHA | 7901 | CATATTGTGCTGAGTCATGCT | 7902 |
| 663 | TLQELQL | 7903 | ACGTTGCAGGAGTTGCAGCTT | 7904 |
| 664 | DPSLGSP | 7905 | GATCCGTCTCTGGGTTCTCCG | 7906 |
| 665 | LAGSVVV | 7907 | CTGGCGGGTTCGGTTGTTGTG | 7908 |
| 666 | ILVDAYA | 7909 | ATACTAGTAGACGCGTACGCT | 7910 |
| 667 | GVANVSP | 7911 | GGAGTTGCTAACGTCAGCCCA | 7912 |
| 668 | RMTLTGD | 7913 | CGTATGACTTTGACTGGTGAT | 7914 |
| 669 | SGGVESK | 7915 | TCTGGTGGTGTTGAGTCGAAG | 7916 |
| 670 | QIHDTAL | 7917 | CAAATCCACGACACAGCGCTC | 7918 |
| 671 | FQVEQIM | 7919 | TTTCAGGTTGAGCAGATTATG | 7920 |
| 672 | GLVQMSS | 7921 | GGTCTGGTGCAGATGTCTTCT | 7922 |
| 673 | FPSMSGK | 7923 | TTCCCAAGCATGTCGGGGAAA | 7924 |
| 674 | VSNGHFV | 7925 | GTTAGTAATGGGCATTTTGTT | 7926 |
| 675 | STVGSSP | 7927 | AGTACGGTGGGGTCGTCGCCG | 7928 |
| 676 | YLVTADN | 7929 | TATTTGGTTACTGCTGATAAT | 7930 |
| 677 | TTRADPA | 7931 | ACTACTCGGGCTGATCCTGCG | 7932 |
| 678 | PLVPQGG | 7933 | CCCTTAGTACCTCAAGGCGGT | 7934 |
| 679 | GARMVMT | 7935 | GGTGCGCGGATGGTTATGACT | 7936 |
| 680 | MKTQIEL | 7937 | ATGAAAACGCAAATAGAACTC | 7938 |
| 681 | VNHGGVD | 7939 | GTAAACCACGGAGGAGTTGAC | 7940 |
| 682 | MVQSGLT | 7941 | ATGGTTCAGTCGGGGTTGACG | 7942 |
| 683 | QYAVSGG | 7943 | CAATACGCAGTGAGCGGCGGT | 7944 |
| 684 | MTSGNLM | 7945 | ATGACCTCTGGCAACCTCATG | 7946 |
| 685 | TTLAHPA | 7947 | ACTACTCTGGCTCATCCTGCG | 7948 |
| 686 | REQQKAW | 7949 | CGAGAACAACAAAAAGCCTGG | 7950 |
| 687 | VTTLSPV | 7951 | GTGACTACGCTTTCTCCTGTG | 7952 |
| 688 | LQDRTTL | 7953 | CTCCAAGACCGCACTACTCTC | 7954 |
| 689 | SLGALVA | 7955 | TCGCTGGGTGCTCTGGTTGCT | 7956 |
| 690 | GADDAAL | 7957 | GGAGCCGACGACGCAGCCCTC | 7958 |
| 691 | IGPRREV | 7959 | ATAGGACCTCGCCGTGAAGTA | 7960 |
| 692 | QSQTAVA | 7961 | CAGTCTCAGACGGCTGTTGCT | 7962 |
| 693 | VDFGDHT | 7963 | GTAGACTTCGGCGACCACACC | 7964 |
| 694 | SLRDTHY | 7965 | AGTCTTCGGGATACTCATTAT | 7966 |
| 695 | YEHSGLL | 7967 | TATGAGCATTCTGGTCTTTTG | 7968 |
| 696 | VTELTRF | 7969 | GTGACTGAGCTTACGCGGTTT | 7970 |
| 697 | LTHLRVS | 7971 | CTGACTCACCTTCGTGTCAGC | 7972 |
| 698 | QRSDSVM | 7973 | CAGCGGTCGGATAGTGTGATG | 7974 |
| 699 | LSKEHAP | 7975 | TTGAGTAAGGAGCATGCTCCT | 7976 |
| 700 | PDGAAPM | 7977 | CCTGATGGTGCGGCTCCTATG | 7978 |
| 701 | LTTPIEL | 7979 | CTAACTACCCCTATAGAACTC | 7980 |
| 702 | AAVVPRY | 7981 | GCAGCAGTAGTACCACGATAC | 7982 |
| 703 | VVLSLAT | 7983 | GTTGTCTTAAGTCTAGCCACT | 7984 |
| 704 | QDAHVAI | 7985 | CAGGATGCGCATGTGGCTATT | 7986 |
| 705 | LGHANGL | 7987 | TTAGGGCACGCAAACGGACTT | 7988 |
| 706 | SPQGVLA | 7989 | TCGCCGCAGGGGGTTCTTGCT | 7990 |
| 707 | LSLTMPA | 7991 | CTCTCGCTTACAATGCCTGCC | 7992 |
| 708 | YVGSPLV | 7993 | TATGTTGGTTCTCCGTTGGTG | 7994 |
| 709 | QILGASS | 7995 | CAAATCTTAGGGGCCTCGAGT | 7996 |
| 710 | NSGSMHT | 7997 | AACTCAGGAAGCATGCACACT | 7998 |
| 711 | GVLGQTD | 7999 | GGTGTGTTGGGGCAGACTGAT | 8000 |
| 712 | MNVGHVL | 8001 | ATGAACGTAGGGCACGTCCTC | 8002 |
| 713 | PNTRDPI | 8003 | CCTAATACGCGGGATCCGATT | 8004 |
| 714 | VEKRHMV | 8005 | GTGGAGAAGAGGCATATGGTG | 8006 |
| 715 | VVSGIPN | 8007 | GTGGTGTCTGGTATTCCGAAT | 8008 |
| 716 | KNGGHDL | 8009 | AAAAACGGTGGGCACGACCTA | 8010 |
| 717 | YESTRGQ | 8011 | TATGAGTCGACGAGGGGTCAG | 8012 |
| 718 | NALGDGY | 8013 | AACGCGCTGGGCGACGGCTAC | 8014 |
| 719 | GLYDAAT | 8015 | GGGCTTTATGATGCGGCGACT | 8016 |
| 720 | LVAGQAM | 8017 | CTGGTGGCGGGGCAGGCTATG | 8018 |
| 721 | DSRTVDS | 8019 | GACTCTCGAACCGTCGACTCA | 8020 |
| 722 | GDRGVVA | 8021 | GGTGATAGGGGGGTTGTGGCT | 8022 |
| 723 | GLESSVP | 8023 | GGCCTTGAAAGCTCTGTACCC | 8024 |
| 724 | LSRGAEN | 8025 | CTTTCGAGGGGTGCGGAGAAT | 8026 |
| 725 | ISMTLLP | 8027 | ATTTCGATGACTCTGCTGCCG | 8028 |
| 726 | AVGNVLL | 8029 | GCTGTGGGGAATGTGCTTTTG | 8030 |
| 727 | QYAVSGG | 8031 | CAGTATGCTGTTAGTGGTGGG | 8032 |
| 728 | PAQGTLR | 8033 | CCTGCTCAGGGGACGCTTCGG | 8034 |
| 729 | DVAVYIR | 8035 | GATGTTGCTGTTTATATTCGT | 8036 |
| 730 | VLQLAAL | 8037 | GTTCTTCAACTCGCTGCCCTC | 8038 |
| 731 | DDAVSKR | 8039 | GATGATGCTGTTTCTAAGCGT | 8040 |
| 732 | LEDRSAS | 8041 | TTGGAGGATCGGTCGGCTAGT | 8042 |
| 733 | PSYQGNG | 8043 | CCGAGTTATCAGGGGAATGGT | 8044 |
| 734 | LGDSDET | 8045 | TTAGGAGACTCGGACGAAACC | 8046 |
| 735 | GNLLLTA | 8047 | GGTAATTTGCTGCTTACTGCT | 8048 |
| 736 | EGVSALL | 8049 | GAGGGTGTTTCTGCGTTGTTG | 8050 |
| 737 | GHQNGGI | 8051 | GGGCACCAAAACGGCGGAATC | 8052 |
| 738 | RSISGDW | 8053 | CGTTCCATAAGTGGCGACTGG | 8054 |
| 739 | YLALTGI | 8055 | TATCTTGCGCTTACGGGGATT | 8056 |
| 740 | LSDGGPL | 8057 | CTCTCGGACGGAGGCCCCCTC | 8058 |
| 741 | LEANVSH | 8059 | CTTGAGGCGAATGTTTCGCAT | 8060 |
| 742 | GLSERAQ | 8061 | GGCCTGTCCGAACGAGCACAA | 8062 |
| 743 | SGFVVPV | 8063 | TCTGGGTTTGTTGTGCCGGTG | 8064 |
| 744 | GVMLLTE | 8065 | GGGGTTATGTTGCTGACTGAG | 8066 |
| 745 | STTSSPS | 8067 | TCGACCACCTCATCCCCTAGC | 8068 |
| 746 | FNGLPAQ | 8069 | TTCAACGGTCTCCCCGCACAA | 8070 |
| 747 | HVSGASL | 8071 | CACGTGTCCGGCGCCAGCTTA | 8072 |
| 748 | GGDTSRS | 8073 | GGGGGTGATACGAGTCGTAGT | 8074 |
| 749 | AVAGTNS | 8075 | GCAGTTGCGGGTACAAACTCG | 8076 |
| 750 | VMSGTSH | 8077 | GTTATGTCGGGTACTAGTCAT | 8078 |
| 751 | YAGIAQG | 8079 | TATGCGGGGATTGCTCAGGGT | 8080 |
| 752 | MLALAVT | 8081 | ATGTTGGCGCTGGCTGTGACG | 8082 |
| 753 | AALTREI | 8083 | GCTGCTCTTACGCGGGAGATT | 8084 |
| 754 | AIVGMLS | 8085 | GCGATTGTGGGTATGCTGTCG | 8086 |
| 755 | MANMLSV | 8087 | ATGGCGAACATGTTATCGGTG | 8088 |
| 756 | LLADERV | 8089 | TTACTCGCAGACGAAAGGGTC | 8090 |
| 757 | LSSTDGV | 8091 | CTGAGTTCGACTGATGGGGTT | 8092 |
| 758 | VTQNLSE | 8093 | GTGACGCAGAATTTGAGTGAG | 8094 |
| 759 | PARYRLW | 8095 | CCGGCGCGGTATCGGCTTTGG | 8096 |
| 760 | GGDALNQ | 8097 | GGGGGGGACGCCCTTAACCAA | 8098 |
| 761 | VMASPGP | 8099 | GTTATGGCTTCGCCTGGTCCT | 8100 |
| 762 | PGDRDQY | 8101 | CCAGGCGACCGAGACCAATAC | 8102 |
| 763 | LGSLVVH | 8103 | CTGGGAAGCTTAGTCGTTCAC | 8104 |
| 764 | LEVGALR | 8105 | CTGGAAGTAGGCGCACTTCGT | 8106 |
| 765 | VSPSVLQ | 8107 | GTTAGTCCTTCGGTGCTTCAG | 8108 |
| 766 | GISGEVS | 8109 | GGTATTTCGGGGGAGGTGAGT | 8110 |
| 767 | RGAEVLL | 8111 | CGGGGTGCGGAGGTGCTGCTG | 8112 |
| 768 | GVAGTNS | 8113 | GGAGTTGCGGGAACAAACTCC | 8114 |
| 769 | LNGGIGV | 8115 | CTTAATGGGGGTATTGGGGTT | 8116 |
| 770 | TIAAHVP | 8117 | ACCATAGCAGCCCACGTACCC | 8118 |
| 771 | LNGISFV | 8119 | TTGAATGGGATTTCGTTTGTG | 8120 |
| 772 | MGVGGGS | 8121 | ATGGGGGTCGGTGGTGGATCC | 8122 |
| 773 | PLKGGGE | 8123 | CCGTTGAAAGGCGGGGGTGAA | 8124 |
| 774 | RVAQALT | 8125 | AGGGTGGCGCAGGCTCTGACG | 8126 |
| 775 | EASSRLL | 8127 | GAAGCTTCGTCGCGACTTCTC | 8128 |
| 776 | SQAEGSV | 8129 | TCCCAAGCGGAAGGCAGCGTG | 8130 |
| 777 | NSGPQLS | 8131 | AACTCGGGCCCACAACTTTCG | 8132 |
| 778 | VQSADPR | 8133 | GTCCAATCCGCGGACCCTCGC | 8134 |
| 779 | VSDSSIN | 8135 | GTGTCGGATTCGTCTATTAAT | 8136 |
| 780 | TVKEYEL | 8137 | ACCGTTAAAGAATACGAACTC | 8138 |
| 781 | MENAPGR | 8139 | ATGGAGAATGCTCCTGGGAGG | 8140 |
| 782 | GNGDMFA | 8141 | GGGAATGGGGATATGTTTGCT | 8142 |
| 783 | HTSGTSS | 8143 | CATACGAGTGGGACGTCGTCG | 8144 |
| 784 | VIASNEP | 8145 | GTTATAGCCTCCAACGAACCG | 8146 |
| 785 | GINEHVA | 8147 | GGGATCAACGAACACGTAGCC | 8148 |
| 786 | HNSHVLT | 8149 | CACAACTCCCACGTATTAACC | 8150 |
| 787 | QANMLTV | 8151 | CAGGCTAATATGTTGACTGTT | 8152 |
| 788 | VFTGTDP | 8153 | GTGTTCACCGGCACAGACCCT | 8154 |
| 789 | ASDAVLR | 8155 | GCATCCGACGCCGTCCTAAGG | 8156 |
| 790 | ASDAVLR | 8157 | GCTAGTGATGCGGTGTTGCGT | 8158 |
| 791 | RDLTNDV | 8159 | CGCGACTTAACTAACGACGTT | 8160 |
| 792 | RVHSAQL | 8161 | AGGGTGCATTCGGCGCAGCTT | 8162 |
| 793 | SGNAWDE | 8163 | AGTGGGAATGCTTGGGATGAG | 8164 |
| 794 | GHQALNA | 8165 | GGCCACCAAGCATTAAACGCC | 8166 |
| 795 | IADMGGN | 8167 | ATTGCTGATATGGGTGGTAAT | 8168 |
| 796 | SMDSTSR | 8169 | TCTATGGATTCGACGTCTAGG | 8170 |
| 797 | GVSLPMS | 8171 | GGCGTATCACTACCCATGAGC | 8172 |
| 798 | AALAGSR | 8173 | GCGGCTCTGGCGGGGTCTAGG | 8174 |
| 799 | ILGVYSD | 8175 | ATACTGGGCGTTTACTCCGAC | 8176 |
| 800 | APRDPGV | 8177 | GCGCCGCGTGATCCTGGTGTT | 8178 |
| 801 | NRHETLS | 8179 | AACCGCCACGAAACACTATCA | 8180 |
| 802 | LGDGTTR | 8181 | CTGGGGGATGGTACGACTCGG | 8182 |
| 803 | RNHDQTH | 8183 | AGAAACCACGACCAAACACAC | 8184 |
| 804 | MTDSGTV | 8185 | ATGACTGATAGTGGGACTGTG | 8186 |
| 805 | NHHGDRL | 8187 | AACCACCACGGAGACAGGCTG | 8188 |
| 806 | LANTVVT | 8189 | CTTGCTAATACGGTTGTGACG | 8190 |
| 807 | QFHENIR | 8191 | CAGTTTCATGAGAATATTCGT | 8192 |
| 808 | NFGRDTL | 8193 | AATTTTGGTCGTGATACTCTG | 8194 |
| 809 | SGSNTGP | 8195 | AGCGGCTCCAACACTGGCCCG | 8196 |
| 810 | EPAMGMR | 8197 | GAGCCGGCGATGGGGATGAGG | 8198 |
| 811 | ENAGTDV | 8199 | GAAAACGCCGGAACTGACGTC | 8200 |
| 812 | IIISSAN | 8201 | ATAATCATATCCTCGGCCAAC | 8202 |
| 813 | NHVGDRL | 8203 | AATCATGTTGGTGATCGTTTG | 8204 |
| 814 | SGGLMTG | 8205 | AGTGGTGGTCTTATGACTGGT | 8206 |
| 815 | GRGTNDH | 8207 | GGTCGGGGTACGAATGATCAT | 8208 |
| 816 | LANMLQV | 8209 | TTGGCAAACATGCTTCAAGTG | 8210 |
| 817 | TNTDSSL | 8211 | ACGAATACGGATTCTAGTCTG | 8212 |
| 818 | GSGPGVA | 8213 | GGTTCTGGGCCGGGGGTGGCT | 8214 |
| 819 | ADVLIRG | 8215 | GCGGACGTGCTCATACGCGGT | 8216 |
| 820 | TLQQLQL | 8217 | ACTCTCCAACAACTGCAATTG | 8218 |
| 821 | TMANSER | 8219 | ACGATGGCAAACTCGGAACGC | 8220 |
| 822 | WDDQTSG | 8221 | TGGGATGATCAGACTTCGGGG | 8222 |
| 823 | GTGSTNV | 8223 | GGAACTGGATCGACAAACGTT | 8224 |
| 824 | GPSGAGI | 8225 | GGGCCATCAGGGGCAGGCATC | 8226 |
| 825 | NAAVIYD | 8227 | AACGCTGCAGTGATATACGAC | 8228 |
| 826 | SNLGETV | 8229 | TCGAATTTGGGGGAGACGGTT | 8230 |
| 827 | EPSLGSR | 8231 | GAGCCGTCTCTGGGTTCTCGG | 8232 |
| 828 | IGASVKL | 8233 | ATCGGTGCATCGGTAAAACTG | 8234 |
| 829 | SRGVISS | 8235 | AGCCGAGGCGTAATCTCGTCA | 8236 |
| 830 | RVMGEEV | 8237 | CGTGTGATGGGGGAGGAGGTT | 8238 |
| 831 | YSTERSV | 8239 | TATTCGACTGAGAGGTCTGTT | 8240 |
| 832 | AGGGTPR | 8241 | GCGGGGGGTGGGACTCCGAGG | 8242 |
| 833 | VLPSPGP | 8243 | GTTCTGCCTTCGCCTGGTCCT | 8244 |
| 834 | TSVLPQT | 8245 | ACGTCTGTGCTTCCTCAGACT | 8246 |
| 835 | ILASPGP | 8247 | ATACTTGCGTCACCCGGACCG | 8248 |
| 836 | GEIDIAF | 8249 | GGAGAAATCGACATAGCCTTC | 8250 |
| 837 | GWADSVP | 8251 | GGTTGGGCTGATTCGGTTCCG | 8252 |
| 838 | GVAATNT | 8253 | GGAGTTGCAGCCACAAACACG | 8254 |
| 839 | LVGNPST | 8255 | CTCGTGGGCAACCCGAGTACG | 8256 |
| 840 | YGVTLST | 8257 | TACGGCGTAACCCTCTCTACC | 8258 |
| 841 | ASMGTVA | 8259 | GCGTCCATGGGAACCGTAGCC | 8260 |
| 842 | WSNSEQH | 8261 | TGGTCGAATTCGGAGCAGCAT | 8262 |
| 843 | REVSPLM | 8263 | CGAGAAGTAAGCCCCCTGATG | 8264 |
| 844 | QAESAAR | 8265 | CAAGCGGAATCAGCGGCTAGA | 8266 |
| 845 | ALQSAQV | 8267 | GCACTACAATCTGCACAAGTT | 8268 |
| 846 | PNDRLTV | 8269 | CCAAACGACCGGTTGACGGTT | 8270 |
| 847 | LIVTENQ | 8271 | TTGATTGTGACGGAGAATCAG | 8272 |
| 848 | GLVHMPS | 8273 | GGCTTAGTTCACATGCCCTCA | 8274 |
| 849 | MADGASM | 8275 | ATGGCGGATGGTGCGTCTATG | 8276 |
| 850 | RAVENMG | 8277 | CGCGCAGTAGAAAACATGGGC | 8278 |
| 851 | LNGVTIT | 8279 | CTCAACGGCGTCACCATCACC | 8280 |
| 852 | RYNVETA | 8281 | CGGTATAATGTTGAGACTGCG | 8282 |
| 853 | SLLHDGA | 8283 | AGTTTGTTGCATGATGGGGCG | 8284 |
| 854 | TRIGLSD | 8285 | ACACGAATAGGACTCAGTGAC | 8286 |
| 855 | NAHALMV | 8287 | AACGCCCACGCACTCATGGTC | 8288 |
| 856 | VEVQAGK | 8289 | GTGGAGGTTCAGGCTGGGAAG | 8290 |
| 857 | RGGVLSE | 8291 | CGAGGTGGGGTACTCAGTGAA | 8292 |
| 858 | KNQDTKM | 8293 | AAGAATCAGGATACGAAGATG | 8294 |
| 859 | QLRPLQT | 8295 | CAACTGCGTCCTTTGCAAACG | 8296 |
| 860 | LLENARV | 8297 | CTGCTGGAGAATGCGAGGGTG | 8298 |
| 861 | LFGPSAY | 8299 | TTATTCGGACCTTCCGCCTAC | 8300 |
| 862 | RIDAELL | 8301 | CGTATTGATGCTGAGTTGTTG | 8302 |
| 863 | VVSGLLH | 8303 | GTTGTCTCCGGGTTGCTACAC | 8304 |
| 864 | MGGVTSV | 8305 | ATGGGGGGGGTTACTTCGGTG | 8306 |
| 865 | TVADPRA | 8307 | ACTGTTGCGGATCCGCGGGCG | 8308 |
| 866 | TGLQVST | 8309 | ACTGGGCTGCAGGTTAGTACT | 8310 |
| 867 | ANEHNIA | 8311 | GCTAATGAGCATAATATTGCG | 8312 |
| 868 | STLASPR | 8313 | TCAACCCTAGCCTCGCCTCGA | 8314 |
| 869 | IHFSGDN | 8315 | ATCCACTTCAGCGGCGACAAC | 8316 |
| 870 | GLVQIVA | 8317 | GGGCTTGTTCAGATTGTTGCG | 8318 |
| 871 | TAYDTLV | 8319 | ACGGCGTATGATACGTTGGTT | 8320 |
| 872 | AVKEYQS | 8321 | GCTGTTAAAGAATACCAATCT | 8322 |
| 873 | ASSHVTV | 8323 | GCTTCGAGTCATGTTACTGTG | 8324 |
| 874 | STLSTFD | 8325 | TCGACTTTGAGTACGTTTGAT | 8326 |
| 875 | LDLTSDV | 8327 | CTTGATCTGACGTCTGATGTG | 8328 |
| 876 | QYNVEST | 8329 | CAGTATAATGTTGAGTCTACG | 8330 |
| 877 | SVEPLSL | 8331 | TCCGTAGAACCTCTATCCCTC | 8332 |
| 878 | PGHGPVR | 8333 | CCCGGGCACGGACCTGTACGC | 8334 |
| 879 | VRQLDSR | 8335 | GTGAGGCAGCTGGATTCGCGG | 8336 |
| 880 | MMLNQGS | 8337 | ATGATGCTTAACCAAGGCAGC | 8338 |
| 881 | EPSLSSP | 8339 | GAGCCGTCTCTGAGTTCTCCG | 8340 |
| 882 | PGVDTGV | 8341 | CCTGGTGTTGATACTGGTGTT | 8342 |
| 883 | SGDVARH | 8343 | TCAGGCGACGTTGCCCGACAC | 8344 |
| 884 | ADYGTSS | 8345 | GCGGACTACGGTACCAGCTCT | 8346 |
| 885 | VHSQDVS | 8347 | GTGCATTCGCAGGATGTGTCT | 8348 |
| 886 | VIAGLGV | 8349 | GTGATCGCGGGACTCGGCGTC | 8350 |
| 887 | VHVDNSN | 8351 | GTGCATGTTGATAATAGTAAT | 8352 |
| 888 | QSGVF*C | 8353 | CAGTCGGGGGTGTTCTGATGC | 8354 |
| 889 | AQDHGTL | 8355 | GCGCAGGATCATGGGACGTTG | 8356 |
| 890 | SRLEYIG | 8357 | AGCCGCCTTGAATACATCGGG | 8358 |
| 891 | VLLGINT | 8359 | GTCCTGCTCGGAATAAACACC | 8360 |
| 892 | LGIGQGP | 8361 | TTGGGTATTGGTCAGGGTCCT | 8362 |
| 893 | NVTATLG | 8363 | AACGTCACAGCAACGCTGGGT | 8364 |
| 894 | EVLSLAP | 8365 | GAGGTGCTGTCTCTTGCTCCG | 8366 |
| 895 | TNGVLYT | 8367 | ACAAACGGCGTCCTTTACACG | 8368 |
| 896 | RFVGSVP | 8369 | AGGTTTGTGGGTAGTGTTCCG | 8370 |
| 897 | TNGYRED | 8371 | ACTAATGGTTATAGGGAGGAT | 8372 |
| 898 | LESAAMI | 8373 | CTGGAGTCGGCTGCTATGATT | 8374 |
| 899 | VPLPSGK | 8375 | GTTCCTCTGCCGAGTGGGAAG | 8376 |
| 900 | NSKDVQR | 8377 | AACTCCAAAGACGTACAAAGA | 8378 |
| 901 | GVGGTYS | 8379 | GGAGTTGGGGGCACATACAGT | 8380 |
| 902 | LTDKMTS | 8381 | TTGACTGATAAGATGACGTCG | 8382 |
| 903 | SGAAAAT | 8383 | AGCGGGGCCGCAGCCGCCACC | 8384 |
| 904 | MVTTTNT | 8385 | ATGGTGACGACCACAAACACC | 8386 |
| 905 | TSLGLMQ | 8387 | ACTAGCCTTGGCTTAATGCAA | 8388 |
| 906 | LVHLGTS | 8389 | TTGGTTCATCTTGGGACTTCT | 8390 |
| 907 | NGMGDVT | 8391 | AATGGGATGGGTGATGTGACG | 8392 |
| 908 | LNSPLHV | 8393 | CTGAATAGTCCGCTGCATGTT | 8394 |
| 909 | GSRESVR | 8395 | GGGAGTAGGGAGAGTGTGCGT | 8396 |
| 910 | DNSPMDL | 8397 | GACAACAGCCCCATGGACCTA | 8398 |
| 911 | VVSPQPV | 8399 | GTGGTTTCGCCTCAACCGGTG | 8400 |
| 912 | STINTLM | 8401 | AGTACTATTAATACTCTGATG | 8402 |
| 913 | THGDAGG | 8403 | ACTCATGGGGATGCTGGTGGG | 8404 |
| 914 | AVLAGSS | 8405 | GCGGTTCTGGCGGGGTCTAGT | 8406 |
| 915 | YTSGTGT | 8407 | TACACCTCGGGCACAGGGACA | 8408 |
| 916 | GPDTGAM | 8409 | GGCCCCGACACAGGCGCGATG | 8410 |
| 917 | SGMQAEA | 8411 | TCGGGTATGCAGGCGGAGGCT | 8412 |
| 918 | LATHDAR | 8413 | CTCGCAACGCACGACGCACGA | 8414 |
| 919 | YDRIMSS | 8415 | TACGACCGCATAATGTCATCT | 8416 |
| 920 | RHHGTES | 8417 | CGTCATCATGGTACTGAGAGT | 8418 |
| 921 | MAVKSPP | 8419 | ATGGCTGTGAAGTCGCCGCCG | 8420 |
| 922 | EVRDTKT | 8421 | GAAGTTCGGGACACAAAAACG | 8422 |
| 923 | GFVQSRM | 8423 | GGGTTTGTTCAGAGTCGGATG | 8424 |
| 924 | VLAAVDR | 8425 | GTCCTTGCTGCCGTCGACCGA | 8426 |
| 925 | VTTVPPV | 8427 | GTGACTACGGTTCCTCCTGTG | 8428 |
| 926 | HFSSETS | 8429 | CACTTCTCTTCCGAAACTTCT | 8430 |
| 927 | TTVTVSL | 8431 | ACGACGGTGACGGTGTCGTTG | 8432 |
| 928 | AESRLFV | 8433 | GCGGAGAGTAGGCTGTTTGTG | 8434 |
| 929 | LSGGFTA | 8435 | TTGAGTGGTGGTTTTACGGCG | 8436 |
| 930 | NSDLASP | 8437 | AATAGTGATTTGGCGTCTCCT | 8438 |
| 931 | LDHGASA | 8439 | TTAGACCACGGAGCGTCGGCG | 8440 |
| 932 | YGSNDLS | 8441 | TATGGGAGTAATGATCTGAGT | 8442 |
| 933 | VIASNEH | 8443 | GTCATAGCCTCAAACGAACAC | 8444 |
| 934 | LTGSIGL | 8445 | TTAACTGGGTCAATTGGACTC | 8446 |
| 935 | HLSRDHS | 8447 | CACCTGTCACGTGACCACTCA | 8448 |
| 936 | NLRGEHT | 8449 | AATTTGCGTGGGGAGCATACG | 8450 |
| 937 | ILVDALA | 8451 | ATTCTGGTTGATGCTCTTGCG | 8452 |
| 938 | SGYDTSV | 8453 | AGTGGGTATGATACGTCGGTT | 8454 |
| 939 | HKDKWVG | 8455 | CACAAAGACAAATGGGTTGGG | 8456 |
| 940 | MTGNSFV | 8457 | ATGACAGGCAACAGCTTCGTA | 8458 |
| 941 | YTVGSLA | 8459 | TACACCGTTGGCTCACTCGCC | 8460 |
| 942 | SVSKPFL | 8461 | AGTGTGAGTAAGCCTTTTTTG | 8462 |
| 943 | TVMTSEP | 8463 | ACAGTTATGACCAGCGAACCT | 8464 |
| 944 | SMGYVSA | 8465 | TCGATGGGTTATGTTTCGGCT | 8466 |
| 945 | ILVDAYA | 8467 | ATTCTGGTTGATGCTTATGCG | 8468 |
| 946 | QGGTTLR | 8469 | CAAGGGGGGACTACTCTACGC | 8470 |
| 947 | SEGLSRD | 8471 | TCGGAGGGTCTTTCGCGTGAT | 8472 |
| 948 | FTGGTGT | 8473 | TTTACTGGTGGTACGGGTACT | 8474 |
| 949 | RSGSGVA | 8475 | CGGTCGGGCTCCGGAGTCGCC | 8476 |
| 950 | VLASLGP | 8477 | GTGCTCGCCAGTCTCGGCCCC | 8478 |
| 951 | LVTGMSS | 8479 | CTTGTCACGGGCATGTCAAGC | 8480 |
| 952 | ALASTQT | 8481 | GCACTAGCATCGACCCAAACT | 8482 |
| 953 | SLVRGLL | 8483 | AGTCTTGTTCGGGGTTTGCTG | 8484 |
| 954 | VGQVPGR | 8485 | GTGGGGCAAGTCCCGGGTAGG | 8486 |
| 955 | NGPMKAD | 8487 | AACGGTCCAATGAAAGCAGAC | 8488 |
| 956 | GPMASVV | 8489 | GGGCCGATGGCGTCTGTGGTT | 8490 |
| 957 | LVSGLGP | 8491 | CTTGTGAGTGGGCTGGGTCCG | 8492 |
| 958 | AADRSVR | 8493 | GCAGCAGACCGCTCCGTACGT | 8494 |
| 959 | AATSGGP | 8495 | GCAGCCACCAGTGGCGGGCCG | 8496 |
| 960 | RDLTSNV | 8497 | CGAGACTTAACTAGCAACGTA | 8498 |
| 961 | IVMSSHI | 8499 | ATCGTCATGAGCTCCCACATC | 8500 |
| 962 | GPLNQSL | 8501 | GGTCCGCTGAATCAGTCTTTG | 8502 |
| 963 | TDGRTLH | 8503 | ACGGATGGTAGGACGCTGCAT | 8504 |
| 964 | TGLQVSF | 8505 | ACTGGGCTGCAGGTTAGTTTT | 8506 |
| 965 | RVTTHTP | 8507 | CGTGTTACTACTCATACGCCG | 8508 |
| 966 | EVGSIGS | 8509 | GAGGTTGGTAGTATTGGTTCT | 8510 |
| 967 | TDVHSTS | 8511 | ACGGATGTGCATTCGACTTCG | 8512 |
| 968 | TFAISDR | 8513 | ACTTTTGCGATTTCTGATCGG | 8514 |
| 969 | TVLAAAH | 8515 | ACGGTGTTGGCTGCGGCTCAT | 8516 |
| 970 | MNDAGRD | 8517 | ATGAATGATGCTGGGCGTGAT | 8518 |
| 971 | PAEHYQA | 8519 | CCGGCTGAGCATTATCAGGCT | 8520 |
| 972 | DRSTAEW | 8521 | GACCGCTCCACAGCAGAATGG | 8522 |
| 973 | LYGGSSA | 8523 | CTCTACGGAGGGTCCTCGGCT | 8524 |
| 974 | VTQAVYV | 8525 | GTTACGCAGGCTGTTTATGTT | 8526 |
| 975 | GVNHAVA | 8527 | GGAGTCAACCACGCCGTCGCC | 8528 |
| 976 | DSAPAAR | 8529 | GATTCGGCTCCGGCGGCTCGG | 8530 |
| 977 | DPKTGWR | 8531 | GATCCGAAGACTGGGTGGCGT | 8532 |
| 978 | SIVGSVQ | 8533 | TCAATCGTAGGCTCAGTCCAA | 8534 |
| 979 | DSDSGRR | 8535 | GATTCTGATAGTGGGCGGCGG | 8536 |
| 980 | EQYLGSP | 8537 | GAGCAGTATCTGGGTTCTCCG | 8538 |
| 981 | LSLDRPS | 8539 | CTAAGTCTAGACCGACCCTCG | 8540 |
| 982 | MGDIVTL | 8541 | ATGGGGGATATTGTTACGCTT | 8542 |
| 983 | SFRDTVP | 8543 | AGTTTTAGGGATACGGTGCCT | 8544 |
| 984 | RGLSDPV | 8545 | CGGGGGCTGTCTGATCCGGTG | 8546 |
| 985 | TGGLLYS | 8547 | ACTGGTGGGCTTCTTTATAGT | 8548 |
| 986 | VVLSGIS | 8549 | GTGGTTTTGTCGGGGATTTCT | 8550 |
| 987 | GVAGTYL | 8551 | GGGGTGGCTGGGACGTATCTG | 8552 |
| 988 | LNGSHGP | 8553 | CTGAATGGGTCGCATGGGCCG | 8554 |
| 989 | PSGALMT | 8555 | CCTTCAGGCGCCTTGATGACG | 8556 |
| 990 | LSLTDGV | 8557 | TTGTCCTTAACCGACGGAGTG | 8558 |
| 991 | GRDLTPA | 8559 | GGGCGTGACCTGACTCCAGCG | 8560 |
| 992 | AGHSNAV | 8561 | GCTGGGCATTCTAATGCGGTT | 8562 |
| 993 | GVAGTDS | 8563 | GGGGTGGCTGGGACGGATTCT | 8564 |
| 994 | AELGIRY | 8565 | GCTGAGCTGGGGATTAGGTAT | 8566 |
| 995 | PLSNAAL | 8567 | CCTCTATCTAACGCAGCACTG | 8568 |
| 996 | IGLSVST | 8569 | ATTGGGCTGTCTGTTTCTACT | 8570 |
| 997 | RSITIGP | 8571 | CGTTCGATTACTATTGGGCCG | 8572 |
| 998 | GLVRIQD | 8573 | GGACTGGTTCGGATCCAAGAC | 8574 |
| 999 | LSGIMVS | 8575 | TTGTCGGGGATTATGGTTTCG | 8576 |
| 1000 | SWQSDTD | 8577 | TCGTGGCAGTCTGATACGGAT | 8578 |

Table 9. PAL2 and AAV9 transgene expression and vector genome abundance in one cynomolgus macaque. ^{a}Transgene mRNA expression normalized to expression of GAPDH mRNA as detected by qPCR with a standard curve ^{b}Vector DNA normalized to the number of GAPDH genomic DNA copies as detected by qPCR with a standard curve

TABLE 9

| | PAL2 | AAV9 | PAL2/AV9 | PAL2 vector | AAV9 vector | PAL2/AAV9 |
|---|---|---|---|---|---|---|
| Tissue | mRNA* | mRNA* | mRNA | genomes/cell^{b} | genomes/cell^{b} | vector DNA |
| Frontal lobe | 3.81E−05 | 7.82E−06 | 4.87 | 6.25E−3 | 4.86E−03 | 1.29 |
| Temporal lobe | 4.8E−05 | 1.10E−05 | 4.37 | 1.13E−02 | 1.09E−02 | 1.04 |
| (anterior) | | | | | | |
| Temporal lobe | 6.27E−05 | 1.17E−05 | 5.35 | 1.01E−02 | 8.07E−03 | 1.25 |
| (posterior) | | | | | | |
| Parietal lobe | 7.98E−05 | 1.35E−05 | 5.93 | 1.10E−02 | 9.11E−03 | .21 |
| (anterior) | | | | | | |
| Parietal lobe | 1.19E−04 | 2.28E−05 | 5.23 | 1.37E−02 | 1.13E−02 | 1.22 |
| (posterior) | | | | | | |
| Occipital lobe | 1.06E−04 | 1.93E−05 | 5.50 | 6.73E−03 | 4.83E−03 | 1.39 |
| Thalamus | 5.54E−05 | 1.20E−05 | 4.61 | 2.04E−02 | 7.96E−03 | 2.56 |
| Midbrain | 9.67E−05 | 1.97E−05 | 4.90 | 9.28E−03 | 5.01E−03 | 1.85 |
| Corpus | 8.27E−05 | 3.03E−05 | 2.73 | 3.67E−03 | 1.93E−03 | 1.91 |
| callosum | | | | | | |
| Cerebellum | 6.69E−05 | 3.16E−05 | 2.11 | 2.18E−03 | 1.44E−03 | 1.51 |
| Neuroretina | 1.63E−04 | 1.22E−05 | 13.40 | 3.19E−03 | 8.52E−04 | 3.75 |
| RPE | 4.01E−04 | 1.72E−04 | 2.34 | 1.12E−01 | 1.85E−01 | 0.61 |
| Brain Stem | 9.34E−05 | 4.00E−05 | 2.33 | 8.97E−03 | 5.76E−03 | 1.56 |
| Cervical | 4.34E−04 | 1.44E−04 | 3.01 | 2.08E−02 | 1.42E−02 | 1.46 |
| spinal cord | | | | | | |
| Thoracic | 9.41E−04 | 3.19E−04 | 2.94 | 2.38E−02 | 1.84E−02 | 1.29 |
| spinal cord | | | | | | |
| Lumbar spinal | 1.71E−03 | 4.78E−04 | 3.58 | 3.15E−02 | 1.74E−02 | 1.80 |
| cord | | | | | | |
| Cauda equina | 3.90E−02 | 6.81E−03 | 5.74 | 6.35E−02 | 3.42E−02 | 1.86 |
| Cervical DRG | 3.12E−02 | 3.23E−03 | 9.64 | 5.08E−02 | 3.32E−02 | 1.53 |
| Thoracic DRG | 1.55E−02 | 1.97E−03 | 7.83 | 3.57E−02 | 1.93E−02 | 1.84 |
| Lumbar DRG | 3.98E−02 | 6.07E−03 | 6.56 | 1.74E−01 | 1.37E−01 | 1.27 |
| Triceps | 5.08E−02 | 1.72E−02 | 2.95 | 2.03E−01 | 2.49E−01 | 0.82 |
| Quadriceps | 5.37E−03 | 1.46E−03 | 3.68 | 2.70E−02 | 4.36E−02 | 0.62 |
| Diaphragm | 2.79E−02 | 8.01E−03 | 3.48 | 1.55E−01 | 1.97E−01 | 0.79 |
| Heart | 2.76E−02 | 1.15E−02 | 2.39 | 1.37E−01 | 2.04E−01 | 0.67 |
| Kidney | 4.81E−04 | 1.71E−04 | 2.81 | 1.53E−01 | 1.73E−01 | 0.88 |
| Lung | 8.12E−04 | 3.95E−04 | 2.06 | 3.44E−01 | 4.10E−01 | 0.84 |
| Thymus | 3.38E−03 | 1.85E−03 | 1.83 | 1.44E−02 | 4.69E−03 | 3.06 |
| Gonad | 3.20E−03 | 2.89E−03 | 1.11 | 1.83E−02 | 2.25E−02 | 0.81 |
| Liver | 8.35E−02 | 1.73E−01 | 0.48 | 1.31E+−01 | 5.02E+01 | 0.26 |
| Spleen | 1.84E−04 | 3.78E−04 | 0.49 | 6.29E+00 | 1.27E+00 | 4.97 |

### Benchmarking Engineered AAV Capsids in Mice and Macaques.

Applicant assessed the relative performance of mouse- and macaque-derived engineered variants in order to determine if any variants had strong neurotropic properties in both mice and macaques. Applicant performed a benchmarking experiment in C57BL/6J and BALB/cJ mice as well as in cynomolgus macaques comparing four mouse-derived variants and eight macaque-derived variants from this study with AAV9. This panel also included three promising engineered variants developed by the Gradinaru lab using the M-CREATE platform: PHP.C2, which is known to transduce the CNS of both C57BL/6J and BALB/cJ mice,²³ and AAV.CAP-B 10 and AAV.CAP-B22, two PHP.eB-derived variants that were initially selected in Cre-transgenic mice but have demonstrated enhanced neurotropic activity in marmosets” (FIG. 17A). Applicant generated rAAVs with each of these 16 capsids packaging a human frataxin (hFN) transgene—the gene involved in the degenerative neurological disorder Freidreich's ataxia—under control of the constitutive CBh promoter. The transgene of rAAVs produced with each capsid contained a unique set of fifty 20-mer barcodes in the 3′UTR region, which allowed us to associate sequenced hFXN transcripts with a specific capsid variant (FIG. 17B). We administered a pool containing equal proportions of each of these 16 capsid variants by intravenous (IV) injection to C57BL/6J and BALB/cJ mice and cynomolgus macaques at a total combined dose of 3E+13 vg/kg.

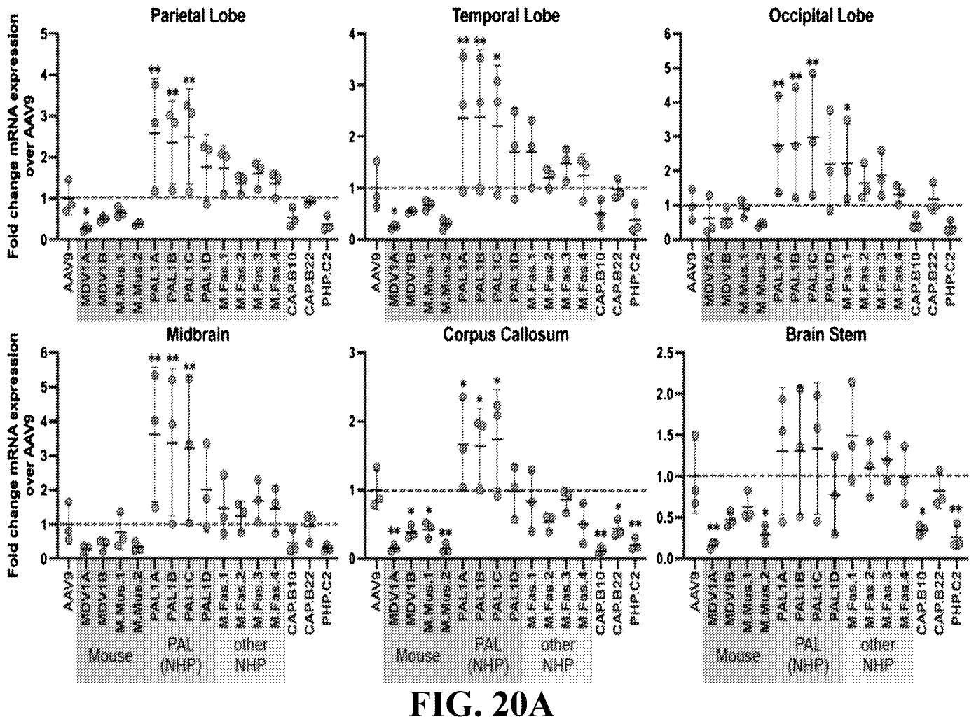

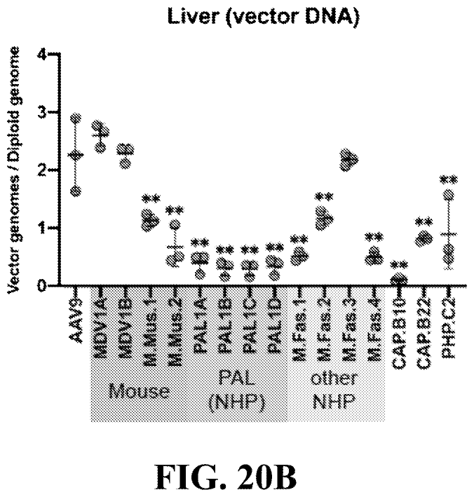

Applicant found that the efficacy of each variant tested was linked to the animal model in which it was initially identified and no variant exhibited cross-species CNS-tropic behavior. Quantification of hFXN mRNA expression revealed that none of the eight variants selected in macaques were capable of enhanced transduction of the brain or spinal cord of either mouse strain (FIG. 17C-17D). Likewise, none of the seven mouse-derived variants that we tested effectively transduced the macaque CNS (FIG. 4E). Remarkably, the AAV.CAP-B10 and AAV.CAP-B22 variants, which have previously been shown to outperform AAV9 in transducing the marmoset brain following systemic administration,²⁵ did not show increased performance in any area of the macaque CNS (FIGS. 4E and 20A).

Applicant were able to verify the efficacy of mouse-derived variants in the mouse strain in which each was initially discovered. All four mouse-derived variants identified in this study significantly outperformed AAV9 in transducing the brain and spinal cord of both mouse strains by a considerable margin, although of these four variants, only M.Mus.1 and M.Mus.2 were detargeted from the liver (FIG. 17C-17D). The AAV.CAP-B10, AAV.CAP-B22, and PHP.C2 variants performed exceptionally well in the C57BL/6J brain and spinal cord (FIG. 17C). PHP.C2 was also able to successfully transduce the BALB/cJ brain and spinal cord in line with previous findings²³ (FIG. 17D). However, as with the PHP.eB variant from which they were derived,^{27,28} the CNS tropism of AAV.CAP-B10 and AAV.CAP-B22 did not extend to BALB/cJ mice (FIG. 17D).

Applicant found that a number of variants discovered during the macaque selections in this study had increased potency over AAV9 in the macaque CNS. Three PAL family variants, PAL1A-PAL1C, were significantly better at transducing all four lobes of the macaque brain as well as the thalamus, midbrain, and corpus callosum, but not the cerebellum, brain stem, or spinal cord (FIGS. 17E and 20A). These three variants were additionally significantly detargeted from the dorsal root ganglia (DRG) (FIG. 17E). M.Fas.1-3 did not demonstrate significantly increased potency in the cerebrum, but unlike the PAL variants, they effectively transduced the spinal cord (FIG. 17E). All macaque-derived variants except for M.Fas.3 were significantly detargeted from the macaque liver compared to AAV9 as measured by both transgene mRNA expression and vector genome delivery (FIGS. 17E and 20B).

### Individual Characterization of a PAL Variant in One Cynomolgous Macaque.

Applicant attempted to further optimize the PAL motif by performing a second-generation selection in cynomolgus macaques with the PAL motif fixed, varying only the second and sixth position of the 7-mer insert as well as the three flanking residues immediately upstream of the insert. Modifications to this upstream flanking region, corresponding to SAQ in wild-type AAV9, have previously resulted in the enhanced transduction of PHP.eB compared to PHP.B.²² From this selection Applicant chose the second-generation PAL variant PAL2, with the sequence EVGPTQGTVR (SEQ ID NO: 332), for further study due to its relatively high performance and its similarity with the top first-generation variant PAL1A. Applicant produced rAAVs with AAV9 and PAL2 each encoding hFXN under control of the CBh promoter and systemically administered 3E+13 vg/kg of each virus, for a total dose of 6E+13 vg/kg, to one female cynomolgus macaque. In order to distinguish between genomes and transcripts from the two different capsids, Applicant tagged the hFXN transgene with an HA or FLAG epitope tag in PAL2 and AAV9 capsids, respectively.

Applicant assessed both vector transgene delivery and expression throughout a variety of tissues and found that PAL2 facilitated between a fourfold and sixfold increase in transgene mRNA expression throughout the cerebrum compared to AAV9, except in the corpus callosum, where we only observed a 2.7-fold improvement (FIG. 18A and Table 8). As seen with the first-generation PAL1 variants, PAL2 transduction lagged in the cerebellum compared to the cerebrum, and in this experiment, we found only a 2.1-fold increase in mRNA expression from PAL2 in the cerebellum (FIG. 18A). Improvements in vector genome delivery were more modest; throughout the cerebrum and cerebellum we observed less than twofold more PAL2 vector genomes compared to AAV9 (FIG. 18A and Table 8). In line with our observations of first-generation PAL1 variants, PAL2 demonstrated one quarter of the vector genome abundance and one half of the mRNA expression in the liver relative to AAV9 (FIG. 18A and Table 8).

To further characterize transgene expression from PAL2, we performed immunostaining for the HA-tagged hFXN transgene. Applicant found that PAL2 transduction was broadly distributed throughout the cerebrum, and cells expressing HA-hFXN were found in diverse regions (FIG. 18B). Though AAV9 transduction in the brain is thought to be mostly limited to astrocytes rather than neurons,¹⁴⁻³³ PAL2 demonstrated distinct neurotropic behavior: HA-hFXN expression was frequently observed in NeuN+ neurons in both the cortex and hippocampus (FIG. 18C-18D). Though PAL2 also outperformed AAV9 in transgene delivery and expression in the spinal cord (FIG. 18A and Table 8), transduction in the spinal cord was more limited to non-neuronal cell types (FIG. 18E).

As rAAVs have been successfully employed in the treatment of ocular diseases,³⁴ Applicant also assessed the relative efficiency of PAL2 in the retinal pigment epithelium (RPE) and neuroretina (retina absent the RPE). Applicant found that PAL2 outperformed AAV9 in both transgene delivery and expression in the neuroretina by a factor of 3.8 and 13.4, respectively (FIG. 18A and Table 8). PAL2 vector genome abundance in the RPE was only 0.6-fold that of AAV9, but PAL2 nonetheless facilitated 2.3-fold greater mRNA expression in the RPE. Expression of the HA-hFXN transgene in the neuroretina was largely limited to photoreceptor cells, with expression particularly concentrated in the outer plexiform layer where bipolar and horizontal cells synapse with photoreceptors (FIG. 18F).

Though three first-generation PAL1 variants were significantly detargeted from the DRG, we found that PAL2 had increased DRG tropism compared to AAV9 (FIG. 18A and Table 8). Transduction of the DRG has been associated with neuroinflammation and neurodegeneration that can result in ataxia and other PNS deficits.^{17,20,35-37} Applicant therefore assessed the spinal cord and DRG for abnormal pathology. As the macaque was administered a pool containing both AAV9 and PAL2, Applicant are unable to distinguish the effects of one capsid from another; however, Applicant was able to assess the combined effect of the two vectors in the context of this experiment. Multiple DRG and spinal cord sections from the cervical, thoracic, and lumbar regions of the spine were analyzed by a neuropathologist who established severity scores ranging from 0 (within normal limits) to 5 as previously described.^{36,38} The macaque did not show abnormal pathology in any region tested (FIG. 18G).

### Discussion

In this example, Applicant used the previously described DELIVER method³⁰ to identify the novel PAL family of capsids that offer enhanced transduction in the CNS of cynomolgus macaques after a single dose IV infusion (FIGS. 17A-17E and 18A-18H). This is the first example of engineered AAVs evolved de novo in macaques demonstrating increased CNS tropism in macaques following systemic administration. Applicant identified this family of capsids after just two rounds of selection in macaques, illustrating the utility of DELIVER in identifying potent AAV capsid variants in an additional tissue type. In a pooled characterization experiment assessing the performance of multiple engineered rAAVs in macaques, three PAL capsid variants (PAL1A-C) were capable of a moderate but statistically significant two- to threefold increase in transgene expression throughout the cerebrum (FIGS. 17A-17E and 20A-20B). The second-generation variant PAL2 displayed an even greater four to six-fold improvement in transgene expression in most areas of the cerebrum in one macaque. PAL2 was notably 13-fold more potent than AAV9 at transducing the neuroretina of one macaque (FIG. 18A-18H), suggesting the feasibility of using a systemically administered rAAV to treat a disease affecting both the brain and retina, such as Krabbe disease. Additional studies with a greater number of animal subjects will be required to fully assess the performance of this variant.

In addition to demonstrating increased CNS tropism in macaques, the PAL variants displayed a striking decrease in liver tropism both in terms of vector genome delivery and transgene mRNA expression (FIGS. 17E, 20B, and 18A, and Table 8). Identification of vectors with reduced liver tropism is key to harnessing the advantages conferred by systemic administration, as sequestration of viral particles in the liver following IV infusion both decreases the effective dose at the target tissue and can lead to severe liver toxicity.^{1,2,15,17-20} These results therefore suggest that PAL vectors could achieve therapeutic efficacy following systemic administration at a reduced dose and with a lower risk of liver toxicity.

Though the PAL variants are capable of enhanced transduction of the macaque CNS, we found that engineered variants identified in mice were universally unsuccessful. Variants such as MDV1A that were selected in mice via DELIVER were able to potently transduce the CNS of two mouse strains (FIG. 15A-15H), but none of the four mouse-selected variants identified in this study outperformed AAV9 in transducing any area of the macaque CNS (FIG. 17A-17E). Even more surprisingly, AAV.CAP-B10 and AAV.CAP-B22, two variants that were selected in mice and shown to have enhanced neurotropic properties in marmosets,^{23,25} also failed to outperform AAV9 in transducing the CNS of cynomolgus macaques, a primate more closely related to humans. The failure of AAV transduction profiles to translate from mice to primates is well documented and has hampered development of CNS-targeted rAAV therapies,^{26,29} but this finding that the performance of some variants in one primate species may not translate even to another primate species has worrying implications for the field. Though variants that retain their overall transduction behavior across a variety of model organism species are powerful tools from a preclinical standpoint and have been found for the skeletal muscle,³⁰ the complexity of the CNS appears to pose additional challenges. It is therefore can be important that engineered AAVs are selected and evaluated in an appropriate animal model—one with the highest possible degree of similarity to humans—in order to maximize the likelihood of therapeutic efficacy in treating human neurological disorders.

The properties of the PAL variants and other variants identified in this study may be further enhanced in a number of ways. Firstly, additional iterations of directed evolution focusing on the 7-mer insert motif, flanking amino acids, or other areas of the capsid may result in improved or otherwise altered transduction properties as has been observed in the development of PHP.eB, AAV.CAP-B10, and AAV.CAP-B22.^{22,25} Secondly, though the advantages of systemic administration motivating this study are clear, refinement of intra-CSF delivery routes remains a promising area of research and may result in more robust transgene expression in the CNS³³ at the possible expense of a higher risk of neuroinflammation and neurodegeneration.^{35-37,39} The combination of a PAL variant with an intra-CSF delivery method such as intrathecal or intracisternal injection may prove fruitful and suggest more varied applications for these variants. Finally, the inclusion of tissue-specific microRNA targets on the vector transgene can reduce transgene expression and associated side effects in off-target tissues. Similar strategies utilizing microRNAs have shown promising results in vivo in the context of both liver and DRG detargeting.^{38,40-43}

In summary, this Example identifies of a variety of AAV capsid variants with neurotropic properties in either mice or cynomolgus macaques, including a more extensively characterized family of variants containing a PAL motif that are capable of enhanced transduction of the macaque CNS and reduced sequestration in the liver following a single IV infusion. These results suggest that rAAV-based therapies with PAL variants may achieve therapeutic efficacy at a reduced dose, minimizing both safety concerns and vector manufacturing challenges. Applicant additionally provides a list of the 1000 most highly enriched capsid variants in the CNS of macaques and two mouse strains (Table 8); further investigation and characterization of these variants may identify additional candidates for CNS gene therapy. Though Applicant was unable to identify any variants able to potently transduce both the mouse and macaque CNS, this finding indicates a critical need for appropriate animal models and a move away from the current paradigm of evolving CNS-tropic AAVs in mice. This Example, particularly the characterization of the PAL family of variants in macaques, represents a significant advancement towards safe and effective rAAV therapies for diseases of the CNS in humans.

### Methods

### Animals

All animal care, housing, and experimental procedures were carried out in accordance with the Broad Institute Institutional Animal Care and Use Committee (IACUC) and Biomere's IACUC.

### Mice

Eight week old male and female C57BL/6J (JAX, #000664) and BALB/cJ (JAX, #000651) mice were purchased from the Jackson laboratory. All mouse AAV injections were performed retro-orbitally. Tissue samples were collected from the mice two weeks post-injection after whole body perfusion with either Dulbecco's phosphate-buffered saline (DPBS) (Gibco, #14190144) or DPBS followed by 4% paraformaldehyde (PFA).

### Cynomolgus Macaques

Non-human primate studies were performed at Biomere (Worcester, MA, USA) in accordance with their standard operating protocols and procedures approved by their IACUC. Male and female cynomolgus macaques, approximately 2 years of age, with a serum AAV9 neutralizing antibody titer of less than 1:3 were selected for in vivo studies. For all experiments, macaques were injected via an IV bolus injection. Animals were euthanized after 3 weeks and perfused with DPBS, after which CNS, muscle, and organ tissues were harvested. Tissue samples were preserved in RNAlater stabilization solution (Invitrogen, #AM7024) prior to downstream processing.

### Constructs

CMV-EGFP plasmids used to produce EGFP-encoding AAV9 and MDV1A were generated by cloning the cytomegalovirus (CMV) promoter, EGFP coding sequence, and bovine growth hormone polyadenylation signal (bGH pA) into the pZac2.1 construct purchased from the University of Pennsylvania vector core. The AAV capsid library recipient plasmid was generated by assembling the human synapsin 1 (hSyn) promoter, AAV2 rep, AAV9 cap, and SV40 polyadenylation signal into an ITR-containing backbone. The AAV9 cap gene on the library recipient plasmid was modified to contain BsmBI restriction sites immediately after Q486 and Q588 to facilitate insertion of a variable peptide sequence. The pZac2.1-CBh-hFXN-HA-bGH and pZac2.1-CBh-hFXN-FLAG-bGH plasmids were assembled by cloning the hybrid CBh promoter,⁴⁴ human frataxin coding sequence, HA tag, and bGH pA into the pZac2.1 plasmid backbone between the ITRs. As previously described, for the pooled characterization experiment, 12 bp barcodes were inserted immediately after the HA tag in the pZac2.1-CBh-hFXN-HA-bGH plasmid.³⁰ Each variant in the pooled characterization experiment was associated with 50 unique barcodes that were randomly generated with a minimum Hamming distance of four between any two barcodes.

First round AAV capsid library plasmids were prepared by amplifying a section of the AAV9 cap gene with an NNK degenerate reverse primer to produce fragments encoding every possible random 7-mer peptide insertion after Q588. These fragments were then introduced into the BsmBI-digested capsid library recipient plasmid. This library has a theoretical diversity of 20⁷ (1.28E+9) variants at the amino acid level, and we were able to identify at least 5E+6 unique capsid variants in our first-round capsid libraries based on next-generation sequencing. Second round libraries were generated through a similar method, but instead of NNK degenerate primers, a synthetic oligo pool (Agilent, Santa Clara, CA) was used to produce only selected variants of interest and synonymous DNA codon replicates. Libraries with the fixed PAL motif X₁X₂X₃PX₄QGTX₅R were generated with a reverse primer containing NNK degenerate codons at the variable positions X₁-X₅. All cloning was performed using the NEBuilder HiFi DNA assembly master mix (New England Biolabs, Ipswitch, MA).

### Capsid Library and Recombinant AAV Production

AAV capsid libraries and rAAVs were produced in HEK293 cells (CRL-1573, ATCC, Mannassas, VA) with the usual triple-plasmid transfection method.⁴⁵ Briefly, HEK293 cells were seeded into 15 cm dishes at a density of 2E+7 cells per dish and transfected the following day using PEI MAX (Polysciences, Warrington, PA). For individual rAAV production, cells were transfected with 16 μg pALDX-80 (Aldevron, Fargo, ND), 8 μg Rep2/Cap plasmid, and 8 μg of the ITR-containing transgene plasmid per dish. rAAVs were harvested from the cells and media and purified by ultracentrifugation over an iodixanol gradient as previously described.⁴⁵ A slightly modified protocol was used for the production of AAV capsid libraries. First, only 10 ng of the AAV capsid plasmid library was used per dish in order to prevent cross-packaging of variants and the formation of mosaic capsids, and 8 μg of pUC19 plasmid was included in the transfection to maintain the total amount of transfected plasmid. Second, 8 μg of Rep-AAP plasmid (a generous gift from Benjamin Deverman)²¹ was used in place of the Rep2/Cap plasmid. Finally, virus was harvested after 60 hours rather than the usual 120 hours in order to limit secondary transduction of virus-producing cells. All AAVs were titered by qPCR.

### In Vivo Selection in Mice and Cynomolgus Macaques

First- and second-round selections were performed in eight week old C57BL/6J and BALB/cJ mice and in two year old macaques. Six male and six female mice from each strain were used for each selection, and each mouse received a 1E+12 vg injected dose of either the AQ or DG capsid variant library. For the first round of selection in macaques, one male and one female were injected with 1E+13 vg/kg capsid library. For the second round of selection in macaques, two males and one female were injected with 3E+13 vg/kg AQ capsid library. For selection on the fixed PAL motif with modified flanking amino acids in macaques, two males were injected with 3E+13 vg/kg. In all selection experiments, three weeks after injection, animals were euthanized by perfusion with saline and whole brains were harvested. Spinal cords were additionally harvested from macaques. Fresh tissues were cut into 2 mm cubes and snap-frozen in liquid nitrogen before being stored at −80° C. Total RNA was extracted from at least 80% of the total tissue volume with TRIzol (Thermo Fisher, Waltham, MA) and mRNA was enriched from total RNA samples with oligo dT beads (New England Biolabs) and treated with Turbo DNase (Thermo Fisher). Subsequently, cDNA was synthesized with SuperScript IV reverse transcriptase (Thermo Fisher) and a capsid-specific primer (5′-GAAAGTTGCCGTCCGTGTGAGG-3′ (SEQ ID NO: 8590)). Capsid variant sequences were then amplified with Q5 High-Fidelity 2× master mix (New England Biolabs) and primers flanking the 7-mer insert (5′-ACAAGTGGCCACAAACCACCA-3′ (SEQ ID NO: 8591) and 5′-GGTTTTUAACCCAGCCGGTC-3′ (SEQ ID NO: 8592)) that added Illumina adaptors and unique indices (New England Biolabs). Amplicons were pooled at an equimolar ratio and sequenced on an Illumina NextSeq.

In Vivo rAAV Characterization

For comparison of vector genome delivery and transgene mRNA expression between AAV9 and MDV1A in mice, four male and four female 8 week old C57BL/6J mice and four male and four female 8 week old BALB/cJ mice were injected with 1E+12 vg of AAV9- or MDV1A-CMV-EGFP. Tissues were harvested two weeks after injection. For comparison of transgene expression via immunostaining, 8 week old C57BL/6J and BALB/cJ mice were injected with 5E+11 vg of AAV9- or MDV1A-CMV-EGFP. Tissues were again harvested two weeks after injection. For comparison of PAL2 and AAV9, one male two year old macaque was injected with 3E+13 vg/kg each of AAV9-CBh-hFXN-FLAG and PAL2-CBh-hFXN-HA. The macaque was euthanized by saline perfusion and tissues were harvested 3 weeks after injection.

For the pooled rAAV characterization experiment, eight in-house macaque-derived capsids, four in-house mouse-derived capsids, AAV.CAP-B10, AAV.CAP-B22, PHP.C2, and AAV9 were used to produce rAAVs packaging the barcoded CBh-hFXN-HA-bGH transgene. Equal amounts of each of the 16 barcoded rAAV pools were mixed and injected into two male and one female two year old macaques, three male and four female 8 week old C57BL/6J mice, and two male and two female 8 week old BALB/cJ mice. All animals were injected with a combined dose of 3E+13 vg/kg, or 1.875E+12 vg/kg per capsid variant. Animals were euthanized by saline perfusion and tissues were harvested 4 weeks after injection and total RNA was extracted and treated as described above, and macaque liver DNA was additionally isolated with QuickExtract DNA extract solution (Lucigen, Middleton, WI). cDNA was synthesized with a bGH pA-specific primer (5′-TTCACTGCATTCTAGTTGTGGTTTG-3′ (SEQ ID NO: 8583)) and DNA and cDNA were amplified with Q5 High-Fidelity 2X master mix and primers flanking the barcode region (5′-CCATACGATGTTCCAGATTACGC-3′ (SEQ ID NO: 8594) and 5′-CAATGTATCTTATCATGTCTGCTCGA-3′ (SEQ ID NO: 8595)). Amplicons with Illumina adapters and unique indices were pooled at equimolar ratios and sequenced on an Illumina NextSeq.

### Next Generation Sequencing Data Analysis

Next generation sequencing analysis of the results of selection experiments was performed as previously described.³⁰ Briefly, Illumina sequencing reads were demultiplexed with bcl2fastq2-v2.17.1 and the 21 bp variant sequence was extracted from each read. Variants were counted in each sample and normalized to the sequencing depth of the run to assign each variant a reads per million (RPM) score. Variants were ranked according to the ratio of variant RPM in the sample to variant RPM in the matched sequenced virus library sample to account for unequal distribution of variants in the injected virus library. The highest scoring amino acid variants from the first round of selection in each animal model (10,000 from mice and 20,000 from cynomolgus macaques) were chosen for the second round selection. For each such amino acid variant, a sequence encoding the same peptide by synonymous DNA codons was included in the design of the second-round library to control for DNA sequence-specific effects. For variants with multiple synonymous sequences already observed in experimental samples, the highest scoring synonymous variant was included. For other variants, an artificial sequence was generated by randomizing each codon in the original sequence to a synonymous codon where possible. 5% of variants in the second round library encoded stop codons and were artificially added to the library to control for cross-packaging events during virus library production. Following the second round of selection, DNA sequence variants were ranked as described above, and amino acid variants were ranked according to the sum of the ranks of the two corresponding synonymous sequences. Variants identified in the selection with the fixed PAL motif were ranked as in the first round of selection.

For the pooled rAAV characterization experiment, a ratio was calculated for each barcode of the sample RPM to the RPM of that barcode in the matched sequenced virus library. For each capsid variant, the 10 strongest and 5 weakest barcodes across all samples in the sequencing run were identified according to this metric and removed as outliers from downstream analysis. The remaining 35 midrange barcodes for each variant were then used to determine average transgene expression in each sample as described above.

### Macaque Variant Clustering

Pairwise dissimilarity scores between the top 1000 CNS-tropic capsid variants (corrected for synonymous DNA codon sequences) were calculated by adding the single-residue substitution score at each of the seven positions according to the BLOSUM62 substitution matrix. A matrix of dissimilarity scores was converted into a distance matrix by computing the distance metric d(s, t) between any two peptide sequences s and t by analogy with the scalar product as follows:

$$d ( s , t ) = \sqrt{〈 s \vert s 〉 + 〈 t \vert t 〉 - 2 〈 s \vert t 〉}$$

where [symbol] s|t [symbol] is the dissimilarity score between s and t.⁴⁶ This distance matrix was then used for k-medoids clustering with the scikit-learn package. The number of clusters k was chosen by maximizing the silhouette score.

### Computational Protein Modeling

Computational modeling of the VR-VIII loop of MDV1A, MDV1B, PAL1A, and PAL-like.1 was performed on the ProMod3-powered SWISS-MODEL server.^{47,48} AAV9 was used as a template for homology modeling (PDB: 3UX1)³² and all structures were visualized in PyMOL.

### Transgene Delivery and Expression Quantification

For transgene expression quantification, RNA was extracted from mouse and macaque tissues with TRIzol (Thermo Fisher) and treated with Turbo DNase (Thermo Fisher). cDNA was synthesized with SuperScript IV reverse transcriptase (Thermo Fisher) with an oligo-dT primer. For transgene delivery (vector genome quantification) experiments, DNA was extracted from mouse and macaque tissues with QuickExtract DNA extract solution (Lucigen) following pulverization of snap-frozen tissue with a Geno/Grinder 2010 (SPEX SamplePrep, Metuchen, NJ). Transgene mRNA and DNA were measured by qPCR using Taqman assays specific to the transgene (EGFP or HA- or FLAG-tagged hFN) mRNA or DNA or a housekeeping control (GAPDH). All measurements were quantified based on a standard curve generated by amplifying a gblock containing the target sequence of each Taqman assay, and absolute quantities of transgene mRNA and DNA were then normalized to the housekeeping gene.

### Histology

### Tissue Preparation

Whole brains harvested from mice were fixed in 4% PFA for 1 h at room temperature, washed with DPBS, and cryoprotected in 30% sucrose at 4° C. overnight. Tissues harvested from the macaque injected with PAL2- and AAV9-CBh-hFXN were fixed in 4% PFA overnight at 4° C. and washed 3 times with DPBS. Fixed macaque tissues were cryoprotected in 15% sucrose at 4° C. overnight and then 30% sucrose at 4° C. for up to 3 days. Cryoprotected tissues were then embedded in O.C.T. compound (Sakura Finetek USA, Torrance, CA) and snap frozen in liquid nitrogen-chilled isopentane. Frozen tissue blocks were sectioned at a thickness of 12 μm on a CM1860 cryostat (Leica Biosystems, Wetzlar, Germany) and mounted onto Superfrost Plus slides (VWR, Radnor, PA). Whole 5 mm coronal slabs of fixed macaque brain hemispheres were embedded in 4% low melting point agarose (Sigma-Aldrich, St. Louis, MO) and 40 μm free-floating sections were collected in DPBS using a VT1000S vibrating blade microtome (Leica Biosystems).

### Immunohistochemistry

IHCs were performed with an HRP micropolymer kit (ab236466, Abcam, Cambridge, UK) according to the manufacturer's instructions except where noted below. All primary antibody incubations on cryosections were performed at 4° C. overnight in blocking buffer containing 5% normal goat serum, 2% bovine serum albumin, 2% M.O.M. protein concentrate (Vector Labs, Burlingame, CA), and 0.1% Tween-20. Mouse brain cryosections were stained with a 1:1000 diluted rabbit anti-GFP primary antibody (A11122, Thermo Fisher). Following primary antibody incubation, sections were washed three times with PBS and incubated with HRP conjugate at RT for 30 minutes.

To visualize cells in the macaque brain expressing HA- or FLAG-tagged hFXN transgene, IHC was performed on 40 μm free-floating sections. Sections were blocked at room temperature for 1 hour in blocking buffer containing 5% normal goat serum with 0.2% Triton X-100 and then incubated with 1:7500 rabbit anti-HA antibody (ab9110, Abcam) in the same blocking buffer overnight at 4° C. with agitation. Following primary antibody incubation, sections were washed and incubated with 25% v/v HRP conjugate (ab236466, Abcam) diluted in PBS overnight at 4° C. with agitation.

For all IHC experiments, sections were washed with PBS following incubation with HRP conjugate and the signal was visualized with 3,3′-diaminobenzidine (Abcam) prepared according to the manufacturer's instructions for 3 minutes at RT. The free-floating macaque brain sections were then mounted onto HISTOBOND+ slides (Marienfeld, Lauda-Königshofen, Germany). Mouse sections were mounted with VectaMount AQ (Vector Labs). Macaque sections were dehydrated in a graded ethanol series, cleared with three changes of CitriSolv (Decon Laboratories, King of Prussia, PA), and mounted with VectaMount (Vector Labs). Sections were imaged on an EVOS M7000 all-in-one microscope using a 4× objective lens.

### Immunofluorescence

Cryosections of macaque brain, spinal cord, and neuroretina tissue were permeabilized for 10 minutes in 5% normal goat serum with 0.2% Triton X-100 before being blocked at room temperature for 1 hour in blocking buffer containing 5% normal goat serum, 2% bovine serum albumin, 2% M.O.M. protein concentrate (Vector Labs), and 0.1% Tween-20. Primary antibody incubations were performed overnight at 4° C. in blocking buffer; retina sections were labeled with 1:500 rabbit anti-HA (MA5-27915, Thermo Fisher) and 1:1000 mouse anti-rhodopsin (MA1-722, Thermo Fisher) antibodies, brain and spinal cord sections were labeled with 1:250 rabbit anti-HA (MA5-27915, Thermo Fisher) and 1:1000 mouse anti-NeuN (MA5-33103, Thermo Fisher) antibodies. Sections were washed three times with PBS before being incubated at room temperature for 30 minutes in blocking buffer with 1:500 goat anti-rabbit Alexa Fluor 488 (A11034, Thermo Fisher) and 1:500 goat anti-mouse Alexa Fluor 594 (A32742, Thermo Fisher) secondary antibodies. Brain and spinal cord sections were mounted with VECTASHIELD antifade mounting media (Vector Labs). Retina sections were treated with the TrueVIEW autofluorescence quenching kit (Vector Labs) according to the manufacturer's instructions and mounted with VECTASHIELD Vibrance antifade mounting media (Vector Labs). Sections were imaged on an EVOS M7000 all-in-one microscope using a 20X objective lens. Linear contrast adjustments were applied to images.

### Pathology

Macaque spinal cord and DRG sections were stained with hematoxylin and eosin (ab245880, Abcam) according to the usual method. A board-certified neuropathologist who was blinded to the experimental design reviewed anonymized slides and assigned a severity score between 0 (within normal limits) and five as previously described.³⁸ Severity scores were established for the spinal cord and DRG on sections from three segments each from the cervical, thoracic, and lumbar regions.

### Statistical Analysis

All statistical analyses were performed in GraphPad Prism v8 (GraphPad Software, San Diego, CA). All data are presented as mean±SD where applicable. Datasets were tested for normality using the Shapiro-Wilk test at a significance level of 0.01. All datasets were tested for outliers using the ROUT method and Q=0.5%. Outliers were identified and removed from AAV9-injected female C57BL6J spinal cord RNA and DNA (one outlier each, FIG. 15A-15F) and C57BL/6J brain and liver RNA (one outlier each where the entire animal was removed from analysis, FIG. 17A-17E). For comparisons between AAV9 and MDV1A (FIG. 15D-15E), differences were tested for significance with Welch's t-test with Holm-Šidák correction for multiple comparisons. For comparisons between AAV9 and multiple other variants (FIGS. 17A-17E and 20A-20B), differences were tested for significance with a one-way ANOVA assuming equal variance and Dunnett's multiple comparison test using AAV9 as a control mean. All statistical tests were performed on raw data without normalization to AAV9, though mRNA expression data are presented normalized to the mean of AAV9 expression for greater interpretability.

### References Related for Example 10

1. Deverman, B. E., Ravina, B. M., Bankiewicz, K. S., Paul, S. M., and Sah, D. W. Y. (2018). Gene therapy for neurological disorders: progress and prospects. Nat Rev Drug Discov 17, 641-659.
2. Ojala, D. S., Amara, D. P., and Schaffer, D. V. (2015). Adeno-Associated Virus Vectors and Neurological Gene Therapy. Neuroscientist 21, 84-98.
3. Wu, Z., Asokan, A., and Samulski, R. J. (2006). Adeno-associated Virus Serotypes: Vector Toolkit for Human Gene Therapy. Molecular Therapy 14, 316-327.
4. Naso, M. F., Tomkowicz, B., Perry, W. L., and Strohl, W. R. (2017). Adeno-Associated Virus (AAV) as a Vector for Gene Therapy. BioDrugs 31, 317-334.
5. Hocquemiller, M., Giersch, L., Audrain, M., Parker, S., and Cartier, N. (2016). Adeno-Associated Virus-Based Gene Therapy for CNS Diseases. Hum Gene Ther 27, 478-496.
6. Afione, S. A., Conrad, C. K., Kearns, W. G., Chunduru, S., Adams, R., Reynolds, T. C., Guggino, W. B., Cutting, G. R., Carter, B. J., and Flotte, T. R. (1996). In vivo model of adeno-associated virus vector persistence and rescue. J Virol 70, 3235-3241.
7. Mendell, J. R., Al-Zaidy, S., Shell, R., Arnold, W. D., Rodino-Klapac, L. R., Prior, T. W., Lowes, L., Alfano, L., Berry, K., Church, K., et al. (2017). Single-Dose Gene-Replacement Therapy for Spinal Muscular Atrophy. N Engl J Med 377, 1713-1722.
8. Foust, K. D., Wang, X., McGovern, V. L., Braun, L., Bevan, A. K., Haidet, A. M., Le, T. T., Morales, P. R., Rich, M. M., Burghes, A. H. M., et al. (2010). Rescue of the spinal muscular atrophy phenotype in a mouse model by early postnatal delivery of SMN. Nat Biotechnol 28, 271-274.
9. Leone, P., Shera, D., McPhee, S. W. J., Francis, J. S., Kolodny, E. H., Bilaniuk, L. T., Wang, D.-J., Assadi, M., Goldfarb, O., Goldman, H. W., et al. (2012). Long-Term Follow-Up After Gene Therapy for Canavan Disease. Science Translational Medicine.
10. Hwu, W.-L., Muramatsu, S., Tseng, S.-H., Tzen, K.-Y., Lee, N.-C., Chien, Y.-H., Snyder, R. O., Byrne, B. J., Tai, C.-H., and Wu, R.-M. (2012). Gene Therapy for Aromatic 1-Amino Acid Decarboxylase Deficiency. Science Translational Medicine.
11. Saraiva, J., Nobre, R. J., and Pereira de Almeida, L. (2016). Gene therapy for the CNS using AAVs: The impact of systemic delivery by AAV9. Journal of Controlled Release 241, 94-109.
12. Zincarelli, C., Soltys, S., Rengo, G., and Rabinowitz, J. E. (2008). Analysis of AAV Serotypes 1-9 Mediated Gene Expression and Tropism in Mice After Systemic Injection. Molecular Therapy 16, 1073-1080.
13. Gray, S. J., Woodard, K. T., and Samulski, R. J. (2010). Viral vectors and delivery strategies for CNS gene therapy. Ther Deliv 1, 517-534.
14. Foust, K. D., Nurre, E., Montgomery, C. L., Hernandez, A., Chan, C. M., and Kaspar, B. K. (2009). Intravascular AAV9 preferentially targets neonatal-neurons and adult-astrocytes in CNS. Nat Biotechnol 27, 59-65.
15. Huang, L., Wan, J., Wu, Y., Tian, Y., Yao, Y., Yao, S., Ji, X., Wang, S., Su, Z., and Xu, H. (2021). Challenges in adeno-associated virus-based treatment of central nervous system diseases through systemic injection. Life Sciences 270, 119142.
16. Liu, D., Zhu, M., Zhang, Y., and Diao, Y. (2021). Crossing the blood-brain barrier with AAV vectors. Metab Brain Dis 36, 45-52.
17. Hinderer, C., Katz, N., Buza, E. L., Dyer, C., Goode, T., Bell, P., Richman, L. K., and Wilson, J. M. (2018). Severe Toxicity in Nonhuman Primates and Piglets Following High-Dose Intravenous Administration of an Adeno-Associated Virus Vector Expressing Human SMN. Hum Gene Ther 29, 285-298.
18. Gao, G., Lu, Y., Calcedo, R., Grant, R. L., Bell, P., Wang, L., Figueredo, J., Lock, M., and Wilson, J. M. (2006). Biology of AAV Serotype Vectors in Liver-Directed Gene Transfer to Nonhuman Primates. Molecular Therapy 13, 77-87.
19. Morales, L., Gambhir, Y., Bennett, J., and Stedman, H. H. (2020). Broader Implications of Progressive Liver Dysfunction and Lethal Sepsis in Two Boys following Systemic High-Dose AAV. Molecular Therapy 28, 1753-1755.
20. Palazzi, X., Pardo, I., Sirivelu, M., Newman, L., Kumpf, S., Qian, J., Franks, T., Lopes, S., Liu, J., Monarski, L., et al. (2021). Biodistribution and Tolerability of AAV-PHP.B-CBh-SMN1 in Wistar Han Rats and Cynomolgus Macaques Reveal Different Toxicologic Profiles. Hum Gene Ther.
21. Deverman, B. E., Pravdo, P. L., Simpson, B. P., Kumar, S. R., Chan, K. Y., Banerjee, A., Wu, W.-L., Yang, B., Huber, N., Pasca, S. P., et al. (2016). Cre-dependent selection yields AAV variants for widespread gene transfer to the adult brain. Nat Biotechnol 34, 204-209.
22. Chan, K. Y., Jang, M. J., Yoo, B. B., Greenbaum, A., Ravi, N., Wu, W.-L., Sanchez-Guardado, L., Lois, C., Mazmanian, S. K., Deverman, B. E., et al. (2017). Engineered AAVs for efficient noninvasive gene delivery to the central and peripheral nervous systems. Nat. Neurosci. 20, 1172-1179.
23. Kumar, S. R., Miles, T. F., Chen, X., Brown, D., Dobreva, T., Huang, Q., Ding, X., Luo, Y., Einarsson, P. H., Greenbaum, A., et al. (2020). Multiplexed Cre-dependent selection yields systemic AAVs for targeting distinct brain cell types. Nat Methods 17, 541-550.
24. Nonnenmacher, M., Wang, W., Child, M. A., Ren, X.-Q., Huang, C., Ren, A. Z., Tocci, J., Chen, Q., Bittner, K., Tyson, K., et al. (2021). Rapid evolution of blood-brain-barrier-penetrating AAV capsids by RNA-driven biopanning. Molecular Therapy—Methods & Clinical Development 20, 366-378.
25. Goertsen, D., Flytzanis, N. C., Goeden, N., Chuapoco, M. R., Cummins, A., Chen, Y., Fan, Y., Zhang, Q., Sharma, J., Duan, Y., et al. (2022). AAV capsid variants with brain-wide transgene expression and decreased liver targeting after intravenous delivery in mouse and marmoset. Nat Neurosci 25, 106-115.
26. Hordeaux, J., Wang, Q., Katz, N., Buza, E. L., Bell, P., and Wilson, J. M. (2018). The Neurotropic Properties of AAV-PHP.B Are Limited to C57BIJ6J Mice. Molecular Therapy 26, 664-668.
27. Huang, Q., Chan, K. Y., Tobey, I. G., Chan, Y. A., Poterba, T., Boutros, C. L., Balazs, A. B., Daneman, R., Bloom, J. M., Seed, C., et al. (2019). Delivering genes across the blood-brain barrier: LY6A, a novel cellular receptor for AAV-PHP.B capsids. PLOS ONE 14, e0225206.
28. Mathiesen, S. N., Lock, J. L., Schoderboeck, L., Abraham, W. C., and Hughes, S. M. (2020). CNS Transduction Benefits of AAV-PHP.eB over AAV9 Are Dependent on Administration Route and Mouse Strain. Molecular Therapy—Methods & Clinical Development 19, 447-458.
29. Matsuzaki, Y., Konno, A., Mochizuki, R., Shinohara, Y., Nitta, K., Okada, Y., and Hirai, H. (2018). Intravenous administration of the adeno-associated virus-PHP.B capsid fails to upregulate transduction efficiency in the marmoset brain. Neuroscience Letters 665, 182-188.
30. Tabebordbar, M., Lagerborg, K. A., Stanton, A., King, E. M., Ye, S., Tellez, L., Krunnfusz, A., Tavakoli, S., Widrick, J. J., Messemer, K. A., et al. (2021). Directed evolution of a family of AAV capsid variants enabling potent muscle-directed gene delivery across species. Cell 184, 4919-4938.e22.
31. B6mer, K., Kienle, E., Huang, L.-Y., Weinmann, J., Sacher, A., Bayer, P., Stillein, C., Fakhiri, J., Zimmermann, L., Westhaus, A., et al. (2020). Pre-arrayed Pan-AAV Peptide Display Libraries for Rapid Single-Round Screening. Molecular Therapy 28, 1016-1032.
32. DiMattia, M. A., Nam, H.-J., Vliet, K. V., Mitchell, M., Bennett, A., Gurda, B. L., McKenna, R., Olson, N. H., Sinkovits, R. S., Potter, M., et al. (2012). Structural Insight into the Unique Properties of Adeno-Associated Virus Serotype 9. Journal of Virology.
33. Samaranch, L., Salegio, E. A., San Sebastian, W., Kells, A. P., Foust, K. D., Bringas, J. R., Lamarre, C., Forsayeth, J., Kaspar, B. K., and Bankiewicz, K. S. (2012). Adeno-Associated Virus Serotype 9 Transduction in the Central Nervous System of Nonhuman Primates. Human Gene Therapy 23, 382-389.
34. Aguirre, G. D. (2017). Concepts and Strategies in Retinal Gene Therapy. Investigative Ophthalmology & Visual Science 58, 5399-5411.
35. Hordeaux, J., Hinderer, C., Goode, T., Buza, E. L., Bell, P., Calcedo, R., Richman, L. K., and Wilson, J. M. (2018). Toxicology Study of Intra-Cistema Magna Adeno-Associated Virus 9 Expressing Iduronate-2-Sulfatase in Rhesus Macaques. Mol Ther Methods Clin Dev 10, 68-78.
36. Hordeaux, J., Buza, E. L., Dyer, C., Goode, T., Mitchell, T. W., Richman, L., Denton, N., Hinderer, C., Katz, N., Schmid, R., et al. (2020). Adeno-Associated Virus-Induced Dorsal Root Ganglion Pathology. Human Gene Therapy 31, 808-818.
37. Perez, B. A., Shutterly, A., Chan, Y. K., Byrne, B. J., and Corti, M. (2020). Management of Neuroinflammatory Responses to AAV-Mediated Gene Therapies for Neurodegenerative Diseases. Brain Sciences 10, 119.
38. Hordeaux, J., Buza, E. L., Jeffrey, B., Song, C., Jahan, T., Yuan, Y., Zhu, Y., Bell, P., Li, M., Chichester, J. A., et al. (2020). MicroRNA-mediated inhibition of transgene expression reduces dorsal root ganglion toxicity by AAV vectors in primates. Science Translational Medicine 12, eaba9188.
39. Novartis announces AVXS-101 intrathecal study update Novartis. https://www.novartis.com/news/media-releases/novartis-announces-avxs-101-intrathecal-study-update.
40. Geisler, A., Jungmann, A., Kurreck, J., Poller, W., Katus, H. A., Vetter, R., Fechner, H., and Müller, O. J. (2011). microRNA122-regulated transgene expression increases specificity of cardiac gene transfer upon intravenous delivery of AAV9 vectors. Gene Ther 18, 199-209.
41. Qiao, C., Yuan, Z., Li, J., He, B., Zheng, H., Mayer, C., Li, J., and Xiao, X. (2011). Liver-specific microRNA-122 target sequences incorporated in AAV vectors efficiently inhibits transgene expression in the liver. Gene Ther 18, 403-410.
42. Xie, J., Xie, Q., Zhang, H., Ameres, S. L., Hung, J.-H., Su, Q., He, R., Mu, X., Seher Ahmed, S., Park, S., et al. (2011). MicroRNA-regulated, Systemically Delivered rAAV9: A Step Closer to CNS-restricted Transgene Expression. Molecular Therapy 19, 526-535.
43. Geisler, A., and Fechner, H. (2016). MicroRNA-regulated viral vectors for gene therapy. World J Exp Med 6, 37-54.
44. Gray, S. J., Foti, S. B., Schwartz, J. W., Bachaboina, L., Taylor-Blake, B., Coleman, J., Ehlers, M. D., Zylka, M. J., McCown, T. J., and Samulski, R. J. (2011). Optimizing Promoters for Recombinant Adeno-Associated Virus-Mediated Gene Expression in the Peripheral and Central Nervous System Using Self-Complementary Vectors. Hum Gene Ther 22, 1143-1153.
45. Challis, R. C., Ravindra Kumar, S., Chan, K. Y., Challis, C., Beadle, K., Jang, M. J., Kim, H. M., Rajendran, P. S., Tompkins, J. D., Shivkumar, K., et al. (2019). Systemic AAV vectors for widespread and targeted gene delivery in rodents. Nat Protoc 14, 379-414.
46. Fischer, I. (2002). Similarity-preserving Metrics for Amino-acid Sequences. In.
47. Studer, G., Tauriello, G., Bienert, S., Biasini, M., Johner, N., and Schwede, T. (2021). ProMod3—A versatile homology modelling toolbox. PLOS Computational Biology 17, e1008667.
48. Waterhouse, A., Bertoni, M., Bienert, S., Studer, G., Tauriello, G., Gumienny, R.,
Heer, F. T., de Beer, T. A. P., Rempfer, C., Bordoli, L., et al. (2018). SWISS-MODEL: homology modelling of protein structures and complexes. Nucleic Acids Research 46, W296-W303.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A composition comprising:
a targeting moiety effective to target a central nervous system (CNS) cell, wherein the targeting moiety comprises an n-mer insert optionally comprising or consisting of a P-motif or a double valine motif, or both,
wherein the P-motif comprises or consists of the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7,
wherein the double valine motif comprises or consists of the amino acid sequence XₘX₁X₂VX₃X₄VX₅Xₙ wherein X₁, X₂, X₃, Xₘ, and Xₙ, are each independently selected from any amino acid, wherein m is 0, 1, 2, or 3, and wherein n is 0, 1, 2, 3, 4, 5, 6, or 7; and
optionally a cargo, wherein the cargo is coupled to or is otherwise associated with the targeting moiety.

2. The composition of claim 1, wherein X₂ of the P motif is Q, P, E, or H.

3. The composition of claim 1, wherein X₁ of the P motif is a polar amino acid, optionally a polar uncharged amino acid.

4. The composition of claim 1, wherein X₃ of the P motif is a nonpolar amino acid.

5. The composition of claim 1, wherein X₁ of the double valine motif is R, K, V, or W.

6. The composition of claim 1, wherein X₂ of the double valine motif is T, S, V, Y or R.

7. The composition of claim 1, wherein X₃ of the double valine motif is G, P, or S.

8. The composition of claim 1, wherein X₄ of the double valine motif is S, D, or T.

9. The composition of claim 1, wherein X₅ of the double valine motif is Y, G, S, or L.

10. The composition of claim 1, wherein the targeting moiety comprises two or more n-mer inserts, optionally wherein each n-mer insert comprises or consists of a P-motif, wherein at least one of the P-motifs comprise or consists of the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7, optionally wherein X₂ of the P motif is Q, P, E, or H, optionally wherein the X₁ of the P motif is a polar amino acid, optionally a polar uncharged amino acid, and optionally wherein X₃ of the P motif is a nonpolar amino acid.

11. The composition of claim 1, wherein the n-mer insert(s) and/or at least one P-motif and/or double valine motif is selected from any one n-mer insert and/or is encoded by a polynucleotide as set forth in one or more of SEQ ID NOs: 332-582 (Table 7), SEQ ID NOs: 583-8578 (Table 8), SEQ ID NOs: 3-819, 21-22, 24, 200, 202, 204, 212, 218, 224, 226, 228, 286, 234, 258, 260, 647, 649, 923, 1069, 1077, 1265, 2439, 2529, 2759, 3283, 3553, 3923, 4005, 4173, 4537, 4593, 4599, 4601, 4605, 4619, 4665, 4751, 4759, 4825, 4909, 4933, 5013, 5091, 5107, 5127, 5131, 5165, 5177, 5181, 5187, 5189, 5191, 5277, 5287, 5401, 5433, 5631, 5633, 5731, 5741, 5937, 6019, 6045, 6139, 6169, 6497, 7335, 8033, 8269, 8596-8613, (FIGS. 15A, 15B, 17A, 16A, 16B, 16C, and 19A-19C).

12. The composition of claim 1, wherein the n-mer insert is 3-25 or 3-15 amino acids in length.

13. The composition of claim 1, wherein
a. X₁ of the P motif is S, T, N, Q, C, Y or A,
b. X₂ of the P motif is Q, P, E, or H,
c. X₃ is G, A, M, W, L, V, F, or I, or
d. any combination thereof.

14. The composition of claim 1, wherein the targeting moiety comprises a polypeptide, a polynucleotide, a lipid, a polymer, a sugar, or any combination thereof, wherein the polypeptide, the polynucleotide, the lipid, the polymer, the sugar, or any combination thereof is operably coupled to the n-mer insert(s).

15. The composition of claim 1, wherein the targeting moiety comprises a viral protein.

16. The composition of claim 15, wherein the viral protein is a capsid protein.

17. The composition of claim 15, wherein the n-mer insert(s) is/are incorporated into the viral protein such that at least the n-mer insert is located between two amino acids of the viral protein such that at least the n-mer insert is external to a viral capsid.

18. The composition of claim 15, wherein the viral protein is an adeno associated virus (AAV) protein.

19. The composition of claim 18, wherein the AAV protein is an AAV capsid protein.

20. The composition of claim 19, wherein one or more of the n-mer insert(s) are each incorporated into the AAV protein such that the n-mer insert, optionally the P motif(s) and/or double valine motif(s), is/are inserted between any two contiguous amino acids independently selected from amino acids 262-269, 327-332, 382-386, 452-460, 488-505, 527-539, 545-558, 581-593, 598-599, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

21. The composition of claim 19, wherein at least one n-mer insert is incorporated into the AAV protein such that at least the P motif and/or double valine motif is inserted between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

22. The composition of claim 20, wherein the AAV capsid protein is an engineered AAV capsid protein having reduced or eliminated uptake in a non-CNS cell as compared to a corresponding wild-type AAV capsid polypeptide.

23. The composition of claim 22, wherein the non-CNS cell is a liver cell or a dorsal root ganglion (DRG) neuron.

24. The composition of claim 22, wherein the wild-type capsid polypeptide is an AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

25. The composition of claim 22, wherein the engineered AAV capsid protein comprises one or more mutations that result in reduced or eliminated uptake in a non-CNS cell.

26. The composition of claim 25, wherein the one or more mutations are
a. in position 267,
b. in position 269,
c. in position 272,
d. in position 504,
e. in position 505,
f. in position 585,
g. in position 590,
h. or any combination thereof
in the AAV9 capsid protein (SEQ ID NO: 1) or in one or more positions corresponding thereto in a non-AAV9 capsid polypeptide.

27. The composition of claim 26, wherein the non-AAV9 capsid protein is an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

28. The composition of claim 26, wherein the mutation in position 267 in the AAV9 capsid protein (SEQ ID NO: 1) or position corresponding thereto in a non-AAV9 capsid polypeptide is a G or X mutation to A, wherein X is any amino acid.

29. The composition of claim 26, wherein the mutation in position 269 in the AAV9 capsid protein (SEQ ID NO: 1) or position corresponding thereto in a non-AAV9 capsid polypeptide is an S or X to T mutation, wherein X is any amino acid.

30. The composition of claim 26, wherein the mutation in position 272 in the AAV9 capsid protein (SEQ ID NO: 1) or position corresponding thereto in a non-AAV9 capsid polypeptide is an N or to A mutation, wherein X is any amino acid.

31. The composition of claim 26, wherein the mutation in position 504 in the AAV9 capsid protein (SEQ ID NO: 1) or position corresponding thereto in a non-AAV9 capsid polypeptide is a G or X to A mutation, wherein X is any amino acid.

32. The composition of claim 26, wherein the mutation in position 505 in the AAV9 capsid protein (SEQ ID NO: 1) or position corresponding thereto in a non-AAV9 capsid polypeptide is a P or X to A mutation, wherein X is any amino acid.

33. The composition of claim 26, wherein the mutation in position 585 in the AAV9 capsid protein (SEQ ID NO: 1) or position corresponding thereto in a non-AAV9 capsid polypeptide is an R or X mutation to Q, wherein X is any amino acid.

34. The composition of claim 26, wherein the mutation in position 590 in the AAV9 capsid protein (SEQ ID NO: 1) or position corresponding thereto in a non-AAV9 capsid polypeptide is a Q or X to A mutation, wherein X is any amino acid.

35. The composition of claim 26, wherein the engineered AAV capsid protein is an engineered AAV9 capsid polypeptide comprising a mutation at position 267, position 269 or both of a wild-type AAV9 capsid protein (SEQ ID NO: 1), wherein the mutation at position 267 is a G to A mutation and wherein the mutation at position 269 is an S to T mutation.

36. The composition of claim 26, wherein the engineered AAV capsid protein is an engineered AAV9 capsid polypeptide comprising a mutation at position 590 of a wild-type AAV9 capsid protein (SEQ ID NO: 1), wherein the mutation at position 509 is a Q to A mutation.

37. The composition of claim 26, wherein the engineered AAV capsid protein is an engineered AAV9 capsid polypeptide comprising a mutation at position 504, position 505, or both of a wild-type AAV9 capsid protein (SEQ ID NO: 1), wherein the mutation at position 504 is a G to A mutation and wherein the mutation at position 505 is a P to A mutation.

38. The composition of claim 1, wherein the composition is an engineered viral particle.

39. The composition of claim 38, wherein the engineered viral particle is an engineered AAV viral particle.

40. The composition of claim 39, wherein the AAV viral particle is an engineered AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 viral particle.

41. The composition of claim 1, wherein the optional cargo is capable of treating or preventing a CNS, an eye, or inner ear disease or disorder.

42. The composition of claim 1, wherein the optional cargo comprises one or more specific RNAi molecule binding sequences specific for an RNAi molecule endogenous to a non-target cell, wherein expression of the RNAi molecule(s) is/are enriched in the non-target cell as compared to a CNS cell and/or specific for synthetic RNAi molecule(s).

43. The composition of claim 42, wherein the RNAi molecule is not expressed in a CNS cell.

44. The composition of claim 42, wherein the non-target cell is a liver cell or a dorsal root ganglion neuron.

45. The composition of claim 42, wherein the RNAi molecule is miR183, miR-182, miR122, miR122a, miR99a, miR-26a, miR199a, miRNA-143, miR101a, miR-30c, or any combination thereof.

46. The composition of claim 1, optionally wherein the viral protein is a capsid protein, wherein the composition is modified to
a. include one or more azides,
b. have a reduced number of one or more oxidation susceptible residues, wherein the oxidation susceptible residues are optionally Met, Tyr, Trp, His, Cys or any combination thereof;
c. is PEGylated, or is otherwise functionalized for PEGylation;
d. comprises one or more oligonucleotides tethered via click chemistry to the composition, optionally viral protein;
e. or any combination thereof.

47. A vector system comprising:
a vector comprising:
one or more polynucleotides, wherein at least one of the one or more polynucleotides encodes all or part of a targeting moiety effective to target a central nervous system (CNS) cell, wherein the targeting moiety comprises an n-mer insert optionally comprising or consisting of a P-motif or a double valine motif, or both,
wherein the P-motif comprises or consists of the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7,
wherein the double valine motif comprises or consists of the amino acid sequence XₘX₁X₂VX₃X₄VX₅Xₙ, wherein X₁, X₂, X₃, Xₘ, and Xₙ, are each independently selected from any amino acid, wherein m is 0, 1, 2, or 3, and wherein n is 0, 1, 2, 3, 4, 5, 6, or 7; and
optionally, a regulatory element operatively coupled to one or more of the one or more polynucleotides.

48. The vector system of claim 47, wherein X₂ of the P motif is Q, P, E, or H.

49. The vector system of claim 47, wherein X₁ of the P motif is a polar amino acid, optionally a polar uncharged amino acid.

50. The vector system of claim 47, wherein X₃ of the P motif is a nonpolar amino acid.

51. The vector system of claim 47, wherein X₁ of the double valine motif is R, K, V, or W.

52. The vector system of claim 47, wherein X₂ of the double valine motif is T, S, V, Y or R.

53. The vector system of claim 47, wherein X₃ of the double valine motif is G, P, or S.

54. The vector system of any one of the preceding claims, any one of the preceding claims, wherein X₄ of the double valine motif is S, D, or T.

55. The vector system of claim 47, wherein X₅ of the double valine motif is Y, G, S, or L.

56. The vector system of claim 47, wherein the targeting moiety comprises two or more n-mer inserts, optionally wherein each n-mer insert comprises or consists of a P-motif, wherein at least one of the P-motifs comprise or consists of the amino acid sequence XₘPX₁X₂GTX₃RXₙ (SEQ ID NO: 8579), wherein X₁, X₂, X₃, Xₘ, and Xₙ, are each independently selected from any amino acid, wherein m is 0, 1, 2, or 3, and wherein n is 0, 1, 2, 3, 4, 5, 6, or 7, optionally wherein X₂ of the P motif is Q, P, E, or H, optionally wherein the X₁ of the P motif is a polar amino acid, optionally a polar uncharged amino acid, and optionally wherein X₃ of the P motif is a nonpolar amino acid..

57. The vector system of claim 47, wherein the n-mer insert(s) and/or at least one P-motif and/or double valine motif is selected from any one n-mer insert and/or is encoded by a polynucleotide as set forth in one or more of SEQ ID NOs: 332-582 (Table 7), SEQ ID NOs:
583-8578 (Table 8), SEQ ID NOs: 3-819, 21-22, 24, 200, 202, 204, 212, 218, 224, 226, 228, 286, 234, 258, 260, 647, 649, 923, 1069, 1077, 1265, 2439, 2529, 2759, 3283, 3553, 3923, 4005, 4173, 4537, 4593, 4599, 4601, 4605, 4619, 4665, 4751, 4759, 4825, 4909, 4933, 5013, 5091, 5107, 5127, 5131, 5165, 5177, 5181, 5187, 5189, 5191, 5277, 5287, 5401, 5433, 5631, 5633, 5731, 5741, 5937, 6019, 6045, 6139, 6169, 6497, 7335, 8033, 8269, 8596-8613, (FIGS. 15A, 15B, 17A, 16A, 16B, 16C, and 19A-19C).

58. The vector system of claim 47, wherein the n-mer insert(s) are each 3-25 or 3-15 amino acids in length.

59. The vector system of claim 47, wherein
a. X₁ of the P motif is S, T, N, Q, C, Y or A,
b. X₂ of the P motif is Q, P, E, or H,
c. X₃ is G, A, M, W, L, V, F, or I, or any combination thereof.

60. The vector system of claim 47, further comprising a cargo.

61. The vector system of claim 60, wherein the cargo is a cargo polynucleotide and is optionally operatively coupled to one or more of the one or more polynucleotides encoding the targeting moiety.

62. The vector system of any one of claims 47-61, wherein the vector system is a viral vector system and is capable of producing virus particles, virus particles that contain the cargo, or both.

63. The vector system of claim 47, wherein the vector system is capable of producing a polypeptide comprising one or more of the targeting moieties.

64. The vector system of claim 63, wherein the polypeptide is a viral polypeptide.

65. The vector system of claim 64, wherein the viral polypeptide is a capsid polypeptide.

66. The vector system of claim 65, wherein the capsid polypeptide is an adeno associated virus (AAV) capsid polypeptide.

67. The vector system of claim 62, wherein the virus particles are AAV virus particles.

68. The vector system of claim 67, wherein the AAV virus particles or AAV capsid polypeptide are engineered AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 viral particles or polypeptides.

69. The vector system of claim 64, wherein the n-mer insert(s) is/are incorporated into the viral polypeptide such that at least the n-mer insert(s) is/are located between two amino acids of the viral polypeptide such that at least the n-mer insert(s) is/are external to a viral capsid.

70. The vector system of claim 69, wherein one or more n-mer insert(s) are each incorporated into an AAV capsid polypeptide such that the n-mer insert(s), optionally the P-motif(s) and/or double valine motif(s), are each inserted between any two contiguous amino acids independently selected from amino acids 262-269, 327-332, 382-386, 452-460, 488-505, 527-539, 545-558, 581-593, 598-599, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

71. The vector system of claim 69, wherein the at least one polynucleotide that encodes all or part of a targeting moiety is inserted between the codons corresponding to amino acid 588 and 589 in the AAV9 capsid polynucleotide or in an analogous position in an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

72. The vector system of claim 66, wherein the AAV capsid polypeptide is an engineered AAV capsid polypeptide having reduced or eliminated uptake in a non-CNS cell as compared to a corresponding wild-type AAV capsid polypeptide.

73. The vector system of claim 72, wherein the non-CNS cell is a liver cell or a dorsal root ganglion (DRG) neuron.

74. The vector system of claim 72, wherein the wild-type capsid polypeptide is an AAV1, AAV2, AAV3, AAV4, AAV5, AAV6, AAV7, AAV8, AAV9, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

75. The vector system of claim 72, wherein the engineered AAV capsid polypeptide comprises one or more mutations that result in reduced or eliminated uptake in a non-CNS cell.

76. The vector system of claim 75, wherein the one or more mutations are
a. in position 267,
b. in position 269,
c. in position 272,
d. in position 504,
e. in position 505,
f. in position 585,
g. in position 590,
h. or any combination thereof
in the AAV9 capsid polypeptide (SEQ ID NO: 1) or in one or more positions corresponding thereto in a non-AAV9 capsid polypeptide.

77. The vector system of claim 76, wherein the non-AAV9 capsid polypeptide is an AAV1, AAV2, AAV3, AAV4, AAV5, AAV6, AAV7, AAV8, AAV rh.74, AAV rh.10, AAV12, AAV.DJ, AAV.ie, AAV1.9-3, AAV.Anc80, AAV.Anc80L65, AAV2.7m8, or AAV8BP2 capsid polypeptide.

78. The vector system of claim 76, wherein the mutation in position 267 in the AAV9 capsid polypeptide (SEQ ID NO: 1) or position corresponding thereto in a non-AAV9 capsid polypeptide is a G or X mutation to A, wherein X is any amino acid.

79. The vector system of claim 76, wherein the mutation in position 269 in the AAV9 capsid polypeptide (SEQ ID NO: 1) or position corresponding thereto in a non-AAV9 capsid polypeptide is an S or X to T mutation, wherein X is any amino acid.

80. The vector system of claim 76, wherein the mutation in position 272 in the AAV9 capsid polypeptide (SEQ ID NO: 1) or position corresponding thereto in a non-AAV9 capsid polypeptide is an N or to A mutation, wherein X is any amino acid.

81. The vector system of claim 76, wherein the mutation in position 504 in the AAV9 capsid polypeptide (SEQ ID NO: 1) or position corresponding thereto in a non-AAV9 capsid polypeptide is a G or X to A mutation, wherein X is any amino acid.

82. The vector system of claim 76, wherein the mutation in position 505 in the AAV9 capsid polypeptide (SEQ ID NO: 1) or position corresponding thereto in a non-AAV9 capsid polypeptide is a P or X to A mutation, wherein X is any amino acid.

83. The vector system of claim 76, wherein the mutation in position 585 in the AAV9 capsid polypeptide (SEQ ID NO: 1) or position corresponding thereto in a non-AAV9 capsid polypeptide is an R or X to Q mutation, wherein X is any amino acid.

84. The vector system of claim 76, wherein the mutation in position 590 in the AAV9 capsid polypeptide (SEQ ID NO: 1) or position corresponding thereto in a non-AAV9 capsid polypeptide is a Q or X to A mutation, wherein X is any amino acid.

85. The vector system of claim 76, wherein the engineered AAV capsid polypeptide is an engineered AAV9 capsid polypeptide comprising a mutation at position 267, position 269, or both of a wild-type AAV9 capsid polypeptide (SEQ ID NO: 1), wherein the mutation at position 267 is a G to A mutation and wherein the mutation at position 269 is an S to T mutation.

86. The vector system of claim 76, wherein the engineered AAV capsid polypeptide is an engineered AAV9 capsid polypeptide comprising a mutation at position 590 of a wild-type AAV9 capsid polypeptide (SEQ ID NO: 1), wherein the mutation at position 509 is a Q to A mutation.

87. The vector system of claim 76, wherein the engineered AAV capsid polypeptide is an engineered AAV9 capsid polypeptide comprising a mutation at position 504, position 505, or both of a wild-type AAV9 capsid polypeptide (SEQ ID NO: 1), wherein the mutation at position 504 is a G to A mutation and wherein the mutation at position 505 is a P to A mutation.

88. The vector system of claim 60, wherein the cargo comprises one or more specific RNAi molecule binding sequences specific for an RNAi molecule endogenous to a non-target cell, wherein expression of the RNAi molecule(s) is/are enriched in the non-target cell as compared to a CNS cell and/or specific for synthetic RNAi molecule(s).

89. The vector system of claim 88, wherein the RNAi molecule is not expressed in a CNS cell.

90. The vector system of claim 88, wherein the non-target cell is a liver cell or a dorsal root ganglion neuron.

91. The vector system of claim 88, wherein the RNAi molecule is miR183, miR-182, miR122, miR122a, miR99a, miR-26a, miR199a, miRNA-143, miR101a, miR-30c, or any combination thereof.

92. The vector system of claim 63, optionally wherein the viral polypeptide is a capsid polypeptide, wherein the viral polypeptide is modified to
a. include one or more azides,
b. have a reduced number of one or more oxidation susceptible residues, wherein the oxidation susceptible residues are optionally Met, Tyr, Trp, His, Cys or any combination thereof;
c. is PEGylated, or is otherwise functionalized for PEGylation;
d. comprises one or more oligonucleotides tethered via click chemistry to the composition, optionally viral protein;
e. or any combination thereof.

93. The vector system of claim 63, wherein the viral vector and/or cargo is engineered to include one or more cis-acting elements or modifications, optionally
a. a reduced number of CpG islands;
b. one or more TLR9i oligonucleotides, optionally in one or both of the inverted terminal repeats of the vector system;
c. one or more regulatory elements to modify cargo expression;
d. a reduced number of ITR mimicking harpin or other structures;
e. or any combination thereof.

94. The vector system of claim 47, wherein the vector comprising the one or more polynucleotides does not comprise splice regulatory elements.

95. The vector system of claim 47, further comprising a polynucleotide that encodes a viral rep protein.

96. The vector system of claim 95, wherein the viral rep protein is an AAV rep protein.

97. The vector system of claim 95, wherein the polynucleotide that encodes the viral rep protein is on the same vector or a different vector as the one or more polynucleotides.

98. The vector system of claim 95, wherein the polynucleotide that encodes the viral rep protein is operatively coupled to a regulatory element.

99. The vector system of claim 47, wherein the vector system encodes and/or is capable of producing a composition or portion thereof as in any one of claims 1-46.

100. A polynucleotide encoding a composition or portion thereof as in any one of claims 1-46.

101. A polypeptide encoded by and/or produced by a vector system as in any of claims 47-99, or a polynucleotide of claim 100.

102. The polypeptide of claim 101, wherein the polypeptide is a viral polypeptide.

103. The polypeptide of claim 102, wherein the viral polypeptide is an AAV polypeptide.

104. The polypeptide of claim 101, wherein the polypeptide is coupled to or otherwise associated with a cargo.

105. The polypeptide of claim 104, wherein the cargo comprises one or more specific RNAi molecule binding sequences specific for an RNAi molecule endogenous to a non-target cell, wherein expression of the RNAi molecule(s) is/are enriched in the non-target cell as compared to a CNS cell and/or specific for synthetic RNAi molecule(s).

106. The polypeptide of claim 105, wherein the RNAi molecule is not expressed in a CNS cell.

107. The polypeptide of claim 104, wherein the non-target cell is a liver cell or a dorsal root ganglion neuron.

108. The polypeptide of claim 104, wherein the RNAi molecule is miR183, miR-182, miR122, miR122a, miR99a, miR-26a, miR199a, miRNA-143, miR101a, miR-30c, or any combination thereof.

109. The polypeptide of claim 101, wherein the polypeptide includes one or more azides; has a reduced number of one or more oxidation susceptible residues, wherein the oxidation susceptible residues are optionally Met, Tyr, Trp, His, Cys or any combination thereof; is PEGylated, or is otherwise functionalized for PEGylation; comprises one or more oligonucleotides tethered via click chemistry to the composition, optionally viral protein; or any combination thereof.

110. A particle produced by a vector system as in any one of claims 47-99, optionally including a polypeptide as in any one of claims 101-109.

111. The particle of claim 110, wherein the particle is a viral particle.

112. The particle of claim 111, wherein the viral particle is an adeno-associated virus (AAV) particle, lentiviral particle, or a retroviral particle.

113. The particle of claim 110, wherein the particle comprises a cargo.

114. The particle of claim 110, wherein the viral particle has a central nervous system (CNS) tropism.

115. The particle of claim 110, wherein the cargo comprises one or more specific RNAi molecule binding sequences specific for an RNAi molecule endogenous to a non-target cell, wherein expression of the RNAi molecule(s) is/are enriched in the non-target cell as compared to a CNS cell and/or specific for synthetic RNAi molecule(s).

116. The particle of claim 115, wherein the RNAi molecule is not expressed in a CNS cell.

117. The particle of claim 115, wherein the non-target cell is a liver cell or a dorsal root ganglion neuron.

118. The particle of claim 115, wherein the RNAi molecule is miR183, miR-182, miR122, miR122a, miR99a, miR-26a, miR199a, miRNA-143, miR101a, miR-30c, or any combination thereof.

119. The particle of claim 110, wherein the polypeptide includes one or more azides; has a reduced number of one or more oxidation susceptible residues, wherein the oxidation susceptible residues are optionally Met, Tyr, Trp, His, Cys or any combination thereof; is PEGylated, or is otherwise functionalized for PEGylation; comprises one or more oligonucleotides tethered via click chemistry to the composition, optionally viral protein; or any combination thereof.

120. The vector system of any one of claims 47-99, the polypeptide as in any one of claims 100-109, or the particle of any one of claims 110-119, wherein the cargo is capable of treating or preventing a CNS, an eye, or an inner ear disease or disorder.

121. A cell comprising:
a. a composition as in any of claims 1-46;
b. a vector system as in any one of claims 66-99 or 120;
c. a polynucleotide as in claim 100;
d. a polypeptide as in any one of claims 101-109 or 120;
e. a particle of any one of claims 110-120; or
f. any combination thereof.

122. The cell of claim 121, wherein the cell is prokaryotic.

123. The cell of claim 121, wherein the cell is eukaryotic.

124. A pharmaceutical formulation comprising:
a. a composition as in any of claims 1-46;
b. a vector system as in any one of claims 66-99 or 120;
c. a polynucleotide as in claim 100;
d. a polypeptide as in any one of claims 101-109 or 120;
e. a particle of any one of claims 110-120;
f. a cell as in any one of claims 121-123; or
any combination thereof; and
a pharmaceutically acceptable carrier.

125. A method of treating a central nervous system, an eye, an inner ear, a pain disease, disorder, or a symptom thereof or a pain comprising:
administering, to the subject in need thereof,
a. a composition as in any of claims 1-46;
b. a vector system as in any one of claims 66-99 or 120;
c. a polynucleotide as in claim 100;
d. a polypeptide as in any one of claims 101-109 or 120;
e. a particle of any one of claims 110-120;
f. a cell as in any one of claims 121-123;
g. a pharmaceutical formulation as in claim 124; or
h. any combination thereof.

126. The method of claim 125, wherein the central nervous system disease or disorder comprises a secondary muscle disease, disorder, or symptom thereof.

127. The method of any one of claims 125-126, wherein the central nervous system disease or disorder is Friedreich's Ataxia, Dravet Syndrome, Spinocerebellar Ataxia Type 3, Niemann Pick Type C, Huntington's Disease, Pompe Disease, Myotonic Dystrophy Type 1, Glut1 Deficiency Syndrome (De Vivo Syndrome), Tay-Sachs, Spinal Muscular Atrophy, Alzheimer's disease, Amyotrophic lateral sclerosis (ALS), Danon disease, Rett Syndrome, Angleman Syndrome, infantile neuronal dystorpy, Gaucher's disease, Krabbe disease, metachromatic leukodystrophy, Salla disease, Farber disease or Spinal Musular Atrophy with progressive myoclonic Epilepsy (also reffered to as Jankovic-Rivera syndrome, Unverricht-Lundborg disease, AADC deficiency, Parkinson's disease, Batten disease, a neuronal ceroid lipofuscinosis disease, giant axonal neuropathy, a mucopolysaccharidosis disease (e.g., Hurler syndrome, MPS III A-D), neurofibromatosis, a spinocerebellar ataxia disease, Sandoff disease, GM2 gangliosidosis, Canavan disease, Cockayne syndrome, a pain disease or disorder, a pain, a neuropathy or any combination thereof.

128. The method of any one of claims 125-127, wherein the eye disease or disorder is Stargardt disease, a Leber's congenital amaurosis (LCA) (e.g., Leber's congenital amaurosis type 2, LEBER CONGENITALAMAUROSIS (LCA) ANDEARLY-ONSET SEVERE RETINALDYSTROPHY (EOSRD)), Choroideremia, a macular degeneration, diabetic retinopathy, a retinopathy, vitelliform macular dystrophy, a macular dystrophy, Sorsby's fundus dystrophy, cataracts, glaucoma, optic neuropathies, Marfan syndrome, myopia, polypoidal choroidal vasculopathies, retinitis pigmentosa, uveal melanoma, X-linked retinoschisis, pattern dystrophy, achromatopsia, Blue cone monochromatism, Bornholm eye disease, ADGUCA1A-associated COD/CORD, autosomal dominant PRPH2 associated CORD, X-linkedRPGR-associatedCOD/CORD, fundus albipunctatus, Enhanced S-conesyndrome, Bietti crystalline comeoretinaldystorphy, or any combination thereof.

129. The method of any one of claims 125-128, wherein the inner ear disease or disorder is GJB-2 deafness, Jeryell and Lange-Nielsen syndrome, Usher syndrome, Alport syndrome, Branchio-oto-renal syndrome, Waardenburg syndrome, Pendred syndrome, Stickler syndrome, Treacher Collins syndrome, CHARGE syndrome, Norrie disease, Perrault syndrome, Autosomal dominant Nonsyndromic hearing loss, utosomal Recessive Nonsyndromic Hearing Loss, X-linked nonsyndromic hearing loss, an auditory neuropathy, a congenital hearing loss, or any combination thereof.
